(19) United States
(12) Patent Application Publication
(10) Pub. No.: US 2006/0014153 A1
(43) Pub. Date: Jan. 19, 2006

(54) Novel proteins and nucleic acids encoding same

(76) Inventors: Valerie L. Gerlach, Branford, CT (US); John R. MacDougall, Hamden, CT (US); Glennda Smithson, Guilford, CT (US); Isabelle Millet, Milford, CT (US); David Stone, Guilford, CT (US); Erik Gunther, Branford, CT (US); Karen Ellerman, Branford, CT (US); William M. Grosse, Branford, CT (US); John P. Alsobrook II, Madison, CT (US); Denise M. Lepley, Branford, CT (US); Catherine E. Burgess, Wethersfield, CT (US); Muralidhara Padigaru, Branford, CT (US); Ramesha Kekuda, Norwalk, CT (US); Kimberly A. Spytek, New Haven, CT (US); Martin D. Leach, Madison, CT (US); Richard A. Shimkets, Guilford, CT (US)

Correspondence Address: Jenell Lawson;Intellectual Property, CuraGen Corporation, 555 Long Wharf Drive, New Haven, CT, 06511, US

(21) Appl. No.: 10/877,346

(22) Filed: Jun. 25, 2004

Related U.S. Application Data
(63) Continuation of application No. 09/964,956, filed on Sep. 26, 2001, now Pat. No. 6,875,570.
(60) Provisional application No. 60/235,631, filed on Sep. 27, 2000.
(60) Provisional application No. 60/235,633, filed on Sep. 27, 2000.
(60) Provisional application No. 60/235,808, filed on Sep. 27, 2000.
(60) Provisional application No. 60/236,064, filed on Sep. 27, 2000.
(60) Provisional application No. 60/236,065, filed on Sep. 27, 2000.
(60) Provisional application No. 60/236,066, filed on Sep. 27, 2000.
(60) Provisional application No. 60/236,165, filed on Sep. 29, 2000.
(60) Provisional application No. 60/237,434, filed on Oct. 3, 2000.
(60) Provisional application No. 60/238,321, filed on Oct. 5, 2000.
(60) Provisional application No. 60/238,399, filed on Oct. 6, 2000.
(60) Provisional application No. 60/238,896, filed on Oct. 10, 2000.
(60) Provisional application No. 60/276,667, filed on Mar. 16, 2001.
(60) Provisional application No. 60/294,823, filed on May 31, 2001.
(60) Provisional application No. 60/304,868, filed on Jul. 12, 2001.

(51) Int. Cl. C12Q 1/68; C07H 21/04; C12P 21/06; C12N 9/00

(52) U.S. Cl. 435/6; 435/69.1; 435/183; 435/320.1; 435/325; 530/350; 536/23.2

(57) ABSTRACT

Disclosed herein are nucleic acid sequences that encode novel polypeptides. Also disclosed are polypeptides encoded by these nucleic acid sequences, and antibodies, which immunospecifically-bind to the polypeptide, as well as derivatives, variants, mutants, or fragments of the aforementioned polypeptide, polynucleotide, or antibody. The invention further discloses therapeutic, diagnostic and research methods for diagnosis, treatment, and prevention of disorders involving any one of these novel human nucleic acids and proteins.

### RELATED APPLICATIONS

This application claims priority from U.S. Ser. No. 60/235,631, filed Sep. 27, 2000; U.S. Ser. No. 60/235,633, filed Sep. 27, 2000; U.S. Ser. No. 60/235,808, filed Sep. 27, 2000; U.S. Ser. No. 60/236,064 filed Sep. 27, 2000; U.S. Ser. No. 60/236,065, filed Sep. 27, 2000; U.S. Ser. No. 60/236,066, filed Sep. 27, 2000; U.S. Ser. No. 60/236,165, filed Sep. 28, 2000; U.S. Ser. No. 60/237,434, filed Oct. 3, 2000; U.S. Ser. No. 60/238,321, filed Oct. 5, 2000; U.S. Ser. No. 60/238,399, filed Oct. 6, 2000; U.S. Ser. No. 60/238,896, filed Oct. 6, 2000; U.S. Ser. No. 60/276,667, filed Mar. 16, 2001; U.S. Ser. No. 60/294,823, filed May 31,2001; and U.S. Ser. No. 60/304,868, filed Jul. 12, 2001 each of which is incorporated by reference in its entirety.

### FIELD OF THE INVENTION

The invention generally relates to nucleic acids and polypeptides encoded thereby.

### BACKGROUND OF THE INVENTION

The invention generally relates to nucleic acids and polypeptides encoded therefrom. More specifically, the invention relates to nucleic acids encoding cytoplasmic, nuclear, membrane bound, and secreted polypeptides, as well as vectors, host cells, antibodies, and recombinant methods for producing these nucleic acids and polypeptides.

### SUMMARY OF THE INVENTION

The invention is based in part upon the discovery of nucleic acid sequences encoding novel polypeptides. The novel nucleic acids and polypeptides are referred to herein as NOVX, or NOV1, NOV2, NOV3, NOV4, NOV5, NOV6, NOV7, and NOV8 nucleic acids and polypeptides. These nucleic acids and polypeptides, as well as derivatives, homologs, analogs and fragments thereof, will hereinafter be collectively designated as “NOVX” nucleic acid or polypeptide sequences.

In one aspect, the invention provides an isolated NOVX nucleic acid molecule encoding a NOVX polypeptide that includes a nucleic acid sequence that has identity to the nucleic acids disclosed in SEQ ID NOS:1, 3, 4, 6, 8, 10, 12, 14, 16, 18, 20, 22, 24, and 26. In some embodiments, the NOVX nucleic acid molecule will hybridize under stringent conditions to a nucleic acid sequence complementary to a nucleic acid molecule that includes a protein-coding sequence of a NOVX nucleic acid sequence. The invention also includes an isolated nucleic acid that encodes a NOVX polypeptide, or a fragment, homolog, analog or derivative thereof. For example, the nucleic acid can encode a polypeptide at least 80% identical to a polypeptide comprising the amino acid sequences of SEQ ID NOS:2, 5, 7, 9, 11, 13, 15, 17, 19, 21, 23, 25, and 27. The nucleic acid can be, for example, a genomic DNA fragment or a cDNA molecule that includes the nucleic acid sequence of any of SEQ ID NOS:1, 3,4, 6, 8, 10, 12, 14, 16, 18, 20, 22,24, and 26.

Also included in the invention is an oligonucleotide, e.g., an oligonucleotide which includes at least 6 contiguous nucleotides of a NOVX nucleic acid (e.g., SEQ ID NOS:1, 3, 4, 6, 8, 10, 12, 14, 16, 18, 20, 22, 24, and 26) or a complement of said oligonucleotide.

Also included in the invention are substantially purified NOVX polypeptides (SEQ ID NOS:2, 5, 7, 9, 11, 13, 15, 17, 19, 21, 23, 25, and 27). In certain embodiments, the NOVX polypeptides include an amino acid sequence that is substantially identical to the amino acid sequence of a human NOVX polypeptide.

The invention also features antibodies that immunoselectively bind to NOVX polypeptides, or fragments, homologs, analogs or derivatives thereof.

In another aspect, the invention includes pharmaceutical compositions that include therapeutically- or prophylactically-effective amounts of a therapeutic and a pharmaceutically-acceptable carrier. The therapeutic can be, e.g., a NOVX nucleic acid, a NOVX polypeptide, or an antibody specific for a NOVX polypeptide. In a further aspect, the invention includes, in one or more containers, a therapeutically- or prophylactically-effective amount of this pharmaceutical composition.

In a further aspect, the invention includes a method of producing a polypeptide by culturing a cell that includes a NOVX nucleic acid, under conditions allowing for expression of the NOVX polypeptide encoded by the DNA. If desired, the NOVX polypeptide can then be recovered.

In another aspect, the invention includes a method of detecting the presence of a NOVX polypeptide in a sample. In the method, a sample is contacted with a compound that selectively binds to the polypeptide under conditions allowing for formation of a complex between the polypeptide and the compound. The complex is detected, if present, thereby identifying the NOVX polypeptide within the sample.

The invention also includes methods to identify specific cell or tissue types based on their expression of a NOVX.

Also included in the invention is a method of detecting the presence of a NOVX nucleic acid molecule in a sample by contacting the sample with a NOVX nucleic acid probe or primer, and detecting whether the nucleic acid probe or primer bound to a NOVX nucleic acid molecule in the sample.

In a further aspect, the invention provides a method for modulating the activity of a NOVX polypeptide by contacting a cell sample that includes the NOVX polypeptide with a compound that binds to the NOVX polypeptide in an amount sufficient to modulate the activity of said polypeptide. The compound can be, e.g., a small molecule, such as a nucleic acid, peptide, polypeptide, peptidomimetic, carbohydrate, lipid or other organic (carbon containing) or inorganic molecule, as further described herein.

Also within the scope of the invention is the use of a therapeutic in the manufacture of a medicament for treating or preventing disorders or syndromes including, e.g., Cancer, Leukodystrophies, Breast cancer, Ovarian cancer, Prostate cancer, Uterine cancer, Hodgkin disease, Adenocarcinoma, Adrenoleukodystrophy, Cystitis, incontinence, Von Hippel-Lindau (VHL) syndrome, hypercalceimia, Endometriosis, Hirschsprung's disease, Crohn's Disease, Appendicitis, Cirrhosis, Liver failure, Wolfram Syndrome, Smith-Lemli-Opitz syndrome, Retinitis pigmentosa, Leigh syndrome; Congenital Adrenal Hyperplasia, Xerostomia; tooth decay and other dental problems; Inflammatory bowel disease, Diverticular disease, fertility, Infertility, cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, Hemophilia, Hypercoagulation, Idiopathic thrombocytopenic purpura, obesity, Diabetes Insipidus and Mellitus with Optic Atrophy and Deafness, Pancreatitis, Metabolic Dysregulation, transplantation recovery, Autoimmune disease, Systemic lupus erythematosus, asthma, arthritis, psoriasis, Emphysema, Scleroderma, allergy, ARDS, Immunodeficiencies, Graft vesus host, Alzheimer's disease, Stroke, Parkinson's disease, Huntington's disease, Cerebral palsy, Epilepsy, Multiple sclerosis, Ataxia-telangiectasia, Behavioral disorders, Addiction, Anxiety, Pain, Neurodegeneration, Muscular dystrophy, Lesch-Nyhan syndrome, Myasthenia gravis, schizophrenia, and other dopamine-dysfunctional states, levodopa-induced dyskinesias, alcoholism, pileptic seizures and other neurological disorders, mental depression, Cerebellar ataxia, pure; Episodic ataxia, type 2; Hemiplegic migraine, Spinocerebellar ataxia-6, Tuberous sclerosis, Renal artery stenosis, Interstitial nephritis, Glomerulonephritis, Polycystic kidney disease, Renal tubular acidosis, IgA nephropathy, and/or other pathologies and disorders of the like.

The therapeutic can be, e.g., a NOVX nucleic acid, a NOVX polypeptide, or a NOVX-specific antibody, or biologically-active derivatives or fragments thereof.

For example, the compositions of the present invention will have efficacy for treatment of patients suffering from the diseases and disorders disclosed above and/or other pathologies and disorders of the like. The polypeptides can be used as immunogens to produce antibodies specific for the invention, and as vaccines. They can also be used to screen for potential agonist and antagonist compounds. For example, a cDNA encoding NOVX may be useful in gene therapy, and NOVX may be useful when administered to a subject in need thereof. By way of non-limiting example, the compositions of the present invention will have efficacy for treatment of patients suffering from the diseases and disorders disclosed above and/or other pathologies and disorders of the like.

The invention further includes a method for screening for a modulator of disorders or syndromes including, e.g., the diseases and disorders disclosed above and/or other pathologies and disorders of the like. The method includes contacting a test compound with a NOVX polypeptide and determining if the test compound binds to said NOVX polypeptide. Binding of the test compound to the NOVX polypeptide indicates the test compound is a modulator of activity, or of latency or predisposition to the aforementioned disorders or syndromes.

Also within the scope of the invention is a method for screening for a modulator of activity, or of latency or predisposition to disorders or syndromes including, e.g., the diseases and disorders disclosed above and/or other pathologies and disorders of the like by administering a test compound to a test animal at increased risk for the aforementioned disorders or syndromes. The test animal expresses a recombinant polypeptide encoded by a NOVX nucleic acid. Expression or activity of NOVX polypeptide is then measured in the test animal, as is expression or activity of the protein in a control animal which recombinantly-expresses NOVX polypeptide and is not at increased risk for the disorder or syndrome. Next, the expression of NOVX polypeptide in both the test animal and the control animal is compared. A change in the activity of NOVX polypeptide in the test animal relative to the control animal indicates the test compound is a modulator of latency of the disorder or syndrome.

In yet another aspect, the invention includes a method for determining the presence of or predisposition to a disease associated with altered levels of a NOVX polypeptide, a NOVX nucleic acid, or both, in a subject (e.g., a human subject). The method includes measuring the amount of the NOVX polypeptide in a test sample from the subject and comparing the amount of the polypeptide in the test sample to the amount of the NOVX polypeptide present in a control sample. An alteration in the level of the NOVX polypeptide in the test sample as compared to the control sample indicates the presence of or predisposition to a disease in the subject. Preferably, the predisposition includes, e.g., the diseases and disorders disclosed above and/or other pathologies and disorders of the like. Also, the expression levels of the new polypeptides of the invention can be used in a method to screen for various cancers as well as to determine the stage of cancers.

In a further aspect, the invention includes a method of treating or preventing a pathological condition associated with a disorder in a mammal by administering to the subject a NOVX polypeptide, a NOVX nucleic acid, or a NOVX-specific antibody to a subject (e.g., a human subject), in an amount sufficient to alleviate or prevent the pathological condition. In preferred embodiments, the disorder, includes, e.g., the diseases and disorders disclosed above and/or other pathologies and disorders of the like.

In yet another aspect, the invention can be used in a method to identity the cellular receptors and downstream effectors of the invention by any one of a number of techniques commonly employed in the art. These include but are not limited to the two-hybrid system, affinity purification, co-precipitation with antibodies or other specific-interacting molecules.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

The present invention provides novel nucleotides and polypeptides encoded thereby. Included in the invention are the novel nucleic acid sequences and their encoded polypeptides. The sequences are collectively referred to herein as “NOVX nucleic acids” or “NOVX polynucleotides” and the corresponding encoded polypeptides are referred to as “NOVX polypeptides” or “NOVX proteins.” Unless indicated otherwise, “NOVX” is meant to refer to any of the novel sequences disclosed herein. Table A provides a summary of the NOVX nucleic acids and their encoded polypeptides.

TABLE A
Sequences and Corresponding SEQ ID Numbers

| | | SEQ ID | | |
|---|---|---|---|---|
| | | NO | | |
| NOVX | | (nucleic | SEQ ID NO | |
| Assignment | Internal Identification | acid) | (polypeptide) | Homology |
| 1a | 83420733_EXT | 1 | 2 | Insulin Like Growth Factor |
| | | | | Binding Protein Complex- |
| | | | | Acid Labile Subunit-like |
| 1b | AL356413.6 | 3 | 2 | Insulin Like Growth Factor |
| | | | | Binding Protein Complex- |
| | | | | Acid Labile Subunit-like |
| 1c | CG52997-02 | 4 | 5 | Insulin Like Growth Factor |
| | | | | Binding Protein Complex- |
| | | | | Acid Labile Subunit-like |
| 2 | 101599929_EXT1 | 6 | 7 | Attractin like |
| 3a | 124217931_EXT | 8 | 9 | Kinase-like |
| 3b | 124217931 | 10 | 11 | RHO/RAC-interacting citron |
| | | | | kinase-like |
| 4 | 105827550_EXT | 12 | 13 | Plexin-like |
| 5 | GMAC027612_A | 14 | 15 | Dopamine receptor-like |
| 6 | GM523_e_1_A | 16 | 17 | metabotropic Glutamate |
| | | | | Receptor |
| 7a | sggc_draft_ba560a15_{—} | 18 | 19 | PV-1-like |
| | 20000723_da1 | | | |
| 7b | 2847264.0.32 | 20 | 21 | PV-1-like |
| 7c | CG51878-03 | 22 | 23 | PV-1-like |
| 8a | SC134914330_A | 24 | 25 | Papin-like |
| 8b | CG57026-04 | 26 | 27 | Papin-like |

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

NOV1 is homologous to a Insulin Like Growth Factor Binding Protein Complex-Acid Labile Subunit-like family of proteins. Thus, the NOVI nucleic acids, polypeptides, antibodies and related compounds according to the invention will be useful in therapeutic and diagnostic applications implicated in, for example; cancer, cystitis, incontinence, fertility, cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation recovery, and/or other pathologies/disorders.

NOV2 is homologous to the Attractin-like family of proteins. Thus NOV2 nucleic acids, polypeptides, antibodies and related compounds according to the invention will be useful in therapeutic and diagnostic applications implicated in, for example; Von Hippel-Lindau (VHL) syndrome, Alzheimer's disease, Stroke, Tuberous sclerosis, hypercalceimia, Parkinson's disease, Huntington's disease, Cerebral palsy, Epilepsy, Multiple sclerosis, Ataxia-telangiectasia, Leukodystrophies, Behavioral disorders, Addiction, Anxiety, Pain, Neurodegeneration, Diabetes, Autoimmune disease, Renal artery stenosis, Interstitial nephritis, Glomerulonephritis, Polycystic kidney disease, Systemic lupus erythematosus, Renal tubular acidosis, IgA nephropathy, Hypercalceimia, Diabetes, Pancreatitis, Obesity, Endometriosis, Infertility, Hirschsprung's disease, Crohn's Disease, Appendicitis, Muscular dystrophy, Lesch-Nyhan syndrome, Myasthenia gravis, Cirrhosis, Liver failure, Breast cancer, Ovarian cancer, Prostate cancer, Uterine cancer and/or other pathologies/disorders.

NOV3 is homologous to a family of RHO/RAC-interacting citron kinase-like proteins. Thus, the NOV3 nucleic acids and polypeptides, antibodies and related compounds according to the invention will be useful in therapeutic and diagnostic applications implicated in, for example: asthma, arthritis, psoriasis, diabetes, and IBD, which require activated T cells, as well as diseases such as systemic lupus erythematosus that involve B cell activation, Autoimmune disease, Renal artery stenosis, Interstitial nephritis, Glomerulonephritis, Polycystic kidney disease, Renal tubular acidosis, IgA nephropathy, Hypercalceimia, Lesch-Nyhan syndrome, Von Hippel-Lindau (VHL) syndrome, Alzheimer's disease, Stroke, Tuberous sclerosis, hypercalceimia, Parkinson's disease, Huntington's disease, Cerebral palsy, Epilepsy, Multiple sclerosis, Ataxia-telangiectasia, Leukodystrophies, Behavioral disorders, Addiction, Anxiety, Pain, Neuroprotection, Endocrine dysfunctions, Obesity, Growth and Reproductive disorders Hemophilia, hypercoagulation, idiopathic thrombocytopenic purpura, allergies, immunodeficiencies, transplantation, Lymphaedema, Hemophilia, Hypercoagulation,Idiopathic thrombocytopenic purpura, Immunodeficiencies, Graft vesus host, Hirschsprung's disease, Crohn's Disease, Appendicitis Inflammatory bowel disease, Diverticular disease and/or other pathologies/disorders.

NOV4 is homologous to the Plexin-like family of proteins. Thus, NOV4 nucleic acids, polypeptides, antibodies and related compounds according to the invention will be useful in therapeutic and diagnostic applications implicated in, for example: Von Hippel-Lindau (VHL) syndrome, Alzheimer's disease, Stroke, Tuberous sclerosis, hypercalceimia, Parkinson's disease, Huntington's disease, Cerebral palsy, Epilepsy, Lesch-Nyhan syndrome, Multiple sclerosis, Ataxia-telangiectasia, Leukodystrophies, Behavioral disorders, Addiction, Anxiety, Pain, Neurodegeneration, Systemic lupus erythematosus, Autoimmune disease, Asthma, Emphysema, Scleroderma, allergy, ARDS, Obesity, Metabolic Dysregulation, Infertility and/or other pathologies/disorders.

NOV5 is homologous to the Dopamine receptor-like family of proteins. Thus NOV5 nucleic acids, polypeptides, antibodies and related compounds according to the invention will be useful in therapeutic and diagnostic applications implicated in, for example: schizophrenia, and other dopamine-dysfunctional states, Hypertension, Huntington's disease, levodopa-induced dyskinesias, alcoholism, Diabetes Insipidus and Mellitus with Optic Atrophy and Deafness, Wolfram Syndrome and/or other pathologies/disorders.

NOV6 is homologous to the Metabotropic Glutamate Receptor-like family of proteins. Thus NOV6 nucleic acids, polypeptides, antibodies and related compounds according to the invention will be useful in therapeutic and diagnostic applications implicated in, for example: pileptic seizures and other neurological disorders, Hodgkin disease, polycystic kidney disease, mental depression, Adenocarcinoma, Smith-Lemli-Opitz syndrome, Retinitis pigmentosa and/or other pathologies/disorders.

NOV7 is homologous to members of the PV-1-like family of proteins. Thus, the NOV7 nucleic acids, polypeptides, antibodies and related compounds according to the invention will be useful in therapeutic and diagnostic applications implicated in, for example; Cerebellar ataxia, pure; Episodic ataxia, type 2; Hemiplegic migraine, familial; Leigh syndrome; Spinocerebellar ataxia-6; Psoriasis, susceptibility to; Autoimmune disease, Asthma, Emphysema, Scleroderma, allergy, ARDS, Von Hippel-Lindau (VHL) syndrome, Alzheimer's disease, Stroke, Tuberous sclerosis, hypercalceimia, Parkinson's disease, Huntington's disease, Cerebral palsy, Epilepsy, Lesch-Nyhan syndrome, Multiple sclerosis, Ataxia-telangiectasia, Leukodystrophies, Behavioral disorders, Addiction, Anxiety, Pain, Neuroprotection, Muscular dystrophy, Myasthenia gravis, Hemophilia, Hypercoagulation, Idiopathic thrombocytopenic purpura, Immunodeficiencies, Graft vesus host, Von Hippel-Lindau (VHL) syndrome, Cirrhosis, Transplantation, Cardiomyopathy, Atherosclerosis, Hypertension, Congenital heart defects, Aortic stenosis, Atrial septal defect (ASD), Atrioventricular (A-V) canal defect, Ductus arteriosus, Pulmonary stenosis, Subaortic stenosis, Ventricular septal defect (VSD), valve diseases, Scleroderma, Obesity, Transplantation; fertility; cancer; Renal artery stenosis, Interstitial nephritis, Glomerulonephritis, Polycystic kidney disease, Systemic lupus erythematosus, Renal tubular acidosis, IgA nephropathy, Hypercalceimia, Lesch-Nyhan syndrome, Adrenoleukodystrophy, Congenital Adrenal Hyperplasia, Xerostomia; tooth decay and other dental problems; Inflammatory bowel disease, Diverticular disease, Pancreatitis, and/or other pathologies/disorders.

NOV8 is homologous to the Papin-like family of proteins. Thus, NOV8 nucleic acids and polypeptides, antibodies and related compounds according to the invention will be useful in therapeutic and diagnostic applications implicated in, for example; cancer, cystitis, incontinence, fertility, cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation recovery and/or other pathologies/disorders.

The NOVX nucleic acids and polypeptides can also be used to screen for molecules, which inhibit or enhance NOVX activity or function. Specifically, the nucleic acids and polypeptides according to the invention may be used as targets for the identification of small molecules that modulate or inhibit, e.g., neurogenesis, cell differentiation, cell proliferation, hematopoiesis, wound healing and angiogenesis.

Additional utilities for the NOVX nucleic acids and polypeptides according to the invention are disclosed herein.

NOV1

NOV1 includes three novel Insulin Like Growth Factor Binding Protein Complex-Acid Labile Subunit (IGFBP-ALS)-like proteins disclosed below. The disclosed sequences have been named NOV1a, NOV1b, and NOV1c. The nucleotide sequences for NOV1a and b both code for the NOV1 a protein sequence. The NOV1c nucleic acid sequence codes for the NOV1c protein sequence.

NOV1a

A disclosed NOV1a nucleic acid of 2838 nucleotides (also referred to as 83420733_EXT) encoding a novel Insulin Like Growth Factor Binding Protein Complex-Acid Labile Subunit-like protein is shown in Table 1A. An open reading frame was identified beginning with an ATG initiation codon at nucleotides 184-186 and ending with a TAG codon at nucleotides 2707-2709. A putative untranslated region upstream from the initiation codon and downstream from the termination codon is underlined in Table 1A. The start and stop codons are in bold letters.

TABLE 1A
NOV1a nucleotide sequence (SEQ ID NO: 1).

AACTTTATGAAGCTATGGGACTTGACAAAAAGTGATATTTGAGAAGAAAGTACGCAGTGGTTGGTGTTTTCT
TTTTTTTAATAAAGGAATTGAATTACTTTGAACACCTCTTCCAGCTGTGCATTACAGATAACGTCAGGAAGA
GTCTCTGCTTTACAGAATCGGATTTCATCACATGACAACATGAAGCTGTGGATTCATCTCTTTTATTCATCT
CTCCTTGCCTGTATATCTTTACACTCCCAAACTCCAGTGCTCTCATCCAGAGGCTCTTGTGATTCTCTTTGC
AATTGTGAGGAAAAAGATGGCACAATGCTAATAAATTGTGAAGCAAAAGGTATCAAGATGGTATCTGAAATA
AGTGTGCCACCATCACGACCTTTCCAACTAAGCTTATTAAATAACGGCTTGACGATGCTTCACACAAATGAC
TTTTCTGGGCTTACCAATGCTATTTCAATACACCTTGGATTTAACAATATTGCAGATATTGAGATAGGTGCA
TTTAATGGCCTTGGCCTCCTGAAACAACTTCATATCAATCACAATTCTTTAGAAATTCTTAAAGAGGATACT
TTCCATGGACTGGAAAACCTGGAATTCCTGCAACCAGATAACAATTTTATCACAGTGATTGAACCAAGTGCC
TTTAGCAAGCTCAACAGACTCAAAGTGTTAATTTTAAATGACAATGCTATTGAGAGTCTTCCTCCAAACATC
TTCCGATTTGTTCCTTTAACCCATCTAGATCTTCGTGGAAATCAATTACAAACATTGCCTTATGTTGGTTTT
CTCGAACACATTGGCCGAATATTCGATCTTCAGTTGGAGGACAACAAATGGGCCTGCAATTGTGACTTATTG
CAGTTAAAAACTTGGTTGGAGAACATGCCTCCACAGTCTATAATTGGTGATGTTGTCTGCAACAGCCCTCCA
TTTTTTAAAGGAAGTATACTCAGTAGACTAAAGAAGGAATCTATTTGCCCTACTCCACCAGTGTATGAAGAA
CATGAGGATCCTTCAGGATCATTACATCTGGCAGCAACATCTTCAATAAATGATAGTCGCATGTCAACTAAG
ACCACGTCCATTCTAAAACTACCCACCAAAGCACCAGGTTTGATACCTTATATTACAAAGCCATCCACTCAA
CTTCCAGGACCTTACTGCCCTATTCCTTGTAACTGCAAAGTCCTATCCCCATCAGGACTTCTAATACATTGT
CAGGAGCGCAACATTGAAAGCTTATCAGATCTGAGACCTCCTCCGCAAAATCCTAGAAAGCTCATTCTAGCG
GGAAATATTATTCACAGTTTAATGAAGTCTGATCTAGTGGAATATTTCACTTTGGAAATGCTTCACTTGGGA
AACAATCGTATTGAAGTTCTTGAAGAAGGATCGTTTATGAACCTAACGAGATTACAAAAACTCTATCTAAAT
GGTAACCACCTGACCAAATTAAGTAAAGGCATGTTCCTTGGTCTCCATAATCTTGAATACTTATATCTTGAA
TACAATGCCATTAAGGAAATACTGCCAGGAACCTTTAATCCAATGCCTAAACTTAAAGTCCTGTATTTAAAT
AACAACCTCCTCCAAGTTTTACCACCACATATTTITTCAGGGGTTCCTCTAACTAAGGTAAATCTTAAAACA
AACCAGTTTACCCATCTACCTGTAAGTAATATTTTGGATGATCTTGATTTACTAACCCAGATTGACCTTGAG
GATAACCCCTGGGACTGCTCCTGTGACCTGGTTGGACTGCAGCAATGGATACAAAAGTTAAGCAAGAACACA
GTGACAGATGACATCCTCTGCACTTCCCCCGGGCATCTCGACAAAAAGGAATTGAAAGCCCTAAATAGTGAA
ATTCTCTGTCCAGGTTTAGTAAATAACCCATCCATGCCAACACAGACTAGTTACCTTATGGTCACCACTCCT
GCAACAACAACAAATACGGCTGATACTATTTTACGATCTCTTACGGACGCTGTGCCACTGTCTGTTCTAATA
TTGGGACTTCTGATTATCTTCATCACTATTGTTTTCTGTGCTGCAGGGATAGTGGTTCTTGTTCTTCACCGC
AGGAGAAGATACAAAAAGAAACAAGTAGATGAGCAAATGAGAGACAACAGTCCTGTGCATCTTCAGTACAGC
ATGTATGGCCATAAAACCACTCATCACACTACTGAAAGACCCTCTGCCTCACTCTATGAACAGCACATGGTG
AGCCCCATGGTTCATGTCTATAGAAGTCCATCCTTTGGTCCAAAGCATCTGGAAGAGGAAGAAGAGAGGAAT
GAGAAAGAAGCAAGTGATGCAAAACATCTCCAAAGAAGTCTTTTGGAACAGGAAAATCATTCACCACTCACA
GGGTCAAATATGAAATACAAAACCACGAACCAATCAACAGAATTTTTATCCTTCCAAGATGCCAGCTCATTG
TACAGAAACATTTTAGAAAAAGAAAGCGAACTTCAGCAACTGGGAATCACAGAATACCTAAGGAAAAACATT
GCTCAGCTCCAGCCTGATATGGAGGCACATTATCCTGGAGCCCACGAAGAGCTGAAGTTAATCGAAACATTA
ATGTACTCACGTCCAAGGAAGGTATTAGTGGAACAGACAAAAAATGAGTATTTTGAACTTAAAGCTAATTTA
CATGCTGAACCTGACTATTTAGAAGTCCTGGAGCAGCAAACATAGATGGAGAGTTTGAGGGCTTTCGCAGAA
ATGCTGTGATTCTGTTTTAAGTCCATACCTTGTAAATTAGTGCCTTACGTGAGTGTGTCATCCATCAGAACC
TAAGCACAGCAGTAAACTATGGAGAAAAAA

In a search of public sequence databases, the NOV1a nucleic acid sequence, located on chromsome 13 has 1173 of 1932 bases (61%) identical to a KIAA0848 mRNA from human (gb:GENBANK-ID:AB020655|acc:AB020655). Public nucleotide databases include all GenBank databases and the GeneSeq patent database.

In all BLAST alignments herein, the “E-value” or “Expect” value is a numeric indication of the probability that the aligned sequences could have achieved their similarity to the BLAST query sequence by chance alone, within the database that was searched. For example, the probability that the subject (“Sbjct”) retrieved from the NOV1 BLAST analysis, e.g., thioredoxin mRNA from Ovis aries, matched the Query NOV1 sequence purely by chance is 7.4e−⁶⁸. The Expect value (E) is a parameter that describes the number of hits one can “expect” to see just by-chance when searching a database of a particular size. It decreases exponentially with the Score (S) that is assigned to a match between two sequences. Essentially, the E value describes the random background noise that exists for matches between sequences.

The Expect value is used as a convenient way to create a significance threshold for reporting results. The default value used for blasting is typically set to 0.0001. In BLAST 2.0, the Expect value is also used instead of the P value (probability) to report the significance of matches. For example, an E value of one assigned to a hit can be interpreted as meaning that in a database of the current size one might expect to see one match with a similar score simply by chance. An E value of zero means that one would not expect to see any matches with a similar score simply by chance. See, e.g., http://www.ncbi.nlm.nih.gov/Education/BLASTinfo/. Occasionally, a string of X's or N's will result from a BLAST search. This is a result of automatic filtering of the query for low-complexity sequence that is performed to prevent artifactual hits. The filter substitutes any low-complexity sequence that it finds with the letter “N” in nucleotide sequence (e.g., “NNNNNNNNNNNNN”) or the letter “X” in protein sequences (e.g., “XXXXXXXXX”). Low-complexity regions can result in high scores that reflect compositional bias rather than significant position-by-position alignment. (Wootton and Federhen, Methods Enzymol 266:554-571, 1996).

The disclosed NOV1a polypeptide (SEQ ID NO:2) encoded by SEQ ID NO:1 has 841 amino acid residues and is presented in Table 1B using the one-letter amino acid code. Signal P, Psort and/or Hydropathy results predict that NOV1a has a signal peptide and is likely to be localized in the plasma membrane with a certainty of 0.4600. In other embodiments, NOV1a may also be localized to the endoplasmic reticulum (membrane) with acertainty of 0.1000, the endoplasmic reticulum (membrane) with a certainty of 0.1000, or extracellularly with a certainty of 0.1000. The most likely cleavage site for a NOV1a peptide is between amino acids 20 and 21, at: LHS-QT.

SNP data for NOV1a can be found below in Example 3. SAGE data can also be found below for NOV1a in Example 4.

TABLE 1B
Encoded NOV1a protein sequence (SEQ ID NO: 2).

MKLWIHLFYSSLLACISLHSQTPVLSSRGSCDSLCNCEEKDGTMLIMCEAKGIKMVSEISVPPS
RPFQLSLLNNGLTMLHTNDFSGLTNAISIHLGFNNIADIEIGAFNGLGLLKQLHINHNSLEILK
EDTFHGLENLEFLQADNNFITVIEPSAFSKLNRLKVLILNDNAIESLPPNIFRFVPLTHLDLRG
NQLQTLPYVGFLEHIGRILDLQLEDNKWACNCDLLQLKTWLENMPPQSIIGDVVCNSPPFFKGS
ILSRLKKESICPTPPVYEEHEDPSGSLHLAATSSINDSRMSTKTTSILKLPTKAPGLIPYITKP
STQLPGPYCPIPCNCKVLSPSGLLIHCQERNIESLSDLRPPPQNPRKLILAGNIIHSLMKSDLV
EYFTLEMLHLGNNRIEVLEEGSFNNLTRLQKLYLNGNHLTKLSKGMFLGLHNLEYLYLEYNAIK
EILPGTFNPMPKLKVLYLNNNLLGVLPPHIFSGVPLTKVNLKTNQFTHLPVSNILDDLDLLTQI
DLEDNPWDCSCDLVGLQQWIQKLSKNTVTDDILCTSPGHLDKKELKALNSEILCPGLVNNPSMP
TQTSYLMVTTPATTTNTADTILRSLTDAVPLSVLILGLLIMFITIVFCAAGIVVLVLHRRRRYK
KKQVDEQMRDNSPVHLQYSMYGHKTTHHTTERPSASLYEQHMVSPMVHVYRSPSFGPKHLEEEE
ERNEKEGSDAKHLQRSLLEQENHSPLTGSNMKYKTTNQSTEFLSFQDASSLYRNILEKERELGQ
LGITEYLRNIAQLQPDMEAHYPGAHEELKLMETLMYSRPRKVLVEQTTKNEYFELKANLHAEPD
YLEVLEQQT

A search of sequence databases reveals that the NOV1a amino acid sequence has 266 of 543 amino acid residues (49%) identical to, and 337 of 543 amino acid residues (62%) similar to the 977 amino acid residue KLAA0848 protein from human (SPTREMBL-ACC:094933) (E=1.6e−¹⁶⁵), and 350 of 841 amino acid residues (41%) identical to, and 511 of 841 amino acid residues (60%) similar to the 845 amino acid residue Human gene 1 encoded secreted protein HMIAJ30 (patp:AAE01232) (E=1.6e−¹⁵⁶). Public amino acid databases include the GenBank databases, SwissProt, PDB and PIRP

NOV1a is expressed in at least the following tissues: breast, heart, bladder, colon, prostate, brain, lung and uterus. TaqMan expression data for NOV1a is shown below in Example 2.

NOV1b

A disclosed NOV1b nucleic acid of 2526 nucleotides (also referred to as AL356413.6) encoding a novel Insulin Like Growth Factor Binding Protein Complex-Acid Labile Subunit-like protein is shown in Table 1C. An open reading frame was identified beginning with an ATG initiation codon at nucleotides 1-3 and ending with a TAG codon at nucleotides 2524-2526. A putative untranslated region upstream from the initiation codon is underlined in Table 1C. The start and stop codons are in bold letters.

TABLE 1C
NOV1b nucleotide sequence (SEQ ID NO: 3).

ATGAAGCTGTGGATTCATCTCTTTTATTCATCTCTCCTTGCCTGTATATCTTTACACTCCCAAAC
TCCAGTGCTCTCATCCAGAGGCTCTTGTGATTCTCTTTGCAATTGTGAGGAAAAAGATGGCACAA
TGCTAATAAATTGTGAAGCAAAAGGTATCAAGATGGTATCTGAAATAAGTGTGCCACCATCACGA
CCTTTCCAACTAAGCTTATTAAATAACGGCTTGACGATGCTTCACACAAATGACTTTTCTGGGCT
TACCAATGCTATTTCAATACACCTTGGATTTAACAATATTGCAGATATTGAGATAGGTGCATTTA
ATGGCCTTGGCCTCCTGAAACAACTTCATATCAATCACAATTCTTTAGAAATTCTTAAAGAGGAT
ACTTTCCATGGACTGGAAAACCTGGAATTCCTGCAAGCAGATAACAATTTTATCACAGTGATTGA
ACCAAGTGCCTTTAGCAAGCTCAACAGACTCAAAGTGTTAATTTTAAATGACAATGCTATTGAGA
GTCTTCCTCCAAACATCTTCCGATTTGTTCCTTTAACCCATCTAGATCTTCGTGGAAATCAATTA
CAAACATTGCCTTATGTTGGTTTTCTCGAACACATTGGCCGAATATTGGATCTTCAGTTGGAGGA
CAACAAATGGGCCTGCAATTGTGACTTATTGCAGTTAAAAACTTGGTTGGAGAACATGCCTCCAC
AGTCTATAATTGGTGATGTTGTCTGCAACAGCCCTCCATTTTTTAAAGGAAGTATACTCAGTAGA
CTAAAGAAGGAATCTATTTGCCCTACTCCACCAGTGTATGAAGAACATGAGGATCCTTCAGGATC
ATTACATCTGGCAGCAACATCTTCAATAAATGATAGTCGCATGTCAACTAAGACCACGTCCATTC
TAAAACTACCCACCAAAGCACCAGGTTTGATACCTTATATTACAAAGCCATCCACTCAACTTCCA
GGACCTTACTGCCCTATTCCTTGTAACTGCAAAGTCCTATCCCCATCAGGACTTCTAATACATTG
TCAGGAGCGCAACATTGAAAGCTTATCAGATCTGAGACCTCCTCCGCAAAATCCTAGAAAGCTCA
TTCTAGCGGGAAATATTATTCACAGTTTAATGAAGTCTGATCTAGTGGAATATTTCACTTTGGAA
ATGCTTCACTTGGGAAACAATCGTATTGAAGTTCTTGAAGAAGGATCGTTTATGAACCTAACGAG
ATTACAAAAACTCTATCTAAATGGTAACCACCTGACCAAATTAAGTAAAGGCATGTTCCTTGGTC
TCCATAATCTTGAATACTTATATCTTGAATACAATGCCATTAAGGAAATACTGCCAGGAACCTTT
AATCCAATGCCTAAACTTAAAGTCCTGTATTTAAATAACAACCTCCTCCAAGTTTTACCACCACA
TATTTTTTCAGGGGTTCCTCTAACTAAGGTAAATCTTAAAACAAACCAGTTTACCCATCTACCTG
TAAGTAATATTTTGGATGATCTTGATTTGCTAACCCAGATTGACCTTGAGGATAACCCCTGGGAC
TGCTCCTGTGACCTGGTTGGACTGCAGCAATGGATACAAAAGTTAAGCAAGAACACAGTGACAGA
TGACATCCTCTGCACTTCCCCCGGGCATCTCGACAAAAAGGAATTGAAAGCCCTAAATAGTGAAA
TTCTCTGTCCAGGTTTAGTAAATAACCCATCCATGCCAACACAGACTAGTTACCTTATGGTCACC
ACTCCTGCAACAACAACAAATACGGCTGATACTATTTTACGATCTCTTACGGACGCTGTGCCACT
GTCTGTTCTAATATTGGGACTTCTGATTATGTTCATCACTATTGTTTTCTGTGCTGCAGGGATAG
TGGTTCTTGTTCTTCACCGCAGGAGAAGATACAAAAAGAAACAAGTAGATGAGCAAATGAGAGAC
AACAGTCCTGTGCATCTTCAGTACAGCATGTATGGCCATAAAACCACTCATCACACTACTGAAAG
ACCCTCTGCCTCACTCTATGAACAGCACATGGTGAGCCCCATGGTTCATGTCTATAGAAGTCCAT
CCTTTGGTCCAAAGCATCTGGAAGAGGAAGAAGAGAGGAATGAGAAAGAAGGAAGTGATGCAAAA
CATCTCCAAAGAAGTCTTTTGGAACAGGAAAATCATTCACCACTCACAGGGTCAAATATGAAATA
CAAAACCACGAACCAATCAACAGAATTTTTATCCTTCCAAGATGCCAGCTCATTGTACAGAAACA
TTTTAGAAAAAGAAAGGGAACTTCAGCAACTGGGAATCACAGAATACCTAAGGAAAAACATTGCT
CAGCTCCAGCCTGATATGGAGGCACATTATCCTGGAGCCCACGAAGAGCTGAAGTTAATGGAAAC
ATTAATGTACTCACGTCCAAGGAAGGTATTAGTGGAACAGACAAAAAATGAGTATTTTGAACTTA
AAGCTAATTTACATGCTGAACCTGACTATTTAGAAGTCCTGGAGCAGCAAACATAG

The disclosed NOV1b nucleotide encodes the NOV1a protein sequence disclosed above in Table 1B.

NOV1c

In the present invention, the target sequence identified previously, NOV1b, was subjected to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences or sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported below, which is designated NOV1c (also referred to as Accession Number CG52997-02). This is a mature protein with 100% identity to the previously identified sequence NOV1b.

A disclosed NOV1c nucleic acid of 2531 nucleotides (also referred to as CG52997-02) encoding a novel Insulin Like Growth Factor Binding Protein Complex-Acid Labile Subunit-like protein is shown in Table 1D. An open reading frame was identified beginning with a GAT initiation codon at nucleotides 24 and ending with a TAA codon at nucleotides 2513-2515. A putative untranslated region upstream from the initiation codon and downstream from the termination codon is underlined in Table 1D. The start and stop codons are in bold letters. Because the start codon is not a traditional ATG start codon, the reading frame shown below for NOV1c could be a partial reading frame that extends further in the 5′ direction.

TABLE 1D
NOV1c nucleotide sequence (SEQ ID NO: 4).

GGATTCTCTCTTTTATTCATCTCTCCTTGCCTGTATATCTTTACACTCCCAAACTCCAGTGCTCTCATCCAG
ACGCTCTTGTGATTCTCTTTGCAATTGTGAGGAAAAAGATGGCACAATGCTAATAAATTGTGAAGCAAAAGG
TATCAAGATGGTATCTGAAATAAGTGTGCTACCATCACGACCTTTCCAACTAAGCTTATTAAATAACGGCTT
GACGATGCTTCACACAAATGACTTTTCTGGGCTTACCAATGCTATTTCAATACACCTTGGATTTAACAATAT
TGCAGATATTGAGATAGGTGCATTTAATGGCCTTGGCCTCCTGAAACAACTTCATATCAATCACAATTCTTT
AGAAATTCTTAAAGAGGATACTTTCCATGGACTGGAAAACCTGGAATTCCTGCAAGCAGATAACAATTTTAT
CACAGTGATTGAACCAAGTGCCTTTAGCAAGCTCAACAGACTCAAAGTGTTAATTTTAAATGACAATGCTAT
TGAGACTCTTCCTCCAAACATCTTCCGATTTCTTCCTTTAACCCATCTAGATCTTCGTGGAAATCAATTACA
AACATTGCCTTATGTTGGTTTTCTCGAACACATTGGCCGAATATTGGATCTTCAGTTGGAGGACAACAAATG
GGCCTGCAATTGTGACTTATTGCAGTTAAAAACTTGGTTGGAGAACATGCCTCCACAGTCTATAATTGGTGA
TGTTCTCTGCAACAGCCCTCCATTTTTTAAAGGAAGTATACTCAGTAGACTAAAGAAGGAATCTATTTGCCC
TACTCCACCAGTGTATGAAGAACATGAGGATCCTTCAGGATCATTACATCTGGCAGCAACATCTTCAATAAA
TGATAGTCGCATGTCAACTAAGACCACGTCCATTCTAAAACTACCCACCAAAGCACCAGGTTTGATACCTTA
TATTACAAAGCCATCCACTCAACTTCCAGGACCTTACTGCCCTATTCCTTGTAACTGCAAAGTCCTATCCCC
ATCAGGACTTCTAATACATTGTCAGGAGCGCAACATTGAAAGCTTATCAGATCTGAGACCTCCTCCGCAAAA
TCCTAGAAAGCTCATTCTAGCGGGAAATATTATTCACAGTTTAATGAAGTCTGATCTAGTGGAATATTTCAC
TTTGGAAATGCTTCACTTGGGAAACAATCGTATTGAAGTTCTTGAAGAAGGATCGTTTATGAACCTAACGAG
ATTACAAAAACTCTATCTAAATGGTAACCACCTGACCAAATTAAGTAAAGGCATGTTCCTTGGTCTCCATAA
TCTTGAATACTTATATCTTGAATACAATGCCATTAAGGAAATACTGCCAGGAACCTTTAATCCAATGCCTAA
ACTTAAAGTCCTGTATTTAAATAACAACCTCCTCCAAGTTTTACCACCACATATTTTTTCAGGGGTTCCTCT
AACTAAGGTAAATCTTAAAACAAACCAGTTTACCCATCTACCTGTAAGTAATATTTTGGATGATCTTGATTT
GCTAACCCAGATTGACCTTGAGGATAACCCCTGGGACTGCTCCTGTGACCTGGTTGGACTGCAGCAATGGAT
ACAAAAGTTAAGCAAGAACACAGTGACAGATGACATCCTCTGCACTTCCCCCGGGCATCTCGACAAAAAGGA
ATTGAAAGCCCTAAATAGTGAAATTCTCTGTCCAGGTTTAGTAAATAACCCATCCATGGCAACACAGACTAG
TTACCTTATGGTCACCACTCCTGCAACAACAACAAATACGGCTGATACTATTTTACGATCTCTTACGGACGC
TGTGCCACTGTCTGTTCTAATATTGGGACTTCTGATTATGTTCATCACTATTGTTTTCTGTGCTGCAGGGAT
AGTGGTTCTTGTTCTTCACCGCAGGAGAAGATACAAAAAGAAACAAGTAGATGAGCAAATGAGAGACAACAG
TCCTGTGCATCTTCAGTACAGCATGTATGGCCATAAAACCACTCATCACACTACTGAAAGACCCTCTGCCTC
ACTCTATGAACAGCACATGGTGAGCCCCATGGTTCATGTCTATAGAAGTCCATCCTTTGGTCCAAAGCATCT
GGAAGAGGAAGAAGAGAGGAATGAGAAAGAAGGAAGTGATCCAAAACATCTCCAAAGAAGTCTTTTGGAACA
GGAAAATCATTCACCACTCACAGGGTCAAATATGAAATACAAAACCACGAACCAATCAACAGAATTTTTATC
CTTCCAAGATGCCAGCTCATTGTACAGAAACATTTTAGAAAAAGAAAGGGAACTTCAGCAACTGGGAATCAC
AGAATACCTAAGGAAAAACATTGCTCAGCTCCAGCCTGATATGGAGGCACATTATCCTGGAGCCCACGAAGA
GCTGAAGTTAATCGAAACATTAATGTACTCACGTCCAAGGAAGGTATTAGTGGAACAGACAAAAAATGAGTA
TTTTGAACTTAAAGCTAATTTACATGCTGAACCTGACTATTTAGAAGTCCTGGAGCAGCAAACATAAGCGCG
AATTCTGCTGT

In a search of public sequence databases, the NOV1c nucleic acid sequence, located on chromsome 13 has 2471 of 2480 bases (99%) identical to a gb:GENBANK-ID:AK026427|acc:AK026427.1 mRNA from Homo sapiens (Homo sapiens cDNA: FLJ22774 fis, clone KAIA1575) (E=0.0). Public nucleotide databases include all GenBank databases and the GeneSeq patent database.

The disclosed NOV1c polypeptide (SEQ ID NO:5) encoded by SEQ ID NO:4 has 837 amino acid residues and is presented in Table 1E using the one-letter amino acid code. Signal P, Psort and/or Hydropathy results predict that NOV1c has no signal peptide and is likely to be localized in the plasma membrane with a certainty of 0.4600. In other embodiments, NOV1c may also be localized to the endoplasmic reticulum (membrane) with a certainty of 0.1000, the endoplasmic reticulum (membrane) with a certainty of 0.1000, or extracellularly with a certainty of 0.1000.

TABLE 1E
Encoded NOV1c protein sequence (SEQ ID NO: 5).

DSLFYSSLLACISLHSQTPVLSSRGSCDSLCNCEEKDGTMLINCEAKGIKMVSEISVLPSRPFQLSLLNNGL
TMLHTNDFSGLTNAISIHLGFNNIADIEIGAFNGLGLLKQLHINHNSLEILKEDTFHGLENLEFLQADNNFI
TVIEPSAFSKLNRLKVLILNDNAIESLPPNIFRFVPLTHLDLRGNQLQTLPYVGFLEHIGRILDLQLEDNKW
ACNCDLLQLKTWLENNPPQSIIGDVVCNSPPFFKGSILSRLKKESICPTPPVYEEHEDPSGSLHLAATSSIN
DSRMSTKTTSILKLPTKAPGLIPYITKPSTQLPGPYCPIPCNCKVLSPSGLLIHCQERNIESLSDLRPPPQN
PRKLILAGNIIHSLMKSDLVEYFTLEMLHLGNNRIEVLEEGSFMNLTRLQKLYLNGNHLTKLSKGMFLGLHN
LEYLYLEYNAIKEILPGTFNPMPKLKVLYLNNNLLQVLPPHIFSGVPLTKVNLKTNQFTHLPVSNILDDLDL
LTQIDLEDNPWDCSCDLVGLQQWIQKLSKNTVTDDILCTSPGHLDKKELKALNSEILCPGLVNNPSMPTQTS
YLMVTTPATTTNTADTILRSLTDAVPLSVLILGLLIMFITIVFCAAGIVVLVLHRRRRYKKKQVDEQMRNNS
PVHLQYSMYGHKTTHHTTERPSASLYEQHNVSPMVHVYRSPSFGPKHLEEEEERNEKEGSDAKHLQRSLLEQ
ENHSPLTGSNMKYKTTNQSTEFLSFQDASSLYRNILEKERELQQLGITEYLRKNIAQLQPDMEAHYPGAHEE
LKLMETLMYSRPRKVLVEQTKNEYFELKANLHAEPDYLEVLEQQT

A search of public sequence databases reveals that the NOV1c amino acid sequence has 427 of 436 amino acid residues (97%) identical to, and 428 of 436 amino acid residues (98%) similar to, the 440 amino acid residue ptnr:SPTREMBL-ACC:Q9H5Y7 protein from Homo sapiens (Human) (CDNA: FLJ22774 FIS, CLONE KAIA1575) (E=5.7e−²³⁰). Public amino acid databases include the GenBank databases, SwissProt, PDB and PIR.

NOV 1c is expressed in at least the following tissues: Heart, Coronary Artery, Pancreas, Small Intestine, Peripheral Blood, Brain, Mammary gland/Breast, Uterus, Vulva, Prostate, Lung, Trachea, Skin, Colon. Expression information was derived from the tissue sources of the sequences that were included in the derivation of the sequence of NOV1c.

The proteins encoded by the NOV1a, 1b and 1c nucleotides are very closely homologous as is shown in the alignment in Table 1F. As shown, the sequences encoded by the NOV1a and 1b nucleic acid sequences are 100% identical.

[image]

[image]

Homologies to any of the above NOV1 proteins will be shared by the other two NOV1 proteins insofar as they are homologous to each other as shown above. Any reference to NOV1 is assumed to refer to all three of the NOV1 proteins in general, unless otherwise noted.

The disclosed NOV1a polypeptide has homology to the amino acid sequences shown in the BLASTP data listed in Table 1G.

TABLE 1G
BLAST results for NOV1a

| Gene Index/ | | Length | Identity | Positives | |
|---|---|---|---|---|---|
| Identifier | Protein/Organism | (aa) | (%) | (%) | Expect |
| gi\|14758126\|ref\|XP_{—} | hypothetical | 798 | 798/798 | 798/798 | 0.0 |
| 033182.1\| | protein FLJ22774 | | (100%) | (100%) | |
| | [Homo sapiens] | | | | |
| gi\|14149932\|ref\|NP_{—} | hypothetical | 440 | 425/425 | 425/425 | 0.0 |
| 115605.1\| | protein FLJ22774 | | (100%) | (100%) | |
| | [Homo sapiens] | | | | |
| gi\|6691962\|emb\|CAB65788.1\| | bG256O22.1 | 853 | 354/866 | 504/866 | e-161 |
| | (similar to | | (40%) | (57%) | |
| | IGFALS (insulin- | | | | |
| | like growth | | | | |
| | factor binding | | | | |
| | protein, acid | | | | |
| | labile subunit)) | | | | |
| | [Homo sapiens] | | | | |
| gi\|11360190\|pir\|\|T46279 | hypothetical | 314 | 314/314 | 314/314 | e-160 |
| | protein | | (100%) | (100%) | |
| | DKFZp564O1278.1 - | | | | |
| | human (fragment) | | | | |
| gi\|14424224\|sp\|O94991\| | HYPOTHETICAL | 966 | 356/915 | 534/915 | e-158 |
| Y918_HUMAN | PROTEIN KIAA0918 | | (38%) | (57%) | |

The homology between these and other sequences is shown graphically in the ClustalW analysis shown in Table 1H. In the ClustalW alignment of the NOV1 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

[image]

[image]

The presence of identifiable domains in NOV1, as well as all other NOVX proteins, was determined by searches using software algorithms such as PROSITE, DOMAIN, Blocks, Pfam, ProDomain, and Prints, and then determining the Interpro number by crossing the domain match (or numbers) using the Interpro website (http:www.ebi.ac.uk/ interpro). DOMAIN results for NOV1 as disclosed in Tables 1I-IL, were collected from the Conserved Domain Database (CDD) with Reverse Position Specific BLAST analyses. This BLAST analysis software samples domains found in the Smart and Pfam collections. For Table 1E and all successive DOMAIN sequence alignments, fully conserved single residues are indicated by black shading or by the sign (|) and “strong” semi-conserved residues are indicated by grey shading or by the sign (+). The “strong” group of conserved amino acid residues may be any one of the following groups of amino acids: STA, NEQK, NHQK, NDEQ, QHRK, MILV, MILF, HY, FYW.

Tables 1I-1L lists the domain description from DOMAIN analysis results against NOV1. This indicates that the NOV1 sequence has properties similar to those of other proteins known to contain this domain.

TABLE 11

Domain Analysis of NOV1
gnl|Smart|smart00082, LRRCT, Leucine rich repeat C-terminal
domain.
(SEQ ID NO: 65)
CD-Length = 51 residues, 100.0% aligned
Score = 49.7 bits (117), Expect = 7e−07

| | | | |
|---|---|---|---|
| Query: | 517 | NPWDCSCDLVGLQQWIQKLSKNTVTDDILCTSPGHLDKKELKALNSEILCP | 567 |
| | | \|\|+ \| \|+\| \| +\|+\| \|+ \| \|\| \| \| \| \| \|\| | |
| Sbjct: | 1 | NPFICDCELRWLLRWLQANRHLQDPVDLRCASPESLRGPLLLLLPSSFKCP | 51 |

TABLE 1J

Domain Analysis of NOV1
gnl|Smart|smart00082, LRRCT, Leucine rich repeat C-terminal
domain.
(SEQ ID NO: 65)
CD-Length = 51 residues, 100.0% aligned
Score = 45.1 bits (105), Expect = 2e−05

| | | | |
|---|---|---|---|
| Query: | 218 | NKWACNCDLLQLKTWLENMPPQSIIGDVVCNSPPFFKGSILSRLKXESICP | 268 |
| | | \| + \|+\|+\| \| \|\|+ \|+ \| \|\| +\| +\| \| \|\| | |
| Sbjct: | 1 | NPFICDCELRWLLRWLQANRHLQDPVDLRCASPESLRGPLLLLLPSSFKCP | 51 |

TABLE 1K

Domain Analysis of NOV1
gnl|Pfam|pfam01463, LRRCT, Leucine rich repeat C-terminal
domain.
Leucine Rich Repeats pfam00560 are short sequence motifs
present in a number of proteins with diverse functions and
cellular locations.
Leucine Rich Repeats are often flanked by cysteine rich do-
mains. This domain is often found at the C-terminus of tandem
leucine rich repeats
(SEQ ID NO: 66)
CD-Length = 51 residues, 100.0% aligned
Score = 47.8 bits (112), Expect = 3e−06

| | | | |
|---|---|---|---|
| Query: | 517 | NPWDCSCDLVGLQQWIQKLSKNTVTDDILCTSPGHLDKKELKALNSEILCP | 567 |
| | | \|\|+ \| \|+\| \| +\|+++ + +\|+ \| \|\| \| \|+ \| \|+ \|\| | |
| Sbjct: | 1 | NPFICDCELRWLLRWLREPRRLEDPEDLRCASPESLRGPLLELLPSDFSCP | 51 |

TABLE 1L

Domain Analysis of NOV1
gnl|Pfam|pfam01463, LRRCT, Leucine rich repeat C-terminal
domain.
Leucine Rich Repeats pfam00560 are short sequence motifs
present in a number of proteins with diverse functions and
cellular locations.
Leucine Rich Repeats are often flanked by cysteine rich do-
mains. This domain is often found at the C-terminus of tandem
leucine rich repeats.
(SEQ ID NO: 66)
CD-Length = 51 residues, 100.0% aligned
Score = 46.2 bits (108), Expect = 7e−06

| | | | |
|---|---|---|---|
| Query: | 218 | NKWACNCDLLQLKTWLENMPPQSIIGDVVCNSPPFFKGSILSRLKKESICP | 268 |
| | | \| + \|+\|+\| \| \|\| \|+ \| \|\| +\| +\| \| + \|\| | |
| Sbjct: | 1 | NPFICDCELRWLLRWLREPRRLEDPEDLRCASPESLRGPLLELLPSDFSCP | 51 |

Proteins belonging to the IGFBP-ALS family of proteins play an important role in regulating the levels of circulating hormones. The acid labile subunit of the complex plays an important role in regulating the stability of the complex and ensuring high levels of circulating hormones that are regulated by the IGFBP family of proteins. This protein also has a leucine rich repeat that is a common domain in many proteins that are important for the developing embryo. As a result this protein may play an important role in development and disease.

Insulin-like growth factors (IGFs) I and II are important regulators of cell proliferation and differentiation (Ueki I et al., Proc Natl Acad Sci USA 2000 Jun. 6;97(12):6868-73). After birth, plasma IGFs, representing mostly liver-derived IGFs, circulate in ternary complexes of 150 kDa consisting of one molecule each of IGF, IGF-binding protein (IGFBP) 3, and an acid labile subunit (ALS). Onset of ALS synthesis after birth is the primary factor driving the formation of ternary complexes. Capture of IGFs by ALS is thought to allow the development of a plasma reservoir without negative effects such as hypoglycemia and cell proliferation. To evaluate the importance of ALS and ternary complexes, we have created mice in which the ALS gene has been inactivated. The mutation was inherited in a Mendelian manner, without any effects on survival rates and birth weights. A growth deficit was observed in null mice after 3 weeks of life and reached 13% by 10 weeks. This modest phenotype was observed despite reductions of 62 and 88% in the concentrations of plasma IGF-I and IGFBP-3, respectively. Increased turnover accounted for these reductions because indices of synthesis in liver and kidney were not decreased. Surprisingly, absence of ALS did not affect glucose and insulin homeostasis. Therefore, ALS is required for postnatal accumulation of IGF-I and IGFBP-3 but, consistent with findings supporting a predominant role for locally produced IGF-I, is not critical for growth. This model should be useful to determine whether presence of ALS is needed for other actions of liver-derived IGF-I and for maintenance of homeostasis in presence of high circulating levels of IGF-II.

In circulation, insulin-like growth factor-I (IGF-D) is bound in a trimeric complex of 150 kDa with IGF binding protein-3 (IGFBP-3) and the acid-labile subunit (ALS). (Moller S et al., J Hepatol March 2000;32(3):441-6). Whereas circulating IGF-I and IGFBP-3 are reported to be low in patients with chronic liver failure, the level of ALS has not been described in relation to hepatic dysfunction. The aim of the present study was therefore to measure circulating and hepatic venous concentrations of ALS in relation to hepatic function and the IGF axis.

The insulin-like growth factor (IGF) binding proteins (IGFBPs) were initially identified as carrier proteins for IGF-I and IGF-II in a variety of biologic fluids. (Rosenfeld R G et al., Pediatrics October 1999;104(4 Pt 2):1018-21). Their presumed function was to protect IGF peptides from degradation and clearance, increase the half-life of the IGFs, and deliver them to appropriate tissue receptors. The concept of IGFBPs as simple carrier proteins has been complicated, however, by a number of observations: 1) the six IGFBPs vary in their tissue expression and their regulation by other hormones and growth factors; 2) the IGFBPs are subjected to proteolytic degradation, thereby altering their affinities for the IGFs; 3) IGFBP-3 and IGFBP-5, in addition to binding IGFs, also can associate with an acid-labile subunit, thereby increasing further the half-life of the IGFs; 4) in addition to modifying the access of IGF peptides to IGF and insulin receptors, several of the IGFBPs may be capable of increasing IGF action; 5) some of the IGFBPs may be capable of IGF-independent regulation of cell growth; 6) some of the IGFBPs are associated with cell membranes or possibly with membrane receptors; and 7) some of the IGFBPs have nuclear recognition sites and may be found within the nucleus. Additionally, a number of cDNAs identified recently have been found to encode proteins that bind IGFs, but with substantially lower affinities than is the case with IGFBPs. The N-terminal regions of the predicted proteins are structurally homologous to the classic IGFBPs, with conservation of the cysteine-rich region. These observations suggest that these low-affinity binders are members of an IGFBP superfamily, capable of regulating cell growth by both IGF-dependent and IGF-independent mechanisms insulin-like growth factor, insulin-like growth factor binding proteins.

Total IGF-I level in serum is a sensitive index during growth hormone (GH) replacement therapy of adults, since GH stimulates the hepatic expressions of both insulin-like growth factor (IGF-I) and acid-labile subunit (ALS) and the major part of IGF-I in the circulation is found in a ternary complex together with ALS and IGFBP-3. (Hall K et al., J Endocrinol Invest 1999;22(5 Suppl):48-57) However, other regulators of the proteins constituting the ternary complex may influence IGF-I levels. In healthy subjects the serum IGF-I levels are low at birth, rise during childhood, with peak levels during puberty, and decline with increasing age. This pattern has been attributed to the age-dependent GH production, but it is unknown whether the wide range of IGF-I levels within each age interval is due to GH production or GH sensitivity. In elderly twins approximately 60% of IGF-I levels are genetically determined. The remaining environmental dependency of IGF-I is partly due to nutrition. Both caloric and protein content of the diet is of importance. Thus, low IGF-I levels are found in GH deficient patients as well as in patients with GH resistance due to malnutrition or GH receptor defects. It is essential that IGF-I determination is performed by assays in which IGFBPs do not interfere, and that IGF-I concentration is evaluated in relation to age, i.e. expressed in SD score, and the number of individuals constituting the reference intervals improves the sensitivity and specificity. Although determination of IGF-I is recommended in assessing GH deficiency in children, its diagnostic value in patients with adult onset of GH deficiency is not agreed upon. In the age group above 40-80 years many patients with pituitary/hypothalamic disorders and GH peaks below 3 μg/l during provocation tests have normal IGF-I levels. It is not clarified, whether the IGF-I levels within normal range for age is due to endogenous basal GH production being sufficient or other factors stimulating IGF-I, IGFBP-3 or ALS expressions.

Circulating insulin-like growth factors (IGFs) represent an important pool of potential hypoglycemic activity, which is largely inhibited by their sequestration in a heterotrimeric complex comprising growth factor, IGF-binding protein-3 (IGFBP-3), and acid-labile subunit (ALS). (Baxter R C Metabolism October 1995;44(10 Suppl 4):12-7). Less than 1% of total IGFs circulate in the free form, yet even this amount might contribute significantly to circulating insulin-like activity. The ternary binding protein complex appears to inhibit insulin-like activity of bound IGFs by preventing their egress from the circulation. Although the integrity of this complex might be affected by limited proteolysis of IGFBP-3 in pregnancy and catabolic conditions, the evidence that this increases IGF bioavailability, and thus hypoglycemic potential, is as yet unclear. However, in patients with IGF-II-secreting tumors, hypoglycemia may result from a failure of the ternary complex to adequately sequester the IGFs. Improvement in complex formation, by treatment with corticosteroids or growth hormone, alleviates the hypoglycemia, even if (as seen with growth hormone treatment) IGF-II hypersecretion persists. In these patients, blood glucose levels are inversely correlated with IGFBP-2 levels, suggesting that this protein might play a part in transporting IGFs to their target tissues. Conversely, ALS levels correlate positively with blood glucose, emphasizing the importance of the ternary complex in preventing hypoglycemia. Unlike the other IGF-binding proteins, IGFBP-1 is acutely regulated in the circulation, in a manner consistent with its acting as a glucose counterregulator. It might act in this way by inhibiting the activity of free IGFs in the circulation.

Leucine-rich repeats (LRRs) are relatively short motifs (22-28 residues in length) found in a variety of cytoplasmic, membrane and extracellular proteins (InterPro). Although these proteins are associated with widely different functions, a common property involves protein-protein interaction. Little is known about the 3D structure of LRRs, although it is believed that they can form amphipathic structures with hydrophobic surfaces capable of interacting with membranes. In vitro studies of a synthetic LRR from Drosophila Toll protein have indicated that the peptides form gels by adopting beta-sheet structures that form extended filaments. These results are consistent with the idea that LRRs mediate protein-protein interactions and cellular adhesion. Other functions of LRR-containing proteins include, for example, binding to enzymes and vascular repair. The 3D structure of ribonuclease inhibitor, a protein containing 15 LRRs, has been determined, revealing LRRs to be a new class of alpha/beta fold. LRRs form elongated non-globular structures and are often flanked by cysteine rich domains.

The disclosed NOV1 nucleic acid of the invention encoding a Insulin Like Growth Factor Binding Protein Complex-Acid Labile Subunit-like protein includes the nucleic acid whose sequence is provided in Table 1A, C and E or a fragment thereof. The invention also includes a mutant or variant nucleic acid any of whose bases may be changed from the corresponding base shown in Table 1A, C, or E while still encoding a protein that maintains its Insulin Like Growth Factor Binding Protein Complex-Acid Labile Subunit-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 10% percent of the bases may be so changed.

The disclosed NOV1 protein of the invention includes the Insulin Like Growth Factor Binding Protein Complex-Acid Labile Subunit-like protein whose sequence is provided in Table 1B or 1E. The invention also includes a mutant or variant protein any of whose residues may be changed from the corresponding residue shown in Table 1B or 1E while still encoding a protein that maintains its Insulin Like Growth Factor Binding Protein Complex-Acid Labile Subunit-like activities and physiological functions, or a functional fragment thereof. In the mutant or variant protein, up to about 60% percent of the residues may be so changed.

The invention further encompasses antibodies and antibody fragments, such as F_{ab} or (F_{ab})_{2,} that bind immunospecifically to any of the proteins of the invention.

The above defined information for this invention suggests that this Insulin Like Growth Factor Binding Protein Complex-Acid Labile Subunit-like protein (NOV1) may function as a member of a “Insulin Like Growth Factor Binding Protein Complex-Acid Labile Subunit family”. Therefore, the NOV1 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV1 nucleic acids and proteins of the invention are useful in potential therapeutic applications implicated in cancer including but not limited to various pathologies and disorders as indicated below. For example, a cDNA encoding the Insulin Like Growth Factor Binding Protein Complex-Acid Labile Subunit-like protein (NOV1) may be useful in gene therapy, and the Insulin Like Growth Factor Binding Protein Complex-Acid Labile Subunit-like protein (NOV1) may be useful when administered to a subject in need thereof. By way of nonlimiting example, the compositions of the present invention will have efficacy for treatment of patients suffering from cancer, cystitis, incontinence, fertility, cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation recovery. The NOV1 nucleic acid encoding the Insulin Like Growth Factor Binding Protein Complex-Acid Labile Subunit-like protein of the invention, or fragments thereof, may further be useful in diagnostic applications, wherein the presence or amount of the nucleic acid or the protein are to be assessed.

NOV1 nucleic acids and polypeptides are further useful in the generation of antibodies that bind immuno-specifically to the novel NOV1 substances for use in therapeutic or diagnostic methods. These antibodies may be generated according to methods known in the art, using prediction from hydrophobicity charts, as described in the “Anti-NOVX Antibodies” section below. The disclosed NOV1 protein has multiple hydrophilic regions, each of which can be used as an immunogen. In one embodiment, a contemplated NOV1 epitope is from about amino acids 10 to 50. In another embodiment, a NOV1 epitope is from about amino acids 80 to 120. In additional embodiments, NOV1 epitopes are from about amino acids 180 to 220, from about amino acids 230 to 300, from about amino acid 330 to 350, from about amino acid 370 to 400, from about amino acid 480 to 540, from about amino acid 550 to 560, and from about amino acids 620 to 840. These novel proteins can be used in assay systems for functional analysis of various human disorders, which will help in understanding of pathology of the disease and development of new drug targets for various disorders.

NOV2

A disclosed NOV2 nucleic acid of 3609 nucleotides (also referred to as 101599929_EXT1) encoding a novel Attractin like protein is shown in Table 2A. An open reading frame was identified beginning with an ATG initiation codon at nucleotides 7-9 and ending with a TAA codon at nucleotides 3562-3564. A putative untranslated region upstream from the initiation codon and downstream from the termination codon is underlined in Table 2A, and the start and stop codons are in bold letters.

TABLE 2A
NOV2 nucleotide sequence (SEQ ID NO: 6).

CAGTGGATGCAGAAGGCAGACAGCAGCACCGAGACGATGAAGGAGAAGAGGACAGCGGCTGCGATCACCGTG
CGGCACAGGACCGGCTCCTGCTTCTCGGGCCGCTGTGTCAACTCCACCTGCCTCTGCGACCCGGGCTGGGTG
GGGGACCAGTGCCAGCACTGCCAGGGCAGGTTCAGGTTAACAGAACCTTCTGGATATTTAACAGATGGCCCA
ATTAACTATAAATATAAAACTAAATGTACTTGGCTCATTGAAGGCCCAAATGCAGTGTTAAGATTAAGATTC
AATCATTTTGCTACAGAATGTAGCTGGGATCATATGTATGTTTATGATGGAGATTCAATATATGCACCTTTA
ATAGCTTCTTTTAGTGGTTTGATAGTCCCTGAAATAAGGGGCAATGAAACTGTGCCTGAAGTTGTTACTACA
TCTGGCTATGCACTGTTACATTTTTTTAGTGATGCTGCGTATAATCTAACTGGTTTCAACATTTTCTATTCG
ATCAATTCTTGTCCTAACAATTGCTCTGGTCATGGGAAGTGTACAACTAGTGTCTCTGTTCCAAGTCAAGTA
TATTGTGAATGTGATAAATACTGGAAGGGTGAAGCTTGTGATATTCCTTACTGTAAAGCCAATTGCGGCAGT
CCAGATCACGGTTACTGTGACCTGACTGGAGAAAAATTATGTGTCTGCAATGATAGTTGGCAAGGTATAGGT
CCTGATTGTTCTTTGAATGTTCCCTCTACTGAGTCTTACTGGATTCTGCCAAACGTTAAACCCTTCAGTCCT
TCTGTAGGTCGGGCTTCACATAAAGCAGTTTTACACGGGAAATTTATGTGGGTGATTGGTGGATATACTTTT
AACTACAGTTCTTTTCAAATGGTCCTAAGTTACAATTTAGAAAGCAGTATATGGAATGTAGGAACTCCATCA
AGGGGACCTCTCCAGAGATATGGACACTCTCTTGCTTTATATCAGGAAAACATCTTTATGTATGGAGGCAGA
ATTGAAAGAAATGATGGCAATGTCACAGATGAATTATGGGTTTTTAACATACATAGTCAGTCATGGAGTACA
AAAACTCCTACTGTTCTTGGACATGGTCAGCAGTATGCTGTGGAGGGACATTCAGCACATATTATGGAGTTG
GATAGTAGAGATGTTGTCATGATCATAATATTTGGATATTCTGCAATATATGGTTATACAAGCAGCATACAG
GAATACCATATCTGTTCAAACACTTGGCTTGTTCCAGAAACTAAAGGAGCTATTGTACAAGGTGGATATGGC
CATACTAGTGTGTATGATGAAATAACAAAGTCCATTTATGTTCATGGAGGGTATAAAGCATTGCCAGGGAAC
AAATATGGATTGGTTGATGATCTTTATAAATATGAAGTTAACACTAAGACTTGGACTATTTTGAAAGAAAGT
GGGTTTGCCAGATACCTTCATTCAGCTGTTCTTATCAATGGAGCTATGCTTATTTTTGGAGGAAATACCCAT
AATGACACTTCCTTGAGTAACGGTGCAAAATGTTTTTCTGCCGATTTCCTGGCATATGACATATGCCCAGGC
TGGAGTGCAGTGGCACGATCTCAGCTCACTCCCACCTCCACCTCCCACGTTCAAGCGATTCTCAATAGGTCC
ATGTATATATTTGGGGGATTTTCTAGTGTACTCCTTAATGATATCCTTGTATACAAGCCTCCAAATTGCAAG
GCTTTCAGAGATGAAGAACTTTGTAAAAATGCTGGTCCAGGGATAAAATGTGTTTGGAATAAAAATCACTGT
GAATCTTGGGAATCTGGGAATACTAATAATATTCTTAGAGCAAAGTGCTTTTCTAAAAGAAATCTCTGCAGT
GACAGATGTTACAGATATGCAGATTGTGCCAGCTGTACTGCCAATACAAATGGGTGCCAATGGTGTGATGAC
AAGAAATGCATTTCGGCAAATAGTAACTGCAGTATGGTTAGTATTTTTGGGTATATAACCTTGCCTTCACAG
TTCCCATTCTATTATTGCTACAGATATGCAGATTGTGCCAGCTGTACTGCCAATACAAATGGGTGCCAATGG
TGTGATGACAAGAAATGCATTGCTTTACCAGCTCATCTTTGTGGAGAAGGATGGAGTCATATTGGGGATGCT
TGTCTTAGAGTCAATTCCAGTAGAGAAAACTATGACAATGCAAAACTTTATTGCTATAATCTTAGTGGAAAT
CTTGCTTCATTAACAACCTCAAAAGAAGTAGAATTTGTTCTGGATGAAATACAGAAGTATACACAACAGAAA
GTATCACCTTGGGTAGGCTTGCGCAAGATCAATATATCCTATTGCGGATGGGAAGACATGTCTCCTTTTACA
AACACAACACTACAGTGGCTTCCTGGCGAACCCAATGATTCTGGGTTTTGTGCATATCTGGAAAGGGCTGCA
GTGGCAGGCTTAAAAGCTAATCCTTGTACATCTATGGCAAATGGCCTTGTCTGTGAAAAACCTGTTAATCAA
AATGCGAGGCCGTGCAAAAAGCCATGCTCTCTGAGGACATCATGTTCCAACTGTACAAGCAATGGCATCGAG
TGTATGTGGTGCAGCAGTACGAAACGATGTGTTGACTCTAATGCCTATATCATCTCTTTTCCATATGGACAA
TCTCTAGAGTGGCAAACTGCCACCTGCTCCCGTGCTCAAAATTGTTCTGGATTGAGAACCTGTGGACAGTGT
TTGGAACAGCCTGAATGTGGCTGGTGCAATGATCCTAGTAATACAGGAAGAGGACATTGCATTGAAGGTTCT
TCACGGGGACCAATGAAGCTTATTGGAATGCACCACAGTGAGATGGTTCTTGACACCAATCTTTGCCCCAAA
GAAAAGAACTATGAGTGGTCCTTTATCCAGTGTCCAGCTTGCCAGTGTAATGGACATAGCACTTGCATCAAT
AATAATGTGTGCGAACAGTGTAAAAATCTCACCACAGGAAAGCAGTGTCAAGATTGTATGCCAGGTTATTAT
GGAGATCCAACCAATGGTGGACAGTGCACAGCTTGTACATGCAGTGGCCATGCAAATATCTGTCATCTGCAC
ACAGGAAAATGTTTCTGCACAACTAAAGGAATAAAAGGTGACCAATGCCAATTGTGTGACTCTGAAAATCGC
TATGTTGGTAATCCACTTAGAGGAACATGTTATTGTAAGTATAGCCTTTTGATTGATTATCAATTTACCTTC
AGCTTATTACAGGAAGATGATCGCCACCATACTGCCATAAACTTTATAGCAAACCCAGAACAGGTGAGGAAA
AATCTGGATATATCAATTAATGCATCAAACAACTTTAATCTCAACATTACGTGGTCTGTCGGTTCAGCTGGA
ACAATATCTGGGGAAGAGACTTCTATAGTTTCCAAGAATAATATAAAGGAATACAGAGATAGTTTTTCCTAT
GAAAAATTTAACTTTAGAAGCAATCCTAACATTACATTCTATGTGTACGTCAGCAACTTTTCCTGGCCTATT
AAAATACAGGTAAGTGTTAAGAGTATTTACTTCTAATGACCATAATATCATTAAGAAAAGAATCGTGCTTTT
GTCCAAAGT

The disclosed NOV2 nucleic acid sequence, localized to chromsome 10, has 494 of 694 bases (71%) identical to an Attractin protein mRNA from mouse (GENBANK-ID: AF119821) (E=2.9e⁻²⁰⁴).

A NOV2 polypeptide (SEQ ID NO:7) encoded by SEQ ID NO:6 has 1185 amino acid residues and is presented using the one-letter code in Table 2B. Signal P, Psort and/or Hydropathy results predict that NOV2 does not contain a signal peptide and is likely to be localized in the mitochondrial membrane space with a certainty of 0.3600. In other embodiments, NOV2 may also be localized to the microbody (peroxisome) with a certainty of 0.3000, or the lysosome (lumen) with a certainty of 0.1000.

TABLE 2B
Encoded NOV2 protein sequence (SEQ ID NO: 7).

MQKADSSTETMKEKRTAAAITVRHRTGSCFSGRCVNSTCLCDPGWVGDQCQHCQGRFRLTEPSGYLTDGPIN
YKYKTKCTWLIEGPNAVLRLRFNHFATECSWDHMYVYDGDSIYAPLIASFSGLIVPEIRGNETVPEVVTTSG
YALLHFFSDAAYNLTGFNIFYSINSCPNNCSGHGKCTTSVSVPSQVYCECDKYWKGEACDIPYCKANCGSPD
HGYCDLTGEKLCVCNDSWQGIGPDCSLNVPSTESYWILPNVKPFSPSVGRASHKAVLHGKFMNVIGGYTFNY
SSFQMVLSYNLESSIWNVGTPSRGPLQRYGHSLALYQENIFMYGGRIETNDGNVTDELWVFNIHSQSWSTKT
PTVLGHGQQYAVEGHSAHIMELDSRDVVMIIIFGYSAIYGYTSSIQEYHICSNTWLVPETKGAIVQGGYGHT
SVYDEITKSIYVHGGYKALPGNXYGLVDDLYKYEVNTKTWTILKESGFARYLHSAVLINGANLIFGGNTHND
TSLSNGAKCFSADFLAYDICPGWSAVARSQLTATSTSHVQAILNRSMYIFGGFSSVLLNDILVYKPPNCKAF
RDEELCKNAGPGIKCVWNKNHCESWESGNTNNILRAKCFSKRNLCSDRCYRYADCASCTANTNGCQWCDDKK
CISANSNCSMVSIFCYITLPSQFPFYYCYRYADCASCTANTNGCQWCDDKKCIALPAHLCGEGWSHIGDACL
RVNSSRENYDNAKLYCYNLSGNLASLTTSKEVEFVLDEIQKYTQQKVSPWVGLRKINISYWGWEDMSPFTNT
TLQWLPGEPNDSGFCAYLERAAVAGLKANPCTSMANGLVCEKPVNQNARPCKKPCSLRTSCSNCTSNGMECM
WCSSTKRCVDSNAYIISFPYGQCLEWQTATCSRAQNCSGLRTCGQCLEQPECGWCNDPSNTGRGHCIEGSSR
GPMKLIGMHHSEMVLDTNLCPKEKNYEWSFIQCPACQCNGHSTCINNNVCEQCKNLTTGKQCQDCMPGYYGD
PTNGGQCTACTCSGHANICHLHTGKCFCTTKGIKGDQCQLCDSENRYVGNPLRGTCYCKYSLLIDYQFTFSL
LQEDDRHHTAINFIANPEQVRKNLDISINASNNFNLNITWSVGSAGTISGEETSIVSKNNIKEYRDSFSYEK
FNFRSNPNITFYVYVSNFSWPIKIQVSVKSIYF

The disclosed NOV2 amino acid sequence has 703 of 1197 amino acid residues (58%) identical to, and 895 of l 197 amino acid residues (74%) similar to, the 1198 amino acid residue Attractin protein from human (SPTREMBL-ID:O75882) (E=0.0), and 703 of 1197 amino acid residues (58%) identical to, and 895 of 1 197 amino acid residues (74%) similar to, the 1198 amino acid residue human soluble Attractin-l protein (patp:AAY70689) (E=0.0).

NOV2 is expressed in at least the following tissues: Brain, Kidney, Muscle, Pancreas, Prostate, Uterus, Breast, Colon, Ovary, and Liver. In addition, the sequence is predicted to be expressed in the following tissues because of the expression pattern of (GENBANK-ID: AF119821) a closely related attractin homolog in mouse: Brain, Heart, Kidney, Liver, Lung, Skin, Spinal cord, and Pituitary.

NOV2 also has homology to the amino acid sequences shown in the BLASTP data listed in Table 2C.

TABLE 2C
BLAST results for NOV2

| Gene Index/ | Protein/ | Length | Identity | Positives | |
|---|---|---|---|---|---|
| Identifier | Organism | (aa) | (%) | (%) | Expect |
| gi\|13160051\|emb\|CAC32456.1\| | dJ741H3.1.1 | 1011 | 566/1015 | 739/1015 | 0.0 |
| | (attractin | | (55%) | (72%) | |
| | (with | | | | |
| | dipeptidylpeptidase | | | | |
| | IV | | | | |
| | activity) | | | | |
| | secreted | | | | |
| | isoform) | | | | |
| | [Homo | | | | |
| | sapiens] | | | | |
| gi\|4585307\|gb\|AAD25372.1\| | attractin | 1428 | 677/1174 | 865/1174 | 0.0 |
| AF119821_1 | [Mus | | (57%) | (73%) | |
| | musculus] | | | | |
| gi\|13431313\|sp\|Q9WU60\| | ATTRACTIN | 1428 | 679/1174 | 871/1174 | 0.0 |
| ATRN_MOUSE | PRECURSOR | | (57%) | (73%) | |
| | (MAHOGANY | | | | |
| | PROTEIN) | | | | |
| gi\|6912258\|ref\|NP_036202.1\| | attractin; | 1198 | 703/1210 | 894/1210 | 0.0 |
| | attractin | | (58%) | (73%) | |
| | (with | | | | |
| | dipeptidylpeptidase | | | | |
| | IV | | | | |
| | activity); | | | | |
| | mahogany | | | | |
| | protein | | | | |
| | [Homo | | | | |
| | sapiens] | | | | |
| gi\|12275312\|dbj\|BAB21018.1\| | attractin | 1275 | 680/1177 | 870/1177 | 0.0 |
| | [Rattus | | (57%) | (73%) | |
| | norvegicus] | | | | |

The homology of these sequences is shown graphically in the ClustalW analysis shown in Table 2D.

[image]

[image]

[image]

[image]

Tables 2E-I list the domain description from DOMAIN analysis results against NOV2. This indicates that the NOV2 sequence has properties similar to those of other proteins known to contain this domain.

TABLE 2E

Domain Analysis of NOV2
gnl|Smart|smart00034, CLECT, C-type lectin (CTL) or carbohydrate-
recognition domain (CRD); Many of these domains function as calcium-
dependent carbohydrate binding modules. (SEQ ID NO: 67)
CD-Length = 124 residues, 100.0% aligned
Score = 70.1 bits (170), Expect = 7e−13

| | | | |
|---|---|---|---|
| Query: | 708 | CGEGW-SHIGDACLRVNSSRENYDNAKLYCYNLSGNLASLTTSKEVEFVLDEIQKYTQQK | 766 |
| | | \| \|\| \|+ \| \| + ++ ++ + +\|+ +\| +\| +\|\|\|+ + +\| +\|+\| ++ | |
| Sbjct: | 1 | CPSGWVSYPGGKCYKFSTEKKTWADAQAFCQSLGAHLASIHSEEENDFLLSLLKNSNSDY | 60 |
| Query: | 767 | VSPWVGLRKINI-SYWGWEDMSPFTNTTLQWLPGEPNDSGFCAYLERAAVAGLKANPCTS | 825 |
| | | \|+\|\| + + \| \| \| \| + + \| \|\|\|\| \|\| \| \| + \|\|\| | |
| Sbjct: | 61 | Y--WIGLSRPDSNGSWQWSDGSGPVDYS-NWAPGEPGGSGNCVVLSTSGGGKWNDVSCTS | 117 |
| Query: | 826 | MANGLVCE | 833 |
| | | +\|\| | |
| Sbjct: | 118 | -KLPFICE | 124 |

TABLE 2F

Domain Analysis of NOV2
gnl|Smart|smart00042, CUB, Domain first found in Clr, Cls, uEGF, and
bone morphogenetic protein.; This domain is found mostly among
developmentally-regulated proteins. Spermadhesins contain only this
domain.. (SEQ ID NO: 68)
CD-Length = 114 residues, 96.5% aligned
Score = 66.2 bits (160), Expect = 1e−11

| | | | |
|---|---|---|---|
| Query: | 58 | RLTEPSGYLT--DGPINYKYKTKCTWLIEGPNA-VLRLRFNHFATE----CSWDHMYVYD | 110 |
| | | \|\| \|\| +\| + \| +\| \| \| \| \| + \|+\| \| \| \|++\|++ +\|\| | |
| Sbjct: | 4 | TLTASSGTITSPNYPNSYPNNLNCVWTISAPPGYRIELKFTDFDLESSDNCTYDYVEIYD | 63 |
| Query: | 111 | GDSIYAPLIASFSGLIVPEIRGNETVPEVVTTSGYALLHFFSDAAYNLTGFNIFYSI | 167 |
| | | \| \| +\|\|+ \| \| +\| \| + ++\| + \| \|\|++ \|\|+ \|\| | |
| Sbjct: | 64 | GPSTSSPLLGRPCGSELPP-------PIISSSSNSMTVTFVSDSSVQKRGFSARYSA | 113 |

TABLE 2G

Domain Analysis of NOV2
gnl|Smart|smart00042, CUB, Domain first found in Clr, Cls, uEGF, and
bone morphogenetic protein.; This domain is found mostly among
developmentally-regulated proteins. Spermadhesins contain only this
domain.. (SEQ ID NO: 68)
CD-Length = 114 residues, 96.5% aligned
Score = 66.2 bits (160). Expect = 1e−11

| | | | |
|---|---|---|---|
| Query: | 58 | RLTEPSGYLT--DGPINYICYKTKCTWLIEGPNA-VLRLRFNHFATE---CSWDHMYVYD | 110 |
| | | \|\| \|\| +\| + \| +\| \| \| \| \| + \|+\| \| \| \|++\|++ +\|\| | |
| Sbjct: | 4 | TLTASSGTITSPNYPNSYPNNLNCVWTISAPPGYRIELKFTDFDLESSDNCTYDYVEIYD | 63 |
| Query: | 111 | GDSIYAPLIASFSGLIVPEIRGNETVPEVVTTSGYALLHFFSDAAYNLTGFNIFYSI | 167 |
| | | \| \| +\|\|+ \| \| +\| \| + ++\| + \| \|\|++ \|\|+ \|\| | |
| Sbjct: | 64 | GDSTSSPLLGRFCGSELPP-------PIISSSSNSMTVTFVSDSSVQKRGFSARYSA | 113 |

TABLE 2H

Domain Analysis of NOV2
gnl/Pfam/pfam00431, CUB, CUB domain. (SEQ ID NO: 69)
CD-Length = 110 residues, 97.3% aligned
Score = 63.9 bits (154), Expect = 5e−11

| | | | |
|---|---|---|---|
| Query: | 58 | RLTEPSGYLT--DGPINYKYKTKCTWLIEGPNAV-LRLRFNHFATE----CSWDHWIVYD | 110 |
| | | \|\|\| \|\| ++ + \| +\| +\| \| \| \| + \| \| \| \| \| +\|++ + \| | |
| Sbjct: | 4 | VLTESSGSISSPNYPNDYPPKNECVWTIRAPPGYRVELTFQDFDLEDHTGCRYDYVEIRD | 63 |
| Query: | 111 | GDSIYAPLIASFSGLIVPEIRGNETVPEVVTTSGYALLHFFSDAAYNLTGFNIFY | 165 |
| | | \|\| +\|\|+ \| \| \| ++\|++\| + \| \|\|\|+ + \|\| \| | |
| Sbjct: | 64 | GDGSSSPLLGKFCGSGPP--------EDIVSSSNRMTIKFVSDASVSKRGFKATY | 110 |

TABLE 2I

Domain Analysis of NOV2
gnl/Pfam/pfam00059, lectin_c, Lectin C-type domain. This family
includes both long and short form C-type (SEQ ID NO: 70)
CD-Length = 107 residues, 100.0% aligned
Score = 49.3 bits (116), Expect = 1e−06

| | | | |
|---|---|---|---|
| Query: | 725 | SRENYDNAKLYCYNLSGNLASLTTSKEVEFVLDEIQKYTQQKVSPWVGLRKINIS-YWGW | 783 |
| | | + + \|+ \| \| \| \| \|+ +++\| +\|+ + \|+\|\| \|\| \| \| | |
| Sbjct: | 1 | ESKTWAEAQAACQKLGGGLVSIQSAEEQDFLTSLTKAS---NSYAWIGLTDINTEGTWVW | 57 |
| Query: | 784 | EDMSPFTNTTLQWLPGEPND---SGFCAYLERAAVAGLKANPCTSMANGLVCEK | 834 |
| | | \| \|\| \| \| \|\|\|\|\|+ \| + \|\| \| \|\|\| | |
| Sbjct: | 58 | TDGSPVNYTN--WAPGEPNNRGNKEDCVEIYTDG-NKWNDEPCGSK-LPYVCEF | 107 |

The protein of invention is highly homologous to the protein attractin, which is a membrane-associated or secreted molecule (depending upon the splice variant) in activated T cells. It has a protease activity and is thought to modify the N-terminals of cytokines and chemokines, enabling the cells to interact and form clusters. The mouse ortholog of soluble attractin has been demonstrated to play a role in obesity and metabolic regulation. The protein of invention shows characteristic domains involved in protein-protein interactions, such as the CUB domain and the kelch motif. It also shows the presence of 4 plexin repeats, a lectin C-type domain and two laminin EGF-like domains, thus sharing its domain structure with attractin. Although attractin is predicted to be localized in the mitochondrial matrix, like the protein of invention, it is either secreted or located as a transmembrane protein at the plasma membrane. The protein of invention may therefore have diverse physiological roles in the tissues that it is expressed.

Attractin is a rapidly upregulated membrane-associated molecule on activated T cells (Duke-Cohan J S, et al. Adv Exp Med Biol 2000;477:173-85). It is a member of the CUB family of extracellular guidance and development proteins, sharing with them a protease activity similar to that of Dipeptidyl peptidase IV (DPPIV/CD26). Most remarkably, and in sharp contrast to CD26, it is released from the T cell and is presumed to be a major source of a soluble serum-circulating attractin. Genomic sequencing reveals that the soluble form is not a proteolytic product of the membrane form, but is in fact the result of alternative splicing. Recent results prove that the loss of murine membrane attractin results in the mahogany mutation with severe repercussions upon skin pigmentation and control of energy metabolism. In each of these latter instances, there is a strong likelihood that attractin is moderating the interaction of cytokines with their respective receptors. Attractin is likely performing a similar function in the immune system through capture and proteolytic modification of the N-terminals of several cytokines and chemokines. This regulatory activity allows cells to interact and form immunoregulatory clusters and subsequently aids in downregulating chemokine/cytokine activity once a response has been initiated. These two properties are likely to be affected by the balance of membrane-expressed to soluble attractin.

Attractin was initially identified as a soluble human plasma protein with dipeptidyl peptidase IV activity that is expressed and released by activated T lymphocytes. It has also been identified as the product of the murine mahogany gene with connections to control of pigmentation and energy metabolism. (Tang W et al., Proc Natl Acad Sci USA 2000 May 23;97(11):6025-30). The mahogany product, however, is a transmembrane protein, raising the possibility of a human membrane attractin in addition to the secreted form. The genomic structure of human attractin reveals that soluble attractin arises from transcription of 25 sequential exons on human chromosome 20p13, where the 3′ terminal exon contains sequence from a long interspersed nuclear element-1 (LINE-1) retrotransposon element that includes a stop codon and a polyadenylation signal. The mRNA isoform for membrane attractin splices over the LINE-1 exon and includes five exons encoding transmembrane and cytoplasmic domains with organization and coding potential almost identical to that of the mouse gene. The relative abundance of soluble and transmembrane isoforns measured by reverse transcription-PCR is differentially regulated in lymphoid tissues. Because activation of peripheral blood leukocytes with phytohemagglutinin induces strong expression of cell surface attractin followed by release of soluble attractin, these results suggest that a genomic event unique to mammals, LINE-1 insertion, has provided an evolutionary mechanism for regulating cell interactions during an inflammatory reaction.

The Mahogany/Attractin gene (Atrn) has been proposed as a downstream mediator of Agouti signaling because yellow hair color and obesity in lethal yellow (A(y)) mice are suppressed by the mahogany (Atrn(mg)) mutation. (Lu Xy et al., FEBS Lett 1999 Nov. 26;462(1-2):101-7). The present study examined the distribution of Atrn mRNA in the brain and spinal cord by in situ hybridization. Atrn mRNA was found widely distributed throughout the central nervous system, with high levels in regions of the olfactory system, some limbic structures, regions of the brainstem, cerebellum and spinal cord. In the hypothalamus, Atrn mRNA was found in specific nuclei including the suprachiasmatic nucleus, the supraoptic nucleus, the medial preoptic nucleus, the paraventricular hypothalamic nucleus, the ventromedial hypothalamic nucleus, and the arcuate nucleus. These results suggest a broad spectrum of physiological functions for the Atrn gene product.

Completely different lines of experimentation have identified attractin, a protein that seems to have multiple roles in regulating physiological processes. (Jackson I J Trends Genet November 1999;15(11):429-31). It affects the balance between agonist and antagonist at receptors on melanocytes, modifies behaviour and basal metabolic rate, and mediates an interaction between activated T cells and macrophages. It may well be a target for development of drugs to treat obesity.

Agouti protein and agouti-related protein are homologous paracrine signalling molecules that normally regulate hair colour and body weight, respectively, by antagonizing signalling through melanocortin receptors. (Gunn T M, et al., Nature 1999 Mar. 1 1;398(6723):152-6). Expression of Agouti is normally limited to the skin, but rare alleles from which Agouti is expressed ubiquitously, such as lethal yellow, have pleiotropic effects that include a yellow coat, obesity, increased linear growth, and immune defects. The mahogany (mg) mutation suppresses the effects of lethal yellow on pigmentation and body weight, and results of our previous genetic studies place mg downstream of transcription of Agouti but upstream of melanocortin receptors. Here positional cloning was used to identify a candidate gene for mahogany, Mgca. The predicted protein encoded by Mgca is a 1,428-amino-acid, single-transmembrane-domain protein that is expressed in many tissues, including pigment cells and the hypothalamus. The extracellular domain of the Mgca protein is the orthologue of human attractin, a circulating molecule produced by activated T cells that has been implicated in immune-cell interactions. These observations provide new insight into the regulation of energy metabolism and indicate a molecular basis for crosstalk between melanocortin-receptor signalling and immune function.

Attractin is a normal human serum glycoprotein of 175 kDa that is rapidly expressed on activated T cells and released extracellularly after 48-72 hr. (Duke-Cohan J S et al., Proc Natl Acad Sci USA 1998 Sep. 15;95(19):11336-41). Attractin has been cloned and, in its natural serum form, it mediates the spreading of monocytes that become the focus for the clustering of nonproliferating T lymphocytes. There are two mRNA species with hematopoietic tissue-specific expression that code for a 134-kDa protein with a putative serine protease catalytic serine, four EGF-like motifs, a CUB domain, a C type lectin domain, and a domain homologous with the ligand-binding region of the common gamma cytokine chain. Except for the latter two domains, the overall structure shares high homology with the Caenorhabditis elegans F33C8.1 protein, suggesting that attractin has evolved new domains and functions in parallel with the development of cell-mediated immunity.

The disclosed NOV2 nucleic acid of the invention encoding a Attractin-like protein includes the nucleic acid whose sequence is provided in Table 2A or a fragment thereof. The invention also includes a mutant or variant nucleic acid any of whose bases may be changed from the corresponding base shown in Table 2A while still encoding a protein that maintains its Attractin-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 29% percent of the bases may be so changed.

The disclosed NOV2 protein of the invention includes the Attractin-like protein whose sequence is provided in Table 2B. The invention also includes a mutant or variant protein any of whose residues may be changed from the corresponding residue shown in Table 2B while still encoding a protein that maintains its Attractin-like activities and physiological functions, or a functional fragment thereof. In the mutant or variant protein, up to about 43% percent of the residues may be so changed.

The NOV2 nucleic acids and proteins of the invention are useful in potential therapeutic applications implicated in Von Hippel-Lindau (VHL) syndrome, Alzheimer's disease, Stroke, Tuberous sclerosis, hypercalceimia, Parkinson's disease, Huntington's disease, Cerebral palsy, Epilepsy, Multiple sclerosis, Ataxia-telangiectasia, Leukodystrophies, Behavioral disorders, Addiction, Anxiety, Pain, Neurodegeneration, Diabetes, Autoimmune disease, Renal artery stenosis, Interstitial nephritis, Glomerulonephritis, Polycystic kidney disease, Systemic lupus erythematosus, Renal tubular acidosis, IgA nephropathy, Hypercalceimia, Diabetes, Pancreatitis, Obesity, Endometriosis, Infertility, Hirschsprung's disease, Crohn's Disease, Appendicitis, Muscular dystrophy, Lesch-Nyhan syndrome, Myasthenia gravis, Cirrhosis, Liver failure, Breast cancer, Ovarian cancer, Prostate cancer, Uterine cancer and/or other pathologies/disorders. The NOV2 nucleic acid encoding Attractin-like protein, and the Attractin-like protein of the invention, or fragments thereof, may further be useful in diagnostic applications, wherein the presence or amount of the nucleic acid or the protein are to be assessed.

NOV2 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV2 protein has multiple hydrophilic regions, each of which can be used as an immunogen. In one embodiment, a contemplated NOV2 epitope is from about amino acids 1 to 20. In another embodiment, a NOV2 epitope is from about amino acids 50 to 130. In additional embodiments, NOV2 epitopes are from about amino acids 140 to 150, from about 180 to 380, from about amino acids 400 to 500, from about amino acids 530 to 550, from about amino acids 580 to 680, from about amino acids 700 to 740, from about amino acids 760 to 780, from about amino acids 820 to 900, and from about amino acids 950 to 1200. These novel proteins can be used in assay systems for functional analysis of various human disorders, which are useful in understanding of pathology of the disease and development of new drug targets for various disorders.

NOV3

NOV3 includes three novel Insulin Like Growth Factor Binding Protein Complex-Acid Labile Subunit (IGFBP-ALS)-like proteins disclosed below. The disclosed sequences have been named NOV3a and NOV3b.

NOV3a

A disclosed NOV3a nucleic acid of 6201 nucleotides (also referred to as 124217931_EXT) encoding a novel Kinase-like protein is shown in Table 3a. An open reading frame was identified beginning with a ATG initiation codon at nucleotides 57-59 and ending with a TGA codon at nucleotides 6199-6201. The start and stop codons are in bold letters.

TABLE 3A
NOV3a Nucleotide Sequence (SEQ ID NO: 8)

ATGTTGAAGTTCAAATATGGAGCGCGGAATCCTTTGGATGCTGGTGCTGCTGAACCCATTGCCAGCCGGGC
CTCCAGGCTGAATCTGTTCTTCCAGGGGAAACCACCCTTTATGACTCAACAGCAGATGTCTCCTCTTTCCC
GAGAAGGGATATTAGATGCCCTCTTTGTTCTCTTTGAAGAATGCAGTCAGCCTGCTCTGATGAAGATTAAG
CACGTGAGCAACTTTGTCCGGAAGTGTTCCGACACCATAGCTGAGTTACAGGAGCTCCAGCCTTCGGCAAA
GGACTTCGAAGTCAGAAGTCTTGTAGGTTGTGGTCACTTTGCTGAAGTGCAGGTGGTAAGAGAGAAAGCAA
CCGGGGACATCTATGCTATGAAGTGATGAAGAAGAAGGCTTTATTGGCCCAGGAGCAGGTTTTCATTTTTT
GAGGAAGAGCGGAACATATTATCTCGAAGCACAAGCCCGTGGATCCCCCAATTACAGTATGCCTTTCAGGA
CAAAAATCACCTTTATCTGGTGATGGAATATCAGCCTGGAGGGGACTTGCTGTCACTTTTGAATAGATATG
AGGACCAGTTAGATGAAAACCTGATACAGTTTTACCTAGCTGAGCTGATTTTGGCTGTTCACAGCGTTCAT
CTGATGGGATACGTGCATCGGGACATCAAGCCTGAGAACATTCTCGTTGACCGCACAGGACACATCAAGCT
GGTGGATTTTGGATCTGCCGCGAAAATGAATTCAAACAAGGTGAATGCCAAACTCCCGATTGGGACCCCAG
ATTACATGGCTCCTGAAGTGCTGACTGTGATGAACGGGGATGGAAAAGGCACCTACGGCCTGGACTGTGAC
TGGTGGTCAGTGGGCGTGATTGCCTATGAGATGATTTATGGGAGATCCCCCTTCGCAGAGGGAACCTCTGC
CAGAACCTTCAATAACATTATGAATTTCCAGCGGTTTTTGAAATTTCCAGATGACCCCAAAGTGAGCAGTG
ACTTTCTTGATCTGATTCAAAGCTTGTTGTGCGGCCAGAAAGAGAGACTGAAGTTTGAAGGTCTTTGCTGC
CATCCTTTCTTCTCTAAAATTGACTGGAACAACATTCGTAACGCTCCTCCCCCCTTCGTTCCCACCCTCAA
GTCTGACGATGACACCTCCAATTTTGATGAACCAGAGAAGAATTCGTGGGTTTCATCCTCTCCGTGCCAGC
TGAGCCCCTCAGGCTTCTCGGGTGAAGAACTGCCGTTTGTGGGGTTTTCGTACAGCAAGGCACTGGGGATT
CTTGGTAGATCTGAGTCTGTTGTGTCGGGTCTGGACTCCCCTGCCAAGACTAGCTCCATGGAAAAGAAACT
TCTCATCAAAAGCAAAGAGCTACAAGACTCTCAGGACAAGTGTCACAAGATGGAGCAGGAAATGACCCGGT
TACATCGGAGAGTGTCAGAGGTGGAGGCTGTGCTTAGTCAGAAGGAGGTGGAGCTGAAGGCCTCTGAGACT
CAGAGATCCCTCCTGGAGCAGGACCTTGCTACCTACATCACAGAATGCAGTAGCTTAAAGCGAAGTTTGGA
GCAAGCACGGATGGAGGTGTCCCAGGAGGATGACAAAGCACTGCAGCTTCTCCATGATATCAGAGAGCAGA
GCCGGAAGCTCCAAGAAATCAAAGAGCAGGAGTACCAGGCTCAAGTGGAAGAAATGAGGTTGATGATGAAT
CAGTTGGAAGAGGATCTTGTCTCAGCAAGAAGACGGAGTGATCTCTACGAATCTGAGCTGAGAGAGTCTCG
GCTTGCTGCTGAAGAATTCAAGCGGAAAGCGACAGAATGTCAGCATAAACTGTTGAAGGCTAAGGATCAGG
GGAAGCCTGAAGTGGGAGAATATGCGAAACTGGAGAAGATCAATGCTGAGCAGCAGCTCAAAATTCAGGAG
CTCCAAGAGAAACTGGAGAAGGCTGTAAAAGCCAGCACGGAGGCCACCGAGCTGCTGCAGAATATCCGCCA
GGCAAAGGAGCGAGCCGAGAGGGAGCTGGAGAAGCTGCAGAACCGAGAGGATTCTTCTGAAGGCATCAGAA
AGAAGCTGGTGGAAGCTGAGGAACGCCGCCATTCTCTGGAGAACAAGGTAAAGAGACTAGAGACCATGGAG
CGTAGAGAAAACAGACTGAAGGATGACATCCAGACAAAATCCCAACAGATCCAGCAGATGGCTGATAAAAT
TCTGGAGCTCGAAGAGAAACATCCGGAGGCCCAAGTCTCAGCCCAGCACCTAGAAGTGCACCTGAAACAGA
AAGAGCAGCACTATGAGGAAAAGATTAAAGTATTGGACAATCAGATAAAGAAAGACCTGGCTGACAAGGAG
ACACTGGAGAACATGATGCAGAGACACGAGGAGGAGGCCCATGAGAAGGGCAAAATTCTCAGCGAACAGAA
GGCGATGATCAATGCTATGGATTCCAAGATCAGATCCCTGGAACAGAGGATTGTGGAACTGTCTGAAGCCA
ATAAACTTGCAGCAAATAGCAGTCTTTTTACCCAAAGGAACATGAAGGCCCAAGAAGAGATGATTTCTGAA
CTCAGGCAACAGAAATTTTACCTGGAGACACAGGCTGGGAAGTTGGAGGCCCAGAACCGAAAACTGGAGGA
GCAGCTGGAGAAGATCAGCCACCAAGACCACAGTGACAAGAATCGGCTGCTGGAACTGGAGACAAGATTGC
GGGAGGTGAGTCTAGAGCACGAGGAGCAGAAACTGGAGCTCAAGCGCCAGCTCACAGAGCTACAGCTCTCC
CTGCAGGAGCGCGAGTCACAGTTGACAGCCCTGCAGGCTGCACGGGCGGCCCTGGAGAGCCAGCTTCGCCA
GGCGAAGACAGAGCTGGAAGAGACCACAGCAGAAGCTGAAGAGGAGATCCAGGCACTCACGGCACATAGAG
ATGAAATCCAGCGCAAATTTGATGCTCTTCGTAACAGCTGTACTGTGATCACAGACCTCGAGGAGCAGCTA
AACCAGCTGACCGAGGACAACGCTGAACTCAACAACCAAAACTTCTACTTGTCCAAACAACTCGATGAGGC
TTCTGGCGCCAACGACGAGATTGTACAACTGCGAAGTGAAGTGGACCATCTCCGCCGGGAGATCACGGAAC
GAGAGATGCAGCTTACCAGCCAGAAGCAAACGATGGAGGCTCTGAAGACCACGTGCACCATGCTGGAGGAA
CAGGTCATGGATTTGGAGGCCCTAAACGATGAGCTGCTAGAAAAAGACCGGCAGTGGGAGGCCTGGAGGAG
CGTCCTGGGTGATGAGAAATCCCAGTTTGAGTGTCGGGTTCGAGAGCTGCAGAGGATGCTGGACACCGAGA
AACAGAGCAGGGCGAGAGCCGATCAGCGGATCACCGAGTCTCGCCAGGTGGTGGAGCTGGCAGTGAAGGAG
CACAAGGCTGAGATTCTCGCTCTGCAGCAGGCTCTCAAAGAGCAGAAGCTGAAGGCCGAGAGCCTCTCTGA
CAAGCTCAATGACCTGGAGAAGAAGCATGCTATGCTTGAAATGAATGCCCGAAGCTTACAGCAGAAGCTGG
AGACTGAACGAGAGCTCAAACAGAGGCTTCTGGAAGAGCAAGCCAAATTACAGCAGCAGATGGACCTGCAG
AAAAATCACATTTTCCGTCTGACTCAAGGACTGCAAGAAGCTCTAGATCGGGCTGATCTACTGAAGACAGA
AAGAAGTGACTTGGAGTATCAGCTGGAAAACATTCAGGTGCTCTATTCTCATGAAAAGGTGAAAATGGAAG
GCACTATTTCTCAACAAACCAAACTCATTGATTTTCTGCAAGCCAAAATGGACCAACCTGCTAAAAAGAAA
AAGGTGCCTCTGCAGTACAATGAGCTGAAGCTGGCCCTGGAGAAGGAGAAAGCTCGCTGTGCAGAGCTAGA
GGAAGCCCTTCAGAAGACCCGCATCGAGCTCCGGTCCGCCCGGGAGGAAGCTGCCCACCGCAAAGCAACGG
ACCACCCACACCCATCCACGCCAGCCACCGCGAGGCAGCAGATCGCCATGTCTGCCATCGTGCGGTCGCCA
GAGCACCAGCCCAGTGCCATGAGCCTGCTGGCCCCGCCATCCAGCCGCAGAAAGGAGTCTTCAACTCCAGA
GGAATTTAGTCGGCGTCTTAAGGAACGCATGCACCACAATATTCCTCACCGATTCAACGTAGGACTGAACA
TGCGAGCCACAAAGTGTGCTGTGTGTCTGGATACCGTGCACTTTGGACGCCAGGCATCCAAATGTCTAGAA
TGTCAGGTGATGTGTCACCCCAAGTGCTCCACGTGCTTGCCAGCCACCTGCGGCTTGCCTGCTGAATATGC
CACACACTTCACCGAGGCCTTCTGCCGTGACAAAATGAACTCCCCAGGTCTCCAGACCAAGGAGCCCAGCA
GCAGCTTGCACCTGGAAGGGTGGATGAAGGTGCCCAGGAATAACAAACGAGGACAGCAAGGCTGGGACAGG
AAGTACATTGTCCTGGAGGGATCAAAAGTCCTCATTTATGACAATGAAGCCAGAGAAGCTGGACAGAGGCC
GGTGGAAGAATTTGAGCTGTGCCTTCCCGACGGGGATGTATCTATTCATGGTGCCGTTGGTGCTTCCGAAC
TCGCAAATACAGCCAAAGCAGATGTCCCATACATACTGAAGATGGAATCTCACCCGCACACCACCTGCTGG
CCCGGGAGAACCCTCTACTTGCTAGCTCCCAGCTTCCCTGACAAACAGCGCTGGGTCACCGCCTTAGAATC
AGTTGTCGCAGGTGGGAGAGTTTCTAGGGAAAAAGCAGAAGCTGATGCTAAACTGCTTGGAAACTCCCTGC
TGAAACTGGAAGGTGATGACCGTCTAGACATGAACTGCACGCTGCCCTTCAGTGACCAGGTAGTGTTGGTG
GGCACCGAGGAAGGGCTCTACGCCCTGAATGTCTTGAAAAACTCCCTAACCCATGTCCCAGGAATTGGAGC
AGTCTTCCAAATTTATATTATCAAGGACCTGGAGAAGCTACTCATGATAGCAGGTGAAGAGCGGGCACTGT
GTCTTGTGGACGTGAAGAAAGTGAAACAGTCCCTGGCCCAGTCCCACCTGCCTGCCCAGCCCGACATCTCA
CCCAACATTTTTGAAGCTGTCAAGGGCTGCCACTTGTTTGGGGCAGGCAAGATTGAGAACGGGCTCTGCAT
CTGTGCAGCCATGCCCAGCAAAGTCGTCATTCTCCGCTACAACGAAAACCTCAGCAAATACTGCATCCGGA
AAGAGATAGAGACCTCAGAGCCCTGCAGCTGTATCCACTTCACCATTACAGTATCCTCATTGGAACCAAAT
AAATTCTACGAAATCGACATGAAGCAGTACACGCTCGAGGAATTCCTGGATAAGAATGACCATTCCTTGGC
ACCTGCTGTGTTTGCCGCCTCTTCCAACAGCTTCCCTGTCTCAATCGTGCAGGTGAACAGCGCAGGGCAGC
GAGAGGAGTACTTGCTGTGTTTCCACGAATTTGGAGTGTTCGTGGATTCTTACGGAAGACGTAGCCGCACA
GACGATCTCAAGTGGAGTCGCTTACCTTTGGCCTTTGCCTACAGAGAACCCTATCTGTTTGTGACCCACTT
CAACTCACTCGAAGTAATTGAGATCCAGGCACGCTCCTCAGCAGGGACCCCTGCCCGAGCGTACCTGGACA
TCCCGAACCCGCGCTACCTGGGCCCTGCCATTTCCTCAGGAGCGATTTACTTGGCGTCCTCATACCAGGAT
AAATTAAGGGTCATTTGCTGCAAGGGAAACCTCGTGAAGGAGTCCGGCACTGAACACCACCGGGGCCCGTC
CACCTCCCGCAGCAGCCCCAACAAGCGAGGCCCACCCACGTACAACGAGCACATCACCAAGCGCGTGGCCT
CCAGCCCAGCGCCGCCCGAAGGCCCCAGCCACCCGCGAGAGCCAAGCACACCCCACCGCTACCGCGAGGGG
CGGACCGAGCTGCGCAGGGACAACTCTCCTGGCCGCCCCCTGGAGCGAGAGAAGTCCCCCGGCCGGATGCT
CAGCACGCGGAGAGAGCGGTCCCCCGGGAGGCTGTTTGAAGACAGCAGCAGGGGCCGGCTGCCTGCGGGAG
CCGTGAGGACCCCGCTGTCCCAGGTGAACAAGGTGAGGCAGCATTCCGAGGCCTGTGTGTCTGTTGCGGAG
GCCAGGAGTGACTTGGGGAACTGA

The disclosed NOV3a nucleic acid sequence maps to chromosome 13 and has 5518 of 6158 bases (89%) identical to rho/rac-interacting citron kinase (Crik) mRNA from Mus musculus (GENBANK-ID:AF086824) (E=0.0).

A disclosed NOV3a protein (SEQ ID NO:9) encoded by SEQ ID NO:8 has 2066 amino acid residues, and is presented using the one-letter code in Table 3B. Signal P, Psort and/or Hydropathy results predict that NOV3a does not have a signal peptide, and is likely to be localized to the nucleus with a certainty of 0.9800. In other embodiments NOV3a is also likely to be localized to microbody (peroxisome) with a certainty of 0.3000, to the mitochondrial membrane space with a certainty of 0.1000, or to the lysosome (lumen) with a certainty of 0.1000.

TABLE 3B
Encoded NOV3a protein sequence (SEQ ID NO: 9).

MLKFKYGARNPLDAGAAEPIASRASRLNLFFQGKPPFMTQQQMSPLSREGILDALFVLFEECSQPALMKIK
HVSNFVRKCSDTIAELQELQPSAKDFEVRSLVGCGHFAEVQVVREKATGDIYAMKVMKKKALLAQEQVSFF
EEERNILSRSTSPWIPQLQYAFQDKNHLYLVMEYQPGGDLLSLLNRYEDQLDENLIQFYLAELILAVHSVH
LMGYVHRDIKPENILVDRTGHIKLVDFGSAAKMNSNKVNAKLPIGTPDYMAPEVLTVMNGDGKGTYGLDCD
WWSVGVIAYEMIYGRSPFAEGTSARTFNNIMNPQRFLKFPDDPKVSSDFLDLIQSLLCCQKERLKFEGLCC
HPFFSKIDWNNIRNAPPPFVPTLKSDDDTSNFDEPEKNSWVSSSPCQLSPSGFSGEELPFVGFSYSKALGI
LGRSESVVSGLDSPAKTSSMEKKLLIKSKELQDSQDKCHKMEQEMTRLHRRVSEVEAVLSQKEVELKASET
QRSLLEQDLATYITECSSLKRSLEQARMEVSQEDDKALQLLHDIREQSRKLQEIKEQEYQAQVEEMRLMMN
QLEEDLVSARRRSDLYESELRESRLAAEEFKRKATECQHKLLKAKDQGKPEVGEYAKLEKINAEQQLKIQE
LQEKLEKAVKASTEATELLQNIRQAKERAERELEKLQNREDSSEGIRKKLVEAEERRHSLENKVKRLETME
RRENRLKDDIQTKSQQIQQMADKILELEEKHREAQVSAQHLEVHLKQKEQHYEEKIKVLDNQIKKDLADKE
TLENMMQRHEEEAHEKGKILSEQKAMINAMDSKIRSLEQRIVELSEANKLAANSSLFTQRNMKAQEEMISE
LRQQKFYLETQAGKLEAQNRKLEEQLEKISHQDESDKNRLLELETRLREVSLEHEEQKLELKRQLTELQLS
LQERESQLTALQAARAALESQLRQAKTELEETTAEAEEEIQALTAHRDEIQRKFDALRNSCTVITDLEEQL
NQLTEDNAELNNQNFYLSKQLDEASGANDEIVQLRSEVDHLRREITEREMQLTSQKQTMEALKTICTMLEE
QVMDLEALNDELLEKERQWEAWRSVLGDEKSQFECRVRELQRNLDTEKQSRARADQRITESRQVVELAVKE
HKAEILALQQALKEQKLKAESLSDKLNDLEKKHAMLEMNARSLQQKLETERELKQRLLEEQAKLQQQMDLQ
KNHIFRLTQGLQEALDRADLLKTERSDLEYQLENIQVLYSHEKVKMEGTISQQTKLIDFLQAKMDQPAKKK
KVPLQYNELKLALEKEKARCAELEEALQKTRIELRSAREEAAHRKATDHPHPSTPATARQQIANSAIVRSP
EHQPSAMSLLAPPSSRRKESSTPEEFSRRLKERMHHNIPHRFNVGLNMRATKCAVCLDTVHFGRQASKCLE
CQVMCHPKCSTCLPATCGLPAEYATNFTEAFCRDKMNSPGLQTKEPSSSLHLEGWMKVPRNNKRGQQGWDR
KYIVLEGSKVLIYDNEAREAGQRPVEEFELCLPDGDVSIHGAVGASELANTAKADVPYILKMESHPHTTCW
PGRTLYLLAPSFPDKQRWVTALESVVAGGRVSREKAEADAKLLGNSLLKLEGDDRLDMNCTLPFSDQVVLV
GTEEGLYALNVLKNSLTHVPGIGAVFQIYIIKDLEKLLMIAGEERALCLVDVKKVKQSLAQSHLPAQPDIS
PNIFEAVKGCHLFGAGKIENGLCICAAMPSKVVILRYNENLSKYCIRKEIETSEPCSCIHFTNYSILIGTN
KFYEIDMKQYTLEEFLDKNDHSLAPAVFAASSNSFPVSIVQVNSAGQREEYLLCFHEFGVFVDSYGRRSRT
DDLKWSRLPLAFAYREPYLFVTHFNSLEVIEIQARSSAGTPARAYLDIPNPRYLGPAISSGAIYLASSYQD
KLRVICCKGNLVKESGTEHHRGPSTSRSSPNKRGPPTYNEHITKRVASSPAPPEGPSMPREPSTPHRYREG
RTELRRDKSPGRPLEREKSPGRMLSTRRERSPGRLFEDSSRGRLPAGAVRTPLSQVNKVRQHSEACVSVAE
ARSDLGN

The disclosed NOV3a amino acid has 1969 of 2053 amino acid residues (95%) identical to, and 2009 of 2053 amino acid residues (97%) similar to, the 2055 amino acid residue rho/rac-interacting citron kinase (Crik) protein from Mus musculus (SPTREMBL-ACC:088938) (E=0.0).

TaqMan expression data for NOV3a is found below is Example 2.

NOV3b

A disclosed NOV3b nucleic acid of 6189 nucleotides (designated CuraGen Acc. No. CG106764-01) encoding a novel RHO/RAC-interacting citron kinase-like is shown in Table 3C. An open reading frame was identified beginning with an ATG initiation codon at nucleotides 1-3 and ending with a TAA codon at nucleotides 6160-6162. A putative untranslated region downstream from the termination codon is underlined in Table 3C, and the start and stop codons are in bold letters.

TABLE 3C
NOV3b Nucleotide Sequence (SEQ ID NO: 10)

ATGTTGAAGTTCAAATATGGAGCGCGGAATCCTTTGGATGCTGGTGCTGCTGAACCCATTGCCAGCCGGGCCTCC
AGGCTGAATCTGTTCTTCCAGGGGAAACCACCCTTTATGACTCAACAGCAGATGTCTCCTCTTTCCCGAGAAGGG
ATATTAGATGCCCTCTTTGTTCTCTTTGAAGAATGCAGTCAGCCTGCTCTGATGAAGATTAAGCACGTGAGCAAC
TTTGTCCGGAAGTGTTCCGACACCATAGCTGAGTTACAGGAGCTCCAGCCTTCGGCAAAGGACTTCGAAGTCAGA
AGTCTTGTAGGTTGTGGTCACTTTGCTGAAGTGCAGGTGGTAAGAGAGAAAGCAACCGGGGACATCTATGCTATG
AAAGTGATGAAGAAGAAGGCTTTATTGGCCCAGGAGCAGGTTTCATTTTTTGAGGAAGAGCGGAACATATTATCT
CGAAGCACAAGCCCGTGGATCCCCCAATTACAGTATGCCTTTCAGGACAAAAATCACCTTTATCTGGTGATGGAA
TATCAGCCTGGAGGGGACTTGCTGTCACTTTTGAATAGATATGAGGACCAGTTAGATGAAAACCTGATACAGTTT
TACCTAGCTGAGCTGATTTTGGCTGTTCACACCGTTCATCTGATGGGATACGTGCATCGGGACATCAAGCCTGAG
AACATTCTCGTTGACCGCACAGGACACATCAAGCTGGTGGATTTTGGATCTGCCGCGAAAATGAATTCAAACAAG
GTGAATGCCAAACTCCCGATTGGGACCCCAGATTACATGGCTCCTCAAGTGCTGACTGTGATGAACGGGGATGCA
AAAGGCACCTACGGCCTGGACTGTGACTGGTGGTCAGTGGGCGTGATTGCCTATCAGATGATTTATGGGAGATCC
CCCTTCGCAGAGGGAACCTCTGCCAGAACCTTCAATAACATTATGAATTTCCAGCGGTTTTTGAAATTTCCAGAT
GACCCCAAAGTGAGCAGTGACTTTCTTGATCTGATTCAAAGCTTGTTGTGCGGCCAGAAAGAGAGACTGAAGTTT
GAAGGTCTTTGCTGCCATCCTTTCTTCTCTAAAATTGACTGGAACAACATTCGTAACGCTCCTCCCCCCTTCGTT
CCCACCCTCAAGTCTGACGATGACACCTCCAATTTTGATGAACCAGAGAAGAATTCGTGGGTTTCATCCTCTCCG
TGCCAGCTGAGCCCCTCAGGCTTCTCGGGTGAAGAACTGCCGTTTGTGGGGTTTTCGTACAGCAAGGCACTGGGG
ATTCTTGGTAGATCTGAGTCTGTTGTGTCGGGTCTGGACTCCCCTGCCAAGACTAGCTCCATGGAAAAGAAACTT
CTCATCAAAAGCAAAGAGCTACAAGACTCTCAGGACAAGTGTCACAAGATGGAGCAGGAAATGACCCGGTTACAT
CGGAGAGTGTCAGAGGTGGAGGCTGTGCTTAGTCAGAAGGAGGTGGAGCTGAAGGCCTCTGAGACTCAGAGATCC
CTCCTGGAGCAGGACCTTGCTACCTACATCACAGAATGCAGTAGCTTAAAGCGAAGTTTGGAGCAAGCACGGATG
GAGGTGTCCCAGGAGGATGACAAAGCACTGCAGCTTCTCCATGATATCAGAGAGCAGAGCCGGAAGCTCCAAGAA
ATCAAAGAGCAGGAGTACCAGGCTCAAGTGGAAGAAATGAGGTTGATGATGAATCAGTTGGAAGAGGATCTTGTC
TCAGCAAGAAGACGGAGTGATCTCTACGAATCTGAGCTGAGAGAGTCTCGGCTTGCTGCTGAAGAATTCAAGCGG
AAAGCGACAGAATGTCAGCATAAACTGTTGAAGGCTAAGGATCAGGGGAAGCCTGAAGTGGGAGAATATGCGAAA
CTGGAGAAGATCAATGCTGAGCAGCAGCTCAAAATTCAGGAGCTCCAAGAGAAACTGGAGAAGGCTGTAAAAGCC
AGCACGGAGGCCACCGAGCTGCTGCAGAATATCCGCCAGGCAAAGGAGCGAGCCGAGAGGGAGCTGGAGAAGCTG
CAGAACCGAGAGGATTCTTCTGAAGGCATCAGAAAGAAGCTGGTGGAAGCTGAGGAACGCCGCCATTCTCTGGAG
AACAAGGTAAAGAGACTAGAGACCATGGAGCGTAGAGAAAACAGACTGAAGGATGACATCCAGACAAAATCCCAA
CAGATCCAGCAGATGGCTGATAAAATTCTGGAGCTCGAAGAGAAACATCGGGAGGCCCAAGTCTCAGCCCAGCAC
CTAGAAGTGCACCTGAAACAGAAAGAGCAGCACTATGAGGAAAAGATTAAAGTATTGGACAATCAGATAAAGAAA
GACCTGGCTGACAAGGAGACACTGGAGAACATGATGCAGAGACACGAGGAGGAGGCCCATGAGAAGGGCAAAATT
CTCAGCGAACAGAAGGCGATGATCAATGCTATGGATTCCAAGATCAGATCCCTGGAACAGAGGATTGTGGAACTG
TCTGAAGCCAATAAACTTGCAGCAAATAGCAGTCTTTTTACCCAAAGGAACATGAAGGCCCAAGAAGAGATGATT
TCTGAACTCAGGCAACAGAAATTTTACCTGGAGACACAGGCTGGGAAGTTGGAGGCCCAGAACCGAAAACTGGAG
GAGCAGCTGGAGAAGATCAGCCACCAAGACCACAGTGACAAGAATCGGCTGCTGGAACTCGAGACAAGATTGCGG
GAGGTGACTCTAGAGCACGAGGAGCAGAAACTGGAGCTCAAGCGCCAGCTCACAGAGCTACAGCTCTCCCTGCAG
GAGCGCGAGTCACAGTTGACAGCCCTGCACGCTGCACGGGCGGCCCTGGAGAGCCAGCTTCGCCAGGCGAAGACA
GAGCTGGAAGAGACCACAGCAGAAGCTGAAGAGGAGATCCAGGCACTCACGGCACATAGAGATGAAATCCAGCGC
AAATTTGATGCTCTTCGTAACAGCTGTACTGTGATCACAGACCTGGACGAGCAGCTAAACCAGCTGACCGAGGAC
AACGCTGAACTCAACAACCAAAACTTCTACTTGTCCAAACAACTCQATGAGGCTTCTGGCGCCAACGACGAGATT
GTACAACTGCGAAGTGAAGTGGACCATCTCCGCCGGGAGATCACGGAACGAGAGATGCAGCTTACCAGCCAGAAG
CAAACGATGGAGGCTCTGAAGACCACGTGCACCATGCTGGAGGAACAGGTCATGGATTTGGAGGCCCTAAACGAT
GAGCTGCTAGAAAAAGAGCGGCAGTGGGAGGCCTGGAGGAGCGTCCTGGGTGATGACAAATCCCAGTTTGAGTGT
CGGGTTCGAGAGCTGCAGAGGATGCTGGACACCGAGAAACAGAGCAGGGCGAGAGCCGATCAGCGGATCACCGAG
TCTCGCCAGGTGGTGGAGCTGGCAGTGAAGGAGCACAAGGCTGAGATTCTCGCTCTGCAGCAGGCTCTCAAAGAG
CAGAAGCTGAAGGCCGAGAGCCTCTCTGACAAGCTCAATGACCTGGAGAAGAAGCATGCTATGCTTGAAATGAAT
GCCCGAAGCTTACAGCAGAAGCTGGAGACTGAACGAGAGCTCAAACAGAGGCTTCIGGAAGAGCAAGCCAAATTA
CAGCAGCAGATGGACCTGCAGAAAAATCACATTTTCCGTCTGACTCAAGGACTGCAAGAAGCTCTAGATCGGGCT
GATCTACTGAAGACAGAAAGAAGTGACTTGGAGTATCAGCTGGAAAACATTCAGGTGCTCTATTCTCATGAAAAG
GTGAAAATGGAAGGCACTATTTCTCAACAAACCAAACTCATTGATTTTCTGCAAGCCAAAATGGACCAACCTGCT
AAAAAGAAAAAGGTGCCTCTGCAGTACAATGAGCTGAAGCTGGCCCTGGAGAAGGAGAAAGCTCCCTGTGCAGAG
CTAGAGGAAGCCCTTCAGAAGACCCGCATCGAGCTCCGGTCCGCCCGGGAGGAAGCTGCCCACCGCAAAGCAACG
GACCACCCACACCCATCCACGCCAGCCACCGCGAGGCAGCAGATCGCCATGTCTGCCATCGTCCGGTCGCCAGAG
CACCAGCCCAGTGCCATGAGCCTGCTGGCCCCGCCATCCAGCCGCAGAAAGGAGTCTTCAACTCCAGAGGAATTT
AGTCGGCGTCTTAAGGAACGCATGCACCACAATATTCCTCACCGATTCAACGTAGGACTGAACATGCGAGCCACA
AAGTGTGCTGTGTGTCTGGATACCGTGCACTTTGGACGCCAGGCATCCAAATGTCTAGAATGTCAGGTGATGTGT
CACCCCAAGTGCTCCACGTGCTTGCCAGCCACCTGCGGCTTGCCTGCTGAATATGCCACACACTTCACCGAGGCC
TTCTGCCGTGACAAAATGAACTCCCCAGGTCTCCAGACCAAGGAGCCCAGCAGCAGCTTGCACCTGGAAGGGTGG
ATGAAGGTGCCCAGGAATAACAAACGAGGACAGCAAGGCTGGGACAGGAAGTACATTGTCCTGGAGGGATCAAAA
GTCCTCATTTATGACAATGAAGCCAGAGAAGCTGGACAGAGGCCGGTGGAAGAATTTGAGCTGTGCCTTCCCGAC
GGGGATGTATCTATTCATGGTGCCGTTGGTGCTTCCGAACTCGCAAATACAGCCAAAGCAGATGTCCCATACATA
CTGAAGATGGAATCTCACCCGCACACCACCTCCTGGCCCGGGAGAACCCTCTACTTGCTAGCTCCCAGCTTCCCT
GACAAACAGCGCTGGGTCACCGCCTTAGAATCAGTTGTCGCAGGTGGGAGACTTTCTAGGGAAAAAGCAGAAGCT
GATGCTAAACTGCTTGGAAACTCCCTGCTGAAACTGGAACGTGATGACCGTCTAGACATGAACTGCACGCTGCCC
TTCAGTGACCAGGTAGTGTTGGTGGGCACCGAGGAAGGGCTCTACGCCCTGAATGTCTTGAAAAACTCCCTAACC
CATGTCCCAGGAATTGGAGCAGTCTTCCAAATTTATATTATCAAGGACCTGGAGAAGCTACTCATGATAGCAGGT
GAAGAGCGGGCACTGTGTCTTGTGGACGTGAAGAAACTGAAACAGTCCCTGGCCCAGTCCCACCTGCCTGCCCAG
CCCGACATCTCACCCAACATTTTTGAAGCTGTCAAGGGCTGCCACTTGTTTGGGGCAGGCAAGATTGAGAACGGG
CTCTGCATCTGTGCAGCCATGCCCAGCAAAGTCGTCATTCTCCGCTACAACGAAAACCTCAGCAAATACTGCATC
CGGAAAGAGATAGAGACCTCAGAGCCCTGCAGCTGTATCCACTTCACCAATTACAGTATCCTCATTGGAACCAAT
AAATTCTACGAAATCGACATGAAGCAGTACACGCTCGAGGAATTCCTGGATAAGAATGACCATTCCTTGGCACCT
GCTGTGTTTGCCGCCTCTTCCAACAGCTTCCCTGTCTCAATCGTGCAGGTGAACAGCGCAGGGCAGCGAGAGGAG
TACTTGCTGTGTTTCCACGAATTTGGAGTGTTCGTGGATTCTTACGGAAGACGTAGCCGCACAGACGATCTCAAG
TGGAGTCGCTTACCTTTGGCCTTTGCCTACAGAGAACCCTATCTGTTTGTGACCCACTTCAACTCACTCGAAGTA
ATTGAGATCCAGGCACGCTCCTCAGCAGGGACCCCTGCCCGAGCGTACCTGGACATCCCGAACCCGCGCTACCTG
GGCCCTGCCATTTCCTCAGGAGCGATTTACTTGGCGTCCTCATACCAGGATAAATTAAGGGTCATTTGCTGCAAG
GGAAACCTCGTGAAGGAGTCCGGCACTGAACACCACCGGGGCCCGTCCACCTCCCGCAGCAGCCCCAACAAGCGA
GGCCCACCCACGTACAACGAGCACATCACCAAGCGCGTGGCCTCCAGCCCAGCGCCGCCCGAAGGCCCCAGCCAC
CCGCGAGAGCCAAGCACACCCCACCGCTACCGCGAGGGGCGGACCGAGCTGCGCAGGGACAAGTCTCCTGGCCGC
CCCCTGGAGCGAGAGAAGTCCCCCGGCCGGATGCTCAGCACGCGGAGAGAGCGGTCCCCCGGGAGGCTGTTTGAA
GACAGCAGCAGGGGCCGGCTGCCTGCGGGAGCCGTGAGGACCCCGCTGTCCCAGGTGAACAAGGTGTGGGACCAG
TCTTCAGTATAAATCTCAGCCAGAAAAACCAACTCCTCA

The disclosed NOV3b nucleic acid sequence of this invention has 2894 of 2908 bases (99%) identity with KIAA1531 mRNA from Homo sapiens (GENBANK-ID: AB040964) (E=0.0).

A NOV3b polypeptide (SEQ ID NO:11) encoded by SEQ ID NO:10 is 2053 amino acid residues and is presented using the one letter code in Table 3D. The SignalP, Psort and Hydropathy, Psort, and/or SignalP data suggest that the NOV3b protein has no signal peptide and may be localized to nucleus with a certainty of 0.9800. In other embodiments, NOV3b may also be localized to the microbody (peroxisome) with a certainty of 0.300, the mitochondrial matrix space with a certainty of 0.100 or the lysosome (lumen) with a certainty of 0.100.

TABLE 3D
Encoded NOV3b protein sequence (SEQ ID NO: 11)

MLKFKYGARNPLDAGAAEPIASRSRLNLFFQGKPPFMTQQQMSPLSREGILDALFLFEECSQPALMKIKHV
SNFVRKCSDTIAELQELQPSAKDFEVRSLVGCGHFAEVQVVREKATGDIYAMKVMKKKALLAQEQVSFFEEER
NILSRSTSPWIPQLQYAFQDKNHLYLVMEYQPGGDLLSLLNRYEDQLDENLIQFYLAELILAVHSVHLMGYVH
RDIKPENILVDRTGHIKLVDFGSAAKMNSNKVNAKLPIGTPDYMAPEVLTVMNGDGKGTYGLDCDWWSVGVIA
YEMIYGRSPFAEGTSARTFNNIMNFQRFLKFPDDPKVSSDFLDLIQSLLCGQKERLKFEGLCCHPFFSKIDWN
NIRNAPPPFVPTLKSDDDTSNFDEPEKNSWVSSSPCQLSPSGFSGEELPFVGFSYSKALGILGRSESVVSGLD
SPAKTSSMEKKLLIKSKELQDSQDKCHKMEQEMTRLHRRVSEVEAVLSQKEVELKASETQRSLLEQDLATYIT
ECSSLKRSLEQARMEVSQEDDKALQLLHDIREQSRKLQEIKEQEYQAQVEEMRLMMNQLEEDLVSARRRSDLY
ESELRESRLAAEEFKRKATECQHKLLKAKDQGKPEVGEYAKLEKINAEQQLKIQELQEKLEKAVKASTEATEL
LQNIRQAKERAERELEKLQNREDSSEGIRKKLVEAEERRHSLENKVKRLETMERRENRLKDDIQTKSQQIQQM
ADKILELEEKHREAQVSAQHLEVHLKQKEQHYEEKIKVLDNQIKKDLADKETLENMMQRHEEEAHEKGKILSE
QKAMINAMDSKIRSLEQRIVELSEANKLAANSSLFTQRNMKAQEEMISELRQQKFYLETQAGKLEAQNRKLEE
QLEKISMQDHSDKNRLLELETRLREVSLEHEEQKLELKRQLTELQLSLQERESQLTALQAARAALESQLRQAK
TELEETTAEAEEEIQALTAHRDEIQRKFDALRNSCTVITDLEEQLNQLTEDNAELNNQNFYLSKQLDEASGAN
DEIVQLRSEVDHLRREITEREMQLTSQKQTMEALKTTCTMLEEQVMDLEALNDELLEKERQWEAWRSVLGDEK
SQFECRVRELQRMLDTEKQSRARADQRITESRQVVELAVKEHKAEILALQQALKEQKLKAESLSDKLNDLEKK
HAMLEMNARSLQQKLETERELKQRLLEEQAKLQQQMDLQKNHIFRLTQGLQEALDRADLLKTERSDLEYQLEN
IQVLYSHEKVKMEGTISQQTKLIDFLQAKMDQPAKKKKVPLQYNELKLALEKEKARCAELEEALQKTRIELRS
AREEAAHRKATDHPHPSTPATARQQIAMSAIVRSPEHQPSAMSLLAPPSSRRKESSTPEEFSRRLKERMHHNI
PHRFNVGLNMRATKCAVCLDTVHFGRQASKCLECQVMCHPKCSTCLPATCGLPAEYATHFTEAFCRDKMNSPG
LQTKEPSSSLHLEGWMKVPRNNKRGQQGWDRKYIVLEGSKVLIYDNEAREAGQRPVEEFELCLPDGDVSIHGA
VGASELANTAKADVPYILKNESHPHTTCWPGRTLYLLAPSFPDKQRWVTALESVVAGGRVSREKAEADAKLLG
NSLLKLEGDDRLDMNCTLPFSDQVVLVGTEEGLYALNVLKNSLTHVPGIGAVFQIYIIKDLEKLLMIAGEERA
LCLVDVKKVKQSLAQSHLPAQPDISPNIFEAVKGCHLFGAGKIENGLCICAAMPSKVVILRYNENLSKYCIRK
EIETSEPCSCIHFTNYSILIGTNKFYEIDMKQYTLEEFLDKNDHSLAPAVFAASSNSFPVSIVQVNSAGQREE
YLLCFHEFGVFVDSYGRRSRTDDLKWSRLPLAFAYREPYLFVTHFNSLEVIEIQARSSAGTPARAYLDIPNPR
YLGPAISSGAIYLASSYQDKLRVICCKGNLVIESGTEHHRGPSTSRSSPNKRGPPTYNEHITKRVASSPAPPE
GPSHPREPSTPHRYREGRTELRRDKSPGRPLEREKSPGRMLSTRRERSPGRLFEDSSRGRLPAGAVRTPLSQV
NKVWDQSSV

The disclosed NOV3b amino acid sequence has 638 of 647 amino acid residues (98%) identical to, and 643 of 647 amino acid residues (99%) similar to, the KIAA 1531 PROTEIN of 1060 amino acid residue prekallikrein-like protein from Homo sapiens (BAA96055) (E=0.0).

NOV3b is expressed primarily in normal brain but not in other normal tissues. Lower expression is seen in several tumor types.

NOV3b also has homology to the amino acid sequences shown in the BLASTP data listed in Table 3E.

TABLE 3E
BLAST results for NOV3b

| Gene Index/ | Protein/ | Length | Identity | Positives | |
|---|---|---|---|---|---|
| Identifier | Organism | (aa) | (%) | (%) | Expect |
| gi\|14768010\|ref\|XP_{—} | citron (rho- | 883 | 849/883 | 849/883 | 0.0 |
| 045786.1\| | interacting, | | (96%) | (96%) | |
| | serine/threonine | | | | |
| | kinase | | | | |
| | 21) [Homo | | | | |
| | sapiens] | | | | |
| gi\|6225217\|sp\|O14578\| | CITRON | 1286 | 1165/1286 | 1165/1286 | 0.0 |
| CTRO_HUMAN | PROTEIN | | (90%) | (90%) | |
| gi\|4589542\|dbj\|BAA76793.1\| | KIAA0949 | 940 | 887/940 | 887/940 | 0.0 |
| | protein | | (94%) | (94%) | |
| | [Homo | | | | |
| | sapiens] | | | | |
| gi\|3360514\|gb\|AAC27933.1\| | Citron-K | 1641 | 1476/1683 | 1490/1683 | 0.0 |
| | kinase [Mus | | (87%) | (87%) | |
| | musculus] | | | | |
| gi\|1345860\|sp\|P49025\| | CITRON | 1597 | 1427/1589 | 1442/1589 | 0.0 |
| CTRO_MOUSE | PROTEIN | | (89%) | (89%) | |

The homology of these sequences is shown graphically in the ClustalW analysis shown in Table 3F.

[image]

[image]

[image]

[image]

[image]

[image]

Tables 3G-K list the domain description from DOMAIN analysis results against NOV3b. This indicates that the NOV3 sequence has properties similar to those of other proteins known to contain this domain.

TABLE 3G

Domain Analysis of NOV3b
gnl|Pfam|pfam00780, cNH, CNH domain. Domain found in NIK1-like kinase,
mouse citron and yeast ROM1, ROM2. Unpublished observations
(SEQ ID NO: 71)
CD-Length = 304 residues, 99.7% aligned
Score = 238 bits (607), Expect = 2e−63

| | | | |
|---|---|---|---|
| Query: | 1619 | DMNCTLPFSDQ--VVLVGTEEGLYALNVLKN--SLTHVPGIGAVFQIYIIKDLEKLLMIA | 1674 |
| | | \| \| + +\|\|\|\|\|\|\|\|\| \|\| +\| + +\| \|\|+++++ \|\|\|\|+ | |
| Sbjct: | 2 | TAKCNHPITCDAKNLLVGTEEGLYVLNRSDQGGTLEKIISRRSVTQIWVLEENNVLIMIS | 61 |
| Query: | 1675 | GE--ERALCLVDVKICVKQSLAQSHLPAQPDISPNIFEAVKGCHLFGAGKIENGLCICAA | 1731 |
| | | \|+ \| \| +++ \| +\| + \| + ++ \| \|\|\|\|\|\|\| + \| +\|\|\| | |
| Sbjct: | 62 | GKICPYLYAHPLSGLREKDALGSARLVIRKNVWVICIEDVKGCHLFAVVNGKRILFLCAA | 119 |
| Query: | 1732 | MPSKVVIL-RYNENLSKYCIR-----KEIETSEPCSCIHFTNY---SILIGTNKFYEIDM | 1782 |
| | | +\|\| \| +\| \|\| + \| \| + ++ \| \| +\| \|+ | |
| Sbjct: | 120 | LPSSVQLLQWYNPLKXFKLFKSKFLKKLIVPVPLFVLLTSSSFELPKICIGVDK-NGFDV | 178 |
| Query: | 1783 | KQYTLEEFLDKNDHSLAPAVFAASSNSFPVSIVQVNSAGQREEYLLCFHEFGVFVDSYG- | 1841 |
| | | \|+ +\| \| \|\| \| \|\| \| + +\| \|\|\|\| \|\|\|\|\|\|+ \| | |
| Sbjct: | 179 | VQFHQTSLVSKEDLSLPNLNEETSKKPIPVIQVPQSD----DELLLCFDEFGVFVNLQGM | 234 |
| Query: | 1842 | RRSRTDDLKWSRLPLAFAYREPYLFVTHFNSLEVIEIQARSSAGTPARAYLDIPNPRYLG | 1901 |
| | | \|\|\|\| \| \| +\| \|\|\| \|\|\|\| \| \| +\| \|+ \| + \| \|\| | |
| Sbjct: | 235 | RRSRKPILTWEFMPEYFAYHEPYLLAFHSNGIEIRSIETGELLQELADR--EARKIRVLG | 292 |
| Query: | 1902 | PAISSGAIYLASSY | 1915 |
| | | \| \| +\|\| | |
| Sbjct: | 293 | S--SDRKILVSSSP | 304 |

TABLE 3H

Domain Analysis of NOV3b
gnl|Smart|smart00220, S_TKc, Serine/Threonine protein kinases,
catalytic domain; Phosphotransferases. Serine or threonine-specific
kinase subfamily. (SEQ ID NO: 72)
CD-Length = 256 residues, 100.0% aligned
Score = 230 bits (587), Expect = 5e−61

| | | | |
|---|---|---|---|
| Query: | 97 | FEVRSLVGCGHFAEVQVVREKATGDIYAMKVMKKKALLAQEQVSFFEEERNILSRSTSPW | 156 |
| | | +\|+ ++\| \| \| +\| + \|+\| \|\| \|+\|\|+\|\|+ \| +++ \| \|\| + \| | |
| Sbjct: | 1 | YELLEVLGKGAFGKVYLARDKKTGKLVAIKVIKKEKLK-KKKRERILREIKILKKLDHPN | 59 |
| Query: | 157 | IPQLQYAFQDRNHLYLVMEYQPGGDLLSLLNRYEDQLDENLIQFYLAELILAVHSVNLMG | 216 |
| | | \| +\| \|+\| + \|\|\|\|\|\|\| \|\|\|\| \|\| + +\| \|+ +\|\| +++ \|+ +\| \| | |
| Sbjct: | 60 | IVKLYDVFEDDDKLYLVMEYCEGGDLFDLLKKR-GRLSEDEARFYARQILSALEYLHSQG | |
| Query: | 217 | YVHRDIKPENILVDRTGMIKLVDFGSAAKMNSNXVNAKLPIGTPDYMAPEVLTVMNGDGK | 276 |
| | | +\|\|\|+\|\|\|\|\|\|+\| \|\|+\|\| \|\|\| \| +++\| +\|\|\|+\|\|\|\|\|\|\| | |
| Sbjct: | 119 | IIHRDLKPENILLDSDGHVKLADFGLAKQLDSGGTLLTTFVGTPEYMAPEVLL------G | 172 |
| Query: | 277 | GTYGLDCDWWSVGVIAYEMIYGRSPFAEGTSARTFNNIMNFQRFLKFPDDPKVSSDFLDL | |
| | | \|\| \| \|\|+\|\|\| \|\|+ \|+ \|\| + \| + +\| + \|\| | |
| Sbjct: | 173 | KGYGKAVDIWSLGVILYELLTGKPPFPGDDQLLALFKKIGKPPPPEPPPEWKISPEAKDL | 232 |
| Query: | 337 | IQSLLC-GQKERLKFEGLCCHPFF | 355 |
| | | \|+ \|\| ++\|\| \| \|\|\|\| | |
| Sbjct: | 233 | IKKLLVKDPEKRLTAEEALEHPFF | 256 |

TABLE 31

Domain Analysis of NOV3b
gnl|Smart|smart00036, cNH, Domain found in NIK1-like kinases, mouse
citron and yeast ROM1, ROM2; Unpublished observations.
(SEQ ID NO: 73)
CD-Length = 301 residues, 99.7% aligned
Score = 226 bits (577), Expect = 8e−60

| | | | |
|---|---|---|---|
| Query: | 1619 | DMNCTLPFSDQ--VVLVGTEEGLYALNVLKN--SLTHVPGIGAVFQIYIIKDLEKLLMIA | 1674 |
| | | \| + ++\|\|\|\|\|\|\|\|\| \|\|+ +\| + \| \| \|\|+++++ \|\|\|\|+ | |
| Sbjct: | 2 | TAKWNHPITCDAKILLVGTEEGLYVLNISDQHGTLEKLIGRRSVTQIWVLEENNVLLMIS | 61 |
| Query: | 1675 | GEERALC---LVDVKKVKQSLAQSHLPAQPDISPNIFEAVKGCHLFGAGKIENGLCICAA | 1731 |
| | | \|++ \| \| + + \| +\| + \| + ++ \|\|\|\|\|\| + \| +\| \| | |
| Sbjct: | 62 | GKKPQLYSHPLSALTEKDALGSARLVIRICNVLTK-IPDVKCCMLCAVVNGKRILFLCHA | 119 |
| Query: | 1732 | MPSKVVIL-RYNTENLSKYCIR-----KEIETSEPCSCIHFNY---SILIGTNKFYEIDM | 1782 |
| | | + \| \|\|+\| \|\| + \| + + \| \|++\| \|+ | |
| Sbjct: | 120 | LQSSVVLLQWYNPLKKFKLFKSKFLPPLISPVPVFVELVSSSFELPGICIGSDK-NGGDV | 178 |
| Query: | 1783 | KQYTLEEFLDKNDHSLAPAVFAASSNSFPVSIVQVNSAGQREEYLLCFHEFGVFVDSYG- | 1841 |
| | | \|+ + \| \| \|\| \|\| \| \| +\| \|\|\|+ \|\|\|\|\|\|+ \| | |
| Sbjct: | 179 | VQFH-QSLVSKEDLSLPFLSEETSSKPISVVQVP------ADELLLCYDEFGVFVNLYGM | 231 |
| Query: | 1842 | RRSRTDDLKWSRLPLAFAYREPYLFVTHFNSLEVIEIQARSSAGTPARAYLDIPNPRYLG | |
| | | \|\|\|\| \| \| +\| +\|\|\| \|\|\| \| \| +\|+ +\| \| \| \|\| | |
| Sbjct: | 232 | RRSRNPILHWEFMPESFAYXSPYLLAFEDNGIEIRSIKTGELLQELADR--KTRKIRLLG | 289 |
| Query: | 1902 | PAISSGAIYLASSY | 1915 |
| | | \| \| \|+\|\| | |
| Sbjct: | 290 | S--SDRKILLSSSP | 301 |

TABLE 3J

Domain Analysis of NOV3b
gnl|Pfam|pfam00069, pkinase, Protein kinase domain. (SEQ ID NO: 74)
CD-Length = 256 residues, 100.0% aligned
Score = 189 bits (481), Expect = 1e−48

| | | | |
|---|---|---|---|
| Query: | 97 | FEVRSLVGCGHFAEVQVVREKATGDIYAMKVMKKKALLAQEQVSFFEEERNILSRSTSPW | 156 |
| | | +\|+ +\| \| \| +\| + \| \|\|+\| \|+\|++\|\|+\| \| \| \| \|\| \| + \| | |
| Sbjct: | 1 | YELGEKLGSGAFGKVYXGKHKDTGEIVAIKILKKRSL-SEKICKRFLREIQILRRLSHPN | 58 |
| Query: | 157 | IPQLQYAFQDKNHLYLVMEYQPGGDLLSLLNRYEDQLDENLIQFYLAELILAVHSVHLMG | 216 |
| | | \| +\| \|++ +\|\|\|\|\|\|\|\| \|\|\|\| \| \| \| \| + +++ \| \| | |
| Sbjct: | 59 | IVRLLGVFEEDDHLYLVMEYNEGGDLFDYLRRNGLLLSEKEAKKIALQILRGLEYLHSRG | 118 |
| Query: | 217 | YVHRDIKPENILVDRTGHIKLVDFGSAAKMNS-NKVNAKLPIGTPDYMAPEVLTVMNGDC | 275 |
| | | \|\|\|\|+\|\|\|\|\|\|+\| \| \| \|\|\| \| \| \| ++ +\|\|\|+\|\|\|\|\|\|\| \| \| | |
| Sbjct: | 119 | IVHRDLKPENILLDENGTVKIADFGLARKLESSSYEKLTTFVGTPEYNAPEVL---EGRG | 175 |
| Query: | 276 | KGTYGLDCDWWSVGVIAYEMIYGRSPF-AEGTSARTFNNIMNFQRFLKFPDDPKVSSDFL | 334 |
| | | \| \| \|\|+\|\|\| \|\|++ \|+ \|\| \| + \| \| \| \|++ | |
| Sbjct: | 176 | ---YSSKVDVWSLGVILYELLTGKLPFPGIDPLEELFRIKERPRLRLPLP--PNCSEELK | 230 |
| Query: | 335 | DLIQSLLCGQ-KERLKFEGLCCHPPF | 359 |
| | | \|\|\|+ \| ++\| \|\|+\| | |
| Sbjct: | 231 | DLIKKCLNKDPEKRPTAKEILNHPWF | 256 |

TABLE 3K

Domain Analysis of NOV3b
gnl|Smart|smart00219, TyrKc, Tyrosine kinase, catalytic domain;
Phosphotransferases. Tyrosine-specific kinase subfamily.
(SEQ ID NO: 75)
CD-Length = 258 residues, 95.0% aligned
Score = 91.7 bits (226), Expect = 4e−19

| | | | |
|---|---|---|---|
| Query: | 104 | GCGHFAEVQVVREKATGDIYAMKVMKKKALLAQE-QVSFFEEERNILSRSTSPWIPQLQY | 162 |
| | | \| \| \| \|\| \| \| + +\| \| \| \|+ \| \| ++ + \| \| +\| | |
| Sbjct: | 8 | GEGAFGEVYKGTLKGKGGVEVEVAVKTLKEDASEQQIEEFLREARLMRRLDHPNIVKLLG | 67 |
| Query: | 163 | AFQDKNHLYLVMEYQPGGDLLSLLNRYEDQLDENLIQFYLAELI-LAVHSVHLMGYVHRD | 221 |
| | | ++ \| +\|\|\|\| \|\|\|\|\| \|++ + \| \| + + +\|\|\|\| | |
| Sbjct: | 68 | VCTEEEPLMIVMEYMEGGDLLDYLRKNRPKELSLSDLLSFALQIARGMEYLESKNFVHRD | 127 |
| Query: | 222 | IKPENILVDRTGHIKLVDFGSAAKMNSNKVNAKLPIGTPD--YMAPEVLTVMNGDGKGTY | 279 |
| | | \| \|\| +\|+ \|\|\| \| + + \| +\|\|\|\| \| \| + | |
| Sbjct: | 128 | LAARNCLVGENKTVKIADFGLARDLYDDDYYRKKKSPRLPIRWMAPESLK------DGKF | 181 |
| Query: | 280 | GLDCDWWSVGVIAYEMI-YGRSPFAEGTSARTFNNIMNFQRELKFPDDPKVSSDFLDLIQ | 338 |
| | | \| \|\| \|\|+ +\| \| \|\|+ ++ \| + \|+ \| + \|\|+ | |
| Sbjct: | 182 | TSKSDVWSFGVLLWEIFTLGESPYPGMSNEEVLEYLKKGYRLPQPPNCP---DEIYDLML | 238 |
| Query: | 339 | SLLCGQ---KERLEFE | 351 |
| | | \| ++\| \| | |
| Sbjct: | 239 | Q--CWAEDPEDRPTFS | 252 |

Recent data shows the identification of a novel serine/threonine kinase belonging to the myotonic dystrophy kinase family (DiCunto et al. Eur J Immunol December 2000;30(12):3403-10.). The kinase can be produced in at least two different isoforms: a approximately 240-kDa protein (Citron Rho-interacting kinase, CRIK), in which the kinase domain is followed by the sequence of Citron, a previously identified Rho/Rac binding protein; a approximately 54-kDa protein (CRIK-short kinase (SK)), which consists mostly of the kinase domain. CRIK and CRIK-SK proteins are capable of phosphorylating exogenous substrates as well as of autophosphorylation, when tested by in vitro kinase assays after expression into COS7 cells. CRIK kinase activity is increased several fold by coexpression of costitutively active Rho, while active Rac has more limited effects. Kinase activity of endogenous CRIK is indicated by in vitro kinase assays after immunoprecipitation with antibodies recognizing the Citron moiety of the protein. When expressed in keratinocytes, full-length CRIK, but not CRIK-SK, localizes into corpuscular cytoplasmic structures and elicits recruitment of actin into these structures. The previously reported Rho-associated kinases ROCK I and II are ubiquitously expressed. In contrast, CRIK exhibits a restricted pattern of expression, suggesting that this kinase may fulfill a more specialized function in specific cell types.

T cell receptor (TCR) engagement increases integrin-mediated adhesion to APC, resulting in the stabilization of the T cell. APC interaction and the close apposition of the two cell membranes. Engagement of either the TCR or CD3 chimeras with immobilized antibodies causes the rapid spreading of T cells in an integrin-independent fashion (Borroto et al. Eur J Immunol November 1999;29(11):3609-20). This effect concurs with the polymerization of the actin cytoskeleton and is dependent on the integrity of the immunoreceptor tyrosine-based activation motifs of the CD3 subunits. Expression of a dominant negative mutant of RhoA, as well as the Rho-specific inhibitor C3 toxin, abolished TCR-induced spreading. In contrast, constitutively active or dominant negative forms of Rac and Cdc42 did not affect cell spreading. Signals emanating from the TCR can directly induce T cell spreading, independently of integrins, and via a Rho-dependent reorganization of the actin cytoskeleton.

Motile lymphocytes adopt a polarized morphology with different adhesion molecules (ICAM, CD43 and CD44) and ERM actin-binding proteins concentrated on the uropod, a slender posterior appendage with important functions in cell-cell interactions and lymphocyte recruitment. The role of Rho family of GTPases (Rho, Rac and Cdc42) in the control of lymphocyte polarity and migration has been studied by analyzing the effects of exogenously introduced Rho GTPase mutants. Transfection of T cell lines that constitutively display a polarized motile morphology with activated mutants of RhoA, Rac1 and Cdc42 impaired cell polarization. A guanosine nucleotide exchange factor for Rac, Tiam-1, induced the same effect as activated Rac 1. Conversely, dominant negative forms of the three GTP-binding proteins induced a polarized phenotype in constitutively round-shaped T cells with redistribution of ICAM-3 and moesin to the uropod in an integrin-dependent manner. On the other hand, overexpression of dominant negative Cdc42 and activated mutants of all three Rho GTPases significantly inhibited SDF-1alpha-induced T cell chemotaxis. Together, these data demonstrate that Rho GTPases regulate lymphocyte polarization and chemokine-induced migration, and underscore the key role of Cdc42 in lymphocyte directional migration.

Activated Rho GTPases trigger distinctive kinase cascades. In particular, ROCK binds to Rho, and its kinase activity is moderately stimulated by this association. The citron molecule (Madaule et al., 1995), a specific interactor of Rho and Rac, shares a significant degree of structural homology with ROCK; however, its lack of a kinase domain raised the question of its biologic function. By PCR of a mouse primary keratinocyte cDNA library, Di Cunto et al. (1998) identified a novel serine/threonine kinase, CRIK (citron Rho-interacting kinase), belonging to the myotonic dystrophy kinase family. CRIK can be expressed as at least 2 isoforms, one of which encompasses the previously reported form of citron in almost its entirety. The long form of CRIK is a 240-kD protein in which the kinase domain is followed by the sequence of citron. The short form, CRIK-SK (short kinase), is an approximately 54 kD protein that consists mostly of the kinase domain. CRIK and CRIK-SK proteins are capable of phosphorylating exogenous substrates as well as of autophosphorylation, when tested by in vitro kinase assays after expression into COS-7 cells. CRIK kinase activity is increased several-fold by coexpression of constitutively active Rho, while active Rac has more limited effects. Kinase activity of the endogenous CRIK is indicated by in vitro kinase assays after immunoprecipitation with antibodies recognizing the citron moiety of the protein. When expressed in keratinocytes, full-length CRIK, but not CRIK-SK, localizes into corpuscular cytoplasmic structures and elicits recruitment of actin into these structures. The previously reported Rho-associated kinases ROCK1 and ROCK2 are ubiquitously expressed. Northern blot analysis of mouse tissues revealed a restricted pattern of expression limited to keratinocytes, brain, spleen, lung, kidney, and an especially strong signal in testis. No expression was detectable in heart, liver, or skeletal muscle. The CRIK protein contains a kinase domain, a coiled-coil domain, a leucine-rich domain, a Rho-Rac binding domain, a zinc finger region, a pleckstrin homology domain, and a putative SH3-binding domain. Di Cunto et al. (1998) reported cloning the human homolog of the CRIK kinase domain. They stated that the human homolog of citron is contained within a PAC clone (GenBank GENBANK AC002563) mapping to chromosome 12q. By screening size-fractionated human brain cDNA libraries for cDNAs encoding proteins larger than 50 kD, Nagase et al. (1999) identified CRIK as cDNA KIAA0949 (GenBank GENBANK AB023166). Di Cunto et al. (1998) mapped the human CRIK gene to chromosome 12q24.1-q24.3.

Di Cunto et al. (2000) generated mice deficient in citron kinase by targeted disruption. Citron-K−/− mice grow at slower rates, are severely ataxic, and die before adulthood as a consequence of fatal seizures. Their brains display defective neurogenesis, with dramatic depletion of microneurons in the olfactory bulb, hippocampus, and cerebellum. These abnormalities arise during development of the central nervous system due to altered cytokinesis and massive apoptosis. Di Cunto et al. (2000) concluded that citron-K is essential for cytokinesis in vivo, in specific neuronal precursors only. Moreover, they suggested a novel molecular mechanism for a subset of human malformation syndromes of the central nervous system.

The disclosed NOV3 nucleic acid of the invention encoding a RHO/RAC-interacting citron kinase-like protein includes the nucleic acid whose sequence is provided in Table 3A or 3C or a fragment thereof. The invention also includes a mutant or variant nucleic acid any of whose bases may be changed from the corresponding base shown in Table 3A or 3C while still encoding a protein that maintains its RHO/RAC-interacting citron kinase-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 10% percent of the bases may be so changed.

The disclosed NOV3 protein of the invention includes the RHO/RAC-interacting citron kinase-like protein whose sequence is provided in Table 3B or 3D. The invention also includes a mutant or variant protein any of whose residues may be changed from the corresponding residue shown in Table 3B or 3D while still encoding a protein that maintains its RHO/RAC-interacting citron kinase-like activities and physiological functions, or a functional fragment thereof. In the mutant or variant protein, up to about 13% percent of the residues may be so changed.

The protein similarity information, expression pattern, and map location for the RHO/RAC-interacting citron kinase-like protein and nucleic acid (NOV3) disclosed herein suggest that NOV3 may have important structural and/or physiological functions characteristic of the citron kinase-like family. Therefore, the NOV3 nucleic acids and proteins of the invention are useful in potential diagnostic and therapeutic applications. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo.

The NOV3 nucleic acids and proteins of the invention are useful in potential diagnostic and therapeutic applications implicated in various diseases and disorders described below. For example, the compositions of the present invention will have efficacy for treatment of patients suffering from asthma, arthritis, psoriasis, diabetes, and IBD, which require activated T cells, as well as diseases such as systemic lupus erythematosus that involve B cell activation, Autoimmune disease, Renal artery stenosis, Interstitial nephritis, Glomerulonephritis, Polycystic kidney disease, Renal tubular acidosis, IgA nephropathy, Hypercalceimia, Lesch-Nyhan syndrome, Von Hippel-Lindau (VHL) syndrome, Alzheimer's disease, Stroke, Tuberous sclerosis, hypercalceimia, Parkinson's disease, Huntington's disease, Cerebral palsy, Epilepsy, Multiple sclerosis, Ataxia-telangiectasia, Leukodystrophies, Behavioral disorders, Addiction, Anxiety, Pain, Neuroprotection, Endocrine dysfunctions, Obesity, Growth and Reproductive disorders Hemophilia, hypercoagulation, idiopathic thrombocytopenic purpura, allergies, immunodeficiencies, transplantation, Lymphaedema, Hemophilia, Hypercoagulation, Idiopathic thrombocytopenic purpura, Immunodeficiencies, Graft vesus host, Hirschsprung's disease, Crohn's Disease, Appendicitis Inflammatory bowel disease, Diverticular disease, and/or other pathologies. The NOV3 nucleic acid, or fragments thereof, may further be useful in diagnostic applications, wherein the presence or amount of the nucleic acid or the protein are to be assessed.

NOV3 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV3 protein have multiple hydrophilic regions, each of which can be used as an immunogen. In one embodiment, contemplated NOV3 epitope is from about amino acids 1 to 20. In another embodiment, a NOV3 epitope is from about amino acids 40 to 45. In additional embodiments, NOV3 epitopes are from about amino acids 110 to 150, from about amino acids 210 to 300, from about amino acids 410 to 900, from about amino acids 950 to 1200, from about amino acids 1250 to 1300, from about amino acids 1310 to 1450, from about amino acids 1490 to 1520, from about amino acids 1650 to 1680, from about amino acids 1800 to 1820, from about amino acids 1900 to 1920 and from about amino acids 1980 to 2053. This novel protein also has value in development of powerful assay system for functional analysis of various human disorders, which will help in understanding of pathology of the disease and development of new drug targets for various disorders.

NOV4

A disclosed NOV4 nucleic acid of 5691 nucleotides (designated CuraGen Acc. No. 105827550_EXT) encoding a novel Plexin-like protein is shown in Table 4A. An open reading frame was identified beginning with an ATG initiation codon at nucleotides 1-3 and ending with a TGA codon at nucleotides 5683-5685. A putative untranslated region downstream from the termination codon is underlined in Table 4A, and the start and stop codons are in bold letters.

TABLE 4A
NOV4 Nucleotide Sequence (SEQ ID NO: 12)

ATGAAAGCCATGCCCTGGAACTGGACCTGCCTTCTCTCCCACCTCCTCATGCTGGGCATGGGCTCCTCCA
CTTTGCTCACCCGGCAGCCAGCCCCGCTGTCCCAGAAGCAGCGGTCATTTGTCACATTCCGAGGAGAGCC
CGCCGAGGGTTTCAATCACCTGGTGGTGGATGAGAGGACAGGACACATTTACTTCGGGGCCGTCAATCGG
ATTTACAAGCTCTCCAGCGACCTGAAGGTCTTGGTGACGCATGAGACAGGGCCGGACGAGGACAACCCCA
AGTGTTACCCACCCCGCATCGTCCAGACCTGCAATGAGCCCCTGACCACCACCAACAATGTCAACAAGAT
GCTCCTCATAGACTACAAGGAGAACAGGCTGATTGCCTGTGGGAGCCTGTACCAAGGCATCTGCAAGCTG
CTGAGGCTGGAGGACCTCTTCAAGCTGGGGGAGCCTTATCATAAGAAGGAGCACTATCTGTCAGGTGTCA
ACGAGAGCGGCTCAGTCTTTGGAGTGATCGTCTCCTACAGCAACCTGGATGACAAGCTGTTCATTGCCAC
GGCAGTGGATGGGAAGCCCGAGTATTTTCCCACCATCTCCAGCCGGAAACTGACCAAGAACTCTGAGGCG
GATGGCATGTTCGCGTACGTCTTCCATGATGAGTTCGTGGCCTCGATGATTAAGATCCCTTCGGACACCT
TCACCATCATCCCTGACTTTGATATCTACTATGTCTATGGTTTTAGCAGTGGCAACTTTGTCTACTTTTT
GACCCTCCAACCTGAGATGGTGTCTCCACCAGGCTCCACCACCAAGGAGCAGGTGTATACATCCAAGCTC
GTGACGCTTTGCAAGGAGGACACAGCCTTCAACTCCTATGTAGAGGTGCCCATTGGCTGTGAGCGCAGTG
GGGTGGAGTACCGCCTGCTGCAGGCTGCCTACCTGTCCAAAGCGGGGGCCGTGCTTGGCAGGACCCTTGG
AGTCCATCCAGATGATGACCTGCTCTrCACCGTCTTCTCCAAGGGCCAGAAGCGGAAAATGAAATCCCTG
GATGAGTCGCCCCTGTGCATCTTCATCTTGAAGCAGATAAATGACCGCATTAAGGAGCGGCTGCAGTCTT
GTTACCGGGGCGAGGGCACGCTGGACCTGGCCTGGCTCAAGGTGAAGGACATCCCCTGCAGCAGTGCGCT
CTTAACCATTGACGATAACTTCTGTGGCCTGGACATGAATGCTCCCCTGGGAGTGTCCGACATGGTGCGT
GGAATTCCCGTCTTCACGGAGGACAGGGACCGCATGACGTCTGTCATCGCATATGTCTACAAGAACCACT
CTCTGGCCTTTGTGGGCACCAAAAGTGGCAAGCTGAAGAAGATCCGGGTGGATGGACCCAGGGGCAACGC
CCTCCAGTATGAGACGGTGCAGGTGGTGGACCCCGGCCCAGTCCTCCGGGATATGGCCTTCTCCAAGGAC
CACGAGCAACTCTACATCATGTCAGAGAGGCAGCTCACCAGAGTCCCTGTGGAGTCCTGTGGTCAGTATC
AGAGCTGCGGCGAGTGCCTTGGCTCAGGCGACCCCCACTGTGGCTGGTGTGTGCTGCACAACACGTGCAC
CCGGAAGGAGCGGTGTGAGCGGTCCAAGGAGCCCCGCAGGTTTGCCTCGGAGATGAAQCAGTGTGTCCGG
CTGACGGTCCATCCCAACAATATCTCCGTCTCTCAGTACAACGTGCTGCTGGTCCTGGAGACGTACAATG
TCCCGGAGCTGTCAGCTGGCGTCAACTGCACCTTTGAGGACCTGTCAGAGATGGATGGGCTGGTCGTGGG
CAATCAGATCCAGTGCTACTCCCCTGCAGCCAAGGAGGTGCCCCGGATCATCACAGAGAATGGGGACCAC
CATGTCGTACAGCTTCAGCTCAAATCAAAGGAGACCGGCATGACCTTCGCCAGCACCAGCTTTGTCTTCT
ACAATTGCAGCGTCCACAATTCGTGCCTGTCCTGCGTGGAGAGTCCATACCGCTGCCACTGGTGTAAATA
CCGGCATGTCTGCACCCATGACCCCAAGACCTGCTCCTTCCAGGAAGGCCGAGTGAAGCTGCCCGAGGAC
TGCCCCCAGCTGCTGCGAGTGGACAAGATCCTGGTGCCCGTGGAGGTGATCAAGCCTATCACGCTGAAGG
CCAAGAACCTCCCCCAGCCCCAGTCTGGGCAGCGTGGCTACGAATGCATCCTCAACATTCAGCGCAGCGA
GCAGCGAGTGCCCGCCCTGCGCTTCAACAGCTCCAGCGTACAGTGCCAGAACACCTCTTATTCCTATGAA
GGGATGGAGATCAACAACCTGCCCGTGGAGTTGACAGTCGTGTGGAATGGGCACTTCAACATTGACAACC
CAGCTCAGAATAAAGTTCACCTCTACAAGTGTGGAGCCATGCGTGAGAGCTGCGGGCTGTGCCTCAAGGC
TGACCCAGACTTCGCATGTGGCTGGTGCCAGGGCCCAGGCCAGTGCACCCTGCGCCAGCACTGCCCTGCC
CAGGAGAGCCAGTGGCTGGAGCTGTCTGGTGCCAAAAGCAAGTGCACAAACCCCCGCATCACAGAGATAA
TCCCGGTGACAGGCCCCCGGGAAGGGGGCACCAAGGTCACTATCCGAGGGGAGAACCTGGGCCTGGAATT
TCGCGACATCGCCTCCCATGTCAAGGTTGCTGGCGTGGAGTGCAGCCCTTTAGTGGATGGTTACATCCCT
GCAGAACAGATCGTGTGTGAGATGGGGGAGGCCAAGCCCAGCCAGCATGCAGGCTTCGTGGAGATCTGCG
TGGCTGTGTGTCGGCCTGAATTCATGGCCCGCTCCTCACAGCTCTATTACTTCATGACACTGACTCTCTC
AGATCTGAAGCCCAGCCGGGGGCCCATGTCCGGAGGGACCCAAGTGACCATCACAGGCACCAACCTGAAT
GCCGGAAGCAACGTGGTGGTGATGTTTGGAAAGCAGCCCTGTCTCTTCCACAGGCGATCTCCATCCTACA
TTGTCTGCAACACCACATCCTCAGATGAGGTCCTAGAGATGAAGGTGTCGGTGCAGGTGGACAGGGCCAA
GATCCACCACGACCTGGTCTTTCAGTATGTGGAAGACCCCACCATCGTGCGGATTGAGCCAGAATGGAGC
ATTGTCAGTGGAAACACACCCATCGCCGTATGGGGGACCCACCTGGACCTCATACAGAACCCCCAGATCC
GTGCCAACCATGGAGGGAACGACCACATCAATATCTGTGAGGTTCTGAACGCTACTGAGATGACCTGTCA
GGCGCCCGCCCTCGCTCTGCGTCCTGACCACCAGTCAGACCTGACCGAGAGGCCCGAGGAGTTTGGCTTC
ATCCTGGACAACGTCCAGTCCCTGCTCATCCTCAACAAGACCAACTTCACCTACTATCCCAACCCGGTGT
TTGAGGCCTTTGGTCCCTCAGGAATCCTGGAGCTCAAGCCTGGCACGCCCATCATCCTAAAGGGCAAGAA
CCTGATCCCGCCTGTGGCTGGCGGCAACGTGAAGCTGAACTACACTGTGCTGGTTGGGGAGAAGCCGTCC
ACCGTGACCGTGTCAGATGTCCAGCTGCTCTGCGAGTCCCCCAACCTCATCGGCAGGCACAAAGTGATGG
CCCGTGTCGGTGGCATGGAGTACTCCCCGGGGATGGTGTACATTGCCCCGGACAGCCCGCTCAGCCTGCC
CGCCATCGTCAGCATCGCAGTGGCTGGCGGCCTCCTCATCATTTTCATCGTGGCCGTGCTCATTGCCTAT
AAACGCAAGTCCCGCGAAAGTGACCTCACGCTGAAGCGGCTGCAGATGCAGATGGACAACCTGGAGTCCC
GTGTGGCCCTGGAGTGCAAGGAAGCCTTTGCCGAGCTGCAGACGGACATCCATGAGCTGACCAGTGACCT
GGATGGAGCCGGGATTCCGTTCCTGGACTATAGAACTTACACCATGCGGGTGCTGTTCCCAGGAATTGAA
GACCACCCTGTCCTCCGGGACCTTGAGGTCCCGGGCTACCGGCAGGAGCGTGTGGAGAAAGGCCTGAAGC
TCTTCGCCCAGCTCATCAACAACAAGGTGTTCCTGCTGTCCTTCATCCGCACGCTTGAGTCCCAGCGTAG
CTTCTCCATGCGCGACCGTGGCAACGTGGCCTCACTCATCATGACCGTGCTGCAGAGCAAGCTGGAGTAC
GCCACTGATGTGCTGAAGCAGCTGCTGGCCGACCTCATTGACAAGAACCTGGAGAGCAAGAACCACCCTA
AGCTGCTGCTCACGAGGACTGAGTCAGTGGCTGAGAAGATGCTGACCAATTGGTTTACTTTCCTCCTCTA
CAAGTTCCTCAAGGAGTGTGCTGGGGAGCCCCTCTTCTCCCTGTTCTGTGCCATCAAGCAGCAGATGGAG
AAGGGCCCCATTGACGCCATCACGGGCGAGGCCCGCTACTCCTTGAGCGAGGACAAGCTCATCCGCCAGC
AGATTGACTACAAAACCCTGGTCCTGAGCTGTGTCAGCCCAGACAATGCCAACAGCCCCGAGGTCCCAGT
AAAGATCCTCAACTGTGACACCATCACTCAGGTCAAGGAGAAGATTCTGGATGCCATCTTCAAGAATGTG
CCTTGCTCCCACCGGCCCAAAGCTGCAGATATGGATCTGGAGTGGCGACAAGGAAGTGGGGCAAGGATGA
TCTTGCAGGATGAAGACATCACCACCAAGATTGAGAATGATTGGAAGCGACTGAACACACTGGCCCACTA
CCAGGTGCCAGATGGTTCCGTGGTGGCATTAGTGTCCAAGCAGGTGACAGCCTATAACGCAGTGAACAAC
TCCACCGTCTCCAGGACCTCAGCAAGTAAATATGAAAACATGATCCGGTACACGGGCAGCCCCGACAGCC
TCCGCTCACGGACACCTATGATCACTCCTGACCTGGAGAGTGGAGTCAAGATGTGGCACCTAGTGAAGAA
CCACGAGCACGGAGACCAGAAGGAGGGGGACCGGGGGAGCAAGATGGTGTCTGAAATCTACCTGACCCGA
CTCCTGGCCACTAAGGGCACACTGCAGAAGTTTGTGGATGACCTCTTTGAGACCATCTTCAGCACGGCAC
ACCGTGGCTCTGCCCTGCCCCTGCCATCAAGTACATGTTTGACCTTCCTGGATGAGCAGGCTGATAAACA
TGGCATTCATGACCCGCACGTCCGCCATACCTGGAAGAGCAATTGCCTGCCCCTGAGGTTTTGGGTCAAC
ATGATCAAGAACCCGCAGTTTGTGTTTGACATCCATAAGAACAGCATCACAGACGCCTGCCTCTCTGTGG
TGGCTCAGACCTTCATGGACTCTTGCTCCACGTCAGAGCACCGGCTGGGCAAGGACTCGCCCTCCAACAA
GCTGCTGTATGCCAAGGACATCCCCAGCTACAAGAATTGGGTGGAGAGGTATTACTCAGACATAGGGAAG
ATGCCAGCCATCAGCGACCAAGACATGAACGCATACCTGGCTGAGCAGTCCCGGATGCACATGAATGAGT
TCAACACCATGAGTGCACTCTCAGAGATCTTCTCCTATGTGGGCAAATACAGCGAGGAGATCCTTGGACC
TCTGGACCACGATGACCAGTGTGGGAAGCAGAAACTGGCCTACAAACTAGAACAAGTCATAACCCTCATG
AGCTTAGACAGCTGAAATAAA

The nucleic acid sequence of NOV4, localized on chromosome 7, has 4004 of 5567 bases (71%) identical to a plexin-2 mRNA from mouse (GENBANK-ID: D86949) (E=0.0).

A NOV4 polypeptide (SEQ ID NO: 13) encoded by SEQ ID NO: 12 is 1896 amino acid residues and is presented using the one letter code in Table 4B. Signal P, Psort and/or Hydropathy results predict that NOV4 has no signal peptide and is likely to be localized at the plasma membrane with a certainty of 0.46. In other embodiments, NOV4 may also be localized to the endoplasmic reticulum (membrane) with a certainty of 0.1000, the endoplasmic reticulum (lumen) with a certainty of 0.1000, or the outside with a certainty of 0.1000.

TABLE 4B

NOV4 protein sequence (SEQ ID NO: 13)

MKAMPWNWTCLISHLLMVGMGSSTLLTRQPAPLSQKQRSFVTFRGEPAEGFNHLVVDERTGHIYLGAVNRIYKL
SSDLKVLVTHETGPDEDNPKCYPPRIVQTCNEPLTTTNNVNKMLLIDYKENRLIACGSLYQGICKLLRLEDLFK
LGEPYHKKEHYLSGVNESGSVFGVIVSYSNLDDKLFIATAVDGKPEYFPTISSRKLTKNSEADGMFAYVFHDEF
VASMIKIPSDTFTIIPDFDIYYVYGFSSGNFVYFLTLQPEMVSPPGSTTKEQVYTSKLVRLCKEDTAFNSYVEV
PIGCERSGVEYRLLQAAYLSKAGAVLGRTLGVHPDDDLLFTVFSKGQKRKNKSLDESALCIFILKQINDRIKER
LQSCYRGEGTLDLAWLKVKDIPCSSALLTIDDNFCGLDMNAPLGVSDMVRGIPVFTEDRDRNTSVIAYVYKNHS
LAFVGTKSGKLKKIRVDGPRGNALQYETVQVVDPGPVLRDMAFSKDHEQLYIMSERQLTRVPVESCGQYQSCGE
CLGSGDPHCGWCVLHNTCTRKERCERSKEPRRPASEMKQCVRLTVHPNNISVSQYNVLLVLETYNVPELSAGVN
CTFEDLSEMDGLVVGNQIQCYSPAAKEVPRIITENGDHHVVQLQLKSKETGMTFASTSFVFYNCSVHNSCLSCV
ESPYRCHWCKYRHVCTHDPKTCSFQEGRVKLPEDCPQLLRVDKILVPVEVIKPITLKAKNLPQPQSGQRGYECI
LNIQGSEQRVPALRFNSSSVQCQNTSYSYEGMEINNLPVELTVVWNGMFNIDNPAQNKVHLYKCGANRESCGLC
LKADPDFACGWCQGPGQCThRQHCPAQESQWLELSGAKSKCTNPRITEIIPVTGPREGGTKVTIRGENLGLEFR
DIASHVKVAGVECSPLVDGYIPAEQIVCEMGEAKPSQHAGFVEICVAVCRPEFMARSSQLYYFMTLTLSDLKPS
RGPMSGGTQVTITGTNIMAGSNVVVNFGKQPCLFHRRSPSYIVCNTTSSDEVLEMKVSVQVDRAKIHQDLVPQY
VEDPTIVRIEPEWSIVSGNTPIAVWGTHLDLIQNPQIRAKHGGKEHINICEVLNATENTCQAPALALGPDHQSD
LTERPEEFGFILDNVQSLLILNKTNFTYYPNPVFEAFGPSGILELKPGTPIILKGKNLIPPVAGGNVKLNYTVL
VGEKPCTVTVSDVQLLCESPNLIGRHKVMARVGGMEYSPGMVYIAPDSPLSLPAIVSIAVAGGLLIIFIVAVLI
AYKRKSRESDLTLKRLQMQMDNLESRVALECKEAFAELQTDIHELTSDLDGAGIPFLDYRTYTMRVLFPGIEDH
PVLRDLEVPGYRQERVEKGLKLFAQLINNKVFLLSFIRTLESQRSFSMRDRGNVASLIMTVLQSKLEYATDVLK
QLLADLIDKNLESKNHPKLLLRRTESVAEKMLTNWFTFLLYKFLKECAGEPLFSLFCAIKQQMEKGPIDAITGE
ARYSLSEDKLIRQQIDYKTLVLSCVSPDNANSPEVPVKILNCDTITQVKEKILDAIFKNVPCSHRPKAADMDLE
WRQGSGARMILQDEDITTKIENDWKRLNTLAHYQVPDGSVVALVSKQVTAYNAVNNSTVSRTSASKYENMIRYT
GSPDSLRSRTPMITPDLESGVKMWHLVKNHEHGDQKEGDRGSKMVSEIYLTRLLATKGTLQKFVDDLFETIFST
AHRGSALPLAIKYMFDFLDEQADKHGIMDPHVRHTWKSNCLPLRFWVNMIKNPQFVFDIHKNSITDACLSVVAQ
TFMDSCSTSEHRLGKDSPSNKLLYAKDIPSYKNWVERYYSDIGKMPAISDQDMNAYLEAQSRMHMNEFNTMSAL
SEIFSYVGKYSEEILGPLDHDDQCGKQKLAYKILEQVITLMSLDSNK

The full amino acid sequence of the protein of the invention was found to have 1263 of 1857 amino acid residues (68%) identical to, and 1501 of 1857 amino acid residues (80%) similar to, the 1884 amino acid residue plexin-2 protein from mouse (SPTREMBL-P70207) (E=0.0), and 364 of 801 amino acid residues (45%) identical to, and 520 of 801 amino acid residues (64%) similar to, the 2135 amino acid residue Human Plexin protein (patp:AAU00019) (E=2.6⁻²⁸³).

The disclosed NOV4 protein is expressed in at least the following tissues: fibroblast like synoviocytes (normal), fetal brain, adipose, microvascular endothelial cells-lung, thalamus, fetal cerebral cortex, temporal lobe, parietal lobe, fetal cerebellum, and testis. TaqMan expression data for NOV4 is shown below in Example 2.

NOV4. also has homology to the amino acid sequences shown in the BLASTP data listed in Table 4C.

TABLE 4C
BLAST results for NOV4

| Gene Index/ | | Length | Identity | Positives | |
|---|---|---|---|---|---|
| Identifier | Protein/Organism | (aa) | (%) | (%) | Expect |
| gi\|13649119\|ref\|XP_{—} | SEX gene [Homo | 1871 | 1121/1846 | 1413/1846 | 0.0 |
| 010150.2\| | sapiens] | | (60%) | (75%) | |
| gi\|3413888\|dbj\|BAA32308.1\| | KIAA0463 protein | 1963 | 1270/1870 | 1508/1870 | 0.0 |
| | [Homo sapiens] | | (67%) | (79%) | |
| gi\|2134135\|pir\|\|I51553 | Plexin - African | 1905 | 1220/1915 | 1468/1915 | 0.0 |
| | clawed frog | | (63%) | (75%) | |
| gi\|14424639\|gb\|AAH09343.1\| | Unkown(protein for | 813 | 641/810 | 717/810 | 0.0 |
| AAH09343 | IMAGE: 4130636) | | (79%) | (88%) | |
| | [Homo sapiens] | | | | |
| gi\|10047165\|dbj\|BAB13376.1\| | KIAA1550 protein | 593 | 513/513 | 513/513 | 0.0 |
| | [Homo sapiens] | | (100%) | (100%) | |

The homology of these sequences is shown graphically in the ClustalW analysis shown in Table 4D.

[image]

[image]

[image]

[image]

[image]

[image]

[image]

Tables 4E-K list the domain description from DOMAIN analysis results against NOV4. This indicates that the NOV4 sequence has properties similar to those of other proteins known to contain this domain.

TABLE 4E

Domain Analysis of NOV4
gnl|Smart|smart00630, Sema, semaphorin domain (SEQ ID NO: 76)
CD-Length = 430 residues, 100.0% aligned
Score = 226 bits (575), Expect = 1e−59

| | | | |
|---|---|---|---|
| Query: | 51 | FNHLVVDERTGHIYLGAVNRIYKLSSDLKVLVTHETGPDEDNPKCYPPRIVQTCNEPLTT | 110 |
| | | +\|++\|\| \| +\|+\|\| \|\|+\| \|\| +\| +\|\|\| +\| \| | |
| Sbjct: | 1 | LQNLLLDEDNGTLYVGARNRLYVLSLNLISEAEVKTGPVLSSPDCEECV----SKGKDPP | 56 |
| Query: | 111 | TNNVNKM-LLIDYKENRLIACGS-LYQGICKLLRLEDLFKLGEPYHKKEHYLSGVNESGS | 168 |
| | | \|+ \|\| + \|\|+\|\| + \|+ \|\|+ +\| +\|+\|+ \| +\| +\| \| + + + | |
| Sbjct: | 57 | TDCVNFIRLLLDYNADHLLVCGTNAFQPVCRLINLGNLDRL-EVGRESGRGRCPFDPQHN | 115 |
| Query: | 169 | VFGVIVSYSNLDDKLFIATAVD--GKPEYPPTISSRKLTKNSEADGMFAYVFHDEFVASM | 226 |
| | | \|+\| \| +\|++ \| \| \| \| + \| + \| | |
| Sbjct: | 116 | STAVLV-----DGELYVGTVADFSGSDPAIYRSLSVRRLKGT-------SGPSLRTVLYD | 163 |
| Query: | 227 | IKIPSDTFTIIPDFDIYYVYGFSSGNFVYFLTLQPENVSPPGSTTKEQVYTSKLVRLCKE | 286 |
| | | + + + +\|\| \| \|\|+\|\|\|\| + + + \|++ \|+\|\| | |
| Sbjct: | 164 | SRWLN---------EPNFVYAFESGDFVYPFFRETAVEDENC----GKAVVSRVARVCKM | 210 |
| Query: | 287 | DTA--------FNSYVEVPIGC---ERSGVEYRLLQAAYLSKAGAVLGRTLGVHPDDDLL | 335 |
| | | \| + \|+++ + \| + \|\|\|\|+\| \|\|+ \|\|+\| | |
| Sbjct: | 211 | DVGGPRSLSKKWTSFLKARLECSVPGEFPFYFNELQAAPLLPAGS---------ESDDVL | 261 |
| Query: | 336 | FTVFSKGQKRKMKSLDESALCIFILKQINDRIKERLQSCYRGEGTLDL----AWLKVKDI | 391 |
| | | + \|\|\| + \|\|+\| \| \| \|\| \| + \| \| + | |
| Sbjct: | 262 | YGVFSTSS----NPIPGSAVCAFSLSDINAVFNEPFKECETGNSQWLPYPRGLVPFPRPG | 317 |
| Query: | 392 | PC-----SSALLTIDDNFCGLDMNAPLGVSDMVRGIPVF--TEDRDRMTSV----IAYVY | 440 |
| | | \| \|\| \| \| \| + \| \|+\| \|+ +\|\|+ + | |
| Sbjct: | 318 | TCPNTPLSSKDLPDDVLNFIKTHPLMDEVVQPLTGRPLFVKTDSNYLLTSIAVDRVRTDG | 377 |
| Query: | 441 | KNHSLAFVGTKSGKLKKIRVDGPRGN--ALQYETVQVVDPGPVLRDMAFSKDH | 491 |
| | | \|+++ \|+\|\| \|++ \|+ + + ++ \| + \| \|\|\| + \|+ \| | |
| Sbjct: | 378 | GNYTVLFLGTSDGRILKVVLSRSSSSSESVVLEEISVFDPGSPVSDLVLSPKK | 430 |

TABLE 4F

Domain Analysis of NOV4
gnl|Pfam|pfam01403, Sema, Sema domain. The Sema domain occurs in
semaphorins, which are a large family of secreted and transmembrane
proteins, some of which function as repellent signals during axon
guidance. Sema domains also occur in the hepatocyte growth factor
receptor.
(SEQ ID NO: 77)
CD-Length = 433 residues, 100.0% aligned
Score = 186 bits (471), Expect = 1e−47

| | | | |
|---|---|---|---|
| Query: | 51 | FNHLVVDERTGHIYLGAVNRIYKLS----SDLKVLVTHETGPDEDNPKCYPPRIVQTCNE | 106 |
| | | \| \|++\|\| \| +\|+\|\| \|\|+\| \|+ \|++ \| \| \| \| +\| | |
| Sbjct: | 1 | FVTLLLDEDRGRLYVGARNRVYVLNLEDLSEVLNLKTGWPGSCETCEECNMKGKS----- | 55 |
| Query: | 107 | PLTTTNNVNKMLLIDYKENRLIACGS-LYQGICKLLRLEDLFKLGEPYHKKEMYLSGVNE | 165 |
| | | \|\|\| \| + +\| \| + \| \|\|+ +\| +\| \|+ \| \|\|\| \| + + | |
| Sbjct: | 56 | PLTECTNFIR-VLQAYNDTHLYVCGTNAFQPVCTLINLGDLFSLDVDNEEDGCGDCPYDP | 114 |
| Query: | 166 | SGSVFGVIVSYSNLDDKLFIATAVDGKPEYFPTISSRKLTKNSEADGMFAYVFHDEFVAS | 225 |
| | | \|+ \|+\| +\|+ \| +\| + + + + \|\|\| + | |
| Sbjct: | 115 | LGNTTSVLVQ----GGELYSGTVID------FSGRDPSIRRLLGSHDGLRTEFHD---SK | 161 |
| Query: | 226 | MIKIPSDTFTIIPDFDIYYVYGFSSGNFVYFLTLQPEMVSPPGSTTKEQVYTSKLVRLCK | 285 |
| | | + +\|+ + + \|+\|\|+ \| \| + \|\|\| + + \| \|++ \|+\|\| | |
| Sbjct: | 162 | WLNLPNFVDS----YPIHYVHSF-SDDKVYFFFRETAVEDSNCKT-----IHSRVARVCK | 211 |
| Query: | 286 | EDTAFNSYVEVPIG---------CERSGVE----YRLLQAAYLSKAGAVLGRTLGVHPDD | 332 |
| | | \| \|\|+\|+ \| \| + \|\|\|\|++ \|\| \| | |
| Sbjct: | 212 | NDPGGRSYLELNKWTTFLKARLNCSIPGEGTPFYFNELQAAFVLPTGA---------DTD | 262 |
| Query: | 333 | DLLFTVFSKGQKRKMKSLDESALCIFILKQIND--RIKERLQSCYRGEGTLDLAWLKVKD | 390 |
| | | +\|+ \|\|+ \| \|\|+\| \| + \|\| + \|\| + + | |
| Sbjct: | 263 | PVLYGVFTISS----NSSAGSAVCAFSMSDINQVFEGPFKHQSPNSKWLPYRGKVPQPRP | 318 |
| Query: | 391 | IPCSSA-LLTIDDNFCGLDMNAPLG--VSDMVRGIPVFTEDRDR-------MTSVIAYVY | 440 |
| | | \| +\| \| + \|+ \|\| \| + +\|+\| + \| \| | |
| Sbjct: | 319 | CQCPNASGLNLPDDTLNFIRCHPLMDEVVPPLHNVPLFVGQSGNYRLTSIAVDRVRAGDG | 378 |
| Query: | 441 | KNHSLAFVGTKSGKLKKIRVDGPRGNA-----LQYETVQVV-DPGPVLRDMAFSKD | 490 |
| | | + +++ \|+\|\| \|++ \| +\| \| ++ + \| \| \| \|\| \| + \|\|+ | |
| Sbjct: | 379 | QIYTVLFLGTDDGRVLK-QVVLSRSSSASYLVVVLEESLVFPDGEPVQRNVISSKN | 433 |

TABLE 4G

Domain Analysis of NOV4
gnl|Pfam|pfam01833, TIG, IPT/TIG domain. This family consists of a
domain that has an immunoglobulin like fold. These domains are found
in cell surface receptors such as Met and Ron as well as in
intracellular transcription factors where it is involved in DNA
binding. CAUTION: This family does not currently recognise a
significant number of members.
(SEQ ID NO: 78)
cD-Length = 85 residues, 98.8% aligned
Score = 69.7 bits (169). Expect = 1e−12

| | | | |
|---|---|---|---|
| Query: | 955 | TLSDLKPSRGPMSGGTQVTITGTNLNAGSNVVVMFGKQPCLFHRRSPSYIVCNTTSSDEV | 1014 |
| | | ++ + \|\| \|\|+\|\|\|\|++\|\|\|\|+\|\| +\| ++ \| \|\| \| + \| \|\|\| \| | |
| Sbjct: | 2 | VITSISPSSGPLSGGTEITITGSNLGSGEDIKVTFGGTECDVVSQEASQIVCKTPPYANG | 61 |
| Query: | 1015 | LEMKVSVQVDRA-KIHQDLVFQYV | 1037 |
| | | \|+\| +\| + \| \|\| | |
| Sbjct: | 62 | GPQPVTVSLDGGGLSSSPVTFTYV | 85 |

TABLE 4H

Domain Analysis of NOV4
gnl|Pfam|pfam01833, TIG, IPT/TIG domain. This family consists of a
domain that has an immunoglobulin like fold. These domains are found
in cell surface receptors such as Met and Ron as well as in
intracellular transcription factors where it is involved in DNA
binding. CAUTION: This family does not currently recognise a
significant number of members.
(SEQ ID NO: 79)
CD-Length = 85 residues, 91.8% aligned
Score = 54.3 bits (129), Expect = 6e−08

| | | | |
|---|---|---|---|
| Query: | 858 | PRITEIIPVTGPREGGTKVTIRGENLGLEFRDIASHVKVAGVECSPLVDGYIPAEQIVCE | 917 |
| | | \| \|\| \| \| +\|\| \|\|\|++\|\| \| \|\|\| \| \| \|\| + \|\|\|\|+ | |
| Sbjct: | 1 | PVITSISPSSGPLSGGTEITITGSNLGSGED---IKVTFGGTECDVVSQEA---SQIVCK | 54 |
| Query: | 918 | MGE-AKPSQHAGFVEICVAVCRPE | 940 |
| | | \| \| + | |
| Sbjct: | 55 | TPPYANGGPQPVTVSLDGGGLSSS | 78 |

TABLE 41

Domain Analysis of NOV4
gnl/Pfam/pfam01833, TIG, IPT/TIG domain. This family consists of a
domain that has an immunoglobulin like fold. These domains are found
in cell surface receptors such as Met and Ron as well as in
intracellular transcription factors where it is involved in DNA
binding. CAUTION: This family does not currently recognise a
significant number of members.
(SEQ ID NO: 80)
CD-Length = 85 residues, 100.0% aligned
Score = 45.8 bits (107), Expect = 2e−05

| | | | |
|---|---|---|---|
| Query: | 1040 | PTIVRIEPEWSIVSGNTPIAVWGTHLDLIQNPQIRAKHGGKEHINICEVLN--ATEMTCQ | 1097 |
| | | \| \| \| \| +\|\| \| \| + \|++\| ++ \|+ \|\| \| \|+\|++ \|+++ \|+ | |
| Sbjct: | 1 | PVITSISPSSGPLSGGTEITITGSNLGSGED--IKVTFGGTE----CDVVSQEASQIVCK | 54 |
| Query: | 1098 | APALALGPDHQSDLTERPEEFGFILDNVQSLLILNKTNFTYY | 1139 |
| | | \| \| \|+ \|\| \| + \|\|\| | |
| Sbjct: | 55 | TPPYA---------NGGPQPVTVSLDGGG--LSSSPVTFTYV | 85 |

TABLE 4J

Domain Analysis of NOV4
gnl|Smart|smart00423, PSI, domain found in Plexins.
Semaphorins and Integrins
(SEQ ID NO: 81)
CD-Length = 47 residues. 100.0% aligned
Score = 46.6 bits (109), Expect = 1e−05

| | | | |
|---|---|---|---|
| Query: | 655 | NCSVHNSCLSCVESPYR-CHWCKYRHVCTHDPKTCSFQEGRVKLPEDCP | 702 |
| | | \|\| + \|\| \|+ + \| \|\| + \|\| \| \|\| | |
| Sbjct: | 1 | RCSAYTSCSECLLARDPYCAWCSSQGRCTSGE-RCDSLRQNW-SSGQCP | 47 |

TABLE 4K

Domain Analysis of NOV4
gnl|Smart|smart00429, IPT, ig-like, plexins, transcription factors
(SEQ ID NO: 82)
CD-Length = 93 residues, 100.0% aligned
Score = 57.8 bits (138), Expect = 6e−09

| | | | |
|---|---|---|---|
| Query: | 1039 | DPTIVRIEPEWSIVSGNTPIAVWGTHLDLIQNPQIRAKHGGKEHINICEVLNATEMTCQA | 1098 |
| | | \|\| \| \|\| \| +\|\| \| \| + \| +\|\| \| + \| + ++ \| + \|+ | |
| Sbjct: | 1 | DPVITRISPNSGPLSGGTRITLCGKNLDSISVVFVEVGVGEVPCTFLPSDVSQTAIVCKT | 60 |
| Query: | 1099 | PALALGPDHQSDLTERPEEFGFILDNVQSLLILNKTNFTYY | 1139 |
| | | \| \| \| \| \| + \|\|\| | |
| Sbjct: | 61 | PPYHNIP----GSVPVRVEVGLRNGGVPGE----PSPFTYV | 93 |

Semaphorins are a large family of secreted or cell-bound signals, known to guide axons in developing nervous tissue. They are expressed in a variety of adult and embryonic tissues and are thought to have a broader spectrum of functions. Recent evidence suggests that semaphorins and their receptors play a key role in the control of cellular interactions, most likely in cell-cell repulsion (Tamagnone and Comoglio. Trends Cell Biol September 2000;10(9):377-83.). A subset of semaphorins interacts with neuropilins—cell-surface molecules lacking a signalling-competent cytoplasmic domain. Another large family of transmembrane molecules, namely plexins, bind specifically to semaphorins. Thus plexins, alone, or in association with neuropilins, behave as fully functional semaphorin receptors. The intracellular responses elicited by plexins are unknown, but their large cytoplasmic moiety, containing the strikingly conserved sex-plexin (SP) domain, is likely to trigger novel signal-transduction pathways.

Chemorepulsive signals such as the semaphorins play an essential role in navigating axons over large distances in the developing nervous system. The effects of one of these repulsive cues, semaphorin 3A (Sema3A), are mediated by the membrane protein neuropilin-1 (Npn-1). Recent work has shown that neuropilin-1 is essential but not sufficient to form functional Sema3A receptors and indicates that additional components are required to transduce signals from the cell surface to the cytoskeleton (Rohm et al. Mech Dev May 2000;93(1-2):95-104). It has been shown that members of the plexin family interact with the neuropilins and act as co-receptors for Sema3A. Neuropilin/plexin interaction restricts the binding specificity of neuropilin-1 and allows the receptor complex to discriminate between two different semaphorins. Deletion of the highly conserved cytoplasmic domain of Plexin-A1 or -A2 creates a dominant negative Sema3A receptor that renders sensory axons resistant to the repulsive effects of Sema3A when expressed in sensory ganglia. These data suggest that functional semaphorin receptors contain plexins as signal-transducing and neuropilins as ligand-binding subunits.

Class 1 and 3 semaphorins repulse axons but bind to different cell surface proteins. Two known semaphorin-binding proteins, plexin 1 (Plex 1) and neuropilin-1 (NP-1), form a stable complex (Strittmatter. Cell 1999 Oct. 1;99(l):59-69.). Plex 1 alone does not bind semaphorin-3A (Sema3A), but the NP-1/Plex 1 complex has a higher affinity for Sema3A than does NP-1 alone. While Sema3A binding to NP-1 does not alter nonneuronal cell morphology, Sema3A interaction with NP-1/Plex 1 complexes induces adherent cells to round up. Expression of a dominant-negative Plex 1 in sensory neurons blocks Sema3A-induced growth cone collapse. Sema3A treatment leads to the redistribution of growth cone NP-1 and plexin into clusters. Thus, physiologic Sema3A receptors consist of NP-1/plexin complexes.

As mentioned previously, the semaphorin family of molecules contains members known to deliver guidance cues to migrating axons during development. Semaphorins also have been identified on the surface of hematopoietic cells and, interestingly, in the genomes of certain lytic viruses. Recent studies indicate that semaphorins bind with high affinity to at least two different receptor families and are biologically active on immune cells as well as neuronal cells (Spriggs. Curr Opin Immunol August 1999;11(4):387-91.).

The mammalian olfactory system is capable of discriminating among a large variety of odor molecules and is therefore essential for the identification of food, enemies and mating partners. The assembly and maintenance of olfactory connectivity have been shown to depend on the combinatorial actions of a variety of molecular signals, including extracellular matrix, cell adhesion and odorant receptor molecules (Pasterkamp et al. Cell Mol Biol September 1999;45(6):763-79). Recent studies have identified semaphorins and their receptors as putative molecular cues involved in olfactory pathfinding, plasticity and regeneration. Neuropilins were shown to serve as receptors for secreted class 3 semaphorins, whereas members of the plexin family are receptors for class 1 and V (viral) semaphorins.

In Drosophila, plexin A is a functional receptor for semaphorin-1a. The human plexin gene family comprises at least nine members in four subfamilies (Goodman et al. Cell 1999 Oct. 1;99(l):71-80.). Plexin-B1 is a receptor for the transmembrane semaphorin Sema4D (CD100), and plexin-C1 is a receptor for the GPI-anchored semaphorin Sema7A (Sema-K1). Secreted (class 3) semaphorins do not bind directly to plexins, but rather plexins associate with neuropilins, coreceptors for these semaphorins. Plexins are widely expressed: in neurons, the expression of a truncated plexin-A1 protein blocks axon repulsion by Sema3A. The cytoplasmic domain of plexins associates with a tyrosine kinase activity. Plexins may also act as ligands mediating repulsion in epithelial cells in vitro. Plexins are receptors for multiple (and perhaps all) classes of semaphorins, either alone or in combination with neuropilins, and trigger a novel signal transduction pathway controlling cell repulsion.

Plexin is a type I membrane protein which was identified in Xenopus nervous system by hybridoma technique. Molecular cloning studies demonstrated that the extracellular segment of the plexin protein possesses three internal repeats of cysteine cluster which are homologous to the cysteine-rich domain of the c-met proto-oncogene protein product. A cell aggregation test revealed that the plexin protein mediated cell adhesion via a homophilic binding mechanism, in the presence of calcium ions (Fujisawa et al. Dev Neurosci 1997;19(l):101-5.). Plexin was-expressed in the neuronal elements composing particular neuron circuits in Xenopus CNS and PNS. These findings indicate that plexin is a new member of the Ca(2+)-dependent cell adhesion molecules, and suggest that the molecule plays an important role in neuronal cell contact and neuron network formation.

Plexin (previously referred to as B2) is a neuronal cell surface molecule that has been identified in Xenopus. cDNA cloning reveals that plexin has no homology to known neuronal cell surface molecules but possesses, in its extracellular segment, three internal repeats of cysteine clusters that are homologous to the cysteine-rich domain of the c-met proto-oncogene protein product. The exogenous plexin proteins expressed on the surfaces of L cells by cDNA transfection mediate cell adhesion via a homophilic binding mechanism, under the presence of calcium ions (Fujisawa. Neuron June 1995;14(6):1189-99.). Plexin is expressed in the receptors and neurons of particular sensory systems. These findings indicate that plexin is a novel calcium-dependent cell adhesion molecule and suggest its involvement in specific neuronal cell interaction and/or contact.

The disclosed NOV4 nucleic acid of the invention encoding a Plexin-like protein includes the nucleic acid whose sequence is provided in Table 4A or a fragment thereof. The invention also includes a mutant or variant nucleic acid any of whose bases may be changed from the corresponding base shown in Table 4A while still encoding a protein that maintains its Plexin-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 29% percent of the bases may be so changed.

The disclosed NOV4 protein of the invention includes the Plexin-like protein whose sequence is provided in Table 4B. The invention also includes a mutant or variant protein any of whose residues may be changed from the corresponding residue shown in Table 4B while still encoding a protein that maintains its Plexin-like activities and physiological functions, or a functional fragment thereof. In the mutant or variant protein, up to about 40% percent of the residues may be so changed.

The protein similarity information, expression pattern, and map location for the Plexin-like protein and nucleic acid (NOV4) disclosed herein suggest that this NOV4 protein may have important structural and/or physiological functions characteristic of the Plexin family. Therefore, the NOV4 nucleic acids and proteins of the invention are useful in potential diagnostic and therapeutic applications. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo.

The NOV4 nucleic acids and proteins of the invention are useful in potential diagnostic and therapeutic applications implicated in various diseases and disorders described below. For example, the compositions of the present invention will have efficacy for treatment of patients suffering from Von Hippel-Lindau (VHL) syndrome, Alzheimer's disease, Stroke, Tuberous sclerosis, hypercalceimia, Parkinson's disease, Huntington's disease, Cerebral palsy, Epilepsy, Lesch-Nyhan syndrome, Multiple sclerosis, Ataxia-telangiectasia, Leukodystrophies, Behavioral disorders, Addiction, Anxiety, Pain, Neurodegeneration, Systemic lupus erythematosus, Autoimmune disease, Asthma, Emphysema, Scleroderma, allergy, ARDS, Obesity, Metabolic Dysregulation, Infertility, and/or other pathologies. The NOV4 nucleic acids, or fragments thereof, may further be useful in diagnostic applications, wherein the presence or amount of the nucleic acid or the protein are to be assessed.

NOV4 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example, the disclosed NOV4 protein has multiple hydrophilic regions, each of which can be used as an immunogen. In one embodiment, a contemplated NOV4 epitope is from about amino acids 20 to 30. In another embodiment, a NOV4 epitope is from about amino acids 180 to 190. In additional embodiments, NOV4 epitopes are from about amino acids 180 to 270, from about amino acids 310 to 320, from about amino acids 380 to 390, from about amino acids 400 to 405, from about amino acids 420 to 550, from about amino acids 620 to 630, from about amino acids 650 to 700, from about amino acids 790 to 900, from about amino acids 1040 to 1050, from about amino acids 1100 to 1120, from about amino acids 1220 to 1240, from about amino acids 1410 to 1420, from about amino acids 1450 to 1500, from about amino acids 1580 to 1600, from about amino acids 1620 to 1650, from about amino acids 1720 to 1730 and from about amino acids 1800 to 1900. These novel proteins can be used in assay systems for functional analysis of various human disorders, which will help in understanding of pathology of the disease and development of new drug targets for various disorders.

NOV5

A disclosed NOV5 nucleic acid of 1535 nucleotides (also referred to as GMAC027612_A) encoding a novel dopamine receptor-like protein is shown in Table 5A. An open reading frame was identified beginning with an ATG initiation codon at nucleotides 7-9 and ending with a TAA codon at nucleotides 1447-1449. A putative untranslated region upstream from the initiation codon and downstream from the termination codon is underlined in Table 5A, and the start and stop codons are in bold letters.

TABLE 5A
NOV5 Nucleotide Sequence (SEQ ID NO: 14)

CCCGAAATGCTGCCGCCAAGGAGCAACGACACCGCGTACCCGGGGCAGTTAGCGCTATACCA
GCAGCTGGCGCAGGGGAATGCCGTGGGGGGCTCGGCGGGGGCACCGCCACTGGGGCCCGTGC
AGGTGGTCACCGCCTGCCTGCTGACCCTACTCGTCATCTGGACCTTGCTGGGCAACGTGCTG
GTGTCCGCAGCCATCGTGTGGAGCCGCCACCTGCGCGCCAAGATGACCAACGTCTTCATCGT
GTCTCTACCTGTGTCAGACCTCTTCGTGGCGCTGCTGGTCATGTCCTGGAAGGCAGTCGCCG
AGGTGGCCGGTTACTGGCCCTTTGAAGCGTTCTGCGACGTCTGGGTGGCCTTCGACATCATG
TGCTCCACCGCCTCCATCCTGAACCTGTGCGTCATCAGCGTGGCCCGCTACTGGGCCATCTC
CAGGCCCTTCCGCTACGAGCGCAAGATGACCCAGCGCATGGCCTTGGTCATGGTCCGCCCGG
CCTGGACCTTGTCCAGCCTCATCTCCTTCATTCCGGTCCAGCTCAACTGGCACAGGGACCAG
GCGGTCTCTTGGGGTGGGCTGGACCTGCCAAACAACCTGGCCAACTGGACGCCCTGGGAGGA
GGCCGTTTGGGAGCCCGACGTGAGGGCAGAGAACTGTGACTCCAGCCTGAATCGAACCTACG
CCATCCCTTCCTCGCTCATCAGCTTCTACATCCCCATGGCCATCATGATCGTGACCTACACG
CGCATCTACCGCATCGCCCAGGTGCAGATCCGCAGGATTTCCTCCCTGGAGAGGGCCGCAGA
GCACGTGCAGAGCTGCCGGAGCAGCGCAGGCTGCACGCCCGACACCAGCCTGCGGTTTTCCA
TCAAGAAGGAGACCGAGGTTCTCAAGACCCTGTCGGTGATCATGGCGGTCTTCGTGTGTTGC
TGGCTGCCCTTCTTCATCCTTAACTGCATGGTTCCTTTCTGCAGTGGACACCCCAAAGGCCC
TCCGGCCGGCTTCCCCTGCGTCAGTGAGACCACATTCGATGTCTTCATCTGGTTCTGCTGGG
CCAACTCCTCACTCAACCCAGTCCCCAGTCACTATGCCTTCAACGCCGACTTCCGGAAGGTG
TTTGCCCAGCTGCTGGGGTGCAGCCACGTCTGCTCCCGCACGCCGGTGGAGACGGTGAACAT
CAGCAATGAGCTCATCTCCTACAACCAAGACACGGTCTTCCACAAGGAAATCGCAGCTGCCT
ACATCCACATGATGCCCAACGCCATTCCCCCCGGGGACCGGGAGGTGGACAACGATGAGGAG
GAGGAGAGTCCTTTCGATCGCATGTCCCAGATOTATCAGACATCCCCAGATGGTGACCATGT
TGCAGAGTCTGTCTGGGAGCTGGACTGCGAGGGGGAGATTTCTTTAGACAAAATAACACCTT
TCACCCCAAATGGATTCCATTAAACTGCATTAAGAAACCCCCTCATGGATCTGCATAACCAC
ACAGACATTGACAAGCATGCACACACAAGCAAATACATGGCTTTCCA

The NOV5 nucleic acid was identified on chromosome 4 and has 1494 of 1536 bases (97%) identical to a Human dopamine receptor (D5) transcribed pseudogene mRNA from Homo sapiens (GENBANK-ID: M75867) (E=0.0)

A disclosed NOV5 polypeptide (SEQ ID NO:15) encoded by SEQ ID NO:14 is 480 amino acid residues and is presented using the one-letter code in Table 5B. Signal P, Psort and/or Hydropathy results predict that NOV5 has a signal peptide and is likely to be localized in the plasma membrane with a certainty of 0.6400. In other embodiments, NOV5 may also be localized to the Golgi body with acertainty of 0.4600, the endoplasmic reticulum (membrane) with a certainty of 0.3700, or the endoplasmic reticulum (lumen) with a certainty of 0.1000. The most likely cleavage site for a NOV5 peptide is between amino acids 63 and 64, at: VSA-AI.

TABLE 5B
Encoded NOV5 protein sequence (SEQ ID NO: 15)

MLPPRSNDTAYPGQLALYQQLAQGNAVGGSAGAPPLGPVQVVTACLLTLLVIWTLLGNVLVSAAIVWSRHLR
AKMTNVFIVSLPVSDLFVALLVMSWKAVAEVAGYWPFEAFCDVWVAFDIMCSTASILNLCVISVARYWAISR
PFRYERKMTQRMALVMVRPAWTLSSLISFIPVQLNWHRDQAVSWGGLDLPNNLANWTPWEEAVWEPDVRAEN
CDSSLNRTYAIPSSLISFYIPMAIMIVTYTRIYRIAQVQIRRISSLERAAEHVQSCRSSAGCTPDTSLRFSI
KKETEVLKTLSVIMGVFVCCWLPFFILNCMVPFCSGHPKGPPAGFPCVSETTFDVFIWFCWANSSLNPVPSH
YAFNADFRKVFAQLLGCSHVCSRTPVETVNISNELISYNQDTVFHKEIAAAYIHMMPNAIPPGDREVDNDEE
EESPFDRMSQIYQTSPDGDHVAESVWELDCEGEISLDKITPFTPNGFH

The disclosed NOV5 amino acid sequence has 437 of 480 amino acid residues (91%) identical to, and 446 of 480 amino acid residues (92%) similar to, the 477 amino acid residue DOPAMINE RECEPTOR (D(5) DOPAMINE RECEPTOR) (D1BETA DOPAMINE RECEPTOR) protein from Homo sapiens (Human) (P21918) (E=3.3e⁻²³⁷).

NOV5 is expressed in at least the following tissues: fetal heart, pooled human melanocyte, fetal heart, and pregnant uterus. TaqMan data for NOV5 is shown below in Example 2.

NOV5 also has homology to the amino acid sequences shown in the BLASTP data listed in Table 5C.

TABLE 5C
BLAST results for NOV5

| Gene Index/ | | Length | Identity | Positives | |
|---|---|---|---|---|---|
| Identifier | Protein/Organism | (aa) | (%) | (%) | Expect |
| gi\|4503391\|ref\|NP_000789.1\| | dopamine receptor | 477 | 437/480 | 446/480 | 0.0 |
| | D5; Dopamine | | (91%) | (92%) | |
| | receptor D1B | | | | |
| | [Homo sapiens] | | | | |
| gi\|6978781\|ref\|NP_036900.1\| | D(1B) DOPAMINE | 475 | 376/480 | 403/480 | 0.0 |
| | RECEPTOR (D(5) | | (78%) | (83%) | |
| | DOPAMINE | | | | |
| | RECEPTOR) | | | | |
| gi\|1169230\|sp\|P42290\| | D(1B) DOPAMINE | 457 | 299/445 | 341/445 | e−158 |
| DBDR_XENLA | RECEPTOR (D(5) | | (67%) | (76%) | |
| | DOPAMINE | | | | |
| | RECEPTOR) | | | | |
| gi\|1362719\|pir\|\|B55886 | dopamine receptor | 486 | 321/483 | 353/483 | e−157 |
| | D1B - chicken | | (66%) | (72%) | |
| gi\|1518040\|gb\|AAC60070.1\| | dopamine D1B | 458 | 297/446 | 346/446 | e−154 |
| | receptor | | (66%) | (76%) | |
| | [Anguilla | | | | |
| | anguilla] | | | | |

The homology of these sequences is shown graphically in the ClustalW analysis shown in Table 5D.

[image]

[image]

[image]

Tables 5E list the domain description from DOMAIN analysis results against NOV5. This indicates that the NOV5 sequence has properties similar to those of other proteins known to contain this domain.

TABLE 5E

Domain Analysis of NOV5
gnl|Pfam|pfam00001, 7tm_1, 7 transmembrane receptor (rhodopsin
family),
(SEQ ID NO: 83)
CD-Length = 254 residues, 99.2% aligned
Score = 188 bits (478), Expect = 5e−49

| | | | |
|---|---|---|---|
| Query: | 57 | GNVLVSAAIVWSRHLRAKMTNVFIVSLPVSDLFVAILVMSWKAVAEVAGYWPF-EAFCDV | 115 |
| | | \|\|+\|\| \|+ ++ \|\| \|\|+\|+++\| \|+\|\| \| + \| \| \| \| \| +\| \| + | |
| Sbjct: | 1 | GNLLVILVILRTKKLR-TPTNIFLLNLAVADLLFLLTLPPWALYYLVGGDWVFGDALCKL | 59 |
| Query: | 116 | WVAFDIMCSTASILNLCVISVARYWAISRPFRYERKMTQRMALVMVRPAWTLSSLISFIP | 175 |
| | | \| ++ \|\|\|\| \| \|\|+ \|\| \|\| \| \|\| \| \| \| \| \|++ \| \|+ \|+\| \| | |
| Sbjct: | 60 | VGALFVVNGYASILLLTAISIDRYLAIVHPLRYRRIRTPRRAKVLILLVWVLALLLSLPP | 119 |
| Query: | 176 | VQLNWHRDQAVSWGGLDLPNNLANWTPWEEAVWEPDVRAENCDSSLNRTYAIPSSLISFY | 235 |
| | | + +\| \| \| + + \|+ \|+\| + \|+\|+ \| | |
| Sbjct: | 120 | LLFSWLR------------TVEEGNTTVCLIDFPEE--------SVKRSYVLLSTLVGFV | 159 |
| Query: | 236 | IPMAIMIVTYTRIYRIAQVQIRRISSLERAAEHVQSCRSSAGCTPDTSLRFSIKKETEVL | 295 |
| | | +\|+ +++\| \|\|\|\| \| + + \| \|\|+ \| + | |
| Sbjct: | 160 | LPLLVILVCYTRILRTLRKRARSQ----------------------RSLKRRSSSERKAA | 197 |
| Query: | 296 | KTLSVIMGVFVCCWLPFFILNCMVPFCSGHPKGPPAGFPCVSETTFDVFIWFCWANSSLN | 355 |
| | | \| \| \|++ \|\|\| \|\|\|\|+ \|+ + \| \| \| + +\| + \|\| \|\| | |
| Sbjct: | 198 | KMLLVVVVVFVLCWLPYHIVLLLDSLC-------LLSIWRVLPTALLITLWLAYVNSCLN | 250 |
| Query: | 356 | PV | 357 |
| | | \|+ | |
| Sbjct: | 251 | PI | 252 |

NOV5 also has homology to proteins found in the patp patent database as shown in Table 5E.

TABLE 5E
BLAST results for NOV5 for patp Database

| | | | | Posi- | |
|---|---|---|---|---|---|
| Gene Index/ | Protein/ | Length | Identity | tives | |
| Identifier | Organism | (aa) | (%) | (%) | Expect |
| patp: AAR22546 | Truncated | 479 | 453/480 | 460/480 | 1.7e−244 |
| | Dopamine | | (94%) | (95%) | |
| | D1 receptor | | | | |
| | encoded by | | | | |
| | pseudogene | | | | |
| | clone GL-39 | | | | |
| | Homo | | | | |
| | sapiens | | | | |
| patp: AAR79381 | Dopamine | 477 | 436/480 | 445/480 | 2.0e−236 |
| | receptor D5 - | | (90%) | (92%) | |
| | Homo | | | | |
| | sapiens | | | | |

The rhodopsin-like GPCRs themselves represent a widespread protein family that includes hormone, neurotransmitter and light receptors, all of which transduce extracellular signals through interaction with guanine nucleotide-binding (G) proteins. Although their activating ligands vary widely in structure and character, the amino acid sequences of the receptors are very similar and are believed to adopt a common structural framework comprising 7 transmembrane (TM) helices.

G-protein-coupled receptors (GPCRs) constitute a vast protein family that encompasses a wide range of functions (including various autocrine, paracrine and endocrine processes). They show considerable diversity at the sequence level, on the basis of which they can be separated into distinct groups. The term clan is used to describe the GPCRs, as they embrace a group of families for which there are indications of evolutionary relationship, but between which there is no statistically significant similarity in sequence. The currently known clan members include the rhodopsin-like GPCRs, the secretin-like GPCRs, the cAMP receptors, the fungal mating pheromone receptors, and the metabotropic glutamate receptor family.

The diverse physiologic actions of dopamine are mediated by its interaction with 2 types of G protein-coupled receptor, D1 and D2, which stimulate and inhibit, respectively, the enzyme adenylyl cyclase. Three groups reported the cloning of the D1 dopamine receptor gene (Dearry et al., 1990; Zhou et al., 1990; Sunahara et al., 1990 ). The gene encodes a protein of 446 amino acids having a predicted relative molecular mass of 49,300 and a transmembrane topology similar to that of other G protein-coupled receptors. Northern blot analysis and in situ hybridization showed that the mRNA for this receptor is most abundant in caudate, nucleus accumbens and olfactory tubercle, with little or no mRNA detectable in substantia nigra, liver, kidney, or heart (Dearry et al., 1990 ). Sunahara et al. (1990) reported that the DRD1 gene is intronless and, by Southern blot hybridization to DNAs from a hybrid cell panel, they mapped the gene to chromosome 5. Family linkage studies confirmed this assignment and suggested that it is in the same general region as the gene for glucocorticoid receptor and D5S22, a marker about 12 cM from GRL. This places it in the 5q31-q34 region near the structurally homologous genes for beta-2-adrenergic receptor and alpha-1-adrenergic receptor. Using pulsed field gel electrophoresis and a range of different restriction enzyme digests, Boultwood et al. (1991) established that GRL and DRD1 are on the same 300-kb genomic DNA fragment. Grandy et al. (1990) used the recently cloned DRD1 gene to map the locus to chromosome 5 in rodent-human somatic cell hybrids. Fluorescence in situ hybridization refined the localization to 5q35.1. A 2-allele EcoRI RFLP associated with DRD1 allowed confirmation of the localization by linkage analysis in CEPH families. The homologous gene in the mouse is located on chromosome 13.

The distal end of 5q, 5q31.1-qter, contains the genes for 2 adrenergic receptors, ADRB2 and ADRA1B and the dopamine receptor type 1A gene. Krushkal et al. (1998) used an efficient discordant sib-pair ascertainment scheme to investigate the impact of this region of the genome on variation in systolic blood pressure in young Caucasians. They measured 8 highly polymorphic markers spanning this positional candidate gene-rich region in 427 individuals from 55 3-generation pedigrees containing 69 discordant sib pairs, and calculated multipoint identity by descent probabilities. The results of genetic linkage and association tests indicated that the region between markers D5S2093 and D5S462 was significantly linked to 1 or more polymorphic genes influencing interindividual variation in systolic blood pressure levels. Since the ADRA1B and DRD1A genes are located close to these markers, the data suggested that genetic variation in 1 or both of these G protein-coupled receptors, which participate in the control of vascular tone, plays an important role in influencing interindividual variation in systolic blood pressure levels

The disclosed NOV5 nucleic acid of the invention encoding a Dopamine receptor-like protein includes the nucleic acid whose sequence is provided in Table 5A or a fragment thereof. The invention also includes a mutant or variant nucleic acid any of whose bases may be changed from the corresponding base shown in Table 5A while still encoding a protein that maintains its Dopamine receptor-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 10% percent of the bases may be so changed.

The disclosed NOV5 protein of the invention includes the Dopamine receptor-like protein whose sequence is provided in Table 5B. The invention also includes a mutant or variant protein any of whose residues may be changed from the corresponding residue shown in Table 5B while still encoding a protein that maintains its Dopamine receptor-like activities and physiological functions, or a functional fragment thereof. In the mutant or variant protein, up to about 34% percent of the residues may be so changed.

The NOV5 nucleic acids and proteins of the invention are useful in potential therapeutic applications implicated in schizophrenia, and other dopamine-dysfunctional states, Hypertension, Huntington's disease, levodopa-induced dyskinesias, alcoholism, Diabetes Insipidus and Mellitus with Optic Atrophy and Deafness, Wolfram Syndrome and/or other pathologies and disorders. For example, a cDNA encoding the dopamine receptor-like protein may be useful in gene therapy, and the dopamine receptor-like protein may be useful when administered to a subject in need thereof. By way of nonlimiting example, the compositions of the present invention will have efficacy for treatment of patients suffering from schizophrenia, and other dopamine-dysfunctional states, Hypertension, Huntington's disease, levodopa-induced dyskinesias, alcoholism, Diabetes Insipidus and Mellitus with Optic Atrophy and Deafness, Wolfram Syndrome, as well as other diseases, disorders and conditions. The NOV5 nucleic acid, or fragments thereof, may further be useful in diagnostic applications, wherein the presence or amount of the nucleic acid or the protein are to be assessed.

NOV5 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV5 protein have multiple hydrophilic regions, each of which can be used as an immunogen. In one embodiment, contemplated NOV5 epitope is from about amino acids 1 to 10. In other embodiments, NOV5 epitope is from about amino acids 125 to 150, from about amino acids 175 to 230, from about amino acids 250 to 300, from about amino acids 320 to 330, from about amino acids 350 to 370, from about amino acids 380 to 410, or from about amino acids 420 to 460. This novel protein also has value in development of powerful assay system for functional analysis of various human disorders, which will help in understanding of pathology of the disease and development of new drug targets for various disorders.

NOV6

A disclosed NOV6 nucleic acid of 2657 nucleotides (also referred to as GM523_e_{—}1_A) encoding a novel Metabotropic Glutamate Receptor-like protein is shown in Table 6A. An open reading frame was identified beginning with an ATG initiation codon at nucleotides 6-8 and ending with a TGA codon at nucleotides 2643-2645. A putative untranslated region upstream from the initiation codon and downstream from the termination codon is underlined in Table 6A, and the start and stop codons are in bold letters.

TABLE 6A
NOV6 Nucleotide Sequence (SEQ ID NO: 16)

GATTCATGAAGATGTTGACAAGACTACAAGTTCTTATGTTAGCTTTGTTTTCAAAGGGATTTTTAGTCTC
TTTAGGAGATCACAACTTTATGAGGAGAGAAATTAAAATAGAAGGAGACCTTGTTTTAGGGGGCTTATTT
CCTATTAATGAAAAAGGCACTGGAACTGAAGAGTGTGGACGAATCAATGAAGACAGAGGTATCCAACGcc
TGGAGGCCATGTTGTTTGCCATTGATGAAATCAACAAAGACAATTACTTGCTTCCAGGAGTGAAGCTGGG
GGTTCACATTTTGGATACATGTTCAAGAGACACCTATGCATTAGAGCAGTCACTGGAGTTTGTCAGAGCA
TCGTTGACTAAAGTGGATGAAGCTGAATATATGTGTCCTGATGGATCATATGCTATTCAAGAAAACATCC
CACTACTCATTGCAGGAGTCATTGGCGGTTCGTACAGCAGTGTTTCCATACAGGTAGCAAACCTGCTGAG
GCTCTTCCAGATCCCTCAGATAAGCTACGCCTCCACCAGTGCCAAACTCAGCGACAAATCGCGCTATGAT
TATTTTGCCAGGACCGTGCCCCCTGACTTCTACCAGGCCAAAGCCATGGCCGAGATCTTGCGCTACTTTA
ACTGGACCTATGTGTCCACTGTTGCCTCTGAAGGTGACTATGGGGAGACAGGGATTGAGGCCTTCGAGCA
GGAAGCAAGGCTACGCAACATCTGCATCGCCACTGCTGAAAAGGTCGGGCGCTCCAACATCCGCAAGTCC
TACGACAGCGTGATCCGTGAGCTCCTGCAGAAACCTAACGCGCGAGTTGTGGTCCTGTTCATGCGCAGTG
ATGACTCACGAGAGTTGATCGCTCCAGCCAGCCGCGTGAATGCTTCCTTCACCTGGGTGGCCAGCGATGG
CTGGGGTGCACAGGAGAGCATTGTCAAGGGCAGTGAGCACGTCGCCTATGGAGCCATCACCCTGGAGCTG
GCGTCCCACCCTGTTCGTCAGTTTGATCGCTACTTCCAGAGCCTCAACCCCTACAACAATCATCGTAACC
CCTGGTTCCGAGACTTCTGGGAGCAGAAGTTCCAGTGCAGCCTCCAGAACAAGAGAAACCACAGACAGAT
TTGTGACAAGCACCTGGCCATTGACAGCAGCAACTATGAACAAGAATCCAAGATCATGTTTGTGGTGAAT
GCAGTGTATGCCATGGCGCATGCGCTGCACAAAATGCAACGCACCCTCTGTCCCAACACCACCAAGCTCT
GTGATGCAATGAAGATCCTGGATGGAAAGAAGTTGTACAAAGATTATTTGCTGAAAATCAACTTCCTTGC
TCCATTCAACCCAAATAAAGGAGCAGACAGCATTGTGAAGTTTGACACTTACGGAGACGGGATGGGAACA
TACAACGTGTTCAACTTCCAGCATATAGGTGGAAAGTATTCCTACTTAAAAGTTGGCCACTGGGCAGAAA
CTTTATATCTAGATGTGGACTOTATTCATTGGTCCCGGAACTCAGTCCCCACTTCCCAGTGCAGTGATCC
CTGTGCCCCCAATGAAATGAAAAACATGCAGCCAGGAGATGTTTGCTGCTGGATCTGCATCCCATGTGAG
CCCTATGAATACCTGGTTGATGAGTTCACCTGCATGGATTGTGGCCCTGGCCAGTGGCCCACTGCAGACC
TATCTGGATGCTACAACCTTCCAGAGGATTACATCAGGTGGGAAGATGCCTGOGCAATAGGCCCAGTCAC
TATTGCCTGCCTGGGTTTTATGTGTACATGCATAGTCATAACTGTTTTTATCAAGCACAACAACACACCC
TTGGTCAAAGCATCAGGCCGAGAACTCTGCTACATCTTGTTATTTGGAGTTAGCCTGTCCTATTGCATGA
CATTCTTCTTCATTGCThAGCCATCGCCTGTCATCTGTGCATTGCGCCGACTTGGGCTTGGGACCTCCTT
TGCCATCTGTTATTCAGCTCTCCTGACCAAGACAAACTGCATCGCTCGCATCTTTGATGGGGTCAAGAAT
GGCGCTCAGAGGCCAAAATTCATCAGCCCCAGTTCTCAGGTTTTTATCTGCCTGGGTTTGATACTGGTGC
AAATTGTGATGGTGTCTGTGTGGCTTATCTTGGAGACTCCAGGTACTAGAAGATACACCCTGCCAGAGAA
GCGGGAAACAGTCATCCTAAAATGCAATGTCAAAGATTCCAGCATGTTGATCTCTCTGACCTATGACGTG
GTTCTGGTGATTCTATGCACTGTGTATGCCTTCAAAACAAGGAAGTGTCCTGAAAACTTCAATGAAGCCA
AGTTCATAGGCTTCACCATGTACACCACCTGCATCATCTCGTTGGCATTCCTCCCTATATTTTATGTGAC
ATCAAGTGACTACAGAGTACAGACGACAACAATGTGCATCTCCGTTAGCTTGAGTGGTTTCGTGGTCTTG
GGCTGTTTGTTTGCCCCCAAGGTGCACATTGTCCTGTTCCAACCCCAGAAGAATGTGGTCACACACAGAC
TTCACCTCAACAGGTTCAGTGTCAGTGGAACTGCGACCACATATTCTCAGGCCTCTCCAAGCACGTATGT
GCCAACGGTGTGCAATGGGCGGGAAGTCCTCGACTCCACCACCTCATCTCTGTGATTGTGAATTGCA

The disclosed NOV6 nucleic acid sequence has 2522 of 2658 bases (94%) identical to a Rat metabotropic glutamate receptor 3 mRNA from Rattus norvegicus (GENBANK-ID: M92076) (E=0.0).

A disclosed NOV6 polypeptide (SEQ ID NO:17) encoded by SEQ ID NO:16 is 879 amino acid residues and is presented using the one-letter amino acid code in Table 6B. Signal P, Psort and/or Hydropathy results predict that NOV6 contains a signal peptide and is likely to be localized in the endoplasmic reticulum (membrane) with a certainty of 0.6850. In other embodiments, NOV6 is also likely to be localized to the plasma membrane with a certainty of 0.6400, to the Golgi body with a certainty of 0.4600, or to the endoplasmic reticulum (lumen) with a certainty of 0.1000. The most likely cleavage site for a NOV6 peptide is between amino acids 24 and 25, at: SLG-DH.

TABLE 6B
Encoded NOV6 protein sequence (SEQ ID NO: 17).

MKMLTRLQVLMLALFSKGFLVSLGDHNFMRREIKIEGDLVLGGLFPINEKGTGTEECGRINEDRGIQRLEAMLFA
IDEINKDNYLLPGVKLGVHILDTCSRDTYALEQSLEFVRASLTKVDEAEYMCPDGSYAIQENIPLLIAGVIGGSY
SSVSIQVANLLRLFQIPQISYASTSAKLSDKSRYDYFARTVPPDFYQAKAMAEILRYFNWTYVSTVASEGDYGET
GIEAFEQEARLRNICIATAEKVGRSNIRKSYDSVIRELLQKPNARVVVLFMRSDDSRELIAAASRVNASFTINAS
DGWGAQESIVKGSEMVAYGAITLELASHPVRQFDRYFQSLNPYNNHRNPWFRDFWEQKFOCSLQNKRNHRQICDK
HLAIDSSNYEQESKIMFVVNAVYAMAHALHKMQRTLCPNTTKLCDAMKILDGKKLYKDYLLKINFLAPFNPNKGA
DSIVKFDTYGDGMGRYNVFNFQHIGGKYSYLKVGHWAETLYLDVDSIHWSRNSVPTSQCSDPCAPNEMKNMQPGD
VCCWICIPCEPYEYLVDEFTCMDCGPGQWPTADLSGCYNLPEDYIRWEDAWAIGPVTIACLGFMCTCIVITVFIK
HNNTPLVKASGRELCYILLFGVSLSYCMTFFFIAKPSPVICALRRLGLGTSFAICYSALLTKTNCIARIFDGVKN
GAQRPKFISPSSQVFICLGLILVQIVMVSVWLILETPGTRRYTLPEKRETVILKCNVKDSSMLISLTYDVVLVIL
CTVYAFKTRKCPENFNEAKFIGFTMYFTCIIWLAFLPIFYVTSSDYRVOTTTMCISVSLSGFVVLGCLFAPKVHI
VLFQPQKNVVTHRLMLNRFSVSGTATTYSQASASTYVPTVCNGREVLDSTTSSL

The disclosed NOV6 amino acid sequence has 877 of 879 amino acid residues (99%/o) identical to, and 878 of 879 amino acid residues (99%) similar to, the 879 amino acid residue METABOTROPIC GLUTAMATE RECEPTOR 3 PROTEIN protein from Mus musculus (Mouse (Q9QYS2) (E=0.0).

NOV6 also has homology to the amino acid sequences shown in the BLASTP data listed in Table 6C.

TABLE 6C
BLAST results for NOV6

| Gene Index/ | | Length | Identity | Positives | |
|---|---|---|---|---|---|
| Identifier | Protein/Organism | (aa) | (%) | (%) | Expect |
| gi\|400254\|sp\|P31422\| | METABOTROPIC | 879 | 864/879 | 874/879 | 0.0 |
| MGR3_RAT | GLUTAMATE | | (98%) | (99%) | |
| | RECEPTOR 3 | | | | |
| | PRECURSOR | | | | |
| gi\|6288800\|gb\|AAF06741.1\| | metabotropic | 879 | 877/879 | 878/879 | 0.0 |
| AF170701_1 | glutamate | | (99%) | (99%) | |
| | receptor 3 | | | | |
| | protein [Mus | | | | |
| | musculus] | | | | |
| gi\|11279202\|pir\|\|JC7160 | metabotropic | 879 | 875/879 | 876/879 | 0.0 |
| | glutamate | | (99%) | (99%) | |
| | receptor subtype | | | | |
| | 3 precursor - | | | | |
| | mouse | | | | |

The homology of these sequences is shown graphically in the ClustalW analysis shown in Table 6D.

[image]

[image]

[image]

Table 6E-F lists the domain description from DOMAIN analysis results against NOV6. This indicates that the NOV6 sequence has properties similar to those of other proteins known to contain this domain.

TABLE 6E

Domain Analysis of NOV6
gnl|Pfam|pfam01094, ANF_receptor, Receptor family ligand binding
region. This family includes extracellular ligand binding domains of a
wide range of receptors. This family also includes the bacterial amino
acid binding proteins of known structure.
(SEQ ID NO: 84)
CD-Length = 402 residues, 98.5% aligned
Score = 323 bits (827), Expect = 3e−89

| | | | |
|---|---|---|---|
| Query: | 62 | EDRGIQRLEAMLFAIDEINKDNYLLPGVKLGVHILDTCSRDTYALEQSLEFVRASLTKVD | 121 |
| | | \|\|\| \|\|\|\|\|\| \| \| \|\| \| \|\|\|\|+ \|\|+ \|\|\| \| \|\|\|\|\| \|\| \| \| \| | |
| Sbjct: | 6 | AVRGITRLEAMLGAFDRINADPALLPGLALGLAILDINSLRNVALEQSFTFVYGLLIKCD | 65 |
| Query: | 122 | EAEYMCPDGSYAIQENIPLLIAGVIGGSYSSVSIQVANLLRLFQIPQISYASTSAKLSDK | 181 |
| | | + \| \| \|+ + +\| \| \| \|\|\| \| \|\| \|\|\| \|\|+ +\|\|\|\| | |
| Sbjct: | 66 | CSSVRCAGGDLALTHGVAGVIGPSCSSSAIQV----ANLASLLNIPMISYGSTAPELSDK | 121 |
| Query: | 182 | SRYDYFARTVPPDFYQAKAMAEILRYFNWTYVSTVASEGDYGETGIEAFEQEARLRNICI | 241 |
| | | +\|\| \|+\|\|+\| \| +\| \|\| +\| ++\|\|\| \|\|\| \| \|+\| \|\|\| \| \|\|\| + \| \| \|\| | |
| Sbjct: | 122 | TRYPTFSRTIPSDAFQGLAMVDIFKHFNWNYVSVVYSDGTYGEEGCEAFIEALRARGGCI | 181 |
| Query: | 242 | ATAEKVGR--SNIRKSYDSVIRELLQKPNARVVVLFMRSDDSRELIAAASRVN--ASFTW | 297 |
| | | \| + \|+\| + +\| ++\|\|\| + \|\|\|\|\|+ + \|\|\|+ \|\| \|+ | |
| Sbjct: | 182 | ALSVKIGEFDRGDEEDFDKLLRELKR--RARVVVMCGHGETLRELLEAALRLGLTGEDYV | 239 |
| Query: | 298 | VASDGWGAQESIVKGSEHVAYGAITLELASHPVRQFDRYFQSLNPYNNHRNPWFRDFWEQ | 357 |
| | | \|\| + \| \| \|\|\| \|\|\|+ + +\| \|\| + \|\| \|\|\|\|\| +\|\|++ | |
| Sbjct: | 240 | FISDDLFNKSLPA---EPGAPGAI--ELANASMLRFAYYFVLVLTLNNPRNPWFLEFWKE | 294 |
| Query: | 358 | KFQCSLQNKRNHRQICDKHLAIDSSNYEQESKIMFVVNAVYAMAHALHKMQRTLCPNTT- | 416 |
| | | \| \|+\|\|+ \|\|\|\| \| \|\| +\|\|\| \|\|\|\|\| \| + | |
| Sbjct: | 295 | NFICALQDFLT------------LEPYEQEGKAGFVYDAVYLYAHALHNTTLALGGSWVD | 342 |
| Query: | 417 | --KLCDAMKILDGKKLYKDYLLKINFLAPFNPNKGADSIVKFDTYGDGMGRYNVFNFQHI | 474 |
| | | \|\| + + \| \| \| \|\| \|\| \| \| + + \|+ | |
| Sbjct: | 343 | GEKLVQHL-------------RNLTFEGVTGP-------VTFDENGDRDGDYVLLDTQNT | 382 |
| Query: | 475 | GGK-----YSYLKVGHWAE | 488 |
| | | +\| \|\| \| \| | |
| Sbjct: | 383 | ETGQLKVTGTYDGVGKWTE | 401 |

TABLE 6F

Domain Analysis of NOV6
gnl/Pfam/pfam00003, 7tm_3, 7 transmembrane receptor (metabotropic E
family).
(SEQ ID NO: 85)
CD-Length = 256 residues, 100.0% aligned
Score = 323 bits (827), Expect = 3e−89

| | | | |
|---|---|---|---|
| Query: | 576 | WAIGPVTIACLGFMCTCIVITVFIKHNNTPLVKASGRELCYILLFGVSLSYCMTFFFIAK | 635 |
| | | \| \| +\| \|\| + \| \|+ \|\|+\|\| +\|\|+\|\|\|\| \|\|\| \|+\|\| \|+ \| \| +\| \|\| \| | |
| Sbjct: | 1 | LGIVLVALAVLGIVLTLFVLVVFVKHRDTPIVKASNRELSYLLLIGLILCYLCSFLFIGK | 60 |
| Query: | 636 | PSPVICALRRLGLGTSFAICYSALLTKTNCIARIFDGVKNGAQRPKFISPSSQVFICLGL | 695 |
| | | \|\| \| \|\|\|+ \| \| +\|\|\|\|\|\| \|\|\| + \|\|\| \| \|+ +\|\|\|\|\|\| +\|\| \| \| \| | |
| Sbjct: | 61 | PSETSCILRRILFGLGFTLCYSALLAKTNRVLRIFRAKKPGSGKPKFISPWAQVLIVLIL | 120 |
| Query: | 696 | ILVQIVMVSVWLILETPGTRRYTLPEKRETVILKCNVKDS-SMLISLTYDVVLVILCTVY | 754 |
| | | +\|+\|+++ +\|\|++\| \| \|\| + +\|\|+\|\| + ++ \| \|\| +\| +\|\|\| | |
| Sbjct: | 121 | VLIQVIICVIWLVVEPPRPTIDIYSEKEK-IILECNKGSMVAFVVVLGYDGLLAVLCTFL | 179 |
| Query: | 755 | AFKTRKCPENFNEAKPIGFTMYTTCIIWLAFLPIFYVTSSDYRVQTTTMCISVSLSGFVV | 814 |
| | | \|\| \|\| \|\|\|\|\|\|\|\|\|\|\|\|+\| \| \|\|+\|+\|\|+\|\|+ \|+ +\|\| \|+ \| \|+ | |
| Sbjct: | 180 | AFLTRNLPENFNEAKFIGFSMLTFCIVWVAFIPIYL--STPGKVQVAVEIFSILASSTVL | 237 |
| Query: | 815 | LGCLFAPKVHIVLFQPQKN | 833 |
| | | \|\|\|\|\| \|\| +\|+\|\|+\|+\|\| | |
| Sbjct: | 238 | LGCLFVPKCYIILFRPEKN | 256 |

G-protein-coupled receptors (GPCRs) constitute a vast protein family that encompasses a wide range of functions (including various autocrine, paracrine and endocrine processes). They show considerable diversity at the sequence level, on the basis of which they can be separated into distinct groups. The term clan is used to describe the GPCRs, as they embrace a group of families for which there are indications of evolutionary relationship, but between which there is no statistically significant similarity in sequence. The currently known clan members include the rhodopsin-like GPCRs, the secretin-like GPCRs, the cAMP receptors, the fungal mating pheromone receptors, and the metabotropic glutamate receptor family.

The metabotropic glutamate receptors are functionally and pharmacologically distinct from the ionotropic glutamate receptors. They are coupled to G-proteins and stimulate the inositol phosphate/Ca²⁺ intracellular signalling pathway. The amino acid sequences of the receptors contain high proportions of hydrophobic residues grouped into 7 domains, in a manner reminiscent of the rhodopsins and other receptors believed to interact with G-proteins. However, while a similar 3D framework has been proposed to account for this, there is no significant sequence identity between these and receptors of the rhodopsin-type family: the metabotropic glutamate receptors thus bear their own distinctive ‘7TM’ signature. This 7TM signature is also shared by the calcium-sensing receptors, and GABA (gamma-amino-butyric acid) type B (GABA(B)) receptors.

At least eight sub-types of metabotropic receptor (MGR1-8) have been identified in cloning studies. The sub-types differ in their agonist pharmacology and signal transduction pathways.

The mGluR3 gene consists of six exons and spans over 95 kb. Exon 1 and its preceding putative promoter are located distantly from the following protein-coding region. In the mGluR family, mGluR3 and mGluR5 are both expressed in neuronal and glial cells and are upregulated during the early postnatal period. They are, however, coupled to two distinct signaling cascades and have been shown to exert opposite influences on some functions of cultured astrocytes. In cultured astrocytes, mGluR3 and mGluRS mRNA levels were significantly increased by exposure to epidermal growth factor (EGF), basic fibroblast growth factor (bFGF), or transforming growth factor-alpha; and EGF was more efficacious than bFGF in producing this increase. Hence, mGluR3 and mGluR5 mRNAs are concertedly upregulated in cultured astrocytes by specific growth factors. This finding suggests that the two mGluR subtypes may play an important role in maintaining the proper balance of astrocyte functions via two distinct signal transduction mechanisms.

Glutamate receptors are divided into 2 distinct classes: ionotropic glutamate receptors (iGluRs) and metabotropic glutamate receptors (mGluRs). The iGluRs consist of N-methyl-D-aspartate (NMDA) receptors and non-NMDA receptors. Non-NMDA receptors are further subdivided into 2 groups: alpha-amino-3-hydroxy-5-methyl-4-isoxazolepropionic acid receptors and kainate receptors. The mGluRs are divided into 3 groups according to agonist selectivity, coupling to different effector systems, and sequence homology. Group I includes mGluR1 and mGluR5, which are coupled to inositol phospholipid metabolism. Group II, which includes mGluR2 and mGluR3, and group III, which includes mGluR4, mGluR6, mGluR7, and mGluR8, are negatively coupled to adenylate cyclase activity. Each mGluR possesses a large extracellular domain. Okamoto et al. (1998) expressed mGlur1-alpha (mGluR1A) in insect cells on a baculovirus system. They isolated a soluble mGluR that encodes only the extracellular domain and retains a ligand binding characteristic similar to that of the full-length receptor. Their observations demonstrated that a ligand binding event in mGluRs can be dissociated from the membrane domain.

Smitt et al. (2000) demonstrated that autoantibody against mGluR1A was responsible for severe paraneoplastic cerebellar ataxia in 2 patients. The disorder developed in both patients while they were in remission from Hodgkin disease. One, a teenager, had been in remission for 2 years when truncal ataxia, intention tremor, and gait ataxia developed. This patient improved clinically with loss of cells in the cerebrospinal fluid when treated with plasma exchanges, oral prednisone, and 2 courses of intravenous immune globulin. The second patient reported by Smitt et al. (2000) was in her late forties and, in addition to successfully treated Hodgkin disease, had polycystic kidney disease requiring hemodialysis for many years. Therapy was less successful in this patient, possibly because of delay in initiation.

The disclosed NOV6 nucleic acid of the invention encoding a Metabotropic Glutamate Receptor-like protein includes the nucleic acid whose sequence is provided in Table 6A or a fragment thereof. The invention also includes a mutant or variant nucleic acid any of whose bases may be changed from the corresponding base shown in Table 6A while still encoding a protein that maintains its Metabotropic Glutamate Receptor-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 10% percent of the bases may be so changed.

The disclosed NOV6 protein of the invention includes the Metabotropic Glutamate Receptor-like protein whose sequence is provided in Table 6B. The invention also includes a mutant or variant protein any of whose residues may be changed from the corresponding residue shown in Table 6B while still encoding a protein that maintains its Metabotropic Glutamate Receptor-like activities and physiological functions, or a functional fragment thereof. In the mutant or variant protein, up to about 10% percent of the residues may be so changed.

The above defined information for this invention suggests that these Metabotropic Glutamate Receptor-like proteins (NOV6) may function as a member of a “Metabotropic Glutamate Receptor family”. Therefore, the NOV6 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The nucleic acids and proteins of NOV6 are useful in potential therapeutic applications implicated in immune disorders and airway pathologies such as epileptic seizures and other neurological disorders, Hodgkin disease, polycystic kidney disease, mental depression, Adenocarcinoma, Smith-Lemli-Opitz syndrome, Retinitis pigmentosa, and/or other pathologies and disorders For example, a cDNA encoding NOV6 may be useful in gene therapy, and NOV6 may be useful when administered to a subject in need thereof. By way of nonlimiting example, NOV6 will have efficacy for treatment of patients suffering from epileptic seizures and other neurological disorders, Hodgkin disease, polycystic kidney disease, mental depression, Adenocarcinoma, Smith-Lemli-Opitz syndrome, Retinitis pigmentosa. The novel NOV6 nucleic acid encoding NOV6 protein, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

NOV6 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV6 protein have multiple hydrophilic regions, each of which can be used as an immunogen. In one embodiment, contemplated NOV6 epitope is from about amino acids 20 to 30. In other embodiments, NOV6 epitope is from about amino acids 50 to 70, from about amino acids 100 to 140, from about amino acids 180 to 200, from about amino acids 210 to 280, from about amino acids 310 to 400, from about amino acids 450 to 510, from about amino acids 520 to 560, from about amino acids 600 to 610, from about amino acids 660 to 680, from about amino acids 700 to 720, from about amino acids 750 to 770, or from about amino acids 800 to 850. This novel protein also has value in development of powerful assay system for functional analysis of various human disorders, which will help in understanding of pathology of the disease and development of new drug targets for various disorders.

NOV7

NOV7 includes three novel PV-1-like proteins disclosed below. The disclosed proteins have been named NOV7a, NOV7b, and NOV7c.

NOV7a

A disclosed NOV7a nucleic acid of 1366 nucleotides (also referred to sggc_draft_ba560a15_{—}20000723_da1) encoding a novel PV-1-like receptor protein is shown in Table 7A. An open reading frame was identified beginning with an ATG initiation codon at nucleotides 1-3 and ending with a TGA codon at nucleotides 1327-1329. In Table 7A, the 3′ untranslated region is underlined and the start and stop codons are in bold letters.

TABLE 7A
NOV7a Nucleotide Sequence (SEQ ID NO: 18)

ATGGGTCTGGCCATGGAGCACGGAGGGTCCTACGCTCGGGCGGGGGGCAGCTCTCGGGGCTGCTGGTATTA
CCTGCGCTACTTCTTCCTCTTCGTCTCCCTCATCCAATTCCTCATCATCCTGGGGCTCGTGCTCTTCATGG
TCTATGGCAACGTGCACGTGAGCACAGAGTCCAACCTGCAGGCCACCGAGCGCCGAGCCGAGGGCCTATAC
AGTCAGCTCCTAGGGCTCACGGCCTCCCAGTCCAACTTGACCAAGGAGCTCAACTTCACCACCCGCGCCAA
GGATGCCATCATGCAGATGTGGCTGAATGCTCGCCGCGACCTGGACCGCATCAATGCCAGCTTCCGCCAGT
GCCAGGGTGACCGGGTAATCTACACGAACAATCAGAGGTACATGGCTGCCATCATCTTGAGTGAGAAGCAA
TGCAGAGATCAATTCAAGGACATGAACAAGAGCTGCGATGCCTTGCTCTTCATGCTGAATCAGAAGGTGAA
GACGCTGGAGGTGGAGATAGCCAAGGAGAAGACCATTTGCACTAAGGATAAGGAAAGCGTGCTGCTGAACA
AACGCGTGGCGGAGGAACAGCTGGTTGAATGCGTGAAAACCCGGGAGCTGCAGCACCAAGAGCGCCAGCTG
GCCAAGGAGCAACTGCAAAAGGTGCAAGCCCTCTGCCTGCCCCTGGACAAGGACAAGTTTGAGATGGACCT
TCGTAACCTGTGGAGGGACTCCATTATCCCACGCAGCCTGGACAACCTGGGTTACAACCTCTACCATCCCC
TGGGCTCGGAATTGGCCTCCATCCGCAGAGCCTGCGACCACATGCCCAGCCTCATGAGCTCCAAGGTGGAA
GGTCAGTGCCGGAGCCTCCGGGCGGATATCGAACGCGTGGCCCGCGAGAACTCAGACCTCCAACGCCAGAA
GCTGGAAGCCCAGCAGGGCCTGCGGGCCAGTCAGGAGGCGAAACAGAAGGTGGAGAAGGAGGCTCAGGCCC
GGGAGGCCAAGCTCCAAGCTGAATGCTCCCGGCAGACCCAGCTAGCGCTGGAGGAGAAGGCGGTGCTGCGG
AAGGAACGAGACAACCTGGCCAAGGAGCTGGAAGAGAAGAAGAGGGAGGCGGAGCAGCTCAGGATGGAGCT
GGCCATCAGAAACTCAGCCCTGGACACCTGCATCAAGACCAAGTCGCAGCCGATGATGCCAGTGTCAAGGC
CCATGGGCCCTGTCCCCAACCCCCAGCCCATCGACCCAGCTAGCCTGGAGGAGTTCAAGAGGAAGATCCTG
GAGTCCCAGAGGCCCCCTGCAGGCATCCCTGTAGCCCCATCCAGTGGCTGAGGAGGCTCCGGCACTGACCT
AAGGGCGAATCCCAGCA

The disclosed NOV7a nucleic acid sequence, localized to chromosome 19, has 945 of 1345 bases (70%) identical to a 1968 bp PV-1 mRNA from Rattus norvegicus (GENBANK-ID: AF15483|acc:AF154831) (E=1.1e⁻¹²¹).

A disclosed NOV7a polypeptide (SEQ ID NO:19) encoded by SEQ ID NO:18 is 442 amino acid residues and is presented using the one-letter amino acid code in Table 7B. Signal P, Psort and/or Hydropathy results predict that NOV7a has a signal peptide and is likely to be localized in the plasma membrane with a certainty of 0.7900. In other embodiments, NOV7a is also likely to be localized to the nucleus with a certainty of 0.6000, to the microbody (peroxisome) with a certainty of 0.3000, or the Golgi body with a certainty of 0.3000. The most likely cleavage site for a NOV7a peptide is between amino acids 50 and 51, at YVG-NV.

SNP data for NOV7a can be found below in Example 3.

TABLE 7B
Encoded NOV7a protein sequence (SEQ ID NO: 19).

MGLAMEHGGSYARAGGSSRGCWYYLRYFFLFVSLIQFLIILGLVLFMVYGNVHVSTESNLQATE
RRAEGLYSQLLGLTASQSNLTKELNFTTRAKDAIMQMWLNARRDLDRINASFRQCQGDRVIYTN
NQRYMAAIILSEKQCRDQFKDMNKSCDALLFMLNQKVKTLEVEIAKEKTICTKDKESVLLNKRV
AEEQLVECVKTRELQHQERQLAKEQLQKVQALCLPLDKDKFEMDLRNLWRDSIIPRSLDNLGYN
LYHPLGSELASIRRACDHMPSLMSSKVEGQCRSLRADIERVARENSDLQRQKLEAQQGLRASQE
AKQKVEKEAQAREAKLQAECSRQTQLALEEKAVLRKERDNLAKELEEKKREAEQLRMELAIRNS
ALDTCIKTKSQPMMPVSRPMGPVPNPQPIDPASLEEFKRKILESQRPPAGIPVAPSSG

The disclosed NOV7a amino acid sequence has 266 of 442 amino-acid residues (60%) identical to, and 347 of 442 amino acid residues (78%) similar to, the 438 amino acid residue PV-1 protein from Rattus norvegicus (SPTREMBL-ACC:Q9WV78) ( 1.6e−¹⁴²), and 439 of 442 amino acid residues (99%) identical to, and 439 of 442 amino acid residues (99%) similar 20 to, the 479 amino acid residue Human ORFX ORF1918 polypeptide sequence (patp:AAB42154 ) (3.2e⁻²²⁹).

TaqMan data for NOV7a can be found below in Example 2.

NOV7b

A disclosed NOV7b nucleic acid of 1421 nucleotides (also referred to 2847264.0.32) encoding a novel PV-1-like receptor protein is shown in Table 7C. An open reading frame was identified beginning with an ATG initiation codon at nucleotides 55-57 and ending with a TGA codon at nucleotides 1381-1383. In Table 7C, the 5′ and 3′ untranslated regions are underlines and the start and stop codons are in bold letters.

TABLE 7C
NOV7b Nucleotide Sequence (SEQ ID NO: 20)

GAATTCTAGGTGGTGGTGAGCAGGGACGGTGCACCGGACGGCGGGATCGAGCAAATGGGTCTGGCCATGGA
GTACGGAGGGTCCTACGCTCGGGCGGGGGGCAGCTCTCGGGGCTGCTGGTATTACCTGCGCTACTTCTTCC
TCTTCGTCTCCCTCATCCAATTCCTCATCATCCTGGGGCTCGTGCTCTTCATGGTCTATGGCGACGTGCAC
GTGAGCACAGAGTCCAACCTGCAGGCCACCGAGCGCCGAGCCGAGGGCCTATACAGTCAGCTCCTAGGGCT
CACGGCCTCCCAGTCCAACTTGACCAAGGAGCTCAACTTCACCACCCGCGCCAAGGATGCCATCATGCAGA
TGTGGCTGAATGCTCGCCGCGACCTGGACCGCATCAATGCCAGCTTCCGCCAGTGCCAGGGTGACCGGGTC
ATCTACACGAACAATCAGAGGTACATGGCTGCCATCATCTTGAGTGAGAAGCAATGCAGAGATCAATTCAA
GGACATGAACAAGAGCTGCGATGCCTTGCTCTTCATGCTGAATCAGAAGGTGAAGACGCTGGAGGTGGAGA
TAGCCAAGGAGAAGACCATTTGCACTAAGGATAAGGAAAGCGTGCTGCTGAACAAACGCGTGGCGGAGGAA
CAGCTGGTTGAATGCGTGAAAACCCGGGAGCTGCAGCACCAAGAGCGCCAGCTGGCCAAGGAGCAACTGCA
AAAGGTGCAAGCCCTCTGCCTGCCCCTGGACAAGGACAAGTTTGAGATGGACCTTCGTAACCTGTGGAGGG
ACTCCATTATCCCACGCAGCCTGGACAACCTGGGTTACAACCTCTACCATCCCCTGGGCTCGGAATTGGCC
TCCATCCGCAGAGCCTGCGACCACATGCCCAGCCTCATGAGCTCCAAGGTGGAGGAGCTGGCCCGGAGCCT
CCGGGCGGATATCGAACGCGTGGCCCGCGAGAACTCAGACCTCCAACGCCAGAAGCTGGAAGCCCAGCAGG
GCCTGCGGGCCAGTCAGGAGGCGAAACAGAAGGTGGAGAAGGAGGCTCAGGCCCGGGAGGCCAAGCTCCAA
GCTGAATGCTCCCGGCAGACCCAGCTAGCGCTGGAGGAGAAGGCGGTGCTGCGGAAGGAACGAGACAACCT
GGCCAAGGAGCTGGAAGAGAAGAAGAGGGAGGCGGAGCAGCTCAGGATGGAGCTGGCCATCAGAAACTCAG
CCCTGGACACCTGCATCAAGACCAAGTCGCAGCCGATGATGCCAGTGTCAAGGCCCATGGGCCCCTGTCCC
AACCCCCAGCCCATCGACCCAGCTAGCCTGGAGGAGTTCAAGAGGAAGATCCTGGAGTCCCAGAGGCCCCC
TGCAGGCATCCCTGTAGCCCCATCCAGTGGCTGAGGAGGCTCCAGGCCTGAGGACCAAGGGATGGCCCGAC
T

The disclosed NOV7b nucleic acid sequence, localized to chromosome 19, has 969 of 1383 bases (70%) identical to a PV-I mRNA from Rattus norvegicus (GENBANK-ID: AF154831 ) (E=2.5e⁻¹²³).

A disclosed NOV7b polypeptide (SEQ ID NO:21) encoded by SEQ ID NO:20 is 442 amino acid residues and is presented using the one-letter amino acid code in Table 7D. Signal P, Psort and/or Hydropathy results predict that NOV7b has a signal peptide and is likely to be localized in the plasma membrane with a certainty of 0.7900. In other embodiments, NOV7b is also likely to be localized to the nucleus with a certainty of 0.6000, to the microbody (peroxisome) with a certainty of 0.3000, or the Golgi body with a certainty of 0.3000. The most likely cleavage site for a NOV7b peptide is between amino acids 50 and 51, at: YVG-NV.

TABLE 7D
Encoded NOV7b protein sequence (SEQ ID NO: 21).

MGLAMEYGGSYARAGGSSRGCWYYLRYFFLFVSLIQFLIILGLVLFMVYGDVHVSTESNLQATERRAEGLY
SQLLGLTASQSNLTKELNFTTRAKDAIMQMWLNARRDLDRINASFRQCQGDRVIYTNNQRYMAAIILSEKQ
CRDQFKDMNKSCDALLFMLNQKVKTLEVEIAKEKTICTKDKESVLLNKRVAEEQLVECVKTRELQHQERQL
AKEQLQKVQALCLPKDKDKFEMDLRNLWRDSIIPRSLDNLGYNLYHPLGSELASIRRACDHMPSLMSSKVE
ELARSLRADIERVARENSDLQRQKLEAQQGLRASQEAKQKVEKEAQAREAKLQAECSRQTQLALEEKAVLR
KERDNLAKELEEKKREAEQLRMELAIRNSALDTCIKTKSQPMMPVSRPMGPVPNPQPIDPASLEEFKRKIL
ESQRPPAGIPVAPSSG

The disclosed NOV7b amino acid sequence has 268 of 442 amino acid residues (60%) identical to, and 350 of 442 amino acid residues (79%) similar to the 438 amino acid residue PV-1 protein from Rattus norvegicus (SPTREMBL-ACC:Q9WV78), and 454of 457 amino acid residues (99%) identical to, and 457 of 457 amino acid residues (100%) similar to the 479 amino acid residue Human ORFX ORF1918 polypeptide sequence (patp:AAB42154) (E=1.3e⁻²³⁷).

NOV7b is expressed in at least the following tissues: lymph node, bone marrow, spleen, mammary gland, thyroid, stomach, fetal kidney, heart, fetal liver. In addition, the sequence is predicted to be expressed in lung because of the expression pattern of (GENBANK-ID: Q9WV78) a closely related PV-1 homolog in species Rattus norvegicus. It has also been reported to be expressed in muscle and brain (J Cell Biol 1999 Jun. 14; 145(6): 1189-98). Endothelium of the fenestrated peritubular capillaries of the kidney and those of the intestinal villi, pancreas, and adrenals have also been shown to express PV-1 (Proc Natl Acad Sci USA 1999 Nov. 9;96(23):13203-7) TaqMan data for NOV7b can be found below in Example 2.

NOV7c

A disclosed NOV7c nucleic acid of 2024 nucleotides (also referred to CG51878-03) encoding a novel PV-1-like receptor protein is shown in Table 7E. An open reading frame was identified beginning with an ATG initiation codon at nucleotides 1-3 and ending with a TGA codon at nucleotides 1327-1329. In Table 7E, the 3′ untranslated region is underlined and the start and stop codons are in bold letters.

TABLE 7A
NOV7c Nucleotide Sequence (SEQ ID NO: 22)

ATGGGTCTGGCCATGGAGCACGGAGGGTCCTACGCTCGGGCGGGGGGCAGCTCTCGGGGCTGCTGGTATTA
CCTGCGCTACTTCTTCCTCTTCGTCTCCCTCATCCAATTCCTCATCATCCTGGGGCTCGTGCTCTTCATGG
TCTATGGCAACGTGCACGTGAGCACAGAGTCCAACCTGCAGGCCACCGAGCGCCGAGCCGAGGGCCTATAC
AGTCAGCTCCTAGGGCTCACGGCCTCCCAGTCCAACTTGACCAAGGAGCTCAACTTCACCACCCGCGCCAA
GGATGCCATCATGCAGATGTGGCTGAATGCTCGTCGCGACCTGGACCGCATCAATGCCAGCTTCCGCCAGT
GCCAGGGTGACCCGGTCATCTACACGAACAATCAGAGGTACATGGCTGCCATCATCTTGAGTGAGAAGCAA
TGCAGAGATCAATTCAAGGACATGAACAAGAGCTGCGATGCCTTGCTCTTCATGCTGAATCAGAAGGTGAA
GACGCTGGAGGTGGAGATAGCCAAGGAGAAGACCATTTGCACTAAGGATAAGGAAAGCGTGCTGCTGAACA
AACGCGTGGCGGAGGAACAGCTGGTTGAATGCGTGAAAACCCGGGAGCTGCAGCACCAAGAGCGCCAGCTG
GCCAAGGAGCAACTGCAAAGGGTGCAAGCCCTCTGCCTGCCCCTGGACAACGACAAGTTTGAGATGGACCT
TCGTAACCTGTGGAGGGACTCCATTATCCCACGCAGCCTGGACAACCTGGGTTACAACCTCTACCATCCCC
TGGGCTCGGAATTGGCCTCCATCCGCAGAGCCTGCGACCACATGCCCAGCCTCGTGAGCTCCAACGTGGAG
GAGCTGGCCCGGAGCCTCCGGGCGGATATCGAACGCGTGGCCCGCGAGAACTCAGACCTCCAACGCCAGAA
GCTCGAAGCCCAGCAGGGCCTGCGGGCCACTCAGGAGGCGAAACACAAGGTGGAGAAGGAGGCTCAGGCCC
GGGAGGCCAAGCTCCAAGCTGAATGCTCCCGGCAGACCCAGCTAGCGCTGGAGGAGAAGGCGGTGCTGCGG
AAGGAACGAGACAACCTGGCCAAGGAGCTGGAAGAGAAGAAGAGGGAGGCGGACCAGCTCAGGATGGAGCT
GGCCATCAGAAACTCAGCCCTGGACACCTGCATCAAGACCAAGTCGCAGCCGATGATGCCAGTGTCAAGGC
CCATGGGCCCTGTCCCCAACCCCCAGCCCATCGACCCAGCTAGCCTGGAGGAGTTCAAGAGGAAGATCCTG
GAGTCCCAGAGGCCCCCTGCAGGCATCCCTGTAGCCCCATCCAGTGGCTGAGGAGGCTCCAGGCCTGAGGA
CCAAGGGATGGCCCGACTCGGCGGTTTGCGGAGGATGCAGGGATATGCTCACAGCGCCCGACACAACCCCC
TCCCGCCGCCCCCAACCACCCAGGGCCACCATCAGACAACTCCCTGCATGCAAACCCCTAGTACCCTCTCA
CACCCGCACCCGCGCCTCATGATCCCTCACCCAGAGCACACGGCCGCGGAGATGACGTCACGCAAGCAACG
GCGCTGACGTCACATATCACCGTGGTGATGGCGTCACGTCGCCATGTAGACGTCACGAAGAGATATAGCGA
TGGCGTCGTGCAGATGCAGCACGTCGCACACAGACATGGGGAACTTGGCATGACGTCACACCGAGATGCAG
CAACGACGTCACGGGCCATGTCGACGTCACACATATTAATGTCACACAGACGCGGCGATGGCATCACACAG
ACGGTGATGATGTCACACACAGACACAGTGACAACACACACCATGACAACGACACCTATAGATATGGCACC
AACATCACATGCACGCATGCCCTTTCACACACACTTTCTACCCAATTCTCACCTAGTGTCACGTTCCCCCG
ACCCTGGCACACGGGCCAAGGTACCCACAGGATCCCATCCCCTCCCGCACAGCCCTGGGCCCCAGCACCTC
CCCTCCTCCAGCCTCCTGGCCTCCCGGTAGTACACG

The disclosed NOV7c nucleic acid sequence, localized to chromosome 19p13, has 2009 of 2015 bases (99%) identical to a gb:GENBANK-ID:AF326591|acc:AF326591.1 mRNA from Homo sapiens (Homo sapiens fenestrated-endothelial linked structure protein (FELS) mRNA, complete cds) (E=0.0).

A disclosed NOV7c polypeptide (SEQ ID NO:23) encoded by SEQ ID NO:22 is 442 amino acid residues and is presented using the one-letter amino acid code in Table 7F. Signal P, Psort and/or Hydropathy results predict that NOV7c has a signal peptide and is likely to be localized in the plasma membrane with a certainty of 0.7900. In other embodiments, NOV7c is also likely to be localized to the nucleus with a certainty of 0.6000, to the microbody (peroxisome) with a certainty of 0.3000, or the Golgi body with a certainty of 0.3000. The most likely cleavage site for a NOV7c peptide is between amino acids 50 and 51, at: YVG-NV.

TABLE 7F
Encoded NOV7c protein sequence (SEQ ID NO: 23).

MGLAMEHGGSYARAGGSSRGCWYYLRYFFLFVSLIQFLIILGLVLFMVYGNVHVSTESNLQATERRAEGLY
SQLLGLTASQSNLTKELNFTTRAKDAIMQMWLNARRDLDRINASFRQCQGDRVIYTNNQRYMAAIILSEKQ
CRDQFKDMNKSCDALLFMLNQKVKTLEVEIAKEKTICTKDKESVLLNKRVAEEQLVECVKTRELQHQERQL
AKEQLQRVQALCLPLDKDKFEMDLRNLWRDSIIPRSLDNLGYNLYHPLGSELASIRRACDHMPSLVSSKVE
ELARSLRADIERVARENSDLQRQKLEAQQGLRASQEAKQKVEKEAQAREAKLQAECSRQTQLALEEKAVLR
KERDNLAKELEEKKREAEQLRMELAIRNSALDTCIKTKSQPMMPVSRPMGPVPNPQPIDPASLEEFKRKIL
ESQRPPAGIPVAPSSG

The disclosed NOV7c amino acid sequence has 440 of 442 amino acid residues (99%) identical to, and 442 of 442 amino acid residues (100%) similar to, the 442 amino acid residue ptnr:SPTREMBL-ACC:Q9BX97 protein from Homo sapiens (Human) (PV1 PROTEIN) (E=6.9e⁻²³¹).

NOV7c is expressed in at least the following tissues: Heart, Adrenal Gland/Suprarenal gland, Thyroid, Salivary Glands, Liver, Bone Marrow, Spleen, Lymph Node, Mammary gland/Breast, Placenta, Prostate, Lung, Kidney, Pancreas, Bone Marrow, and Small Intestine. Expression information was derived from the tissue sources of the sequences that were included in the derivation of the sequence of CuraGen Acc. No. CG51878-03. The sequence is predicted to be expressed in the following tissues because of the expression pattern of (GENBANK-ID: gb:GENBANK-ID:AF326591|acc:AF326591.1) a closely related Homo sapiens fenestrated-endothelial linked structure protein (FELS) mRNA.

The NOV7a, 7c and 7c are very closely homologous as is shown in the alignment in Table 7G.

[image]

[image]

Homologies to any of the above NOV7 proteins will be shared by the other two NOV7 proteins insofar as they are homologous to each other as shown above. Any reference to NOV7 is assumed to refer to all three of the NOV7 proteins in general, unless otherwise noted.

NOV7a also has homology to the amino acid sequence shown in the BLASTP data listed in Table 7H.

TABLE 7H
BLAST results for NOV7a

| Gene Index/ | | Length | Identity | Positives | |
|---|---|---|---|---|---|
| Identifier | Protein/Organism | (aa) | (%) | (%) | Expect |
| gi\|12963353\|gb\|AAK11226.1\| | fenestrated- | 442 | 438/442 | 438/442 | 0.0 |
| | endothelial | | (99%) | (99%) | |
| | linked structure | | | | |
| | protein [Homo | | | | |
| | sapiens] | | | | |
| gi\|13775238\|ref\|NP_112600.1\| | fenestrated- | 442 | 439/442 | 439/442 | 0.0 |
| | endothelial | | (99%) | (99%) | |
| | linked structure | | | | |
| | protein; PV-1 | | | | |
| | protein [Homo | | | | |
| | sapiens]) | | | | |
| gi\|9910520\|ref\|NP_064471.1\| | PV-1 [Rattus | 438 | 266/442 | 347/442 | e−131 |
| | norvegicus] | | (60%) | (78%) | |
| gi\|14161394\|gb\|AAK54730.1\| | MECA32 [Mus | 438 | 270/442 | 348/442 | e−125 |
| AF369900_1 | musculus] | | (61%) | (78%) | |
| gi\|14161698\|ref\|NP_115774.1\| | plasmalemma | 438 | 269/442 | 346/442 | e−125 |
| | vesicle | | (60%) | (77%) | |
| | associated | | | | |
| | protein [Mus | | | | |
| | musculus] | | | | |

The homology of these sequences is shown graphically in the ClustalW analysis shown in Table 7I.

[image]

[image]

[image]

PV-1 is a novel endothelial protein shown by immunocytochemical tests to be specifically associated with the stomatal diaphragms of caveolae in lung endothelium (Stan R V, et.al.; Proc Natl Acad Sci USA 1999 Nov. 9;96(23):13203-7). Although the highest expression levels of both mRNA and protein are in the lung, PV-1 also has been found to be expressed in other organs. Using a specific antibody to the extracellular domain of PV-1, the survey on the presence of this protein at light and electron microscope level has been extended in several rat organs. It has been shown by immunofluorescence the antibody recognizes with high specificity the endothelium of the fenestrated peritubular capillaries of the kidney and those of the intestinal villi, pancreas, and adrenals. By immunolocalization at electron microscope level, the antibody recognizes specifically the diaphragms of the fenestrae and the stomatal diaphragms of caveolae and transendothelial channels in the endothelia of these vascular beds. No signal was detected in the continuous endothelium of the heart, skeletal muscle, intestinal muscularis, or brain capillaries or the nondiaphragmed fenestrated endothelium of kidneyglomeruli. Taken together, the findings define the only antigen to be localized thus far in fenestral diaphragms. They also show that the stomatal diaphragms of caveolae and transendothelial channels and the fenestral diaphragms might be biochemically related, in addition to being morphologically similar structures.

By using an immunoisolation procedure (Stan, R.-V., W. G. Roberts, K. Ihida, D. Predescu, L., Saucan, L. Ghitescu, and G. E. Palade. 1997. Mol. Biol. Cell. 8:595-605) developed in our laboratory, a caveolar subfraction from rat lung endothelium has been isolated and the proteins of this subfraction have been partially characterized which include an apparently caveolae-specific glycoprotein is proposed to be called PV-1 (formerly known as gp68). The isolation and partial sequencing of PV-1, combined with the cloning of the full length PV-l cDNA led to the following conclusions: (a) PV-1 is a novel single span type II integral membrane protein (438 amino acids long) which forms homodimers in situ; (b) the transmembrane domain of PV-1 is near the NH2 terminus defining a short cytoplasmic endodomain and a large COOH-terminal ectodomain exposed to the blood plasma; (c) PV-1 is N-glycosylated and its glycan antennae bear terminal nonreducing galactosyl residues in alpha1-3 linkage. PV-1 is expressed mostly in the lung but both the messenger RNA and the protein can be detected at lower levels also in kidney, spleen, liver, heart, muscle, and brain. No signal could be detected in testis and two lower molecular weight forms were detected in brain. Immunocytochemical studies carried out by immunodiffusion on rat lung with an anti-PV-1 polyclonal antibody directed against a COOH-terminal epitope reveal a specific localization of PV-1 to the stomatal diaphragms of rat lung endothelial caveolae and confirm the extracellular orientation of the PV-1 COOH terminus (Stan, R. V. J. Cell. Biol. 1999, Jun. 14; 145(6): 189-98).

Immunohistochemistry revealed initial expression of the stage-specific glycoprotein, GP68, in various mesenchymal tissue substructures of mouse embryos (Morita T, et.al.; Okajimas Folia Anat Jpn October 1998;75(4):185-95). During the 11-15th days of gestation, GP68 was localized in the primitive meninges, chondroblasts and perichondrium of pre-cartilaginous vertebral bodies and ribs, connective tissue cells of the dermis, the epicardium and endocardium of the heart, the epimysium and perimysium of skeleton musclature, and the basement membranes of splanchnic organs. Double staining for laminin expression indicated coincidental expression in identical tissue substructures. However, laminin was expressed in days 10-18 embryos and the neonate. Therefore, GP68 is coincidentally expressed with laminin in mesenchymal tissues between the 11th and 15th day of gestation, and may play a role as a laminin-associated protein. In the light of these results, a hypothesis concerning the relationship between these two proteins and the mechanisms of non-integrin laminin-associated proteins during normal embryogenesis is discussed further.

The microvascular endothelium is organized as a highly differentiated squamous epithelium whose main function is to mediate the exchanges of water, macromolecules, and small solutes between the blood plasma and the interstitial fluid. The endothelial structures implicated so far in the transendothelial transport are the caveolae, transendothelial channels, intercellular junctions, and the fenestrae. Caveolae are flask-shaped or spherical plasma membrane invaginations and associated vesicles of 70-nm average outer diameter that can occur singly or in chains or clusters. In invaginated form, their membranes is in continuity layer by layer with the plasmalemma proper, and, in some microvascular beds (e.g., the continuous endothelium of the lung and the fenestrated and sinusoidal endothelia), their introits or necks are provided with a stomatal diaphragm.

The transendothelial channels are channels of 60-70-nm diameter that run across the endothelial cell. They seem to be formed by the fusion of either one caveola with both luminal and abluminal aspects of the plasmalemmna or by chains of usually two to four caveolae. These channels are provided with two diaphragms (one luminal and one abluminal) only in fenestrated endothelia and not in their continuous counterparts.

The diaphragmed fenestrae are characteristic structural elements of all fenestrated endothelia (e.g., kidney peritubular capillaries and ascending vasa recta, capillaries of intestinal villi, pancreas, adrenal cortex, endocrine glands, and choriocapillaries of the brain and eye). They are round openings or windows cutting through the endothelial cell, have a constant diameter of 63-68 nm, and occur only in the attenuated parts of the cell, in clusters referred to as “sieve plates”. In en face electron microscopic images, the fenestrae appear circular, but several studies have shown that they have an 8-fold symmetry. The rim of the fenestra (where the abluminal plasmalemma is continuing the luminal plasmalemma) is the anchoring line for the fenestral diaphragm. In normal sections, the diaphragm appears as a very thin (5-6 nm) single-layer barrier provided with a central density or knob. Deep-etch rapid-freeze techniques have revealed the structure of the diaphragm to be composed of radial fibrils (7-nm diameter) starting at the rim and interweaving in a central mesh (the equivalent of the central knob in orthogonal sections).

Although the chemical composition of endothelial caveolae started to yield some insights, the molecular components of transendothelial channels and fenestrae remained elusive. The chemistry of these endothelial microdomains has been investigated with nonspecific “general” probes (charged molecules and lectins alone or in combination with various degrading enzymes), which yielded some information on the surface charge, type of molecules conferring the charge, and type of glycan antennae found on the glycoproteins and glycolipids. No specific component of the fenestral or transendothelial channels diaphragms has been identified so far.

Proteins reported to be contained within caveolae include G protein-coupled receptors (GPCR) (Ostrom R S, et al., J Pharmacol Exp Ther August 2000;294(2):407-12), scavenger receptor class B type I (SR-BI) (Krieger M Annu Rev Biochem 1999;68:523-58), Monocarboxylate transporters (Bonen A, Med Sci Sports Exerc April 2000;32(4):778-89), endothelial NOS (eNOS) (Kone B C Acta Physiol Scand January 2000;168(1):27-31). IP3 receptor-like protein, Ca2+ ATPase, several PKC isoforms. (Isshiki M, et al., Cell Calcium November 1999;26(5):201-8). and GPI-anchored molecules (Martins V R, Braz J Med Biol Res July 1999;32(7):853-9).

The disclosed NOV7 nucleic acid of the invention encoding a PV-1-like protein includes the nucleic acid whose sequence is provided in Table 7A, 7C, or 7E or a fragment thereof. The invention also includes a mutant or variant nucleic acid any of whose bases may be changed from the corresponding base shown in Table 7A, 7C, or 7E while still encoding a protein that maintains its PV-1-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 30% percent of the bases may be so changed.

The disclosed NOV7 protein of the invention includes the PV-1-like protein whose sequence is provided in Table 7B, 7D, or 7F. The invention also includes a mutant or variant protein any of whose residues may be changed from the corresponding residue shown in Table 7B, 7D, or 7F while still encoding a protein that maintains its PV-1-like activities and physiological functions, or a functional fragment thereof. In the mutant or variant protein, up to about 40% percent of the residues may be so changed.

The protein similarity information, expression pattern, and map location for the PV-1-like protein and nucleic acid (NOV7) disclosed herein suggest that NOV7 may have important structural and/or physiological functions characteristic of the PV-1-like family. Therefore, the NOV7 nucleic acids and proteins of the invention are useful in potential diagnostic and therapeutic applications. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo.

The NOV7 nucleic acids and proteins of the invention are useful in potential diagnostic and therapeutic applications implicated in various diseases and disorders described below and/or other pathologies. For example, the compositions of the present invention will have efficacy for treatment of patients suffering from Cerebellar ataxia, pure; Episodic ataxia, type 2; Hemiplegic migraine, familial; Leigh syndrome; Spinocerebellar ataxia-6; Psoriasis, susceptibility to; Autoimmune disease, Asthma, Emphysema, Scleroderma, allergy, ARDS, Von Hippel-Lindau (VHL) syndrome, Alzheimer's disease, Stroke, Tuberous sclerosis, hypercalceimia, Parkinson's disease, Huntington's disease, Cerebral palsy, Epilepsy, Lesch-Nyhan syndrome, Multiple sclerosis, Ataxia-telangiectasia, Leukodystrophies, Behavioral disorders, Addiction, Anxiety, Pain, Neuroprotection, Muscular dystrophy, Myasthenia gravis, Hemophilia, Hypercoagulation, Idiopathic thrombocytopenic purpura, Immunodeficiencies, Graft vesus host, Von Hippel-Lindau (VHL) syndrome, Cirrhosis, Transplantation, Cardiomyopathy, Atherosclerosis, Hypertension, Congenital heart defects, Aortic stenosis, Atrial septal defect (ASD), Atrioventricular (A-V) canal defect, Ductus arteriosus, Pulmonary stenosis, Subaortic stenosis, Ventricular septal defect (VSD), valve diseases, Scieroderma, Obesity, Transplantation; fertility; cancer; Renal artery stenosis, Interstitial nephritis, Glomerulonephritis, Polycystic kidney disease, Systemic lupus erythematosus, Renal tubular acidosis, IgA nephropathy, Hypercalceimia, Lesch-Nyhan syndrome, Adrenoleukodystrophy, Congenital Adrenal Hyperplasia, Xerostomia; tooth decay and other dental problems; Inflammatory bowel disease, Diverticular disease, Pancreatitis, and/or other pathologies/disorders. The NOV7 nucleic acid, or fragments thereof, may further be useful in diagnostic applications, wherein the presence or amount of the nucleic acid or the protein are to be assessed.

NOV7 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV7 protein have multiple hydrophilic regions, each of which can be used as an immunogen. In one embodiment, contemplated NOV7 epitope is from about amino acids 5 to 25. In other embodiments, NOV7 epitope is from about amino acids 50 to 75, from about amino acids 80 to 160, from about amino acids 175 to 275, from about amino acids 280 to 380, or from about amino acids 385 to 430. This novel protein also has value in development of powerful assay system for functional analysis of various human disorders, which will help in understanding of pathology of the disease and development of new drug targets for various disorders.

NOV8

NOV8 includes two novel Papin-like proteins disclosed below. The disclosed proteins have been named NOV8a, and NOV8b.

NOV8a

A disclosed NOV8a nucleic acid of 8640 nucleotides (also referred to as SC134914330_A) encoding a novel papin-like protein is shown in Table 8A. An open reading frame was identified beginning with an ATG initiation codon at nucleotides 101-103 and ending with a TGA codon at nucleotides 8543-8545. A putative untranslated region upstream from the initiation codon and downstream from the termination codon is underlined in Table 8A. The start and stop codons are in bold letters.

TABLE 8A
NOV8a nucleotide sequence (SEQ ID NO: 24).

AGCTGATGATGGCCAGGGACCCCAGGGGACGTGGGGCCCTGTGGGGTCTGGCCCCCAGGAGCAAGACCTCTG
ATGATGCTGGTGTCTGGGAGTGAGCACCATGCCCATCACCCAGGACAATGCCGTGCTGCACCTGCCCCTCCT
CTACCAGTGGCTGCAGAACAGCCTGCAGGAAGGTGGGGATGGGCCGGAGCAGCGGCTCTGCCAGGCGGCCAT
CCAGAAGCTGCAGGAGTACATCCAGCTGAACTTTGCTGTGGATGAGAGTACCGTCCCACCTGATCACAGCCC
CCCCGAAATGGAGATCTGTACTGTGTACCTCACCAAGGAGCTGGGGGACACAGAGACTGTGGGCCTGAGTTT
TGGGAACATCCCTGTTTTCCGGGACTATGGTGAAAAGCGCAGGGGGGGCAAGAAGAGGAAAACCCACCAGGG
TCCTGTGCTGGATGTGGGCTGCATCTGGGTGACAGAGCTGAGGAAGAACAGCCCAGCAGGGAAGAGTGGGAA
GGTCCGACTGCCGGATGAGATCCTCTCACTGAATGGGCAGCTGATGGTTGGAGTTGATGTCAGTGGGGCCAG
TTACCTGGCTGAGCAGTGCTGGAATGGCGGCTTTATCTACCTGATCATGCTGCGTCGCTTTAAGCACAAAGC
CCACTCCACTTATAATGGCAACAGTAGCAACAGCTCTGAACCAGGAGAAACACCTACCTTGGAGCTGGGTGA
CCGAACTGCGAAAAAGGCGAAACGAACCAGAAAGTTTGGGGTCATCTCCAGGCCTCCTGCCAACAAGGCCCC
TGAAGAATCCAAGGGCAGCGCTGGCTGTGAGGTGTCCAGTGACCCCAGCACTGAGCTGGAGAACGGCCTGGA
CCCTGAACTTGGAAACGGCCATGTCTTTCAGCTAGAAAATGGCCCAGATTCTCTCAAGGAGGTGGCTGGACC
CCATCTAGAGAGGTCAGAAGTGGACAGAGGGACAGAGCATAGAATTCCAAAGACAGATGCTCCTCTGACCAC
AAGCAATGACAAACGCCGCTTCTCAAAAGGTGGGAAGACGGACTTCCAATCGAGTGACTGCCTGGCACGGTC
CAAGGAGGAAGTTGGCCGAATATGGAAGATGGAGCTGCTCAAAGAATCGGATGGGCTGGGAATTCAGGTTAG
TGGAGGCCGAGGATCAAAGCGCTCACCTCACGCTATCGTTGTCACTCAAGTGAAGGAAGGAGGTGCCGCTCA
CAGGCTCAGGGATGGCAGGCTGTCCTTAGGAGATGAGCTGCTGGTAATCAATGGTCATTTACTGGTCGGGCT
CTCCCACGAGGAAGCAGTGGCCATTCTTCGCTCCGCCACGGGAATGGTGCAGCTTGTGGTGGCCAGCAAGGT
AGGTGTGCTTTCTGCATTTCAGATGCCTGGGACAGATGAACCCCAAGATGTGTGCGGTGCTGAGGAATCCAA
GGGGAACTTGGAAAGTCCCAAACAGGGCAGCAATAAAATCAAGCTCAAGAGTCGCCTTTCAGGTAGGTGGGG
GCTCTACCTGATGCAGCCTGTCGGGGGTGTACACCGCCTTGAGTCAGTTGAAGAATATAACGAGCTGATGGT
GCGGAATGGGGACCCCCGGATCCGGATGTTGGAGGTCTCCCGAGATGGCCGGAAACACTCCCTCCCGCAGCT
GCTGGACTCTTCCAGTGCCTCACAGGAATACCACATTGTGAAGAAGTCTACCCGCTCCTTAAGCACGACTCA
GGTGGAATCTCCTTGGAGGCTCATTCGGCCATCCGTCATCTCGATCATTGGGTTGTACAAAGAAAAAGGCAA
GGGCCTTGGCTTTAGTATTGCTGGAGGTCGAGACTGCATTCGTGGACAGATGGGGATTTTTGTCAACACCAT
CTTCCCAAATGGATCAGCTGCAGAGGACGGAAGACTTAAAGAAGGTGATGAAATCCTAGATGTAAATGGAAT
ACCAATAAAGGGCTTGACATTTCAAGAAGCCATTCATACCTTTAAGCAAATCCGGAGTGGATTATTTGTTTT
AACGGTACGCACAAAGTTGGTGAGCCCCAGCCTCACACCCTGCTCGACACCCACACACATGAGCAGATCCGC
CTCCCCGAACTTCAATACCAGTGGGGGAGCCTCGGCGGGAGGTTCCGATGAAGGCAGTTCTTCATCCCTGGG
TCGGAAGACCCCTGGGCCCAAGGACAGGATCGTCATGGAAGTAACACTCAACAAAGAGCCAAGAGTTGGATT
AGGCATTGGTGCCTGCTGCTTGGCTCTGGAAAACAGTCCTCCTGGCATCTACATTCACAGCCTTGCTCCAGG
ATCAGTGGCCAAGATGGAGAGCAACCTGTCGCGGGGATCAATCCTGGAAGTGAACTCCGTCAACGTCCGCCA
TGCTGCTTTAAGCAAAGTCCACGCCATCTTGAGTAAATGCCCTCCACGACCCGTTCGCCTTGTCATCGGCCG
GCACCCTAATCCAAAGGTGAATCAGGTTTCCGAGCAGGAAATGGATGAAGTCATAGCACGCAGCACTTATCA
GGAGAGCAAAGAGGCCAATTCCTCTCCTGGCTTAGGTACTGTAATCTCAATCGGATGTTTTCTTCTTCAACA
GGACTCCCTTATTTCTGAATCTGAACTCTCCCAGTACTTTGCCCACGATGTCCCTGGCCCCTTGTCAGACTT
CATGGTGGCCGGTTCTGAGGACGAGGATCACCCGGGAAGTGGCTGCAGCACGTCGGAGGAGGGCAGCCTGCC
TCCCAGCACCTCCACTCACAAGGAGCCTGGAAAACCCAGAGCCAACAGCCTCGTGACTCTTGGGAGCCATCG
GGCTTCTGGGCTCTTCCACAAGCAGGTGACAGTTGCCAGACAAGCCAGTCTCCCCGGAAGCCCACAGGCCCT
CCGAAACCCTCTCCTCCGCCAGAGGAAGGTAGGCTGCTACGATGCCAACGATGCCAGTGATGAGGAAGAGTT
TGACAGAGAAGGGGACTGCATTTCACTCCCAGGGGCCCTCCCGCGTCCCATCAGGCCTCTGTCAGAGGATGA
CCCGAGGCGTGTCTCAATTTCCTCTTCCAAGGGCATGGACGTCCACAACCAAGAGGAACGACCCCGGAAAAC
ACTGGTGAGCAAGGCCATCTCGGCACCTCTTCTTGGTAGCTCAGTGGACTTAGAGGAGAGTATCCCAGAGGG
CATGGTGGATGCTGCGTCCTATGCAGCCAACCTCACGGACTCTGCAGAGGCCCCCAAGGGGAGCCCTGGAAG
CTGGTGGAAGAAGGAACTGTCAGGATCAAGTAGCGCACCCAAATTGGAATACACAGTCCGTACAGACACCCA
GAGTCCGACAAACACTGGGAGCCCCAGTTCCCCCCAGCAAAAAAGTGAAGGCCTGGGCTCCAGGCACAGACC
AGTGGCCAGGGTAAGCCCCCACTGCAAGAGATCCGAGGCTGAGGCCAAGCCCAGTGGCTCACAGACAGTGAA
CCTGACTGGCAGAGCCAATGATCCATGCGATCTGGACTCGAGAGTCCAGGCCACTTCTGTCAAAGTGACTGT
CGCTGGCTTTCAGCCACGTGGAGCTGTGGAGAAGGAATCTCTGGGAAAGCTGACCACTGGAGATGCTTGTGT
CTCTACCAGCTGTGAACTAGCCAGTGCTCTGTCCCATCTGGATGCCAGCCACCTCACAGAGAACCTGCCCAA
AGCTGCATCAGAGCTGGGGCAACAACCCATGACTGAACTGGACAGCTCCTCGGACCTCATCTCTTCCCCAGG
GAAGAAGGGGGCCGCTCATCCTGACCCCAGCAAGACCTCTGTAGACACAGGGAAAGTCAGTCGGCCAGAGAA
TCCCAGCCAGCCTGCATCGCCCACGGTCGCCAAGTGCAAGGCCAGGTCTCCAGTCAGGCTCCCCCATGAGGG
CAGCCCCTCCCCAGGGGAGAAAGCAGCGGCTCCCCCTGACTACAGCAAGACTCGATCAGCATCGGAAACCAG
CACACCCCACAATACCAGGACGGTGGCTGCCCTCAGGGGAGCGGGACCTGGAGCAGAGGGAATGACACCAGC
TGGTGCTGTCCTGCCAGGAGACCCCCTCACATCCCAGGAGCAGAGACAGGGAGCTCCAGGTAACCACAGTAA
GGCTCTGGAAATGACAGGAATCCATGCACCTGAAAGCTCCCAGGAGCCTTCCCTGCTGGAGGGAGCAGATTC
TGTGTCCTCAAGGGCACCGCAGGCCAGCCTCTCCATGCTGCCATCCACTGACAACACCAAAGAAGCATGTGG
CCATGTCTCGGGGCACTGCTGCCCCGGGGGGAGTAGAGAGAGCCCTGTGACGGACATTGACAGCTTCATCAA
GGAGCTGGATGCTTCTGCAGCAAGGTCTCCGTCTTCCCAGACGGGGGACAGTGGCTCTCAGGAGGGCAGTGC
TCAGGGCCACCCACCAGCCGGGGCTGGAGGTGGGAGCTCCTGCCGTGCCGAACCAGTCCCGGGGGGCCAGAC
CTCCTCCCCGAGGAGGGCCTGGGCTGCTGGTGCCCCCGCCTACCCACAATGGGCCTCCCAGCCTTCGGTTTT
AGATTCAATTAATCCCGACAAACATTTTACTGTGAACAAAAACTTTCTGAGCAACTACTCTAGAAATTTTAG
CAGTTTTCATGAAGACAGCACCTCCCTATCAGGCCTGGGTGACAGCACGGAGCCGTCTCTGTCATCCATGTA
TGGCGATGCTGAGGATTCTTCTTCTGACCCTGAGTCACTCACTGAAGCCCCACGAGCTTCTGCCAGGGACGG
CTGGTCCCCTCCTCGTTCCCGTGTGTCTTTGCACAAGGAAGATCCTTCGGAGTCAGAAGAGGAACAGATTGA
GATTTGTTCCACACGTGGCTGCCCCAATCCACCCTCGAGTCCTGCTCATCTTCCCACCCAGGCTGCCATCTG
TCCTGCCTCAGCCAAAGTTCTGTCATTAAAATACAGCACTCCGAGAGAGTCGGTGGCCAGTCCCCGTGAGAA
GGTCGCCTGCTTGCCAGGCTCATACACTTCAGGCCCAGACTCTTCCCAGCCATCATCACTCTTGGAGATGAG
CTCTCAGGAGCATGAAACTCATGCGGACATAAGCACTTCACAGAACCACAGGCCCTCGTGTGCAGAAGAAAC
CACAGAAGTCACCAGCGCTAGCTCAGCCATGGAAAACAGTCCGCTGTCTAAAGTAGCCAGGCATTTTCACAG
TCCGCCCATCATTCTCAGCTCCCCCAACATGGTAAATGGCTTGGAACATGACCTGCTAGATGACGAAACCCT
GAATCAATACGAAACAAGCATTAATGCAGCTGCCAGTCTGTCCTCCTTCAGTGTGGATGTCCCTAAGAATGG
AGAATCTGTTTTGGAAAACCTCCACATCTCTGAAAGTCAAGACCTGGATGACTTGCTACAGAAACCAAAAAT
GATCGCTAGGAGGCCCATCATGGCCTGGTTTAAAGAAATAAATAAACATAACCAAGGCACACATTTGAGGAG
CAAAACCGAGAAGGAACAACCTCTAATGCCTGCCAGAAGTCCCGACTCCAAGATTCAGATGGTGAGTTCAAG
CCAAAAAAAGGGCGTTACTGTGCCTCATAGCCCTCCTCAGCCGAAAACAAACCTGGAAAATAAGGACCTGTC
TAAGAAGAGTCCGGCAGAAATGCTTCTGACTAATGGTCAGAAGGCAAAGTGTGGTCCGAAGCTGAAGACGCT
CAGCCTCAAGGGCAAGGCCAAAGTCAACTCTGAGGCCCCTGCTGCGAATGCTGTGAAGGCTGGGGGGACGGA
CCACAGGAAACCCTTGATCTCACCCCAGACCTCCCACAAAACACTTTCTAAGGCAGTGTCACAGCGGCTCCA
TGTAGCCGACCACGAGGACCCTGACAGAAACACCACAGCTGCCCCCAGGTCCCCCCAGTGTGTGCTGGAAAG
CAAGCCACCTCTTGCCACCTCTGGGCCACTGAAACCCTCAGTGTCTGACACGAGCATCAGGACATTTGTCTC
GCCCCTGACCTCTCCCAAGCCTGTTCCTGAGCAAGGCATGTGGAGCAGGTTCCACATGGCTGTCCTCTCTGA
ACCCGACAGAGGTTGCCCAACCACCCCTAAATCTCCTAAGTGTAGAGCAGAGGGCAGGGCGCCCCGTGCTGA
CTCCGGGCCGGTGAGTCCGGCAGCGTCTAGGAACGGCATGTCCGTCGCAGGGAACAGACAGAGTGAGCCGCG
CCTGGCCAGCCATGTGGCAGCAGACACAGCCCAACCCAGGCCGACTGGCGAAAAAGGAGGCAACATAATGGC
CAGCGATCGCCTCGAAAGAACAAACCAGCTGAAAATCGTGGAGATTTCTGCTGAAGCAGTGTCAGAGACTGT
ATGTGGTAACAAGCCAGCTGAAAGCGACAGACGGGGAGGGTGCTTGGCCCAGGGCAACTGTCAGGAGAAGAG
TGAAATCAGGCTCTATCGCCAGGTCGCAGAATCATCCACAAGTCATCCATCCTCACTCCCATCTCATGCCTC
CCAGGCAGAGCACGAAATGTCACGATCATTCAGCATGGCAAAACTGGCGTCCTCCTCCTCCTCCCTTCAAAC
AGCCATTAGAAAGGCAGAATACTCCCAGGGAAAATCAAGCCTGATGTCAGACTCCCGAGGGGTGCCCAGAAA
CAGCATTCCAGCGGGCCCCTCGGGGGAGGACCATCTCTACTTCACCCCAAGGCCAGCGACCAGGACCTACTC
CATGCCAGCCCAGTTCTCAAGCCATTTTGGACGGGAGGGTCACCCCCCACACAGCCTGGGTCGCTCTCCGGA
CAGCCAGGTCCCTGTGACAAGCAGTGTTGTCCCCGAGGCAAAGGCATCCAGAGGTGGTCTTCCCAGCCTGGC
TAATGGACAGGGCATATATAGTGTAAAGCCGCTGCTGGACACATCGAGGAATCTTCCAGCCACAGATGAAGG
GGATATCATTTCAGTCCAGGAGACGAGCTGCCTAGTCACAGACAAAATCAAAGTCACCAGACGACACTACTG
CTATGAGCAGAACTGGCCCCATGAATCTACCTCATTTTTCTCTGTGAAGCAGCGGATCAAGTCTTTTGAGAA
CCTGGCCAATGCTGACCGGCCTGTAGCCAAGTCCGGGGCTTCCCCATTTTTGTCGGTGAGCTCCAAGCCTCC
CATTGGGAGGCGGTCTTCCGGCAGCATTGTTTCCGGGAGCCTGGGCCACCCAGGTGACGCAGCAGCAAGGTT
GTTGAGACGCAGCTTGAGTTCCTGCAGCGAAAACCAAAGCGAAGCCGGCACCCTCCTGCCCCAGATGGCCAA
GTCTCCCTCAATCATGACACTGACCATCTCTCGGCAGAACCCACCAGAGACCAGTAGCAAGGGCTCTGATTC
GGAACTAAAGAAATCACTTGGTCCTTTGGGAATTCCCACCCCAACGATGACCCTGGCTTCTCCTGTTAAGAG
GAACAAGTCCTCGGTACGCCACACGCAGCCCTCGCCCGTGTCCCGCTCCAAGCTCCACGAGCTGAGAGCCTT
GAGCATGCCTGACCTTGACAAGCTCTGCAGCGAGGATTACTCAGCAGGGCCGAGCGCCGTGCTCTTCAAAAC
TGAGCTGGAGATCACCCCCAGGAGGTCACCTGGCCCTCCTGCTGGAGGCGTTTCGTGTCCCGAGAAGGGCGG
GAACAGGGCCTGTCCAGGAGGAAGTGGCCCTAAAACCAGTGCTGCTGAGACACCCACTTCAGCCAGTGATAC
GGGTGAAGCTGCCCAGGATCTGCCTTTTAGAAGAAGCTGGTCAGTTAATTTGGATCAACTTCTAGTCTCAGC
GGGGGACCAGCAAAGATTACAGTCTGTTTTATCGTCAGTCGGATCGAAATCTACCATCCTAACTCTCATTCA
GGAAGCGAAAGCACAATCAGAGAATGAAGAAGATGTTTGCTTCATAGTCTTGAATAGAAAAGAAGGCTCAGG
TCTGGGATTCAGTCTGGCAGGAGGGACAGATGTCGAGCCAAAATCAATCACGGTCCACAGGGTGTTTTCTCA
GGGGGCGGCTTCTCAGGAAGGGACTATGAACCGAGGGGATTTCCTTCTGTCAGTCAACGGCGCCTCACTGGC
TGGCTTAGCCCACGGGAATGTCCTGAACGTTCTGCACCAGGCACAGCTGCACAAAGATGCCCTCGTGGTCAT
CAAGAAAGGGATGGATCAGCCCAGGCCCTCTGCCCGGCAGGAGCCTCCCACAGCCAATCGGAAGGGTTTGCT
GTCCAGAAAGACCATCCCCCTGGAGCCTGGCATTGGGAGAAGTGTGCCTGTACACGATGCTCTGTGTGTTGA
AGTGCTGAAGACCTCGGCTGGGCTGGGACTGAGTCTGGATGGGGGAAAATCATCGGTGACGGGAGATGGGCC
CTTGGTCATTAAAAGAGTCTACAAAGGTGGTGCGGCTGAACAAGCTGGAATAATAGAAGCTGGAGATGAAAT
TCTTGCTATTAATGGGAAACCTCTGGTTGGGCTCATGCACTTTGATGCCTCGAATATTATGAAGTCTGTCCC
AGAAGGACCTGTGCAGTTATTAATTAGAAAGCATAGGAATTCTTCATGAATTTTAACAAGAATCATTTTCTC
AGTTCTCTTCTTTCTTTAGCAAATCAGAGTGACTTCTTTAAACCACAGGTTGTTGAAATGGCCAACACTGGT

In a search of public sequence databases, the NOV8a nucleic acid sequence, located on chromsome 5 has 997 of 1128 bases (88%) identical to a Papin mRNA from Rattus norvegicus (GENBANK-ID: AF169411). Public nucleotide databases include all GenBank databases and the GeneSeq patent database.

SNP data for NOV1a can be found below in Example 3.

The disclosed NOV8a polypeptide (SEQ ID NO:25) encoded by SEQ ID NO:24 has 2814 amino acid residues and is presented in Table 8B using the one-letter amino acid code. Signal P, Psort and/or Hydropathy results predict that NOV8a has no signal peptide and is likely to be localized in the nucleus with a certainty of 0.7000. In other embodiments, NOV8a may also be localized to the microbody (peroxisome) with a certainty of 0.3000, the mitochondrial matrix space with a certainty of 0.1000, or the lysosome (lumen) with a certainty of 0.1000.

TABLE 8B
Encoded NOV8a protein sequence (SEQ ID NO: 25).

MPITQDNAVLHLPLLYQWLQNSLQEGGDGPEQRLCQAAIQKLQEYIQLNFAVDESTVPPDHSPPEMEICTVY
LTKELGDTETVGLSFGNIPVFGDYCEKRRGGKKRKTHQGPVLDVGCIWVTELRKNSPAGKSGKVRLRDEILS
LNGQLMVGVDVSGASYLAEQCWNCGFIYLIMLRRFKHKAHSTYNGNSSNSSEPGETPTLELGDRTAKKGKRT
RKFGVISRPPANKAPEESKGSAGCEVSSDPSTELEMGLDPELGNGHVFQLENGPDSLKEVAGPHLERSEVDR
GTEHRIPKTDAPLTTSNDKRRFSKGGKTDFQSSDCLARSKEEVGRIWKMELLKESDGLGIQVSGGRGSKRSP
HAIVVTQVKEGGAAHRLRDGRLSLGDELLVINGHLLVGLSHEEAVAILRSATGMVQLVVASKVGVLSAFQMP
GTDEPQDVCGAEESKGNLESPKQGSNKIKLKSRLSGRWGLYLMQPVGGVHRLESVEEYNELMVRNGDPRIRM
LEVSRDGRKHSLPQLLDSSSASQEYHIVKKSTRSLSTTQVESPWRLIRPSVISIIGLYKEKGKGLGFSIAGG
RDCIRGQMGIFVKTIFPNGSAAEDGRLKEGDEILDVNGIPIKGLTFQEAIHTFKQIRSGLFVLTVRTKLVSP
SLTPCSTPTHMSRSASPNFNTSGGASAGGSDEGSSSSLGRKTPGPKDRIVNEVTLNKEPRVGLGIGACCLAL
ENSPPGIYIHSLAPGSVAKMESNLSRGSILEVNSVNVRHAALSKVHAILSKCPPGPVRLVIGRHPNPKVNQV
SEQEMDEVIARSTYQESKEANSSPGLGTVISIGCFLLQQDSLISESELSQYFAHDVPGPLSDFMVAGSEDED
HPGSGCSTSEEGSLPPSTSTHKEPGKPRANSLVTLGSHRASGLFHKQVTVARQASLPGSPQALRNPLLRQRK
VGCYDANDASDEEEFDREGDCISLPGALPGPIRPLSEDDPRRVSISSSKGMDVRNQEERPRKTLVSKAISAP
LLGSSVDLEESIPEGMVDAASYAANLTDSAEAPKGSPGSWWKKELSGSSSAPKLEYTVRTDTQSPTNTGSPS
SPQQKSEGLGSRHRPVARVSPHCIRSEAEAKPSGSQTVNLTGRANDPCDLDSRVQATSVKVTVAGFQPGGAV
EKESLGKLTTGDACVSTSCELASALSMLDASHLTENLPKAASELGQQPMTELDSSSDLISSPGKKGAAHPDP
SKTSVDTGKVSRPENPSQPASPRVAKCKARSPVRLPHEGSPSPGEKAAAPPDYSKTRSASETSTPHNTRRVA
ALRGAGPGAEGMTPAGAVLPGDPLTSQEQRQGAPGNHSKALEMTGIHAPESSQEPSLLEGADSVSSRAPQAS
LSMLPSTDNTKEACGHVSGHCCPGGSRESPVTDIDSFIKELDASAARSPSSQTGDSGSQEGSAQGHPPAGAG
GGSSCRAEPVPGGQTSSPRRAWAAGAPAYPQWASQPSVLDSINPDKHFTVNKNFLSNYSRNFSSFHEDSTSL
SGLGDSTEPSLSSMYGDAEDSSSDPESLTEAPRASARDGWSPPRSRVSLHKEDPSESEEEQIEICSTRGCPN
PPSSPAHLPTQAAICPASAKVLSLKYSTPRESVASPREKVACLPGSYTSGPDSSQPSSLLEMSSQEHETHAD
ISTSQNHRPSCAEETTEVTSASSAMENSPLSKVARHFHSPPIILSSPNMVNGLEHDLLDDETLNQYETSINA
AASLSSFSVDVPKNGESVLENLHISESQDLDDLLQKPKMIARRPIMAWFKEINKHNQGTHLRSKTEKEQPLM
PARSPDSKIQMVSSSQKKGVTVPMSPPQPKTNLENKDLSKKSPAEMLLTNGQKAKCGPKLKRLSLKGKAKVN
SEAPAANAVKAGGTDHRKPLISPQTSHKTLSKAVSQRLHVADHEDPDRNTTAAPRSPQCVLESKPPLATSGP
LKPSVSDTSIRTFVSPLTSPKPVPEQGMWSRFHMAVLSEPDRGCPTTPKSPKCRAEGRAPRADSGPVSPAAS
RNGMSVAGNRQSEPRLASHVAADTAQPRPTGEKGGNIMASDRLERTNQLKIVEISAEAVSETVCGNKPAESD
RRGGCLAQGNCQEKSEIRLYRQVAESSTSHPSSLPSHASQAEQEMSRSFSMAKLASSSSSLQTAIRKAEYSQ
GKSSLMSDSRGVPRNSIPGGPSGEDHLYFTPRPATRTYSMPAQFSSHFGREGHPPHSLGRSRDSQVPVTSSV
VPEAKASRGGLPSLANGQGIYSVKPLLDTSRNLPATDEGDIISVQETSCLVTDKIKVTRRHYCYEQNWPHES
TSFFSVKQRIKSFENLANADRPVAKSGASPFLSVSSKPPIGRRSSGSIVSGSLGHPGDAAARLLRRSLSSCS
ENQSEAGTLLPQMAKSPSIMTLTISRQNPPETSSKGSDSELKKSLGPLGIPTPTMTLASPVKRNKSSVRHTQ
PSPVSRSKLQELRALSMPDLDKLCSEDYSAGPSAVLFKTELEITPRRSPGPPAGGVSCPEKGGNRACPGGSG
PKTSAAETPSSASDTGEAAQDLPFRRSWSVNLDQLLVSAGDQQRLQSVLSSVGSKSTILTLIQEAKAQSENE
EDVCFIVLNRKEGSGLGFSVAGGTDVEPKSITVHRVFSQGAASQEGTMNRGDFLLSVNGASLAGLAHGNVLK
VLHQAQLHKDALVVIKKGMDQPRPSARQEPPTANGKGLLSRKTIPLEPGIGRSVAVHDALCVEVLKTSAGLG
LSLDGGKSSVTGDGPLVIKRVYKGGAAEQAGIIEAGDEILAINGKPLVGLMHFDAWNIMKSVPEGPVQLLIR
KHRNSS

A search of sequence databases reveals that the NOV8a amino acid sequence has 937 of 1741 amino acid residues (53%) identical to, and 1133 of 1741 amino acid residues (65%) similar to, the 2766 amino acid residue Papin protein from Rattus norvegicus (Q9QZR8) (E=0.0), and 122 of 304 amino acid residues (40%) identical to, and 176 of 304 amino acid residues (57%) similar to, the 334 amino acid residue Human interleukin-1 6 monomer (patp:AAW19209 ) (E=1.0e⁻⁴⁶). Amino acid databases include the GenBank databases, SwissProt, PDB, PATP, and PIR. The global sequence homology (as defined by FASTA alignment with the full length sequence of this protein) is 72.943% amino acid homology and 69.689 % amino acid identity. In addition, this protein contains the following protein domains (as defined by Interpro) at the indicated nucleotide positions: PDZ domains (IPR001478) at amino acid positions 336 to 422, 558 to 644, 700 to 784, 2597 to 2681, 2725 to 2810.

NOV8a is expressed in at least the following tissues: Nervous System. Brain. Prosencephalon/Forebrain. Diencephalon. Pituitary Gland; Hematopoietic and Lymphatic System. Hematopoietic Tissues. Lymphoid tissue. Lymph node; Whole Organism. In addition, the sequence is predicted to be expressed in the following tissues because of the expression pattern of (GENBANK-ID: AF169411) a closely related Papin homolog in species Rattus norvegicus: brain. TaqMan data for NOV8 can be found below in Example 2.

NOV8b

A disclosed NOV8b nucleic acid of 8640 nucleotides (also referred to as CG57026-04) encoding a novel papin-like protein is shown in Table 8C. An open reading frame was identified beginning with an ATG initiation codon at nucleotides 101-103 and ending with a TGA codon at nucleotides 8534-8536. A putative untranslated region upstream from the initiation codon and downstream from the termination codon is underlined in Table 8C. The start and stop codons are in bold letters.

TABLE 8C
NOV8b nucleotide sequence (SEQ ID NO: 26).

AGCTGATGATGGCCAGGGACCCCAGGGGACGTGGGGCCCTGTGGGGTCTGGCCCCCAGGAGCAAGACCTCTG
ATGATGCTGGTGTCTGGGAGTGAGCACCATGCCCATCACCCAGGACAATGCCGTGCTGCACCTGCCCCTCCT
CTACCAGTGGCTGCAGAACAGCCTGCAGGAAGGTGGGGATGGGCCGGAGCAGCGGCTCTGCCAGGCGGCCAT
CCAGAAGCTGCAGGAGTACATCCAGCTGAACTTTGCTGTGGATGAGAGTACGGTCCCACCTGATCACAGCCC
CCCCGAAATGGAGATCTGTACTGTGTACCTCACCAACGAGCTGGGGGACACAGAGACTGTGGGCCTGAGTTT
TGGGAACATCCCTGTTTTCGGGGACTATGGTGAAAAGCGCAGGGGGGGCAAGAAGAGGAAAACCCACCAGGG
TCCTGTGCTGGATGTGGGCTGCATCTGGGTGACAGAGCTGAGGAAGAACAGCCCAGCAGGGAAGAGTGGGAA
GGTCCGACTGCGGGATGAGATCCTCTCACTGAATGGGCAGCTGATGGTTGGAGTTGATGTCAGTGGGGCCAG
TTACCTGGCTGAGCAGTGCTGGAATGGCGGCTTTATCTACCTGATCATGCTGCGTCGCTTTAAGCACAAAGC
CCACTCCACTTATAATGGCAACAGTAGCAACAGCTCTGAACCAGGAGAAACACCTACCTTGGAGCTGGGTGA
CCGAACTGCGAAAAAGGGGAAACGAACCAGAAAGTTTGGGGTCATCTCCAGGCCTCCTGCCAACAAGGCCCC
TGAAGAATCCAAGGGCAGCGCTGGCTGTGAGGTGTCCAGTGACCCCAGCACTGAGCTGGAGAACGGCCTGGA
CCCTGAACTTCGAAACGGCCATGTCTTTCAGCTAGAAAATGGCCCAGATTCTCTCAAGGAGGTGGCTGGACC
CCATCTAGAGAGGTCAGAAGTCGACAGAGGGACAGACCATAGAATTCCAAAGACAGATGCTCCTCTGACCAC
AAGCAATGACAAACGCCGCTTCTCAAAAGGTGGGAAGACGGACTTCCAATCGAGTGACTGCCTGGCACGGTC
CAACGAGGAAGTTCGCCGAATATGGAAGATGGAGCTGCTCAAAGAATCGGATGGGCTGGGAATTCAGGTTAG
TGGAGGCCGACGATCAAACCGCTCACCTCACGCTATCGTTGTCACTCAAGTGAAGGAAGGAGGTGCCGCTCA
CAGGCTCAGGGATGGCAGGCTGTCCTTAGGAGATGAGCTGCTGGTAATCAATGGTCATTTACTGGTCGGGCT
CTCCCACGAGGAAGCAGTGGCCATTCTTCGCTCCGCCACGGGAATGGTGCAGCTTGTGGTGGCCAGCAAGGT
AGGTGTGCTTTCTGCATTTCAGATGCCTGGGACAGATGAACCCCAAGATGTGTGCGGTGCTGAGGAATCCAA
GGGGAACTTGGAAAGTCCCAAACAGGGCAGCAATAAAATCAAGCTCAAGAGTCGCCTTTCAGGTAGGTGGGG
GCTCTACCTGATGCAGCCTGTCGGGGGTGTACACCGCCTTGAGTCAGTTGAAGAATATAACGAGCTGATGGT
GCGGAATGGGGACCCCCGGATCCGGATGTTGGAGGTCTCCCGAGATGGCCGGAAACACTCCCTCCCGCAGCT
GCTGGACTCTTCCAGTGCCTCACAGGAATACCACATTGTGAAGAAGTCTACCCGCTCCTTAAGCACGACTCA
GGTGGAATCTCCTCGGAGGCTCATTCGGCCATCCGTCATCTCGATCATTGGGTTGTACAAAGAAAAAGGCAA
GGGCCTTGGCTTTAGTATTGCTGGAGGTCGAGACTGCATTCGTGGACAGATGGGGATTTTTGTCAAGACCAT
CTTCCCAAATGGATCAGCTGCAGAGGACGGAAGACTTAAAGAAGGGGATGAAATCCTAGATGTAAATGGAAT
ACCAATAAAGGGCTTGACATTTCAAGAAGCCATTCATACCTTTAAGCAAATCCGGAGTGGATTATTTGTTTT
AACGGTACGCACAAAGTTGGTGAGCCCCAGCCTCACACCCTGCTCGACACCCACACACATGAGCAGATCCGC
CTCCCCGAACTTCAATACCAGTGGGGGAGCCTCGGCGGGAGGTTCCGATGAAGGCAGTTCTTCATCCCTGGG
TCGGAAGACCCCTGGGCCCAAGGACAGGATCGTCATGGAAGTAACACTCAACAAAGAGCCAAGAGTTGGATT
AGGCATTGGTGCCTGCTGCTTGGCTCTGGAAAACAGTCCTCCTGGCATCTACATTCACAGCCTTGCTCCAGG
ATCAGTGGCCAAGATGGAGAGCAACCTGAGCCGCGGGGATCAAATCCTGGAAGTGAACTCCGTCAACGTCCG
CCATGCTGCTTTAAGCAAAGTCCACGCCATCTTGAGTAAATGCCCTCCAGGACCCGTTCGCCTTGTCATCGG
CCGGCACCCTAATCCAAAGGTTTCCGAGCAGGAAATGGATGAAGTCATAGCACGCAGCACTTATCAGGAGAG
CAAAGAGGCCAATTCCTCTCCTGGCTTACGTACCCCCTTGAAGAGTCCCTCTCTTGCAAAAAAGGACTCCCT
TATTTCTGAATCTGAACTCTCCCAGTACTTTGCCCACGATGTCCCTGGCCCCTTGTCAGACITCATGGTGGT
CGGTTCTGAGGACGAGGATCACCCGGGAAGTGGCTGCAGCACGTCGGAGGAGGGCAGCCTGCCTCCCAGCAC
CTCCACTCACAAGGAGCCTGGAAAACCCAGAGCCAACAGCCTCGTGACTCTTGGGAGCCATCGGGCTTCTGG
GCTCTTCCACAAGCAGGTGACAGTTGCCAGACAAGCCAGTCTCCCCGGAAGCCCACAGGCCCTCCGAAACCC
TCTCCTCCGCCAGAGGAAGGTAGGCTGCTACGATGCCAACGATGCCAGTGATGAGGAAGAGTTTGACAGAGA
AGGGGACTGCATTTCACTCCCAGGGGCCCTCCCGCGTCCCATCAGGCCTCTGTCAGAGGATGACCCGAGGCG
TGTCTCAATTTCCTCTTCCAAGGGCATGGACGTCCACAACCAAGAGGAACGACCCCGGAAAACACTGGTGAG
CAAGGCCATCTCGGCACCTCTTCTTGGTAGCTCAGTGGACTTAGAGGAGAGTATCCCACAGGGCATGGTGGA
TGCTGCGTCCTATGCAGCCAACCTCACGGACTCTGCAGAGGCCCCCAAGGGGAGCCCTGGAAGCTGGTGGAA
GAAGGAACTGTCAGGATCAAGTAGCGCACCCAAATTGGAATACACAGTCCGTACAGACACCCAGAGTCCGAC
AAACACTGGGAGCCCCAGTTCCCCCCAGCAAAAAAGTGAAGGCCTGGGCTCCAGGCACAGACCAGTGGCCAG
GGTAAGCCCCCACTGCAAGAGATCCGAGGCTGAGGCCAAGCCCAGTGGCTCACAGACAGTGAACCTGACTGG
CAGAGCCAATGATCCATGCGATCTGGACTCGAGAGTCCAGGCCACTTCTGTCAAAGTGACTGTCGCTGGCTT
TCAGCCAGGTGGAGCTGTGGAGAAGGAATCTCTGGGAAAGCTGACCACTGGAGATGCTTGTGTCTCTACCAG
CTGTGAACTAGCCAGTGCTCTGTCCCATCTGGATGCCAGCCACCTCACAGAGAACCTGCCCAAAGCTGCATC
AGAGCTGGGGCAACAACCCATGACTGAACTGGACAGCTCCTCGGACCTCATCTCTTCCCCAGGGAAGAAGGG
GGCCGCTCATCCTGACCCCAGCAAGACCTCTGTAGACACAGCGAAAGTCAGTCGGCCAGAGAATCCCAGCCA
GCCTGCATCGCCCAGGGTCGCCAAGTGCAAGGCCAGGTCTCCAGTCAGGCTCCCCCATGAGGGCAGCCCCTC
CCCAGGGGAGAAAGCAGCGGCTCCCCCTGACTACAGCAAGACTCGATCAGCATCGGAAACCAGCACACCCCA
CAATACCAGGAGGGTGGCTGCCCTCAGGGGAGCGGGACCTGGAGCAGAGGGAATGACACCAGCTGGTCCTGT
CCTGCCAGGAGACCCCCTCACATCCCAGGAGCAGAGACAGGGAGCTCCAGGTAACCACAGTAAGGCTCTGGA
AATGACAGGAATCCATGCACCTGAAAGCTCCCAGGAGCCTTCCCTGCTGGAGGGAGCAGATTCTGTGTCCTC
AAGGGCACCGCACGCCAGCCTCTCCATGCTGCCATCCACTGACAACACCAAAGAAGCATGTGGCCATGTCTC
GGGGCACTGCTCCCCGGGGGGGAGTACAGAGAGCCCTGTGACGGACATTGACAGCTTCATCAAGGAGCTGGA
TGCTTCTGCAGCAAGGTCTCCGTCTTCCCAGACGGCGGACAGTGGCTCTCAGGAGGGCAGTGCTCAGGGCCA
CCCACCAGCCGGGGCTGGAGGTGGGAGCTCCTGCCGTGCCGAACCAGTCCCGGGGGGCCAGACCTCCTCCCC
GAGGAGGGCCTGGGCTGCTGGTGCCCCCGCCTACCCACAATGGGCCTCCCAGCCTTCGGTTTTAGATTCAAT
TAATCCCGACAAACATTTTACTGTGAACAAAAACTTTCTGAGCAACTACTCTAGAAATTTTAGCAGTTTTCA
TGAAGACAGCACCTCCCTATCAGGCCTGGGTGACAGCACGGAGCCGTCTCTGTCATCCATGTATGGCGATGC
TGAGGATTCTTCTTCTGACCCTGAGTCACTCACTGAAGCCCCACGAGCTTCTGCCAGGGACGGCTGGTCCCC
TCCTCGTTCCCGTGTGTCTTTGCACAAGGAAGATCCTTCGGAGTCAGAAGAGGAACAGATTGAGATTTGTTC
CACACGTGGCTGCCCCAATCCACCCTCGAGTCCTGCTCATCTTCCCACCCAGGCTGCCATCTGTCCTGCCTC
AGCCAAAGTTCTGTCATTAAAATACAGCACTCCGAGAGAGTCGGTGGCCAGTCCCCGTGAGAAGGTCGCCTG
CTTGCCAGGCTCATACACTTCAGGCCCAGACTCTTCCCACCCATCATCACTCTTCGAGATGAGCTCTCAGGA
GCATGAAACTCATGCCGACATAAGCACTTCACAGAACCACAGGCCCTCGTGTGCAGAAGAAACCACAGAAGT
CACCAGCGCTAGCTCAGCCATGGAAAACAGTCCGCTGTCTAAAGTAGCCAGGCATTTTCACAGTCCGCCCAT
CATTCTCAGCTCCCCCAACATGCTAAATGGCTTGGAACATGACCTGCTAGATGACGAAACCCTGAATCAATA
CGAAACAAGCATTAATGCAGCTGCCAGTCTGTCCTCCTTCAGTGTGGATGTCCCTAAGAATGGAGAATCTGT
TTTGGAAAACCTCCACATCTCTGAAAGTCAAGACCTGGATGACTTGCTACAGAAACCAAAAATGATCGCTAG
GAGGCCCATCATGGCCTGGTTTAAAGAAATAAATAAACATAACCAAGGCACACATTTGAGGAGCAAAACCGA
GAAGGAACAACCTCTAATGCCTGCCAGAAGTCCCGACTCCAAGATTCAGATGGTGAGTTCAAGCCAAAAAAA
GGGCGTTACTGTGCCTCATAGCCCTCCTCAGCCGAAAACAAACCTGGAAAATAAGGACCTGTCTAAGAAGAG
TCCGGCAGAAATGCTTCTGACTAATGGTCAGAAGGCAAAGTGTGGTCCGAAGCTGAAGAGGCTCAGCCTCAA
GGGCAAGGCCAAAGTCAACTCTGAGGCCCCTGCTCCGAATGCTGTGAAGGCTGGCGGGACGGACCACAGGAA
ACCCTTGATCTCACCCCAGACCTCCCACAAAACACTTTCTAAGGCAGTGTCACAGCGGCTCCATGTAGCCGA
CCACGAGGACCCTGACAGAAACACCACAGCTGCCCCCAGGTCCCCCCAGTGTGTGCTGGAAAGCAAGCCACC
TCTTGCCACCTCTGGGCCACTGAAACCCTCAGTGTCTGACACGAGCATCAGGACATTTGTCTCGCCCCTGAC
CTCTCCCAAGCCTGTTCCTGAGCAAGGCATGTGGAGCAGGTTCCACATGGCTGTCCTCTCTGAACCCGACAG
AGGTTGCCCAACCACCCCTAAATCTCCTAAGTGTAGAGCAGAGCGCAGGGCGCCCCGTCCTGACTCCGGGCC
GGTGAGTCCGGCAGCGTCTAGGAACGGCATGTCCGTGGCAGGGAACAGACAGAGTGAGCCGCGCCTGGCCAG
CCATGTGGCAGCAGACACAGCCCAACCCAGGCCGACTGGCGAAAAAGGAGGCAACATAATGGCCAGCGATCG
CCTCGAAAGAACAAACCAGCTGAAAATCGTGGAGATTTCTGCTGAAGCAGTGTCAGAGACTGTATGTGGTAA
CAACCCAGCTGAAAGCGACAGACGGGGAGGGTGCTTGGCCCAGCGCAACTGTCAGGAGAAGAGTGAAATCAG
GCTCTATCGCCAGGTCGCAGAATCATCCACAAGTCATCCATCCTCACTCCCATCTCATGCCTCCCAGGCAGA
GCAGGAAATGTCACGATCATTCAGCATGGCAAAACTGGCGTCCTCCTCCTCCTCCCTTCAAACAGCCATTAC
AAAGGCAGAATACTCCCAGGGAAAATCAAGCCTGATGTCAGACTCCCGAGGGGTGCCCAGAAACAGCATTCC
AGGGGGCCCCTCGGGGGAGGACCATCTCTACTTCACCCCAAGGCCAGCGACCAGGACCTACTCCATGCCAGC
CCAGTTCTCAAGCCATTTTGGACGGGAGGGTCACCCCCCACACAGCCTGGGTCGCTCTCGGGACAGCCAGGT
CCCTGTGACAAGCAGTGTTGTCCCCGAGGCAAAGGCATCCAGAGGTGGTCTTCCCAGCCTGGCTAATGGACA
GGGCATATATAGTGTAAAGCCGCTGCTGGACACATCGAGGAATCTTCCAGCCACAGATCAAGGGGATATCAT
TTCAGTCCAGGAGACGAGCTGCCTAGTCACAGACAAAATCAAAGTCACCAGACGACACTACTGCTATGAGCA
GAACTGGCCCCATGAATCTACCTCATTTTTCTCTGTGAAGCAGCGGATCAAGTCTTTTGAGAACCTGGCCAA
TGCTGACCGGCCTGTAGCCAAGTCCGGGGCTTCCCCATTTTTGTCGGTGAGCTCCAAGCCTCCCATTGGGAG
GCGGTCTTCCGGCAGCATTGTTTCCGGGAGCCTGGGCCACCCAGGTGACGCAGCAGCAAGGTTGTTGAGACG
CAGCTTGAGTTCCTGCAGCGAAAACCAAAGCGAAGCCGGCACCCTCCTGCCCCAGATGGCCAAGTCTCCCTC
AATCATGACACTGACCATCTCTCGGCAGAACCCACCAGAGACCAGTAGCAAGGGCTCTGATTCGGAACTAAA
GAAATCACTTGGTCCTTTGGGAATTCCCACCCCAACGATGACCCTGGCTTCTCCTGTTAAGAGGAACAAGTC
CTCGGTACGCCACACGCAGCCCTCGCCCGTGTCCCGCTCCAAGCTCCAGGAGCTGAGAGCCTTGAGCATGCC
TGACCTTGACAAGCTCTGCAGCGAGGATTACTCAGCAGGGCCGAGCGCCGTGCTCTTCAAAACTGAGCTGGA
GATCACCCCCAGGAGGTCACCTGGCCCTCCTGCTGGAGGCGTTTCGTGTCCCGAGAAGGGCGGGAACAGGGC
CTGTCCAGGAGGAAGTGGCCCTAAAACCAGTGCTGCTGAGACACCCAGTTCAGCCAGTGATACGGGTGAAGC
TGCCCAGGATCTGCCTTTTAGAAGAAGCTGGTCAGTTAATTTGGATCAACTTCTAGTCTCAGCGGGGGACCA
GCAAAGATTACAGTCTGTTTTATCGTCAGTGGGATCGAAATCTACCATCCTAACTCTCATTCAGGAAGCGAA
AGCACAATCAGAGAATGAAGAAGATGTTTGCTTCATAGTCTTGAATAGAAAAGAAGGCTCAGGTCTGGGATT
CAGTGTGGCAGGAGGGACAGATGTGGAGCCAAAATCAATCACGGTCCACAGGGTGTTTTCTCAGGGGGCGGC
TTCTCAGGAAGGGACTATGAACCGAGGGGATTTCCTTCTGTCAGTCAACGGCGCCTCACTGGCTGGCTTAGC
CCACGGGAATGTCCTGAAGGTTCTGCACCAGGCACAGCTGCACAAAGATGCCCTCGTGGTCATCAAGAAAGG
GATGGATCAGCCCAGGCCCTCTGCCCGGCAGGAGCCTCCCACAGCCAATGGGAAGGGTTTGCTGTCCAGAAA
GACCATCCCCCTGGAGCCTGGCATTGGGAGAAGTGTGGCTGTACACGATGCTCTGTGTGTTGAAGTGCTGAA
GACCTCGGCTGGGCTGGGACTGAGTCTGGATGGGGGAAAATCATCGGTGACGGGAGATGGGCCCTTGGTCAT
TAAAAGAGTGTACAAAGGTGGTGCGGCTGAACAAGCTGGAATAATAGAAGCTGGAGATGAAATTCTTGCTAT
TAATGGGAAACCTCTGGTTGGGCTCATGCACTTTGATGCCTGGAATATTATGAAGTCTGTCCCAGAAGGACC
TGTGCAGTTATTAATTAGAAAGCATAGGAATTCTTCATGAATTTTAACAAGAATCATTTTCTCAGTTCTCTT
CTTTCTTTAGCAAATCAGAGTGACTTCTTTAAACCACAGGTTGTTGAAATGGCCAACACTGGTACAGACACG

In a search of public sequence databases, the NOV8a nucleic acid sequence, located on chromsome 5 has 5828 of 5941 bases (98%) identical to a gb:GENBANK-ID:AF338650|acc:AF338650.1 mRNA from Homo sapiens (Homo sapiens PDZ domain-containing protein AIPC (AIPC) mRNA, complete cds) (E=0.0). Public nucleotide databases include all GenBank databases and the GeneSeq patent database.

The disclosed NOV8b polypeptide (SEQ ID NO:27) encoded by SEQ ID NO:26 has 2811 amino acid residues and is presented in Table 8D using the one-letter amino acid code. Signal P, Psort and/or Hydropathy results predict that NOV8b has no signal peptide and is likely to be localized in the nucleus with a certainty of 0.7000. In other embodiments, NOV8b is also likely to be localized to the microbody (peroxisome) with a certainty of 0.3000, the mitochondrial matrix space with a certainty of 0.1000, and to the lysosome (lumen) with a certainty of 0.1000.

TABLE 8D
Encoded NOV8b protein sequence (SEQ ID NO: 27).

MPITQDNAVLHLPLLYQWLQNSLQEGGDGPEQRLCQAAIQKLQEYIQLNFAVDESTVPPDHSPPEMEICTVY
LTKELGDTETVGLSFGNIPVFGDYGEKRRGGKKRKTHQGPVLDVGCIWVTELRKNSPAGKSGKVRLRDEILS
LNGQLMVGVDVSGASYLAEQCWNGGPIYLIMLRRFKHKAHSTYNGNSSNSSEPGETPTLELGDRTAKKGKRT
RKFGVISRPPAFKAPEESKGSAGCEVSSDPSTELENGLDPELGNGHVFQLENGPDSLKEVAGPHLERSEVDR
GTEHRIPKTDAPLTTSNDKRRFSKGGKTDFQSSDCLARSKEEVGRIWKMELLKESDGLGIQVSGGRGSKRSP
HAIVVTQVKEGGAAMRLRDGRISLGDELLVINGHLLVGLSHEEAVAILRSATGMVQLVVASKVGVLSAFQMP
GTDEPQDVCGAEESKGNLESPKQGSNKIKLKSRLSGRWGLYLMQPVGGVHRLESVEEYNELMVRNGDPRIRM
LEVSRDGRKHSLPQLLDSSSASQEYHIVKKSTRSLSTTQVESPRRLIRPSVISIIGLYKEKGKGLGFSIAGG
RDCIRGQMGIFVKTIFPNGSAAEDGRLKEGDEILDVNGIPIKGLTFQEAIHTFKQIRSGLFVLTVRTKLVSP
SLTPCSTPTHMSRSASPNFNTSGGASAGGSDEGSSSSLGRKTPGPKDRIVMEVTLNKEPRVGLGIGACCLAL
ENSPPGIYIHSLAPGSVAKMESNLSRGDQILEVNSVNVRHAALSKVHAILSKCPPGPVRLVIGRHPNPKVSE
QEMDEVIARSTYQESKEANSSPGLGTPLKSPSLAKKDSLISESELSQYFAHDVPGPLSDFMVVGSEDEDHPG
SGCSTSEEGSLPPSTSThKEPCKPRANSLVTLGSHRASGLFHKQVTVARQASLPGSPQALRNPLLRQRKVGC
YDANDASDEEEFDREGDCISLPGALPGPIRPLSEDDPRRVSISSSKGMDVHNQEERPRKTLVSKAISAPLLG
SSVDLEESIPEGMVDAASYAANLTDSAEAPKGSPGSWWKKELSGSSSAPKLEYTVRTDTQSPTNTGSPSSPQ
QKSEGLGSRHRPVARVSPHCKRSEAEAKPSGSQTVNLTGRANDPCDLDSRVQATSVKVTVAGFQPGGAVEKE
SLGKLTTGDACVSTSCELASALSHLDASHLTENLPKAASELGQQPMTELDSSSDLISSPGKKGAAMPDPSKT
SVDTGKVSRPENPSQPASPRVAKCKARSPVRLPHEGSPSPGEKAAAPPDYSKTRSASETSTPHNTRRVAALR
GAGPGAEGMTPAGAVLPGDPLTSQEQRQGAPGNHSKALEMTGIHAPESSQEPSLLEGADSVSSRAPQASLSM
LPSTDNTKEACGHVSGHCCPGGSRESPVTDIDSFIKELDASAARSPSSQTGDSGSQEGSAQGHPPAGAGGGS
SCRAEPVPGGQTSSPRRAWAAGAPAYPQWASQPSVLDSINPDKHFTVNKNFLSNYSRNFSSFMEDSTSLSGL
GDSTEPSLSSMYGDAEDSSSDPESLTEAPRASARDGWSPPRSRVSLHKEDPSESEEEQIEICSTRGCPNPPS
SPAHLPTQAAICPASAKVLSLKYSTPRESVASPREKVACLPGSYTSGPDSSQPSSLLENSSQEHETHADIST
SQNHRPSCAEETTEVTSASSAMENSPLSKVARHFHSPPIILSSPNNVNGLEHDLLDDETLNQYETSINAAAS
LSSFSVDVPKNGESVLENLHISESQDLDDLLQKPKMIARRPIMAWFKEINKHNQGThLESKTEKEQPLMPAR
SPDSKIQMVSSSQKKGVFVPHSPPQPKTNLENKDLSKKSPAEMLLTNGQKAKCGPKLKRLSLKGKAKVNSEA
PAANAVKAGGTDHRKPLISPQTSHKTLSKAVSQRLHVADHEDPDRNTTAAPRSPQCVLESKPPLATSGPLKP
SVSDTSIRTFVSPLTSPKPVPEQGMWSRFHMAVLSEPDRGCPTTPKSPKCRAEGRAPRADSGPVSPAASRNG
MSVAGNRQSEPRLASHVAADTAQPRPTGEKGGNIMASDRLERTNQLKIVEISAEAVSETVCGNKPAESDRRG
GCLAQGNCQEKSEIRLYRQVAESSTSHPSSLPSHASQAEQEMSRSFSMAKLASSSSSLQTAIRKAEYSQGKS
SLMSDSRGVPRNSIPGGPSGEDHLYFTPRPATRTYSMPAQFSSHFGREGHPPHSLGRSRDSQVPVTSSVVPE
AKASRGGLPSLANGOGIYSVKPLLDTSRNLPATDEGDIISVQETSCLVTDKIKVTRRHYCYEQNWPHESTSF
FSVKQRIKSFENLANADRPVAKSGASPFLSVSSKPPIGRRSSGSIVSGSLGHPGDAAAPLLRRSLSSCSENQ
SEAGTLLPQMAXSPSIMTLTISRQNPPETSSKGSDSELKKSLGPLGIPTPTMTLASPVKRNKSSVRHTQPSP
VSRSKLQELRALSMPDLDKLCSEDYSAGPSAVLPKTELEITPRRSPGPPAGGVSCPEKGGNRACPGGSGPKT
SAAETPSSASDTGEAAQDLPFRRSWSVNLDQLLVSAGDQQRLQSVLSSVGSKSTILTLIQEAKAQSENEEDV
CFIVLNRKEGSGLGFSVAGGTDVEPKSITVHRVFSQGAASQEGTMNRGDFLLSVNGASLAGLAHGNVLKVLH
QAQLHKDALVVIKKGMDQPRPSAROEPPTANGKGLLSRKTIPLEPGIGRSVAVHDALCVEVLKTSAGLGLSL
DGGKSSVTGDGPLVIKRVYKGGAAEQAGIIEAGDEILAINGKPLVGLMHFDAWNIMKSVPEGPVQLLIRKHR
NSS

A search of sequence databases reveals that the NOV8a amino acid sequence has 2017 of 2045 amino acid residues (98%) identical to, and 2022 of 2045 amino acid residues (98%) similar to, the 2641 amino acid residue ptnr:TREMBLNEW-ACC:AAK07661 protein from Homo sapiens (Human) (PDZ DOMAIN-CONTAINING PROTEIN AIPC) (E=0.0). Public amino acid databases include the GenBank databases, SwissProt, PDB and PIR.

NOV8b is expressed in at least the following tissues: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea and uterus. Expression information was derived from the tissue sources of the sequences that were included in the derivation of the sequence of CuraGen Acc. No. CG57026-04.The sequence is predicted to be expressed in the following tissues because of the expression pattern of (GENBANK-ID: gb:GENBANK-ID:AF338650|acc:AF338650.1) a closely related Homo sapiens PDZ domain-containing protein AIPC (AIPC) mRNA, complete cds homolog in species Homo sapiens: prostate. TaqMan data for NOV8b can be found below in Example 2.

The NOV8a, and 8b proteins are very closely homologous as as shown in the alignment in Table 8E.

[image]

[image]

[image]

[image]

Homologies to either of the above NOV8 proteins will be shared by the other NOV8 protein insofar as they are homologous to each other as shown above. Any reference to NOV8 is assumed to refer to both of the NOV8 proteins in general, unless otherwise noted.

The disclosed NOV8 polypeptide has homology to the amino acid sequences shown in the BLASTP data listed in Table 8F.

TABLE 8F
BLAST results for NOV8

| Gene Index/ | | Length | Identity | Positives | |
|---|---|---|---|---|---|
| Identifier | Protein/Organism | (aa) | (%) | (%) | Expect |
| gi\|15295903\|ref\|XP_{—} | similar to | 1788 | 1712/1741 | 1716/1741 | 0.0 |
| 043060.2\| | NONE_RETURNED | | (98%) | (98%) | |
| | (R. norvegicus) | | | | |
| | [Homo | | | | |
| | sapiens] | | | | |
| gi\|2224541\|dbj\|BAA20760.1\|P | KIAA0300 [Homo | 1608 | 1608/1608 | 1608/1608 | 0.0 |
| | sapiens] | | (100%) | (100%) | |
| gi\|12621106\|ref\|NP_{—} | PAPIN [Rattus | 2766 | 1906/2843 | 2147/2843 | 0.0 |
| 075229.1\| | norvegicus] | | (67%) | (75%) | |
| gi\|12751452\|gb\|AAK07661.1\| | PDZ domain- | 2641 | 2256/2343 | 2275/2343 | 0.0 |
| AF338650_1 | containing | | (96%) | (96%) | |
| | protein AIPC | | | | |
| | [Homo sapiens] | | | | |
| gi\|12861607\|dbj\|BAB32241.1\| | putative [Mus | 364 | 314/387 | 337/387 | e−157 |
| | musculus] | | (81%) | (86%) | |

The homology between these and other sequences is shown graphically in the ClustalW analysis shown in Table 8G. In the ClustalW alignment of the NOV8 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

[image]

[image]

[image]

[image]

[image]

[image]

[image]

[image]

[image]

Table 8H-J lists the domain description from DOMAIN analysis results against NOV8. This indicates that the NOV8 sequence has properties similar to those of other proteins known to contain this domain.

TABLE 8H

Domain Analysis of NOV8
gnl|Smart|smart00228, PDZ, Domain present in PSD-95, Dlg, and ZO-1/2.;
Also called DHR (Dlg homologous region) or GLGF (relatively well
conserved tetrapeptide in these domains). Some PDZs have been shown to
bind C-terminal polypeptides; others appear to bind internal (non-C-
terminal) polypeptides. Different PDZs possess different binding
specificities.
(SEQ ID NO: 86)
CD-Length = 86 residues, 95.3% aligned
Score = 69.3 bits (168), Expect = 3e−12

| | | | |
|---|---|---|---|
| Query: | 333 | RIWKMELLKESDGLGIQVSGGRGSKRSPHAIVVTQVKEGGAAHRLRDGRLSLGDELLVIN | 392 |
| | | +\|\| \| \|\|\| + \|\|+ \| +\|\|+ \| \| \| + \| \| \|\| +\| +\| | |
| Sbjct: | 1 | EPRLVELEKGGGGLGFSLVGGKDSGDGG--VVVSSVVPGSPAAK--AG-LKPGDVILEVN | 55 |
| Query: | 393 | GHLLVGLSHEEAVAILRSATGMVQLVV | 419 |
| | | \| + \|\|+\| \|\|\| +\|+ \| \| \| \| \| | |
| Sbjct: | 56 | GTSVEGLTHLEAVDLLKEAGGKVTLTV | 82 |

TABLE 8I

Domain Analysis of NOV8
gnl|Smart|smart00228, PDZ, Domain present in PSD-95, Dlg, and ZO-1/2.;
Also called DHR (Dlg homologous region) or GLGF (relatively well
conserved tetrapeptide in these domains). Some PDZs have been shown to
bind C-terminal polypeptides; others appear to bind internal (non-C-
terminal) polypeptides. Different PDZs possess different binding
specificities.
(SEQ ID NO: 86)
CD-Length = 86 residues, 96.5% aligned
Score = 66.2 bits (160), Expect = 2e−11

| | | | |
|---|---|---|---|
| Query: | 556 | ISIIGLYKEKGKGLGFSIAGGRDCIRGQMGIFVKTIFPNGSAAEDGRLKEGDEILDVNGI | 615 |
| | | ++ \| \| \| \|\|\|\|\|+ \|\|+\| \| \|+ \| ++ \| \|\|+ \| \|\| \|\| \|\|+\|\|\| | |
| Sbjct: | 2 | PRLVELEKGGG-GLGFSLVGGKD--SGDGGVVVSSVVPGSPAAKAG-LKPGDVILEVNGT | 57 |
| Query: | 616 | PIKGLTFQEAIHTFKQIRSGLFVLTVR | 642 |
| | | ++\|\|\| \|\|+ \|+ + + +\| | |
| Sbjct: | 58 | SVEGLTMLEAVDLLKEAGGKVTLTVLR | 84 |

TABLE 8J

Domain Analysis of NOV8
gnl|Smart|smart00228, PDZ, Domain present in PSD-95, Dlg, and ZO-1/2.;
Also called DHR (Dlg homologous region) or GLGF (relatively well
conserved tetrapeptide in these domains) . Some PDZs have been shown to
bind C-terminal polypeptides; others appear to bind internal (non-C-
terminal) polypeptides. Different PDZs possess different binding
specificities.
(SEQ ID NO: 86)
CD-Length = 86 residues, 97.7% aligned
Score = 60.1 bits (144), Expect = 2e−09

| | | | |
|---|---|---|---|
| Query: | 2597 | FIVLNRKEGSGLGFSVAGGTDVEPKSITVHRVFSQGAASQEGTMNRGDFLLSVNGASLAG | 2656 |
| | | +\| \| \| \|\|\|\|\|+ \|\| \| + \| \| \|++ \| + \|\| +\| \|\|\| \|+ \| | |
| Sbjct: | 3 | RLVELEKGGGCLGFSLVGGKDSGDGGVVVSSVVPGSPAAKAG-LKPGDVILEVNGTSVEG | 61 |
| Query: | 2657 | LAHGNVLKVLHQAQLHKDALVVIKKG | 2682 |
| | | \| \| + +\| +\| \| \| \|++ \| | |
| Sbjct: | 62 | LTHLEAVDLLKEAG-GKVTLTVLRGG | 86 |

Proteins belonging to the IGFBP-ALS family of proteins play an important role in regulating the levels of circulating hormones. The acid labile subunit of the complex plays an important role in regulating the stability of the complex and ensuring high levels of circulating hormones that are regulated by the IGFBP family of proteins. This protein also has a leucine rich repeat that is a common domain in many proteins that are important for the developing embryo. As a result this protein may play an important role in development and disease.

Insulin-like growth factors (IGFs) I and II are important regulators of cell proliferation and differentiation (Ueki I et al., Proc Natl Acad Sci USA 2000 Jun. 6;97(12):6868-73). After birth, plasma IGFs, representing mostlyliver-derived IGFs, circulate in ternary complexes of 150 kDa consisting of one molecule each of IGF, IGF-binding protein (IGFBP) 3, and an acid labile subunit (ALS). Onset of ALS synthesis after birth is the primary factor driving the formation of ternary complexes. Capture of IGFs by ALS is thought to allow the development of a plasma reservoir without negative effects such as hypoglycemia and cell proliferation. To evaluate the importance of ALS and ternary complexes, mice have been created in which the ALS gene has been inactivated. The mutation was inherited in a Mendelian manner, without any effects on survival rates and birth weights. A growth deficit was observed in null mice after 3 weeks of life and reached 13% by 10 weeks. This modest phenotype was observed despite reductions of 62 and 88% in the concentrations of plasma IGF-I and IGFBP-3, respectively. Increased turnover accounted for these reductions because indices of synthesis in liver and kidney were not decreased. Surprisingly, absence of ALS did not affect glucose and insulin homeostasis. Therefore, ALS is required for postnatal accumulation of IGF-I and IGFBP-3 but, consistent with findings supporting a predominant role for locally produced IGF-I, is not critical for growth. This model should be useful to determine whether presence of ALS is needed for other actions of liver-derived IGF-I and for maintenance of homeostasis in presence of high circulating levels of IGF-II.

In the circulation, insulin-like growth factor-I (IGF-I) is bound in a trimeric complex of 150 kDa with IGF binding protein-3 (IGFBP-3) and the acid-labile subunit (ALS). (Moller S et al., J Hepatol March 2000;32(3):441-6). Whereas circulating IGF-I and IGFBP-3 are reported to be low in patients with chronic liver failure, the level of ALS has not been described in relation to hepatic dysfunction. The aim of the present study was therefore to measure circulating and hepatic venous concentrations of ALS in relation to hepatic function and the IGF axis.

The insulin-like growth factor (IGF) binding proteins (IGFBPs) were initially identified as carrier proteins for IGF-I and IGF-II in a variety of biologic fluids (Rosenfeld R G et al., Pediatrics October 1999;104(4 Pt 2):1018-21). Their presumed function was to protect IGF peptides from degradation and clearance, increase the half-life of the IGFs, and deliver them to appropriate tissue receptors. The concept of IGFBPs as simple carrier proteins has been complicated, however, by a number of observations: 1) the six IGFBPs vary in their tissue expression and their regulation by other hormones and growth factors; 2) the IGFBPs are subjected to proteolytic degradation, thereby altering their affinities for the IGFs; 3) IGFBP-3 and IGFBP-5, in addition to binding IGFs, also can associate with an acid-labile subunit, thereby increasing further the half-life of the IGFs; 4) in addition to modifying the access of IGF peptides to IGF and insulin receptors, several of the IGFBPs may be capable of increasing IGF action; 5) some of the IGFBPs may be capable of IGF-independent regulation of cell growth; 6) some of the IGFBPs are associated with cell membranes or possibly with membrane receptors; and 7) some of the IGFBPs have nuclear recognition sites and may be found within the nucleus. Additionally, a number of cDNAs identified recently have been found to encode proteins that bind IGFs, but with substantially lower affinities than is the case with IGFBPs. The N-terminal regions of the predicted proteins are structurally homologous to the classic IGFBPs, with conservation of the cysteine-rich region. These observations suggest that these low-affinity binders are members of an IGFBP superfamily, capable of regulating cell growth by both IGF-dependent and IGF-independent mechanisms insulin-like growth factor, insulin-like growth factor binding proteins.

Total IGF-I level in serum is a sensitive index during growth hormone (GH) replacement therapy of adults, since GH stimulates the hepatic expressions of both insulin-like growth factor (IGF-I) and acid-labile subunit (ALS) and the major part of IGF-I in the circulation is found in a ternary complex together with ALS and IGFBP-3 (Hall K et al., J Endocrinol Invest 1999;22(5 Suppl):48-57). However, other regulators of the proteins constituting the ternary complex may influence IGF-I levels. In healthy subjects the serum IGF-I levels are low at birth, rise during childhood, with peak levels during puberty, and decline with increasing age. This pattern has been attributed to the age-dependent GH production, but it is unknown whether the wide range of IGF-I levels within each age interval is due to GH production or GH sensitivity. In elderly twins approximately 60% of IGF-I levels are genetically determined. The remaining environmental dependency of IGF-I is partly due to nutrition. Both caloric and protein content of the diet is of importance. Thus, low IGF-I levels are found in GH deficient patients as well as in patients with GH resistance due to malnutrition or GH receptor defects. It is essential that IGF-I determination is performed by assays in which IGFBPs do not interfere, and that IGF-I concentration is evaluated in relation to age, i.e. expressed in SD score, and the number of individuals constituting the reference intervals improves the sensitivity and specificity. Although determination of IGF-I is recommended in assessing GH deficiency in children, its diagnostic value in patients with adult onset of GH deficiency is not agreed upon. In the age group above 40-80 years many patients with pituitary/hypothalamic disorders and GH peaks below 3 microg/l during provocation tests have normal IGF-I levels. It is not clarified, whether the IGF-I levels within normal range for age is due to endogenous basal GH production being sufficient or other factors stimulating IGF-I, IGFBP-3 or ALS expressions.

Circulating insulin-like growth factors (IGFs) represent an important pool of potential hypoglycemic activity, which is largely inhibited by their sequestration in a heterotrimeric complex comprising growth factor, IGF-binding protein-3 (IGFBP-3), and acid-labile subunit (ALS) (Baxter R C Metabolism October 1995;44(10 Suppl 4):12-7). Less than 1% of total IGFs circulate in the free form, yet even this amount might contribute significantly to circulating insulin-like activity. The ternary binding protein complex appears to inhibit insulin-like activity of bound IGFs by preventing their egress from the circulation. Although the integrity of this complex might be affected by limited proteolysis of IGFBP-3 in pregnancy and catabolic conditions, the evidence that this increases IGF bioavailability, and thus hypoglycemic potential, is as yet unclear. However, in patients with IGF-II-secreting tumors, hypoglycemia may result from a failure of the ternary complex to adequately sequester the IGFs. Improvement in complex formation, by treatment with corticosteroids or growth hormone, alleviates the hypoglycemia, even if (as seen with growth hormone treatment) IGF-II hypersecretion persists. In these patients, blood glucose levels are inversely correlated with IGFBP-2 levels, suggesting that this protein might play a part in transporting IGFs to their target tissues. Conversely, ALS levels correlate positively with blood glucose, emphasizing the importance of the ternary complex in preventing hypoglycemia. Unlike the other IGF-binding proteins, IGFBP-1 is acutely regulated in the circulation, in a manner consistent with its acting as a glucose counterregulator. It might act in this way by inhibiting the activity of free IGFs in the circulation.

Leucine-rich repeats (LRRs) are relatively short motifs (22-28 residues in length) found in a variety of cytoplasmic, membrane and extracellular proteins (InterPro). Although these proteins are associated with widely different functions, a common property involves protein-protein interaction. Little is known about the 3D structure of LRRs, although it is believed that they can form amphipathic structures with hydrophobic surfaces capable of interacting with membranes. In vitro studies of a synthetic LRR from Drosophila Toll protein have indicated that the peptides form gels by adopting beta-sheet structures that form extended filaments. These results are consistent with the idea that LRRs mediate protein-protein interactions and cellular adhesion. Other functions of LRR-containing proteins include, for example, binding to enzymes and vascular repair. The 3-D structure of ribonuclease inhibitor, a protein containing 15 LRRs, has been determined, revealing LRRs to be a new class of alpha/beta fold. LRRs form elongated non-globular structures and are often flanked by cysteine rich domains.

The disclosed NOV8 nucleic acid of the invention encoding a papin-like protein includes the nucleic acid whose sequence is provided in Table 8A and C, or a fragment thereof. The invention also includes a mutant or variant nucleic acid any of whose bases may be changed from the corresponding base shown in Table 8A and C while still encoding a protein that maintains its papin-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 12% percent of the bases may be so changed.

The disclosed NOV8 protein of the invention includes the papin-like protein whose sequence is provided in Table 8B and D. The invention also includes a mutant or variant protein any of whose residues may be changed from the corresponding residue shown in Table 2 while still encoding a protein that maintains its papin-like activities and physiological functions, or a functional fragment thereof. In the mutant or variant protein, up to about 43% percent of the residues may be so changed.

The invention further encompasses antibodies and antibody fragments, such as F_{ab} or (F_{ab})_{2,} that bind immunospecifically to any of the proteins of the invention.

The above defined information for this invention suggests that this papin-like protein (NOV8) may function as a member of a “papin family”. Therefore, the NOV8 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV8 nucleic acids and proteins of the invention are useful in potential therapeutic applications implicated in cancer including but not limited to Inflamation, Autoimmune disorders, Aging and Cancer. For example, a cDNA encoding the papin-like protein (NOV8) may be useful in gene therapy, and the papin-like protein (NOV8) may be useful when administered to a subject in need thereof. By way of nonlimiting example, the compositions of the present invention will have efficacy for treatment of patients suffering from cancer, cystitis, incontinence, fertility, cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation recovery. The NOV8 nucleic acid encoding papin-like protein, and the papin-like protein of the invention, or fragments thereof, may further be useful in diagnostic applications, wherein the presence or amount of the nucleic acid or the protein are to be assessed.

NOV8 nucleic acids and polypeptides are further useful in the generation of antibodies that bind immuno-specifically to the novel NOV8 substances for use in therapeutic or diagnostic methods. These antibodies may be generated according to methods known in the art, using prediction from hydrophobicity charts, as described in the “Anti-NOVX Antibodies” section below. The disclosed NOV8 protein has multiple hydrophilic regions, each of which can be used as an immunogen. In one embodiment, a contemplated NOV8 epitope is from about amino acids 10 to 50. In another embodiment, a NOV8 epitope is from about amino acids 80 to 120. In additional embodiments, NOV8 epitopes are from about amino acids 180 to 220, from about amino acids 230 to 300, from about amino acid 330 to 350, from about amino acid 370 to 400, from about amino acid 480 to 540, from about amino acid 550 to 560, and from about amino acids 620 to 840. These novel proteins can be used in assay systems for functional analysis of various human disorders, which will help in understanding of pathology of the disease and development of new drug targets for various disorders.

NOVX Nucleic Acids and Polypeptides

One aspect of the invention pertains to isolated nucleic acid molecules that encode NOVX polypeptides or biologically active portions thereof. Also included in the invention are nucleic acid fragments sufficient for use as hybridization probes to identify NOVX-encoding nucleic acids (e.g., NOVX mRNAs) and fragments for use as PCR primers for the amplification and/or mutation of NOVX nucleic acid molecules. As used herein, the term “nucleic acid molecule” is intended to include DNA molecules (e.g. cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

An NOVX nucleic acid can encode a mature NOVX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full-length gene product, encoded by the corresponding gene. Alternatively, it may be defined as the polypeptide, precursor or proprotein encoded by an ORF described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+l to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

The term “probes”, as utilized herein, refers to nucleic acid sequences of variable length, preferably between at least about 10 nucleotides (nt), 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single- or double-stranded and designed to have specificity in PCR, membrane-based hybridization technologies, or ELISA-like technologies.

The term “isolated” nucleic acid molecule, as utilized herein, is one, which is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated NOVX nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the invention, e.g., a nucleic acid molecule having the nucleotide sequence SEQ ID NOS:1, 3, 4, 6, 8, 10, 12, 14, 16, 18, 20, 22, 24, and 26, or a complement of this aforementioned nucleotide sequence, can be isolated using standard molecular biology techniques and the sequence information provided herein. Using all or a portion of the nucleic acid sequence of SEQ ID NOS:1, 3, 4, 6, 8, 10, 12, 14, 16, 18, 20, 22, 24, and 26 as a hybridization probe, NOVX molecules can be isolated using standard hybridization and cloning techniques (e.g., as described in Sambrook, et al., (eds.), MOLECULAR CLONING: A LABORATORY MANUAL 2^{nd} Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to NOVX nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides SEQ ID NOS:1, 3, 4, 6, 8, 10, 12, 14, 16, 18, 20, 22, 24, and 26, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NOS:1, 3,4, 6, 8, 10, 12, 14, 16, 18, 20, 22, 24, and 26, or a portion of this nucleotide sequence (e.g., a fragment that can be used as a probe or primer or a fragment encoding a biologically-active portion of an NOVX polypeptide). A nucleic acid molecule that is complementary to the nucleotide sequence shown SEQ ID NOS:1, 3, 4, 6, 8, 10, 12, 14, 16, 18, 20, 22, 24, or 26 is one that is sufficiently complementary to the nucleotide sequence shown SEQ ID NOS:1, 3, 4, 6, 8, 10, 12, 14, 16, 18, 20, 22, 24, or 26 that it can hydrogen bond with little or no mismatches to the nucleotide sequence shown SEQ ID NOS:1, 3, 4, 6, 8, 10, 12, 14, 16, 18, 20, 22, 24, and 26, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of NOVX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for an NOVX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human NOVX protein. Homologous nucleic acid sequences include those nucleic acid sequences that encode conservative amino acid substitutions (see below) in SEQ ID NOS:1, 3, 4, 6, 8, 10, 12, 14, 16, 18, 20, 22, 24, and 26, as well as a polypeptide possessing NOVX biological activity. Various biological activities of the NOVX proteins are described below.

An NOVX polypeptide is encoded by the open reading frame (“ORF”) of an NOVX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bona fide cellular protein, a minimum size requirement is often set, e.g., a stretch of DNA that would encode a protein of 50 amino acids or more.

The nucleotide sequences determined from the cloning of the human NOVX genes allows for the generation of probes and primers designed for use in identifying and/or cloning NOVX homologues in other cell types, e.g. from other tissues, as well as NOVX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence SEQ ID NOS:1, 3, 4, 6, 8, 10, 12, 14, 16, 18, 20, 22, 24, or 26; or an anti-sense strand nucleotide sequence of SEQ ID NOS:1, 3, 4, 6, 8, 10, 12, 14, 16, 18, 20, 22, 24, or 26; or of a naturally occurring mutant of SEQ ID NOS:1, 3, 4, 6, 8, 10, 12, 14, 16, 18, 20, 22, 24, and 26.

Probes based on the human NOVX nucleotide sequences can be used to detect transcripts or genomic sequences encoding the same or homologous proteins. In various embodiments, the probe further comprises a label group attached thereto, e.g. the label group can be a radioisotope, a fluorescent compound, an enzyme, or an enzyme co-factor. Such probes can be used as a part of a diagnostic test kit for identifying cells or tissues which mis-express an NOVX protein, such as by measuring a level of an NOVX-encoding nucleic acid in a sample of cells from a subject e.g., detecting NOVX mRNA levels or determining whether a genomic NOVX gene has been mutated or deleted.

“A polypeptide having a biologically-active portion of an NOVX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of NOVX” can be prepared by isolating a portion SEQ ID NOS:1, 3, 4, 6, 8, 10, 12, 14, 16, 18, 20, 22, 24, or 26, that encodes a polypeptide having an NOVX biological activity (the biological activities of the NOVX proteins are described below), expressing the encoded portion of NOVX protein (e.g., by recombinant expression in vitro) and assessing the activity of the encoded portion of NOVX.

NOVX Nucleic Acid and Polypeptide Variants

The invention further encompasses nucleic acid molecules that differ from the nucleotide sequences shown in SEQ ID NOS:1, 3, 4, 6, 8, 10, 12, 14, 16, 18, 20, 22, 24, and 26 due to degeneracy of the genetic code and thus encode the same NOVX proteins as that encoded by the nucleotide sequences shown in SEQ ID NOS:1, 3, 4, 6, 8, 10, 12, 14, 16, 18, 20, 22, 24, and 26. In another embodiment, an isolated nucleic acid molecule of the invention has a nucleotide sequence encoding a protein having an amino acid sequence shown in SEQ ID NOS:2, 5, 7, 9, 11, 13, 15, 17, 19, 21, 23, 25, or 27.

In addition to the human NOVX nucleotide sequences shown in SEQ ID NOS:1, 3, 4, 6, 8, 10, 12, 14, 16, 18, 20, 22, 24, and 26, it will be appreciated by those skilled in the art that DNA sequence polymorphisms that lead to changes in the amino acid sequences of the NOVX polypeptides may exist within a population (e.g., the human population). Such genetic polymorphism in the NOVX genes may exist among individuals within a population due to natural allelic variation. As used herein, the terms “gene” and “recombinant gene” refer to nucleic acid molecules comprising an open reading frame (ORF) encoding an NOVX protein, preferably a vertebrate NOVX protein. Such natural allelic variations can typically result in 1-5% variance in the nucleotide sequence of the NOVX genes. Any and all such nucleotide variations and resulting amino acid polymorphisms in the NOVX polypeptides, which are the result of natural allelic variation and that do not alter the functional activity of the NOVX polypeptides, are intended to be within the scope of the invention.

Moreover, nucleic acid molecules encoding NOVX proteins from other species, and thus that have a nucleotide sequence that differs from the human SEQ ID NOS:1, 3, 4, 6, 8, 10, 12, 14, 16, 18, 20, 22, 24, and 26 are intended to be within the scope of the invention. Nucleic acid molecules corresponding to natural allelic variants and homologues of the NOVX cDNAs of the invention can be isolated based on their homology to the human NOVX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NOS:1, 3, 4, 6, 8, 10, 12, 14, 16, 18, 20, 22, 24, and 26. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding NOVX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. An isolated nucleic acid molecule of the invention that hybridizes under stringent conditions to the sequences SEQ ID NOS:1, 3, 4, 6, 8, 10, 12, 14, 16, 18, 20, 22, 24, and 26, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NOS:1, 3, 4, 6, 8, 10, 12, 14, 16, 18, 20, 22, 24, and 26, or fragments, analogs or derivatives thereof, under conditions of moderate stringency is provided. A non-limiting example of moderate stringency hybridization conditions are hybridization in 6×SSC, 5× Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known within the art. See, e.g., Ausubel, et al. (eds.), 1993, CURRENT PROTOCOLS IN MOLECULAR BIOLOGY, John Wiley & Sons, NY, and Kriegler, 1990; GENE TRANSFER AND EXPRESSION, A LABORATORY MANUAL, Stockton Press, NY.

In a third embodiment, a nucleic acid that is hybridizable to the nucleic acid molecule comprising the nucleotide sequences SEQ ID NOS:1, 3,4, 6, 8, 10, 12, 14, 16, 18, 20, 22, 24, and 26, or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981. Proc Natl Acad Sci USA 78: 6789-6792.

Conservative Mutations

In addition to naturally-occurring allelic variants of NOVX sequences that may exist in the population, the skilled artisan will further appreciate that changes can be introduced by mutation into the nucleotide sequences SEQ ID NOS:1, 3, 4, 6, 8, 10, 12, 14, 16, 18, 20, 22, 24, and 26, thereby leading to changes in the amino acid sequences of the encoded NOVX proteins, without altering the functional ability of said NOVX proteins. For example, nucleotide substitutions leading to amino acid substitutions at “non-essential” amino acid residues can be made in the sequence SEQ ID NOS:2, 5, 7, 9, 11, 13, 15, 17, 19, 21, 23, 25, or 27. A “non-essential” amino acid residue is a residue that can be altered from the wild-type sequences of the NOVX proteins without altering their biological activity, whereas an “essential” amino acid residue is required for such biological activity. For example, amino acid residues that are conserved among the NOVX proteins of the invention are predicted to be particularly non-amenable to alteration. Amino acids for which conservative substitutions can be made are well-known within the art.

Another aspect of the invention pertains to nucleic acid molecules encoding NOVX proteins that contain changes in amino acid residues that are not essential for activity. Such NOVX proteins differ in amino acid sequence from SEQ ID NOS:1, 3,4, 6, 8, 10, 12, 14, 16, 18, 20, 22, 24, and 26 yet retain biological activity. In one embodiment, the isolated nucleic acid molecule comprises a nucleotide sequence encoding a protein, wherein the protein comprises an amino acid sequence at least about 45% homologous to the amino acid sequences SEQ ID NOS:2, 5, 7, 9, 11, 13, 15, 17, 19, 21, 23, 25, and 27. Preferably, the protein encoded by the nucleic acid molecule is at least about 60% homologous to SEQ ID NOS:2, 5, 7, 9, 11, 13, 15, 17, 19, 21, 23, 25, and 27; more preferably at least about 70% homologous SEQ ID NOS:2, 5, 7, 9, 11, 13, 15, 17, 19, 21, 23, 25, or 27; still more preferably at least about 80% homologous to SEQ ID NOS:2, 5, 7, 9, 11, 13, 15, 17, 19, 21, 23, 25, or 27; even more preferably at least about 90% homologous to SEQ ID NOS:2, 5, 7, 9, 11, 13, 15, 17, 19, 21, 23, 25, or 27; and most preferably at least about 95% homologous to SEQ ID NOS:2, 5, 7, 9, 11, 13, 15, 17, 19, 21, 23, 25, or 27.

An isolated nucleic acid molecule encoding an NOVX protein homologous to the protein of SEQ ID NOS:2, 5, 7, 9, 11, 13, 15, 17, 19, 21, 23, 25, or 27 can be created by introducing one or more nucleotide substitutions, additions or deletions into the nucleotide sequence of SEQ ID NOS:1, 3, 4, 6, 8, 10, 12, 14, 16, 18, 20, 22, 24, and 26, such that one or more amino acid substitutions, additions or deletions are introduced into the encoded protein.

Mutations can be introduced into SEQ ID NOS:1, 3, 4, 6, 8, 10, 12, 14, 16, 18, 20, 22, 24, and 26 by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the NOVX protein is replaced with another amino acid residue from the same side chain family. Alternatively, in another embodiment, mutations can be introduced randomly along all or part of an NOVX coding sequence, such as by saturation mutagenesis, and the resultant mutants can be screened for NOVX biological activity to identify mutants that retain activity. Following mutagenesis SEQ ID NOS:1, 3, 4, 6, 8, 10, 12, 14, 16, 18, 20, 22, 24, and 26, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, VLIM, HFY, wherein the letters within each group represent the single letter amino acid code.

In one embodiment, a mutant NOVX protein can be assayed for (i) the ability to form protein:protein interactions with other NOVX proteins, other cell-surface proteins, or biologically-active portions thereof, (ii) complex formation between a mutant NOVX protein and an NOVX ligand; or (iii) the ability of a mutant NOVX protein to bind to an intracellular target protein or biologically-active portion thereof; (e.g. avidin proteins).

In yet another embodiment, a mutant NOVX protein can be assayed for the ability to regulate a specific biological function (e.g., regulation of insulin release).

Antisense Nucleic Acids

Another aspect of the invention pertains to isolated antisense nucleic acid molecules that are hybridizable to or complementary to the nucleic acid molecule comprising the nucleotide sequence of SEQ ID NOS:1, 3, 4, 6, 8, 10, 12, 14, 16, 18, 20, 22, 24, and 26, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire NOVX coding strand, or to only a portion thereof. Nucleic acid molecules encoding fragments, homologs, derivatives and analogs of an NOVX protein of SEQ ID NOS:2, 5, 7, 9, 11, 13, 15, 17, 19, 21, 23, 25, or 27, or antisense nucleic acids complementary to an NOVX nucleic acid sequence of SEQ ID NOS:1, 3, 4, 6, 8, 10, 12, 14, 16, 18, 20, 22, 24, and 26, are additionally provided.

In one embodiment, an antisense nucleic acid molecule is antisense to a “coding region” of the coding strand of a nucleotide sequence encoding an NOVX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the NOVX protein. The term “noncoding region” refers to 5′ and 3′ sequences which flank the coding region that are not translated into amino acids (i.e., also referred to as 5′ and 3′ untranslated regions).

Given the coding strand sequences encoding the NOVX protein disclosed herein, antisense nucleic acids of the invention can be designed according to the rules of Watson and Crick or Hoogsteen base pairing. The antisense nucleic acid molecule can be complementary to the entire coding region of NOVX mRNA, but more preferably is an oligonucleotide that is antisense to only a portion of the coding or noncoding region of NOVX mRNA. For example, the antisense oligonucleotide can be complementary to the region surrounding the translation start site of NOVX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an NOVX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An a-anomeric nucleic acid molecule forms specific double-stranded hybrids with complementary RNA in which, contrary to the usual β-units, the strands run parallel to each other. See, e.g., Gaultier, et al., 1987. Nucl. Acids Res. 15: 6625-6641. The antisense nucleic acid molecule can also comprise a 2′-o-methylribonucleotide (See, e.g., Inoue, et al. 1987. Nucl. Acids Res. 15: 6131-6148) or a chimeric RNA-DNA analogue (See, e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave NOVX mRNA transcripts to thereby inhibit translation of NOVX mRNA. A ribozyme having specificity for an NOVX-encoding nucleic acid can be designed based upon the nucleotide sequence of an NOVX cDNA disclosed herein (i.e., SEQ ID NOS:1, 3, 4, 6, 8, 10, 12, 14, 16, 18, 20, 22, 24, and 26). For example, a derivative of a Tetrahymena L-19 IVS RNA can be constructed in which the nucleotide sequence of the active site is complementary to the nucleotide sequence to be cleaved in an NOVX-encoding mRNA. See, e.g., U.S. Pat. No. 4,987,071 to Cech, et al. and U.S. Pat. No. 5,116,742 to Cech, et al. NOVX mRNA can also be used to select a catalytic RNA having a specific ribonuclease activity from a pool of RNA molecules. See, e.g., Bartel et al., (1993) Science 261:1411-1418.

Alternatively, NOVX gene expression can be inhibited by targeting nucleotide sequences complementary to the regulatory region of the NOVX nucleic acid (e.g., the NOVX promoter and/or enhancers) to form triple helical structures that prevent transcription of the NOVX gene in target cells. See, e.g., Helene, 1991. Anticancer Drug Des. 6: 569-84; Helene, et al. 1992. Ann. N. Y Acad. Sci. 660: 27-36; Maher, 1992. Bioassays 14: 807-15.

In various embodiments, the NOVX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, et al, 1996. supra; Perry-O'Keefe, et al., 1996. Proc. Natl. Acad. Sci. USA 93: 14670-14675.

PNAs of NOVX can be used in therapeutic and diagnostic applications. For example, PNAs can be used as antisense or antigene agents for sequence-specific modulation of gene expression by, e.g., inducing transcription or translation arrest or inhibiting replication. PNAs of NOVX can also be used, for example, in the analysis of single base pair mutations in a gene (e.g., PNA directed PCR clamping; as artificial restriction enzymes when used in combination with other enzymes, e.g., S₁ nucleases (See, Hyrup, et al., 1996.supra); or as probes or primers for DNA sequence and hybridization (See, Hyrup, et al., 1996, supra; Perry-O'Keefe, et al., 1996. supra).

In another embodiment, PNAs of NOVX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of NOVX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleobases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

NOVX Polypeptides

A polypeptide according to the invention includes a polypeptide including the amino acid sequence of NOVX polypeptides whose sequences are provided in SEQ ID NOS:2, 5, 7, 9, 11, 13, 15, 17, 19, 21, 23, 25, or 27. The invention also includes a mutant or variant protein any of whose residues may be changed from the corresponding residues shown in SEQ ID NOS:2, 5, 7, 9, 11, 13, 15, 17, 19, 21, 23, 25, or 27 while still encoding a protein that maintains its NOVX activities and physiological functions, or a functional fragment thereof.

In general, an NOVX variant that preserves NOVX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated NOVX proteins, and biologically-active portions thereof, or derivatives, fragments, analogs or homologs thereof. Also provided are polypeptide fragments suitable for use as immunogens to raise anti-NOVX antibodies. In one embodiment, native NOVX proteins can be isolated from cells or tissue sources by an appropriate purification scheme using standard protein purification techniques. In another embodiment, NOVX proteins are produced by recombinant DNA techniques. Alternative to recombinant expression, an NOVX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the NOVX protein is derived, or substantially free from chemical precursors or other chemicals when chemically synthesized. The language “substantially free of cellular material” includes preparations of NOVX proteins in which the protein is separated from cellular components of the cells from which it is isolated or recombinantly-produced. In one embodiment, the language “substantially free of cellular material” includes preparations of NOVX proteins having less than about 30% (by dry weight) of non-NOVX proteins (also referred to herein as a “contaminating protein”), more preferably less than about 20% of non-NOVX proteins, still more preferably less than about 10% of non-NOVX proteins, and most preferably less than about 5% of non-NOVX proteins. When the NOVX protein or biologically-active portion thereof is recombinantly-produced, it is also preferably substantially free of culture medium, i.e., culture medium represents less than about 20%, more preferably less than about 10%, and most preferably less than about 5% of the volume of the NOVX protein preparation.

The language “substantially free of chemical precursors or other chemicals” includes preparations of NOVX proteins in which the protein is separated from chemical precursors or other chemicals that are involved in the synthesis of the protein. In one embodiment, the language “substantially free of chemical precursors or other chemicals” includes preparations of NOVX proteins having less than about 30% (by dry weight) of chemical precursors or non-NOVX chemicals, more preferably less than about 20% chemical precursors or non-NOVX chemicals, still more preferably less than about 10% chemical precursors or non-NOVX chemicals, and most preferably less than about 5% chemical precursors or non-NOVX chemicals.

Biologically-active portions of NOVX proteins include peptides comprising amino acid sequences sufficiently homologous to or derived from the amino acid sequences of the NOVX proteins (e.g., the amino acid sequence shown in SEQ ID NOS:2, 5, 7, 9, 11, 13, 15, 17, 19, 21, 23, 25, or 27) that include fewer amino acids than the full-length NOVX proteins, and exhibit at least one activity of an NOVX protein. Typically, biologically-active portions comprise a domain or motif with at least one activity of the NOVX protein. A biologically-active portion of an NOVX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native NOVX protein.

In an embodiment, the NOVX protein has an amino acid sequence shown SEQ ID NOS:2, 5, 7, 9, 11, 13, 15, 17, 19, 21, 23, 25, or 27. In other embodiments, the NOVX protein is substantially homologous to SEQ ID NOS:2, 5, 7, 9, 11, 13, 15, 17, 19, 21, 23, 25, or 27, and retains the functional activity of the protein of SEQ ID NOS:2, 5, 7, 9, 11, 13, 15, 17, 19, 21, 23, 25, or 27, yet differs in amino acid sequence due to natural allelic variation or mutagenesis, as described in detail, below. Accordingly, in another embodiment, the NOVX protein is a protein that comprises an amino acid sequence at least about 45% homologous to the amino acid sequence SEQ ID NOS:2, 5, 7, 9, 11, 13, 15, 17, 19, 21, 23, 25, or 27, and retains the functional activity of the NOVX proteins of SEQ ID NOS:2, 5, 7, 9, 11, 13, 15, 17, 19, 21, 23, 25, or 27.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NOS:1, 3, 4, 6, 8, 10, 12, 14, 16, 18, 20, 22, 24, and 26.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides NOVX chimeric or fusion proteins. As used herein, an NOVX “chimeric protein” or “fusion protein” comprises an NOVX polypeptide operatively-linked to a non-NOVX polypeptide. An “NOVX polypeptide” refers to a polypeptide having an amino acid sequence corresponding to an NOVX protein SEQ ID NOS:2, 5, 7, 9, 11, 13, 15, 17, 19, 21, 23, 25, or 27, whereas a “non-NOVX polypeptide” refers to a polypeptide having an amino acid sequence corresponding to a protein that is not substantially homologous to the NOVX protein, e.g., a protein that is different from the NOVX protein and that is derived from the same or a different organism. Within an NOVX fusion protein the NOVX polypeptide can correspond to all or a portion of an NOVX protein. In one embodiment, an NOVX fusion protein comprises at least one biologically-active portion of an NOVX protein. In another embodiment, an NOVX fusion protein comprises at least two biologically-active portions of an NOVX protein. In yet another embodiment, an NOVX fusion protein comprises at least three biologically-active portions of an NOVX protein. Within the fusion protein, the term “operatively-linked” is intended to indicate that the NOVX polypeptide and the non-NOVX polypeptide are fused in-frame with one another. The non-NOVX polypeptide can be fused to the N-terminus or C-terminus of the NOVX polypeptide.

In one embodiment, the fusion protein is a GST-NOVX fusion protein in which the NOVX sequences are fused to the C-terminus of the GST (glutathione S-transferase) sequences. Such fusion proteins can facilitate the purification of recombinant NOVX polypeptides.

In another embodiment, the fusion protein is an NOVX protein containing a heterologous signal sequence at its N-terminus. In certain host cells (e.g., mammalian host cells), expression and/or secretion of NOVX can be increased through use of a heterologous signal sequence.

In yet another embodiment, the fusion protein is an NOVX-immunoglobulin fusion protein in which the NOVX sequences are fused to sequences derived from a member of the immunoglobulin protein family. The NOVX-immunoglobulin fusion proteins of the invention can be incorporated into pharmaceutical compositions and administered to a subject to inhibit an interaction between an NOVX ligand and an NOVX protein on the surface of a cell, to thereby suppress NOVX-mediated signal transduction in vivo. The NOVX-immunoglobulin fusion proteins can be used to affect the bioavailability of an NOVX cognate ligand. Inhibition of the NOVX ligand/NOVX interaction may be useful therapeutically for both the treatment of proliferative and differentiative disorders, as well as modulating (e.g. promoting or inhibiting) cell survival. Moreover, the NOVX-immunoglobulin fusion proteins of the invention can be used as immunogens to produce anti-NOVX antibodies in a subject, to purify NOVX ligands, and in screening assays to identify molecules that inhibit the interaction of NOVX with an NOVX ligand.

An NOVX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). An NOVX-encoding nucleic acid can be cloned into such an expression vector such that the fusion moiety is linked in-frame to the NOVX protein.

NOVX Agonists and Antagonists

The invention also pertains to variants of the NOVX proteins that function as either NOVX agonists (i.e., mimetics) or as NOVX antagonists. Variants of the NOVX protein can be generated by mutagenesis (e.g., discrete point mutation or truncation of the NOVX protein). An agonist of the NOVX protein can retain substantially the same, or a subset of, the biological activities of the naturally occurring form of the NOVX protein. An antagonist of the NOVX protein can inhibit one or more of the activities of the naturally occurring form of the NOVX protein by, for example, competitively binding to a downstream or upstream member of a cellular signaling cascade which includes the NOVX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the NOVX proteins.

Variants of the NOVX proteins that function as either NOVX agonists (i.e., mimetics) or as NOVX antagonists can be identified by screening combinatorial libraries of mutants (e.g., truncation mutants) of the NOVX proteins for NOVX protein agonist or antagonist activity. In one embodiment, a variegated library of NOVX variants is generated by combinatorial mutagenesis at the nucleic acid level and is encoded by a variegated gene library. A variegated library of NOVX variants can be produced by, for example, enzymatically ligating a mixture of synthetic oligonucleotides into gene sequences such that a degenerate set of potential NOVX sequences is expressible as individual polypeptides, or alternatively, as a set of larger fusion proteins (e.g., for phage display) containing the set of NOVX sequences therein. There are a variety of methods which can be used to produce libraries of potential NOVX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NOVX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

Polypeptide Libraries

In addition, libraries of fragments of the NOVX protein coding sequences can be used to generate a variegated population of NOVX fragments for screening and subsequent selection of variants of an NOVX protein. In one embodiment, a library of coding sequence fragments can be generated by treating a double stranded PCR fragment of an NOVX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with S₁ nuclease, and ligating the resulting fragment library into an expression vector. By this method, expression libraries can be derived which encodes N-terminal and internal fragments of various sizes of the NOVX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NOVX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify NOVX variants. See, e.g., Arkin and Yourvan, 1992. Proc. Natl. Acad Sci. USA 89: 7811-7815; Delgrave, et al., 1993. Protein Engineering 6:327-331.

Anti-NOVX Antibodies

Also included in the invention are antibodies to NOVX proteins, or fragments of NOVX proteins. The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab′} and F_{(ab′)2} fragments, and an F_{ab} expression library. In general, an antibody molecule obtained from humans relates to any of the classes IgG, IgM, IgA, IgE and Ig, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated NOVX-related protein of the invention may be intended to serve as an antigen, or a portion or fragment thereof, and additionally can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of NOVX-related protein that is located on the surface of the protein, e.g., a hydrophilic region. A hydrophobicity analysis of the human NOVX-related protein sequence will indicate which regions of a NOVX-related protein are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each of which is incorporated herein by reference in its entirety. Antibodies that are specific for one or more domains within an antigenic protein, or derivatives, fragments, analogs or homologs thereof, are also provided herein.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow and Lane, 1988, Cold Spring Harbor Laboratory Press, Cold Spring Harbor, N.Y., incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, MONOCLONAL ANTIBODIES: PRINCIPLES AND PRACTICE, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., MONOCLONAL ANTIBODY PRODUCTION TECHNIQUES AND APPLICATIONS, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Preferably, antibodies having a high degree of specificity and a high binding affinity for the target antigen are isolated.

After the desired hybridoma cells are identified, the clones can be subcloned by limiting dilution procedures and grown by standard methods. Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Human Antibodies

Fully human antibodies relate to antibody molecules in which essentially the entire sequences of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al, (Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker, and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., 1991 EMBO J., 10:3655-3659.

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)2 molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5): 1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fc regions and can thereby have enhanced complement lysis and ADCC capabilities. See Stevenson et al., Anti-Cancer Drug Design, 3: 219-230 (1989).

Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol)propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl)hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

In one embodiment, methods for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of an NOVX protein is facilitated by generation of hybridomas that bind to the fragment of an NOVX protein possessing such a domain. Thus, antibodies that are specific for a desired domain within an NOVX protein, or derivatives, fragments, analogs or homologs thereof, are also provided herein.

Anti-NOVX antibodies may be used in methods known within the art relating to the localization and/or quantitation of an NOVX protein (e.g., for use in measuring levels of the NOVX protein within appropriate physiological samples, for use in diagnostic methods, for use in imaging the protein, and the like). In a given embodiment, antibodies for NOVX proteins, or derivatives, fragments, analogs or homologs thereof, that contain the antibody derived binding domain, are utilized as pharmacologically-active compounds (hereinafter “Therapeutics”).

An anti-NOVX antibody (e.g., monoclonal antibody) can be used to isolate an NOVX polypeptide by standard techniques, such as affinity chromatography or immunoprecipitation. An anti-NOVX antibody can facilitate the purification of natural NOVX polypeptide from cells and of recombinantly-produced NOVX polypeptide expressed in host cells. Moreover, an anti-NOVX antibody can be used to detect NOVX protein (e.g., in a cellular lysate or cell supernatant) in order to evaluate the abundance and pattern of expression of the NOVX protein. Anti-NOVX antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

NOVX Recombinant Expression Vectors and Host Cells

Another aspect of the invention pertains to vectors, preferably expression vectors, containing a nucleic acid encoding an NOVX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., NOVX proteins, mutant forms of NOVX proteins, fusion proteins, etc.).

The recombinant expression vectors of the invention can be designed for expression of NOVX proteins in prokaryotic or eukaryotic cells. For example, NOVX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 3140), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the NOVX expression vector is a yeast expression vector. Examples of vectors for expression in yeast Saccharomyces cerivisae include pYepSec 1 (Baldari, et al., 1987. EMBO J. 6: 229-234), pMFa (Kurian and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, NOVX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banedi, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to NOVX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, NOVX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding NOVX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) NOVX protein. Accordingly, the invention further provides methods for producing NOVX protein using the host cells of the invention. In one embodiment, the method comprises culturing the host cell of invention (into which a recombinant expression vector encoding NOVX protein has been introduced) in a suitable medium such that NOVX protein is produced. In another embodiment, the method further comprises isolating NOVX protein from the medium or the host cell.

Transgenic NOVX Animals

The host cells of the invention can also be used to produce non-human transgenic animals. For example, in one embodiment, a host cell of the invention is a fertilized oocyte or an embryonic stem cell into which NOVX protein-coding sequences have been introduced. Such host cells can then be used to create non-human transgenic animals in which exogenous NOVX sequences have been introduced into their genome or homologous recombinant animals in which endogenous NOVX sequences have been altered. Such animals are useful for studying the function and/or activity of NOVX protein and for identifying and/or evaluating modulators of NOVX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous NOVX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing NOVX-encoding nucleic acid into the male pronuclei of a fertilized oocyte (e.g., by microinjection, retroviral infection) and allowing the oocyte to develop in a pseudopregnant female foster animal. The human NOVX cDNA sequences SEQ ID NOS:1, 3, 4, 6, 8, 10, 12, 14, 16, 18, 20, 22, 24, and 26 can be introduced as a transgene into the genome of a non-human animal. Alternatively, a non-human homologue of the human NOVX gene, such as a mouse NOVX gene, can be isolated based on hybridization to the human NOVX cDNA (described further supra) and used as a transgene. Intronic sequences and polyadenylation signals can also be included in the transgene to increase the efficiency of expression of the transgene. A tissue-specific regulatory sequence(s) can be operably-linked to the NOVX transgene to direct expression of NOVX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the NOVX transgene in its genome and/or expression of NOVX mRNA in tissues or cells of the animals. A transgenic founder animal can then be used to breed additional animals carrying the transgene. Moreover, transgenic animals carrying a transgene-encoding NOVX protein can further be bred to other transgenic animals carrying other transgenes.

To create a homologous recombinant animal, a vector is prepared which contains at least a portion of an NOVX gene into which a deletion, addition or substitution has been introduced to thereby alter, e.g., functionally disrupt, the NOVX gene. The NOVX gene can be a human gene (e.g., the cDNA of SEQ ID NOS:1, 3, 4, 6, 8, 10, 12, 14, 16, 18, 20, 22, 24, and 26), but more preferably, is a non-human homologue of a human NOVX gene. For example, a mouse homologue of human NOVX gene of SEQ ID NOS:1, 3, 4, 6, 8, 10, 12, 14, 16, 18, 20, 22, 24, and 26 can be used to construct a homologous recombination vector suitable for altering an endogenous NOVX gene in the mouse genome. In one embodiment, the vector is designed such that, upon homologous recombination, the endogenous NOVX gene is functionally disrupted (i.e., no longer encodes a functional protein; also referred to as a “knock out” vector).

Alternatively, the vector can be designed such that, upon homologous recombination, the endogenous NOVX gene is mutated or otherwise altered but still encodes functional protein (e.g., the upstream regulatory region can be altered to thereby alter the expression of the endogenous NOVX protein). In the homologous recombination vector, the altered portion of the NOVX gene is flanked at its 5′- and 3′-termini by additional nucleic acid of the NOVX gene to allow for homologous recombination to occur between the exogenous NOVX gene carried by the vector and an endogenous NOVX gene in an embryonic stem cell. The additional flanking NOVX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced NOVX gene has homologously-recombined with the endogenous NOVX gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter Go phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The NOVX nucleic acid molecules, NOVX proteins, and anti-NOVX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an NOVX protein or anti-NOVX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express NOVX protein (e.g., via a recombinant expression vector in a host cell in gene therapy applications), to detect NOVX mRNA (e.g., in a biological sample) or a genetic lesion in an NOVX gene, and to modulate NOVX activity, as described further, below. In addition, the NOVX proteins can be used to screen drugs or compounds that modulate the NOVX protein activity or expression as well as to treat disorders characterized by insufficient or excessive production of NOVX protein or production of NOVX protein forms that have decreased or aberrant activity compared to NOVX wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease(possesses anti-microbial activity) and the various dyslipidemias. In addition, the anti-NOVX antibodies of the invention can be used to detect and isolate NOVX proteins and modulate NOVX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to NOVX proteins or have a stimulatory or inhibitory effect on, e.g., NOVX protein expression or NOVX protein activity. The invention also includes compounds identified in the screening assays described herein.

In one embodiment, the invention provides assays for screening candidate or test compounds which bind to or modulate the activity of the membrane-bound form of an NOVX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12:145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37:1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No.5,233,409.).

In one embodiment, an assay is a cell-based assay in which a cell which expresses a membrane-bound form of NOVX protein, or a biologically-active portion thereof, on the cell surface is contacted with a test compound and the ability of the test compound to bind to an NOVX protein determined. The cell, for example, can of mammalian origin or a yeast cell. Determining the ability of the test compound to bind to the NOVX protein can be accomplished, for example, by coupling the test compound with a radioisotope or enzymatic label such that binding of the test compound to the NOVX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of NOVX protein, or a biologically-active portion thereof, on the cell surface with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX protein or a biologically-active portion thereof as compared to the known compound.

In another embodiment, an assay is a cell-based assay comprising contacting a cell expressing a membrane-bound form of NOVX protein, or a biologically-active portion thereof, on the cell surface with a test compound and determining the ability of the test compound to modulate (e.g., stimulate or inhibit) the activity of the NOVX protein or biologically-active portion thereof. Determining the ability of the test compound to modulate the activity of NOVX or a biologically-active portion thereof can be accomplished, for example, by determining the ability of the NOVX protein to bind to or interact with an NOVX target molecule. As used herein, a “target molecule” is a molecule with which an NOVX protein binds or interacts in nature, for example, a molecule on the surface of a cell which expresses an NOVX interacting protein, a molecule on the surface of a second cell, a molecule in the extracellular milieu, a molecule associated with the internal surface of a cell membrane or a cytoplasmic molecule. An NOVX target molecule can be a non-NOVX molecule or an NOVX protein or polypeptide of the invention. In one embodiment, an NOVX target molecule is a component of a signal transduction pathway that facilitates transduction of an extracellular signal (e.g. a signal generated by binding of a compound to a membrane-bound NOVX molecule) through the cell membrane and into the cell. The target, for example, can be a second intercellular protein that has catalytic activity or a protein that facilitates the association of downstream signaling molecules with NOVX.

Determining the ability of the NOVX protein to bind to or interact with an NOVX target molecule can be accomplished by one of the methods described above for determining direct binding. In one embodiment, determining the ability of the NOVX protein to bind to or interact with an NOVX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising an NOVX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting an NOVX protein or biologically-active portion thereof with a test compound and determining the ability of the test compound to bind to the NOVX protein or biologically-active portion thereof. Binding of the test compound to the NOVX protein can be determined either directly or indirectly as described above. In one such embodiment, the assay comprises contacting the NOVX protein or biologically-active portion thereof with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX or biologically-active portion thereof as compared to the known compound.

In still another embodiment, an assay is a cell-free assay comprising contacting NOVX protein or biologically-active portion thereof with a test compound and determining the ability of the test compound to modulate (e.g. stimulate or inhibit) the activity of the NOVX protein or biologically-active portion thereof. Determining the ability of the test compound to modulate the activity of NOVX can be accomplished, for example, by determining the ability of the NOVX protein to bind to an NOVX target molecule by one of the methods described above for determining direct binding. In an alternative embodiment, determining the ability of the test compound to modulate the activity of NOVX protein can be accomplished by determining the ability of the NOVX protein further modulate an NOVX target molecule. For example, the catalytic/enzymatic activity of the target molecule on an appropriate substrate can be determined as described, supra.

In yet another embodiment, the cell-free assay comprises contacting the NOVX protein or biologically-active portion thereof with a known compound which binds NOVX protein to form an assay mixture, contacting the assay mixture with a test compound, and determining the ability of the test compound to interact with an NOVX protein, wherein determining the ability of the test compound to interact with an NOVX protein comprises determining the ability of the NOVX protein to preferentially bind to or modulate the activity of an NOVX target molecule.

The cell-free assays of the invention are amenable to use of both the soluble form or the membrane-bound form of NOVX protein. In the case of cell-free assays comprising the membrane-bound form of NOVX protein, it may be desirable to utilize a solubilizing agent such that the membrane-bound form of NOVX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl-N,N-dimethyl-3-ammonio-1-propane sulfonate, 3-(3-cholamidopropyl)dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either NOVX protein or its target molecule to facilitate separation of complexed from uncomplexed forms of one or both of the proteins, as well as to accommodate automation of the assay. Binding of a test compound to NOVX protein, or interaction of NOVX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-NOVX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or NOVX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of NOVX protein binding or activity determined using standard techniques.

Other techniques for immobilizing proteins on matrices can also be used in the screening assays of the invention. For example, either the NOVX protein or its target molecule can be immobilized utilizing conjugation of biotin and streptayidin. Biotinylated NOVX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with NOVX protein or target molecules, but which do not interfere with binding of the NOVX protein to its target molecule, can be derivatized to the wells of the plate, and unbound target or NOVX protein trapped in the wells by antibody conjugation. Methods for detecting such complexes, in addition to those described above for the GST-immobilized complexes, include immunodetection of complexes using antibodies reactive with the NOVX protein or target molecule, as well as enzyme-linked assays that rely on detecting an enzymatic activity associated with the NOVX protein or target molecule.

In another embodiment, modulators of NOVX protein expression are identified in a method wherein a cell is contacted with a candidate compound and the expression of NOVX mRNA or protein in the cell is determined. The level of expression of NOVX mRNA or protein in the presence of the candidate compound is compared to the level of expression of NOVX mRNA or protein in the absence of the candidate compound. The candidate compound can then be identified as a modulator of NOVX mRNA or protein expression based upon this comparison. For example, when expression of NOVX MRNA or protein is greater (i.e., statistically significantly greater) in the presence of the candidate compound than in its absence, the candidate compound is identified as a stimulator of NOVX mRNA or protein expression. Alternatively, when expression of NOVX mRNA or protein is less (statistically significantly less) in the presence of the candidate compound than in its absence, the candidate compound is identified as an inhibitor of NOVX mRNA or protein expression. The level of NOVX mRNA or protein expression in the cells can be determined by methods described herein for detecting NOVX mRNA or protein.

In yet another aspect of the invention, the NOVX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with NOVX (“NOVX-binding proteins” or “NOVX-bp”) and modulate NOVX activity. Such NOVX-binding proteins are also likely to be involved in the propagation of signals by the NOVX proteins as, for example, upstream or downstream elements of the NOVX pathway.

The two-hybrid system is based on the modular nature of most transcription factors, which consist of separable DNA-binding and activation domains. Briefly, the assay utilizes two different DNA constructs. In one construct, the gene that codes for NOVX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an NOVX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with NOVX.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of the NOVX sequences, SEQ ID NOS: 1, 3, 4, 6, 8, 10, 12, 14, 16, 18, 20, 22, 24, and 26, or fragments or derivatives thereof, can be used to map the location of the NOVX genes, respectively, on a chromosome. The mapping of the NOVX sequences to chromosomes is an important first step in correlating these sequences with genes associated with disease.

Briefly, NOVX genes can be mapped to chromosomes by preparing PCR primers (preferably 15-25 bp in length) from the NOVX sequences. Computer analysis of the NOVX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the NOVX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the NOVX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the NOVX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The NOVX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the NOVX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The NOVX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If predicted coding sequences, such as those in SEQ ID NOS:1, 3, 4, 6, 8, 10, 12, 14, 16, 18, 20, 22, 24, and 26 are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining NOVX protein and/or nucleic acid expression as well as NOVX activity, in the context of a biological sample (e.g., blood, serum, cells, tissue) to thereby determine whether an individual is afflicted with a disease or disorder, or is at risk of developing a disorder, associated with aberrant NOVX expression or activity. The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers. The invention also provides for prognostic (or predictive) assays for determining whether an individual is at risk of developing a disorder associated with NOVX protein, nucleic acid expression or activity. For example, mutations in an NOVX gene can be assayed in a biological sample. Such assays can be used for prognostic or predictive purpose to thereby prophylactically treat an individual prior to the onset of a disorder characterized by or associated with NOVX protein, nucleic acid expression, or biological activity.

Another aspect of the invention provides methods for determining NOVX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of NOVX in clinical trials.

These and other agents are described in further detail in the following sections.

Diagnostic Assays

An exemplary method for detecting the presence or absence of NOVX in a biological sample involves obtaining a biological sample from a test subject and contacting the biological sample with a compound or an agent capable of detecting NOVX protein or nucleic acid (e.g., mRNA, genomic DNA) that encodes NOVX protein such that the presence of NOVX is detected in the biological sample. An agent for detecting NOVX mRNA or genomic DNA is a labeled nucleic acid probe capable of hybridizing to NOVX mRNA or genomic DNA. The nucleic acid probe can be, for example, a full-length NOVX nucleic acid, such as the nucleic acid of SEQ ID NOS:1, 3, 4, 6, 8, 10, 12, 14, 16, 18, 20, 22, 24, and 26, or a portion thereof, such as an oligonucleotide of at least 15, 30, 50, 100, 250 or 500 nucleotides in length and sufficient to specifically hybridize under stringent conditions to NOVX mRNA or genomic DNA. Other suitable probes for use in the diagnostic assays of the invention are described herein.

An agent for detecting NOVX protein is an antibody capable of binding to NOVX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect NOVX mRNA, protein, or genomic DNA in a biological sample in vitro as well as in vivo. For example, in vitro techniques for detection of NOVX mRNA include Northern hybridizations and in situ hybridizations. In vitro techniques for detection of NOVX protein include enzyme linked immunosorbent assays (ELISAs), Western blots, immunoprecipitations, and immunofluorescence. In vitro techniques for detection of NOVX genomic DNA include Southern hybridizations. Furthermore, in vivo techniques for detection of NOVX protein include introducing into a subject a labeled anti-NOVX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting NOVX protein, mRNA, or genomic DNA, such that the presence of NOVX protein, mRNA or genomic DNA is detected in the biological sample, and comparing the presence of NOVX protein, mRNA or genomic DNA in the control sample with the presence of NOVX protein, mRNA or genomic DNA in the test sample.

The invention also encompasses kits for detecting the presence of NOVX in a biological sample. For example, the kit can comprise: a labeled compound or agent capable of detecting NOVX protein or mRNA in a biological sample; means for determining the amount of NOVX in the sample; and means for comparing the amount of NOVX in the sample with a standard. The compound or agent can be packaged in a suitable container. The kit can further comprise instructions for using the kit to detect NOVX protein or nucleic acid.

Prognostic Assays

The diagnostic methods described herein can furthermore be utilized to identify subjects having or at risk of developing a disease or disorder associated with aberrant NOVX expression or activity. For example, the assays described herein, such as the preceding diagnostic assays or the following assays, can be utilized to identify a subject having or at risk of developing a disorder associated with NOVX protein, nucleic acid expression or activity. Alternatively, the prognostic assays can be utilized to identify a subject having or at risk for developing a disease or disorder. Thus, the invention provides a method for identifying a disease or disorder associated with aberrant NOVX expression or activity in which a test sample is obtained from a subject and NOVX protein or nucleic acid (e.g., mRNA, genomic DNA) is detected, wherein the presence of NOVX protein or nucleic acid is diagnostic for a subject having or at risk of developing a disease or disorder associated with aberrant NOVX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant NOVX expression or activity. For example, such methods can be used to determine whether a subject can be effectively treated with an agent for a disorder. Thus, the invention provides methods for determining whether a subject can be effectively treated with an agent for a disorder associated with aberrant NOVX expression or activity in which a test sample is obtained and NOVX protein or nucleic acid is detected (e.g., wherein the presence of NOVX protein or nucleic acid is diagnostic for a subject that can be administered the agent to treat a disorder associated with aberrant NOVX expression or activity).

The methods of the invention can also be used to detect genetic lesions in an NOVX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding an NOVX-protein, or the misexpression of the NOVX gene. For example, such genetic lesions can be detected by ascertaining the existence of at least one of: (i) a deletion of one or more nucleotides from an NOVX gene; (ii) an addition of one or more nucleotides to an NOVX gene; (iii) a substitution of one or more nucleotides of an NOVX gene, (iv) a chromosomal rearrangement of an NOVX gene; (v) an alteration in the level of a messenger RNA transcript of an NOVX gene, (vi) aberrant modification of an NOVX gene, such as of the methylation pattern of the genomic DNA, (vii) the presence of a non-wild-type splicing pattern of a messenger RNA transcript of an NOVX gene, (viii) a non-wild-type level of an NOVX protein, (ix) allelic loss of an NOVX gene, and (x) inappropriate post-translational modification of an NOVX protein. As described herein, there are a large number of assay techniques known in the art which can be used for detecting lesions in an NOVX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the NOVX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to an NOVX gene under conditions such that hybridization and amplification of the NOVX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in an NOVX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in NOVX can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in NOVX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the NOVX gene and detect mutations by comparing the sequence of the sample NOVX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the NOVX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type NOVX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S₁ nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in NOVX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on an NOVX sequence, e.g., a wild-type NOVX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in NOVX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control NOVX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6:1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving an NOVX gene.

Furthermore, any cell type or tissue, preferably peripheral blood leukocytes, in which NOVX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on NOVX activity (e.g., NOVX gene expression), as identified by a screening assay described herein can be administered to individuals to treat (prophylactically or therapeutically) disorders (The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers.) In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of NOVX protein, expression of NOVX nucleic acid, or mutation content of NOVX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of NOVX protein, expression of NOVX nucleic acid, or mutation content of NOVX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with an NOVX modulator, such as a modulator identified by one of the exemplary screening assays described herein.

Monitoring of Effects During Clinical Trials

Monitoring the influence of agents (e.g., drugs, compounds) on the expression or activity of NOVX (e.g., the ability to modulate aberrant cell proliferation and/or differentiation) can be applied not only in basic drug screening, but also in clinical trials. For example, the effectiveness of an agent determined by a screening assay as described herein to increase NOVX gene expression, protein levels, or upregulate NOVX activity, can be monitored in clinical trails of subjects exhibiting decreased NOVX gene expression, protein levels, or downregulated NOVX activity. Alternatively, the effectiveness of an agent determined by a screening assay to decrease NOVX gene expression, protein levels, or downregulate NOVX activity, can be monitored in clinical trails of subjects exhibiting increased NOVX gene expression, protein levels, or upregulated NOVX activity. In such clinical trials, the expression or activity of NOVX and, preferably, other genes that have been implicated in, for example, a cellular proliferation or immune disorder can be used as a “read out” or markers of the immune responsiveness of a particular cell.

By way of example, and not of limitation, genes, including NOVX, that are modulated in cells by treatment with an agent (e.g., compound, drug or small molecule) that modulates NOVX activity (e.g., identified in a screening assay as described herein) can be identified. Thus, to study the effect of agents on cellular proliferation disorders, for example, in a clinical trial, cells can be isolated and RNA prepared and analyzed for the levels of expression of NOVX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of NOVX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of an NOVX protein, mRNA, or genomic DNA in the preadministration sample; (iii) obtaining one or more post-administration samples from the subject; (iv) detecting the level of expression or activity of the NOVX protein, mRNA, or genomic DNA in the post-administration samples; (v) comparing the level of expression or activity of the NOVX protein, mRNA, or genomic DNA in the pre-administration sample with the NOVX protein, mRNA, or genomic DNA in the post administration sample or samples; and (vi) altering the administration of the agent to the subject accordingly. For example, increased administration of the agent may be desirable to increase the expression or activity of NOVX to higher levels than detected, i.e., to increase the effectiveness of the agent. Alternatively, decreased administration of the agent may be desirable to decrease expression or activity of NOVX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant NOVX expression or activity. The disorders include cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, prostate cancer, neoplasm; adenocarcinoma, lymphoma, uterus cancer, fertility, hemophilia, hypercoagulation, idiopathic thrombocytopenic purpura, immunodeficiencies, graft versus host disease, AIDS, bronchial asthma, Crohn's disease; multiple sclerosis, treatment of Albright Hereditary Ostoeodystrophy, and other diseases, disorders and conditions of the like.

These methods of treatment will be discussed more fully, below.

Disease and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (ie., due to a heterologous insertion within the coding sequences of coding sequences to an aforementioned peptide) that are utilized to “knockout” endogenous function of an aforementioned peptide by homologous recombination (see, e.g., Capecchi, 1989. Science 244: 1288-1292); or (v) modulators (i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant NOVX expression or activity, by administering to the subject an agent that modulates NOVX expression or at least one NOVX activity. Subjects at risk for a disease that is caused or contributed to by aberrant NOVX expression or activity can be identified by, for example, any or a combination of diagnostic or prognostic assays as described herein. Administration of a prophylactic agent can occur prior to the manifestation of symptoms characteristic of the NOVX aberrancy, such that a disease or disorder is prevented or, alternatively, delayed in its progression. Depending upon the type of NOVX aberrancy, for example, an NOVX agonist or NOVX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating NOVX expression or activity for therapeutic purposes. The modulatory method of the invention involves contacting a cell with an agent that modulates one or more of the activities of NOVX protein activity associated with the cell. An agent that modulates NOVX protein activity can be an agent as described herein, such as a nucleic acid or a protein, a naturally-occurring cognate ligand of an NOVX protein, a peptide, an NOVX peptidomimetic, or other small molecule. In one embodiment, the agent stimulates one or more NOVX protein activity. Examples of such stimulatory agents include active NOVX protein and a nucleic acid molecule encoding NOVX that has been introduced into the cell. In another embodiment, the agent inhibits one or more NOVX protein activity. Examples of such inhibitory agents include antisense NOVX nucleic acid molecules and anti-NOVX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of an NOVX protein or nucleic acid molecule. In one embodiment, the method involves administering an agent (e.g., an agent identified by a screening assay described herein), or combination of agents that modulates (e.g., up-regulates or down-regulates) NOVX expression or activity. In another embodiment, the method involves administering an NOVX protein or nucleic acid molecule as therapy to compensate for reduced or aberrant NOVX expression or activity.

Stimulation of NOVX activity is desirable in situations in which NOVX is abnormally downregulated and/or in which increased NOVX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention The NOVX nucleic acids and proteins of the invention are useful in potential prophylactic and therapeutic applications implicated in a variety of disorders including, but not limited to: metabolic disorders, diabetes, obesity, infectious disease, anorexia, cancer-associated cancer, neurodegenerative disorders, Alzheimer's Disease, Parkinson's Disorder, immune disorders, hematopoietic disorders, and the various dyslipidemias, metabolic disturbances associated with obesity, the metabolic syndrome X and wasting disorders associated with chronic diseases and various cancers.

As an example, a cDNA encoding the NOVX protein of the invention may be useful in gene therapy, and the protein may be useful when administered to a subject in need thereof. By way of non-limiting example, the compositions of the invention will have efficacy for treatment of patients suffering from: metabolic disorders, diabetes, obesity, infectious disease, anorexia, cancer-associated cachexia, cancer, neurodegenerative disorders, Alzheimer's Disease, Parkinson's Disorder, immune disorders, hematopoietic disorders, and the various dyslipidemias.

Both the novel nucleic acid encoding the NOVX protein, and the NOVX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies, which immunospecifically-bind to the novel substances of the invention for use in therapeutic or diagnostic methods.

The invention will be further described in the following examples, which do not limit the scope of the invention described in the claims.

### EXAMPLES

### Example 1

### Identification of NOVX Clones

The novel NOVX target sequences identified in the present invention were subjected to the exon linking process to confirm the sequence. PCR primers were designed by starting at the most upstream sequence available, for the forward primer, and at the most downstream sequence available for the reverse primer. Table 11A shows the sequences of the PCR primers used for obtaining different clones. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The PCR product derived from exon linking was cloned into the pCR2.1 vector from Invitrogen. The resulting bacterial clone has an insert covering the entire open reading frame cloned into the pCR2.1 vector. Table 17B shows a list of these bacterial clones. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported herein.

TABLE 11A

PCR Primers for Exon Linking

| | | SEQ | | SEQ |
|---|---|---|---|---|
| NOVX | | ID | | ID |
| Clone | Primer 1 (5′-3′) | NO | Primer 2 (5′-3′) | NO |
| NOV1C | TCATCACATGACAACATGAAGCTGT | 87 | GAAAGCCCTCAAACTCTCCATCTATG | 88 |
| NOV7a | CCAATCTCTGATGCCCTGCGAT | 89 | AGGTCAGTGCCGGAGCCTCC | 90 |

Physical clone: Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

TABLE 11B
Physical Clones for PCR products

| NOVX Clone | Bacterial Clone |
|---|---|
| NOV1 | Physical clone: 128940::83420733.698715.E24 |
| NOV2 | Physical clone: AL357059, AL022344, AL3555530, |
| | AL356100, AC016042 |
| NOV4 | Physical clone: AC009785 |
| NOV5 | Genomic clone: GMChromosome4 |
| NOV7a | Genomic file: gb_AC010319 HTG Homo sapiens\| |
| | chromosome 19 CTD-2521M24 |
| NOV8 | Physical clone: AC008803, AC010449, AC026718 |

### Example 2

### Quantitative Expression Analysis of Clones in Various Cells and Tissues

The quantitative expression of various clones was assessed using microtiter plates containing RNA samples from a variety of normal and pathology-derived cells, cell lines and tissues using real time quantitative PCR (RTQ PCR). RTQ PCR was performed on a Perkin-Elmer Biosystems ABI PRISM® 7700 Sequence Detection System. Various collections of samples are assembled on the plates, and referred to as Panel 1 (containing normal tissues and cancer cell lines), Panel 2 (containing samples derived from tissues from normal and cancer sources), Panel 3 (containing cancer cell lines), Panel 4 (containing cells and cell lines from normal tissues and cells related to inflammatory conditions), AI_comprehensive_panel (containing normal tissue and samples from autoinflammatory diseases), Panel CNSD.01 (containing samples from normal and diseased brains) and CNS_neurodegeneration_panel (containing samples from normal and diseased brains).

First, the RNA samples were normalized to reference nucleic acids such as constitutively expressed genes (for example, β-actin and GAPDH). Normalized RNA (5 ul) was converted to cDNA and analyzed by RTQ-PCR using One Step RT-PCR Master Mix Reagents (PE Biosystems; Catalog No. 4309169) and gene-specific primers according to the manufacturer's instructions. Probes and primers were designed for each assay according to Perkin Elmer Biosystem's Primer Express Software package (version I for Apple Computer's Macintosh Power PC) or a similar algorithm using the target sequence as input. Default settings were used for reaction conditions and the following parameters were set before selecting primers: primer concentration=250 nM, primer melting temperature (Tₘ) range=58°-60° C., primer optimal Tm=59° C., maximum primer difference=2° C., probe does not have 5′ G, probe Tₘ must be 10° C. greater than primer Tₘ, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200 nM.

PCR conditions: Normalized RNA from each tissue and each cell line was spotted in each well of a 96 well PCR plate (Perkin Elmer Biosystems). PCR cocktails including two probes (a probe specific for the target clone and another gene-specific probe multiplexed with the target probe) were set up using 1× TaqMan™ PCR Master Mix for the PE Biosystems 7700, with 5 mM MgCl2, dNTPs (dA, G, C, U at 1:1:1:2 ratios), 0.25 U/ml AmpliTaq Gold™ (PE Biosystems), and 0.4 U/μl RNase inhibitor, and 0.25 U/μl reverse transcriptase. Reverse transcription was performed at 48° C. for 30 minutes followed by amplification/PCR cycles as follows: 95° C. 10 min, then 40 cycles of 95° C. for 15 seconds, 60° C. for 1 minute. Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100.

Panel 1

In the results for Panel 1, the following abbreviations are used:

ca.=carcinoma,

*=established from metastasis,

met=metastasis,

s cell var=small cell variant,

non-s=non-sm=non-small,

squam=squamous,

pl. eff=pl effusion=pleural effusion,

glio=glioma,

astro=astrocytoma, and

neuro=neuroblastoma.

Panel 2

The plates for Panel 2 generally include 2 control wells and 94 test samples composed of RNA or cDNA isolated from human tissue procured by surgeons working in close cooperation with the National Cancer Institute's Cooperative Human Tissue Network (CHTN) or the National Disease Research Initiative (NDRI). The tissues are derived from human malignancies and in cases where indicated many malignant tissues have “matched margins” obtained from noncancerous tissue just adjacent to the tumor. These are termed normal adjacent tissues and are denoted “NAT” in the results below. The tumor tissue and the “matched margins” are evaluated by two independent pathologists (the surgical pathologists and again by a pathologists at NDRI or CHTN). This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue, in Table RR). In addition, RNA and cDNA samples were obtained from various human tissues derived from autopsies performed on elderly people or sudden death victims (accidents, etc.). These tissues were ascertained to be free of disease and were purchased from various commercial sources such as Clontech (Palo Alto, Calif.), Research Genetics, and Invitrogen.

RNA integrity from all samples is controlled for quality by visual assessment of agarose gel electropherograms using 28S and 18S ribosomal RNA staining intensity ratio as a guide (2:1 to 2.5:1 28s:18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

Panel 3D

The plates of Panel 3D are comprised of 94 cDNA samples and two control samples. Specifically, 92 of these samples are derived from cultured human cancer cell lines, 2 samples of human primary cerebellar tissue and 2 controls. The human cell lines are generally obtained from ATCC (American Type Culture Collection), NCI or the German tumor cell bank and fall into the following tissue groups: Squamous cell carcinoma of the tongue, breast cancer, prostate cancer, melanoma, epidermoid carcinoma, sarcomas, bladder carcinomas, pancreatic cancers, kidney cancers, leukemias/lymphomas, ovarian/uterine/cervical, gastric, colon, lung and CNS cancer cell lines. In addition, there are two independent samples of cerebellum. These cells are all cultured under standard recommended conditions and RNA extracted using the standard procedures. The cell lines in panel 3D and 1.3D are of the most common cell lines used in the scientific literature.

RNA integrity from all samples is controlled for quality by visual assessment of agarose gel electropherograms using 28S and 18S ribosomal RNA staining intensity ratio as a guide (2:1 to 2.5:1 28s:18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

Panel 4

Panel 4 includes samples on a 96 well plate (2 control wells, 94 test samples) composed of RNA (Panel 4r) or cDNA (Panel 4d) isolated from various human cell lines or tissues related to inflammatory conditions. Total RNA from control normal tissues such as colon and lung (Stratagene ,La Jolla, Calif.) and thymus and kidney (Clontech) were employed. Total RNA from liver tissue from cirrhosis patients and kidney from lupus patients was obtained from BioChain (Biochain Institute, Inc., Hayward, Calif.). Intestinal tissue for RNA preparation from patients diagnosed as having Crohn's disease and ulcerative colitis was obtained from the National Disease Research Interchange (NDRI) (Philadelphia, Pa.).

Astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, human umbilical vein endothelial cells were all purchased from Clonetics (Walkersville, Md.) and grown in the media supplied for these cell types by Clonetics. These primary cell types were activated with various cytokines or combinations of cytokines for 6 and/or 12-14 hours, as indicated. The following cytokines were used; IL-1 beta at approximately 1-5 ng/ml, TNF alpha at approximately 5-10 ng/ml, IFN gamma at approximately 20-50 ng/ml, IL-4 at approximately 5-10 ng/ml, IL-9 at approximately 5-10 ng/ml, IL-13 at approximately 5-10 ng/ml. Endothelial cells were sometimes starved for various times by culture in the basal media from Clonetics with 0.1% serum.

Mononuclear cells were prepared from blood of employees at CuraGen Corporation, using Ficoll. LAK cells were prepared from these cells by culture in DMEM 5% FCS (Hyclone), 100 μM non essential amino acids (Gibco/Life Technologies, Rockville, Md.), 1 mM sodium pyruvate (Gibco), mercaptoethanol 5.5×10⁻⁵ M (Gibco), and 10 mM Hepes (Gibco) and Interleukin 2 for 4-6 days. Cells were then either activated with 10-20 ng/ml PMA and 1-2 μg/ml ionomycin, IL-12 at 5-10 ng/ml, IFN gamma at 20-50 ng/ml and IL-18 at 5-10 ng/ml for 6 hours. In some cases, mononuclear cells were cultured for 4-5 days in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) with PHA (phytohemagglutinin) or PWM (pokeweed mitogen) at approximately 5 μg/ml. Samples were taken at 24, 48 and 72 hours for RNA preparation. MLR (mixed lymphocyte reaction) samples were obtained by taking blood from two donors, isolating the mononuclear cells using Ficoll and mixing the isolated mononuclear cells 1:1 at a final concentration of approximately 2×10⁶ cells/ml in DMEM 5% FCS (Hyclone), 100 μM non essential amino acids (Gibco), 1 mM sodium pyruvate (Gibco), mercaptoethanol (5.5×10⁻⁵ M) (Gibco), and 10 mM Hepes (Gibco). The MLR was cultured and samples taken at various time points ranging from 1-7 days for RNA preparation.

Monocytes were isolated from mononuclear cells using CD14 Miltenyi Beads, +ve VS selection columns and a Vario Magnet according to the manufacturer's instructions. Monocytes were differentiated into dendritic cells by culture in DMEM 5% fetal calf serum (FCS) (Hyclone, Logan, Utah), 100 μM non essential amino acids (Gibco), 1 mM sodium pyruvate (Gibco), mercaptoethanol 5.5×10⁻⁵ M (Gibco), and 10 mM Hepes (Gibco), 50 ng/ml GMCSF and 5 ng/ml IL-4 for 5-7 days. Macrophages were prepared by culture of monocytes for 5-7 days in DMEM 5% FCS (Hyclone), 100 lM non essential amino acids (Gibco), 1 mM sodium pyruvate (Gibco), mercaptoethanol 5.5×10⁻⁵ M (Gibco), 10 mM Hepes (Gibco) and 10% AB Human Serum or MCSF at approximately 50 ng/ml. Monocytes, macrophages and dendritic cells were stimulated for 6 and 12-14 hours with lipopolysaccharide (LPS) at 100 ng/ml. Dendritic cells were also stimulated with anti-CD40 monoclonal antibody (Pharmingen) at 10 μg/ml for 6 and 12-14 hours.

CD4 lymphocytes, CD8 lymphocytes and NK cells were also isolated from mononuclear cells using CD4, CD8 and CD56 Miltenyi beads, positive VS selection columns and a Vario Magnet according to the manufacturer's instructions. CD45RA and CD45RO CD4 lymphocytes were isolated by depleting mononuclear cells of CD8, CD56, CD14 and CD19 cells using CD8, CD56, CD14 and CD19 Miltenyi beads and positive selection. Then CD45RO beads were used to isolate the CD45RO CD4 lymphocytes with the remaining cells being CD45RA CD4 lymphocytes. CD45RA CD4, CD45RO CD4 and CD8 lymphocytes were placed in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and plated at 10⁶ cells/ml onto Falcon 6 well tissue culture plates that had been coated overnight with 0.5 μg/ml anti-CD28 (Pharmingen) and 3 ug/ml anti-CD3 (OKT3, ATCC) in PBS. After 6 and 24 hours, the cells were harvested for RNA preparation. To prepare chronically activated CD8 lymphocytes, we activated the isolated CD8 lymphocytes for 4 days on anti-CD28 and anti-CD3 coated plates and then harvested the cells and expanded them in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and IL-2. The expanded CD8 cells were then activated again with plate bound anti-CD3 and anti-CD28 for 4 days and expanded as before. RNA was isolated 6 and 24 hours after the second activation and after 4 days of the second expansion culture. The isolated NK cells were cultured in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and IL-2 for 4-6 days before RNA was prepared.

To obtain B cells, tonsils were procured from NDRI. The tonsil was cut up with sterile dissecting scissors and then passed through a sieve. Tonsil cells were then spun down and resupended at 10⁶ cells/ml in DMEM 5% FCS (kyclone), 100 μM non essential amino acids (Gibco), 1 mM sodium pyruvate (Gibco), mercaptoethanol 5.5×10⁻⁵ M (Gibco), and 10 mM Hepes (Gibco). To activate the cells, we used PWM at 5 μg/ml or anti-CD40 (Pharmingen) at approximately 10 μg/ml and IL-4 at 5-10 ng/ml. Cells were harvested for RNA preparation at 24,48 and 72 hours.

To prepare the primary and secondary Th1/Th2 and Tr1 cells, six-well Falcon plates were coated overnight with 10 μg/ml anti-CD28 (Pharmingen) and 2 μg/ml OKT3 (ATCC), and then washed twice with PBS. Umbilical cord blood CD4 lymphocytes (Poietic Systems, German Town, Md.) were cultured at 10⁵-10⁶ cells/ml in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and IL-2 (4 ng/ml). IL-12 (5 ng/ml) and anti-IL4 (1 □g/ml) were used to direct to Th1, while IL4 (5 ng/ml) and anti-IFN gamma (1 □g/ml) were used to direct to Th2 and IL-10 at 5 ng/ml was used to direct to Tr1. After 4-5 days, the activated Th1, Th2 and Tr1 lymphocytes were washed once in DMEM and expanded for 4-7 days in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and IL-2 (1 ng/ml). Following this, the activated Th1, Th2 and Tr1 lymphocytes were re-stimulated for 5 days with anti-CD28/OKT3 and cytokines as described above, but with the addition of anti-CD95L (1 □g/ml) to prevent apoptosis. After 4-5 days, the Th1, Th2 and Tr1 lymphocytes were washed and then expanded again with IL-2 for 4-7 days. Activated Th1 and Th2 lymphocytes were maintained in this way for a maximum of three cycles. RNA was prepared from primary and secondary Th1, Th2 and Tr1 after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, EOL-1, KU-812. EOL cells were further differentiated by culture in 0.1 mM dbcAMP at 5×10⁵ cells/ml for 8 days, changing the media every 3 days and adjusting the cell concentration to 5×10⁵ cells/ml. For the culture of these cells, we used DMEM or RPMI (as recommended by the ATCC), with the addition of 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco). RNA was either prepared from resting cells or cells activated with PMA at 10 ng/ml and ionomycin at 1 μg/ml for 6 and 14 hours. Keratinocyte line CCD106 and an airway epithelial tumor line NCI-H292 were also obtained from the ATCC. Both were cultured in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco). CCD1106 cells were activated for 6 and 14 hours with approximately 5 ng/ml TNF alpha and 1 ng/ml IL-1 beta, while NCI-H292 cells were activated for 6 and 14 hours with the following cytokines: 5 ng/ml IL-4, 5 ng/ml IL-9, 5 ng/ml IL-13 and 25 ng/ml IFN gamma.

For these cell lines and blood cells, RNA was prepared by lysing approximately 10⁷ cells/ml using Trizol (Gibco BRL). Briefly, 1/10 volume of bromochloropropane (Molecular Research Corporation) was added to the RNA sample, vortexed and after 10 minutes at room temperature, the tubes were spun at 14,000 rpm in a Sorvall SS34 rotor. The aqueous phase was removed and placed in a 15 ml Falcon Tube. An equal volume of isopropanol was added and left at −20 degrees C. overnight. The precipitated RNA was spun down at 9,000 rpm for 15 min in a Sorvall SS34 rotor and washed in 70% ethanol. The pellet was redissolved in 300 μl of RNAse-free water and 35 μl buffer (Promega) 5 μl DTT, 7 μl RNAsin and 8 μl DNAse were added. The tube was incubated at 37 degrees C. for 30 minutes to remove contaminating genomic DNA, extracted once with phenol chloroform and re-precipitated with 1/10 volume of 3 M sodium acetate and 2 volumes of 100% ethanol. The RNA was spun down and placed in RNAse free water. RNA was stored at −80 degrees C.

Panel CNSD.01

The plates for Panel CNSD.01 include two control wells and 94 test samples comprised of cDNA isolated from postmortem human brain tissue obtained from the Harvard Brain Tissue Resource Center. Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains two brains from each of the following diagnoses: Alzheimer's disease, Parkinson's disease, Huntington's disease, Progressive Supernuclear Palsy, Depression, and “Normal controls”. Within each of these brains, the following regions are represented: cingulate gyrus, temporal pole, globus palladus, substantia nigra, Brodman Area 4 (primary motor strip), Brodman Area 7 (parietal cortex), Brodman Area 9 (prefrontal cortex), and Brodman area 17 (occipital cortex). Not all brain regions are represented in all cases; e.g., Huntington's disease is characterized in part by neurodegeneration in the globus palladus, thus this region is impossible to obtain from confirmed Huntington's cases. Likewise Parkinson's disease is characterized by degeneration of the substantia nigra making this region more difficult to obtain. Normal control brains were examined for neuropathology and found to be free of any pathology consistent with neurodegeneration.

RNA integrity from all samples is controlled for quality by visual assessment of agarose gel electropherograms using 28S and 18S ribosomal RNA staining intensity ratio as a guide (2:1 to 2.5:1 28s:18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

In the labels employed to identify tissues in the CNS panel, the following abbreviations are used:

PSP=Progressive supranuclear palsy

Sub Nigra=Substantia nigra

Glob Palladus=Globus palladus

Temp Pole=Temporal pole

Cing Gyr=Cingulate gyrus

BA 4=Brodman Area 4

Panel CNS_Neurodegeneration_V1.0

The plates for Panel CNS_Neurodegeneration_V1.0 include two control wells and 47 test samples comprised of cDNA isolated from postmortem human brain tissue obtained from the Harvard Brain Tissue Resource Center (McLean Hospital) and the Human Brain and Spinal Fluid Resource Center (VA Greater Los Angeles Healthcare System). Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains six brains from Alzheimer's disease (AD) pateins, and eight brains from “Normal controls” who showed no evidence of dementia prior to death. The eight normal control brains are divided into two categories: Controls with no dementia and no Alzheimer's like pathology (Controls) and controls with no dementia but evidence of severe Alzheimer's like pathology, (specifically senile plaque load rated as level 3 on a scale of 0-3; 0=no evidence of plaques, 3 =severe AD senile plaque load). Within each of these brains, the following regions are represented: Hippocampus, Temporal cortex (Broddmann Area 21), Somatosensory cortex (Broddmann area 7), and Occipital cortex (Brodmann area 17). These regions were chosen to encompass all levels of neurodegeneration in AD. The hippocampus is a region of early and severe neuronal loss in AD; the temporal cortex is known to show neurodegeneration in AD after the hippocampus; the somatosensory cortex shows moderate neuronal death in the late stages of the disease; the occipital cortex is spared in AD and therefore acts as a “control” region within AD patients. Not all brain regions are represented in all cases.

RNA integrity from all samples is controlled for quality by visual assessment of agarose gel electropherograms using 28S and 18S ribosomal RNA staining intensity ratio as a guide (2:1 to 2.5:1 28s:18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

In the labels employed to identify tissues in the CNS_Neurodegeneration_V1.0 panel, the following abbreviations are used:

AD=Alzheimer's disease brain; patient was demented and showed AD-like pathology upon autopsy

Control=Control brains; patient not demented, showing no neuropathology

Control (Path)=Control brains; pateint not demented but showing sever AD-like pathology

SupTemporal Ctx=Superior Temporal Cortex
Inf Temporal Ctx=Inferior Temporal Cortex

NOV1a

Expression of gene NOV1a was assessed using the primer-probe sets Ag273b and Ag1094, described in Tables 12 and 13. Results from RTQ-PCR runs are shown in Tables 14, 15, 16, 17, and 18.

TABLE 12

Probe Name Ag273b

| | | | | Start | SEQ ID |
|---|---|---|---|---|---|
| Primers | Sequences | TM | Length | Position | NO: |
| Forward | 5′-CGGCTTGACGATGCTTCAC-3′ | | 19 | 13 | 91 |
| Probe | FAM-5′- | | 32 | 37 | 92 |
| | TGACTTTTCTGGGCTTACCAATGCTATTTCAA- | | | | |
| | 3′-TAMRA | | | | |
| Reverse | 5′-GCACCTATCTCAATATCTGCAATATTG-3′ | | 27 | 85 | 93 |

TABLE 13

Probe Name Ag1094

| | | | | Start | SEQ ID |
|---|---|---|---|---|---|
| Primers | Sequences | TM | Length | Position | NO: |
| Forward | 5′-ATGGACTGGAAAACCTGGAA-3′ | 59.4 | 20 | 192 | 94 |
| Probe | FAM-5′- | 66.5 | 29 | 213 | 95 |
| | TCCTGCAAGCAGATAACAATTTTATCACA- | | | | |
| | 3′-TAMRA | | | | |
| Reverse | 5′-TGCTAAAGGCACTTGGTTCA-3′ | 59.5 | 20 | 247 | 96 |

TABLE 14
Panel 1

| | Relative | | Relative |
|---|---|---|---|
| | Expression(%) | | Expression(%) |
| | tm566f_{—} | | tm566f_{—} |
| Tissue Name | ag273b | Tissue Name | ag273b |
| Endothelial cells | 0.0 | Renal ca. 786-0 | 0.0 |
| Endothelial cells (treated) | 0.0 | Renal ca. A498 | 0.0 |
| Pancreas | 0.0 | Renal ca. RXF 393 | 0.0 |
| Pancreatic ca. CAPAN 2 | 0.0 | Renal ca. ACHN | 0.0 |
| Adrenal gland | 0.0 | Renal ca. UO-31 | 0.0 |
| Thyroid | 0.0 | Renal ca. TK-10 | 0.0 |
| Salavary gland | 12.9 | Liver | 0.0 |
| Pituitary gland | 0.0 | Liver (fetal) | 0.0 |
| Brain (fetal) | 0.0 | Liver ca. (hepatoblast) HepG2 | 0.0 |
| Brain (whole) | 0.2 | Lung | 0.5 |
| Brain (amygdala) | 0.0 | Lung (fetal) | 2.2 |
| Brain (cerebellum) | 1.6 | Lung ca. (small cell) LX-1 | 0.0 |
| Brain (hippocampus) | 0.0 | Lung ca. (small cell) NCI-H69 | 2.7 |
| Brain (substantia nigra) | 0.0 | Lung ca. (s.cell var.) SHP-77 | 44.1 |
| Brain (thalamus) | 2.9 | Lung ca. (large cell)NCI-H460 | 0.0 |
| Brain (hypothalamus) | 0.0 | Lung ca. (non-sm. cell) A549 | 0.0 |
| Spinal cord | 0.0 | Lung ca. (non-s.cell) NCI-H23 | 14.7 |
| CNS ca. (glio/astro) U87-MG | 0.0 | Lung ca (non-s.cell) HOP-62 | 12.2 |
| CNS ca. (glio/astro) U-118-MG | 0.0 | Lung ca. (non-s. cl) NCI-H522 | 0.2 |
| CNS ca. (astro) SW1783 | 0.0 | Lung ca. (squam.) SW 900 | 11.9 |
| CNS ca.* (neuro; met) SK-N- | 6.6 | Lung ca. (squam.) NCI-H596 | 2.5 |
| AS | | | |
| CNS ca. (astro) SF-539 | 0.0 | Mammary gland | 4.8 |
| CNS ca. (astro) SNB-75 | 10.2 | Breast ca.* (pl. effusion) MCF-7 | 0.4 |
| CNS ca. (glio) SNB-19 | 24.3 | Breast ca.* (pl.ef) MDA-MB- | 0.0 |
| | | 231 | |
| CNS ca. (glio) U251 | 4.2 | Breast ca.* (pl. effusion) T47D | 7.2 |
| CNS ca. (glio) SF-295 | 37.6 | Breast ca. BT-549 | 0.0 |
| Heart | 1.5 | Breast ca. MDA-N | 0.0 |
| Skeletal muscle | 0.0 | Ovary | 0.0 |
| Bone marrow | 0.0 | Ovarian ca. OVCAR-3 | 0.0 |
| Thymus | 0.4 | Ovarian ca. OVCAR-4 | 0.0 |
| Spleen | 0.0 | Ovarian ca. OVCAR-5 | 6.2 |
| Lymph node | 0.0 | Ovarian ca. OVCAR-8 | 0.0 |
| Colon (ascending) | 9.9 | Ovarian ca. IGROV-1 | 0.0 |
| Stomach | 0.4 | Ovarian ca.* (ascites) SK-OV-3 | 0.0 |
| Small intestine | 4.2 | Uterus | 0.0 |
| Colon ca. SW480 | 0.0 | Placenta | 0.8 |
| Colon ca.* (SW480 met)SW620 | 0.0 | Prostate | 3.6 |
| Colon ca. HT29 | 34.4 | Prostate ca.* (bone met)PC-3 | 100.0 |
| Colon ca. HCT-116 | 0.0 | Testis | 0.0 |
| Colon ca. CaCo-2 | 0.0 | Melanoma Hs688(A).T | 0.0 |
| Colon ca. HCT-15 | 0.0 | Melanoma* (met) Hs688(B).T | 0.0 |
| Colon ca. HCC-2998 | 0.0 | Melanoma UACC-62 | 0.3 |
| Gastric ca.* (liver met) NCI- | 1.3 | Melanoma M14 | 0.0 |
| N87 | | | |
| Bladder | 0.1 | Melanoma LOX IMVI | 0.0 |
| Trachea | 8.9 | Melanoma* (met) SK-MEL-5 | 0.0 |
| Kidney | 0.2 | Melanoma SK-MEL-28 | 0.2 |
| Kidney (fetal) | 1.3 | | |

TABLE 15
Panel 1.3D

| | Relative Expression(%) | |
|---|---|---|
| | 1.3Dtm2741f_{—} | 1.3Dtm2838f_{—} |
| Tissue Name | ag1094 | ag1094 |
| Liver adenocarcinoma | 10.0 | 9.1 |
| Pancreas | 0.2 | 0.1 |
| Pancreatic ca. CAPAN 2 | 0.0 | 0.0 |
| Adrenal gland | 0.0 | 0.0 |
| Thyroid | 0.2 | 0.2 |
| Salivary gland | 8.9 | 4.3 |
| Pituitary gland | 0.0 | 0.2 |
| Brain (fetal) | 0.1 | 0.0 |
| Brain (whole) | 0.8 | 0.6 |
| Brain (amygdala) | 0.2 | 0.1 |
| Brain (cerebellum) | 0.5 | 0.7 |
| Brain (hippocampus) | 0.4 | 0.2 |
| Brain (substantia nigra) | 0.0 | 0.0 |
| Brain (thalamus) | 1.1 | 1.0 |
| Cerebral Cortex | 0.2 | 0.1 |
| Spinal cord | 0.2 | 0.0 |
| CNS ca. (glio/astro) U87-MG | 0.0 | 0.2 |
| CNS ca. (glio/astro) U-118-MG | 1.0 | 0.8 |
| CNS ca. (astro) SW1783 | 1.1 | 0.9 |
| CNS ca.* (neuro; met) SK-N-AS | 26.4 | 26.8 |
| CNS ca. (astro) SF-539 | 0.0 | 0.0 |
| CNS ca. (astro) SNB-75 | 15.1 | 12.9 |
| CNS ca. (glio) SNB-19 | 38.2 | 21.0 |
| CNS ca. (glio) U251 | 3.3 | 3.7 |
| CNS ca. (glio) SF-295 | 38.4 | 36.9 |
| Heart (fetal) | 0.2 | 0.5 |
| Heart | 0.6 | 0.3 |
| Fetal Skeletal | 2.9 | 2.2 |
| Skeletal muscle | 0.0 | 0.0 |
| Bone marrow | 0.0 | 0.2 |
| Thymus | 0.4 | 0.1 |
| Spleen | 0.0 | 0.0 |
| Lymph node | 0.0 | 0.0 |
| Colorectal | 1.6 | 0.6 |
| Stomach | 1.6 | 1.6 |
| Small intestine | 4.2 | 3.7 |
| Colon ca. SW480 | 0.0 | 0.0 |
| Colon ca.* (SW480 met)SW620 | 0.4 | 0.1 |
| Colon ca. HT29 | 21.0 | 25.5 |
| Colon ca. HCT-116 | 0.0 | 0.0 |
| Colon ca. CaCo-2 | 0.0 | 0.0 |
| 83219 CC Well to Mod Diff | 0.0 | 0.0 |
| (ODO3866) | | |
| Colon ca. HCC-2998 | 0.0 | 0.0 |
| Gastric ca.* (liver met) NCI-N87 | 21.3 | 20.7 |
| Bladder | 0.1 | 0.0 |
| Trachea | 12.5 | 12.9 |
| Kidney | 0.0 | 0.0 |
| Kidney (fetal) | 0.9 | 0.6 |
| Renal ca. 786-0 | 0.0 | 0.0 |
| Renal ca. A498 | 2.0 | 1.5 |
| Renal ca. RXF 393 | 0.0 | 0.0 |
| Renal ca. ACHN | 0.0 | 0.0 |
| Renal ca. UO-31 | 0.0 | 0.0 |
| Renal ca. TK-10 | 0.0 | 0.0 |
| Liver | 0.4 | 0.6 |
| Liver (fetal) | 1.6 | 1.0 |
| Liver ca. (hepatoblast) HepG2 | 0.0 | 0.0 |
| Lung | 1.8 | 1.4 |
| Lung (fetal) | 11.7 | 7.5 |
| Lung ca. (small cell) LX-1 | 0.2 | 0.0 |
| Lung ca. (small cell) NCI-H69 | 2.4 | 3.1 |
| Lung ca. (s.cell var.) SHP-77 | 100.0 | 100.0 |
| Lung ca. (large cell)NCI-H460 | 0.0 | 0.0 |
| Lung ca. (non-sm. cell) A549 | 0.3 | 0.8 |
| Lung ca. (non-s.cell) NCI-H23 | 12.8 | 12.8 |
| Lung ca. (non-s.cell) HOP-62 | 3.9 | 3.7 |
| Lung ca. (non-s.cl) NCI-H522 | 0.1 | 0.0 |
| Lung ca. (squam.) SW 900 | 4.8 | 6.4 |
| Lung ca. (squam.) NCI-H596 | 1.3 | 0.9 |
| Mammary gland | 3.4 | 3.3 |
| Breast ca.* (pl. effusion) MGF-7 | 1.2 | 0.7 |
| Breast ca.* (pl.ef) MDA-MB-231 | 0.0 | 0.0 |
| Breast ca.* (pl. effusion) T47D | 3.2 | 3.0 |
| Breast ca. BT-549 | 2.1 | 1.7 |
| Breast ca. MDA-N | 0.0 | 0.0 |
| Ovary | 0.7 | 0.3 |
| Ovarian ca. OVCAR-3 | 0.4 | 0.3 |
| Ovarian ca. OVCAR-4 | 0.1 | 0.0 |
| Ovarian ca. OVCAR-5 | 8.5 | 6.2 |
| Ovarian ca. OVCAR-8 | 0.0 | 0.0 |
| Ovarian ca. IGROV-1 | 0.0 | 0.0 |
| Ovarian ca.* (ascites) SK-OV-3 | 0.3 | 0.2 |
| Uterus | 0.4 | 0.2 |
| Placenta | 1.0 | 1.3 |
| Prostate | 1.1 | 1.1 |
| Prostate ca.* (bone met)PC-3 | 13.2 | 13.9 |
| Testis | 0.3 | 0.4 |
| Melanoma Hs688(A).T | 0.0 | 0.0 |
| Melanoma* (met) Hs688(B).T | 0.0 | 0.0 |
| Melanoma UACC-62 | 0.0 | 0.0 |
| Melanoma M14 | 0.0 | 0.0 |
| Melanoma LOX IMVI | 0.0 | 0.0 |
| Melanoma* (met) SK-MEL-5 | 0.1 | 0.5 |
| Adipose | 0.5 | 0.4 |

TABLE 16
Panel 2D

| | Relative Expression(%) | |
|---|---|---|
| | 2Dtm2837f_{—} | 2dtm2940f_{—} |
| Tissue Name | ag1094 | ag1094 |
| Normal Colon GENPAK 061003 | 12.4 | 11.4 |
| 83219 CC Well to Mod Diff (ODO3866) | 0.0 | 0.0 |
| 83220 CC NAT (ODO3866) | 1.0 | 1.5 |
| 83221 CC Gr.2 rectosigmoid (ODO3868) | 0.3 | 0.0 |
| 83222 CC NAT (ODO3868) | 0.4 | 0.2 |
| 83235 CC Mod Diff (ODO3920) | 0.0 | 0.0 |
| 83236 CC NAT (ODO3920) | 0.8 | 0.8 |
| 83237 CC Gr.2 ascend colon (ODO3921) | 2.4 | 2.2 |
| 83238 CC NAT (ODO3921) | 2.0 | 1.9 |
| 83241 CC from Partial Hepatectomy | 0.0 | 0.0 |
| (ODO4309) | | |
| 83242 Liver NAT (ODO4309) | 0.2 | 0.3 |
| 87472 Colon mets to lung (OD04451-01) | 0.0 | 0.0 |
| 87473 Lung NAT (OD04451-02) | 0.9 | 0.6 |
| Normal Prostate Clontech A+ 6546-1 | 2.7 | 3.0 |
| 84140 Prostate Cancer (OD04410) | 1.5 | 1.4 |
| 84141 Prostate NAT (OD04410) | 6.5 | 8.0 |
| 87073 Prostate Cancer (OD04720-01) | 5.9 | 6.1 |
| 87074 Prostate NAT (OD04720-02) | 14.1 | 12.6 |
| Normal Lung GENPAK 061010 | 3.3 | 3.5 |
| 83239 Lung Met to Muscle (ODO4286) | 0.2 | 0.2 |
| 83240 Muscle NAT (ODO4286) | 0.0 | 0.0 |
| 84136 Lung Malignant Cancer (OD03126) | 7.9 | 6.0 |
| 84137 Lung NAT (OD03126) | 1.8 | 2.5 |
| 84871 Lung Cancer (OD04404) | 24.8 | 21.6 |
| 84872 Lung NAT (OD04404) | 1.8 | 1.7 |
| 84875 Lung Cancer (OD04565) | 0.7 | 1.2 |
| 84876 Lung NAT (OD04565) | 0.5 | 0.7 |
| 85950 Lung Cancer (OD04237-01) | 13.5 | 12.5 |
| 85970 Lung NAT (OD04237-02) | 1.4 | 1.0 |
| 83255 Ocular Mel Met to Liver (ODO4310) | 0.0 | 0.0 |
| 83256 Liver NAT (ODO4310) | 0.2 | 0.4 |
| 84139 Melanoma Mets to Lung (ODO4321) | 0.4 | 0.2 |
| 84138 Lung NAT (OD04321) | 2.5 | 1.2 |
| Normal Kidney GENPAK 061008 | 0.2 | 0.0 |
| 83786 Kidney Ca, Nuclear grade 2 | 0.0 | 0.0 |
| (OD04338) | | |
| 83787 Kidney NAT (OD04338) | 0.2 | 0.2 |
| 83788 Kidney Ca Nuclear grade ½ | 0.0 | 0.0 |
| (OD04339) | | |
| 83789 Kidney NAT (OD04339) | 0.0 | 0.0 |
| 83790 Kidney Ca, Clear cell type | 0.1 | 0.2 |
| (OD04340) | | |
| 83791 Kidney NAT (OD04340) | 0.0 | 0.2 |
| 83792 Kidney Ca, Nuclear grade 3 | 0.0 | 0.0 |
| (OD04348) | | |
| 83793 Kidney NAT (OD04348) | 0.0 | 0.1 |
| 87474 Kidney Cancer (OD04622-01) | 0.4 | 0.4 |
| 87475 Kidney NAT (OD04622-03) | 0.0 | 0.0 |
| 85973 Kidney Cancer (OD04450-01) | 0.0 | 0.0 |
| 85974 Kidney NAT (OD04450-03) | 0.0 | 0.0 |
| Kidney Cancer Clontech 8120607 | 0.4 | 0.3 |
| Kidney NAT Clontech 8120608 | 0.0 | 0.0 |
| Kidney Cancer Clontech 8120613 | 0.0 | 0.0 |
| Kidney NAT Clontech 8120614 | 0.0 | 0.0 |
| Kidney Cancer Clontech 9010320 | 0.0 | 0.0 |
| Kidney NAT Clontech 9010321 | 0.0 | 0.0 |
| Normal Uterus GENPAK 061018 | 0.1 | 0.0 |
| Uterus Cancer GENPAK 064011 | 0.5 | 0.6 |
| Normal Thyroid Clontech A+ 6570-1 | 0.5 | 0.4 |
| Thyroid Cancer GENPAK 064010 | 0.0 | 0.0 |
| Thyroid Cancer INVITROGEN A302152 | 0.0 | 0.0 |
| Thyroid NAT INVITROGEN A302153 | 0.2 | 0.1 |
| Normal Breast GENPAK 061019 | 5.5 | 5.6 |
| 84877 Breast Cancer (OD04566) | 0.5 | 0.7 |
| 85975 Breast Cancer (OD04590-01) | 3.0 | 3.9 |
| 85976 Breast Cancer Mets (OD04590-03) | 1.4 | 1.7 |
| 87070 Breast Cancer Metastasis | 100.0 | 100.0 |
| (OD04655-05) | | |
| GENPAK Breast Cancer 064006 | 1.7 | 2.0 |
| Breast Cancer Res. Gen. 1024 | 0.8 | 0.8 |
| Breast Cancer Clontech 9100266 | 3.5 | 4.1 |
| Breast NAT Clontech 9100265. | 3.5 | 4.2 |
| Breast Cancer INVITROGEN A209073 | 0.7 | 0.7 |
| Breast NAT INVITROGEN A2090734 | 1.1 | 1.3 |
| Normal Liver GENPAK 061009 | 2.0 | 1.7 |
| Liver Cancer GENPAK 064003 | 0.0 | 0.0 |
| Liver Cancer Research Genetics RNA 1025 | 0.3 | 0.3 |
| Liver Cancer Research Genetics RNA 1026 | 0.0 | 0.0 |
| Paired Liver Cancer Tissue Research | 0.2 | 0.1 |
| Genetics RNA 6004-T | | |
| Paired Liver Tissue Research | 0.0 | 0.1 |
| Genetics RNA 6004-N | | |
| Paired Liver Cancer Tissue Research | 0.0 | 0.0 |
| Genetics RNA 6005-T | | |
| Paired Liver Tissue Research Genetics | 0.1 | 0.1 |
| RNA 6005-N | | |
| Normal Bladder GENPAK 061001 | 0.2 | 0.1 |
| Bladder Cancer Research Genetics | 3.0 | 3.1 |
| RNA 1023 | | |
| Bladder Cancer INVITROGEN A302173 | 1.0 | 0.8 |
| 87071 Bladder Cancer (OD04718-01) | 0.0 | 0.0 |
| 87072 Bladder Normal Adjacent | 4.1 | 3.4 |
| (OD04718-03) | | |
| Normal Ovary Res. Gen. | 0.0 | 0.0 |
| Ovarian Cancer GENPAK 064008 | 1.6 | 1.4 |
| 87492 Ovary Cancer (OD04768-07) | 0.0 | 0.0 |
| 87493 Ovary NAT (OD04768-08) | 0.0 | 0.0 |
| Normal Stomach GENPAK 061017 | 1.0 | 1.7 |
| Gastric Cancer Clontech 9060358 | 0.2 | 0.2 |
| NAT Stomach Clontech 9060359 | 0.1 | 0.2 |
| Gastric Cancer Clontech 9060395 | 0.4 | 0.7 |
| NAT Stomach Clontech 9060394 | 0.4 | 0.4 |
| Gastric Cancer Clontech 9060397 | 0.1 | 0.3 |
| NAT Stomach Clontech 9060396 | 0.1 | 0.2 |
| Gastric Cancer GENPAK 064005 | 1.0 | 1.3 |

TABLE 17
Panel 3D

| | Relative | | Relative |
|---|---|---|---|
| | Expression (%) | | Expression (%) |
| | 3dtm5226f_{—} | | 3dtm5226f_{—} |
| Tissue Name | ag1094 | Tissue Name | ag1094 |
| 94905_Daoy_Medulloblastoma/ | 0.0 | 94954_Ca Ski_Cervical | 0.1 |
| Cerebellum_sscDNA | | epidermoid carcinoma | |
| | | (metastasis)_sscDNA | |
| 94906_TE671_Medulloblastom/ | 0.2 | 94955_ES-2_Ovarian clear cell | 0.0 |
| Cerebellum_sscDNA | | carcinoma_sscDNA | |
| 94907_D283 | 0.2 | 94957_Ramos/6h stim_{—} | 0.1 |
| Med_Medulloblastoma/Cerebellum_{—} | | Stimulated with | |
| sscDNA | | PMA/ionomycin 6h_sscDNA | |
| 94908_PFSK-1_Primitive | 1.6 | 94958_Ramos/14h stim_{—} | 0.0 |
| Neuroectodermal/Cerebellum_sscDNA | | Stimulated with | |
| | | PMA/ionomycin 14h_sscDNA | |
| 94909_XF-498_CNS_sscDNA | 30.4 | 94962_MEG-01_Chronic | 1.7 |
| | | myelogenous leukemia | |
| | | (megokaryoblast)_sscDNA | |
| 94910_SNB- | 0.7 | 94963_Raji_Burkitt's | 0.0 |
| 78_CNS/glioma_sscDNA | | lymphoma_sscDNA | |
| 94911_SF- | 0.0 | 94964_Daudi_Burkitt's | 0.0 |
| 268_CNS/glioblastoma_sscDNA | | lymphoma_sscDNA | |
| 94912_T98G_Glioblastoma_sscDNA | 3.3 | 94965_U266_B-cell | 0.2 |
| | | plasmacytoma/myeloma_sscDNA | |
| 96776_SK-N- | 22.4 | 94968_CA46_Burkitt's | 0.0 |
| SH_Neuroblastoma | | lymphoma_sscDNA | |
| (metastasis)_sscDNA | | | |
| 94913_SF- | 27.2 | 94970_RL_non-Hodgkin's B- | 0.1 |
| 295_CNS/glioblastoma_sscDNA | | cell lymphoma_sscDNA | |
| 94914_Cerebellum_sscDNA | 6.7 | 94972_JM1_pre-B-cell | 0.0 |
| | | lymphoma/leukemia_sscDNA | |
| 96777_Cerebellum_sscDNA | 0.0 | 94973_Jurkat_T cell | 0.0 |
| | | leukemia_sscDNA | |
| 94916_NCI- | 21.9 | 94974_TF- | 2.1 |
| H292_Mucoepidermoid lung | | 1_Erythroleukemia_sscDNA | |
| carcinoma_sscDNA | | | |
| 94917_DMS-114_Small cell | 2.4 | 94975_HUT 78_T-cell | 0.0 |
| lung cancer_sscDNA | | lymphoma_sscDNA | |
| 94918_DMS-79_Small cell | 0.0 | 94977_U937_Histiocytic | 0.0 |
| lung | | lymphoma_sscDNA | |
| cancer/neuroendocrine_sscDNA | | | |
| 94919_NCI-H146_Small cell | 100.0 | 94980_KU-812_Myelogenous | 8.2 |
| lung | | leukemia_sscDNA | |
| cancer/neuroendocrine_sscDNA | | | |
| 94920_NCI-H526_Small cell | 0.0 | 94981_769-P_Clear cell renal | 0.0 |
| lung | | carcinoma_sscDNA | |
| cancer/neuroendocrine_sscDNA | | | |
| 94921_NCI-N417_Small cell | 0.0 | 94983_Caki-2_Clear cell renal | 0.3 |
| lung | | carcinoma_sscDNA | |
| cancer/neuroendocrine_sscDNA | | | |
| 94923_NCI-H82_Small cell | 0.3 | 94984_SW 839_Clear cell renal | 0.0 |
| lung | | carcinoma_sscDNA | |
| cancer/neuroendocrine_sscDNA | | | |
| 94924_NCI-H157_Squamous | 0.0 | 94986_G401_Wilms' | 0.0 |
| cell lung cancer | | tumor_sscDNA | |
| (metastasis)_sscDNA | | | |
| 94925_NCI-H1155_Large cell | 65.1 | 94987_Hs766T_Pancreatic | 4.9 |
| lung | | carcinoma (LN | |
| cancer/neuroendocrine_sscDNA | | metastasis)_sscDNA | |
| 94926_NCI-H1299_Large cell | 0.0 | 94988_CAPAN-1_Pancreatic | 0.3 |
| lung | | adenocarcinoma (liver | |
| cancer/neuroendocrine_sscDNA | | metastasis)_sscDNA | |
| 94927_NCI-H727_Lung | 13.8 | 94989_SU86.86_Pancreatic | 1.5 |
| carcinoid_sscDNA | | carcinoma (liver | |
| | | metastasis)_sscDNA | |
| 94928_NCI-UMC-11_Lung | 28.7 | 94990_BxPC-3_Pancreatic | 23.7 |
| carcinoid_sscDNA | | adenocarcinoma_sscDNA | |
| 94929_LX-1_Small cell lung | 0.7 | 94991_HPAC_Pancreatic | 76.8 |
| cancer_sscDNA | | adenocarcinoma_sscDNA | |
| 94930_Colo-205_Colon | 0.0 | 94992_MIA PaCa-2_Pancreatic | 0.5 |
| cancer_sscDNA | | carcinoma_sscDNA | |
| 94931_KM12_Colon | 0.1 | 94993_CFPAC-1_Pancreatic | 0.5 |
| cancer_sscDNA | | ductal | |
| | | adenocarcinoma_sscDNA | |
| 94932_KM20L2_Colon | 7.3 | 94994_PANC-1_Pancreatic | 6.9 |
| cancer_sscDNA | | epithelioid ductal | |
| | | carcinoma_sscDNA | |
| 94933_NCI-H716_Colon | 80.1 | 94996_T24_Bladder carcinma | 4.1 |
| cancer_sscDNA | | (transitional cell)_sscDNA | |
| 94935_SW-48_Colon | 0.3 | 94997_5637_Bladder | 1.0 |
| adenocarcinoma_sscDNA | | carcinoma_sscDNA | |
| 94936_SW1116_Colon | 0.0 | 94998_HT-1197_Bladder | 2.4 |
| adenocarcinoma_sscDNA | | carcinoma_sscDNA | |
| 94937_LS 174T_Colon | 0.0 | 94999_UM-UC-3_Bladder | 0.0 |
| adenocarcinoma_sscDNA | | carcinma (transitional | |
| | | cell)_sscDNA | |
| 94938_SW-948_Colon | 0.6 | 95000_A204_Rhabdomyosarcoma_{—} | 0.3 |
| adenocarcinoma_sscDNA | | sscDNA | |
| 94939_SW-480_Colon | 0.0 | 95001_HT- | 0.2 |
| adenocarcinoma_sscDNA | | 1080_Fibrosarcoma_sscDNA | |
| 94940_NCI-SNU-5_Gastric | 0.0 | 95002_MG-63_Osteosarcoma | 0.2 |
| carcinoma_sscDNA | | (bone)_sscDNA | |
| 94941_KATO III_Gastric | 0.0 | 95003_SK-LMS- | 0.0 |
| carcinoma_sscDNA | | 1_Leiomyosarcoma | |
| | | (vulva)_sscDNA | |
| 94943_NCI-SNU-16_Gastric | 1.7 | 95004_SJRH30_Rhabdomyosarcoma | 0.2 |
| carcinoma_sscDNA | | (met to bone | |
| | | marrow)_sscDNA | |
| 94944_NCI-SNU-1_Gastric | 0.0 | 95005_A431_Epidermoid | 0.0 |
| carcinoma_sscDNA | | carcinoma_sscDNA | |
| 94946_RF-1_Gastric | 0.0 | 95007_WM266- | 0.4 |
| adenocarcinoma_sscDNA | | 4_Melanoma_sscDNA | |
| 94947_RF-48_Gastric | 0.0 | 95010_DU 145_Prostate | 0.0 |
| adenocarcinoma_sscDNA | | carcinoma (brain | |
| | | metastasis)_sscDNA | |
| 96778_MKN-45_Gastric | 0.1 | 95012_MDA-MB-468_Breast | 1.3 |
| carcinoma_sscDNA | | adenocarcinoma_sscDNA | |
| 94949_NCI-N87_Gastric | 2.4 | 95013_SCC-4_Squamous cell | 0.2 |
| carcinoma_sscDNA | | carcinoma of tongue_sscDNA | |
| 94951_OVCAR-5_Ovarian | 0.0 | 95014_SCC-9_Squamous cell | 0.0 |
| carcinoma_sscDNA | | carcinoma of tongue_sscDNA | |
| 94952_RL95-2_Uterine | 2.8 | 95015_SCC-15_Squamous cell | 0.3 |
| carcinoma_sscDNA | | carcinoma of tongue_sscDNA | |
| 94953_HelaS3_Cervical | 0.2 | 95017_CAL 27_Squamous cell | 1.6 |
| adenocarcinoma_sscDNA | | carcinoma of tongue_sscDNA | |

TABLE 18
Panel 4D

| | Relative | | Relative |
|---|---|---|---|
| | Expression | | Expression |
| | (%) | | (%) |
| | 4Dtm2495f_{—} | | 4Dtm2495f_{—} |
| Tissue Name | ag1094 | Tissue Name | ag1094 |
| 93768_Secondary Th1_anti- | 0.0 | 93100_HUVEC | 0.0 |
| CD28/anti-CD3 | | (Endothelial)_IL-1b | |
| 93769_Secondary Th2_anti- | 0.1 | 93779_HUVEC | 0.0 |
| CD28/anti-CD3 | | (Endothelial)_IFN gamma | |
| 93770_Secondary Tr1_anti- | 0.0 | 93102_HUVEC | 0.0 |
| CD28/anti-CD3 | | (Endothelial)_TNF alpha + IFN | |
| | | gamma | |
| 93573_Secondary Th1_resting | 0.0 | 93101_HUVEC | 0.3 |
| day 4-6 in IL-2 | | (Endothelial)_TNF alpha + IL4 | |
| 93572_Secondary Th2_resting | 0.0 | 93781_HUVEC | 0.0 |
| day 4-6 in IL-2 | | (Endothelial)_IL-11 | |
| 93571_Secondary Tr1_resting | 0.0 | 93583_Lung Microvascular | 0.0 |
| day 4-6 in IL-2 | | Endothelial Cells_none | |
| 93568_primary Th1_anti- | 0.0 | 93584_Lung Microvascular | 0.0 |
| CD28/anti-CD3 | | Endothelial Cells_TNFa (4 ng/ml) | |
| | | and IL1b (1 ng/ml) | |
| 93569_primary Th2_anti- | 0.0 | 92662_Microvascular Dermal | 0.0 |
| CD28/anti-CD3 | | endothelium_none | |
| 93570_primary Tr1_anti- | 0.0 | 92663_Microsvasular Dermal | 0.0 |
| CD28/anti-CD3 | | endothelium_TNFa (4 ng/ml) | |
| | | and IL1b (1 ng/ml) | |
| 93565_primary Th1_resting dy | 0.0 | 93773_Bronchial | 3.7 |
| 4-6 in IL-2 | | epithelium_TNFa (4 ng/ml) and | |
| | | IL1b (1 ng/ml)** | |
| 93566_primary Th2_resting dy | 0.0 | 93347_Small Airway | 4.5 |
| 4-6 in IL-2 | | Epithelium_none | |
| 93567_primary Tr1_resting dy | 0.0 | 93348_Small Airway | 1.7 |
| 4-6 in IL-2 | | Epithelium_TNFa (4 ng/ml) | |
| | | and IL1b (1 ng/ml) | |
| 93351_CD45RA CD4 | 0.0 | 92668_Coronery Artery | 0.0 |
| lymphocyte_anti-CD28/anti- | | SMC_resting | |
| CD3 | | | |
| 93352_CD45RO CD4 | 0.0 | 92669_Coronery Artery | 0.0 |
| lymphocyte_anti-CD28/anti- | | SMC_TNFa (4 ng/ml) and IL1b | |
| CD3 | | (1 ng/ml) | |
| 93251_CD8 Lymphocytes_anti- | 0.0 | 93107_astrocytes_resting | 8.5 |
| CD28/anti-CD3 | | | |
| 93353_chronic CD8 | 0.0 | 93108_astrocytes_TNFa (4 ng/ml) | 0.2 |
| Lymphocytes 2ry_resting dy 4-6 | | and IL1b (1 ng/ml) | |
| in IL-2 | | | |
| 93574_chronic CD8 | 0.3 | 92666_KU-812 | 8.3 |
| Lymphocytes 2ry_activated | | (Basophil)_resting | |
| CD3/CD28 | | | |
| 93354_CD4_none | 0.0 | 92667_KU-812 | 100.0 |
| | | (Basophil)_PMA/ionoycin | |
| 93252_Secondary | 0.0 | 93579_CCD1106 | 70.2 |
| Th1/Th2/Tr1 anti-CD95 CH11 | | (Keratinocytes)_none | |
| 93103_LAK cells_resting | 0.0 | 93580_CCD1106 | 3.0 |
| | | (Keratinocytes)_TNFa and | |
| | | IFNg** | |
| 93788_LAK cells_IL-2 | 0.0 | 93791_Liver Cirrhosis | 1.3 |
| 93787_LAK cells_IL-2 + IL-12 | 4.2 | 93792_Lupus Kidney | 0.0 |
| 93789_LAX cells_IL-2 + IFN | 0.0 | 93577_NCI-H292 | 16.6 |
| gamma | | | |
| 93790_LAK cells_IL-2 + IL-18 | 0.0 | 93358_NCI-H292_IL-4 | 10.4 |
| 93104_LAK | 0.0 | 93360_NCI-H292_IL-9 | 20.2 |
| cells_PMA/ionomycin and IL- | | | |
| 18 | | | |
| 93578_NK Cells IL-2_resting | 0.0 | 93359_NCI-H292_IL-13 | 6.3 |
| 93109_Mixed Lymphocyte | 0.0 | 93357_NCI-H292_IFN gamma | 8.5 |
| Reaction_Two Way MLR | | | |
| 93110_Mixed Lymphocyte | 0.0 | 93777_HPAEC_- | 0.0 |
| Reaction_Two Way MLR | | | |
| 93111_Mixed Lymphocyte | 0.0 | 93778_HPAEC_IL-1beta/TNA | 0.0 |
| Reaction_Two Way MLR | | alpha | |
| 93112_Mononuclear Cells | 0.0 | 93254_Normal Human Lung | 36.3 |
| (PBMCs)_resting | | Fibroblast_none | |
| 93113_Mononuclear Cells | 0.0 | 93253_Normal Human Lung | 18.6 |
| (PBMCs)_PWM | | Fibroblast_TNFa (4 ng/ml) and | |
| | | IL-1b (1 ng/ml) | |
| 93114_Mononuclear Cells | 0.0 | 93257_Normal Human Lung | 16.0 |
| (PBMCs)_PHA-L | | Fibroblast_IL-4 | |
| 93249_Ramos (B cell)_none | 0.0 | 93256_Normal Human Lung | 4.4 |
| | | Fibroblast_IL-9 | |
| 93250_Ramos (B | 0.3 | 93255_Normal Human Lung | 11.4 |
| cell)_ionomycin | | Fibroblast_IL-13 | |
| 93349_B lymphocytes_PWM | 0.0 | 93258_Normal Human Lung | 36.9 |
| | | Fibroblast_IFN gamma | |
| 93350_B lymphoytes_CD40L | 0.0 | 93106_Dermal Fibroblasts | 0.1 |
| and IL-4 | | CCD1070_resting | |
| 92665_EOL-1 | 0.0 | 93361_Dermal Fibroblasts | 0.0 |
| (Eosinophil)_dbcAMP | | CCD1070_TNF alpha 4 ng/ml | |
| differentiated | | | |
| 93248_EOL-1 | 0.0 | 93105_Dermal Fibroblasts | 0.0 |
| (Eosinophil)_dbcAMP/PMAion | | CCD1070_IL-1beta 1 ng/ml | |
| omycin | | | |
| 93356_Dendritic Cells_none | 0.0 | 93772_dermal fibroblast_IFN | 0.3 |
| | | gamma | |
| 93355_Dendritic Cells_LPS | 0.0 | 93771_dermal fibroblast_IL-4 | 0.0 |
| 100 ng/ml | | | |
| 93775_Dendritic Cells_anti- | 0.0 | 93260_IBD Colitis 2 | 0.3 |
| CD40 | | | |
| 93774_Monocytes_resting | 0.0 | 93261_IBD Crohns | 0.3 |
| 93776_Monocytes_LPS 50 ng/ml | 0.0 | 735010_Colon_normal | 11.4 |
| 93581_Macrophages_resting | 41.8 | 735019_Lung_none | 5.3 |
| 93582_Macrophages_LPS 100 ng/ml | 0.1 | 64028-1_Thymus_none | 1.2 |
| 93098_HUVEC | 1.7 | 64030-1_Kidney_none | 2.0 |
| (Endothelial)_none | | | |
| 93099_HUVEC | 0.0 | | |
| (Endothelial)_starved | | | |

Panel 1 Summary: Ag273b Expression of the NOV1a gene is highest in a metastatic prostate cancer cell line PC-3 (CT=26.8). There is also substantial expression of this gene in a number of lung cancer cell lines and brain cancer cell lines. Thus, expression of the NOV1a gene could be used to distinguish lung, prostate or brain cancer cell lines from other samples. In addition, therapeutic inhibition of this gene product, through the use of small molecule drugs or antibodies, might have benefit in the treatment of lung, prostate or brain cancer. Among other normal tissues this gene is also low to moderately expressed in heart, colon, small intestine, trachea, salivary gland, fetal liver, and mammary gland.

The NOV1a gene encodes a novel insulin-like growth factor binding protein acid labile subunit. Among CNS tissues, this gene is expressed at moderate levels in cerebellum and thalamus. Insulin-like growth factor (IGF) has been shown to have neuroprotective effects, as is currently under investigation as a biopharmaceutical for the treatment of amyotropic lateral sclerosis. In serum, IGF is bound to both IGF-binding protein (IGFBP) and the acid labile subunit (IGFBP-ALS). In the brain, glia produce IGFBP; however the IGFBP-ALS has not been detected in the CNS. Therefore, the NOV1a gene may represent the CNS equivalent of IGFBP-ALS. Because of the neuroprotective effects of IGF, therapeutic modulation of this gene or its protein product may be useful in treating diseases in which neuronal death/degeneration occur such as amyotropic lateral sclerosis, multiple sclerosis, Alzheimer's disease, Parkinson's disease, Huntington's disease, spinocerebellar ataxia, or CNS injury such as stroke, head or spinal cord trauma.

Panel 1.3D Summary: Ag1094 Results from two experiments using the same probe/primer set are in excellent agreement and are consistent with what is observed in Panel 1. Expression of the NOV1a gene is highest in a lung cancer cell line (CT=28). There is also substantial expression of this gene in a number of additional lung cancer cell lines and brain cancer cell lines as well as in a metastatic prostate cancer cell line. Thus, NOV1a gene expression could be used to distinguish prostate, lung or brain cancer cell lines from other samples. In addition, therapeutic inhibition of this gene product, through the use of small molecule drugs or antibodies, might have benefit in the treatment of prostate, lung or brain cancer.

Among CNS tissues, there is low but significant expression in thalamus and cerebellum. Please see Panel 1 summary for description of potential utility of this gene in the CNS.

Panel 2D Summary: Ag1094 Results from two experiments using the same probe/primer set are in good agreement. Expression of the NOV1a gene is highest in a metastatic breast cancer sample (CT=26-27). In addition, several other breast cancer and lung cancer samples show increased expression when compared to their normal adjacent margin samples. This observation is consistent with the results in Panel 1.3D that show higher NOV1a gene expression in lung cancer cell lines. Thus, expression of this gene might be used to distinguish breast cancer or lung cancer tissue from their normal counterparts and might be of diagnostic value. Moreover, therapeutic modulation of the NOV1a gene or its gene product, through the use of small molecule drugs or antibodies, may be of benefit for treatment of breast or lung cancer.

Panel 3D Summary: Ag1094 Expression of the NOV1a gene is highest in a small cell lung cancer cell line (CT=28.5). In addition, there is significant expression of this gene in other lung cancer cell lines as well as in several brain cancer and pancreatic cancer cell lines. These results are consistent with what is observed in the other panels. Thus, the expression of the NOV1a gene may be used to distinguish lung, breast or pancreas cancer cell line samples from other tissues. Moreover, therapeutic modulation of this gene or gene product, through the use of small molecule drugs or antibodies, may be of benefit for treatment of lung, breast or pancreatic cancer.

Panel 4D Summary: Ag1094 The NOV1a gene is expressed at high levels in basophils (CT 28.3) as well as in keratinocytes and normal lung fibroblasts (independently of their activation status). In addition, this gene is expressed at a lower level in a muco-epidermoid cell line (H292). Expression of the NOV1a gene is also found in normal lung which is consistent with the data from Panel 1.3D. The protein encoded by this transcript is a homolog of insulin-like growth factor binding protein acid labile subunit, a component of the systemic insulin-like growth factor-binding protein (IGFBP) complex. Therefore, this gene may play an important role in the biology of circulating IGFs. IGFs are involved in a wide array of cellular processes such as proliferation, prevention of apoptosis, and differentiation. Thus, the NOV1a gene may be a suitable target for protein therapeutic to modulate locally the mitogenic effect of IGF and could be useful in the treatment of emphysema, COPD, or skin related disease.

References:

1. Mewar R., McMorris F. A. (1997) Expression of insulin-like growth factor-binding protein messenger RNAs in developing rat oligodendrocytes and astrocytes. J. Neurosci. Res 50:721-728.

Insulin-like growth factors, IGF-I and IGF-II, are potent regulators of oligodendrocyte development. Most of the IGF present in vivo is bound to members of a family of six high-affinity IGF-binding proteins (IGFBPs), which can either potentiate or inhibit IGF action, depending on other conditions. Additionally, serum contains a structurally unrelated protein, acid-labile sub-unit (ALS), which forms a ternary complex with IGF and IGFBP3. In this study, reverse-transcriptase polymerase chain reaction (RT-PCR) was used to examine the expression of mRNAs for IGFBP 1-6 and ALS in purified populations of oligodendroglial cells and astrocytes. Astrocytes express all six IGFBPs. A2B5+/O4− oligodendrocyte precursors, O4+/O1− intermediate precursors, and O1+ oligodendrocytes express IGFBP3, 5, and 6, while IGFBP4 is expressed in oligodendrocyte precursors but not at more mature stages. They were unable to detect ALS mRNA in whole brain or in cultured oligodendroglial cells. The presence of differentially expressed IGFBPs in developing oligodendrocytes and astrocytes could significantly affect the biological activity of IGF-I and IGF-II in the central nervous system and the IGF-responsiveness of the IGFBP-expressing cells.

PMID: 9418960

2. Arnold P. M., Ma J. Y., Citron B. A., Zoubine M. N., Festoff B. W. (2000) Selective developmental regulation of gene expression for insulin-like growth factor-binding proteins in mouse spinal cord. Spine 25:1765-1770.

STUDY DESIGN: Prospective, randomized experimental study in mice. STUDY OBJECTIVE: To determine whether insulin-like growth factor binding proteins (IGFBPS) are present in mouse spinal cord and, if so, what role they play in its development. SUMMARY OF BACKGROUND DATA: Insulin-like growth factors are well recognized hormonal effectors of growth hormone and are expressed in the mammalian spinal cord. The IGFBPs are a group of six genetically distinct proteins that bind IGFs and modulate their bioactivity. They appear in the brain during development, localize to the neuromuscular junction, and promote motor neuron survival. The benefit of IGF-I in amyotrophic lateral sclerosis ALS and its potential use in preventing motor neuron apoptosis in spinal cord injury dictates that studies of the presence and response of IGFBPs in that tissue be performed. METHODS: The IGFBPs in mouse spinal cord were analyzed by Western ligand blot, Western immunoblot, and reverse transcription-polymerase chain reaction at various time points from embryonic day 14 to postnatal day 30. RESULTS: Three IGFBPs with molecular masses of 24, 28, and 32 kDa were found, the latter two being the most prominent. The data indicate that these are IGFBP-4, -5, and -2. CONCLUSION: Both IGFBP-2 and BP-5 are developmentally regulated in mouse spinal cord, with higher levels of those at early embryonic stages indicating their potential role in development of the mouse spinal cord.

PMID: 10888943

3. Corse A. M., Bilak M. M., Bilak S. R., Lehar M., Rothstein J. D., Kuncl R. W. (1999) Preclinical testing of neuroprotective neurotrophic factors in a model of chronic motor neuron degeneration. Neurobiol. Dis. 6:335-346.

Many neurotrophic factors have been shown to enhance survival of embryonic motor neurons or affect their response to injury. Few studies have investigated the potential effects of neurotrophic factors on more mature motor neurons that might be relevant for neurodegenerative diseases. Using organotypic spinal cord cultures from postnatal rats, researchers have demonstrated that insulin-like growth factor-I (IGF-I) and glial-derived neurotrophic factor (GDNF) significantly increase choline acetyltransferase (CHAT) activity, but brain-derived neurotrophic factor (BDNF), neurotrophin-4 (NT-4/5), and neurotrophin-3 (NT-3) do not. Surprisingly, ciliary neurotrophic factor (CNTF) actually reduces ChAT activity compared to age-matched control cultures. Neurotrophic factors have also been shown to alter the sensitivity of some neurons to glutamate neurotoxicity, a postulated mechanism of injury in the neurodegenerative disease, amyotrophic lateral sclerosis (ALS). Incubation of organotypic spinal cord cultures in the presence of the glutamate transport inhibitor threo-hydroxyaspartate (THA) reproducibly causes death of motor neurons which is glutamate-mediated. In this model of motor neuron degeneration, IGF-I, GDNF, and NT-4/5 are potently neuroprotective, but BDNF, CNTF, and NT-3 are not. The organotypic glutamate toxicity model appears to be the best preclinical predictor to date of success in human clinical trials in ALS.

NOV3a

Expression of gene NOV3a was assessed using the primer-probe set Ag2100, described in Table 19. Results from RTQ-PCR runs are shown in Tables 20, 21, 22, 23, and 24.

TABLE 19

Probe Name Ag2100

| | | | | | |
|---|---|---|---|---|---|
| | | | | Start | SEQ ID |
| Primers | Sequences | TM | Length | Position | NO: |

| | | | | | |
|---|---|---|---|---|---|
| Forward | 5′-AGATCCCTGGAACAGAGGATT-3′ | 59 | 21 | 2446 | 97 |
| Probe | TET-5′- | 67.9 | 26 | 2474 | 98 |
| | TGTCTGAAGCCAATAAACTTGCAGCA- | | | | |
| | 3′-TAMRA | | | | |
| Reverse | 5′-CCTTCATGTTCCTTTGGGTAA-3′ | 58.9 | 21 | 2513 | 99 |

TABLE 20
Panel 1.3D

| | Relative | | Relative |
|---|---|---|---|
| | Expression(%) | | Expression(%) |
| | 1.3dtm3300t_{—} | | 1.3dtm3300t_{—} |
| Tissue Name | ag2100 | Tissue Name | ag2100 |
| Liver adenocarcinoma | 11.7 | Kidney (fetal) | 1.8 |
| Pancreas | 0.0 | Renal ca. 786-0 | 7.1 |
| Pancreatic ca. CAPAN 2 | 3.2 | Renal ca. A498 | 3.7 |
| Adrenal gland | 1.4 | Renal ca. RXF 393 | 3.1 |
| Thyroid | 0.1 | Renal ca. ACHN | 4.4 |
| Salivary gland | 0.1 | Renal ca. UO-31 | 6.3 |
| Pituitary gland | 2.1 | Renal ca. TK-10 | 3.2 |
| Brain (fetal) | 2.1 | Liver | 0.0 |
| Brain (whole) | 24.7 | Liver (fetal) | 3.8 |
| Brain (amygdala) | 11.2 | Liver ca. (hepatoblast) HepG2 | 3.2 |
| Brain (cerebellum) | 2.7 | Lung | 0.3 |
| Brain (hippocampus) | 36.3 | Lung (fetal) | 0.9 |
| Brain (substantia nigra) | 1.5 | Lung ca. (small cell) LX-1 | 6.6 |
| Brain (thalamus) | 30.4 | Lung ca. (small cell) NCI-H69 | 8.5 |
| Cerebral Cortex | 100.0 | Lung ca. (s.cell var.) SHP-77 | 7.5 |
| Spinal cord | 2.5 | Lung ca. (large cell)NCI-H460 | 0.0 |
| CNS ca. (glio/astro) U87-MG | 6.4 | Lung ca. (non-sm. cell) A549 | 0.2 |
| CNS ca. (glio/astro) U-118-MG | 33.7 | Lung ca. (non-s.cell) NCI-H23 | 10.4 |
| CNS ca. (astro) SW1783 | 5.9 | Lung ca. (non-s.cell) HOP-62 | 1.4 |
| CNS ca.* (neuro; met) SK-N- | 14.5 | Lung ca. (non-s.cl) NCI-H522 | 5.3 |
| AS | | | |
| CNS ca. (astro) SF-539 | 7.4 | Lung ca. (squam.) SW 900 | 3.2 |
| CNS ca. (astro) SNB-75 | 5.8 | Lung ca. (squam.) NCI-H596 | 7.2 |
| CNS ca. (glio) SNB-19 | 1.0 | Mammary gland | 0.2 |
| CNS ca. (glio) U251 | 2.4 | Breast ca.* (pl. effusion) MCF-7 | 5.6 |
| CNS ca. (glio) SF-295 | 0.9 | Breast ca.* (pl.ef) MDA-MB- | 14.5 |
| | | 231 | |
| Heart (fetal) | 0.4 | Breast ca.* (pl. effusion) T47D | 2.4 |
| Heart | 0.1 | Breast ca. BT-549 | 6.8 |
| Fetal Skeletal | 3.4 | Breast ca. MDA-N | 14.0 |
| Skeletal muscle | 0.0 | Ovary | 2.2 |
| Bone marrow | 5.4 | Ovarian ca. OVCAR-3 | 2.5 |
| Thymus | 2.1 | Ovarian ca. OVCAR-4 | 0.8 |
| Spleen | 0.6 | Ovarian ca. OVCAR-5 | 2.7 |
| Lymph node | 0.4 | Ovarian ca. OVCAR-8 | 3.2 |
| Colorectal | 1.8 | Ovarian ca. IGROV-1 | 2.0 |
| Stomach | 1.0 | Ovarian ca.* (ascites) SK-OV-3 | 7.4 |
| Small intestine | 1.6 | Uterus | 0.0 |
| Colon ca. SW480 | 13.1 | Placenta | 0.2 |
| Colon ca.* (SW480 met)SW620 | 4.5 | Prostate | 0.2 |
| Colon ca. HT29 | 4.1 | Prostate ca.* (bone met)PC-3 | 2.0 |
| Colon ca. HCT-116 | 5.0 | Testis | 4.0 |
| Colon ca. CaCo-2 | 5.9 | Melanoma Hs688(A).T | 0.7 |
| 83219 CC Well to Mod Diff | 2.8 | Melanoma* (met) Hs688(B).T | 0.3 |
| (ODO3866) | | | |
| Colon ca. HCC-2998 | 3.7 | Melanoma UACC-62 | 0.5 |
| Gastric ca.* (liver met) NCI- | 2.3 | Melanoma M14 | 7.2 |
| N87 | | | |
| Bladder | 0.9 | Melanoma LOX IMVI | 2.8 |
| Trachea | 0.7 | Melanoma* (met) SK-MEL-5 | 5.8 |
| Kidney | 0.7 | Adipose | 0.2 |

TABLE 21
Panel 2.2

| | Relative | | Relative |
|---|---|---|---|
| | Expression(%) | | Expression(%) |
| | 2.2x4tm6379t_{—} | | 2.2x4tm6379t_{—} |
| Tissue Name | ag2100_b2 | Tissue Name | ag2100_b2 |
| Normal Colon GENPAK | 6.2 | 83793 Kidney NAT (OD04348) | 30.3 |
| 061003 | | | |
| 97759 Colon cancer (OD06064) | 13.4 | 98938 Kidney malignant cancer | 3.6 |
| | | (OD06204B) | |
| 97760 Colon cancer NAT | 9.0 | 98939 Kidney normal adjacent | 10.5 |
| (OD06064) | | tissue (OD06204E) | |
| 97778 Colon cancer (OD06159) | 4.5 | 85973 Kidney Cancer | 2.4 |
| | | (OD04450-01) | |
| 97779 Colon cancer NAT | 5.9 | 85974 Kidney NAT (OD04450- | 13.3 |
| (OD06159) | | 03) | |
| 98861 Colon cancer (OD06297- | 3.8 | Kidney Cancer Clontech | 6.6 |
| 04) | | 8120613 | |
| 98862 Colon cancer NAT | 10.0 | Kidney NAT Clontech 8120614 | 1.2 |
| (OD06297-015) | | | |
| 83237 CC Gr.2 ascend colon | 4.3 | Kidney Cancer Clontech | 1.6 |
| (ODO3921) | | 9010320 | |
| 83238 CC NAT (ODO3921) | 2.8 | Kidney NAT Clontech 9010321 | 4.5 |
| 97766 Colon cancer metastasis | 1.7 | Kidney Cancer Clontech | 0.5 |
| (OD06104) | | 8120607 | |
| 97767 Lung NAT (OD06104) | 3.1 | Kidney NAT Clontech 8120608 | 1.7 |
| 87472 Colon mets to lung | 9.6 | Normal Uterus GENPAK | 1.1 |
| (OD04451-01) | | 061018 | |
| 87473 Lung NAT (OD04451- | 3.2 | Uterus Cancer GENPAK | 1.5 |
| 02) | | 064011 | |
| Normal Prostate Clontech A+ | 1.2 | Normal Thyroid Clontech A+ | 0.0 |
| 6546-1 (8090438) | | 6570-1 (7080817) | |
| 84140 Prostate Cancer | 0.0 | Thyroid Cancer GENPAK | 0.6 |
| (OD04410) | | 064010 | |
| 84141 Prostate NAT | 0.7 | Thyroid Cancer INVITROGEN | 5.3 |
| (OD04410) | | A302152 | |
| Normal Ovary Res. Gen. | 2.8 | Thyroid NAT INVITROGEN | 0.0 |
| | | A302153 | |
| 98863 Ovarian cancer | 11.7 | Normal Breast GENPAK | 3.0 |
| (OD06283-03) | | 061019 | |
| 98865 Ovarian cancer | 3.0 | 84877 Breast Cancer | 8.1 |
| NAT/fallopian tube (OD06283- | | (OD04566) | |
| 07) | | | |
| Ovarian Cancer GENPAK | 1.1 | Breast Cancer Res. Gen. 1024 | 2.9 |
| 064008 | | | |
| 97773 Ovarian cancer | 0.9 | 85975 Breast Cancer | 14.7 |
| (OD06145) | | (OD04590-01) | |
| 97775 Ovarian cancer NAT | 0.0 | 85976 Breast Cancer Mets | 3.2 |
| (OD06145) | | (OD04590-03) | |
| 98853 Ovarian cancer | 15.8 | 87070 Breast Cancer Metastasis | 5.4 |
| (OD06455-03) | | (OD04655-05) | |
| 98854 Ovarian NAT | 1.8 | GENPAK Breast Cancer | 3.1 |
| (OD06455-07) Fallopian tube | | 064006 | |
| Normal Lung GENPAK 061010 | 1.2 | Breast Cancer Clontech | 2.6 |
| | | 9100266 | |
| 92337 Invasive poor diff. lung | 8.4 | Breast NAT Clontech 9100265 | 2.3 |
| adeno (ODO4945-01 | | | |
| 92338 Lung NAT (ODO4945- | 1.2 | Breast Cancer INVITROGEN | 1.8 |
| 03) | | A209073 | |
| 84136 Lung Malignant Cancer | 5.0 | Breast NAT INVITROGEN | 2.5 |
| (OD03126) | | A2090734 | |
| 84137 Lung NAT (OD03126) | 0.6 | 97763 Breast cancer | 17.1 |
| | | (OD06083) | |
| 90372 Lung Cancer | 10.1 | 97764 Breast cancer node | 14.6 |
| (OD05014A) | | metastasis (OD06083) | |
| 90373 Lung NAT (OD05014B) | 9.0 | Normal Liver GENPAK | 0.4 |
| | | 061009 | |
| 97761 Lung cancer (OD06081) | 10.1 | Liver Cancer Research Genetics | 0.0 |
| | | RNA 1026 | |
| 97762 Lung cancer NAT | 4.0 | Liver Cancer Research Genetics | 1.8 |
| (OD06081) | | RNA 1025 | |
| 85950 Lung Cancer (OD04237- | 4.1 | Paired Liver Cancer Tissue | 1.1 |
| 01) | | Research Genetics RNA 6004-T | |
| 85970 Lung NAT (OD04237- | 2.0 | Paired Liver Tissue Research | 2.5 |
| 02) | | Genetics RNA 6004-N | |
| 83255 Ocular Mel Met to Liver | 0.9 | Paired Liver Cancer Tissue | 1.6 |
| (ODO4310) | | Research Genetics RNA 6005-T | |
| 83256 Liver NAT (ODO4310) | 0.4 | Paired Liver Tissue Research | 0.0 |
| | | Genetics RNA 6005-N | |
| 84139 Melanoma Mets to Lung | 10.4 | Liver Cancer GENPAK 064003 | 0.7 |
| (OD04321) | | | |
| 84138 Lung NAT (OD04321) | 2.0 | Normal Bladder GENPAK | 2.9 |
| | | 061001 | |
| Normal Kidney GENPAK | 5.0 | Bladder Cancer Research | 1.5 |
| 061008 | | Genetics RNA 1023 | |
| 83786 Kidney Ca, Nuclear | 15.3 | Bladder Cancer INVITROGEN | 17.8 |
| grade 2 (OD04338) | | A302173 | |
| 83787 Kidney NAT (OD04338) | 5.1 | Normal Stomach GENPAK | 10.4 |
| | | 061017 | |
| 83788 Kidney Ca Nuclear grade | 100.0 | Gastric Cancer Clontech | 1.1 |
| ½ (OD04339) | | 9060397 | |
| 83789 Kidney NAT (OD04339) | 9.3 | NAT Stomach Clontech | 0.7 |
| | | 9060396 | |
| 83790 Kidney Ca, Clear cell | 14.0 | Gastric Cancer Clontech | 2.8 |
| type (OD04340) | | 9060395 | |
| 83791 Kidney NAT (OD04340) | 11.2 | NAT Stomach Clontech | 2.8 |
| | | 9060394 | |
| 83792 Kidney Ca, Nuclear | 9.0 | Gastric Cancer GENPAK | 6.0 |
| grade 3 (OD04348) | | 064005 | |

TABLE 22
Panel 3D

| | Relative | | Relative |
|---|---|---|---|
| | Expression(%) | | Expression(%) |
| | 3dx4tm5110t_{—} | | 3dx4tm5110t_{—} |
| Tissue Name | ag2100_a2 | Tissue Name | ag2100_a2 |
| 94905_Daoy_Medulloblastoma/ | 7.3 | 94954_Ca Ski_Cervical | 21.0 |
| Cerebellum_sscDNA | | epidermoid carcinoma | |
| | | (metastasis)_sscDNA | |
| 94906_TE671_Medulloblastom/ | 3.8 | 94955_ES-2_Ovarian clear cell | 11.7 |
| Cerebellum_sscDNA | | carcinoma_sscDNA | |
| 94907_D283 | 15.7 | 94957_Ramos/6h stim_{—} | 10.8 |
| Med_Medulloblastoma/Cerebellum_{—} | | Stimulated with | |
| sscDNA | | PMA/ionomycin 6h_sscDNA | |
| 94908_PFSK-1_Primitive | 11.2 | 94958_Ramos/14h stim_{—} | 6.2 |
| Neuroectodermal/Cerebellum_sscDNA | | Stimulated with | |
| | | PMA/ionomycin_14h_sscDNA | |
| 94909_XF-498_CNS_sscDNA | 21.2 | 94962_MEG-01_Chronic | 5.8 |
| | | myelogenous leukemia | |
| | | (megokaryoblast)_sscDNA | |
| 94910_SNB- | 11.3 | 94963_Raji_Burkitt's | 6.8 |
| 78_CNS/glioma_sscDNA | | lymphoma_sscDNA | |
| 94911_SF- | 7.6 | 94964_Daudi_Burkitt's | 14.7 |
| 268_CNS/glioblastoma_sscDNA | | lymphoma_sscDNA | |
| 94912_T98G_Glioblastoma_sscDNA | 12.0 | 94965_U266_B-cell | 5.1 |
| | | plasmacytoma/myeloma_sscDNA | |
| 96776_SK-N- | 5.6 | 94968_CA46_Burkitt's | 5.0 |
| SH_Neuroblastoma | | lymphoma_sscDNA | |
| (metastasis)_sscDNA | | | |
| 94913_SF- | 12.4 | 94970_RL_non-Hodgkin's B- | 3.8 |
| 295_CNS/glioblastoma_sscDNA | | cell lymphoma_sscDNA | |
| 94914_Cerebellum_sscDNA | 16.1 | 94972_JM1_pre-B-cell | 11.5 |
| | | lymphoma/leukemia_sscDNA | |
| 96777_Cerebellum_sscDNA | 3.6 | 94973_Jurkat_T cell | 12.5 |
| | | leukemia_sscDNA | |
| 94916_NCI- | 14.0 | 94974_TF- | 9.9 |
| H292_Mucoepidermoid lung | | 1_Erythroleukemia_sscDNA | |
| carcinoma_sscDNA | | | |
| 94917_DMS-114_Small cell | 10.3 | 94975_HUT 78_T-cell | 14.7 |
| lung cancer_sscDNA | | lymphoma_sscDNA | |
| 94918_DMS-79_Small cell | 100.0 | 94977_U937_Histiocytic | 8.1 |
| lung | | lymphoma_sscDNA | |
| cancer/neuroendocrine_sscDNA | | | |
| 94919_NCI-H146_Small cell | 14.2 | 94980_KU-812_Myelogenous | 17.7 |
| lung | | leukemia_sscDNA | |
| cancer/neuroendocrine_sscDNA | | | |
| 94920_NCI-H526_Small cell | 19.8 | 94981_769-P_Clear cell renal | 6.3 |
| lung | | carcinoma_sscDNA | |
| cancer/neuroendocrine_sscDNA | | | |
| 94921_NCI-N417_Small cell | 5.7 | 94983_Caki-2_Clear cell renal | 9.5 |
| lung | | carcinoma_sscDNA | |
| cancer/neuroendocrine_sscDNA | | | |
| 94923_NCI-H82_Small cell | 10.1 | 94984_SW 839_Clear cell renal | 5.2 |
| lung | | carcinoma_sscDNA | |
| cancer/neuroendocrine_sscDNA | | | |
| 94924_NCI-H157_Squamous | 13.8 | 94986_G401_Wilms' | 6.3 |
| cell lung cancer | | tumor_sscDNA | |
| (metastasis)_sscDNA | | | |
| 94925_NCI-H1155_Large cell | 36.0 | 94987_Hs766T_Pancreatic | 15.7 |
| lung | | carcinoma (LN | |
| cancer/neuroendocrine_sscDNA | | metastasis)_sscDNA | |
| 94926_NCI-H1299_Large cell | 22.7 | 94988_CAPAN-1_Pancreatic | 8.6 |
| lung | | adenocarcinoma (liver | |
| cancer/neuroendocrine_sscDNA | | metastasis)_sscDNA | |
| 94927_NCI-H727_Lung | 14.3 | 94989_SU86.86_Pancreatic | 14.0 |
| carcinoid_sscDNA | | carcinoma (liver | |
| | | metastasis)_sscDNA | |
| 94928_NCI-UMC-11_Lung | 25.8 | 94990_BxPC-3_Pancreatic | 9.4 |
| carcinoid_sscDNA | | adenocarcinoma_sscDNA | |
| 94929_LX-1_Small cell lung | 11.0 | 94991_HPAC_Pancreatic | 14.4 |
| cancer_sscDNA | | adenocarcinoma_sscDNA | |
| 94930_Colo-205_Colon | 12.7 | 94992_MIA PaCa-2_Pancreatic | 2.6 |
| cancer_sscDNA | | carcinoma_sscDNA | |
| 94931_KM12_Colon | 17.1 | 94993_CFPAC-1_Pancreatic | 38.5 |
| cancer_sscDNA | | ductal | |
| | | adenocarcinoma_sscDNA | |
| 94932_KM20L2_Colon | 7.0 | 94994_PANC-1_Pancreatic | 19.5 |
| cancer_sscDNA | | epithelioid ductal | |
| | | carcinoma_sscDNA | |
| 94933_NCI-H716_Colon | 19.4 | 94996_T24_Bladder carcinma | 9.0 |
| cancer_sscDNA | | (transitional cell)_sscDNA | |
| 94935_SW-48_Colon | 10.6 | 94997_5637_Bladder | 10.5 |
| adenocarcinoma_sscDNA | | carcinoma_sscDNA | |
| 94936_SW1116_Colon | 7.7 | 94998_HT-1197_Bladder | 4.8 |
| adenocarcinoma_sscDNA | | carcinoma_sscDNA | |
| 94937_LS 174T_Colon | 9.8 | 94999_UM-UC-3_Bladder | 13.3 |
| adenocarcinoma_sscDNA | | carcinma (transitional | |
| | | cell)_sscDNA | |
| 94938_SW-948_Colon | 1.4 | 95000_A204_Rhabdomyosarcoma_{—} | 15.2 |
| adenocarcinoma_sscDNA | | sscDNA | |
| 94939_SW-480_Colon | 7.6 | 95001_HT- | 11.9 |
| adenocarcinoma_sscDNA | | 1080_Fibrosarcoma_sscDNA | |
| 94940_NCI-SNU-5_Gastric | 14.8 | 95002_MG-63_Osteosarcoma | 7.3 |
| carcinoma_sscDNA | | (bone)_sscDNA | |
| 94941_KATO III_Gastric | 18.8 | 95003_SK-LMS- | 47.8 |
| carcinoma_sscDNA | | 1_Leiomyosarcoma | |
| | | (vulva)_sscDNA | |
| 94943_NCI-SNU-16_Gastric | 12.5 | 95004_SJRH30_Rhabdomyosarcoma | 10.2 |
| carcinoma_sscDNA | | (met to bone | |
| | | marrow)_sscDNA | |
| 94944_NCI-SNU-1_Gastric | 12.3 | 95005_A431_Epidermoid | 12.1 |
| carcinoma_sscDNA | | carcinoma_sscDNA | |
| 94946_RF-1_Gastric | 5.3 | 95007_WM266- | 21.8 |
| adenocarcinoma_sscDNA | | 4_Melanoma_sscDNA | |
| 94947_RF-48_Gastric | 7.7 | 95010_DU 145_Prostate | 0.2 |
| adenocarcinoma_sscDNA | | carcinoma (brain | |
| | | metastasis)_sscDNA | |
| 96778_MKN-45_Gastric | 11.7 | 95012_MDA-MB-468_Breast | 5.6 |
| carcinoma_sscDNA | | adenocarcinoma_sscDNA | |
| 94949_NCI-N87_Gastric | 9.3 | 95013_SCC-4_Squamous cell | 0.3 |
| carcinoma_sscDNA | | carcinoma of tongue_sscDNA | |
| 94951_OVCAR-5_Ovarian | 3.0 | 95014_SCC-9 Squamous cell | 0.3 |
| carcinoma_sscDNA | | carcinoma of tongue_sscDNA | |
| 94952_RL95-2_Uterine | 4.5 | 95015_SCC-15_Squamous cell | 0.2 |
| carcinoma_sscDNA | | carcinoma of tongue_sscDNA | |
| 94953_HelaS3_Cervical | 9.0 | 95017_CAL 27_Squamous cell | 19.8 |
| adenocarcinoma_sscDNA | | carcinoma of tongue_sscDNA | |

TABLE 23
Panel 4D

| | Relative | | Relative |
|---|---|---|---|
| | Expression (%) | | Expression (%) |
| | 4dtm3359t_{—} | | 4dtm3359t_{—} |
| Tissue Name | ag2100 | Tissue Name | ag2100 |
| 93768_Secondary Th1_anti- | 15.4 | 93100_HUVEC | 12.2 |
| CD28/anti-CD3 | | (Endothelial)_IL-1b | |
| 93769_Secondary Th2_anti- | 11.9 | 93779_HUVEC | 16.6 |
| CD28/anti-CD3 | | (Endothelial)_IFN gamma | |
| 93770_Secondary Tr1_anti- | 15.6 | 93102_HUVEC | 11.8 |
| CD28/anti-CD3 | | (Endothelial)_TNF alpha + IFN | |
| | | gamma | |
| 93573_Secondary Th1_resting | 4.9 | 93101_HUVEC | 11.4 |
| day 4-6 in IL-2 | | (Endothelial)_TNF alpha + IL4 | |
| 93572_Secondary Th2_resting | 3.3 | 93781_HUVEC | 8.2 |
| day 4-6 in IL-2 | | (Endothelial)_IL-11 | |
| 93571_Secondary Tr1_resting | 6.0 | 93583_Lung Microvascular | 7.3 |
| day 4-6 in IL-2 | | Endothelial Cells_none | |
| 93568_primary Th1_anti- | 13.6 | 93584_Lung Microvascular | 6.2 |
| CD28/anti-CD3 | | Endothelial Cells_TNFa (4 ng/ml) | |
| | | and IL1b (1 ng/ml) | |
| 93569_primary Th2_anti- | 12.0 | 92662_Microvascular Dermal | 23.3 |
| CD28/anti-CD3 | | endothelium_none | |
| 93570_primary Tr1_anti- | 22.2 | 92663_Microsvasular Dermal | 10.5 |
| CD28/anti-CD3 | | endothelium_TNFa (4 ng/ml) | |
| | | and IL1b (1 ng/ml) | |
| 93565_primary Th1_resting dy | 100.0 | 93773_Bronchial | 0.6 |
| 4-6 in IL-2 | | epithelium_TNFa (4 ng/ml) and | |
| | | IL1b (1 ng/ml)** | |
| 93566_primary Th2_resting dy | 37.9 | 93347_Small Airway | 1.6 |
| 4-6 in IL-2 | | Epithelium_none | |
| 93567_primary Tr1_resting dy | 29.3 | 93348_Small Airway | 7.4 |
| 4-6 in IL-2 | | Epithelium_TNFa (4 ng/ml) | |
| | | and IL1b (1 ng/ml) | |
| 93351_CD45RA CD4 | 13.6 | 92668_Coronery Artery | 4.4 |
| lymphocyte_anti-CD28/anti- | | SMC_resting | |
| CD3 | | | |
| 93352_CD45RO CD4 | 15.4 | 92669_Coronery Artery | 2.0 |
| lymphocyte_anti-CD28/anti- | | SMC_TNFa (4 ng/ml) and IL1b | |
| CD3 | | (1 ng/ml) | |
| 93251_CD8 Lymphocytes_anti- | 10.6 | 93107_astrocytes_resting | 1.3 |
| CD28/anti-CD3 | | | |
| 93353_chronic CD8 | 7.9 | 93108_astrocytes_TNFa (4 ng/ml) | 0.5 |
| Lymphocytes 2ry_resting dy 4-6 | | and IL1b (1 ng/ml) | |
| in IL-2 | | | |
| 93574_chronic CD8 | 17.3 | 92666_KU-812 | 22.4 |
| Lymphocytes 2ry_activated | | (Basophil)_resting | |
| CD3/CD28 | | | |
| 93354_CD4_none | 0.5 | 92667_KU-812 | 28.5 |
| | | (Basophil)_PMA/ionoycin | |
| 93252_Secondary | 17.1 | 93579_CCD1106 | 14.3 |
| Th1/Th2/Tr1_anti-CD95 CH11 | | (Keratinocytes)_none | |
| 93103_LAK cells_resting | 3.6 | 93580_CCD1106 | 18.4 |
| | | (Keratinocytes)_TNFa and | |
| | | IFNg** | |
| 93788_LAK cells_IL-2 | 16.8 | 93791_Liver Cirrhosis | 0.5 |
| 93787_LAK cells_IL-2 + IL-12 | 8.4 | 93792_Lupus Kidney | 3.3 |
| 93789_LAK cells_IL-2 + IFN | 16.4 | 93577_NCI-H292 | 29.5 |
| gamma | | | |
| 93790_LAK cells_IL-2 + IL-18 | 16.8 | 93358_NCI-H292_IL-4 | 27.7 |
| 93104_LAK | 0.6 | 93360_NCI-H292_IL-9 | 32.3 |
| cells_PMA/ionomycin and IL- | | | |
| 18 | | | |
| 93578_NK Cells IL-2_resting | 15.3 | 93359_NCI-H292_IL-13 | 13.4 |
| 93109_Mixed Lymphocyte | 1.8 | 93357_NCI-H292_IFN gamma | 11.0 |
| Reaction_Two Way MLR | | | |
| 93110_Mixed Lymphocyte | 6.1 | 93777_HPAEC_- | 8.5 |
| Reaction_Two Way MLR | | | |
| 93111_Mixed Lymphocyte | 10.1 | 93778_HPAEC_IL-1beta/TNA | 7.7 |
| Reaction_Two Way MLR | | alpha | |
| 93112_Mononuclear Cells | 0.1 | 93254_Normal Human Lung | 6.3 |
| (PBMCs)_resting | | Fibroblast_none | |
| 93113_Mononuclear Cells | 25.5 | 93253_Normal Human Lung | 9.0 |
| (PBMCs)_PWM | | Fibroblast_TNFa (4 ng/ml) and | |
| | | IL-1b (1 ng/ml) | |
| 93114_Mononuclear Cells | 24.0 | 93257_Normal Human Lung | 3.7 |
| (PBMCs)_PHA-L | | Fibroblast_IL-4 | |
| 93249_Ramos (B cell)_none | 17.7 | 93256_Normal Human Lung | 5.0 |
| | | Fibroblast_IL-9 | |
| 93250_Ramos (B | 92.0 | 93255_Normal Human Lung | 1.7 |
| cell)_ionomycin | | Fibroblast_IL-13 | |
| 93349_B lymphocytes_PWM | 48.6 | 93258_Normal Human Lung | 3.4 |
| | | Fibroblast_IFN gamma | |
| 93350_B lymphoytes_CD40L | 16.4 | 93106_Dermal Fibroblasts | 57.4 |
| and IL-4 | | CCD1070_resting | |
| 92665_EOL-1 | 10.5 | 93361_Dermal Fibroblasts | 79.0 |
| (Eosinophil)_dbcAMP | | CCD1070_TNF alpha 4 ng/ml | |
| differentiated | | | |
| 93248_EOL-1 | 7.0 | 93105_Dermal Fibroblasts | 21.8 |
| (Eosinophil)_dbcAMP/PMAionomycin | | CCD1070_IL-1beta 1 ng/ml | |
| 93356_Dendritic Cells_none | 0.5 | 93772_dermal fibroblast_IFN | 22.2 |
| | | gamma | |
| 93355_Dendritic Cells_LPS | 0.0 | 93771_dermal fibroblast_IL-4 | 45.7 |
| 100 ng/ml | | | |
| 93775_Dendritic Cells_anti- | 0.0 | 93260_IBD Colitis 2 | 0.9 |
| CD40 | | | |
| 93774_Monocytes resting | 0.2 | 93261_IBD Crohns | 1.0 |
| 93776_Monocytes_LPS 50 ng/ml | 0.0 | 735010_Colon_normal | 3.7 |
| 93581_Macrophages_resting | 4.4 | 735019_Lung_none | 1.5 |
| 93582_Macrophages_LPS 100 ng/ml | 0.6 | 64028-1_Thymus_none | 13.0 |
| 93098_HUVEC | 24.7 | 64030-1_Kidney_none | 31.2 |
| (Endothelial)_none | | | |
| 93099_HUVEC | 43.5 | | |
| (Endothelial)_starved | | | |

TABLE 24
AI_comprehensive_panel_v1.0

| | Relative Expression (%) | |
|---|---|---|
| | tm7130t_{—} | tm7159t_{—} |
| Tissue Name | ag2100_a2 | ag2100_b1 |
| 110967 COPD-F | 0.5 | 0.8 |
| 110980 COPD-F | 1.5 | 1.2 |
| 110968 COPD-M | 0.4 | 0.6 |
| 110977 COPD-M | 1.5 | 1.9 |
| 110989 Emphysema-F | 4.2 | 6.0 |
| 110992 Emphysema-F | 2.8 | 2.9 |
| 110993 Emphysema-F | 0.9 | 0.8 |
| 110994 Emphysema-F | 0.7 | 0.4 |
| 110995 Emphysema-F | 2.0 | 5.4 |
| 110996 Emphysema-F | 2.2 | 2.4 |
| 110997 Asthma-M | 1.9 | 3.1 |
| 111001 Asthma-F | 1.4 | 2.7 |
| 111002 Asthma-F | 1.0 | 1.0 |
| 111003 Atopic Asthma-F | 4.0 | 2.2 |
| 111004 Atopic Asthma-F | 16.6 | 17.0 |
| 111005 Atopic Asthma-F | 7.2 | 5.5 |
| 111006 Atopic Asthma-F | 0.9 | 0.7 |
| 111417 Allergy-M | 1.9 | 2.4 |
| 112347 Allergy-M | 0.0 | 0.0 |
| 112349 Normal Lung-F | 0.0 | 0.0 |
| 112357 Normal Lung-F | 6.1 | 6.0 |
| 112354 Normal Lung-M | 1.5 | 2.3 |
| 112374 Crohns-F | 2.9 | 5.2 |
| 112389 Match Control Crohns-F | 9.0 | 6.8 |
| 112375 Crohns-F | 2.5 | 3.8 |
| 112732 Match Control Crohns-F | 3.8 | 5.4 |
| 112725 Crohns-M | 0.1 | 0.7 |
| 112387 Match Control Crohns-M | 1.0 | 1.4 |
| 112378 Crohns-M | 0.0 | 0.0 |
| 112390 Match Control Crohns-M | 2.5 | 1.8 |
| 112726 Crohns-M | 3.8 | 5.9 |
| 112731 Match Control Crohns-M | 3.6 | 6.7 |
| 112380 Ulcer Col-F | 4.9 | 4.9 |
| 112734 Match Control Ulcer Col-F | 12.6 | 12.0 |
| 112384 Ulcer Col-F | 6.6 | 10.2 |
| 112737 Match Control Ulcer Col-F | 4.1 | 6.1 |
| 112386 Ulcer Col-F | 0.5 | 1.2 |
| 112738 Match Control Ulcer Col-F | 7.5 | 7.9 |
| 112381 Ulcer Col-M | 0.1 | 0.0 |
| 112735 Match Control Ulcer Col-M | 2.9 | 2.3 |
| 112382 Ulcer Col-M | 6.8 | 8.4 |
| 112394 Match Control Ulcer Col-M | 0.5 | 0.5 |
| 112383 Ulcer Col-M | 12.1 | 14.6 |
| 112736 Match Control Ulcer Col-M | 3.5 | 5.3 |
| 112423 Psoriasis-F | 1.4 | 1.1 |
| 112427 Match Control Psoriasis-F | 2.9 | 1.8 |
| 112418 Psoriasis-M | 0.8 | 0.8 |
| 112723 Match Control Psoriasis-M | 6.1 | 7.4 |
| 112419 Psoriasis-M | 1.0 | 1.3 |
| 112424 Match Control Psoriasis-M | 0.4 | 1.2 |
| 112420 Psoriasis-M | 1.8 | 2.4 |
| 112425 Match Control Psoriasis-M | 2.2 | 2.7 |
| 104689 (MF) OA Bone-Backus | 12.1 | 13.2 |
| 104690 (MF) Adj “Normal” Bone-Backus | 5.4 | 4.2 |
| 104691 (MF) OA Synovium-Backus | 43.3 | 35.7 |
| 104692 (BA) OA Cartilage-Backus | 0.9 | 0.4 |
| 104694 (BA) OA Bone-Backus | 16.8 | 16.7 |
| 104695 (BA) Adj “Normal” Bone-Backus | 6.5 | 6.1 |
| 104696 (BA) OA Synovium-Backus | 24.0 | 24.2 |
| 104700 (SS) OA Bone-Backus | 12.3 | 35.1 |
| 104701 (SS) Adj “Normal” Bone-Backus | 7.9 | 9.5 |
| 104702 (SS) OA Synovium-Backus | 8.3 | 7.9 |
| 117093 OA Cartilage Rep7 | 2.0 | 2.3 |
| 112672 OA Bone5 | 1.9 | 0.8 |
| 112673 OA Synovium5 | 0.3 | 1.2 |
| 112674 OA Synovial Fluid cells5 | 0.5 | 0.4 |
| 117100 OA Cartilage Rep14 | 0.4 | 0.3 |
| 112756 OA Bone9 | 100.0 | 100.0 |
| 112757 OA Synovium9 | 0.5 | 0.2 |
| 112758 OA Synovial Fluid Cells9 | 0.8 | 1.5 |
| 117125 RA Cartilage Rep2 | 1.0 | 0.6 |
| 113492 Bone2 RA | 2.8 | 3.6 |
| 113493 Synovium2 RA | 1.7 | 0.7 |
| 113494 Syn Fluid Cells RA | 0.9 | 2.1 |
| 113499 Cartilage4 RA | 2.1 | 1.8 |
| 113500 Bone4 RA | 1.8 | 2.5 |
| 113501 Synovium4 RA | 2.1 | 2.3 |
| 113502 Syn Fluid Cells4 RA | 1.0 | 0.8 |
| 113495 Cartilage3 RA | 2.5 | 2.6 |
| 113496 Bone3 RA | 2.0 | 2.1 |
| 113497 Synovium3 RA | 1.3 | 1.4 |
| 113498 Syn Fluid Cells3 RA | 2.9 | 3.2 |
| 117106 Normal Cartilage Rep20 | 0.1 | 0.7 |
| 113663 Bone3 Normal | 0.3 | 0.1 |
| 113664 Synovium3 Normal | 0.0 | 0.0 |
| 113665 Syn Fluid Cells3 Normal | 0.1 | 0.2 |
| 117107 Normal Cartilage Rep22 | 0.9 | 0.3 |
| 113667 Bone4 Normal | 0.4 | 0.7 |
| 113668 Synovium4 Normal | 1.0 | 1.1 |
| 113669 Syn Fluid Cells4 Normal | 1.0 | 0.7 |

Panel 1.3D Summary: Ag2100 Expression of the NOV3a gene is highest in cerebral cortex (CT=26.3). This gene is expressed at more moderate levels in other parts of the CNS including amygdala, cerebellum, hippocampus, substantia nigra, thalamus, spinal cord, and fetal brain. Expression of the NOV3a gene in other normal tissues was lower than that in brain, suggesting a specific function for this protein in the CNS. Thus, this gene may be useful as a marker to distinguish brain from other tissues. The NOV3a gene encodes a protein with homology to citron-kinase. Citron-kinase (Citron-K) has been proposed by in vitro studies to be a crucial effector of Rho in regulation of cytokinesis. Citron-K is essential for cytokinesis in vivo in specific neuronal precursors and may play a fundamental role in specific human malformative syndromes of the CNS. General inhibitors of the RHO/RAC-INTERACTING CITRON KINASE family disrupt endothelial tight junctions, suggesting that specific modulators of this brain-preferential family member could be useful in delivery of therapeutics across the blood brain barrier. These general inhibitors also influence intracellular calcium flux, which is a central component of many important neuronal processes, such as apoptosis, neurotransmitter release and signal transduction. Thus, modulators of NOV3a protein function may prove useful in the treatment of neurodegenerative disorders involving apoptosis, such as spinal muscular atrophy, Alzheimer's disease, Huntington's disease, Parkinson's disease, and others. Diseases involving neurotransmitters or signal transduction, such as schizophrenia, mania, stroke, epilepsy and depression may also benefit from agents that modulate the function of the NOV3a gene product.

The NOV3a gene also shows low expression in several metabolic tissues including adrenal gland (CT=32), pituitary gland (CT=32) and fetal heart (CT=34). Interestingly, this gene is expressed at higher levels in adult skeletal muscle (CT=37) compared to fetal skeletal muscle (CT=31) as well as in adult liver (CT=40) compared to fetal liver (CT=31). Thus, the NOV3A gene may be used to differentiate between the fetal and adult skeletal muscle and liver. Moreover, the therapeutic modulation of this gene, specifically its use in replacement type therapy through the administration of purified protein, might be beneficial in the treatment of diseases involving the degeneration of liver or skeletal muscle, such as muscular dystrophy.

Panel 2.2 Summary: Ag2100 Expression of the NOV3a gene is highest in a kidney cancer sample (CT=28). In addition, there are a number of normal tissue/cancer tissue pairs in which this gene is expressed at higher levels in the tumor than the normal matched tissue. Thus, expression of the NOV3a gene could be used to distinguish between cancerous tissue and normal tissue. In addition, therapeutic modulation of this gene product, through the use of small molecule drugs or antibodies, might be of benefit in the treatment of cancer.

Panel 3D Summary: Ag2100 Expression of the NOV3a gene is highest in a lung cancer cell line (CT=26). However, low to moderate expression is also seen in the majority of cancer cell lines on this panel, suggesting that this gene may play an important role in many cell types.

Panel 4D Summary: Ag2100 The NOV3a gene is highly induced in Ramos B cells treated with PMA and ionomycin, in non-transformed B cells treated with PWM and in PBMC treated with PWM. All three of these observations are consistent with this transcript being induced in B cells after activation. Upon activation, T cells also produce this transcript, PBMC treated with PHA (T cell mitogen) express the transcript as well as primary activated Th1 cells. Primary Tr1 and Th2 express the transcript to a lesser extent. Fibroblost and endothelial cell lines on this panel also express NOV3a gene, although at lower levels as compared to the activated lymphocytes.

Role in inflammation: The NOV3a gene product has homology to the RHO/RAC-interacting citron kinase. The citron kinase may play an important role in T cell activation, by regulating TCR-mediated T cell spreading, chemotaxis and other chemokine responses and in apoptosis. Since the protein encoded for by the NOV3a gene has high homology to this kinase, it too could contribute to T cell motility, activation and apoptosis. Likewise, this putative kinase may also be important in B cell motility, antigen receptor mediated activation and apoptosis.

Therapeutic function: Small molecule therapeutics designed against the protein encoded for by the NOV3a gene could reduce or inhibit inflammation. Anti-sense therapeutics that would block the translation of the transcript and protein production could also inhibit inflammatory processes. These types of therapeutics could be important in the treatment of diseases such as osteoarthritis. Likewise, these therapeutics could be important in the treatment of asthma, psoriasis, diabetes, and IBD, which require activated T cells, as well as diseases that involve B cell activation such as systemic lupus erythematosus.

AI_comprehensive_panel_v1.0 Summary: Ag2100 The NOV3A gene is highly expressed in bone isolated from 5 different-osteoarthritic (OA) patients, synovium in 3 out of 5 OA patients, but not in cartilege from OA patients nor in any tissues from rheumatoid arthritis (RA) patients or in control samples. Thus, small molecule therapeutics designed against the protein encoded for by the NOV3a gene could reduce or inhibit inflammation. Anti-sense therapeutics that would block the translation of the transcript and protein production could also inhibit inflammatory processes. These types of therapeutics could be important in the treatment of diseases such as osteoarthritis

References:

1. Di Cunto F., Imarisio S., Hirsch E., Broccoli V., Bulfone A., Migheli A., Atzori C., Turco E., Triolo R., Dotto G.P., Silengo L., Altruda F. (2000) Defective neurogenesis in citron kinase knockout mice by altered cytokinesis and massive apoptosis. Neuron 28:115-127.

Citron-kinase (Citron-K) has been proposed by in vitro studies as a crucial effector of Rho in regulation of cytokinesis. To further investigate in vivo its biologic functions, we have inactivated Citron-K gene in mice by homologous recombination. Citron-K−/− mice grow at slower rates, are severely ataxic, and die before adulthood as a consequence of fatal seizures. Their brains display defective neurogenesis, with depletion of specific neuronal populations. These abnormalities arise during development of the central nervous system due to altered cytokinesis and massive apoptosis. The results indicate that Citron-K is essential for cytokinesis in vivo but only in specific neuronal precursors. Moreover, they suggest a novel molecular mechanism for a subset of human malformative syndromes of the CNS.

PMID: 11086988

2. Jezior J. R., Brady J. D., Rosenstein D. I., McCammon K. A., Miner A. S., Ratz P. H. (2001)

Dependency of detrusor contractions on calcium sensitization and calcium entry through LOE-908-sensitive channels. Br. J. Pharmacol. 134:78-87.

The subcellular mechanisms regulating stimulus-contraction coupling in detrusor remain to be determined. Ca(2+)-free solutions, Ca(2+) channel blockers, cyclopiazonic acid (CPA), and RhoA kinase (ROK) inhibitors were used to test the hypothesis that Ca(2+) influx and Ca(2+) sensitization play primary roles. In rabbit detrusor, peak bethanechol (BE)-induced force was inhibited 90% by incubation for 3 min in a Ca(2+)-free solution. By comparison, a 20 min incubation of rabbit femoral artery in a Ca(2+)-free solution reduced receptor-induced force by only 5%. In detrusor, inhibition of sarcoplasmic reticular (SR) Ca(2+) release by 2APB, or depletion of SR Ca(2+) by CPA, inhibited BE-induced force by only 27%. The CPA-insensitive force was abolished by LaCl(3). By comparison, 2APB inhibited receptor-induced force in rabbit femoral artery by 71%. In the presence of the non-selective cation channel (NSCC) inhibitor, LOE-908, BE did not produce an increase in [Ca(2+)](i) but did produce weak increases in myosin phosphorylation and force. Inhibitors of ROK-induced Ca(2+) sensitization, HA-1077 and Y-27632, inhibited BE-induced force by approximately 50%, and in combination with LOE-908, nearly abolished force. These data suggest that two principal muscarinic receptor-stimulated detrusor contractile mechanisms include NSCC activation, that elevates [Ca(2+)](i) and ROK activation, that sensitizes cross bridges to Ca(2+).

PMID: 11522599

3. Walsh S. V., Hopkins A. M., Chen J., Narumiya S., Parkos C. A., Nusrat A. (2001) Rho kinase regulates tight junction function and is necessary for tight junction assembly in polarized intestinal epithelia. Gastroenterology 121:566-579.

Background & Aims: Tight junctions are crucial determinants of barrier function in polarized intestinal epithelia and are regulated by Rho guanosine triphosphatase. Rho kinase (ROCK) is a downstream effector of Rho. Methods: A specific inhibitor of ROCK, Y-27632, was used to examine the role of ROCK in the regulation of tight junctions in model intestinal (T84) cells by electrophysiologic, biochemical, morphologic, and molecular biologic approaches. Results: ROCK inhibition induced reorganization of apical F-actin structures and enhanced paracellular permeability but did not alter the distribution or detergent solubility of tight junction proteins. Confocal microscopy showed colocalization of a subpool of ROCK with the tight junction protein zonula occludens 1. Inhibition of ROCK function by a dominant negative mutant of ROCK also produced reorganization of apical F-actin structures without disruption of tight junctions. ROCK inhibition in calcium switch assays showed that ROCK is necessary for the assembly of tight and adherens junctions. Upon calcium repletion, occludin, zonula occludens 1, and E-cadherin failed to redistribute to the intercellularjunctions; assembly of the apical F-actin cytoskeleton was prevented; and barrier function failed to recover. Conclusions: It has been suggested that ROCK regulates intact tight junctions via its effects on the F-actin cytoskeleton. ROCK is also critical for assembly of the apical junctional proteins and the F-actin cytoskeleton organization during junctional formation.

PMID: 11522741

NOV4

Expression of gene NOV4 was assessed using the primer-probe sets Ag217, Ag850, and Ag1469, described in Tables 25, 26, and 27. Results from RTQ-PCR runs are shown in Tables 28, 29, 30, 31, 32, and 33.

TABLE 25

Probe Name Ag217

| | | | | Start | SEQ |
|---|---|---|---|---|---|
| Pri- | | | | Posi- | ID |
| mers | Sequences | TM | Length | tion | NO: |
| For- | 5′- | | 22 | 163 | 100 |
| ward | ATCTGTGCTGAGGCATGTTCCT- | | | | |
| | 3′ | | | | |
| Probe | FAM-5′- | | 23 | 192 | 101 |
| | ATCCTCCTCCCTCCCCGGCTCTC- | | | | |
| | 3′-TAMRA | | | | |
| Re- | 5′-CTGCATGGCTGGTGTGATG- | | 19 | 222 | 102 |
| verse | 3′ | | | | |

TABLE 26

Probe Name Ag850

| | | | | | |
|---|---|---|---|---|---|
| | | | | Start | SEQ ID |
| Primers | Sequences | TM | Length | Position | NO: |

| | | | | | |
|---|---|---|---|---|---|
| Forward | 5′-CCTTTCTTCTCTTCCTCCTCAA- | 59.1 | 22 | 25 | 103 |
| | 3′ | | | | |
| Probe | FAM-5′- | 70 | 23 | 71 | 104 |
| | CACCTGGCGAGTGCTCCTCTCTG-3′- | | | | |
| | TAMRA | | | | |
| Reverse | 5′-GGTGGATGGCGTTGTAGAG-3′ | 59.1 | 19 | 96 | 105 |

(Please note that there is a single base mismatch within forward primer that is not predicted to affect binding)

TABLE 27
Probe Name Ag1469

| | | | | | |
|---|---|---|---|---|---|
| | | | | Start | SEQ |
| Primers | Sequences | TM | Length | Position | ID NO: |

| | | | | | |
|---|---|---|---|---|---|
| Forward | 5′-CGTACGTCTTCCATGATGAGTT-3′ | 59.1 | 22 | 644 | 106 |
| Probe | TET-5′-CGTGGCCTCGATGATTAAGATCCCTT-3′-TAMRA | 69.8 | 26 | 666 | 107 |
| Reverse | 5′-AAGTCAGGGATGATGGTGAAG-3′ | 59 | 21 | 699 | 108 |

TABLE 28
Panel 1

| | Relative | | Relative |
|---|---|---|---|
| | Expression (%) | | Expression (%) |
| Tissue Name | tm303f | Tissue Name | tm303f |
| Endothelial cells | 0.0 | Renal ca. 786-0 | 0.0 |
| Endothelial cells (treated) | 0.0 | Renal ca. A498 | 0.0 |
| Pancreas | 0.0 | Renal ca. RXF 393 | 0.0 |
| Pancreatic ca. CAPAN 2 | 5.4 | Renal ca. ACHN | 0.0 |
| Adrenal gland | 0.0 | Renal ca. UO-31 | 0.0 |
| Thyroid | 0.0 | Renal ca. TK-10 | 0.0 |
| Salavary gland | 0.0 | Liver | 0.0 |
| Pituitary gland | 0.0 | Liver (fetal) | 0.0 |
| Brain (fetal) | 0.0 | Liver ca. (hepatoblast) HepG2 | 0.0 |
| Brain (whole) | 0.0 | Lung | 0.0 |
| Brain (amygdala) | 0.0 | Lung (fetal) | 0.0 |
| Brain (cerebellum) | 0.0 | Lung ca. (small cell) LX-1 | 3.1 |
| Brain (hippocampus) | 0.0 | Lung ca. (small cell) NCI-H69 | 0.2 |
| Brain (substantia nigra) | 0.0 | Lung ca. (s.cell var.) SHP-77 | 0.0 |
| Brain (thalamus) | 0.0 | Lung ca. (large cell)NCI-H460 | 0.0 |
| Brain (hypothalamus) | 0.0 | Lung ca. (non-sm. cell) A549 | 2.2 |
| Spinal cord | 0.0 | Lung ca. (non-s.cell) NCI-H23 | 0.0 |
| CNS ca. (glio/astro) U87-MG | 0.0 | Lung ca. (non-s.cell) HOP-62 | 0.0 |
| CNS ca. (glio/astro) U-118-MG | 0.0 | Lung ca. (non-s.cl) NCI-H522 | 0.0 |
| CNS ca. (astro) SW1783 | 0.3 | Lung ca. (squam.) SW 900 | 0.6 |
| CNS ca.* (neuro; met) SK-N- | 6.7 | Lung ca. (squam.) NCI-H596 | 0.2 |
| AS | | | |
| CNS ca. (astro) SF-539 | 0.0 | Mammary gland | 0.0 |
| CNS ca. (astro) SNB-75 | 0.0 | Breast ca.* (pl. effusion) MCF-7 | 0.0 |
| CNS ca. (glio) SNB-19 | 0.0 | Breast ca.* (pl.ef) MDA-MB- | 0.0 |
| | | 231 | |
| CNS ca. (glio) U251 | 0.6 | Breast ca.* (pl. effusion) T47D | 0.0 |
| CNS ca. (glio) SF-295 | 15.3 | Breast ca. BT-549 | 0.0 |
| Heart | 0.0 | Breast ca. MDA-N | 0.0 |
| Skeletal muscle | 0.0 | Ovary | 0.0 |
| Bone marrow | 0.0 | Ovarian ca. OVCAR-3 | 0.0 |
| Thymus | 0.0 | Ovarian ca. OVCAR-4 | 0.0 |
| Spleen | 0.0 | Ovarian ca. OVCAR-5 | 0.1 |
| Lymph node | 0.0 | Ovarian ca. OVCAR-8 | 0.0 |
| Colon (ascending) | 0.0 | Ovarian ca. IGROV-1 | 0.0 |
| Stomach | 0.0 | Ovarian ca.* (ascites) SK-OV-3 | 0.0 |
| Small intestine | 0.0 | Uterus | 7.4 |
| Colon ca. SW480 | 2.4 | Placenta | 100.0 |
| Colon ca.* (SW480 | 4.1 | Prostate | 0.0 |
| met)SW620 | | | |
| Colon ca. HT29 | 0.0 | Prostate ca.* (bone met)PC-3 | 0.0 |
| Colon ca. HCT-116 | 0.0 | Testis | 4.2 |
| Colon ca. CaCo-2 | 0.0 | Melanoma Hs688(A).T | 0.0 |
| Colon ca. HCT-15 | 0.0 | Melanoma* (met) Hs688(B).T | 0.0 |
| Colon ca. HCC-2998 | 0.0 | Melanoma UACC-62 | 0.0 |
| Gastric ca.* (liver met) NCI- | 45.4 | Melanoma M14 | 0.0 |
| N87 | | | |
| Bladder | 0.0 | Melanoma LOX IMVI | 0.0 |
| Trachea | 0.0 | Melanoma* (met) SK-MEL-5 | 0.0 |
| Kidney | 4.1 | Melanoma SK-MEL-28 | 0.0 |
| Kidney (fetal) | 0.0 | | |

TABLE 29
Panel 1.3D

| | Relative | | Relative |
|---|---|---|---|
| | Expression (%) | | Expression (%) |
| | 1.3Dtm2782t_{—} | | 1.3Dtm2782t_{—} |
| Tissue Name | ag1469 | Tissue Name | ag1469 |
| Liver adenocarcinoma | 0.0 | Kidney (fetal) | 1.2 |
| Pancreas | 0.0 | Renal ca. 786-0 | 0.0 |
| Pancreatic ca. CAPAN 2 | 0.0 | Renal ca. A498 | 4.1 |
| Adrenal gland | 0.6 | Renal ca. RXF 393 | 2.5 |
| Thyroid | 0.6 | Renal ca. ACHN | 0.0 |
| Salivary gland | 0.0 | Renal ca. UO-31 | 0.4 |
| Pituitary gland | 0.9 | Renal ca. TK-10 | 0.2 |
| Brain (fetal) | 14.5 | Liver | 0.0 |
| Brain (whole) | 9.1 | Liver (fetal) | 0.1 |
| Brain (amygdala) | 8.2 | Liver ca. (hepatoblast) HepG2 | 0.0 |
| Brain (cerebellum) | 0.9 | Lung | 0.0 |
| Brain (hippocampus) | 14.9 | Lung (fetal) | 1.1 |
| Brain (substantia nigra) | 0.8 | Lung ca. (small cell) LX-1 | 0.0 |
| Brain (thalamus) | 3.6 | Lung ca. (small cell) NCI-H69 | 0.0 |
| Cerebral Cortex | 100.0 | Lung ca. (s.cell var.) SHP-77 | 2.1 |
| Spinal cord | 0.6 | Lung ca. (large cell)NCI-H460 | 0.0 |
| CNS ca. (glio/astro) U87-MG | 0.9 | Lung ca. (non-sm. cell) A549 | 0.0 |
| CNS ca. (glio/astro) U-118-MG | 15.1 | Lung ca. (non-s.cell) NCI-H23 | 0.2 |
| CNS ca. (astro) SW1783 | 0.5 | Lung ca. (non-s.cell) HOP-62 | 0.0 |
| CNS ca.* (neuro; met) SK-N- | 1.3 | Lung ca. (non-s.cl) NCI-H522 | 0.0 |
| AS | | | |
| CNS ca. (astro) SF-539 | 0.0 | Lung ca. (squam.) SW 900 | 0.0 |
| CNS ca. (astro) SNB-75 | 10.2 | Lung ca. (squam.) NCI-H596 | 0.0 |
| CNS ca. (glio) SNB-19 | 16.3 | Mammary gland | 8.9 |
| CNS ca. (glio) U251 | 1.7 | Breast ca.* (pl. effusion) MCF-7 | 12.9 |
| CNS ca. (glio) SF-295 | 0.3 | Breast ca.* (pl.ef) MDA-MB- | 0.6 |
| | | 231 | |
| Heart (fetal) | 81.2 | Breast ca.* (pl. effusion) T47D | 0.0 |
| Heart | 2.3 | Breast ca. BT-549 | 1.1 |
| Fetal Skeletal | 16.3 | Breast ca. MDA-N | 0.0 |
| Skeletal muscle | 0.1 | Ovary | 9.8 |
| Bone marrow | 0.3 | Ovarian ca. OVCAR-3 | 0.1 |
| Thymus | 0.4 | Ovarian ca. OVCAR-4 | 0.0 |
| Spleen | 0.6 | Ovarian ca. OVCAR-5 | 0.0 |
| Lymph node | 1.9 | Ovarian ca. OVCAR-8 | 0.0 |
| Colorectal | 4.4 | Ovarian ca. IGROV-1 | 4.5 |
| Stomach | 1.4 | Ovarian ca.* (ascites) SK-OV-3 | 0.6 |
| Small intestine | 0.5 | Uterus | 0.3 |
| Colon ca. SW480 | 0.0 | Placenta | 2.4 |
| Colon ca.* (SW480 met)SW620 | 0.0 | Prostate | 0.0 |
| Colon ca. HT29 | 0.0 | Prostate ca.* (bone met)PC-3 | 0.4 |
| Colon ca. HCT-116 | 0.0 | Testis | 1.7 |
| Colon ca. CaCo-2 | 0.0 | Melanoma Hs688(A).T | 2.5 |
| 83219 CC Well to Mod Diff | 6.5 | Melanoma* (met) Hs688(B).T | 6.8 |
| (ODO3866) | | | |
| Colon ca. HCC-2998 | 0.0 | Melanoma UACC-62 | 0.0 |
| Gastric ca.* (liver met) NCI- | 0.0 | Melanoma M14 | 0.0 |
| N87 | | | |
| Bladder | 0.4 | Melanoma LOX IMVI | 0.0 |
| Trachea | 0.0 | Melanoma* (met) SK-MEL-5 | 0.4 |
| Kidney | 0.1 | Adipose | 10.6 |

TABLE 30
General_screening_panel_v1.4

| | Relative | Relative |
|---|---|---|
| | Expression (%) | Expression (%) |
| | 1.4tm7173f_{—} | 1.4tm7186f_{—} |
| Tissue Name | ag217_b2 | ag850_b2 |
| D6005-01_Human adipose | 0.3 | 0.2 |
| 112193_Metastatic melanoma | 0.0 | 0.0 |
| 112192_Metastatic melanoma | 0.4 | 0.2 |
| 95280_Epidermis (metastatic | 0.0 | 0.2 |
| melanoma) | | |
| 95279_Epidermis (metastatic | 1.0 | 0.6 |
| melanoma) | | |
| Melanoma (met)_SK-MEL-5 | 0.0 | 0.0 |
| 112196_Tongue (oncology) | 0.8 | 0.4 |
| 113461_Testis Pool | 1.0 | 0.5 |
| Prostate ca.(bone met)_PC-3 | 0.0 | 0.0 |
| 113455_Prostate Pool | 1.0 | 0.7 |
| 103396_Placenta | 33.5 | 31.8 |
| 113463_Uterus Pool | 0.1 | 0.0 |
| Ovarian carcinoma_OVCAR-3 | 0.1 | 0.1 |
| Ovarian carcinoma(ascites)_SK-OV-3 | 0.1 | 0.1 |
| 95297_Adenocarcinoma (ovary) | 0.1 | 0.0 |
| Ovarian carcinoma_OVCAR-5 | 4.4 | 4.9 |
| Ovarian carcinoma_IGROV-1 | 0.8 | 0.2 |
| Ovarian carcinoma_OVCAR-8 | 7.2 | 3.0 |
| 103368_Ovary | 0.5 | 0.6 |
| MCF7_breast carcinoma(pleural | 0.0 | 0.0 |
| effusion) | | |
| Breast ca. (pleural effusion)_MDA- | 0.4 | 0.0 |
| MB-231 | | |
| 112189_ductal cell carcinoma(breast) | 9.2 | 11.4 |
| Breast ca. (pleural effusion)_T47D | 14.5 | 4.5 |
| Breast carcinoma_MDA-N | 0.0 | 0.0 |
| 113452_Breast Pool | 2.6 | 1.4 |
| 103398_Trachea | 0.5 | 0.4 |
| 112354_lung | 0.1 | 0.0 |
| 103374_Fetal Lung | 0.2 | 0.4 |
| 94921_Small cell carcinoma of | 0.7 | 0.4 |
| the lung | | |
| Lung ca.(small cell)_LX-1 | 18.4 | 21.2 |
| 94919_Small cell carcinoma of | 1.8 | 0.8 |
| the lung | | |
| Lung ca.(s.cell var.)_SHP-77 | 1.8 | 1.0 |
| 95268_Lung (Large cell carcinoma) | 22.3 | 24.7 |
| 94920_Small cell carcinoma of | 0.0 | 0.1 |
| the lung | | |
| Lung ca.(non-s.cell)_NCI-H23 | 6.0 | 3.5 |
| Lung ca.(large cell)_NCI-H460 | 0.6 | 0.3 |
| Lung ca.(non-s.cell)_HOP-62 | 3.7 | 1.2 |
| Lung ca.(non-s.cl)_NCI-H522 | 0.0 | 0.0 |
| 103392_Liver | 0.0 | 0.0 |
| 103393_Fetal Liver | 1.0 | 1.1 |
| Liver ca.(hepatoblast)_HepG2 | 0.4 | 0.2 |
| 113465_Kidney Pool | 1.8 | 0.8 |
| 103373_Fetal Kidney | 2.9 | 2.5 |
| Renal ca._786-0 | 0.1 | 0.0 |
| 112188_renal cell carcinoma | 0.3 | 0.2 |
| Renal ca._ACHN | 1.2 | 1.9 |
| 112190_Renal cell carcinoma | 0.0 | 0.0 |
| Renal ca._TK-10 | 0.7 | 0.2 |
| Bladder | 1.0 | 0.5 |
| Gastric ca.(liver met)_NCI-N87 | 87.0 | 64.6 |
| 112197_Stomach | 0.0 | 0.0 |
| 94938_Colon Adenocarcinoma | 0.0 | 0.0 |
| Colon ca._SW480 | 69.8 | 58.3 |
| Colon ca.(SW480 met)_SW620 | 29.8 | 22.4 |
| Colon ca._HT29 | 1.7 | 1.0 |
| Colon ca._HCT-116 | 0.0 | 0.0 |
| Colon ca._CaCo-2 | 1.0 | 0.7 |
| 83219_CC Well to Mod Diff | 0.6 | 0.2 |
| (ODO3866) | | |
| 94936_Colon Adenocarcinoma | 0.9 | 0.6 |
| 94930_Colon | 0.7 | 0.3 |
| 94935_Colon Adenocarcinoma | 0.0 | 0.0 |
| 113468_Colon Pool | 0.7 | 0.5 |
| 113457_Small Intestine Pool | 6.3 | 2.8 |
| 113460_Stomach Pool | 1.1 | 2.1 |
| 113467_Bone Marrow Pool | 1.3 | 0.9 |
| 103371_Fetal Heart | 0.0 | 0.0 |
| 113451_Heart Pool | 0.2 | 0.0 |
| 113466_Lymph Node Pool | 2.1 | 3.0 |
| 103372_Fetal Skeletal Muscle | 0.1 | 0.0 |
| 113456_Skeletal Muscle Pool | 0.0 | 0.1 |
| 113459_Spleen Pool | 0.0 | 0.0 |
| 113462_Thymus Pool | 6.7 | 3.3 |
| CNS ca. (glio/astro)_U87-MG | 0.7 | 0.6 |
| CNS ca. (glio/astro)_U-118-MG | 0.3 | 0.2 |
| CNS ca. (neuro; met)_SK-N-AS | 21.0 | 16.9 |
| 95264_Brain astrocytoma | 2.2 | 2.4 |
| CNS ca. (astro)_SNB-75 | 0.0 | 0.0 |
| CNS ca. (glio)_SNB-19 | 0.5 | 0.1 |
| CNS ca. (glio)_SF-295 | 100.0 | 100.0 |
| 113447_Brain (Amygdala) Pool | 0.0 | 0.0 |
| 103382_Brain (cerebellum) | 0.2 | 0.2 |
| 64019-1_brain(fetal) | 0.8 | 0.7 |
| 113448_Brain (Hippocampus) Pool | 0.0 | 0.1 |
| 113464_Cerebral Cortex Pool | 0.2 | 0.2 |
| 113449_Brain (Substantia nigra) Pool | 0.0 | 0.0 |
| 113450_Brain (Thalamus) Pool | 0.0 | 0.0 |
| 103384_Brain (whole) | 0.3 | 0.2 |
| 113458_Spinal Cord Pool | 0.4 | 0.2 |
| 103375_Adrenal Gland | 0.4 | 0.3 |
| 113454_Pituitary gland Pool | 0.8 | 0.5 |
| 103397_Salivary Gland | 0.0 | 0.0 |
| 103369_Thyroid (female) | 0.4 | 0.1 |
| Pancreatic ca._CAPAN2 | 34.6 | 25.5 |
| 113453_Pancreas Pool | 5.7 | 4.5 |

TABLE 31
Panel 2D

| | Relative Expression (%) | | |
|---|---|---|---|
| | 2Dtm2759t_{—} | 2Dtm3205t_{—} | 2Dtm3211t_{—} |
| Tissue Name | ag1469 | ag1469 | ag1469 |
| Normal Colon GENPAK 061003 | 21.2 | 24.0 | 25.7 |
| 83219 CC Well to Mod Diff (ODO3866) | 4.9 | 6.7 | 6.3 |
| 83220 CC NAT (ODO3866) | 2.4 | 7.6 | 5.7 |
| 83221 CC Gr. 2 rectosigmoid (ODO3868) | 4.4 | 6.5 | 4.5 |
| 83222 CC NAT (ODO3868) | 13.1 | 14.5 | 11.2 |
| 83235 CC Mod Diff (ODO3920) | 2.2 | 2.4 | 2.6 |
| 83236 CC NAT (ODO3920) | 6.3 | 6.5 | 4.9 |
| 83237 CC Gr. 2 ascend colon (ODO3921) | 5.7 | 9.3 | 6.4 |
| 83238 CC NAT (ODO3921) | 8.4 | 11.3 | 8.6 |
| 83241 CC from Partial Hepatectomy | 5.2 | 4.5 | 3.2 |
| (ODO4309) | | | |
| 83242 Liver NAT (ODO4309) | 2.0 | 3.4 | 2.7 |
| 87472 Colon mets to lung (OD04451-01) | 4.5 | 4.3 | 4.0 |
| 87473 Lung NAT (OD04451-02) | 3.0 | 3.0 | 3.1 |
| Normal Prostate Clontech A+ 6546-1 | 0.2 | 0.3 | 0.5 |
| 84140 Prostate Cancer (OD04410) | 2.9 | 5.4 | 2.8 |
| 84141 Prostate NAT (OD04410) | 9.9 | 13.5 | 10.2 |
| 87073 Prostate Cancer (OD04720-01) | 6.0 | 6.8 | 4.6 |
| 87074 Prostate NAT (OD04720-02) | 11.3 | 8.9 | 4.2 |
| Normal Lung GENPAK 061010 | 18.6 | 19.2 | 12.7 |
| 83239 Lung Met to Muscle (ODO4286) | 0.0 | 1.2 | 1.0 |
| 83240 Muscle NAT (ODO4286) | 4.7 | 6.0 | 4.4 |
| 84136 Lung Malignant Cancer (OD03126) | 4.8 | 8.5 | 5.8 |
| 84137 Lung NAT (OD03126) | 8.5 | 15.9 | 8.1 |
| 84871 Lung Cancer (OD04404) | 7.9 | 7.6 | 4.9 |
| 84872 Lung NAT (OD04404) | 16.5 | 23.0 | 14.5 |
| 84875 Lung Cancer (OD04565) | 4.9 | 3.1 | 3.4 |
| 84876 Lung NAT (OD04565) | 3.6 | 9.5 | 2.9 |
| 85950 Lung Cancer (OD04237-01) | 1.4 | 4.1 | 2.1 |
| 85970 Lung NAT (OD04237-02) | 8.4 | 9.3 | 7.5 |
| 83255 Ocular Mel Met to Liver (ODO4310) | 0.6 | 0.5 | 0.2 |
| 83256 Liver NAT (ODO4310) | 1.0 | 1.0 | 0.7 |
| 84139 Melanoma Mets to Lung (OD04321) | 0.2 | 0.2 | 0.2 |
| 84138 Lung NAT (OD04321) | 6.7 | 9.4 | 5.4 |
| Normal Kidney GENPAK 061008 | 11.7 | 17.3 | 12.4 |
| 83786 Kidney Ca, Nuclear grade 2 (OD04338) | 3.0 | 3.8 | 4.1 |
| 83787 Kidney NAT (OD04338) | 13.8 | 19.5 | 14.3 |
| 83788 Kidney Ca Nuclear grade ½ (OD04339) | 16.8 | 19.1 | 15.0 |
| 83789 Kidney NAT (OD04339) | 4.9 | 4.6 | 4.6 |
| 83790 Kidney Ca, Clear cell type (OD04340) | 0.9 | 1.6 | 0.8 |
| 83791 Kidney NAT (OD04340) | 11.5 | 18.3 | 14.1 |
| 83792 Kidney Ca, Nuclear grade 3 (OD04348) | 0.6 | 1.2 | 0.4 |
| 83793 Kidney NAT (OD04348) | 8.5 | 15.4 | 10.9 |
| 87474 Kidney Cancer (OD04622-01) | 2.9 | 4.3 | 3.4 |
| 87475 Kidney NAT (OD04622-03) | 1.9 | 4.5 | 3.9 |
| 85973 Kidney Cancer (OD04450-01) | 0.3 | 0.0 | 0.4 |
| 85974 Kidney NAT (OD04450-03) | 5.4 | 7.8 | 4.8 |
| Kidney Cancer Clontech 8120607 | 1.4 | 2.3 | 1.7 |
| Kidney NAT Clontech 8120608 | 3.6 | 4.2 | 3.1 |
| Kidney Cancer Clontech 8120613 | 0.6 | 0.3 | 0.7 |
| Kidney NAT Clontech 8120614 | 59.9 | 5.4 | 4.9 |
| Kidney Cancer Clontech 9010320 | 10.7 | 9.2 | 11.7 |
| Kidney NAT Clontech 9010321 | 8.8 | 8.1 | 14.2 |
| Normal Uterus GENPAK 061018 | 15.0 | 15.3 | 14.2 |
| Uterus Cancer GENPAK 064011 | 9.3 | 10.2 | 13.1 |
| Normal Thyroid Clontech A+ 6570-1 | 3.1 | 3.6 | 2.6 |
| Thyroid Cancer GENPAK 064010 | 20.7 | 30.1 | 23.2 |
| Thyroid Cancer INVITROGEN A302152 | 9.4 | 20.9 | 7.3 |
| Thyroid NAT INVITROGEN A302153 | 1.6 | 4.0 | 4.0 |
| Normal Breast GENPAK 061019 | 45.4 | 62.4 | 41.8 |
| 84877 Breast Cancer (OD04566) | 4.5 | 5.7 | 3.0 |
| 85975 Breast Cancer (OD04590-01) | 10.5 | 17.8 | 14.9 |
| 85976 Breast Cancer Mets (OD04590-03) | 44.4 | 51.4 | 35.1 |
| 87070 Breast Cancer Metastasis (OD04655-05) | 11.0 | 14.2 | 12.2 |
| GENPAK Breast Cancer 064006 | 7.1 | 8.3 | 6.0 |
| Breast Cancer Res. Gen. 1024 | 38.2 | 37.1 | 43.8 |
| Breast Cancer Clontech 9100266 | 5.4 | 4.8 | 4.1 |
| Breast NAT Clontech 9100265 | 4.9 | 10.3 | 6.8 |
| Breast Cancer INVITROGEN A209073 | 11.7 | 24.7 | 13.5 |
| Breast NAT INVITROGEN A2090734 | 20.9 | 23.3 | 16.0 |
| Normal Liver GENPAK 061009 | 8.5 | 12.6 | 5.5 |
| Liver Cancer GENPAK 064003 | 0.4 | 0.9 | 0.5 |
| Liver Cancer Research Genetics RNA 1025 | 3.9 | 4.2 | 2.0 |
| Liver Cancer Research Genetics RNA 1026 | 1.7 | 4.2 | 3.7 |
| Paired Liver Cancer Tissue Research Genetics | 2.8 | 2.8 | 2.8 |
| RNA 6004-T | | | |
| Paired Liver Tissue Research Genetics RNA | 0.8 | 1.3 | 0.8 |
| 6004-N | | | |
| Paired Liver Cancer Tissue Research Genetics | 3.7 | 3.0 | 1.1 |
| RNA 6005-T | | | |
| Paired Liver Tissue Research Genetics RNA | 1.2 | 2.6 | 1.6 |
| 6005-N | | | |
| Normal Bladder GENPAK 061001 | 5.8 | 9.7 | 11.0 |
| Bladder Cancer Research Genetics RNA 1023 | 2.6 | 4.3 | 3.6 |
| Bladder Cancer INVITROGEN A302173 | 2.3 | 1.2 | 1.8 |
| 87071 Bladder Cancer (OD04718-01) | 0.7 | 3.6 | 1.8 |
| 87072 Bladder Normal Adjacent (OD04718-03) | 12.7 | 16.6 | 15.1 |
| Normal Ovary Res. Gen. | 13.2 | 15.9 | 12.2 |
| Ovarian Cancer GENPAK 064008 | 100.0 | 100.0 | 100.0 |
| 87492 Ovary Cancer (OD04768-07) | 0.6 | 0.7 | 0.6 |
| 87493 Ovary NAT (OD04768-08) | 18.8 | 26.4 | 23.7 |
| Normal Stomach GENPAK 061017 | 17.2 | 19.8 | 20.7 |
| Gastric Cancer Clontech 9060358 | 4.6 | 7.0 | 4.2 |
| NAT Stomach Clontech 9060359 | 2.7 | 3.3 | 3.8 |
| Gastric Cancer Clontech 9060395 | 17.0 | 17.3 | 14.7 |
| NAT Stomach Clontech 9060394 | 9.9 | 15.1 | 9.3 |
| Gastric Cancer Clontech 9060397 | 8.5 | 11.1 | 10.1 |
| NAT Stomach Clontech 9060396 | 2.1 | 2.8 | 2.8 |
| Gastric Cancer GENPAK 064005 | 8.2 | 13.0 | 9.2 |

TABLE 32
Panel 4D

| | | | Relative |
|---|---|---|---|
| | | | Expression |
| | Relative Expression (%) | | (%) |
| | 4dx4tm5043f_{—} | 4dx4tm5056f_{—} | 4Dtm2436t_{—} |
| Tissue Name | ag217_b1 | ag217_b1 | ag1469 |
| 93768_Secondary Th1_anti-CD28/anti-CD3 | 0.0 | 1.2 | 0.0 |
| 93769_Secondary Th2_anti-CD28/anti-CD3 | 0.0 | 0.0 | 5.5 |
| 93770_Secondary Tr1_anti-CD28/anti-CD3 | 2.6 | 0.0 | 1.6 |
| 93573_Secondary Th1_resting day 4-6 in IL-2 | 1.8 | 0.0 | 3.3 |
| 93572_Secondary Th2_resting day 4-6 in IL-2 | 0.0 | 0.0 | 23.0 |
| 93571_Secondary Tr1_resting day 4-6 in IL-2 | 7.0 | 1.6 | 12.6 |
| 93568_primary Th1_anti-CD28/anti-CD3 | 0.0 | 0.0 | 4.3 |
| 93569_primary Th2_anti-CD28/anti-CD3 | 0.0 | 0.0 | 22.2 |
| 93570_primary Tr1_anti-CD28/anti-CD3 | 0.8 | 0.0 | 12.6 |
| 93565_primary Th1_resting dy 4-6 in IL-2 | 0.0 | 0.0 | 33.7 |
| 93566_primary Th2_resting dy 4-6 in IL-2 | 0.0 | 0.0 | 45.7 |
| 93567_primary Tr1_resting dy 4-6 in IL-2 | 0.0 | 1.3 | 74.7 |
| 93351_CD45RA CD4 lymphocyte_anti- | 8.1 | 9.2 | 0.9 |
| CD28/anti-CD3 | | | |
| 93352_CD45RO CD4 lymphocyte_anti- | 0.0 | 0.0 | 0.0 |
| CD28/anti-CD3 | | | |
| 93251_CD8 Lymphocytes_anti-CD28/anti-CD3 | 0.0 | 0.0 | 2.4 |
| 93353_chronic CD8 Lymphocytes 2ry_resting | 0.0 | 0.0 | 0.7 |
| dy 4-6 in IL-2 | | | |
| 93574_chronic CD8 Lymphocytes | 2.1 | 2.5 | 0.3 |
| 2ry_activated CD3/CD28 | | | |
| 93354_CD4_none | 0.0 | 0.0 | 3.0 |
| 93252_Secondary Th1/Th2/Tr1_anti-CD95 | 0.0 | 0.0 | 5.9 |
| CH11 | | | |
| 93103_LAK cells_resting | 0.0 | 0.0 | 6.0 |
| 93788_LAK cells_IL-2 | 0.0 | 0.0 | 1.8 |
| 93787_LAK cells_IL-2 + IL-12 | 0.0 | 0.0 | 11.0 |
| 93789_LAK cells_IL-2 + IFN gamma | 0.0 | 0.0 | 11.9 |
| 93790_LAK cells_IL-2 + IL-18 | 2.2 | 0.0 | 9.8 |
| 93104_LAK cells_PMA/ionomycin and IL-18 | 0.0 | 0.0 | 2.8 |
| 93578_NK Cells IL-2_resting | 0.0 | 0.0 | 13.7 |
| 93109_Mixed Lymphocyte Reaction_Two Way | 0.0 | 0.0 | 6.0 |
| MLR | | | |
| 93110_Mixed Lymphocyte Reaction_Two Way | 0.0 | 0.0 | 1.3 |
| MLR | | | |
| 93111_Mixed Lymphocyte Reaction_Two Way | 0.0 | 1.5 | 0.6 |
| MLR | | | |
| 93112_Mononuclear Cells (PBMCs)_resting | 0.0 | 0.0 | 1.6 |
| 93113_Mononuclear Cells (PBMCs)_PWM | 0.0 | 0.0 | 5.1 |
| 93114_Mononuclear Cells (PBMCs)_PHA-L | 0.0 | 0.0 | 6.2 |
| 93249_Ramos (B cell)_none | 0.0 | 0.0 | 0.0 |
| 93250_Ramos (B cell)_ionomycin | 0.0 | 1.5 | 0.0 |
| 93349_B lymphocytes_PWM | 0.0 | 3.4 | 0.0 |
| 93350_B lymphoytes_CD40L and IL-4 | 0.0 | 1.7 | 2.9 |
| 92665_EOL-1 (Eosinophil)_dbcAMP | 0.0 | 0.0 | 0.0 |
| differentiated | | | |
| 93248_EOL-1 | 0.0 | 0.0 | 0.0 |
| (Eosinophil)_dbcAMP/PMAionomycin | | | |
| 93356_Dendritic Cells_none | 0.0 | 0.0 | 0.7 |
| 93355_Dendritic Cells_LPS 100 ng/ml | 1.9 | 1.4 | 0.0 |
| 93775_Dendritic Cells_anti-CD40 | 0.0 | 0.0 | 0.0 |
| 93774_Monocytes_resting | 0.0 | 0.0 | 0.2 |
| 93776_Monocytes_LPS 50 ng/ml | 0.0 | 0.0 | 0.4 |
| 93581_Macrophages_resting | 0.0 | 1.5 | 0.0 |
| 93582_Macrophages_LPS 100 ng/ml | 6.4 | 0.0 | 0.0 |
| 93098_HUVEC (Endothelial)_none | 0.0 | 0.0 | 22.4 |
| 93099_HUVEC (Endothelial)_starved | 0.0 | 0.0 | 45.7 |
| 93100_HUVEC (Endothelial)_IL-1b | 0.0 | 0.0 | 4.1 |
| 93779_HUVEC (Endothelial)_IFN gamma | 0.0 | 0.0 | 38.7 |
| 93102_HUVEC (Endothelial)_TNF alpha + IFN | 0.0 | 0.0 | 4.9 |
| gamma | | | |
| 93101_HUVEC (Endothelial)_TNF alpha + IL4 | 0.0 | 0.0 | 6.4 |
| 93781_HUVEC (Endothelial)_IL-11 | 0.0 | 2.8 | 16.6 |
| 93583_Lung Microvascular Endothelial | 0.0 | 1.1 | 92.7 |
| Cells_none | | | |
| 93584_Lung Microvascular Endothelial | 0.0 | 0.0 | 25.5 |
| Cells_TNFa (4 ng/ml) and IL1b (1 ng/ml) | | | |
| 92662_Microvascular Dermal | 3.7 | 0.0 | 100.0 |
| endothelium_none | | | |
| 92663_Microsvasular Dermal | 0.0 | 0.0 | 20.7 |
| endothelium_TNFa (4 ng/ml) and IL1b (1 ng/ml) | | | |
| 93773_Bronchial epithelium_TNFa (4 ng/ml) | 3.9 | 3.2 | 0.0 |
| and IL1b (1 ng/ml)** | | | |
| 93347_Small Airway Epithelium_none | 6.2 | 4.5 | 0.2 |
| 93348_Small Airway Epithelium_TNFa (4 ng/ml) | 11.2 | 9.7 | 1.6 |
| and IL1b (1 ng/ml) | | | |
| 92668_Coronery Artery SMC_resting | 5.8 | 5.9 | 3.3 |
| 92669_Coronery Artery SMC_TNFa (4 ng/ml) | 4.2 | 1.9 | 2.4 |
| and IL1b (1 ng/ml) | | | |
| 93107_astrocytes_resting | 61.1 | 41.5 | 27.2 |
| 93108_astrocytes_TNFa (4 ng/ml) and IL1b (1 ng/ml) | 78.6 | 69.9 | 10.4 |
| 92666_KU-812 (Basophil)_resting | 0.0 | 0.0 | 0.3 |
| 92667_KU-812 (Basophil)_PMA/ionoycin | 0.0 | 0.0 | 1.7 |
| 93579_CCD1106 (Keratinocytes)_none | 0.0 | 0.0 | 0.0 |
| 93580_CCD1106 (Keratinocytes)_TNFa and | 0.0 | 0.0 | 0.0 |
| IFNg** | | | |
| 93791_Liver Cirrhosis | 10.9 | 5.4 | 4.0 |
| 93792_Lupus Kidney | 0.0 | 2.3 | 10.4 |
| 93577_NCI-H292 | 11.6 | 2.8 | 0.0 |
| 93358_NCI-H292_IL-4 | 2.9 | 3.3 | 0.0 |
| 93360_NCI-H292_IL-9 | 2.3 | 1.8 | 0.0 |
| 93359_NCI-H292_IL-13 | 2.8 | 0.0 | 0.0 |
| 93357_NCI-H292_IFN gamma | 3.8 | 0.0 | 0.3 |
| 93777_HPAEC_- | 0.0 | 0.0 | 57.8 |
| 93778_HPAEC_IL-1beta/TNA alpha | 0.0 | 0.0 | 8.8 |
| 93254_Normal Human Lung Fibroblast_none | 0.0 | 0.0 | 7.5 |
| 93253_Normal Human Lung Fibroblast_TNFa | 0.0 | 0.0 | 6.7 |
| (4 ng/ml) and IL-1b (1 ng/ml) | | | |
| 93257_Normal Human Lung Fibroblast_IL-4 | 0.0 | 0.0 | 13.8 |
| 93256_Normal Human Lung Fibroblast_IL-9 | 0.0 | 0.0 | 7.2 |
| 93255_Normal Human Lung Fibroblast_IL-13 | 0.0 | 0.0 | 20.6 |
| 93258_Normal Human Lung Fibroblast_IFN | 0.0 | 1.3 | 34.6 |
| gamma | | | |
| 93106_Dermal Fibroblasts CCD1070_resting | 40.7 | 30.0 | 1.5 |
| 93361_Dermal Fibroblasts CCD1070_TNF | 13.1 | 17.9 | 8.2 |
| alpha 4 ng/ml | | | |
| 93105_Dermal Fibroblasts CCD1070_IL-1beta | 27.0 | 19.2 | 0.0 |
| 1 ng/ml | | | |
| 93772_dermal fibroblast_IFN gamma | 0.0 | 0.0 | 5.6 |
| 93771_dermal fibroblast_IL-4 | 2.3 | 2.6 | 7.0 |
| 93260_IBD Colitis 2 | 2.3 | 0.0 | 4.0 |
| 93261_IBD Crohns | 0.0 | 1.1 | 0.7 |
| 735010_Colon_normal | 18.6 | 5.9 | 2.3 |
| 735019_Lung_none | 13.2 | 8.1 | 10.6 |
| 64028-1_Thymus_none | 100.0 | 100.0 | 11.8 |
| 64030-1_Kidney_none | 3.9 | 1.6 | 2.8 |

TABLE 33
Panel 4.1D

| | Relative | | Relative |
|---|---|---|---|
| | Expression (%) | | Expression (%) |
| | 4.1dx4tm6089f_{—} | | 4.1dx4tm6089f_{—} |
| Tissue Name | ag850_b2 | Tissue Name | ag850_b2 |
| 93768_Secondary Th1_anti- | 0.0 | 93100_HUVEC | 0.0 |
| CD28/anti-CD3 | | (Endothelial)_IL-1b | |
| 93769_Secondary Th2_anti- | 0.0 | 93779_HUVEC | 0.8 |
| CD28/anti-CD3 | | (Endothelial)_IFN gamma | |
| 93770_Secondary Tr1_anti- | 0.0 | 93102_HUVEC | 1.0 |
| CD28/anti-CD3 | | (Endothelial)_TNF alpha + IFN | |
| | | gamma | |
| 93573_Secondary Th1_resting | 0.0 | 93101_HUVEC | 0.0 |
| day 4-6 in IL-2 | | (Endothelial)_TNF alpha + IL4 | |
| 93572_Secondary Th2_resting | 0.0 | 93781_HUVEC | 0.0 |
| day 4-6 in IL-2 | | (Endothelial)_IL-11 | |
| 93571_Secondary Tr1_resting | 0.7 | 93583_Lung Microvascular | 0.0 |
| day 4-6 in IL-2 | | Endothelial Cells_none | |
| 93568_primary Th1_anti- | 0.2 | 93584_Lung Microvascular | 0.0 |
| CD28/anti-CD3 | | Endothelial Cells_TNFa (4 ng/ml) | |
| | | and IL1b (1 ng/ml) | |
| 93569_primary Th2_anti- | 0.0 | 92662_Microvascular Dermal | 0.3 |
| CD28/anti-CD3 | | endothelium_none | |
| 93570_primary Tr1_anti- | 0.6 | 92663_Microsvasular Dermal | 0.0 |
| CD28/anti-CD3 | | endothelium_TNFa (4 ng/ml) | |
| | | and IL1b (1 ng/ml) | |
| 93565_primary Th1_resting dy | 0.0 | 93773_Bronchial | 0.9 |
| 4-6 in IL-2 | | epithelium_TNFa (4 ng/ml) and | |
| | | IL1b (1 ng/ml)** | |
| 93566_primary Th2_resting dy | 0.0 | 93347_Small Airway | 1.8 |
| 4-6 in IL-2 | | Epithelium_none | |
| 93567_primary Tr1_resting dy | 0.0 | 93348_Small Airway | 4.0 |
| 4-6 in IL-2 | | Epithelium_TNFa (4 ng/ml) | |
| | | and IL1b (1 ng/ml) | |
| 93351_CD45RA CD4 | 7.4 | 92668_Coronery Artery | 1.8 |
| lymphocyte_anti-CD28/anti- | | SMC_resting | |
| CD3 | | | |
| 93352_CD45RO CD4 | 0.2 | 92669_Coronery Artery | 1.3 |
| lymphocyte_anti-CD28/anti- | | SMC_TNFa (4 ng/ml) and IL1b | |
| CD3 | | (1 ng/ml) | |
| 93251_CD8 Lymphocytes_anti- | 0.0 | 93107_astrocytes_resting | 31.9 |
| CD28/anti-CD3 | | | |
| 93353_chronic CD8 | 0.2 | 93108_astrocytes_TNFa (4 ng/ml) | 33.4 |
| Lymphocytes 2ry_resting dy 4-6 | | and IL1b (1 ng/ml) | |
| in IL-2 | | | |
| 93574_chronic CD8 | 0.3 | 92666_KU-812 | 0.0 |
| Lymphocytes 2ry_activated | | (Basophil)_resting | |
| CD3/CD28 | | | |
| 93354_CD4_none | 0.0 | 92667_KU-812 | 0.0 |
| | | (Basophil)_PMA/ionoycin | |
| 93252_Secondary | 0.1 | 93579_CCD1106 | 0.0 |
| Th1/Th2/Tr1_anti-CD95 CH11 | | (Keratinocytes)_none | |
| 93103_LAK cells_resting | 0.0 | 93580_CCD1106 | 0.0 |
| | | (Keratinocytes)_TNFa and | |
| | | IFNg** | |
| 93788_LAK cells_IL-2 | 0.0 | 93791_Liver Cirrhosis | 1.0 |
| 93787_LAK cells_IL-2 + IL-12 | 0.0 | 93577_NCI-H292 | 2.2 |
| 93789_LAK cells_IL-2 + IFN | 0.0 | 93358_NCI-H292_IL-4 | 1.1 |
| gamma | | | |
| 93790_LAK cells_IL-2 + IL-18 | 0.0 | 93360_NCI-H292_IL-9 | 1.5 |
| 93104_LAK | 0.6 | 93359_NCI-H292_IL-13 | 0.9 |
| cells_PMA/ionomycin and IL- | | | |
| 18 | | | |
| 93578_NK Cells IL-2 resting | 0.0 | 93357_NCI-H292_IFN gamma | 0.3 |
| 93109_Mixed Lymphocyte | 0.0 | 93777_HPAEC_- | 0.0 |
| Reaction_Two Way MLR | | | |
| 93110_Mixed Lymphocyte | 0.0 | 93778_HPAEC_IL-1beta/TNA | 0.0 |
| Reaction_Two Way MLR | | alpha | |
| 93111_Mixed Lymphocyte | 0.2 | 93254_Normal Human Lung | 0.0 |
| Reaction_Two Way MLR | | Fibroblast_none | |
| 93112_Mononuclear Cells | 0.0 | 93253_Normal Human Lung | 0.0 |
| (PBMCs)_resting | | Fibroblast_TNFa (4 ng/ml) and | |
| | | IL-1b (1 ng/ml) | |
| 93113_Mononuclear Cells | 0.0 | 93257_Normal Human Lung | 0.1 |
| (PBMCs)_PWM | | Fibroblast_IL-4 | |
| 93114_Mononuclear Cells | 0.0 | 93256_Normal Human Lung | 0.0 |
| (PBMCs)_PHA-L | | Fibroblast_IL-9 | |
| 93249_Ramos (B cell)_none | 0.0 | 93255_Normal Human Lung | 0.1 |
| | | Fibroblast_IL-13 | |
| 93250_Ramos (B | 0.0 | 93258_Normal Human Lung | 0.8 |
| cell)_ionomycin | | Fibroblast_IFN gamma | |
| 93349_B lymphocytes_PWM | 0.0 | 93106_Dermal Fibroblasts | 14.5 |
| | | CCD1070_resting | |
| 93350_B lymphoytes_CD40L | 0.1 | 93361_Dermal Fibroblasts | 15.2 |
| and IL-4 | | CCD1070_TNF alpha 4 ng/ml | |
| 92665_EOL-1 | 0.0 | 93105_Dermal Fibroblasts | 10.1 |
| (Eosinophil)_dbcAMP | | CCD1070_IL-1beta 1 ng/ml | |
| differentiated | | | |
| 93248_EOL-1 | 0.0 | 93772_dermal fibroblast_IFN | 0.0 |
| (Eosinophil)_dbcAMP/PMAionomycin | | gamma | |
| 93356_Dendritic Cells_none | 0.0 | 93771_dermal fibroblast_IL-4 | 0.0 |
| 93355_Dendritic Cells_LPS | 0.3 | 93892_Dermal fibroblasts_none | 0.0 |
| 100 ng/ml | | | |
| 93775_Dendritic Cells_anti- | 0.0 | 99202_Neutrophils_TNFa + LPS | 0.0 |
| CD40 | | | |
| 93774_Monocytes_resting | 0.0 | 99203_Neutrophils_none | 0.0 |
| 93776_Monocytes_LPS 50 ng/ml | 0.0 | 735010_Colon_normal | 0.6 |
| 93581_Macrophages_resting | 0.0 | 735019_Lung_none | 0.9 |
| 93582_Macrophages_LPS 100 ng/ml | 0.8 | 64028-1_Thymus_none | 6.0 |
| 93098_HUVEC | 0.0 | 64030-1_Kidney_none | 100.0 |
| (Endothelial)_none | | | |
| 93099_HUVEC | 0.0 | | |
| (Endothelial)_starved | | | |

Panel 1 Summary: Ag217 Expression of the NOV4 gene is highest in placenta (CT=21.3). In addition, expression of this gene is also high in testis and uterus tissues. Thus, NOV4 gene expression could be used to distinguish placenta, and to a lesser degree testis and uterus tissue from other tissues. In addition, since these tissues are part of the reproductive system, this gene may play a role in reproduction. Therefore, therapeutic modulation of the NOV4 gene or its product might be of use in the treatment of diseases of reproduction, such as infertility. Furthermore, expression of the NOV4 is much higher in adult kidney (CT=26) than fetal kidney (CT=33) suggesting that this gene may be useful as a marker to distinguish the two.

Panel 1.3D Summary: Ag1469 Expression of the NOV4 gene on this panel is highest in cerebral cortex (CT=27.3). Thus, the expression of this gene might be used to distinguish cerebral cortex from other samples. Among CNS samples, this gene is more moderately expressed in fetal brain, amygdala, hippocampus and thalamus. NOV4 gene expression is also detected in hippocampal and cortical tissue on panel CNS_Neurodegeneration_V1.0, but does not show Alzheimer's disease-specific expression. The NOV4 gene encodes a protein with homology to plexin, a transmembrane cell adhesion molecule that interacts with the semaphorins in axon guidance and CNS development. Semaphorins can act as axon guidance proteins, specifically as chemorepellents that inhibit CNS regenerative capacity. Decreasing levels of this protein may be of use in inducing a compensatory synaptogenic response, as semaphorins are one of the major obstacles to CNS regeneration. Therefore, reducing the level of the NOV4 transcript or its protein product to may be beneficial in the treatment of Alzheimer's disease, Parkinson's disease, Huntington's disease, spinocerebellar ataxia, progressive supranuclear palsy, multiple sclerosis, ALS, head trauma, stroke, or any other disease/condition associated with neuronal loss.

The NOV4 gene is also moderately expressed in adipose and ovary. Interestingly, this gene is more highly expressed in fetal heart (CT=28) when compared to adult heart (CT=33) as well as in fetal skeletal muscle (CT=30) when compared to adult skeletal muscle (CT=37). Thus, the expression of the NOV4 gene might be used to distinguish fetal heart or muscle from adult tissue. Moreover, the therapeutic modulation of this gene, specifically its use in replacement type therapy through the administration of purified protein, might be beneficial in the treatment of diseases involving the degeneration of heart or skeletal muscle, such as the consequence of myocardial infarction or in muscular dystrophy.

General_screeningpanel_v1.4 Summary: Ag217/Ag850 Results from two experiments using different probe/primer sets are in good agreement. Expression of the NOV4 gene on this panel is highest in a sample derived from a brain cancer cell line. In addition, there is substantial expression of this gene in pancreatic, colon, gastric and lung cancer cell lines. Thus, the expression of this gene could be used to distinguish the above mentioned sample types from other tissues. In addition, the therapeutic modulation of the NOV4 gene product, through the use of small molecule drugs or antibodies could be of benefit in the treatment of brain, pancreatic, gastric, colon or lung cancers.

This gene is also expressed at low to moderate levels in a number of other samples on this panel, including kidney, placenta, testis, cerebral cortex and cerebellum.

Panel 2D Summary: Ag1469 Results from three experiments using the same probe/primer set are in good agreement. Expression of the NOV4 gene is highest in an ovarian cancer sample (CT=28). Interestingly, expression of this gene is lower in kidney tumors relative to matched normal kidney margins. This pattern of expression is observed in 6/9 of the normal adjacent kidney/kidney cancer pairs on this panel. Thus, expression of the NOV4 gene could be used as a marker to distinguish normal kidney tissue from kidney tumors and may also have diagnostic benefit. Finally, therapeutic modulation of this gene product might have benefit in the treatment of kidney cancer.

Panel 2.2 Summary: Ag217/Ag850 Results from two experiments using different probe/primer sets are in reasonable agreement. Expression of the NOV4 gene is highest in a sample derived from normal kidney tissue adjacent to a kidney cancer, among the samples on this panel. Furthermore, in the majority of cases this gene is expressed more highly in normal kidney tissues adjacent to kidney cancers. These results are consistent with what is seen in Panel 2D. Thus, expression of the NOV4 gene could be used to distinguish normal kidney tissue from kidney cancer. In addition, therapeutic modulation of the protein encoded by this gene may be of use in the treatment of kidney cancer.

Panel 4D Summary: Ag217/Ag1469 Results from two experiments using different probe/primer sets show some differences. Using the Ag1469 probe/primer set, the NOV4 gene is moderately expressed in the microvascular dermal and lung endothelium (CT 29.2) as well as in primary Tr1 and Th2 T cells. With the Ag217 probe/primer set, the NOV4 gene is expressed at moderate levels in thymus and astrocytes independent of treatment. In addition, lower expression of this gene is seen in dermal fibroblasts. The protein encoded by the NOV4 gene is homologous to murine plexin 2, a transmembrane cell-adhesion molecule. Plexins are receptors for multiple (and perhaps all) classes of semaphorins. Semaphorins influence neural regeneration. Therefore, the expression of this gene in astrocytes suggests that the use of small molecule drugs could be favorable for the treatment of CNS injury. Expression of the NOV4 gene in thymus and some T cells (with Ag1469) suggests a potential role for this plexin in development or differentiation. Therefore, antibodies raised against this protein might be useful as a marker or to modulate T cell differentiation for the treatment of T cell-mediated diseases.

Panel 4.1D Summary: Ag850 The NOV4 gene is expressed in normal kidney, thymus, a dermal fibroblast cell line (CCD1070) and in astrocytes, among the samples on this panel. Expression of this gene is highest in kidney (CT=28.1), consistent with what is observed in Panel 2.2. The NOV4 gene encodes a protein that has homology to retinoic acid-responsive protein, which is known to be expressed at blood organ barriers and may function in transport (ref. 3). Astrocytes contribute to the blood brain barrier (ref. 4), so based on the homology of this gene to Stra6 and its expression in astrocytes, the NOV4 gene product may be important in maintaining the blood brain barrier and perhaps in the transport of small molecules across this barrier. Therefore, regulation of NOV4 gene product with small molecule therapeutics could allow the passage of specific therapeutic molecules into the brain usually blocked by tight junctions. Furthermore, modulation of the function of this protein may also be important in the treatment of autism, since it has been recently shown that autism may be linked to pathology associated with infection and this particular gene is found within a chromosomal locus associated with autism (ref. 5).

References:

1. Murakami Y., Suto F., Shimizu M., Shinoda T., Kameyama T., Fujisawa H. (2001) Differential expression of plexin-A subfamily members in the mouse nervous system. Dev. Dyn. 220:246-258.

Plexins comprise a family of transmembrane proteins (the plexin family) which are expressed in nervous tissues. Some plexins have been shown to interact directly with secreted or transmembrane semaphorins, while plexins belonging to the A subfamily are suggested to make complexes with other membrane proteins, neuropilins, and propagate chemorepulsive signals of secreted semaphorins of class 3 into cells or neurons. Despite that much information has been gathered on the plexin-semaphorin interaction, the role of plexins in the nervous system is not well understood. To gain insight into the functions of plexins in the nervous system, spatial and temporal expression patterns of three members of the plexin-A subfamily (plexin-A1, -A2, and -A3) were analyzed in the developing mouse nervous system by in situ hybridization analysis in combination with immunohistochemistry. The three plexins are differentially expressed in sensory receptors or neurons in a developmentally regulated manner, suggesting that a particular plexin or set of plexins is shared by neuronal elements and functions as the receptor for semaphorins to regulate neuronal development.

PMID: 11241833

2. Ohta K., Mizutani A., Kawakami A., Murakami Y., Kasuya Y., Takagi S., Tanaka H., Fujisawa H. (1995) Plexin: a novel neuronal cell surface molecule that mediates cell adhesion via a homophilic binding mechanism in the presence of calcium ions. Neuron 14:1189-1199.

Plexin (previously referred to as B2) is a neuronal cell surface molecule that has been identified in Xenopus. cDNA cloning reveals that plexin has no homology to known neuronal cell surface molecules but possesses, in its extracellular segment, three internal repeats of cysteine clusters that are homologous to the cysteine-rich domain of the c-met proto-oncogene protein product. The exogenous plexin proteins expressed on the surfaces of L cells by cDNA transfection mediate cell adhesion via a homophilic binding mechanism, under the presence of calcium ions. Plexin is expressed in the receptors and neurons of particular sensory systems. These findings indicate that plexin is a novel calcium-dependent cell adhesion molecule and suggest its involvement in specific neuronal cell interaction and/or contact.

3. Bouillet P., Sapin V, Chazaud C., Messaddeq N., Decimo D., Dolle P., Chambon P. (1997) Developmental expression pattern of Stra6, a retinoic acid-responsive gene encoding a new type of membrane protein. Mech. Dev. 63: 173-186.

Retinoic acid plays important roles in development, growth and differentiation by regulating the expression of target genes. A new retinoic acid-inducible gene, Stra6, has been identified in P19 embryonal carcinoma cells using a subtractive hybridization cDNA cloning technique. Stra6 codes for a very hydrophobic membrane protein of a new type, which does not display similarities with previously characterized integral membrane proteins. Stra6, which exhibits a specific pattern of expression during development and in the adult, is strongly expressed at the level of blood-organ barriers. Interestingly, in testis Sertoli cells, Stra6 has a spermatogenic cycle-dependent expression which is lost in testes of RAR alpha null mutants where Stra6 is expressed in all tubules. The Stra6 protein may be a component of an as yet unidentified transport machinery.

4. Pardridge W. M. (1999) Blood-brain barrier biology and methodology. J. Neurovirol. 5: 556-69.

The blood-brain barrier (BBB) is formed by epithelial-like high resistance tight junctions within the endothelium of capillaries perfusing the vertebrate brain. Because of the presence of the BBB, circulating molecules gain access to brain cells only via one of two processes: (i) lipid-mediated transport of small molecules through the BBB by free diffusion, or (ii) catalyzed transport. The latter includes carrier-mediated transport processes for low molecular weight nutrients and water soluble vitamins or receptor-mediated transport for circulating peptides (e.g., insulin), plasma proteins (e.g., transferrin), or viruses. While BBB permeability, per se, is controlled by the biochemical properties of the plasma membranes of the capillary endothelial cells, overall brain microvascular biology is a function of the paracrine interactions between the capillary endothelium and the other two major cells comprising the microcirculation of brain, i.e., the capillary pericyte, which shares the basement membrane with the endothelial cell, and the astrocyte foot process, which invests 99% of the abluminal surface of the capillary basement membrane in brain. Microvascular functions frequently ascribed to the capillary endothelium are actually executed by either the capillary pericyte or the capillary astrocyte foot process. With respect to BBB methodology, there are a variety of in vivo methods for studying biological transport across this important membrane. The classical physiologic techniques may now be correlated with modern biochemical and molecular biological approaches using freshly isolated animal or human brain capillaries. Isolated brain capillary endothelial cells can also be grown in tissue culture to form an ‘in vitro BBB’ model. However, BBB research cannot be performed using only the in vitro BBB model, but rather it is necessary to correlate observations made with the in vitro BBB model with in vivo studies.

PMID: 10602397

5. Homig M., Weissenbock H., Horscroft N., Lipkin W. I. (1999) An infection-based model of neurodevelopmental damage. Proc. Natl. Acad. Sci. USA 96: 12102-12107.

Perinatal exposure to infectious agents and toxins is linked to the pathogenesis of neuropsychiatric disorders, but the mechanisms by which environmental triggers interact with developing immune and neural elements to create neurodevelopmental disturbances are poorly understood. Homig et al. describe a model for investigating disorders of central nervous system development based on neonatal rat infection with Boma disease virus, a neurotropic noncytolytic RNA virus. Infection results in abnormal righting reflexes, hyperactivity, inhibition of open-field exploration, and stereotypic behaviors. Architecture is markedly disrupted in hippocampus and cerebellum, with reduction in granule and Purkinje cell numbers. Neurons are lost predominantly by apoptosis, as supported by increased mRNA levels for pro-apoptotic products (Fas, caspase-1), decreased mRNA levels for the anti-apoptotic bcl-x, and in situ labeling of fragmented DNA. Although inflammatory infiltrates are observed transiently in frontal cortex, glial activation (microgliosis>astrocytosis) is prominent throughout the brain and persists for several weeks in concert with increased levels of proinflammatory cytokine mRNAs (interleukins 1alpha, 1beta, and 6 and tumor necrosis factor alpha) and progressive hippocampal and cerebellar damage. The resemblance of these functional and neuropathologic abnormalities to human neurodevelopmental disorders suggests the utility of this model for defining cellular, biochemical, histologic, and functional outcomes of interactions of environmental influences with the developing central nervous system.

NOV5

Expression of gene NOV5 was assessed using the primer-probe set Ag2976, described in Table 34. Results from RTQ-PCR runs are shown in Tables 35, 36, 37, 38, and 39.

TABLE 34
Probe Name Ag2976

| | | | | | |
|---|---|---|---|---|---|
| | | | | Start | SEQ |
| Primers | Sequences | TM | Length | Position | ID NO: |

| | | | | | |
|---|---|---|---|---|---|
| Forward | 5′-ACCCCAAATGGATTCCATTA-3′ | 59 | 20 | 1428 | 109 |
| Probe | FAM-5′-CCCTCATGGATCTGCATAACCACACA-3′-TAMRA | 69.9 | 26 | 1465 | 110 |
| Reverse | 5′-CTTGTGTGTGCATGCTTGTC-3′ | 58.9 | 20 | 1516 | 111 |

TABLE 35
Panel 1.3D

| | Relative Expression (%) | |
|---|---|---|
| | 1.3dtm3946f_{—} | 1.3dx4tm5499f_{—} |
| Tissue Name | ag2976 | ag2976_b2 |
| Liver adenocarcinoma | 0.0 | 0.0 |
| Pancreas | 0.0 | 0.0 |
| Pancreatic ca. CAPAN 2 | 0.0 | 0.0 |
| Adrenal gland | 0.0 | 0.7 |
| Thyroid | 0.0 | 0.0 |
| Salivary gland | 0.0 | 0.0 |
| Pituitary gland | 0.4 | 0.0 |
| Brain (fetal) | 0.7 | 1.0 |
| Brain (whole) | 1.1 | 6.5 |
| Brain (amygdala) | 6.9 | 4.4 |
| Brain (cerebellum) | 0.0 | 0.9 |
| Brain (hippocampus) | 18.9 | 3.3 |
| Brain (substantia nigra) | 1.9 | 7.5 |
| Brain (thalamus) | 2.0 | 5.5 |
| Cerebral Cortex | 5.0 | 5.3 |
| Spinal cord | 0.2 | 0.3 |
| CNS ca. (glio/astro) U87-MG | 1.5 | 0.7 |
| CNS ca. (glio/astro) U-118-MG | 0.6 | 0.4 |
| CNS ca. (astro) SW1783 | 0.3 | 0.0 |
| CNS ca.* (neuro; met) SK-N-AS | 0.0 | 0.0 |
| CNS ca. (astro) SF-539 | 0.0 | 0.0 |
| CNS ca. (astro) SNB-75 | 0.0 | 0.0 |
| CNS ca. (glio) SNB-19 | 0.0 | 0.0 |
| CNS ca. (glio) U251 | 0.0 | 0.5 |
| CNS ca. (glio) SF-295 | 0.0 | 0.0 |
| Heart (fetal) | 0.2 | 0.0 |
| Heart | 0.0 | 0.0 |
| Fetal Skeletal | 6.8 | 3.5 |
| Skeletal muscle | 0.1 | 0.8 |
| Bone marrow | 0.0 | 0.0 |
| Thymus | 0.0 | 0.5 |
| Spleen | 0.0 | 1.8 |
| Lymph node | 100.0 | 0.2 |
| Colorectal | 0.8 | 1.3 |
| Stomach | 54.0 | 100.0 |
| Small intestine | 0.4 | 0.5 |
| Colon ca. SW480 | 0.0 | 0.0 |
| Colon ca.* (SW480 met)SW620 | 1.0 | 1.8 |
| Colon ca. HT29 | 0.2 | 0.0 |
| Colon ca. HCT-116 | 0.0 | 0.0 |
| Colon ca. CaCo-2 | 0.0 | 0.0 |
| 83219 CC Well to Mod Diff | 0.6 | 0.0 |
| (ODO3866) | | |
| Colon ca. HCC-2998 | 0.2 | 0.0 |
| Gastric ca.* (liver met) NCI-N87 | 0.4 | 0.3 |
| Bladder | 0.4 | 0.4 |
| Trachea | 0.0 | 0.0 |
| Kidney | 0.0 | 0.0 |
| Kidney (fetal) | 0.0 | 0.0 |
| Renal ca. 786-0 | 0.3 | 0.0 |
| Renal ca. A498 | 0.2 | 0.5 |
| Renal ca. RXF 393 | 0.0 | 0.0 |
| Renal ca. ACHN | 0.0 | 0.0 |
| Renal ca. UO-31 | 0.0 | 0.0 |
| Renal ca. TK-10 | 0.0 | 0.0 |
| Liver | 0.2 | 0.0 |
| Liver (fetal) | 0.0 | 0.0 |
| Liver ca. (hepatoblast) HepG2 | 0.0 | 0.0 |
| Lung | 0.3 | 0.2 |
| Lung (fetal) | 0.6 | 0.0 |
| Lung ca. (small cell) LX-1 | 0.9 | 3.6 |
| Lung ca. (small cell) NCI-H69 | 3.6 | 2.4 |
| Lung ca. (s.cell var.) SHP-77 | 2.9 | 1.4 |
| Lung ca. (large cell)NCI-H460 | 0.0 | 19.9 |
| Lung ca. (non-sm. cell) A549 | 0.0 | 0.0 |
| Lung ca. (non-s.cell) NCI-H23 | 0.6 | 0.2 |
| Lung ca (non-s.cell) HOP-62 | 0.0 | 0.7 |
| Lung ca. (non-s.cl) NCI-H522 | 0.0 | 0.0 |
| Lung ca. (squam.) SW 900 | 0.0 | 0.0 |
| Lung ca. (squam.) NCI-H596 | 0.2 | 0.2 |
| Mammary gland | 0.2 | 0.0 |
| Breast ca.* (pl. effusion) MCF-7 | 0.3 | 0.4 |
| Breast ca.* (pl.ef) MDA-MB-231 | 0.1 | 0.0 |
| Breast ca.* (pl. effusion) T47D | 0.0 | 0.0 |
| Breast ca. BT-549 | 0.2 | 0.2 |
| Breast ca. MDA-N | 0.3 | 0.0 |
| Ovary | 0.1 | 0.0 |
| Ovarian ca. OVCAR-3 | 0.6 | 0.0 |
| Ovarian ca. OVCAR-4 | 0.0 | 0.0 |
| Ovarian ca. OVCAR-5 | 0.0 | 0.0 |
| Ovarian ca. OVCAR-8 | 2.0 | 0.0 |
| Ovarian ca. IGROV-1 | 0.0 | 0.0 |
| Ovarian ca.* (ascites) SK-OV-3 | 0.0 | 0.0 |
| Uterus | 0.0 | 0.0 |
| Placenta | 0.3 | 0.0 |
| Prostate | 0.0 | 0.0 |
| Prostate ca.* (bone met)PC-3 | 0.1 | 0.0 |
| Testis | 8.8 | 13.6 |
| Melanoma Hs688(A).T | 0.0 | 0.0 |
| Melanoma* (met) Hs688(B).T | 0.0 | 0.0 |
| Melanoma UACC-62 | 0.1 | 0.0 |
| Melanoma M14 | 0.2 | 0.0 |
| Melanoma LOX IMVI | 0.0 | 0.0 |
| Melanoma* (met) SK-MEL-5 | 0.2 | 0.5 |
| Adipose | 0.3 | 0.3 |

TABLE 36
Panel 2D

| | Relative | | Relative |
|---|---|---|---|
| | Expression (%) | | Expression (%) |
| | 2dtm3947f_{—} | | 2dtm3947f_{—} |
| Tissue Name | ag2976 | Tissue Name | ag2976 |
| Normal Colon GENPAK | 12.9 | Kidney NAT Clontech 8120608 | 0.0 |
| 061003 | | | |
| 83219 CC Well to Mod Diff | 20.2 | Kidney Cancer Clontech | 0.0 |
| (ODO3866) | | 8120613 | |
| 83220 CC NAT (ODO3866) | 12.9 | Kidney NAT Clontech 8120614 | 0.0 |
| 83221 CC Gr. 2 rectosigmoid | 0.0 | Kidney Cancer Clontech | 0.0 |
| (ODO3868) | | 9010320 | |
| 83222 CC NAT (ODO3868) | 0.0 | Kidney NAT Clontech 9010321 | 0.3 |
| 83235 CC Mod Diff | 0.0 | Normal Uterus GENPAK | 0.0 |
| (ODO3920) | | 061018 | |
| 83236 CC NAT (ODO3920) | 0.0 | Uterus Cancer GENPAK | 5.2 |
| | | 064011 | |
| 83237 CC Gr. 2 ascend colon | 10.6 | Normal Thyroid Clontech A+ | 0.0 |
| (ODO3921) | | 6570-1 | |
| 83238 CC NAT (ODO3921) | 17.0 | Thyroid Cancer GENPAK | 0.0 |
| | | 064010 | |
| 83241 CC from Partial | 0.0 | Thyroid Cancer INVITROGEN | 0.0 |
| Hepatectomy (ODO4309) | | A302152 | |
| 83242 Liver NAT (ODO4309) | 0.7 | Thyroid NAT INVITROGEN | 0.0 |
| | | A302153 | |
| 87472 Colon mets to lung | 0.3 | Normal Breast GENPAK | 0.0 |
| (OD04451-01) | | 061019 | |
| 87473 Lung NAT (OD04451- | 0.0 | 84877 Breast Cancer | 0.0 |
| 02) | | (OD04566) | |
| Normal Prostate Clontech A+ | 0.0 | 85975 Breast Cancer | 0.0 |
| 6546-1 | | (OD04590-01) | |
| 84140 Prostate Cancer | 0.5 | 85976 Breast Cancer Mets | 1.1 |
| (OD04410) | | (OD04590-03) | |
| 84141 Prostate NAT | 1.6 | 87070 Breast Cancer Metastasis | 0.0 |
| (OD04410) | | (OD04655-05) | |
| 87073 Prostate Cancer | 0.8 | GENPAK Breast Cancer | 0.0 |
| (OD04720-01) | | 064006 | |
| 87074 Prostate NAT | 1.4 | Breast Cancer Res. Gen. 1024 | 6.2 |
| (OD04720-02) | | | |
| Normal Lung GENPAK 061010 | 5.2 | Breast Cancer Clontech | 0.9 |
| | | 9100266 | |
| 83239 Lung Met to Muscle | 9.8 | Breast NAT Clontech 9100265 | 0.0 |
| (ODO4286) | | | |
| 83240 Muscle NAT | 0.0 | Breast Cancer INVITROGEN | 1.4 |
| (ODO4286) | | A209073 | |
| 84136 Lung Malignant Cancer | 0.0 | Breast NAT INVITROGEN | 0.2 |
| (OD03126) | | A2090734 | |
| 84137 Lung NAT (OD03126) | 0.0 | Normal Liver GENPAK | 0.0 |
| | | 061009 | |
| 84871 Lung Cancer (OD04404) | 0.3 | Liver Cancer GENPAK 064003 | 5.7 |
| 84872 Lung NAT (OD04404) | 0.5 | Liver Cancer Research Genetics | 0.0 |
| | | RNA 1025 | |
| 84875 Lung Cancer (OD04565) | 0.8 | Liver Cancer Research Genetics | 0.0 |
| | | RNA 1026 | |
| 84876 Lung NAT (OD04565) | 0.0 | Paired Liver Cancer Tissue | 1.3 |
| | | Research Genetics RNA 6004-T | |
| 85950 Lung Cancer (OD04237- | 1.7 | Paired Liver Tissue Research | 2.5 |
| 01) | | Genetics RNA 6004-N | |
| 85970 Lung NAT (OD04237- | 0.7 | Paired Liver Cancer Tissue | 0.0 |
| 02) | | Research Genetics RNA 6005-T | |
| 83255 Ocular Mel Met to Liver | 0.0 | Paired Liver Tissue Research | 3.1 |
| (ODO4310) | | Genetics RNA 6005-N | |
| 83256 Liver NAT (ODO4310) | 0.0 | Normal Bladder GENPAK | 4.8 |
| | | 061001 | |
| 84139 Melanoma Mets to Lung | 0.0 | Bladder Cancer Research | 2.1 |
| (OD04321) | | Genetics RNA 1023 | |
| 84138 Lung NAT (OD04321) | 0.0 | Bladder Cancer INVITROGEN | 15.2 |
| | | A302173 | |
| Normal Kidney GENPAK | 0.8 | 87071 Bladder Cancer | 2.0 |
| 061008 | | (OD04718-01) | |
| 83786 Kidney Ca, Nuclear | 0.7 | 87072 Bladder Normal | 0.0 |
| grade 2 (OD04338) | | Adjacent (OD04718-03) | |
| 83787 Kidney NAT (OD04338) | 0.3 | Normal Ovary Res. Gen. | 4.6 |
| 83788 Kidney Ca Nuclear grade | 0.3 | Ovarian Cancer GENPAK | 0.4 |
| ½ (OD04339) | | 064008 | |
| 83789 Kidney NAT (OD04339) | 2.0 | 87492 Ovary Cancer | 0.9 |
| | | (OD04768-07) | |
| 83790 Kidney Ca, Clear cell | 0.2 | 87493 Ovary NAT (OD04768- | 0.0 |
| type (OD04340) | | 08) | |
| 83791 Kidney NAT (OD04340) | 1.5 | Normal Stomach GENPAK | 100.0 |
| | | 061017 | |
| 83792 Kidney Ca, Nuclear | 0.0 | Gastric Cancer Clontech | 0.0 |
| grade 3 (OD04348) | | 9060358 | |
| 83793 Kidney NAT (OD04348) | 0.9 | NAT Stomach Clontech | 70.2 |
| | | 9060359 | |
| 87474 Kidney Cancer | 0.0 | Gastric Cancer Clontech | 0.0 |
| (OD04622-01) | | 9060395 | |
| 87475 Kidney NAT (OD04622- | 0.0 | NAT Stomach Clontech | 26.4 |
| 03) | | 9060394 | |
| 85973 Kidney Cancer | 0.3 | Gastric Cancer Clontech | 0.0 |
| (OD04450-01) | | 9060397 | |
| 85974 Kidney NAT (OD04450- | 0.1 | NAT Stomach Clontech | 33.0 |
| 03) | | 9060396 | |
| Kidney Cancer Clontech | 0.0 | Gastric Cancer GENPAK | 2.5 |
| 8120607 | | 064005 | |

TABLE 37
Panel 3D

| | Relative | | Relative |
|---|---|---|---|
| | Expression (%) | | Expression (%) |
| Tissue Name | 3dx4tm5126f_ag2976_a1 | Tissue Name | 3dx4tm5126f_ag2976_a1 |
| 94905_Daoy_Medulloblastoma/ | 7.9 | 94954_Ca Ski_Cervical | 0.0 |
| Cerebellum_sscDNA | | epidermoid carcinoma | |
| | | (metastasis)_sscDNA | |
| 94906_TE671_Medulloblastom/ | 0.0 | 94955_ES-2_Ovarian clear cell | 0.0 |
| Cerebellum_sscDNA | | carcinoma_sscDNA | |
| 94907_D283 | 0.0 | 94957_Ramos/6h stim_{—} | 0.0 |
| Med_Medulloblastoma/Cerebellum_{—} | | Stimulated with | |
| sscDNA | | PMA/ionomycin 6h_sscDNA | |
| 94908_PFSK-1_Primitive | 0.0 | 94958_Ramos/14h stim_{—} | 5.1 |
| Neuroectodermal/Cerebellum_sscDNA | | Stimulated with | |
| | | PMA/ionomycin 14h_sscDNA | |
| 94909_XF-498_CNS_sscDNA | 0.0 | 94962_MEG-01_Chronic | 0.0 |
| | | myelogenous leukemia | |
| | | (megokaryoblast)_sscDNA | |
| 94910_SNB- | 0.0 | 94963_Raji_Burkitt's | 0.0 |
| 78_CNS/glioma_sscDNA | | lymphoma_sscDNA | |
| 94911_SF- | 0.0 | 94964_Daudi_Burkitt's | 0.0 |
| 268_CNS/glioblastoma_sscDNA | | lymphoma_sscDNA | |
| 94912_T98G_Glioblastoma_sscDNA | 0.0 | 94965_U266_B-cell | 0.0 |
| | | plasmacytoma/myeloma_sscDNA | |
| 96776_SK-N- | 0.0 | 94968_CA46_Burkitt's | 6.0 |
| SH_Neuroblastoma | | lymphoma_sscDNA | |
| (metastasis)_sscDNA | | | |
| 94913_SF- | 0.0 | 94970_RL_non-Hodgkin's B- | 0.0 |
| 295_CNS/glioblastoma_sscDNA | | cell lymphoma_sscDNA | |
| 94914_Cerebellum_sscDNA | 19.4 | 94972_JM1_pre-B-cell | 0.0 |
| | | lymphoma/leukemia_sscDNA | |
| 96777_Cerebellum_sscDNA | 0.0 | 94973_Jurkat_T cell | 0.0 |
| | | leukemia_sscDNA | |
| 94916_NCI- | 0.0 | 94974_TF- | 0.0 |
| H292_Mucoepidermoid lung | | 1_Erythroleukemia_sscDNA | |
| carcinoma_sscDNA | | | |
| 94917_DMS-114_Small cell | 15.0 | 94975_HUT 78_T-cell | 2.8 |
| lung cancer_sscDNA | | lymphoma_sscDNA | |
| 94918_DMS-79_Small cell | 69.5 | 94977_U937_Histiocytic | 0.0 |
| lung | | lymphoma_sscDNA | |
| cancer/neuroendocrine_sscDNA | | | |
| 94919_NCI-H146_Small cell | 0.0 | 94980_KU-812_Myelogenous | 0.0 |
| lung | | leukemia_sscDNA | |
| cancer/neuroendocrine_sscDNA | | | |
| 94920_NCI-H526_Small cell | 75.8 | 94981_769-P_Clear cell renal | 0.0 |
| lung | | carcinoma_sscDNA | |
| cancer/neuroendocrine_sscDNA | | | |
| 94921_NCI-N417_Small cell | 0.0 | 94983_Caki-2_Clear cell renal | 0.0 |
| lung | | carcinoma_sscDNA | |
| cancer/neuroendocrine_sscDNA | | | |
| 94923_NCI-H82_Small cell | 0.0 | 94984_SW 839_Clear cell renal | 0.0 |
| lung | | carcinoma_sscDNA | |
| cancer/neuroendocrine_sscDNA | | | |
| 94924_NCI-H157_Squamous | 19.4 | 94986_G401_Wilms' | 0.0 |
| cell lung cancer | | tumor_sscDNA | |
| (metastasis)_sscDNA | | | |
| 94925_NCI-H1155_Large cell | 100.0 | 94987_Hs766T_Pancreatic | 4.5 |
| lung | | carcinoma (LN | |
| cancer/neuroendocrine_sscDNA | | metastasis)_sscDNA | |
| 94926_NCI-H1299_Large cell | 5.8 | 94988_CAPAN-1_Pancreatic | 0.0 |
| lung | | adenocarcinoma (liver | |
| cancer/neuroendocrine_sscDNA | | metastasis)_sscDNA | |
| 94927_NCI-H727_Lung | 17.1 | 94989_SU86.86_Pancreatic | 4.5 |
| carcinoid_sscDNA | | carcinoma (liver | |
| | | metastasis)_sscDNA | |
| 94928_NCI-UMC-11_Lung | 0.0 | 94990_BxPC-3_Pancreatic | 0.0 |
| carcinoid_sscDNA | | adenocarcinoma_sscDNA | |
| 94929_LX-1_Small cell lung | 0.0 | 94991_HPAC_Pancreatic | 0.0 |
| cancer_sscDNA | | adenocarcinoma_sscDNA | |
| 94930_Colo-205_Colon | 0.0 | 94992_MIA PaCa-2 Pancreatic | 0.0 |
| cancer_sscDNA | | carcinoma_sscDNA | |
| 94931_KM12_Colon | 0.0 | 94993_CFPAC-1_Pancreatic | 0.0 |
| cancer_sscDNA | | ductal | |
| | | adenocarcinoma_sscDNA | |
| 94932_KM20L2_Colon | 0.0 | 94994_PANC-1_Pancreatic | 5.0 |
| cancer_sscDNA | | epithelioid ductal | |
| | | carcinoma_sscDNA | |
| 94933_NCI-H716_Colon | 0.0 | 94996_T24_Bladder carcinma | 0.0 |
| cancer_sscDNA | | (transitional cell)_sscDNA | |
| 94935_SW-48_Colon | 0.0 | 94997_5637_Bladder | 0.0 |
| adenocarcinoma_sscDNA | | carcinoma_sscDNA | |
| 94936_SW1116_Colon | 0.0 | 94998_HT-1197_Bladder | 32.2 |
| adenocarcinoma_sscDNA | | carcinoma_sscDNA | |
| 94937_LS 174T_Colon | 0.0 | 94999_UM-UC-3_Bladder | 0.0 |
| adenocarcinoma_sscDNA | | carcinma (transitional | |
| | | cell)_sscDNA | |
| 94938_SW-948_Colon | 0.0 | 95000_A204_Rhabdomyosarcoma_{—} | 0.0 |
| adenocarcinoma_sscDNA | | sscDNA | |
| 94939_SW-480_Colon | 19.4 | 95001_HT- | 0.0 |
| adenocarcinoma_sscDNA | | 1080_Fibrosarcoma_sscDNA | |
| 94940_NCI-SNU-5_Gastric | 0.0 | 95002_MG-63_Osteosarcoma | 0.0 |
| carcinoma_sscDNA | | (bone)_sscDNA | |
| 94941_KATO III_Gastric | 0.0 | 95003_SK-LMS- | 0.0 |
| carcinoma_sscDNA | | 1_Leiomyosarcoma | |
| | | (vulva)_sscDNA | |
| 94943_NCI-SNU-16_Gastric | 5.2 | 95004_SJRH30_Rhabdomyosarcoma | 0.0 |
| carcinoma_sscDNA | | (met to bone | |
| | | marrow)_sscDNA | |
| 94944_NCI-SNU-1_Gastric | 0.0 | 95005_A431_Epidermoid | 13.9 |
| carcinoma_sscDNA | | carcinoma_sscDNA | |
| 94946_RF-1_Gastric | 0.0 | 95007_WM2664_{—} | 0.0 |
| adenocarcinoma_sscDNA | | Melanoma_sscDNA | |
| 94947_RF-48_Gastric | 5.1 | 95010_DU 145_Prostate | 0.0 |
| adenocarcinoma_sscDNA | | carcinoma (brain | |
| | | metastasis)_sscDNA | |
| 96778_MKN-45_Gastric | 0.0 | 95012_MDA-MB-468_Breast | 0.0 |
| carcinoma_sscDNA | | adenocarcinoma_sscDNA | |
| 94949_NCI-N87_Gastric | 0.0 | 95013_SCC-4_Squamous cell | 0.0 |
| carcinoma_sscDNA | | carcinoma of tongue_sscDNA | |
| 94951_OVCAR-5_Ovarian | 11.4 | 95014_SCC-9_Squamous cell | 0.0 |
| carcinoma_sscDNA | | carcinoma of tongue_sscDNA | |
| 94952_RL95-2_Uterine | 0.0 | 95015_SCC-15_Squamous cell | 0.0 |
| carcinoma_sscDNA | | carcinoma of tongue_sscDNA | |
| 94953_HelaS3_Cervical | 0.0 | 95017_CAL 27_Squamous cell | 0.0 |
| adenocarcinoma_sscDNA | | carcinoma of tongue_sscDNA | |

TABLE 38
Panel 4D

| | Relative | | Relative |
|---|---|---|---|
| | Expression | | Expression |
| | (%) | | (%) |
| Tissue Name | 4dtm3948f_ag2976 | Tissue Name | 4dtm3948f_ag2976 |
| 93768_Secondary Th1_anti- | 1.5 | 93100_HUVEC | 0.0 |
| CD28/anti-CD3 | | (Endothelial)_IL-1b | |
| 93769_Secondary Th2_anti- | 0.0 | 93779_HUVEC | 0.0 |
| CD28/anti-CD3 | | (Endothelial)_IFN gamma | |
| 93770_Secondary Tr1_anti- | 0.0 | 93102_HUVEC | 0.0 |
| CD28/anti-CD3 | | (Endothelial)_TNF alpha + IFN | |
| | | gamma | |
| 93573_Secondary Th1_resting | 1.3 | 93101_HUVEC | 0.0 |
| day 4-6 in IL-2 | | (Endothelial)_TNF alpha + IL4 | |
| 93572_Secondary Th2_resting | 1.5 | 93781_HUVEC | 0.0 |
| day 4-6 in IL-2 | | (Endothelial)_IL-11 | |
| 93571_Secondary Tr1_resting | 0.0 | 93583_Lung Microvascular | 0.0 |
| day 4-6 in IL-2 | | Endothelial Cells_none | |
| 93568_primary Th1_anti- | 0.0 | 93584_Lung Microvascular | 0.0 |
| CD28/anti-CD3 | | Endothelial Cells_TNFa (4 ng/ml) | |
| | | and IL1b (1 ng/ml) | |
| 93569_primary Th2_anti- | 0.0 | 92662_Microvascular Dermal | 0.0 |
| CD28/anti-CD3 | | endothelium_none | |
| 93570_primary Tr1_anti- | 0.0 | 92663_Microsvasular Dermal | 0.0 |
| CD28/anti-CD3 | | endothelium_TNFa (4 ng/ml) | |
| | | and IL1b (1 ng/ml) | |
| 93565_primary Th1_resting dy | 0.0 | 93773_Bronchial | 0.0 |
| 4-6 in IL-2 | | epithelium_TNFa (4 ng/ml) and | |
| | | IL1b (1 ng/ml)** | |
| 93566_primary Th2_resting dy | 0.0 | 93347_Small Airway | 0.0 |
| 4-6 in IL-2 | | Epithelium_none | |
| 93567_primary Tr1_resting dy | 0.0 | 93348_Small Airway | 0.0 |
| 4-6 in IL-2 | | Epithelium_TNFa (4 ng/ml) | |
| | | and IL1b (1 ng/ml) | |
| 93351_CD45RA CD4 | 0.0 | 92668_Coronery Artery | 3.0 |
| lymphocyte_anti-CD28/anti- | | SMC_resting | |
| CD3 | | | |
| 93352_CD45RO CD4 | 0.0 | 92669_Coronery Artery | 0.0 |
| lymphocyte_anti-CD28/anti- | | SMC_TNFa (4 ng/ml) and IL1b | |
| CD3 | | (1 ng/ml) | |
| 93251_CD8 Lymphocytes_anti- | 0.0 | 93107_astrocytes_resting | 1.5 |
| CD28/anti-CD3 | | | |
| 93353_chronic CD8 | 0.0 | 93108_astrocytes_TNFa (4 ng/ml) | 0.0 |
| Lymphocytes 2ry_resting dy 4-6 | | and IL1b (1 ng/ml) | |
| in IL-2 | | | |
| 93574_chronic CD8 | 0.0 | 92666_KU-812 | 2.6 |
| Lymphocytes 2ry_activated | | (Basophil)_resting | |
| CD3/CD28 | | | |
| 93354_CD4_none | 3.0 | 92667_KU-812 | 0.0 |
| | | (Basophil)_PMA/ionoycin | |
| 93252_Secondary | 0.0 | 93579_CCD1106 | |
| Th1/Th2/Tr1_anti-CD95 CH11 | | (Keratinocytes)_none | |
| 93103_LAK cells_resting | 0.0 | 93580_CCD1106 | 3.3 |
| | | (Keratinocytes)_TNFa and | |
| | | IFNg** | |
| 93788_LAK cells_IL-2 | 0.0 | 93791_Liver Cirrhosis | 40.6 |
| 93787_LAK cells_IL-2 + IL-12 | 0.0 | 93792_Lupus Kidney | 1.0 |
| 93789_LAK cells_IL-2 + IFN | 0.0 | 93577_NCI-H292 | 0.2 |
| gamma | | | |
| 93790_LAK cells_IL-2 + IL-18 | 0.0 | 93358_NCI-H292_IL-4 | 0.0 |
| 93104_LAK | 2.8 | 93360_NCI-H292_IL-9 | 2.6 |
| cells_PMA/ionomycin and IL- | | | |
| 18 | | | |
| 93578_NK Cells IL-2_resting | 0.0 | 93359_NCI-H292_IL-13 | 0.0 |
| 93109_Mixed Lymphocyte | 0.0 | 93357_NCI-H292_IFN gamma | 0.2 |
| Reaction_Two Way MLR | | | |
| 93110_Mixed Lymphocyte | 0.0 | 93777_HPAEC_- | 1.7 |
| Reaction_Two Way MLR | | | |
| 93111_Mixed Lymphocyte | 0.0 | 93778_HPAEC_IL-1beta/TNA | 0.0 |
| Reaction_Two Way MLR | | alpha | |
| 93112_Mononuclear Cells | 0.0 | 93254_Normal Human Lung | 1.3 |
| (PBMCs)_resting | | Fibroblast_none | |
| 93113_Mononuclear Cells | 1.6 | 93253_Normal Human Lung | 0.0 |
| (PBMCs)_PWM | | Fibroblast_TNFa (4 ng/ml) and | |
| | | IL-1b (1 ng/ml) | |
| 93114_Mononuclear Cells | 0.0 | 93257_Normal Human Lung | 1.7 |
| (PBMCs)_PHA-L | | Fibroblast_IL-4 | |
| 93249_Ramos (B cell)_none | 6.2 | 93256_Normal Human Lung | 0.0 |
| | | Fibroblast_IL-9 | |
| 93250_Ramos (B | 10.7 | 93255_Normal Human Lung | 2.8 |
| cell)_ionomycin | | Fibroblast_IL-13 | |
| 93349_B lymphocytes_PWM | 0.0 | 93258_Normal Human Lung | 0.0 |
| | | Fibroblast_IFN gamma | |
| 93350_B lymphoytes_CD40L | 0.0 | 93106_Dermal Fibroblasts | 0.0 |
| and IL-4 | | CCD1070_resting | |
| 92665_EOL-1 | 0.0 | 93361_Dermal Fibroblasts | 0.0 |
| (Eosinophil)_dbcAMP | | CCD1070_TNF alpha 4 ng/ml | |
| differentiated | | | |
| 93248_EOL-1 | 0.0 | 93105_Dermal Fibroblasts | 0.0 |
| (Eosinophil)_dbcAMP/PMAionomycin | | CCD1070_IL-1beta 1 ng/ml | |
| 93356_Dendritic Cells_none | 0.0 | 93772_dermal fibroblast_IFN | 1.4 |
| | | gamma | |
| 93355_Dendritic Cells_LPS | 5.0 | 93771_dermal fibroblast_IL-4 | 12.5 |
| 100 ng/ml | | | |
| 93775_Dendritic Cells_anti- | 1.1 | 93260_IBD Colitis 2 | 4.9 |
| CD40 | | | |
| 93774_Monocytes_resting | 0.6 | 93261_IBD Crohns | 1.6 |
| 93776_Monocytes_LPS 50 ng/ml | 2.6 | 735010_Colon_normal | 100.0 |
| 93581_Macrophages_resting | 0.4 | 735019_Lung_none | 40.6 |
| 93582_Macrophages_LPS 100 ng/ml | 0.0 | 64028-1_Thymus_none | 2.1 |
| 93098_HUVEC | 1.5 | 64030-1_Kidney_none | 1.6 |
| (Endothelial)_none | | | |
| 93099_HUVEC | 0.0 | | |
| (Endothelial)_starved | | | |

TABLE 39
Panel CNSD.01

| | Relative | | Relative |
|---|---|---|---|
| | Expression (%) | | Expression (%) |
| Tissue Name | cns1x4tm6194f_ag2976_a2 | Tissue Name | cns1x4tm6194f_ag2976_a2 |
| 102633_BA4 Control | 12.0 | 102605_BA17 PSP | 0.0 |
| 102641_BA4 Control2 | 51.2 | 102612_BA17 PSP2 | 5.3 |
| 102625_BA4 Alzheimer's2 | 2.6 | 102637_Sub Nigra Control | 13.9 |
| 102649_BA4 Parkinson's | 23.8 | 102645_Sub Nigra Control2 | 50.3 |
| 102656_BA4 Parkinson's2 | 51.6 | 102629_Sub Nigra | 13.9 |
| | | Alzheimer's2 | |
| 102664_BA4 Huntington's | 25.2 | 102660_Sub Nigra Parkinson's2 | 72.5 |
| 102671_BA4 Huntington's2 | 0.0 | 102667_Sub Nigra | 87.2 |
| | | Huntington's | |
| 102603_BA4 PSP | 0.0 | 102674_Sub Nigra | 47.6 |
| | | Huntington's2 | |
| 102610_BA4 PSP2 | 10.2 | 102614_Sub Nigra PSP2 | 6.1 |
| 102588_BA4 Depression | 6.3 | 102592_Sub Nigra Depression | 1.0 |
| 102596_BA4 Depression2 | 0.0 | 102599_Sub Nigra Depression2 | 0.0 |
| 102634_BA7 Control | 14.2 | 102636_Glob Palladus Control | 6.3 |
| 102642_BA7 Control2 | 55.8 | 102644_Glob Palladus Control2 | 24.9 |
| 102626_BA7 Alzheimer's2 | 9.4 | 102620_Glob Palladus | 15.8 |
| | | Alzheimer's | |
| 102650_BA7 Parkinson's | 3.6 | 102628_Glob Palladus | 1.0 |
| | | Alzheimer's2 | |
| 102657_BA7 Parkinson's2 | 28.3 | 102652_Glob Palladus | 17.6 |
| | | Parkinson's | |
| 102665_BA7 Huntington's | 34.7 | 102659_Glob Palladus | 4.8 |
| | | Parkinson's2 | |
| 102672_BA7 Huntington's2 | 13.6 | 102606_Glob Palladus PSP | 0.0 |
| 102604_BA7 PSP | 6.6 | 102613_Glob Palladus PSP2 | 0.0 |
| 102611_BA7 PSP2 | 0.0 | 102591_Glob Palladus | 5.0 |
| | | Depression | |
| 102589_BA7 Depression | 0.0 | 102638_Temp Pole Control | 9.7 |
| 102632_BA9 Control | 16.1 | 102646_Temp Pole Control2 | 55.7 |
| 102640_BA9 ControL2 | 100.0 | 102622_Temp Pole Alzheimer's | 6.3 |
| 102617_BA9 Alzheimer's | 1.9 | 102630_Temp Pole | 0.0 |
| | | Alzheimer's2 | |
| 102624_BA9 Alzheimer's2 | 11.9 | 102653_Temp Pole Parkinson's | 2.9 |
| 102648_BA9 Parkinson's | 9.3 | 102661_Temp Pole | 17.3 |
| | | Parkinson's2 | |
| 102655_BA9 Parkinson's2 | 29.7 | 102668_Temp Pole | 58.1 |
| | | Huntington's | |
| 102663_BA9 Huntington's | 41.4 | 102607_Temp Pole PSP | 9.8 |
| 102670_BA9 Huntington's2 | 0.0 | 102615_Temp Pole PSP2 | 0.0 |
| 102602_BA9 PSP | 5.3 | 102600_Temp Pole | 12.4 |
| | | Depression2 | |
| 102609_BA9 PSP2 | 0.0 | 102639_Cing Gyr Control | 37.1 |
| 102587_BA9 Depression | 5.9 | 102647_Cing Gyr Control2 | 20.2 |
| 102595_BA9 Depression2 | 0.0 | 102623_Cing Gyr Alzheimer's | 7.2 |
| 102635_BA17 Control | 13.4 | 102631_Cing Gyr Alzheimer's2 | 12.4 |
| 102643_BA17 Control2 | 30.9 | 102654_Cing Gyr Parkinson's | 5.4 |
| 102627_BA17 Alzheimer's2 | 6.2 | 102662_Cing Gyr Parkinson's2 | 32.8 |
| 102651_BA17 Parkinson's | 24.8 | 102669_Cing Gyr Huntington's | 49.7 |
| 102658_BA17 Parkinson's2 | 26.0 | 102676_Cing Gyr | 14.1 |
| | | Huntington's2 | |
| 102666_BA17 Huntington's | 51.1 | 102608_Cing Gyr PSP | 9.0 |
| 102673_BA17 Huntington's2 | 11.8 | 102616_Cing Gyr PSP2 | 5.3 |
| 102590_BA17 Depression | 0.0 | 102594_Cing Gyr Depression | 0.0 |
| 102597_BA17 Depression2 | 4.8 | 102601_Cing Gyr Depression2 | 0.0 |

Panel 1.3D Summary: Ag2976 Results from two experiments using the same probe/primer set show some discrepancies; therefore, only those results that are common between the two experiments will be considered here. The NOV5 gene is expressed at moderate levels in stomach (CT=28-29) and testis (CT=30-31). In addition, low but significant expression of this gene is seen in several parts of the CNS, including amygdala, hippocampus, substantia nigra, thalamus, and cerebral cortex. The NOV5 gene encodes a protein with homology to the dopamine receptor of the D1/D5 class. The dopamine (D2) receptor is the most well-established site of action of all known antipsychotics, suggesting a central role for the dopaminergic system in neuropsychiatric disease. The homology of this receptor with the dopamine D5 receptor makes it an excellent candidate drug target for psychiatric diseases, especially depression, bipolar disorder, schizophrenia, and schizoaffective disorder. In addition, NOV5 gene expression appears to be down regulated in a number of brain cancer cell lines.

Panel 2D Summary: Ag2976 Expression of the NOV5 gene is highest in stomach (CT=30), among the samples on this panel; this result is consistent with what is observed in Panel 1.3D. Interestingly, expression of this gene is lower in 3/3 gastric tumors when compared to the normal margins. Thus, expression of the NOV5 gene could be used to distinguish normal stomach tissue from gastric cancer tissue. In addition, therapeutic modulation of the NOV5 gene product might be of benefit for the treatment of gastric cancers.

Panel 3D Summary: Ag2976 Low but significant expression of the NOV5 gene is limited to three lung cancer cell lines. Thus, expression of this gene might be of use in the distinction of lung cancer cell lines from other cell lines.

Panel 4D Summary: Ag2976 Expression of the NOV5 gene is highest in colon (CT=30.2). Expression of this gene is decreased expression in IBD colitis and IBD Crohn's, suggesting a potential role in these diseases. In addition, the NOV5 gene is expressed at low levels in lung, dermal fibroblasts treated with IL-4, liver cirrhosis and Ramos B cells.

Panel CNSD.01 Summary: Ag2976 The NOV5 gene is expressed at low to moderate levels in the brain, and is present in at least hippocampus, cerebral cortex, subtantia nigra, thalamus, globus palladus and amygdala. In Panel CNS_{—}1, this gene shows decreased expression in several regions of the brain of depressed patients. The dopamine (D2) receptor is the most well-established site of action of all known antipsychotics, suggesting a central role for the dopaminergic system in neuropsychiatric disease. The homology of this receptor with the dopamine D5 receptor, in addition to its downregulation in depression, makes it an excellent candidate drug target for psychiatric diseases, especially depression, bipolar disorder, schizophrenia, and schizoaffective disorder.

NOV7a and NOV7b

Expression of gene NOV7a and NOV7b was assessed using the primer-probe sets Ag760 and Ag1537, described in Tables 40 and 41. Results from RTQ-PCR runs are shown in Tables 42, 43, 44, and 45.

TABLE 40
Probe Name Ag760

| | | | | Start | SEQ |
|---|---|---|---|---|---|
| Primers | Sequences | TM | Length | Position | ID NO: |
| Forward | 5′-CACCATGACAACGACACCTATA-3′ | 58.4 | 22 | 1924 | 112 |
| Probe | TET-5′-ATATGGCACCAACATCACATGCACG-3′-TAMRA | 69.7 | 25 | 1947 | 113 |
| Reverse | 5′-TGGGTAGAAAGTGTGTGTGAAA-3′ | 58.2 | 22 | 1979 | 114 |

TABLE 41
Probe Name Ag1537

| | | | | | |
|---|---|---|---|---|---|
| | | | | Start | SEQ |
| Primers | Sequences | TM | Length | Position | ID NO: |

| | | | | | |
|---|---|---|---|---|---|
| Forward | 5′-AAGGAGCTGGAAGAGAAGAAGA-3′ | 58.9 | 22 | 38 | 115 |
| Probe | FAM-5′-ATCAGAAACTCAGCCCTGGACACCTG-3′-TAMRA | 69.6 | 26 | 92 | 116 |
| Reverse | 5′-GCTGCGACTTGGTCTTGAT-3′ | 59 | 19 | 119 | 117 |

TABLE 42
Panel 1.2

| | Relative | Relative |
|---|---|---|
| | Expression (%) | Expression (%) |
| Tissue Name | 1.2tm880t_ag760 | 1.2tm2211f_ag1537 |
| Endothelial cells | 1.3 | 2.5 |
| Heart (fetal) | 2.3 | 17.6 |
| Pancreas | 74.2 | 35.4 |
| Pancreatic ca. CAPAN 2 | 0.0 | 0.0 |
| Adrenal Gland (new lot*) | 19.1 | 37.4 |
| Thyroid | 100.0 | 14.9 |
| Salavary gland | 15.8 | 34.6 |
| Pituitary gland | 27.4 | 2.1 |
| Brain (fetal) | 0.7 | 0.0 |
| Brain (whole) | 0.5 | 0.2 |
| Brain (amygdala) | 0.3 | 0.3 |
| Brain (cerebellum) | 0.1 | 0.0 |
| Brain (hippocampus) | 0.7 | 0.8 |
| Brain (thalamus) | 0.4 | 0.6 |
| Cerebral Cortex | 0.3 | 0.8 |
| Spinal cord | 0.6 | 0.1 |
| CNS ca. (glio/astro) U87-MG | 0.0 | 0.0 |
| CNS ca. (glio/astro) U-118-MG | 0.0 | 0.0 |
| CNS ca. (astro) SW1783 | 0.0 | 0.0 |
| CNS ca.* (neuro; met) SK-N-AS | 0.0 | 0.0 |
| CNS ca. (astro) SF-539 | 0.0 | 0.0 |
| CNS ca. (astro) SNB-75 | 0.0 | 0.0 |
| CNS ca. (glio) SNB-19 | 0.0 | 0.0 |
| CNS ca. (glio) U251 | 0.2 | 0.1 |
| CNS ca. (glio) SF-295 | 0.0 | 0.1 |
| Heart | 17.0 | 50.3 |
| Skeletal Muscle (new lot*) | 16.0 | 18.2 |
| Bone marrow | 1.4 | 2.7 |
| Thymus | 2.8 | 0.9 |
| Spleen | 30.8 | 29.1 |
| Lymph node | 14.4 | 2.7 |
| Colorectal | 1.1 | 2.3 |
| Stomach | 33.2 | 11.5 |
| Small intestine | 41.5 | 52.5 |
| Colon ca. SW480 | 0.0 | 0.0 |
| Colon ca.* (SW480 met) SW620 | 0.0 | 0.0 |
| Colon ca. HT29 | 0.0 | 0.0 |
| Colon ca. HCT-116 | 0.0 | 0.0 |
| Colon ca. CaCo-2 | 0.0 | 0.0 |
| 83219 CC Well to Mod Diff (ODO3866) | 1.4 | 1.7 |
| Colon ca. HCC-2998 | 0.0 | 0.0 |
| Glastric ca.* (liver met) NCI-N87 | 0.7 | 0.9 |
| Bladder | 13.1 | 52.5 |
| Trachea | 9.6 | 2.1 |
| Kidney | 22.4 | 100.0 |
| Kidney (fetal) | 31.9 | 23.8 |
| Renal ca. 786-0 | 0.0 | 0.0 |
| Renal ca. A498 | 0.1 | 0.0 |
| Renal ca. RXF 393 | 0.0 | 0.0 |
| Renal ca. ACHN | 0.0 | 0.0 |
| Renal ca. UO-31 | 0.0 | 0.0 |
| Renal ca. TK-10 | 0.0 | 0.0 |
| Liver | 1.6 | 2.1 |
| Liver (fetal) | 4.0 | 4.4 |
| Liver ca. (hepatoblast) HepG2 | 0.0 | 0.0 |
| Lung | 4.1 | 1.0 |
| Lung (fetal) | 2.1 | 0.3 |
| Lung ca. (small cell) LX-1 | 0.0 | 0.0 |
| Lung ca. (small cell) NCI-H69 | 0.0 | 0.0 |
| Lung ca. (s.cell var.) SHP-77 | 0.0 | 0.0 |
| Lung ca. (large cell) NCI-H460 | 0.0 | 0.0 |
| Lung ca. (non-sm. cell) A549 | 0.0 | 0.0 |
| Lung ca. (non-s.cell) NCI-H23 | 0.0 | 0.0 |
| Lung ca (non-s.cell) HOP-62 | 0.0 | 0.0 |
| Lung ca. (non-s.cl) NCI-H522 | 0.0 | 0.0 |
| Lung ca. (squam.) SW 900 | 0.0 | 0.0 |
| Lung ca. (squam.) NCI-H596 | 0.0 | 0.0 |
| Mammary gland | 19.3 | 14.8 |
| Breast ca.* (pl. effusion) MCF-7 | 0.0 | 0.0 |
| Breast ca.* (pl.ef) MDA-MB-231 | 0.0 | 0.0 |
| Breast ca.* (pl. effusion) T47D | 0.0 | 0.0 |
| Breast ca. BT-549 | 0.0 | 0.0 |
| Breast ca. MDA-N | 1.2 | 2.2 |
| Ovary | 0.8 | 3.0 |
| Ovarian ca. OVCAR-3 | 0.0 | 0.0 |
| Ovarian ca. OVCAR-4 | 0.0 | 0.0 |
| Ovarian ca. OVCAR-5 | 0.1 | 0.1 |
| Ovarian ca. OVCAR-8 | 0.0 | 0.2 |
| Ovarian ca. IGROV-1 | 0.0 | 0.0 |
| Ovarian ca.* (ascites) SK-OV-3 | 0.0 | 0.0 |
| Uterus | 12.8 | 9.2 |
| Placenta | 7.3 | 3.1 |
| Prostate | 12.3 | 19.5 |
| Prostate ca.* (bone met) PC-3 | 0.0 | 0.0 |
| Testis | 1.4 | 0.2 |
| Melanoma Hs688(A).T | 0.0 | 0.0 |
| Melanoma* (met) Hs688(B).T | 0.0 | 0.0 |
| Melanoma UACC-62 | 0.0 | 0.0 |
| Melanoma M14 | 0.0 | 0.0 |
| Melanoma LOX IMVI | 0.0 | 0.0 |
| Melanoma* (met) SK-MEL-5 | 0.0 | 0.0 |

TABLE 43
Panel 1.3D

| | Relative | | Relative |
|---|---|---|---|
| | Exrpession (%) | | Expression (%) |
| Tissue Name | 1.3dx4tm5483t_ag760_b2 | Tissue Name | 1.3dx4tm5483t_ag760_b2 |
| Liver adenocarcinoma | 0.0 | Kidney (fetal) | 33.4 |
| Pancreas | 43.7 | Renal ca. 786-0 | 0.0 |
| Pancreatic ca. CAPAN 2 | 0.0 | Renal ca. A498 | 0.2 |
| Adrenal gland | 21.5 | Renal ca. RXF 393 | 0.0 |
| Thyroid | 79.7 | Renal ca. ACHN | 0.0 |
| Salivary gland | 13.9 | Renal ca. UO-31 | 0.0 |
| Pituitary gland | 13.4 | Renal ca. TK-10 | 0.0 |
| Brain (fetal) | 0.7 | Liver | 1.9 |
| Brain (whole) | 0.9 | Liver (fetal) | 12.4 |
| Brain (amygdala) | 1.6 | Liver ca. (hepatoblast) HepG2 | 0.0 |
| Brain (cerebellum) | 0.4 | Lung | 15.3 |
| Brain (hippocampus) | 1.8 | Lung (fetal) | 6.1 |
| Brain (substantia nigra) | 2.3 | Lung ca. (small cell) LX-1 | 0.0 |
| Brain (thalamus) | 2.7 | Lung ca. (small cell) NCI-H69 | 0.0 |
| Cerebral Cortex | 0.7 | Lung ca. (s.cell var.) SHP-77 | 0.0 |
| Spinal cord | 1.7 | Lung ca. (large cell) NCI-H460 | 0.4 |
| CNS ca. (glio/astro) U87-MG | 0.0 | Lung ca. (non-sm. cell) A549 | 0.0 |
| CNS ca. (glio/astro) U-118-MG | 0.0 | Lung ca. (non-s.cell) NCI-H23 | 0.0 |
| CNS ca. (astro) SW1783 | 0.0 | Lung ca (non-s.cell) HOP-62 | 0.0 |
| CNS ca.* (neuro; met) SK-N- | 0.0 | Lung ca. (non-s.cl) NCI-H522 | 0.0 |
| AS | | | |
| CNS ca. (astro) SF-539 | 0.0 | Lung ca. (squam.) SW 900 | 0.0 |
| CNS ca. (astro) SNB-75 | 0.0 | Lung ca. (squam.) NCI-H596 | 0.0 |
| CNS ca. (glio) SNB-19 | 0.0 | Mammary gland | 26.7 |
| CNS ca. (glio) U251 | 0.7 | Breast ca.* (pl. effusion) MCF-7 | 0.0 |
| CNS ca. (glio) SF-295 | 0.0 | Breast ca.* (pl.ef) MDA-MB- | 0.0 |
| | | 231 | |
| Heart (fetal) | 6.9 | Breast ca.* (pl. effusion) T47D | 0.0 |
| Heart | 10.9 | Breast ca. BT-549 | 0.0 |
| Fetal Skeletal | 19.4 | Breast ca. MDA-N | 0.2 |
| Skeletal muscle | 9.9 | Ovary | 1.8 |
| Bone marrow | 7.8 | Ovarian ca. OVCAR-3 | 0.0 |
| Thymus | 6.9 | Ovarian ca. OVCAR-4 | 0.0 |
| Spleen | 90.4 | Ovarian ca. OVCAR-5 | 0.0 |
| Lymph node | 73.5 | Ovarian ca. OVCAR-8 | 0.0 |
| Colorectal | 7.9 | Ovarian ca. IGROV-1 | 0.0 |
| Stomach | 65.6 | Ovarian ca.* (ascites) SK-OV-3 | 0.0 |
| Small intestine | 100.0 | Uterus | 87.5 |
| Colon ca. SW480 | 0.0 | Placenta | 6.4 |
| Colon ca.* (SW480 met) SW620 | 0.0 | Prostate | 11.3 |
| Colon ca. HT29 | 0.0 | Prostate ca.* (bone met) PC-3 | 0.0 |
| Colon ca. HCT-116 | 0.0 | Testis | 2.1 |
| Colon ca. CaCo-2 | 0.0 | Melanoma Hs688(A).T | 0.0 |
| 83219 CC Well to Mod Diff | 24.0 | Melanoma* (met) Hs688(B).T | 0.0 |
| (ODO3866) | | | |
| Colon ca. HCC-2998 | 0.0 | Melanoma UACC-62 | 0.0 |
| Gastric ca.* (liver met) NCI- | 1.7 | Melanoma M14 | 0.0 |
| N87 | | | |
| Bladder | 17.0 | Melanoma LOX IMVI | 0.0 |
| Trachea | 26.9 | Melanoma* (met) SK-MEL-5 | 0.0 |
| Kidney | 18.2 | Adipose | 26.6 |

TABLE 44
Panel 2D

| | Relative | | Relative |
|---|---|---|---|
| | Expression (%) | | Expression (%) |
| Tissue Name | 2dtm2348f_ag1537 | Tissue Name | 2dtm2348f_ag1537 |
| Normal Colon GENPAK | 12.3 | Kidney NAT Clontech 8120608 | 23.5 |
| 061003 | | | |
| 83219 CC Well to Mod Diff | 10.7 | Kidney Cancer Clontech | 21.5 |
| (ODO3866) | | 8120613 | |
| 83220 CC NAT (ODO3866) | 12.2 | Kidney NAT Clontech 8120614 | 12.3 |
| 83221 CC Gr.2 rectosigmoid | 3.2 | Kidney Cancer Clontech | 34.4 |
| (ODO3868) | | 9010320 | |
| 83222 CC NAT (ODO3868) | 0.8 | Kidney NAT Clontech 9010321 | 27.7 |
| 83235 CC Mod Diff | 3.4 | Normal Uterus GENPAK | 9.3 |
| (ODO3920) | | 061018 | |
| 83236 CC NAT (ODO3920) | 2.2 | Uterus Cancer GENPAK | 6.4 |
| | | 064011 | |
| 83237 CC Gr.2 ascend colon | 13.4 | Normal Thyroid Clontech A+ | 84.1 |
| (ODO3921) | | 6570-1 | |
| 83238 CC NAT (ODO3921) | 5.8 | Thyroid Cancer GENPAK | 20.6 |
| | | 064010 | |
| 83241 CC from Partial | 9.6 | Thyroid Cancer INVITROGEN | 15.2 |
| Hepatectomy (ODO4309) | | A302152 | |
| 83242 Liver NAT (ODO4309) | 0.6 | Thyroid NAT INVITROGEN | 21.3 |
| | | A302153 | |
| 87472 Colon mets to lung | 5.5 | Normal Breast GENPAK | 22.1 |
| (OD04451-01) | | 061019 | |
| 87473 Lung NAT (OD04451- | 0.8 | 84877 Breast Cancer | 8.4 |
| 02) | | (OD04566) | |
| Normal Prostate Clontech A+ | 14.1 | 85975 Breast Cancer | 21.0 |
| 6546-1 | | (OD04590-01) | |
| 84140 Prostate Cancer | 8.8 | 85976 Breast Cancer Mets | 27.7 |
| (OD04410) | | (OD04590-03) | |
| 84141 Prostate NAT | 6.9 | 87070 Breast Cancer Metastasis | 9.1 |
| (OD04410) | | (OD04655-05) | |
| 87073 Prostate Cancer | 3.1 | GENPAK Breast Cancer | 10.1 |
| (OD0472001) | | 064006 | |
| 87074 Prostate NAT | 10.3 | Breast Cancer Res. Gen. 1024 | 7.1 |
| (OD04720-02) | | | |
| Normal Lung GENPAK 061010 | 11.8 | Breast Cancer Clontech | 10.4 |
| | | 9100266 | |
| 83239 Lung Met to Muscle | 6.4 | Breast NAT Clontech 9100265 | 7.4 |
| (ODO4286) | | | |
| 83240 Muscle NAT | 9.9 | Breast Cancer INVITROGEN | 27.4 |
| (ODO4286) | | A209073 | |
| 84136 Lung Malignant Cancer | 19.3 | Breast NAT INVITROGEN | 8.7 |
| (OD03126) | | A2090734 | |
| 84137 Lung NAT (OD03126) | 3.3 | Normal Liver GENPAK | 1.1 |
| | | 061009 | |
| 84871 Lung Cancer (OD04404) | 5.2 | Liver Cancer GENPAK 064003 | 6.5 |
| 84872 Lung NAT (OD04404) | 25.3 | Liver Cancer Research Genetics | 0.7 |
| | | RNA 1025 | |
| 84875 Lung Cancer (OD04565) | 3.4 | Liver Cancer Research Genetics | 8.1 |
| | | RNA 1026 | |
| 84876 Lung NAT (OD04565) | 3.1 | Paired Liver Cancer Tissue | 1.9 |
| | | Research Genetics RNA 6004-T | |
| 85950 Lung Cancer (OD04237- | 11.0 | Paired Liver Tissue Research | 3.6 |
| 01) | | Genetics RNA 6004-N | |
| 85970 Lung NAT (OD04237- | 18.2 | Paired Liver Cancer Tissue | 9.3 |
| 02) | | Research Genetics RNA 6005-T | |
| 83255 Ocular Mel Met to Liver | 0.7 | Paired Liver Tissue Research | 0.6 |
| (ODO4310) | | Genetics RNA 6005-N | |
| 83256 Liver NAT (ODO4310) | 1.7 | Normal Bladder GENPAK | 14.1 |
| | | 061001 | |
| 84139 Melanoma Mets to Lung | 3.9 | Bladder Cancer Research | 4.4 |
| (OD04321) | | Genetics RNA 1023 | |
| 84138 Lung NAT (OD04321) | 3.7 | Bladder Cancer INVITROGEN | 3.6 |
| | | A302173 | |
| Nomal Kidney GENPAK | 40.6 | 87071 Bladder Cancer | 7.4 |
| 061008 | | (OD04718-01) | |
| 83786 Kidney Ca, Nuclear | 5.7 | 87072 Bladder Normal | 15.2 |
| grade 2 (OD04338) | | Adjacent (OD04718-03) | |
| 83787 Kidney NAT (OD04338) | 11.1 | Normal Ovary Res. Gen. | 1.4 |
| 83788 Kidney Ca Nuclear grade | 2.5 | Ovarian Cancer GENPAK | 6.5 |
| ½ (OD04339) | | 064008 | |
| 83789 Kidney NAT (OD04339) | 17.6 | 87492 Ovary Cancer | 1.6 |
| | | (OD04768-07) | |
| 83790 Kidney Ca, Clear cell | 100.0 | 87493 Ovary NAT (OD04768- | 9.2 |
| type (OD04340) | | 08) | |
| 83791 Kidney NAT (OD04340) | 22.7 | Normal Stomach GENPAK | 13.5 |
| | | 061017 | |
| 83792 Kidney Ca, Nuclear | 55.1 | Gastric Cancer Clontech | 2.8 |
| grade 3 (OD04348) | | 9060358 | |
| 83793 Kidney NAT (OD04348) | 19.9 | NAT Stomach Clontech | 12.6 |
| | | 9060359 | |
| 87474 Kidney Cancer | 25.0 | Gastric Cancer Clontech | 20.6 |
| (OD04622-01) | | 9060395 | |
| 87475 Kidney NAT (OD04622- | 7.4 | NAT Stomach Clontech | 7.5 |
| 03) | | 9060394 | |
| 85973 Kidney Cancer | 1.3 | Gastric Cancer Clontech | 10.0 |
| (OD04450-01) | | 9060397 | |
| 85974 Kidney NAT (OD04450- | 9.2 | NAT Stomach Clontech | 3.2 |
| 03) | | 9060396 | |
| Kidney Cancer Clontech | 9.2 | Gastric Cancer GENPAK | 6.7 |
| 8120607 | | 064005 | |

TABLE 45
Panel 4D

| | Relative | | Relative |
|---|---|---|---|
| | Expression (%) | | Expression (%) |
| Tissue Name | 4Dtm2478t_ag760 | Tissue Name | 4Dtm2478t_ag760 |
| 93768_Secondary Th1_anti- | 0.0 | 93100_HUVEC | 3.4 |
| CD28/anti-CD3 | | (Endothelial)_IL-1b | |
| 93769_Secondary Th2_anti- | 0.1 | 93779_HUVEC | 36.6 |
| CD28/anti-CD3 | | (Endothelial)_IFN gamma | |
| 93770_Secondary Tr1_anti- | 0.0 | 93102_HUVEC | 4.0 |
| CD28/anti-CD3 | | (Endothelial)_TNF alpha + IFN | |
| | | gamma | |
| 93573_Secondary Th1_resting | 0.1 | 93101_HUVEC | 3.4 |
| day 4-6 in IL-2 | | (Endothelial)_TNF alpha + IL4 | |
| 93572_Secondary Th2_resting | 0.0 | 93781_HUVEC | 5.5 |
| day 4-6 in IL-2 | | (Endothelial)_IL-11 | |
| 93571_Secondary Tr1_resting | 0.0 | 93583_Lung Microvascular | 47.0 |
| day 4-6 in IL-2 | | Endothelial Cells_none | |
| 93568_primary Th1_anti- | 0.0 | 93584_Lung Microvascular | 22.8 |
| CD28/anti-CD3 | | Endothelial Cells_TNFa (4 ng/ml) | |
| | | and IL1b (1 ng/ml) | |
| 93569_primary Th2_anti- | 0.0 | 92662_Microvascular Dermal | 40.1 |
| CD28/anti-CD3 | | endothelium_none | |
| 93570_primary Tr1_anti- | 0.1 | 92663_Microsvasular Dermal | 17.9 |
| CD28/anti-CD3 | | endothelium_TNFa (4 ng/ml) | |
| | | and IL1b (1 ng/ml) | |
| 93565_primary Th1_resting dy | 0.0 | 93773_Bronchial | 0.0 |
| 4-6 in IL-2 | | epithelium_TNFa (4 ng/ml) and | |
| | | IL1b (1 ng/ml)** | |
| 93566_primary Th2_resting dy | 0.0 | 93347_Small Airway | 0.0 |
| 4-6 in IL-2 | | Epithelium_none | |
| 93567_primary Tr1_resting dy | 0.0 | 93348_Small Airway | 0.0 |
| 4-6 in IL-2 | | Epithelium_TNFa (4 ng/ml) | |
| | | and IL1b (1 ng/ml) | |
| 93351_CD45RA CD4 | 0.6 | 92668_Coronery Artery | 0.0 |
| lymphocyte_anti-CD28/anti- | | SMC_resting | |
| CD3 | | | |
| 93352_CD45RO CD4 | 0.2 | 92669_Coronery Artery | 0.0 |
| lymphocyte_anti-CD28/anti- | | SMC_TNFa (4 ng/ml) and IL1b | |
| CD3 | | (1 ng/ml) | |
| 93251_CD8 Lymphocytes_anti- | 0.0 | 93107_astrocytes_resting | 0.0 |
| CD28/anti-CD3 | | | |
| 93353_chronic CD8 | 0.0 | 93108_astrocytes_TNFa (4 ng/ml) | 0.0 |
| Lymphocytes 2ry_resting dy 4-6 | | and IL1b (1 ng/ml) | |
| in IL-2 | | | |
| 93574_chronic CD8 | 0.0 | 92666_KU-812 | 24.3 |
| Lymphocytes 2ry_activated | | (Basophil)_resting | |
| CD3/CD28 | | | |
| 93354_CD4_none | 0.3 | 92667_KU-812 | 29.7 |
| | | (Basophil)_PMA/ionoycin | |
| 93252_Secondary | 0.0 | 93579_CCD1106 | 0.0 |
| Th1/Th2/Tr1_anti-CD95 CH11 | | (Keratinocytes)_none | |
| 93103_LAK cells_resting | 0.1 | 93580_CCD1106 | 0.0 |
| | | (Keratinocytes)_TNFa and | |
| | | IFNg** | |
| 93788_LAK cells_IL-2 | 0.1 | 93791_Liver Cirrhosis | 19.5 |
| 93787_LAK cells_IL-2 + IL-12 | 0.0 | 93792_Lupus Kidney | 34.4 |
| 93789_LAK cells_IL-2 + IFN | 1.0 | 93577_NCI-H292 | 0.0 |
| gamma | | | |
| 93790_LAK cells_IL-2 + IL-18 | 0.7 | 93358_NCI-H292_IL-4 | 0.0 |
| 93104_LAK | 0.0 | 93360_NCI-H292_IL-9 | 0.0 |
| cells_PMA/ionomycin and IL- | | | |
| 18 | | | |
| 93578_NK Cells IL-2_resting | 0.4 | 93359_NCI-H292_IL-13 | 0.0 |
| 93109_Mixed Lymphocyte | 3.5 | 93357_NCI-H292_IFN gamma | 0.0 |
| Reaction_Two Way MLR | | | |
| 93110_Mixed Lymphocyte | 1.3 | 93777_HPAEC_- | 0.9 |
| Reaction_Two Way MLR | | | |
| 93111_Mixed Lymphocyte | 0.0 | 93778_HPAEC_IL-1beta/TNA | 0.7 |
| Reaction_Two Way MLR | | alpha | |
| 93112_Mononuclear Cells | 0.1 | 93254_Normal Human Lung | 0.0 |
| (PBMCs)_resting | | Fibroblast_none | |
| 93113_Mononuclear Cells | 0.0 | 93253_Normal Human Lung | 0.0 |
| (PBMCs)_PWM | | Fibroblast_TNFa (4 ng/ml) and | |
| | | IL-1b (1 ng/ml) | |
| 93114_Mononuclear Cells | 0.1 | 93257_Normal Human Lung | 0.0 |
| (PBMCs)_PHA-L | | Fibroblast_IL-4 | |
| 93249_Ramos (B cell)_none | 0.0 | 93256_Normal Human Lung | 0.0 |
| | | Fibroblast_IL-9 | |
| 93250_Ramos (B | 0.1 | 93255_Normal Human Lung | 0.0 |
| cell)_ionomycin | | Fibroblast_IL-13 | |
| 93349_B lymphocytes_PWM | 0.0 | 93258_Normal Human Lung | 0.0 |
| | | Fibroblast_IFN gamma | |
| 93350_B lymphoytes_CD40L | 0.3 | 93106_Dermal Fibroblasts | 0.0 |
| and IL-4 | | CCD1070_resting | |
| 92665_EOL-1 | 0.0 | 93361_Dermal Fibroblasts | 0.0 |
| (Eosinophil)_dbcAMP | | CCD1070_TNF alpha 4 ng/ml | |
| differentiated | | | |
| 93248_EOL-1 | 0.0 | 93105_Dermal Fibroblasts | 0.1 |
| (Eosinophil)_dbcAMP/PMAion | | CCD1070_IL-1beta 1 ng/ml | |
| omycin | | | |
| 93356_Dendritic_Cells none | 0.0 | 93772_dermal fibroblast_IFN | 0.0 |
| | | gamma | |
| 93355_Dendritic Cells_LPS | 2.3 | 93771_dermal fibroblast_IL-4 | 0.1 |
| 100 ng/ml | | | |
| 93775_Dendritic Cells_anti- | 0.0 | 93260_IBD Colitis 2 | 1.5 |
| CD40 | | | |
| 93774_Monocytes_resting | 0.8 | 93261_IBD Crohns | 9.0 |
| 93776_Monocytes_LPS 50 ng/ml | 0.0 | 735010_Colon_normal | 40.3 |
| 93581_Macrophages_resting | 0.0 | 735019_Lung_none | 100.0 |
| 93582_Macrophages_LPS 100 ng/ml | 0.6 | 64028-1_Thymus_none | 95.3 |
| 93098_HUVEC | 3.8 | 64030-1_Kidney_none | 59.9 |
| (Endothelial)_none | | | |
| 93099_HUVEC | 16.8 | | |
| (Endothelial)_starved | | | |

Panel 1.2 Summary: Ag760/Ag1537 Results from two experiments using different probe/primer sets show some differences. Using Ag760, expression of the NOV7a gene is high to moderate across many of the normal tissue samples on this panel with highest expression in thyroid (CT=20.1). Using Ag1537, expression of the NOV7a gene is high to moderate across many of the normal tissue samples on this panel with highest expression in kidney (CT=21.6). Although the level of expression in some of the samples varies between the experiments, it is clear that that this gene is exclusively expressed in samples derived from normal tissues and not in cancer cell lines. Thus, expression of this gene could be used to distinguish between normal tissues and cultured cells.

The PV-1-like protein is a plasma membrane protein with an extracellular domain. Expression of this gene is high (CT values less than or equal to 27) in a wide array of metabolic tissues including pancreas, adrenal gland, thyroid, pituitary, adult and fetal heart, skeletal muscle and adult and fetal liver. The extracellular domain of this protein makes it a potential antibody target for the treatment of diseases in any or all of these tissues.

Panel 1.3D Summary: Ag760 Expression of the NOV7a gene is highest in small intestine (CT=26). This gene is exclusively expressed in samples derived from normal tissues and not cancer cell lines, consistent with what is observed in Panel 1.2. Thus, the expression of this gene could be used to distinguish between normal tissues and cultured cells.

Among metabolic tissues expression is high in pancreas, adipose, adrenal gland, thyroid, pituitary gland, heart, skeletal muscle, and liver.

This gene is expressed at low to moderate levels throughout the CNS and is specifically found in amygdala, cerebellum, hippocampus, substantia nigra, thalamus, cerebral cortex and spinal cord.

Panel 2D Summary: Ag1537 Expression of the NOV7a gene is highest in a kidney cancer sample (CT=25). Overall, this gene is widely expressed widely across Panel 2D in both normal and adjacent cancer tissue. However, in a couple of instances, it appears that the NOV7a gene is more highly expressed in kidney cancer tissue than in adjacent normal tissue. Therefore, this gene could be used to distinguish kidney cancers from normal kidney tissue. In addition, therapeutic modulation of this gene, through the use of small molecule drugs or antibodies, might be of benefit in the treatment of kidney cancer.

Panel 4D Summary: Ag760 Expression of the NOV7a gene is highest in lung and thymus (CT=26). High expression of this gene is also seen in normal kidney and colon with more moderate expression in endothelial cells and basophils. Expression of the NOV7a gene in lung and lung microvascular endothelial cells is consistent with the expression pattern observed for the PV-1 protein (1). Antibodies raised against the protein encoded by the NOV7a gene could prevent transendothelial trafficking of inflammatory cells to different tissues sites and therefore have a potential use for treatment of inflammatory diseases including delayed type hypersensitivity, asthma, emphysema, rheumatoid-arthritis and IBD.

References:

1. Stan R. V., Kubitza M., and Palade G. E. (1 999) PV-1 is a component of the fenestral and stomatal diaphragms in fenestrated endothelia. Proc. Natl. Acad. Sci. USA 96:13203-13207.

PV-1 is a novel endothelial protein shown by immunocytochemical tests to be specifically associated with the stomatal diaphragms of caveolae in lung endothelium. Although the highest expression levels of both mRNA and protein are in the lung, PV-1 also has been found to be expressed in other organs. Using a specific antibody to the extracellular domain of PV-1, Stan et al. have extended the survey on the presence of this protein at light and electron microscope level in several rat organs. Stan et al. show that by immunofluorescence the antibody recognizes with high specificity the endothelium of the fenestrated peritubular capillaries of the kidney and those of the intestinal villi, pancreas, and adrenals. By immunolocalization at electron microscope level, the antibody recognizes specifically the diaphragms of the fenestrae and the stomatal diaphragms of caveolae and transendothelial channels in the endothelia of these vascular beds. No signal was detected in the continuous endothelium of the heart, skeletal muscle, intestinal muscularis, or brain capillaries or the nondiaphragmed fenestrated endothelium of kidney glomeruli. Taken together, these findings define the only antigen to be localized thus far in fenestral diaphragms. They also show that the stomatal diaphragms of caveolae and transendothelial channels and the fenestral diaphragms might be biochemically related, in addition to being morphologically similar structures.

PMID: 10557298

NOV8a and NOV8b

Expression of gene NOV8a and its variant was assessed using the primer-probe sets Ag147, Ag718, Ag3681, and Ag4085, described in Tables 46, 47, and 48. Results from RTQ-PCR runs are shown in Tables 49, 50, 51, 52, and 53.

TABLE 46
Probe Name Ag147

| | | | | | |
|---|---|---|---|---|---|
| | | | | Start | SEQ |
| Primers | Sequences | TM | Length | Psoition | ID NO: |

| | | | | |
|---|---|---|---|---|
| Forward | 5′-TGAAGACAGCACCTCCCTATCA-3′ | 22 | 1411 | 118 |
| Probe | FAM-5′-CGGCTCCGTGCTGTCACCCAG-3′-TAMRA | 21 | 1436 | 119 |
| Reverse | 5′-AAGAATCCTCAGCATCGCCATA-3′ | 22 | 1472 | 120 |

TABLE 47
Probe Name Ag718

| | | | | | |
|---|---|---|---|---|---|
| | | | | Start | SEQ |
| Primers | Sequences | TM | Length | Position | ID NO: |

| | | | | | |
|---|---|---|---|---|---|
| Forward | 5′-AGAAGGAATCTCTGGGAAAGCT-3′ | 59.9 | 22 | 949 | 121 |
| Probe | FAM-5′-CCACTGGAGATGCTTGTGTCTCTACCA-3′-TAMRA | 68.6 | 27 | 973 | 122 |
| Reverse | 5′-GACAGAGCACTGGCTAGTTCAC-3′ | 59.2 | 22 | 1003 | 123 |

TABLE 48
uz,9/39 Probe Name Ag3681/Ag4085 (identical sequences)

| | | | | Start | SEQ ID |
|---|---|---|---|---|---|
| Primers | Sequences | TM | Length | Position | NO: |
| Forward | 5′-GAATCATCCACAAGTCATCCAT-3′ | 58.7 | 22 | 5818 | 124 |
| Probe | FAM-5′-CTCACTCCCATCTCATGCCTCCCAG-3′-TAMRA | 71.2 | 25 | 5841 | 125 |
| Reverse | 5′-CATGCTGAATGATCGTGACA-3′ | 59.2 | 20 | 5879 | 126 |

TABLE 49
Panel 1

| | Relative | | Relative |
|---|---|---|---|
| | Expression (%) | | Expression (%) |
| Tissue Name | tm289f | Tissue Name | tm289f |
| Endothelial cells | 0.0 | Renal ca. 786-0 | 1.3 |
| Endothelial cells (treated) | 1.2 | Renal ca. A498 | 0.4 |
| Pancreas | 1.6 | Renal ca. RXF 393 | 0.4 |
| Pancreatic ca. CAPAN 2 | 0.5 | Renal ca. ACHN | 1.1 |
| Adrenal gland | 3.0 | Renal ca. UO-31 | 1.4 |
| Thyroid | 0.4 | Renal ca. TK-10 | 0.8 |
| Salavary gland | 0.9 | Liver | 0.5 |
| Pituitary gland | 2.0 | Liver (fetal) | 0.2 |
| Brain (fetal) | 7.7 | Liver ca. (hepatoblast) HepG2 | 0.2 |
| Brain (whole) | 45.1 | Lung | 7.6 |
| Brain (amygdala) | 8.0 | Lung (fetal) | 3.2 |
| Brain (cerebellum) | 49.0 | Lung ca. (small cell) LX-1 | 0.2 |
| Brain (hippocampus) | 8.4 | Lung ca. (small cell) NCI-H69 | 0.6 |
| Brain (substantia nigra) | 11.7 | Lung ca. (s.cell var.) SHP-77 | 0.0 |
| Brain (thalamus) | 5.3 | Lung ca. (large cell) NCI-H460 | 0.0 |
| Brain (hypothalamus) | 1.5 | Lung ca. (non-sm. cell) A549 | 0.4 |
| Spinal cord | 5.6 | Lung ca. (non-s.cell) NCI-H23 | 0.4 |
| CNS ca. (glio/astro) U87-MG | 0.2 | Lung ca. (non-s.cell) HOP-62 | 2.4 |
| CNS ca. (glio/astro) U-118-MG | 0.0 | Lung ca. (non-s.cl) NCI-H522 | 0.9 |
| CNS ca. (astro) SW1783 | 0.0 | Lung ca. (squam.) SW 900 | 2.4 |
| CNS ca.* (neuro; met) SK-N- | 0.2 | Lung ca. (squam.) NCI-H596 | 0.1 |
| AS | | | |
| CNS ca. (astro) SF-539 | 0.8 | Mammary gland | 15.6 |
| CNS ca. (astro) SNB-75 | 0.8 | Breast ca.* (pl. effusion) MCF-7 | 0.8 |
| CNS ca. (glio) SNB-19 | 8.6 | Breast ca.* (pl.ef) MDA-MB- | 0.4 |
| | | 231 | |
| CNS ca. (glio) U251 | 0.7 | Breast ca.* (pl. effusion) T47D | 10.7 |
| CNS ca. (glio) SF-295 | 3.6 | Breast ca. BT-549 | 0.0 |
| Heart | 11.3 | Breast ca. MDA-N | 0.3 |
| Skeletal muscle | 1.6 | Ovary | 8.1 |
| Bone marrow | 0.3 | Ovarian ca. OVCAR-3 | 0.7 |
| Thymus | 6.4 | Ovarian ca. OVCAR-4 | 4.7 |
| Spleen | 0.5 | Ovarian ca. OVCAR-5 | 1.3 |
| Lymph node | 1.2 | Ovarian ca. OVCAR-8 | 1.3 |
| Colon (ascending) | 1.6 | Ovarian ca. IGROV-1 | 0.5 |
| Stomach | 5.9 | Ovarian ca.* (ascites) SK-OV-3 | 1.3 |
| Small intestine | 1.7 | Uterus | 9.0 |
| Colon ca. SW480 | 2.4 | Placenta | 32.1 |
| Colon ca.* (SW480 met) SW620 | 0.2 | Prostate | 1.9 |
| Colon ca. HT29 | 0.2 | Prostate ca.* (bone met) PC-3 | 0.0 |
| Colon ca. HCT-116 | 0.0 | Testis | 100.0 |
| Colon ca. CaCo-2 | 1.1 | Melanoma Hs688(A).T | 0.2 |
| Colon ca. HCT-15 | 0.7 | Melanoma* (met) Hs688(B).T | 0.2 |
| Colon ca. HCC-2998 | 1.5 | Melanoma UACC-62 | 0.0 |
| Gastric ca.* (liver met) NCI- | 11.4 | Melanoma M14 | 0.1 |
| N87 | | | |
| Bladder | 2.3 | Melanoma LOX IMVI | 0.2 |
| Trachea | 3.2 | Melanoma* (met) SK-MEL-5 | 0.0 |
| Kidney | 6.7 | Melanoma SK-MEL-28 | 0.1 |
| Kidney (fetal) | 6.1 | | |

TABLE 50
Panel 1.2

Relative Expression (%)

| Tissue Name | 1.2tm888f_ag718 | 1.2tm1997f_ag718 | 1.2tm2041f_ag718 |
|---|---|---|---|
| Endothelial cells | 0.6 | 0.4 | 0.2 |
| Heart (fetal) | 7.4 | 10.2 | 8.1 |
| Pancreas | 14.8 | 0.1 | 0.0 |
| Pancreatic ca. CAPAN 2 | 0.0 | 0.0 | 0.0 |
| Adrenal Gland (new lot*) | 5.4 | 4.0 | 4.3 |
| Thyroid | 3.8 | 0.0 | 0.0 |
| Salavary gland | 4.0 | 1.9 | 1.4 |
| Pituitary gland | 19.2 | 0.4 | 0.2 |
| Brain (fetal) | 41.8 | 1.3 | 1.5 |
| Brain (whole) | 37.6 | 1.6 | 3.5 |
| Brain (amygdala) | 16.3 | 7.1 | 5.5 |
| Brain (cerebellum) | 17.3 | 1.8 | 1.6 |
| Brain (hippocampus) | 26.6 | 16.6 | 16.2 |
| Brain (thalamus) | 14.5 | 6.7 | 9.3 |
| Cerebral Cortex | 79.6 | 81.8 | 100.0 |
| Spinal cord | 15.0 | 0.7 | 0.9 |
| CNS ca. (glio/astro) U87-MG | 0.0 | 0.0 | 0.0 |
| CNS ca. (glio/astro) U-118-MG | 0.0 | 0.0 | 0.0 |
| CNS ca. (astro) SW1783 | 0.0 | 0.0 | 0.0 |
| CNS ca.* (neuro; met) SK-N-AS | 2.9 | 0.4 | 0.4 |
| CNS ca. (astro) SF-539 | 2.4 | 1.2 | 1.0 |
| CNS ca. (astro) SNB-75 | 0.7 | 0.4 | 0.2 |
| CNS ca. (glio) SNB-19 | 27.2 | 7.2 | 5.8 |
| CNS ca. (glio) U251 | 4.6 | 0.6 | 0.9 |
| CNS ca. (glio) SF-295 | 7.2 | 7.4 | 5.0 |
| Heart | 73.7 | 100.0 | 79.0 |
| Skeletal Muscle (new lot*) | 36.9 | 17.3 | 15.3 |
| Bone marrow | 0.6 | 0.2 | 0.2 |
| Thymus | 1.7 | 0.1 | 0.0 |
| Spleen | 0.6 | 0.2 | 0.1 |
| Lymph node | 1.9 | 0.0 | 0.0 |
| Colorectal | 0.7 | 0.8 | 0.9 |
| Stomach | 8.4 | 0.3 | 0.6 |
| Small intestine | 4.3 | 1.9 | 1.8 |
| Colon ca. SW480 | 10.2 | 4.3 | 4.1 |
| Colon ca.* (SW480 met) SW620 | 0.3 | 0.0 | 0.0 |
| Colon ca. HT29 | 0.0 | 0.0 | 0.0 |
| Colon ca. HCT-116 | 0.1 | 0.1 | 0.0 |
| Colon ca. CaCo-2 | 1.4 | 0.5 | 0.4 |
| 83219 CC Well to Mod Diff (ODO3866) | 0.3 | 0.3 | 0.3 |
| Colon ca. HCC-2998 | 1.4 | 1.4 | 1.2 |
| Gastric ca.* (liver met) NCI-N87 | 35.4 | 15.8 | 8.7 |
| Bladder | 6.2 | 4.6 | 3.7 |
| Trachea | 4.2 | 0.3 | 0.2 |
| Kidney | 16.7 | 46.7 | 48.0 |
| Kidney (fetal) | 20.2 | 2.8 | 2.9 |
| Renal ca. 786-0 | 1.7 | 0.9 | 0.9 |
| Renal ca. A498 | 0.2 | 0.1 | 0.1 |
| Renal ca. RXF 393 | 0.4 | 0.1 | 0.2 |
| Renal ca. ACHN | 2.9 | 3.1 | 1.9 |
| Renal ca. UO-31 | 1.5 | 1.4 | 1.2 |
| Renal ca. TK-10 | 0.5 | 0.4 | 0.2 |
| Liver | 1.6 | 0.6 | 0.5 |
| Liver (fetal) | 1.1 | 0.4 | 0.5 |
| Liver ca. (hepatoblast) HepG2 | 0.1 | 0.3 | 0.0 |
| Lung | 12.8 | 0.4 | 0.6 |
| Lung (fetal) | 9.6 | 0.5 | 0.6 |
| Lung ca. (small cell) LX-1 | 0.3 | 0.2 | 0.2 |
| Lung ca. (small cell) NCI-H69 | 0.9 | 0.6 | 0.4 |
| Lung ca. (s.cell var.) SHP-77 | 0.3 | 0.0 | 0.0 |
| Lung ca. (large cell) NCI-H460 | 0.2 | 0.2 | 2.7 |
| Lung ca. (non-sm. cell) A549 | 0.3 | 0.2 | 0.2 |
| Lung ca. (non-s.cell) NCI-H23 | 0.6 | 0.7 | 0.4 |
| Lung ca. (non-s.cell) HOP-62 | 5.0 | 4.6 | 0.6 |
| Lung ca. (non-s.cl) NCI-H522 | 1.0 | 1.2 | 0.5 |
| Lung ca. (squam.) SW 900 | 2.4 | 2.6 | 2.0 |
| Lung ca. (squam.) NCI-H596 | 0.3 | 0.1 | 0.0 |
| Mammary gland | 13.4 | 2.1 | 3.0 |
| Breast ca.* (pl. effusion) MCF-7 | 0.2 | 0.0 | 0.0 |
| Breast ca.* (pl.ef) MDA-MB-231 | 2.1 | 0.9 | 0.4 |
| Breast ca.* (pl. effusion) T47D | 14.5 | 9.5 | 8.7 |
| Breast ca. BT-549 | 1.4 | 0.5 | 0.5 |
| Breast ca. MDA-N | 0.2 | 0.1 | 0.0 |
| Ovary | 19.1 | 18.9 | 13.6 |
| Ovarian ca. OVCAR-3 | 6.7 | 3.0 | 2.9 |
| Ovarian ca. OVCAR-4 | 17.8 | 19.6 | 17.2 |
| Ovarian ca. OVCAR-5 | 3.3 | 2.8 | 2.1 |
| Ovarian ca. OVCAR-8 | 1.4 | 1.5 | 1.0 |
| Ovarian ca. IGROV-1 | 1.8 | 1.3 | 0.8 |
| Ovarian ca.* (ascites) SK-OV-3 | 10.3 | 5.1 | 6.4 |
| Uterus | 7.0 | 1.8 | 1.4 |
| Placenta | 100.0 | 3.1 | 3.9 |
| Prostate | 4.0 | 2.8 | 1.4 |
| Prostate ca.* (bone met) PC-3 | 7.7 | 5.4 | 4.4 |
| Testis | 45.7 | 1.5 | 1.3 |
| Melanoma Hs688(A).T | 0.0 | 0.0 | 0.0 |
| Melanoma* (met) Hs688(B).T | 0.1 | 0.0 | 0.0 |
| Melanoma UACC-62 | 0.5 | 0.2 | 0.2 |
| Melanoma M14 | 0.1 | 0.2 | 0.0 |
| Melanoma LOX IMVI | 0.0 | 0.0 | 0.0 |
| Melanoma* (met) SK-MEL-5 | 0.2 | 0.1 | 0.2 |

TABLE 51
Panel 2D

| | Relative Expression (%) | |
|---|---|---|
| Tissue Name | 2dtm2721f_ag718 | 2Dtm2831f_ag718 |
| Normal Colon GENPAK 061003 | 13.6 | 15.9 |
| 83219 CC Well to Mod Diff (ODO3866) | 0.9 | 2.0 |
| 83220 CC NAT (ODO3866) | 1.7 | 1.5 |
| 83221 CC Gr.2 rectosigmoid (ODO3868) | 3.4 | 3.2 |
| 83222 CC NAT (ODO3868) | 2.6 | 2.6 |
| 83235 CC Mod Diff (ODO3920) | 2.4 | 3.0 |
| 83236 CC NAT (ODO3920) | 2.9 | 3.1 |
| 83237 CC Gr.2 ascend colon (ODO3921) | 6.5 | 9.1 |
| 83238 CC NAT (ODO3921) | 1.3 | 1.2 |
| 83241 CC from Partial Hepatectomy (ODO4309) | 1.3 | 0.7 |
| 83242 Liver NAT (ODO4309) | 1.3 | 1.5 |
| 87472 Colon mets to lung (OD04451-01) | 4.5 | 4.9 |
| 87473 Lung NAT (OD04451-02) | 26.8 | 29.7 |
| Normal Prostate Clontech A+ 6546-1 | 5.2 | 5.6 |
| 84140 Prostate Cancer (OD04410) | 12.6 | 11.3 |
| 84141 Prostate NAT (OD04410) | 10.6 | 16.4 |
| 87073 Prostate Cancer (OD04720-01) | 19.6 | 19.3 |
| 87074 Prostate NAT (OD04720-02) | 14.7 | 16.4 |
| Normal Lung GENPAK 061010 | 53.6 | 56.6 |
| 83239 Lung Met to Muscle (ODO4286) | 0.5 | 0.5 |
| 83240 Muscle NAT (ODO4286) | 3.6 | 5.5 |
| 84136 Lung Malignant Cancer (OD03126) | 22.5 | 18.9 |
| 84137 Lung NAT (OD03126) | 73.2 | 79.6 |
| 84871 Lung Cancer (OD04404) | 9.9 | 10.9 |
| 84872 Lung NAT (OD04404) | 29.9 | 31.2 |
| 84875 Lung Cancer (OD04565) | 3.3 | 5.8 |
| 84876 Lung NAT (OD04565) | 50.0 | 47.0 |
| 85950 Lung Cancer (OD04237-01) | 4.6 | 3.0 |
| 85970 Lung NAT (OD04237-02) | 28.3 | 39.2 |
| 83255 Ocular Mel Met to Liver (ODO4310) | 1.6 | 0.6 |
| 83256 Liver NAT (ODO4310) | 0.3 | 0.7 |
| 84139 Melanoma Mets to Lung (OD04321) | 0.6 | 1.3 |
| 84138 Lung NAT (OD04321) | 48.6 | 57.0 |
| Normal Kidney GENPAK 061008 | 59.9 | 63.7 |
| 83786 Kidney Ca, Nuclear grade 2 (OD04338) | 2.4 | 4.2 |
| 83787 Kidney NAT (OD04338) | 21.6 | 23.3 |
| 83788 Kidney Ca Nuclear grade ½ (OD04339) | 2.0 | 2.4 |
| 83789 Kidney NAT (OD04339) | 68.8 | 70.7 |
| 83790 Kidney Ca, Clear cell type (OD04340) | 100.0 | 100.0 |
| 83791 Kidney NAT (OD04340) | 59.0 | 65.1 |
| 83792 Kidney Ca, Nuclear grade 3 (OD04348) | 3.9 | 4.0 |
| 83793 Kidney NAT (OD04348) | 34.9 | 29.9 |
| 87474 Kidney Cancer (OD04622-01) | 6.0 | 12.0 |
| 87475 Kidney NAT (OD04622-03) | 8.1 | 8.7 |
| 85973 Kidney Cancer (OD04450-01) | 0.8 | 1.4 |
| 85974 Kidney NAT (OD04450-03) | 26.2 | 25.5 |
| Kidney Cancer Clontech 8120607 | 1.1 | 0.4 |
| Kidney NAT Clontech 8120608 | 18.6 | 20.6 |
| Kidney Cancer Clontech 8120613 | 3.5 | 3.4 |
| Kidney NAT Clontech 8120614 | 52.8 | 54.0 |
| Kidney Cancer Clontech 9010320 | 10.3 | 9.9 |
| Kidney NAT Clontech 9010321 | 34.2 | 31.0 |
| Normal Uterus GENPAK 061018 | 7.1 | 4.5 |
| Uterus Cancer GENPAK 064011 | 12.9 | 11.8 |
| Normal Thyroid Clontech A+ 6570-1 | 2.5 | 1.8 |
| Thyroid Cancer GENPAK 064010 | 1.5 | 1.8 |
| Thyroid Cancer INVITROGEN A302152 | 3.5 | 3.4 |
| Thyroid NAT INVITROGEN A302153 | 2.3 | 2.7 |
| Normal Breast GENPAK 061019 | 27.5 | 26.6 |
| 84877 Breast Cancer (OD04566) | 6.2 | 11.7 |
| 85975 Breast Cancer (OD04590-01) | 7.5 | 6.2 |
| 85976 Breast Cancer Mets (OD04590-03) | 21.2 | 24.5 |
| 87070 Breast Cancer Metastasis (OD04655-05) | 4.6 | 5.8 |
| GENPAK Breast Cancer 064006 | 15.8 | 18.6 |
| Breast Cancer Res. Gen. 1024 | 29.7 | 26.4 |
| Breast Cancer Clontech 9100266 | 2.4 | 3.6 |
| Breast NAT Clontech 9100265 | 6.5 | 6.2 |
| Breast Cancer INVITROGEN A209073 | 22.5 | 24.7 |
| Breast NAT INVITROGEN A2090734 | 15.1 | 18.4 |
| Normal Liver GENPAK 061009 | 0.7 | 0.7 |
| Liver Cancer GENPAK 064003 | 1.6 | 1.3 |
| Liver Cancer Research Genetics RNA 1025 | 1.6 | 1.9 |
| Liver Cancer Research Genetics RNA 1026 | 1.8 | 1.2 |
| Paired Liver Cancer Tissue Research Genetics RNA 6004-T | 0.8 | 1.6 |
| Paired Liver Tissue Research Genetics RNA 6004-N | 0.8 | 1.4 |
| Paired Liver Cancer Tissue Research Genetics RNA 6005-T | 1.3 | 1.0 |
| Paired Liver Tissue Research Genetics RNA 6005-N | 0.0 | 0.0 |
| Normal Bladder GENPAK 061001 | 7.0 | 3.9 |
| Bladder Cancer Research Genetics RNA 1023 | 1.0 | 1.6 |
| Bladder Cancer INVITROGEN A302173 | 12.9 | 16.8 |
| 87071 Bladder Cancer (OD04718-01) | 16.7 | 21.8 |
| 87072 Bladder Normal Adjacent (OD04718-03) | 4.7 | 4.8 |
| Normal Ovary Res. Gen. | 13.7 | 15.3 |
| Ovarian Cancer GENPAK 064008 | 37.6 | 46.3 |
| 87492 Ovary Cancer (OD04768-07) | 24.5 | 22.8 |
| 87493 Ovary NAT (OD04768-08) | 3.6 | 4.4 |
| Normal Stomach GENPAK 061017 | 14.2 | 16.2 |
| Gastric Cancer Clontech 9060358 | 4.6 | 5.5 |
| NAT Stomach Clontech 9060359 | 2.2 | 3.3 |
| Gastric Cancer Clontech 9060395 | 3.2 | 3.0 |
| NAT Stomach Clontech 9060394 | 2.8 | 2.0 |
| Gastric Cancer Clontech 9060397 | 4.6 | 4.7 |
| NAT Stomach Clontech 9060396 | 1.0 | 2.3 |
| Gastric Cancer GENPAK 064005 | 5.3 | 5.3 |

TABLE 52
Panel 4D/4R

| | Relative Expression(%) | | |
|---|---|---|---|
| | 4Dtm1839f_{—} | 4Dtm1909f_{—} | 4rtm2719f_{—} |
| Tissue Name | ag718 | ag718 | ag718 |
| 93768_Secondary Th1_anti-CD28/anti-CD3 | 0.0 | 0.3 | 0.0 |
| 93769_Secondary Th2_anti-CD28/anti-CD3 | 0.1 | 0.1 | 0.5 |
| 93770_Secondary Tr1_anti-CD28/anti-CD3 | 0.6 | 0.0 | 0.9 |
| 93573_Secondary Th1_resting day 4-6 in IL-2 | 0.0 | 0.2 | 0.3 |
| 93572_Secondary Th2_resting day 4-6 in IL-2 | 0.2 | 0.3 | 0.1 |
| 93571_Secondary Tr1_resting day 4-6 in IL-2 | 0.1 | 0.2 | 0.8 |
| 93568_primary Th1_anti-CD28/anti-CD3 | 0.5 | 0.2 | 0.3 |
| 93569_primary Th2_anti-CD28/anti-CD3 | 0.3 | 0.3 | 0.3 |
| 93570_primary Tr1_anti-CD28/anti-CD3 | 0.6 | 0.5 | 0.0 |
| 93565_primary Th1_resting dy 4-6 in IL-2 | 1.0 | 0.3 | 2.3 |
| 93566_primary Th2_resting dy 4-6 in IL-2 | 0.2 | 0.4 | 0.0 |
| 93567_primary Tr1_resting dy 4-6 in IL-2 | 0.2 | 0.0 | 0.0 |
| 93351_CD45RA CD4 lymphocyte_anti- | 0.2 | 0.4 | 0.4 |
| CD28/anti-CD3 | | | |
| 93352_CD45RO CD4 lymphocyte_anti- | 0.2 | 0.0 | 1.2 |
| CD28/anti-CD3 | | | |
| 93251_CD8 Lymphocytes_anti-CD28/anti- | 0.4 | 0.3 | 0.6 |
| CD3 | | | |
| 93353_chronic CD8 Lymphocytes 2ry_resting | 0.4 | 0.7 | 0.8 |
| dy 4-6 in IL-2 | | | |
| 93574_chronic CD8 Lymphocytes | 0.2 | 0.1 | 0.0 |
| 2ry_activated CD3/CD28 | | | |
| 93354_CD4_none | 0.9 | 0.5 | 0.9 |
| 93252_Secondary Th1/Th2/Tr1_anti-CD95 | 0.4 | 0.3 | 0.0 |
| CH11 | | | |
| 93103_LAK cells_resting | 0.2 | 0.3 | 0.5 |
| 93788_LAK cells_IL-2 | 0.4 | 0.3 | 0.7 |
| 93787_LAK cells_IL-2 + IL-12 | 0.0 | 0.2 | 0.0 |
| 93789_LAK cells_IL-2 + IFN gamma | 0.3 | 0.6 | 1.0 |
| 93790_LAK cells_IL-2 + IL-18 | 0.3 | 0.3 | 0.6 |
| 93104_LAK cells_PMA/ionomycin and IL-18 | 0.2 | 0.1 | 0.0 |
| 93578_NK Cells IL-2_resting | 0.0 | 0.7 | 0.8 |
| 93109_Mixed Lymphocyte Reaction_Two | 0.1 | 0.5 | 0.0 |
| Way MLR | | | |
| 93110_Mixed Lymphocyte Reaction_Two | 0.2 | 0.2 | 0.0 |
| Way MLR | | | |
| 93111_Mixed Lymphocyte Reaction_Two | 0.0 | 0.2 | 0.0 |
| Way MLR | | | |
| 93112_Mononuclear Cells (PBMCs)_resting | 0.2 | 0.2 | 0.0 |
| 93113_Mononuclear Cells (PBMCs)_PWM | 0.9 | 0.9 | 1.5 |
| 93114_Mononuclear Cells (PBMCs)_PHA-L | 0.5 | 0.3 | 0.4 |
| 93249_Ramos (B cell)_none | 0.3 | 0.2 | 0.9 |
| 93250_Ramos (B cell)_ionomycin | 0.3 | 0.0 | 0.8 |
| 93349_B lymphocytes_PWM | 0.5 | 0.3 | 1.5 |
| 93350_B lymphoytes_CD40L and IL-4 | 1.2 | 0.9 | 1.2 |
| 92665_EOL-1 (Eosinophil)_dbcAMP | 0.1 | 0.0 | 0.0 |
| differentiated | | | |
| 93248_EOL-1 | 0.4 | 1.0 | 1.5 |
| (Eosinophil)_dbcAMP/PMAionomycin | | | |
| 93356_Dendritic Cells_none | 0.5 | 0.3 | 0.3 |
| 93355_Dendritic Cells_LPS 100 ng/ml | 0.0 | 0.3 | 0.0 |
| 93775_Dendritic Cells_anti-CD40 | 0.1 | 0.2 | 0.0 |
| 93774_Monocytes_resting | 0.3 | 0.0 | 0.4 |
| 93776_Monocytes_LPS 50 ng/ml | 0.6 | 0.5 | 0.0 |
| 93581_Macrophages_resting | 0.2 | 0.4 | 0.0 |
| 93582_Macrophages_LPS 100 ng/ml | 0.2 | 0.2 | 0.0 |
| 93098_HUVEC (Endothelial)_none | 1.4 | 1.5 | 2.1 |
| 93099_HUVEC (Endothelial)_starved | 5.0 | 3.7 | 10.3 |
| 93100_HUVEC (Endothelial)_IL-1b | 3.9 | 3.4 | 21.6 |
| 93779_HUVEC (Endothelial)_IFN gamma | 0.6 | 0.7 | 1.7 |
| 93102_HUVEC (Endothelial)_TNF alpha + IFN | 4.5 | 5.5 | 8.1 |
| gamma | | | |
| 93101_HUVEC (Endothelial)_TNF alpha + IL4 | 4.4 | 4.4 | 6.4 |
| 93781_HUVEC (Endothelial)_IL-11 | 0.8 | 0.6 | 1.3 |
| 93583_Lung Microvascular Endothelial | 6.2 | 5.4 | 8.8 |
| Cells_none | | | |
| 93584_Lung Microvascular Endothelial | 10.2 | 7.4 | 40.3 |
| Cells_TNFa (4 ng/ml) and IL1b (1 ng/ml) | | | |
| 92662_Microvascular Dermal | 17.0 | 17.1 | 9.0 |
| endothelium_none | | | |
| 92663_Microsvasular Dermal | 27.9 | 22.2 | 53.6 |
| endothelium_TNFa (4 ng/ml) and IL1b (1 ng/ml) | | | |
| 93773_Bronchial epithelium_TNFa (4 ng/ml) | 12.1 | 15.4 | 17.6 |
| and IL1b (1 ng/ml)** | | | |
| 93347_Small Airway Epithelium_none | 2.2 | 3.8 | 6.9 |
| 93348_Small Airway Epithelium_TNFa (4 ng/ml) | 36.9 | 28.9 | 63.7 |
| and IL1b (1 ng/ml) | | | |
| 92668_Coronery Artery SMC_resting | 0.4 | 0.2 | 0.9 |
| 92669_Coronery Artery SMC_TNFa (4 ng/ml) | 0.2 | 0.0 | 0.3 |
| and IL1b (1 ng/ml) | | | |
| 93107_astrocytes_resting | 0.3 | 0.5 | 0.8 |
| 93108_astrocytes_TNFa (4 ng/ml) and IL1b (1 ng/ml) | 4.0 | 2.3 | 6.5 |
| 92666_KU-812 (Basophil)_resting | 0.2 | 0.4 | 0.3 |
| 92667_KU-812 (Basophil)_PMA/ionoycin | 0.2 | 0.3 | 0.0 |
| 93579_CCD1106 (Keratinocytes)_none | 6.2 | 5.7 | 10.4 |
| 93580_CCD1106 (Keratinocytes)_TNFa and | 100.0 | 100.0 | 30.6 |
| IFNg** | | | |
| 93791_Liver Cirrhosis | 1.3 | 0.7 | 6.2 |
| 93792_Lupus Kidney | 14.9 | 11.5 | 25.2 |
| 93577_NCI-H292 | 13.7 | 12.2 | 25.0 |
| 93358_NCI-H292_IL-4 | 24.7 | 23.8 | 100.0 |
| 93360_NCI-H292_IL-9 | 13.8 | 10.7 | 28.9 |
| 93359_NCI-H292_IL-13 | 9.5 | 12.0 | 12.7 |
| 93357_NCI-H292_IFN gamma | 8.5 | 10.0 | 15.7 |
| 93777_HPAEC_- | 0.8 | 1.3 | 2.3 |
| 93778_HPAEC_IL-1beta/TNA alpha | 8.7 | 11.0 | 11.7 |
| 93254_Normal Human Lung Fibroblast_none | 0.3 | 0.2 | 0.0 |
| 93253_Normal Human Lung Fibroblast_TNFa | 0.2 | 0.5 | 0.3 |
| (4 ng/ml) and IL-1b (1 ng/ml) | | | |
| 93257_Normal Human Lung Fibroblast_IL-4 | 0.2 | 0.0 | 1.8 |
| 93256_Normal Human Lung Fibroblast_IL-9 | 0.0 | 0.2 | 0.7 |
| 93255_Normal Human Lung Fibroblast_IL-13 | 0.2 | 0.1 | 0.0 |
| 93258_Normal Human Lung Fibroblast_IFN | 0.4 | 0.0 | 0.5 |
| gamma | | | |
| 93106_Dermal Fibroblasts CCD1070_resting | 0.4 | 0.8 | 0.3 |
| 93361_Dermal Fibroblasts CCD1070_TNF | 0.9 | 0.5 | 0.8 |
| alpha 4 ng/ml | | | |
| 93105_Dermal Fibroblasts CCD1070_IL- | 0.3 | 0.7 | 0.3 |
| 1beta 1 ng/ml | | | |
| 93772_dermal fibroblast_IFN gamma | 0.2 | 0.0 | 0.0 |
| 93771_dermal fibroblast_IL-4 | 0.1 | 0.2 | 0.0 |
| 93260_IBD Colitis 2 | 0.1 | 0.3 | 0.3 |
| 93261_IBD Crohns | 0.3 | 0.5 | 1.7 |
| 735010_Colon_normal | 1.6 | 2.8 | 5.6 |
| 735019_Lung_none | 18.9 | 21.8 | 51.0 |
| 64028-1_Thymus_none | 88.3 | 88.9 | 96.6 |
| 64030-1_Kidney_none | 6.8 | 8.0 | 15.7 |

TABLE 53
Panel 4.1D

| | Relative | Relative |
|---|---|---|
| | Expression(%) | Expression(%) |
| | 4.1dx4tm5977f_{—} | 4.1dtm6217f_{—} |
| Tissue Name | ag3681_b1 | ag4085 |
| 93768_Secondary Th1_anti-CD28/anti-CD3 | 1.9 | 3.0 |
| 93769_Secondary Th2_anti-CD28/anti-CD3 | 4.0 | 2.2 |
| 93770_Secondary Tr1_anti-CD28/anti-CD3 | 3.5 | 2.5 |
| 93573_Secondary Th1_resting day 4-6 in IL-2 | 1.9 | 2.0 |
| 93572_Secondary Th2_resting day 4-6 in IL-2 | 4.1 | 0.0 |
| 93571_Secondary Tr1_resting day 4-6 in IL-2 | 2.5 | 2.2 |
| 93568_primary Th1_anti-CD28/anti-CD3 | 2.0 | 5.1 |
| 93569_primary Th2_anti-CD28/anti-CD3 | 3.5 | 1.8 |
| 93570_primary Tr1_anti-CD28/anti-CD3 | 2.4 | 5.4 |
| 93565_primary Th1_resting dy 4-6 in IL-2 | 0.5 | 1.6 |
| 93566_primary Th2_resting dy 4-6 in IL-2 | 1.9 | 0.0 |
| 93567_primary Tr1_resting dy 4-6 in IL-2 | 4.1 | 0.0 |
| 93351_CD45RA CD4 lymphocyte_anti-CD28/anti-CD3 | 3.5 | 1.7 |
| 93352_CD45RO CD4 lymphocyte_anti-CD28/anti-CD3 | 3.3 | 1.9 |
| 93251_CD8 Lymphocytes_anti-CD28/anti-CD3 | 2.6 | 1.4 |
| 93353_chronic CD8 Lymphocytes 2ry_resting dy 4-6 in IL-2 | 3.6 | 0.9 |
| 93574_chronic CD8 Lymphocytes 2ry_activated CD3/CD28 | 1.1 | 1.3 |
| 93354_CD4_none | 2.6 | 2.7 |
| 93252_Secondary Th1/Th2/Tr1_anti-CD95 CH11 | 2.0 | 0.9 |
| 93103_LAK cells_resting | 2.0 | 1.4 |
| 93788_LAK cells_IL-2 | 1.6 | 1.3 |
| 93787_LAK cells_IL-2 + IL-12 | 1.9 | 1.1 |
| 93789_LAK cells_IL-2 + IFN gamma | 2.8 | 0.0 |
| 93790_LAK cells_IL-2 + IL-18 | 2.4 | 0.0 |
| 93104_LAK cells_PMA/ionomycin and IL-18 | 1.7 | 1.1 |
| 93578_NK Cells IL-2_resting | 3.1 | 2.5 |
| 93109_Mixed Lymphocyte Reaction_Two Way MLR | 2.6 | 3.3 |
| 93110_Mixed Lymphocyte Reaction_Two Way MLR | 0.8 | 0.0 |
| 93111_Mixed Lymphocyte Reaction_Two Way MLR | 1.4 | 1.8 |
| 93112_Mononuclear Cells (PBMCs)_resting | 1.8 | 1.2 |
| 93113_Mononuclear Cells (PBMCs)_PWM | 1.2 | 1.4 |
| 93114_Mononuclear Cells (PBMCs)_PHA-L | 1.7 | 0.0 |
| 93249_Ramos (B cell)_none | 1.0 | 0.9 |
| 93250_Ramos (B cell)_ionomycin | 0.0 | 0.0 |
| 93349_B lymphocytes_PWM | 0.3 | 0.8 |
| 93350_B lymphoytes_CD40L and IL-4 | 4.1 | 4.3 |
| 92665_EOL-1 (Eosinophil)_dbcAMP differentiated | 5.6 | 2.6 |
| 93248_EOL-1 (Eosinophil)_dbcAMP/PMAionomycin | 7.1 | 4.3 |
| 93356_Dendritic Cells_none | 1.8 | 3.4 |
| 93355_Dendritic Cells_LPS 100 ng/ml | 0.6 | 0.0 |
| 93775_Dendritic Cells_anti-CD40 | 2.8 | 0.0 |
| 93774_Monocytes_resting | 2.4 | 2.2 |
| 93776_Monocytes_LPS 50 ng/ml | 3.7 | 5.5 |
| 93581_Macrophages_resting | 2.1 | 0.0 |
| 93582_Macrophages_LPS 100 ng/ml | 1.3 | 2.1 |
| 93098_HUVEC (Endothelial)_none | 4.7 | 6.1 |
| 93099_HUVEC (Endothelial)_starved | 6.8 | 5.5 |
| 93100_HUVEC (Endothelial)_IL-1b | 19.5 | 14.9 |
| 93779_HUVEC (Endothelial)_IFN gamma | 4.6 | 8.7 |
| 93102_HUVEC (Endothelial)_TNF alpha + IFN gamma | 24.8 | 32.1 |
| 93101_HUVEC (Endothelial)_TNF alpha + IL4 | 14.4 | 13.3 |
| 93781_HUVEC (Endothelial)_IL-11 | 2.9 | 5.0 |
| 93583_Lung Microvascular Endothelial Cells_none | 27.4 | 29.1 |
| 93584_Lung Microvascular Endothelial Cells_TNFa (4 ng/ml) | 60.1 | 44.4 |
| and IL1b (1 ng/ml) | | |
| 92662_Microvascular Dermal endothelium_none | 20.6 | 18.8 |
| 92663_Microvascular Dermal endothelium_TNFa (4 ng/ml) and | 48.3 | 47.6 |
| IL1b (1 ng/ml) | | |
| 93773_Bronchial epithelium_TNFa (4 ng/ml) and IL1b (1 ng/ml)** | 22.7 | 22.8 |
| 93347_Small Airway Epithelium_none | 6.4 | 6.9 |
| 93348_Small Airway Epithelium_TNFa (4 ng/ml) and IL1b (1 ng/ml) | 36.3 | 27.4 |
| 92668_Coronery Artery SMC_resting | 1.3 | 2.5 |
| 92669_Coronery Artery SMC_TNFa (4 ng/ml) and IL1b (1 ng/ml) | 0.9 | 4.6 |
| 93107_astrocytes_resting | 5.2 | 4.8 |
| 93108_astrocytes_TNFa (4 ng/ml) and IL1b (1 ng/ml) | 7.5 | 13.3 |
| 92666_KU-812 (Basophil)_resting | 0.4 | 2.5 |
| 92667_KU-812 (Basophil)_PMA/ionoycin | 2.3 | 4.7 |
| 93579_CCD1106 (Keratinocytes)_none | 20.2 | 44.8 |
| 93580_CCD1106 (Keratinocytes)_TNFa and IFNg** | 53.3 | 85.9 |
| 93791_Liver Cirrhosis | 6.7 | 2.8 |
| 93577_NCI-H292 | 19.9 | 18.3 |
| 93358_NCI-H292_IL-4 | 30.3 | 39.0 |
| 93360_NCI-H292_IL-9 | 15.2 | 18.8 |
| 93359_NCI-H292_IL-13 | 35.9 | 52.5 |
| 93357_NCI-H292_IFN gamma | 32.2 | 33.7 |
| 93777_HPAEC_- | 6.7 | 12.9 |
| 93778_HPAEC_IL-1beta/TNA alpha | 36.8 | 28.7 |
| 93254_Normal Human Lung Fibroblast_none | 1.2 | 4.0 |
| 93253_Normal Human Lung Fibroblast_TNFa (4 ng/ml) and IL- | 1.1 | 2.4 |
| 1b (1 ng/ml) | | |
| 93257_Normal Human Lung Fibroblast_IL-4 | 2.4 | 0.0 |
| 93256_Normal Human Lung Fibroblast_IL-9 | 1.2 | 1.5 |
| 93255_Normal Human Lung Fibroblast_IL-13 | 0.4 | 2.4 |
| 93258_Normal Human Lung Fibroblast_IFN gamma | 0.7 | 1.5 |
| 93106_Dermal Fibroblasts CCD1070_resting | 3.2 | 5.4 |
| 93361_Dermal Fibroblasts CCD1070_TNF alpha 4 ng/ml | 1.9 | 14.9 |
| 93105_Dermal Fibroblasts CCD1070_IL-1beta 1 ng/ml | 5.7 | 1.4 |
| 93772_dermal fibroblast_IFN gamma | 1.0 | 0.0 |
| 93771_dermal fibroblast_IL-4 | 0.4 | 2.6 |
| 93892_Dermal fibroblasts_none | 0.0 | 1.8 |
| 99202_Neutrophils_TNFa + LPS | 0.0 | 0.9 |
| 99203_Neutrophils_none | 2.9 | 2.4 |
| 735010_Colon_normal | 3.2 | 9.7 |
| 735019_Lung_none | 63.2 | 63.7 |
| 64028-1_Thymus_none | 23.0 | 40.1 |
| 64030-1_Kidney_none | 100.0 | 100.0 |

Panel 1 Summary: Ag147 Expression of the NOV8a gene is highest in testis (CT=25.1). This gene is also highly to moderately expressed throughout the CNS, including in amygdala, cerebellum, hippocampus, substantia nigra, thalamus, hypothalamus and spinal cord, suggesting an important functional role in CNS processes. The NOV8a gene encodes a protein with homology to PAPIN, plakophilin-related armadillo repeat protein-interacting PSD-95/Dlg-A/ZO-1 (PDZ) protein. Ligands of PAPIN are thought to form a complex with p0071/NPRAP/Scatenin and presenilin 1 and may play roles in Notch or Wnt/Wingless pathways. Because presenilin is known to play a role in Alzheimer's disease, inhibitors of interactions between the NOV8A gene product and its biological interactors may be useful in the treatment of Alzheimer's disease or other diseases linked to the Wnt pathway, such as cancer or autism.

Lower levels of expression of the NOV8a gene are also seen in some metabolic tissues including pancreas, adrenal gland, pituitary gland, thyroid, heart, skeletal muscle and liver. Therefore, this gene may play a role in the development of diseases in any or all of these tissues.

Panel 1.2 Summary: Ag718 Results from three experiments using the same probe/primer set show only modest agreement. This discussion pertains to results seen in the majority of the experiments. Expression of the NOV8a gene is highest in heart and cerebral cortex. Thus, this gene could potentially be used to distinguish heart and cerebral cortex from other tissues. This gene is also highly to moderately expressed in other regions of the CNS, including spinal cord, hippocampus, amygdala, cerebellum, and thalamus. Please see Panel 1 summary for description of potential utility of this gene in the CNS.

Panel 2D Summary: Ag718 Results from three experiments using the same probe/primer set are in excellent agreement. Expression of the NOV8a gene in Panel 2D is highest in a sample derived from a kidney cancer. However, the predominant expression pattern in this panel is higher expression in normal tissues when compared to adjacent cancer tissues. This gene is expressed at lower levels 7 of 9 kidney cancers and 5 of 5 lung cancers relative to the normal controls. Thus, expression of the NOV8a gene could be used to distinguish normal kidney or lung tissue from cancerous kidney or lung tissue and may have utility as a diagnostic marker. Finally, therapeutic modulation of this gene product might have benefit in the treatment of lung or kidney cancer.

Panel 4D/4R Summary: Ag718 Results from three experiments using the same probe/primer set are in reasonable agreement. The NOV8a gene is expressed highly in keratinocytes treated with TNFa and IFNg. Significant expression of this gene is also seen in thymus, small airway epithelium and microvascular dermal endothelium treated with TNF-a and IL-1b. Therefore, antibodies against the protein encoded by the NOV8a gene might be useful in down modulating inflammatory responses observed in asthma, emphysema, skin diseases such as psoriasis, and contact hypersensitivity.

Panel 4.1D Summary: Ag3681/Ag4085 The NOV8a gene is expressed highly in keratinocytes treated with TNFa and IFNg, consistent with what is seen in Panels 4D/4R. Significant expression of this gene is also seen in lung microvascular endothelial cells treated with TNFa and IL-1b. Therefore, antibodies against the protein encoded by the NOV8a gene might be useful in down modulating inflammatory responses observed in asthma, emphysema, skin diseases such as psoriasis, and contact hypersensitivity.

References:

1. Deguchi M., Iizuka T., Hata Y., Nishimura W., Hirao K., Yao I., Kawabe H., Takai Y. (2000) PAPIN. A novel multiple PSD-95/Dlg-A/ZO-1 protein interacting with neural plakophilin-related armadillo repeat protein/delta-catenin and p0071. J. Biol. Chem. 275:29875-29880.

A neural plakophilin-related armadillo repeat protein (NPRAP)/delta-catenin interacts with one of Alzheimer disease-related gene products, presenilin 1. Deguchi et al. have previously reported the interaction of NPRAP/delta-catenin with synaptic scaffolding molecule, which is involved in the assembly of synaptic components. NPRAP/delta-catenin also interacts with E-cadherin and beta-catenin and is implicated in the organization of cell-cell junctions. p0071, a ubiquitous isoform of NPRAP/delta-catenin, is localized at desmosomes in HeLa and A431 cells and at adherens junctions in Madin-Darby bovine kidney cells. Deguchi et al. have identified here a novel protein interacting with NPRAP/delta-catenin and p0071 and named this protein plakophilin-related armadillo repeat protein-interacting PSD-95/Dlg-A/ZO-1 (PDZ) protein (PAPIN). PAPIN has six PDZ domains and binds to NPRAP/delta-catenin and p0071 via the second PDZ domain. PAPIN and p0071 are ubiquitously expressed in various tissues and are localized at cell-cell junctions in normal rat kidney cells and bronchial epithelial cells. PAPIN may be a scaffolding protein connecting components of epithelial junctions with p0071.

PMID: 10896674

2. Fraser P. E., Yu .G., Levesque L., Nishimura M., Yang D. S., Mount H. T., Westaway D., St George-Hyslop P. H. (2001) Presenilin function: connections to Alzheimer's disease and signal transduction. Biochem. Soc. Symp. 67:89-100.

Missense mutations in presenilin 1 (PS1) and presenilin 2 (PS2) are associated with early-onset familial Alzheimer's disease which displays an accelerated deposition of amyloid plaques and neurofibrillary tangles. Presenilins are multi-spanning transmembrane proteins which localize primarily to the endoplasmic reticulum and the Golgi compartments. Fraser et al. have previously demonstrated that PS1 exists as a high-molecular-mass complex that is likely to contain several functional ligands. Potential binding proteins were screened by the yeast two-hybrid system using the cytoplasmically orientated PS1 loop domain which was shown to interact strongly with members of the armadillo family of proteins, including beta-catenin, p0071 and a novel neuron-specific plakophilin-related armadillo protein (NPRAP). Armadillo proteins can have dual functions that encompass the stabilization of cellular junctions/synapses and the mediation of signal transduction pathways. These observations suggest that PSI may contribute to both aspects of armadillo-related pathways involving neurite outgrowth and nuclear translocation of beta-catenin upon activation of the wingless (Wnt) pathway. Alzheimer's disease (AD)-related presenilin mutations exhibit a dominant gain of aberrant function resulting in the prevention of beta-catenin translocation following Wnt signalling. These findings indicate a functional role for PSI in signalling and suggest that mistrafficking of selected presenilin ligands may be a potential mechanism in the genesis of AD.

PMID: 11447843

3. Wassink T. H., Piven J., Vieland V. J., Huang J., Swiderski R. E., Pietila J., Braun T., Beck G., Folstein S. E., Haines J. L., Sheffield V. C. (2001) Evidence supporting WNT2 as an autism susceptibility gene. Am. J. Med. Genet. 105:406-413.

Wassink et al. examined WNT2 as a candidate disease gene for autism for the following reasons. First, the WNT family of genes influences the development of numerous organs and systems, including the central nervous system. Second, WNT2 is located in the region of chromosome 7q31-33 linked to autism and is adjacent to a chromosomal breakpoint in an individual with autism. Third, a mouse knockout of Dvl1, a member of a gene family essential for the function of the WNT pathway, exhibits a behavioral phenotype characterized primarily by diminished social interaction. Wassink et al. screened the WNT2 coding sequence for mutations in a large number of autistic probands and found two families containing nonconservative coding sequence variants that segregated with autism in those families. Wassink et al. also identified linkage disequilibrium (LD) between a WNT2 3′UTR SNP and a sample of autism-affected sibling pair (ASP) families and trios. The LD arose almost exclusively from a subgroup of ASP families defined by the presence of severe language abnormalities and was also found to be associated with the evidence for linkage to 7q from our previously published genomewide linkage screen. Furthermore, expression analysis demonstrated WNT2 expression in the human thalamus. Based on these findings, Wassink et al. hypothesize that rare mutations occur in the WNT2 gene that significantly increase susceptibility to autism even when present in single copies, while a more common WNT2 allele (or alleles) not yet identified may exist that contributes to the disorder to a lesser degree.

PMID: 11449391

4. De Ferrari G. V., Inestrosa N. C. (2000) Wnt signaling function in Alzheimer's disease. Brain Res Brain Res Rev 33:1-12.

Alzheimer's disease (AD) is a neurodegenerative disease with progressive dementia accompanied by three main structural changes in the brain: diffuse loss of neurons; intracellular protein deposits termed neurofibrillary tangles (NFT) and extracellular protein deposits termed amyloid or senile plaques, surrounded by dystrophic neurites. Two major hypotheses have been proposed in order to explain the molecular hallmarks of the disease: The ‘amyloid cascade’ hypothesis and the ‘neuronal cytoskeletal degeneration’ hypothesis. While the former is supported by genetic studies of the early-onset familial forms of AD (FAD), the latter revolves around the observation in vivo that cytoskeletal changes—including the abnormal phosphorylation state of the microtubule associated protein tau—may precede the deposition of senile plaques. Recent studies have suggested that the trafficking process of membrane associated proteins is modulated by the FAD-linked presenilin (PS) proteins, and that amyloid beta-peptide deposition may be initiated intracellularly, through the secretory pathway. Current hypotheses concerning presenilin function are based upon its cellular localization and its putative interaction as macromolecular complexes with the cell-adhesion/signaling beta-catenin molecule and the glycogen synthase kinase 3beta (GSK-3beta) enzyme. Developmental studies have shown that PS proteins function as components in the Notch signal transduction cascade and that beta-catenin and GSK-3beta are transducers of the Wnt signaling pathway. Both pathways are thought to have an important role in brain development, and they have been connected through Dishevelled (Dv1) protein, a known transducer of the Wnt pathway. In addition to a review of the current state of research on the subject, DeFerrari et al. present a cell signaling model in which a sustained loss of function of Wnt signaling components would trigger a series of misrecognition events, determining the onset and development of AD.

PMID: 10967351

### Example 3

### SNP Analysis of NOVX Clones

SeqCallingTM Technology: cDNA was derived from various human samples representing multiple tissue types, normal and diseased states, physiological states, and developmental states from different donors. Samples were obtained as whole tissue, cell lines, primary cells or tissue cultured primary cells and cell lines. Cells and cell lines may have been treated with biological or chemical agents that regulate gene expression for example, growth factors, chemokines, steroids. The cDNA thus derived was then sequenced using CuraGen's proprietary SeqCalling technology. Sequence traces were evaluated manually and edited for corrections if appropriate. cDNA sequences from all samples were assembled with themselves and with public ESTs using bioinformatics programs to generate CuraGen's human SeqCalling database of SeqCalling assemblies. Each assembly contains one or more overlapping cDNA sequences derived from one or more human samples. Fragments and ESTs were included as components for an assembly when the extent of identity with another component of the assembly was at least 95% over 50 bp. Each assembly can represent a gene and/or its variants such as splice forms and/or single nucleotide polymorphisms (SNPs) and their combinations.

Variant sequences are included in this application. A variant sequence can include a single nucleotide polymorphism (SNP). A SNP can, in some instances, be referred to as a “cSNP” to denote that the nucleotide sequence containing the SNP originates as a cDNA. A SNP can arise in-several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, however, in the case that a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern for example, alteration in temporal expression, physiological response regulation, cell type expression regulation, intensity of expression, stability of transcribed message.

Method of novel SNP Identification: SNPs are identified by analyzing sequence assemblies using CuraGen's proprietary SNPTool algorithm. SNPTool identifies variation in assemblies with the following criteria: SNPs are not analyzed within 10 base pairs on both ends of an alignment; Window size (number of bases in a view) is 10; The allowed number of mismatches in a window is 2; Minimum SNP base quality (PHRED score) is 23; Minimum number of changes to score an SNP is 2/assembly position. SNPTool analyzes the assembly and displays SNP positions, associated individual variant sequences in the assembly, the depth of the assembly at that given position, the putative assembly allele frequency, and the SNP sequence variation. Sequence traces are then selected and brought into view for manual validation. The consensus assembly sequence is imported into CuraTools along with variant sequence changes to identify potential amino acid changes resulting from the SNP sequence variation. Comprehensive SNP data analysis is then exported into the SNPCalling database.

Method of novel SNP Confirmation: SNPs are confirmed employing a validated method know as Pyrosequencing (Pyrosequencing, Westborough, Mass.). Detailed protocols for Pyrosequencing can be found in: Alderborn et al. Determination of Single Nucleotide Polymorphisms by Real-time Pyrophosphate DNA Sequencing. (2000). Genome Research. 10, Issue 8, August. 1249-1265. In brief, Pyrosequencing is a real time primer extension process of genotyping. This protocol takes double-stranded, biotinylated PCR products from genomic DNA samples and binds them to streptavidin beads. These beads are then denatured producing single stranded bound DNA. SNPs are characterized utilizing a technique based on an indirect bioluminometric assay of pyrophosphate (PPi) that is released from each dNT? upon DNA chain elongation. Following Klenow polymerase-mediated base incorporation, PPi is released and used as a substrate, together with adenosine 5′-phosphosulfate (APS), for ATP sulfurylase, which results in the formation of ATP. Subsequently, the ATP accomplishes the conversion of luciferin to its oxi-derivative by the action of luciferase. The ensuing light output becomes proportional to the number of added bases, up to about four bases. To allow processivity of the method dNTP excess is degraded by apyrase, which is also present in the starting reaction mixture, so that only dNTPs are added to the template during the sequencing. The process has been fully automated and adapted to a 96-well format, which allows rapid screening of large SNP panels. The DNA and protein sequences for the novel single nucleotide polymorphic variants are reported. Variants are reported individually but any combination of all or a select subset of variants are also included. In addition, the positions of the variant bases and the variant amino acid residues are underlined.

### Results

Variants are reported individually but any combination of all or a select subset of variants are also included as contemplated NOVX embodiments of the invention.

NOV1a SNP Data:

NOV1a has two SNP variants, whose variant positions for its nucleotide and amino acid sequences is numbered according to SEQ ID NOs: 1 and 2, respectively. The nucleotide sequence of the NOV1a variant differs as shown in Table 54.

TABLE 54
cSNP and Coding Variants for NOV1a

| NT Position | | | Amino Acid | Amino Acid |
|---|---|---|---|---|
| of cSNP | Wild Type NT | Variant NT | position | Change |
| 994 | C | T | 271 | P->S |
| 1707 | A | G | 508 | No change |

NOV7a SNP Data:

NOV7a has four SNP variants, whose variant positions for its nucleotide and amino acid sequences is numbered according to SEQ ID NOs:X and Y, respectively. The nucleotide sequence of the NOV7a variant differs as shown in Table 55.

TABLE 55
cSNP and Coding Variants for NOV7a

| NT Position | | | Amino Acid | Amino Acid |
|---|---|---|---|---|
| of cSNP | Wild Type NT | Variant NT | position | Change |
| 1023 | C | G | 341 | No change |
| 1247 | C | T | 416 | A->V |

NOV8a SNP Data:

NOV8a has one SNP variant, whose variant position for its nucleotide and amino acid sequences is numbered according to SEQ ID NOs:X and Y, respectively. The nucleotide sequence of the NOV8a variant differs as shown in Table 56.

TABLE 57
cSNP and Coding Variants for NOV8a

| NT Position | | | Amino Acid | Amino Acid |
|---|---|---|---|---|
| of cSNP | Wild Type NT | Variant NT | position | Change |
| 703 | G | A | 201 | No change |
| 4244 | G | A | 1381 | No change |

### Example 4

### SAGE Analysis for NOV1

Serial Analysis of Gene Expression, or SAGE, is an experimental technique designed to gain a quantitative measure of gene expression. The SAGE technique itself includes several steps utilizing molecular biological, DNA sequencing and bioinformatics techniques. These steps (reviewed in Adams MD, “Serial analysis of gene expression: ESTs get smaller.” Bioessays. 18(4):261-2 (1996)) have been used to produce 9 or 10 base “tags”, which are then, in some manner, assigned gene descriptions. For experimental reasons, these tags are immediately adjacent to the 3′ end of the 3′-most NlaIII restriction site in cDNA sequences. The Cancer Genome Anatomy Project, or CGAP, is an NCI-initiated and sponsored project, which hopes to delineate the molecular fingerprint of the cancer cell. It has created a database of those cancer-related projects that used SAGE analysis in order to gain insight into the initiation and development of cancer in the human body. The SAGE expression profiles reported in this invention are generated by first identifying the Unigene accession ID associated with the given MTC gene by querying the Unigene database at http://www.ncbi.nim.nih.gov/UniGene/. This page has then a link to the SAGE: Gene to Tag mapping (http://www.ncbi.nlm.nih.gov/SAGE/SAGEcid.cgi?cid=“unigeneID”).

This generated the reports that are included in this application, which list the number of tags found for the given gene in a given sample along with the relative expression. This information is then used to understand whether the gene has a more general role in tumorogenesis and/or tumor progression. A list of the SAGE libraries generated by CGAP and used in the analysis can be found at http://www.ncbi.nlm.nih.gov/SAGE/sagelb.cgi.

[image]

### Other Embodiments

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims.

What is claimed is:

1. An isolated polypeptide comprising an amino acid sequence selected from the group consisting of:
(a) a mature form of an amino acid sequence selected from the group consisting of SEQ ID NOS:2, 5, 7, 9, 11, 13,15, 17,19, 21, 23, 25, and 27;
(b) a variant of a mature form of an amino acid sequence selected from the group consisting of SEQ ID NOS:2, 5, 7, 9, 11, 13, 15, 17, 19, 21, 23, 25, and 27, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting SEQ ID NOS:2, 5, 7, 9, 11, 13, 15, 17, 19, 21, 23, 25, and 27; and
(d) a variant of an amino acid sequence selected from the group consisting of SEQ ID NOS:2, 5, 7, 9, 11, 13, 15, 17, 19, 21, 23, 25, and 27, wherein one or more amino acid residues in said variant differs from the amino acid sequence of said mature form, provided that said variant differs in no more than 15% of amino acid residues from said amino acid sequence.

2. The polypeptide of claim 1, wherein said polypeptide comprises the amino acid sequence of a naturally-occurring allelic variant of an amino acid sequence selected from the group consisting SEQ ID NOS:2, 5, 7, 9, 11, 13, 15, 17, 19, 21, 23, 25, and 27.

3. The polypeptide of claim 2, wherein said allelic variant comprises an amino acid sequence that is the translation of a nucleic acid sequence differing by a single nucleotide from a nucleic acid sequence selected from the group consisting of SEQ ID NOS: 1, 3, 4, 6, 8, 10, 12, 14, 16, 18, 20, 22, 24, and 26.

4. The polypeptide of claim 1, wherein the amino acid sequence of said variant comprises a conservative amino acid substitution.

5. An isolated nucleic acid molecule comprising a nucleic acid sequence encoding a polypeptide comprising an amino acid sequence selected from the group consisting of:
(a) a mature form of an amino acid sequence selected from the group consisting of SEQ ID NOS:2, 5, 7, 9, 11, 13, 15, 17, 19, 21, 23, 25, and 27;
(b) a variant of a mature form of an amino acid sequence selected from the group consisting of SEQ ID NOS:2, 5, 7, 9, 11, 13, 15, 17, 19, 21, 23, 25, and 27, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NOS:2, 5, 7, 9, 11, 13, 15, 17, 19, 21, 23, 25, and 27;
(d) a variant of an amino acid sequence selected from the group consisting SEQ ID NOS:2, 5, 7, 9, 11, 13, 15, 17, 19, 21, 23, 25, and 27, wherein one or more amino acid residues in said variant differs from the amino acid sequence of said mature form, provided that said variant differs in no more than 15% of amino acid residues from said amino acid sequence;
(e) a nucleic acid fragment encoding at least a portion of a polypeptide comprising an amino acid sequence chosen from the group consisting of SEQ ID NOS:2, 5, 7, 9, 11, 13, 15, 17, 19, 21, 23, 25, and 27, or a variant of said polypeptide, wherein one or more amino acid residues in said variant differs from the amino acid sequence of said mature form, provided that said variant differs in no more than 15% of amino acid residues from said amino acid sequence; and
(f) a nucleic acid molecule comprising the complement of (a), (b), (c), (d) or (e).

6. The nucleic acid molecule of claim 5, wherein the nucleic acid molecule comprises the nucleotide sequence of a naturally-occurring allelic nucleic acid variant.

7. The nucleic acid molecule of claim 5, wherein the nucleic acid molecule encodes a polypeptide comprising the amino acid sequence of a naturally-occurring polypeptide variant.

8. The nucleic acid molecule of claim 5, wherein the nucleic acid molecule differs by a single nucleotide from a nucleic acid sequence selected from the group consisting of SEQ ID NOS:1, 3, 4, 6, 8, 10, 12,14, 16, 18, 20, 22, 24, and 26.

9. The nucleic acid molecule of claim 5, wherein said nucleic acid molecule comprises a nucleotide sequence selected from the group consisting of:
(a) a nucleotide sequence selected from the group consisting of SEQ ID NOS: 1, 3, 4, 6, 8, 10, 12, 14, 16, 18, 20, 22, 24, and 26;
(b) a nucleotide sequence differing by one or more nucleotides from a nucleotide sequence selected from the group consisting of SEQ ID NOS:1, 3, 4, 6, 8, 10, 12, 14, 16, 18, 20, 22, 24, and 26, provided that no more than 20% of the nucleotides differ from said nucleotide sequence;
(c) a nucleic acid fragment of (a); and
(d) a nucleic acid fragment of (b).

10. The nucleic acid molecule of claim 5, wherein said nucleic acid molecule hybridizes under stringent conditions to a nucleotide sequence chosen from the group consisting SEQ ID NOS: 1, 3, 4, 6, 8, 10, 12, 14, 16, 18, 20, 22, 24, and 26, or a complement of said nucleotide sequence.

11. The nucleic acid molecule of claim 5, wherein the nucleic acid molecule comprises a nucleotide sequence selected from the group consisting of:
(a) a first nucleotide sequence comprising a coding sequence differing by one or more nucleotide sequences from a coding sequence encoding said amino acid sequence, provided that no more than 20% of the nucleotides in the coding sequence in said first nucleotide sequence differ from said coding sequence;
(b) an isolated second polynucleotide that is a complement of the first polynucleotide; and
(c) a nucleic acid fragment of (a) or (b).

12. A vector comprising the nucleic acid molecule of claim 11.

13. The vector of claim 12, further comprising a promoter operably-linked to said nucleic acid molecule.

14. A cell comprising the vector of claim 12.

15. An antibody that binds immunospecifically to the polypeptide of claim 1.

16. The antibody of claim 15, wherein said antibody is a monoclonal antibody.

17. The antibody of claim 15, wherein the antibody is a humanized antibody.

18. A method for determining the presence or amount of the polypeptide of claim 1 in a sample, the method comprising:
(a) providing the sample;
(b) contacting the sample with an antibody that binds immunospecifically to the polypeptide; and
(c) determining the presence or amount of antibody bound to said polypeptide, thereby determining the presence or amount of polypeptide in said sample.

19. A method for determining the presence or amount of the nucleic acid molecule of claim 5 in a sample, the method comprising:
(a) providing the sample;
(b) contacting the sample with a probe that binds to said nucleic acid molecule; and
(c) determining the presence or amount of the probe bound to said nucleic acid molecule,
thereby determining the presence or amount of the nucleic acid molecule in said sample.

20. The method of claim 19 wherein presence or amount of the nucleic acid molecule is used as a marker for cell or tissue type.

21. The method of claim 20 wherein the cell or tissue type is cancerous.

22. A method of identifying an agent that binds to a polypeptide of claim 1, the method comprising:
(a) contacting said polypeptide with said agent; and
(b) determining whether said agent binds to said polypeptide.

23. The method of claim 22 wherein the agent is a cellular receptor or a downstream effector.

24. A method for identifying an agent that modulates the expression or activity of the polypeptide of claim 1, the method comprising:
(a) providing a cell expressing said polypeptide;
(b) contacting the cell with said agent, and
(c) determining whether the agent modulates expression or activity of said polypeptide,
whereby an alteration in expression or activity of said peptide indicates said agent modulates expression or activity of said polypeptide.

25. A method for modulating the activity of the polypeptide of claim 1, the method comprising contacting a cell sample expressing the polypeptide of said claim with a compound that binds to said polypeptide in an amount sufficient to modulate the activity of the polypeptide.

26. A method of treating or preventing a NOVX-associated disorder, said method comprising administering to a subject in which such treatment or prevention is desired the polypeptide of claim 1 in an amount sufficient to treat or prevent said NOVX-associated disorder in said subject.

27. The method of claim 26 wherein the disorder is selected from the group consisting of cardiomyopathy and atherosclerosis.

28. The method of claim 26 wherein the disorder is related to cell signal processing and metabolic pathway modulation.

29. The method of claim 26, wherein said subject is a human.

30. A method of treating or preventing a NOVX-associated disorder, said method comprising administering to a subject in which such treatment or prevention is desired the nucleic acid of claim 5 in an amount sufficient to treat or prevent said NOVX-associated disorder in said subject.

31. The method of claim 30 wherein the disorder is selected from the group consisting of cardiomyopathy and atherosclerosis.

32. The method of claim 30 wherein the disorder is related to cell signal processing and metabolic pathway modulation.

33. The method of claim 30, wherein said subject is a human.

34. A method of treating or preventing a NOVX-associated disorder, said method comprising administering to a subject in which such treatment or prevention is desired the antibody of claim 15 in an amount sufficient to treat or prevent said NOVX-associated disorder in said subject.

35. The method of claim 34 wherein the disorder is diabetes.

36. The method of claim 34 wherein the disorder is related to cell signal processing and metabolic pathway modulation.

37. The method of claim 34, wherein the subject is a human.

38. A pharmaceutical composition comprising the polypeptide of claim 1 and a pharmaceutically-acceptable carrier.

39. A pharmaceutical composition comprising the nucleic acid molecule of claim 5 and a pharmaceutically-acceptable carrier.

40. A pharmaceutical composition comprising the antibody of claim 15 and a pharmaceutically-acceptable carrier.

41. A kit comprising in one or more containers, the pharmaceutical composition of claim 38.

42. A kit comprising in one or more containers, the pharmaceutical composition of claim 39.

43. A kit comprising in one or more containers, the pharmaceutical composition of claim 40.

44. A method for determining the presence of or predisposition to a disease associated with altered levels of the polypeptide of claim 1 in a first mammalian subject, the method comprising:
(a) measuring the level of expression of the polypeptide in a sample from the first mammalian subject; and
(b) comparing the amount of said polypeptide in the sample of step (a) to the amount of the polypeptide present in a control sample from a second mammalian subject known not to have, or not to be predisposed to, said disease;
wherein an alteration in the expression level of the polypeptide in the first subject as compared to the control sample indicates the presence of or predisposition to said disease.

45. The method of claim 44 wherein the predisposition is to a cancer.

46. A method for determining the presence of or predisposition to a disease associated with altered levels of the nucleic acid molecule of claim 5 in a first mammalian subject, the method comprising:
(a) measuring the amount of the nucleic acid in a sample from the first mammalian subject; and
(b) comparing the amount of said nucleic acid in the sample of step (a) to the amount of the nucleic acid present in a control sample from a second mammalian subject known not to have or not be predisposed to, the disease; wherein an alteration in the level of the nucleic acid in the first subject as compared to the control sample indicates the presence of or predisposition to the disease.

47. The method of claim 46 wherein the predisposition is to a cancer.

48. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising an amino acid sequence of at least one of SEQ ID NOS:2, 5, 7, 9, 11, 13, 15, 17, 19, 21, 23, 25, and 27, or a biologically active fragment thereof.

49. A method of treating a pathological state in a mammal, the method comprising administering to the mammal the antibody of claim 15 in an amount sufficient to alleviate the pathological state.


SEQUENCE LISTING (127 sequences)

SEQ ID NO: 1 - DNA, 2838 bp, Homo sapiens
aactttatgaagctatgggacttgacaaaaagtgatatttgagaagaaagtacgcagtggttggtgttttcttttttttaataaaggaattgaattactttgaacacctcttccagctgtgcattacagataacgtcaggaagagtctctgctttacagaatcggatttcatcacatgacaacatgaagctgtggattcatctcttttattcatctctccttgcctgtatatctttacactcccaaactccagtgctctcatccagaggctcttgtgattctctttgcaattgtgaggaaaaagatggcacaatgctaataaattgtgaagcaaaaggtatcaagatggtatctgaaataagtgtgccaccatcacgacctttccaactaagcttattaaataacggcttgacgatgcttcacacaaatgacttttctgggcttaccaatgctatttcaatacaccttggatttaacaatattgcagatattgagataggtgcatttaatggccttggcctcctgaaacaacttcatatcaatcacaattctttagaaattcttaaagaggatactttccatggactggaaaacctggaattcctgcaagcagataacaattttatcacagtgattgaaccaagtgcctttagcaagctcaacagactcaaagtgttaattttaaatgacaatgctattgagagtcttcctccaaacatcttccgatttgttcctttaacccatctagatcttcgtggaaatcaattacaaacattgccttatgttggttttctcgaacacattggccgaatattggatcttcagttggaggacaacaaatgggcctgcaattgtgacttattgcagttaaaaacttggttggagaacatgcctccacagtctataattggtgatgttgtctgcaacagccctccattttttaaaggaagtatactcagtagactaaagaaggaatctatttgccctactccaccagtgtatgaagaacatgaggatccttcaggatcattacatctggcagcaacatcttcaataaatgatagtcgcatgtcaactaagaccacgtccattctaaaactacccaccaaagcaccaggtttgataccttatattacaaagccatccactcaacttccaggaccttactgccctattccttgtaactgcaaagtcctatccccatcaggacttctaatacattgtcaggagcgcaacattgaaagcttatcagatctgagacctcctccgcaaaatcctagaaagctcattctagcgggaaatattattcacagtttaatgaagtctgatctagtggaatatttcactttggaaatgcttcacttgggaaacaatcgtattgaagttcttgaagaaggatcgtttatgaacctaacgagattacaaaaactctatctaaatggtaaccacctgaccaaattaagtaaaggcatgttccttggtctccataatcttgaatacttatatcttgaatacaatgccattaaggaaatactgccaggaacctttaatccaatgcctaaacttaaagtcctgtatttaaataacaacctcctccaagttttaccaccacatattttttcaggggttcctctaactaaggtaaatcttaaaacaaaccagtttacccatctacctgtaagtaatattttggatgatcttgatttactaacccagattgaccttgaggataacccctgggactgctcctgtgacctggttggactgcagcaatggatacaaaagttaagcaagaacacagtgacagatgacatcctctgcacttcccccgggcatctcgacaaaaaggaattgaaagccctaaatagtgaaattctctgtccaggtttagtaaataacccatccatgccaacacagactagttaccttatggtcaccactcctgcaacaacaacaaatacggctgatactattttacgatctcttacggacgctgtgccactgtctgttctaatattgggacttctgattatgttcatcactattgttttctgtgctgcagggatagtggttcttgttcttcaccgcaggagaagatacaaaaagaaacaagtagatgagcaaatgagagacaacagtcctgtgcatcttcagtacagcatgtatggccataaaaccactcatcacactactgaaagaccctctgcctcactctatgaacagcacatggtgagccccatggttcatgtctatagaagtccatcctttggtccaaagcatctggaagaggaagaagagaggaatgagaaagaaggaagtgatgcaaaacatctccaaagaagtcttttggaacaggaaaatcattcaccactcacagggtcaaatatgaaatacaaaaccacgaaccaatcaacagaatttttatccttccaagatgccagctcattgtacagaaacattttagaaaaagaaagggaacttcagcaactgggaatcacagaatacctaaggaaaaacattgctcagctccagcctgatatggaggcacattatcctggagcccacgaagagctgaagttaatggaaacattaatgtactcacgtccaaggaaggtattagtggaacagacaaaaaatgagtattttgaacttaaagctaatttacatgctgaacctgactatttagaagtcctggagcagcaaacatagatggagagtttgagggctttcgcagaaatgctgtgattctgttttaagtccataccttgtaaattagtgccttacgtgagtgtgtcatccatcagaacctaagcacagcagtaaactatggagaaaaaa

SEQ ID NO: 2 - protein, 841 aa, Homo sapiens
MKLWIHLFYSSLLACISLHSQTPVLSSRGSCDSLCNCEEKDGTMLINCEAKGIKMVSEISVPPSRPFQLSLLNNGLTMLHTNDFSGLTNAISIHLGFNNIADIEIGAFNGLGLLKQLHINHNSLEILKEDTFHGLENLEFLQADNNFITVIEPSAFSKLNRLKVLILNDNAIESLPPNIFRFVPLTHLDLRGNQLQTLPYVGFLEHIGRILDLQLEDNKWACNCDLLQLKTWLENMPPQSIIGDVVCNSPPFFKGSILSRLKKESICPTPPVYEEHEDPSGSLHLAATSSINDSRMSTKTTSILKLPTKAPGLIPYITKPSTQLPGPYCPIPCNCKVLSPSGLLIHCQERNIESLSDLRPPPQNPRKLILAGNIIHSLMKSDLVEYFTLEMLHLGNNRIEVLEEGSFMNLTRLQKLYLNGNHLTKLSKGMFLGLHNLEYLYLEYNAIKEILPGTFNPMPKLKVLYLNNNLLQVLPPHIFSGVPLTKVNLKTNQFTHLPVSNILDDLDLLTQIDLEDNPWDCSCDLVGLQQWIQKLSKNTVTDDILCTSPGHLDKKELKALNSEILCPGLVNNPSMPTQTSYLMVTTPATTTNTADTILRSLTDAVPLSVLILGLLIMFITIVFCAAGIVVLVLHRRRRYKKKQVDEQMRDNSPVHLQYSMYGHKTTHHTTERPSASLYEQHMVSPMVHVYRSPSFGPKHLEEEEERNEKEGSDAKHLQRSLLEQENHSPLTGSNMKYKTTNQSTEFLSFQDASSLYRNILEKERELQQLGITEYLRKNIAQLQPDMEAHYPGAHEELKLMETLMYSRPRKVLVEQTKNEYFELKANLHAEPDYLEVLEQQT

SEQ ID NO: 3 - DNA, 2526 bp, Homo sapiens
atgaagctgtggattcatctcttttattcatctctccttgcctgtatatctttacactcccaaactccagtgctctcatccagaggctcttgtgattctctttgcaattgtgaggaaaaagatggcacaatgctaataaattgtgaagcaaaaggtatcaagatggtatctgaaataagtgtgccaccatcacgacctttccaactaagcttattaaataacggcttgacgatgcttcacacaaatgacttttctgggcttaccaatgctatttcaatacaccttggatttaacaatattgcagatattgagataggtgcatttaatggccttggcctcctgaaacaacttcatatcaatcacaattctttagaaattcttaaagaggatactttccatggactggaaaacctggaattcctgcaagcagataacaattttatcacagtgattgaaccaagtgcctttagcaagctcaacagactcaaagtgttaattttaaatgacaatgctattgagagtcttcctccaaacatcttccgatttgttcctttaacccatctagatcttcgtggaaatcaattacaaacattgccttatgttggttttctcgaacacattggccgaatattggatcttcagttggaggacaacaaatgggcctgcaattgtgacttattgcagttaaaaacttggttggagaacatgcctccacagtctataattggtgatgttgtctgcaacagccctccattttttaaaggaagtatactcagtagactaaagaaggaatctatttgccctactccaccagtgtatgaagaacatgaggatccttcaggatcattacatctggcagcaacatcttcaataaatgatagtcgcatgtcaactaagaccacgtccattctaaaactacccaccaaagcaccaggtttgataccttatattacaaagccatccactcaacttccaggaccttactgccctattccttgtaactgcaaagtcctatccccatcaggacttctaatacattgtcaggagcgcaacattgaaagcttatcagatctgagacctcctccgcaaaatcctagaaagctcattctagcgggaaatattattcacagtttaatgaagtctgatctagtggaatatttcactttggaaatgcttcacttgggaaacaatcgtattgaagttcttgaagaaggatcgtttatgaacctaacgagattacaaaaactctatctaaatggtaaccacctgaccaaattaagtaaaggcatgttccttggtctccataatcttgaatacttatatcttgaatacaatgccattaaggaaatactgccaggaacctttaatccaatgcctaaacttaaagtcctgtatttaaataacaacctcctccaagttttaccaccacatattttttcaggggttcctctaactaaggtaaatcttaaaacaaaccagtttacccatctacctgtaagtaatattttggatgatcttgatttgctaacccagattgaccttgaggataacccctgggactgctcctgtgacctggttggactgcagcaatggatacaaaagttaagcaagaacacagtgacagatgacatcctctgcacttcccccgggcatctcgacaaaaaggaattgaaagccctaaatagtgaaattctctgtccaggtttagtaaataacccatccatgccaacacagactagttaccttatggtcaccactcctgcaacaacaacaaatacggctgatactattttacgatctcttacggacgctgtgccactgtctgttctaatattgggacttctgattatgttcatcactattgttttctgtgctgcagggatagtggttcttgttcttcaccgcaggagaagatacaaaaagaaacaagtagatgagcaaatgagagacaacagtcctgtgcatcttcagtacagcatgtatggccataaaaccactcatcacactactgaaagaccctctgcctcactctatgaacagcacatggtgagccccatggttcatgtctatagaagtccatcctttggtccaaagcatctggaagaggaagaagagaggaatgagaaagaaggaagtgatgcaaaacatctccaaagaagtcttttggaacaggaaaatcattcaccactcacagggtcaaatatgaaatacaaaaccacgaaccaatcaacagaatttttatccttccaagatgccagctcattgtacagaaacattttagaaaaagaaagggaacttcagcaactgggaatcacagaatacctaaggaaaaacattgctcagctccagcctgatatggaggcacattatcctggagcccacgaagagctgaagttaatggaaacattaatgtactcacgtccaaggaaggtattagtggaacagacaaaaaatgagtattttgaacttaaagctaatttacatgctgaacctgactatttagaagtcctggagcagcaaacatag

SEQ ID NO: 4 - DNA, 2531 bp, Homo sapiens
ggattctctcttttattcatctctccttgcctgtatatctttacactcccaaactccagtgctctcatccagaggctcttgtgattctctttgcaattgtgaggaaaaagatggcacaatgctaataaattgtgaagcaaaaggtatcaagatggtatctgaaataagtgtgctaccatcacgacctttccaactaagcttattaaataacggcttgacgatgcttcacacaaatgacttttctgggcttaccaatgctatttcaatacaccttggatttaacaatattgcagatattgagataggtgcatttaatggccttggcctcctgaaacaacttcatatcaatcacaattctttagaaattcttaaagaggatactttccatggactggaaaacctggaattcctgcaagcagataacaattttatcacagtgattgaaccaagtgcctttagcaagctcaacagactcaaagtgttaattttaaatgacaatgctattgagagtcttcctccaaacatcttccgatttgttcctttaacccatctagatcttcgtggaaatcaattacaaacattgccttatgttggttttctcgaacacattggccgaatattggatcttcagttggaggacaacaaatgggcctgcaattgtgacttattgcagttaaaaacttggttggagaacatgcctccacagtctataattggtgatgttgtctgcaacagccctccattttttaaaggaagtatactcagtagactaaagaaggaatctatttgccctactccaccagtgtatgaagaacatgaggatccttcaggatcattacatctggcagcaacatcttcaataaatgatagtcgcatgtcaactaagaccacgtccattctaaaactacccaccaaagcaccaggtttgataccttatattacaaagccatccactcaacttccaggaccttactgccctattccttgtaactgcaaagtcctatccccatcaggacttctaatacattgtcaggagcgcaacattgaaagcttatcagatctgagacctcctccgcaaaatcctagaaagctcattctagcgggaaatattattcacagtttaatgaagtctgatctagtggaatatttcactttggaaatgcttcacttgggaaacaatcgtattgaagttcttgaagaaggatcgtttatgaacctaacgagattacaaaaactctatctaaatggtaaccacctgaccaaattaagtaaaggcatgttccttggtctccataatcttgaatacttatatcttgaatacaatgccattaaggaaatactgccaggaacctttaatccaatgcctaaacttaaagtcctgtatttaaataacaacctcctccaagttttaccaccacatattttttcaggggttcctctaactaaggtaaatcttaaaacaaaccagtttacccatctacctgtaagtaatattttggatgatcttgatttgctaacccagattgaccttgaggataacccctgggactgctcctgtgacctggttggactgcagcaatggatacaaaagttaagcaagaacacagtgacagatgacatcctctgcacttcccccgggcatctcgacaaaaaggaattgaaagccctaaatagtgaaattctctgtccaggtttagtaaataacccatccatgccaacacagactagttaccttatggtcaccactcctgcaacaacaacaaatacggctgatactattttacgatctcttacggacgctgtgccactgtctgttctaatattgggacttctgattatgttcatcactattgttttctgtgctgcagggatagtggttcttgttcttcaccgcaggagaagatacaaaaagaaacaagtagatgagcaaatgagagacaacagtcctgtgcatcttcagtacagcatgtatggccataaaaccactcatcacactactgaaagaccctctgcctcactctatgaacagcacatggtgagccccatggttcatgtctatagaagtccatcctttggtccaaagcatctggaagaggaagaagagaggaatgagaaagaaggaagtgatgcaaaacatctccaaagaagtcttttggaacaggaaaatcattcaccactcacagggtcaaatatgaaatacaaaaccacgaaccaatcaacagaatttttatccttccaagatgccagctcattgtacagaaacattttagaaaaagaaagggaacttcagcaactgggaatcacagaatacctaaggaaaaacattgctcagctccagcctgatatggaggcacattatcctggagcccacgaagagctgaagttaatggaaacattaatgtactcacgtccaaggaaggtattagtggaacagacaaaaaatgagtattttgaacttaaagctaatttacatgctgaacctgactatttagaagtcctggagcagcaaacataagggcgaattctgctgt

SEQ ID NO: 5 - protein, 837 aa, Homo sapiens
DSLFYSSLLACISLHSQTPVLSSRGSCDSLCNCEEKDGTMLINCEAKGIKMVSEISVLPSRPFQLSLLNNGLTMLHTNDFSGLTNAISIHLGFNNIADIEIGAFNGLGLLKQLHINHNSLEILKEDTFHGLENLEFLQADNNFITVIEPSAFSKLNRLKVLILNDNAIESLPPNIFRFVPLTHLDLRGNQLQTLPYVGFLEHIGRILDLQLEDNKWACNCDLLQLKTWLENMPPQSIIGDVVCNSPPFFKGSILSRLKKESICPTPPVYEEHEDPSGSLHLAATSSINDSRMSTKTTSILKLPTKAPGLIPYITKPSTQLPGPYCPIPCNCKVLSPSGLLIHCQERNIESLSDLRPPPQNPRKLILAGNIIHSLMKSDLVEYFTLEMLHLGNNRIEVLEEGSFMNLTRLQKLYLNGNHLTKLSKGMFLGLHNLEYLYLEYNAIKEILPGTFNPMPKLKVLYLNNNLLQVLPPHIFSGVPLTKVNLKTNQFTHLPVSNILDDLDLLTQIDLEDNPWDCSCDLVGLQQWIQKLSKNTVTDDILCTSPGHLDKKELKALNSEILCPGLVNNPSMPTQTSYLMVTTPATTTNTADTILRSLTDAVPLSVLILGLLIMFITIVFCAAGIVVLVLHRRRRYKKKQVDEQMRDNSPVHLQYSMYGHKTTHHTTERPSASLYEQHMVSPMVHVYRSPSFGPKHLEEEEERNEKEGSDAKHLQRSLLEQENHSPLTGSNMKYKTTNQSTEFLSFQDASSLYRNILEKERELQQLGITEYLRKNIAQLQPDMEAHYPGAHEELKLMETLMYSRPRKVLVEQTKNEYFELKANLHAEPDYLEVLEQQT

SEQ ID NO: 6 - DNA, 3609 bp, Homo sapiens
cagtggatgcagaaggcagacagcagcaccgagacgatgaaggagaagaggacagcggctgcgatcaccgtgcggcacaggaccggctcctgcttctcgggccgctgtgtcaactccacctgcctctgcgacccgggctgggtgggggaccagtgccagcactgccagggcaggttcaggttaacagaaccttctggatatttaacagatggcccaattaactataaatataaaactaaatgtacttggctcattgaaggcccaaatgcagtgttaagattaagattcaatcattttgctacagaatgtagctgggatcatatgtatgtttatgatggagattcaatatatgcacctttaatagcttcttttagtggtttgatagtccctgaaataaggggcaatgaaactgtgcctgaagttgttactacatctggctatgcactgttacatttttttagtgatgctgcgtataatctaactggtttcaacattttctattcgatcaattcttgtcctaacaattgctctggtcatgggaagtgtacaactagtgtctctgttccaagtcaagtatattgtgaatgtgataaatactggaagggtgaagcttgtgatattccttactgtaaagccaattgcggcagtccagatcacggttactgtgacctgactggagaaaaattatgtgtctgcaatgatagttggcaaggtataggtcctgattgttctttgaatgttccctctactgagtcttactggattctgccaaacgttaaacccttcagtccttctgtaggtcgggcttcacataaagcagttttacacgggaaatttatgtgggtgattggtggatatacttttaactacagttcttttcaaatggtcctaagttacaatttagaaagcagtatatggaatgtaggaactccatcaaggggacctctccagagatatggacactctcttgctttatatcaggaaaacatctttatgtatggaggcagaattgaaacaaatgatggcaatgtcacagatgaattatgggtttttaacatacatagtcagtcatggagtacaaaaactcctactgttcttggacatggtcagcagtatgctgtggagggacattcagcacatattatggagttggatagtagagatgttgtcatgatcataatatttggatattctgcaatatatggttatacaagcagcatacaggaataccatatctgttcaaacacttggcttgttccagaaactaaaggagctattgtacaaggtggatatggccatactagtgtgtatgatgaaataacaaagtccatttatgttcatggagggtataaagcattgccagggaacaaatatggattggttgatgatctttataaatatgaagttaacactaagacttggactattttgaaagaaagtgggtttgccagataccttcattcagctgttcttatcaatggagctatgcttatttttggaggaaatacccataatgacacttccttgagtaacggtgcaaaatgtttttctgccgatttcctggcatatgacatatgcccaggctggagtgcagtggcacgatctcagctcactgccacctccacctcccacgttcaagcgattctcaataggtccatgtatatatttgggggattttctagtgtactccttaatgatatccttgtatacaagcctccaaattgcaaggctttcagagatgaagaactttgtaaaaatgctggtccagggataaaatgtgtttggaataaaaatcactgtgaatcttgggaatctgggaatactaataatattcttagagcaaagtgcttttctaaaagaaatctctgcagtgacagatgttacagatatgcagattgtgccagctgtactgccaatacaaatgggtgccaatggtgtgatgacaagaaatgcatttcggcaaatagtaactgcagtatggttagtatttttgggtatataaccttgccttcacagttcccattctattattgctacagatatgcagattgtgccagctgtactgccaatacaaatgggtgccaatggtgtgatgacaagaaatgcattgctttaccagctcatctttgtggagaaggatggagtcatattggggatgcttgtcttagagtcaattccagtagagaaaactatgacaatgcaaaactttattgctataatcttagtggaaatcttgcttcattaacaacctcaaaagaagtagaatttgttctggatgaaatacagaagtatacacaacagaaagtatcaccttgggtaggcttgcgcaagatcaatatatcctattggggatgggaagacatgtctccttttacaaacacaacactacagtggcttcctggcgaacccaatgattctgggttttgtgcatatctggaaagggctgcagtggcaggcttaaaagctaatccttgtacatctatggcaaatggccttgtctgtgaaaaacctgttaatcaaaatgcgaggccgtgcaaaaagccatgctctctgaggacatcatgttccaactgtacaagcaatggcatggagtgtatgtggtgcagcagtacgaaacgatgtgttgactctaatgcctatatcatctcttttccatatggacaatgtctagagtggcaaactgccacctgctcccgtgctcaaaattgttctggattgagaacctgtggacagtgtttggaacagcctgaatgtggctggtgcaatgatcctagtaatacaggaagaggacattgcattgaaggttcttcacggggaccaatgaagcttattggaatgcaccacagtgagatggttcttgacaccaatctttgccccaaagaaaagaactatgagtggtcctttatccagtgtccagcttgccagtgtaatggacatagcacttgcatcaataataatgtgtgcgaacagtgtaaaaatctcaccacaggaaagcagtgtcaagattgtatgccaggttattatggagatccaaccaatggtggacagtgcacagcttgtacatgcagtggccatgcaaatatctgtcatctgcacacaggaaaatgtttctgcacaactaaaggaataaaaggtgaccaatgccaattgtgtgactctgaaaatcgctatgttggtaatccacttagaggaacatgttattgtaagtatagccttttgattgattatcaatttaccttcagcttattacaggaagatgatcgccaccatactgccataaactttatagcaaacccagaacaggtgaggaaaaatctggatatatcaattaatgcatcaaacaactttaatctcaacattacgtggtctgtcggttcagctggaacaatatctggggaagagacttctatagtttccaagaataatataaaggaatacagagatagtttttcctatgaaaaatttaactttagaagcaatcctaacattacattctatgtgtacgtcagcaacttttcctggcctattaaaatacaggtaagtgttaagagtatttacttctaatgaccataatatcattaagaaaagaatggtgcttttgtccaaagt

SEQ ID NO: 7 - protein, 1185 aa, Homo sapiens
MQKADSSTETMKEKRTAAAITVRHRTGSCFSGRCVNSTCLCDPGWVGDQCQHCQGRFRLTEPSGYLTDGPINYKYKTKCTWLIEGPNAVLRLRFNHFATECSWDHMYVYDGDSIYAPLIASFSGLIVPEIRGNETVPEVVTTSGYALLHFFSDAAYNLTGFNIFYSINSCPNNCSGHGKCTTSVSVPSQVYCECDKYWKGEACDIPYCKANCGSPDHGYCDLTGEKLCVCNDSWQGIGPDCSLNVPSTESYWILPNVKPFSPSVGRASHKAVLHGKFMWVIGGYTFNYSSFQMVLSYNLESSIWNVGTPSRGPLQRYGHSLALYQENIFMYGGRIETNDGNVTDELWVFNIHSQSWSTKTPTVLGHGQQYAVEGHSAHIMELDSRDVVMIIIFGYSAIYGYTSSIQEYHICSNTWLVPETKGAIVQGGYGHTSVYDEITKSIYVHGGYKALPGNKYGLVDDLYKYEVNTKTWTILKESGFARYLHSAVLINGAMLIFGGNTHNDTSLSNGAKCFSADFLAYDICPGWSAVARSQLTATSTSHVQAILNRSMYIFGGFSSVLLNDILVYKPPNCKAFRDEELCKNAGPGIKCVWNKNHCESWESGNTNNILRAKCFSKRNLCSDRCYRYADCASCTANTNGCQWCDDKKCISANSNCSMVSIFGYITLPSQFPFYYCYRYADCASCTANTNGCQWCDDKKCIALPAHLCGEGWSHIGDACLRVNSSRENYDNAKLYCYNLSGNLASLTTSKEVEFVLDEIQKYTQQKVSPWVGLRKINISYWGWEDMSPFTNTTLQWLPGEPNDSGFCAYLERAAVAGLKANPCTSMANGLVCEKPVNQNARPCKKPCSLRTSCSNCTSNGMECMWCSSTKRCVDSNAYIISFPYGQCLEWQTATCSRAQNCSGLRTCGQCLEQPECGWCNDPSNTGRGHCIEGSSRGPMKLIGMHHSEMVLDTNLCPKEKNYEWSFIQCPACQCNGHSTCINNNVCEQCKNLTTGKQCQDCMPGYYGDPTNGGQCTACTCSGHANICHLHTGKCFCTTKGIKGDQCQLCDSENRYVGNPLRGTCYCKYSLLIDYQFTFSLLQEDDRHHTAINFIANPEQVRKNLDISINASNNFNLNITWSVGSAGTISGEETSIVSKNNIKEYRDSFSYEKFNFRSNPNITFYVYVSNFSWPIKIQVSVKSIYF

SEQ ID NO: 8 - DNA, 6201 bp, Homo sapiens
atgttgaagttcaaatatggagcgcggaatcctttggatgctggtgctgctgaacccattgccagccgggcctccaggctgaatctgttcttccaggggaaaccaccctttatgactcaacagcagatgtctcctctttcccgagaagggatattagatgccctctttgttctctttgaagaatgcagtcagcctgctctgatgaagattaagcacgtgagcaactttgtccggaagtgttccgacaccatagctgagttacaggagctccagccttcggcaaaggacttcgaagtcagaagtcttgtaggttgtggtcactttgctgaagtgcaggtggtaagagagaaagcaaccggggacatctatgctatgaaagtgatgaagaagaaggctttattggcccaggagcaggtttcattttttgaggaagagcggaacatattatctcgaagcacaagcccgtggatcccccaattacagtatgcctttcaggacaaaaatcacctttatctggtgatggaatatcagcctggaggggacttgctgtcacttttgaatagatatgaggaccagttagatgaaaacctgatacagttttacctagctgagctgattttggctgttcacagcgttcatctgatgggatacgtgcatcgggacatcaagcctgagaacattctcgttgaccgcacaggacacatcaagctggtggattttggatctgccgcgaaaatgaattcaaacaaggtgaatgccaaactcccgattgggaccccagattacatggctcctgaagtgctgactgtgatgaacggggatggaaaaggcacctacggcctggactgtgactggtggtcagtgggcgtgattgcctatgagatgatttatgggagatcccccttcgcagagggaacctctgccagaaccttcaataacattatgaatttccagcggtttttgaaatttccagatgaccccaaagtgagcagtgactttcttgatctgattcaaagcttgttgtgcggccagaaagagagactgaagtttgaaggtctttgctgccatcctttcttctctaaaattgactggaacaacattcgtaacgctcctccccccttcgttcccaccctcaagtctgacgatgacacctccaattttgatgaaccagagaagaattcgtgggtttcatcctctccgtgccagctgagcccctcaggcttctcgggtgaagaactgccgtttgtggggttttcgtacagcaaggcactggggattcttggtagatctgagtctgttgtgtcgggtctggactcccctgccaagactagctccatggaaaagaaacttctcatcaaaagcaaagagctacaagactctcaggacaagtgtcacaagatggagcaggaaatgacccggttacatcggagagtgtcagaggtggaggctgtgcttagtcagaaggaggtggagctgaaggcctctgagactcagagatccctcctggagcaggaccttgctacctacatcacagaatgcagtagcttaaagcgaagtttggagcaagcacggatggaggtgtcccaggaggatgacaaagcactgcagcttctccatgatatcagagagcagagccggaagctccaagaaatcaaagagcaggagtaccaggctcaagtggaagaaatgaggttgatgatgaatcagttggaagaggatcttgtctcagcaagaagacggagtgatctctacgaatctgagctgagagagtctcggcttgctgctgaagaattcaagcggaaagcgacagaatgtcagcataaactgttgaaggctaaggatcaggggaagcctgaagtgggagaatatgcgaaactggagaagatcaatgctgagcagcagctcaaaattcaggagctccaagagaaactggagaaggctgtaaaagccagcacggaggccaccgagctgctgcagaatatccgccaggcaaaggagcgagccgagagggagctggagaagctgcagaaccgagaggattcttctgaaggcatcagaaagaagctggtggaagctgaggaacgccgccattctctggagaacaaggtaaagagactagagaccatggagcgtagagaaaacagactgaaggatgacatccagacaaaatcccaacagatccagcagatggctgataaaattctggagctcgaagagaaacatcgggaggcccaagtctcagcccagcacctagaagtgcacctgaaacagaaagagcagcactatgaggaaaagattaaagtattggacaatcagataaagaaagacctggctgacaaggagacactggagaacatgatgcagagacacgaggaggaggcccatgagaagggcaaaattctcagcgaacagaaggcgatgatcaatgctatggattccaagatcagatccctggaacagaggattgtggaactgtctgaagccaataaacttgcagcaaatagcagtctttttacccaaaggaacatgaaggcccaagaagagatgatttctgaactcaggcaacagaaattttacctggagacacaggctgggaagttggaggcccagaaccgaaaactggaggagcagctggagaagatcagccaccaagaccacagtgacaagaatcggctgctggaactggagacaagattgcgggaggtgagtctagagcacgaggagcagaaactggagctcaagcgccagctcacagagctacagctctccctgcaggagcgcgagtcacagttgacagccctgcaggctgcacgggcggccctggagagccagcttcgccaggcgaagacagagctggaagagaccacagcagaagctgaagaggagatccaggcactcacggcacatagagatgaaatccagcgcaaatttgatgctcttcgtaacagctgtactgtgatcacagacctggaggagcagctaaaccagctgaccgaggacaacgctgaactcaacaaccaaaacttctacttgtccaaacaactcgatgaggcttctggcgccaacgacgagattgtacaactgcgaagtgaagtggaccatctccgccgggagatcacggaacgagagatgcagcttaccagccagaagcaaacgatggaggctctgaagaccacgtgcaccatgctggaggaacaggtcatggatttggaggccctaaacgatgagctgctagaaaaagagcggcagtgggaggcctggaggagcgtcctgggtgatgagaaatcccagtttgagtgtcgggttcgagagctgcagaggatgctggacaccgagaaacagagcagggcgagagccgatcagcggatcaccgagtctcgccaggtggtggagctggcagtgaaggagcacaaggctgagattctcgctctgcagcaggctctcaaagagcagaagctgaaggccgagagcctctctgacaagctcaatgacctggagaagaagcatgctatgcttgaaatgaatgcccgaagcttacagcagaagctggagactgaacgagagctcaaacagaggcttctggaagagcaagccaaattacagcagcagatggacctgcagaaaaatcacattttccgtctgactcaaggactgcaagaagctctagatcgggctgatctactgaagacagaaagaagtgacttggagtatcagctggaaaacattcaggtgctctattctcatgaaaaggtgaaaatggaaggcactatttctcaacaaaccaaactcattgattttctgcaagccaaaatggaccaacctgctaaaaagaaaaaggtgcctctgcagtacaatgagctgaagctggccctggagaaggagaaagctcgctgtgcagagctagaggaagcccttcagaagacccgcatcgagctccggtccgcccgggaggaagctgcccaccgcaaagcaacggaccacccacacccatccacgccagccaccgcgaggcagcagatcgccatgtctgccatcgtgcggtcgccagagcaccagcccagtgccatgagcctgctggccccgccatccagccgcagaaaggagtcttcaactccagaggaatttagtcggcgtcttaaggaacgcatgcaccacaatattcctcaccgattcaacgtaggactgaacatgcgagccacaaagtgtgctgtgtgtctggataccgtgcactttggacgccaggcatccaaatgtctagaatgtcaggtgatgtgtcaccccaagtgctccacgtgcttgccagccacctgcggcttgcctgctgaatatgccacacacttcaccgaggccttctgccgtgacaaaatgaactccccaggtctccagaccaaggagcccagcagcagcttgcacctggaagggtggatgaaggtgcccaggaataacaaacgaggacagcaaggctgggacaggaagtacattgtcctggagggatcaaaagtcctcatttatgacaatgaagccagagaagctggacagaggccggtggaagaatttgagctgtgccttcccgacggggatgtatctattcatggtgccgttggtgcttccgaactcgcaaatacagccaaagcagatgtcccatacatactgaagatggaatctcacccgcacaccacctgctggcccgggagaaccctctacttgctagctcccagcttccctgacaaacagcgctgggtcaccgccttagaatcagttgtcgcaggtgggagagtttctagggaaaaagcagaagctgatgctaaactgcttggaaactccctgctgaaactggaaggtgatgaccgtctagacatgaactgcacgctgcccttcagtgaccaggtagtgttggtgggcaccgaggaagggctctacgccctgaatgtcttgaaaaactccctaacccatgtcccaggaattggagcagtcttccaaatttatattatcaaggacctggagaagctactcatgatagcaggtgaagagcgggcactgtgtcttgtggacgtgaagaaagtgaaacagtccctggcccagtcccacctgcctgcccagcccgacatctcacccaacatttttgaagctgtcaagggctgccacttgtttggggcaggcaagattgagaacgggctctgcatctgtgcagccatgcccagcaaagtcgtcattctccgctacaacgaaaacctcagcaaatactgcatccggaaagagatagagacctcagagccctgcagctgtatccacttcaccaattacagtatcctcattggaaccaataaattctacgaaatcgacatgaagcagtacacgctcgaggaattcctggataagaatgaccattccttggcacctgctgtgtttgccgcctcttccaacagcttccctgtctcaatcgtgcaggtgaacagcgcagggcagcgagaggagtacttgctgtgtttccacgaatttggagtgttcgtggattcttacggaagacgtagccgcacagacgatctcaagtggagtcgcttacctttggcctttgcctacagagaaccctatctgtttgtgacccacttcaactcactcgaagtaattgagatccaggcacgctcctcagcagggacccctgcccgagcgtacctggacatcccgaacccgcgctacctgggccctgccatttcctcaggagcgatttacttggcgtcctcataccaggataaattaagggtcatttgctgcaagggaaacctcgtgaaggagtccggcactgaacaccaccggggcccgtccacctcccgcagcagccccaacaagcgaggcccacccacgtacaacgagcacatcaccaagcgcgtggcctccagcccagcgccgcccgaaggccccagccacccgcgagagccaagcacaccccaccgctaccgcgaggggcggaccgagctgcgcagggacaagtctcctggccgccccctggagcgagagaagtcccccggccggatgctcagcacgcggagagagcggtcccccgggaggctgtttgaagacagcagcaggggccggctgcctgcgggagccgtgaggaccccgctgtcccaggtgaacaaggtgaggcagcattccgaggcctgtgtgtctgttgcggaggccaggagtgacttggggaactga

SEQ ID NO: 9 - protein, 2066 aa, Homo sapiens
MLKFKYGARNPLDAGAAEPIASRASRLNLFFQGKPPFMTQQQMSPLSREGILDALFVLFEECSQPALMKIKHVSNFVRKCSDTIAELQELQPSAKDFEVRSLVGCGHFAEVQVVREKATGDIYAMKVMKKKALLAQEQVSFFEEERNILSRSTSPWIPQLQYAFQDKNHLYLVMEYQPGGDLLSLLNRYEDQLDENLIQFYLAELILAVHSVHLMGYVHRDIKPENILVDRTGHIKLVDFGSAAKMNSNKVNAKLPIGTPDYMAPEVLTVMNGDGKGTYGLDCDWWSVGVIAYEMIYGRSPFAEGTSARTFNNIMNFQRFLKFPDDPKVSSDFLDLIQSLLCGQKERLKFEGLCCHPFFSKIDWNNIRNAPPPFVPTLKSDDDTSNFDEPEKNSWVSSSPCQLSPSGFSGEELPFVGFSYSKALGILGRSESVVSGLDSPAKTSSMEKKLLIKSKELQDSQDKCHKMEQEMTRLHRRVSEVEAVLSQKEVELKASETQRSLLEQDLATYITECSSLKRSLEQARMEVSQEDDKALQLLHDIREQSRKLQEIKEQEYQAQVEEMRLMMNQLEEDLVSARRRSDLYESELRESRLAAEEFKRKATECQHKLLKAKDQGKPEVGEYAKLEKINAEQQLKIQELQEKLEKAVKASTEATELLQNIRQAKERAERELEKLQNREDSSEGIRKKLVEAEERRHSLENKVKRLETMERRENRLKDDIQTKSQQIQQMADKILELEEKHREAQVSAQHLEVHLKQKEQHYEEKIKVLDNQIKKDLADKETLENMMQRHEEEAHEKGKILSEQKAMINAMDSKIRSLEQRIVELSEANKLAANSSLFTQRNMKAQEEMISELRQQKFYLETQAGKLEAQNRKLEEQLEKISHQDHSDKNRLLELETRLREVSLEHEEQKLELKRQLTELQLSLQERESQLTALQAARAALESQLRQAKTELEETTAEAEEEIQALTAHRDEIQRKFDALRNSCTVITDLEEQLNQLTEDNAELNNQNFYLSKQLDEASGANDEIVQLRSEVDHLRREITEREMQLTSQKQTMEALKTTCTMLEEQVMDLEALNDELLEKERQWEAWRSVLGDEKSQFECRVRELQRMLDTEKQSRARADQRITESRQVVELAVKEHKAEILALQQALKEQKLKAESLSDKLNDLEKKHAMLEMNARSLQQKLETERELKQRLLEEQAKLQQQMDLQKNHIFRLTQGLQEALDRADLLKTERSDLEYQLENIQVLYSHEKVKMEGTISQQTKLIDFLQAKMDQPAKKKKVPLQYNELKLALEKEKARCAELEEALQKTRIELRSAREEAAHRKATDHPHPSTPATARQQIAMSAIVRSPEHQPSAMSLLAPPSSRRKESSTPEEFSRRLKERMHHNIPHRFNVGLNMRATKCAVCLDTVHFGRQASKCLECQVMCHPKCSTCLPATCGLPAEYATHFTEAFCRDKMNSPGLQTKEPSSSLHLEGWMKVPRNNKRGQQGWDRKYIVLEGSKVLIYDNEAREAGQRPVEEFELCLPDGDVSIHGAVGASELANTAKADVPYILKMESHPHTTCWPGRTLYLLAPSFPDKQRWVTALESVVAGGRVSREKAEADAKLLGNSLLKLEGDDRLDMNCTLPFSDQVVLVGTEEGLYALNVLKNSLTHVPGIGAVFQIYIIKDLEKLLMIAGEERALCLVDVKKVKQSLAQSHLPAQPDISPNIFEAVKGCHLFGAGKIENGLCICAAMPSKVVILRYNENLSKYCIRKEIETSEPCSCIHFTNYSILIGTNKFYEIDMKQYTLEEFLDKNDHSLAPAVFAASSNSFPVSIVQVNSAGQREEYLLCFHEFGVFVDSYGRRSRTDDLKWSRLPLAFAYREPYLFVTHFNSLEVIEIQARSSAGTPARAYLDIPNPRYLGPAISSGAIYLASSYQDKLRVICCKGNLVKESGTEHHRGPSTSRSSPNKRGPPTYNEHITKRVASSPAPPEGPSHPREPSTPHRYREGRTELRRDKSPGRPLEREKSPGRMLSTRRERSPGRLFEDSSRGRLPAGAVRTPLSQVNKVRQHSEACVSVAEARSDLGN

SEQ ID NO: 10 - DNA, 6189 bp, Homo sapiens
atgttgaagttcaaatatggagcgcggaatcctttggatgctggtgctgctgaacccattgccagccgggcctccaggctgaatctgttcttccaggggaaaccaccctttatgactcaacagcagatgtctcctctttcccgagaagggatattagatgccctctttgttctctttgaagaatgcagtcagcctgctctgatgaagattaagcacgtgagcaactttgtccggaagtgttccgacaccatagctgagttacaggagctccagccttcggcaaaggacttcgaagtcagaagtcttgtaggttgtggtcactttgctgaagtgcaggtggtaagagagaaagcaaccggggacatctatgctatgaaagtgatgaagaagaaggctttattggcccaggagcaggtttcattttttgaggaagagcggaacatattatctcgaagcacaagcccgtggatcccccaattacagtatgcctttcaggacaaaaatcacctttatctggtgatggaatatcagcctggaggggacttgctgtcacttttgaatagatatgaggaccagttagatgaaaacctgatacagttttacctagctgagctgattttggctgttcacagcgttcatctgatgggatacgtgcatcgggacatcaagcctgagaacattctcgttgaccgcacaggacacatcaagctggtggattttggatctgccgcgaaaatgaattcaaacaaggtgaatgccaaactcccgattgggaccccagattacatggctcctgaagtgctgactgtgatgaacggggatggaaaaggcacctacggcctggactgtgactggtggtcagtgggcgtgattgcctatgagatgatttatgggagatcccccttcgcagagggaacctctgccagaaccttcaataacattatgaatttccagcggtttttgaaatttccagatgaccccaaagtgagcagtgactttcttgatctgattcaaagcttgttgtgcggccagaaagagagactgaagtttgaaggtctttgctgccatcctttcttctctaaaattgactggaacaacattcgtaacgctcctccccccttcgttcccaccctcaagtctgacgatgacacctccaattttgatgaaccagagaagaattcgtgggtttcatcctctccgtgccagctgagcccctcaggcttctcgggtgaagaactgccgtttgtggggttttcgtacagcaaggcactggggattcttggtagatctgagtctgttgtgtcgggtctggactcccctgccaagactagctccatggaaaagaaacttctcatcaaaagcaaagagctacaagactctcaggacaagtgtcacaagatggagcaggaaatgacccggttacatcggagagtgtcagaggtggaggctgtgcttagtcagaaggaggtggagctgaaggcctctgagactcagagatccctcctggagcaggaccttgctacctacatcacagaatgcagtagcttaaagcgaagtttggagcaagcacggatggaggtgtcccaggaggatgacaaagcactgcagcttctccatgatatcagagagcagagccggaagctccaagaaatcaaagagcaggagtaccaggctcaagtggaagaaatgaggttgatgatgaatcagttggaagaggatcttgtctcagcaagaagacggagtgatctctacgaatctgagctgagagagtctcggcttgctgctgaagaattcaagcggaaagcgacagaatgtcagcataaactgttgaaggctaaggatcaggggaagcctgaagtgggagaatatgcgaaactggagaagatcaatgctgagcagcagctcaaaattcaggagctccaagagaaactggagaaggctgtaaaagccagcacggaggccaccgagctgctgcagaatatccgccaggcaaaggagcgagccgagagggagctggagaagctgcagaaccgagaggattcttctgaaggcatcagaaagaagctggtggaagctgaggaacgccgccattctctggagaacaaggtaaagagactagagaccatggagcgtagagaaaacagactgaaggatgacatccagacaaaatcccaacagatccagcagatggctgataaaattctggagctcgaagagaaacatcgggaggcccaagtctcagcccagcacctagaagtgcacctgaaacagaaagagcagcactatgaggaaaagattaaagtattggacaatcagataaagaaagacctggctgacaaggagacactggagaacatgatgcagagacacgaggaggaggcccatgagaagggcaaaattctcagcgaacagaaggcgatgatcaatgctatggattccaagatcagatccctggaacagaggattgtggaactgtctgaagccaataaacttgcagcaaatagcagtctttttacccaaaggaacatgaaggcccaagaagagatgatttctgaactcaggcaacagaaattttacctggagacacaggctgggaagttggaggcccagaaccgaaaactggaggagcagctggagaagatcagccaccaagaccacagtgacaagaatcggctgctggaactggagacaagattgcgggaggtgagtctagagcacgaggagcagaaactggagctcaagcgccagctcacagagctacagctctccctgcaggagcgcgagtcacagttgacagccctgcaggctgcacgggcggccctggagagccagcttcgccaggcgaagacagagctggaagagaccacagcagaagctgaagaggagatccaggcactcacggcacatagagatgaaatccagcgcaaatttgatgctcttcgtaacagctgtactgtgatcacagacctggaggagcagctaaaccagctgaccgaggacaacgctgaactcaacaaccaaaacttctacttgtccaaacaactcgatgaggcttctggcgccaacgacgagattgtacaactgcgaagtgaagtggaccatctccgccgggagatcacggaacgagagatgcagcttaccagccagaagcaaacgatggaggctctgaagaccacgtgcaccatgctggaggaacaggtcatggatttggaggccctaaacgatgagctgctagaaaaagagcggcagtgggaggcctggaggagcgtcctgggtgatgagaaatcccagtttgagtgtcgggttcgagagctgcagaggatgctggacaccgagaaacagagcagggcgagagccgatcagcggatcaccgagtctcgccaggtggtggagctggcagtgaaggagcacaaggctgagattctcgctctgcagcaggctctcaaagagcagaagctgaaggccgagagcctctctgacaagctcaatgacctggagaagaagcatgctatgcttgaaatgaatgcccgaagcttacagcagaagctggagactgaacgagagctcaaacagaggcttctggaagagcaagccaaattacagcagcagatggacctgcagaaaaatcacattttccgtctgactcaaggactgcaagaagctctagatcgggctgatctactgaagacagaaagaagtgacttggagtatcagctggaaaacattcaggtgctctattctcatgaaaaggtgaaaatggaaggcactatttctcaacaaaccaaactcattgattttctgcaagccaaaatggaccaacctgctaaaaagaaaaaggtgcctctgcagtacaatgagctgaagctggccctggagaaggagaaagctcgctgtgcagagctagaggaagcccttcagaagacccgcatcgagctccggtccgcccgggaggaagctgcccaccgcaaagcaacggaccacccacacccatccacgccagccaccgcgaggcagcagatcgccatgtctgccatcgtgcggtcgccagagcaccagcccagtgccatgagcctgctggccccgccatccagccgcagaaaggagtcttcaactccagaggaatttagtcggcgtcttaaggaacgcatgcaccacaatattcctcaccgattcaacgtaggactgaacatgcgagccacaaagtgtgctgtgtgtctggataccgtgcactttggacgccaggcatccaaatgtctagaatgtcaggtgatgtgtcaccccaagtgctccacgtgcttgccagccacctgcggcttgcctgctgaatatgccacacacttcaccgaggccttctgccgtgacaaaatgaactccccaggtctccagaccaaggagcccagcagcagcttgcacctggaagggtggatgaaggtgcccaggaataacaaacgaggacagcaaggctgggacaggaagtacattgtcctggagggatcaaaagtcctcatttatgacaatgaagccagagaagctggacagaggccggtggaagaatttgagctgtgccttcccgacggggatgtatctattcatggtgccgttggtgcttccgaactcgcaaatacagccaaagcagatgtcccatacatactgaagatggaatctcacccgcacaccacctgctggcccgggagaaccctctacttgctagctcccagcttccctgacaaacagcgctgggtcaccgccttagaatcagttgtcgcaggtgggagagtttctagggaaaaagcagaagctgatgctaaactgcttggaaactccctgctgaaactggaaggtgatgaccgtctagacatgaactgcacgctgcccttcagtgaccaggtagtgttggtgggcaccgaggaagggctctacgccctgaatgtcttgaaaaactccctaacccatgtcccaggaattggagcagtcttccaaatttatattatcaaggacctggagaagctactcatgatagcaggtgaagagcgggcactgtgtcttgtggacgtgaagaaagtgaaacagtccctggcccagtcccacctgcctgcccagcccgacatctcacccaacatttttgaagctgtcaagggctgccacttgtttggggcaggcaagattgagaacgggctctgcatctgtgcagccatgcccagcaaagtcgtcattctccgctacaacgaaaacctcagcaaatactgcatccggaaagagatagagacctcagagccctgcagctgtatccacttcaccaattacagtatcctcattggaaccaataaattctacgaaatcgacatgaagcagtacacgctcgaggaattcctggataagaatgaccattccttggcacctgctgtgtttgccgcctcttccaacagcttccctgtctcaatcgtgcaggtgaacagcgcagggcagcgagaggagtacttgctgtgtttccacgaatttggagtgttcgtggattcttacggaagacgtagccgcacagacgatctcaagtggagtcgcttacctttggcctttgcctacagagaaccctatctgtttgtgacccacttcaactcactcgaagtaattgagatccaggcacgctcctcagcagggacccctgcccgagcgtacctggacatcccgaacccgcgctacctgggccctgccatttcctcaggagcgatttacttggcgtcctcataccaggataaattaagggtcatttgctgcaagggaaacctcgtgaaggagtccggcactgaacaccaccggggcccgtccacctcccgcagcagccccaacaagcgaggcccacccacgtacaacgagcacatcaccaagcgcgtggcctccagcccagcgccgcccgaaggccccagccacccgcgagagccaagcacaccccaccgctaccgcgaggggcggaccgagctgcgcagggacaagtctcctggccgccccctggagcgagagaagtcccccggccggatgctcagcacgcggagagagcggtcccccgggaggctgtttgaagacagcagcaggggccggctgcctgcgggagccgtgaggaccccgctgtcccaggtgaacaaggtgtgggaccagtcttcagtataaatctcagccagaaaaaccaactcctca

SEQ ID NO: 11 - protein, 2053 aa, Homo sapiens
MLKFKYGARNPLDAGAAEPIASRASRLNLFFQGKPPFMTQQQMSPLSREGILDALFVLFEECSQPALMKIKHVSNFVRKCSDTIAELQELQPSAKDFEVRSLVGCGHFAEVQVVREKATGDIYAMKVMKKKALLAQEQVSFFEEERNILSRSTSPWIPQLQYAFQDKNHLYLVMEYQPGGDLLSLLNRYEDQLDENLIQFYLAELILAVHSVHLMGYVHRDIKPENILVDRTGHIKLVDFGSAAKMNSNKVNAKLPIGTPDYMAPEVLTVMNGDGKGTYGLDCDWWSVGVIAYEMIYGRSPFAEGTSARTFNNIMNFQRFLKFPDDPKVSSDFLDLIQSLLCGQKERLKFEGLCCHPFFSKIDWNNIRNAPPPFVPTLKSDDDTSNFDEPEKNSWVSSSPCQLSPSGFSGEELPFVGFSYSKALGILGRSESVVSGLDSPAKTSSMEKKLLIKSKELQDSQDKCHKMEQEMTRLHRRVSEVEAVLSQKEVELKASETQRSLLEQDLATYITECSSLKRSLEQARMEVSQEDDKALQLLHDIREQSRKLQEIKEQEYQAQVEEMRLMMNQLEEDLVSARRRSDLYESELRESRLAAEEFKRKATECQHKLLKAKDQGKPEVGEYAKLEKINAEQQLKIQELQEKLEKAVKASTEATELLQNIRQAKERAERELEKLQNREDSSEGIRKKLVEAEERRHSLENKVKRLETMERRENRLKDDIQTKSQQIQQMADKILELEEKHREAQVSAQHLEVHLKQKEQHYEEKIKVLDNQIKKDLADKETLENMMQRHEEEAHEKGKILSEQKAMINAMDSKIRSLEQRIVELSEANKLAANSSLFTQRNMKAQEEMISELRQQKFYLETQAGKLEAQNRKLEEQLEKISHQDHSDKNRLLELETRLREVSLEHEEQKLELKRQLTELQLSLQERESQLTALQAARAALESQLRQAKTELEETTAEAEEEIQALTAHRDEIQRKFDALRNSCTVITDLEEQLNQLTEDNAELNNQNFYLSKQLDEASGANDEIVQLRSEVDHLRREITEREMQLTSQKQTMEALKTTCTMLEEQVMDLEALNDELLEKERQWEAWRSVLGDEKSQFECRVRELQRMLDTEKQSRARADQRITESRQVVELAVKEHKAEILALQQALKEQKLKAESLSDKLNDLEKKHAMLEMNARSLQQKLETERELKQRLLEEQAKLQQQMDLQKNHIFRLTQGLQEALDRADLLKTERSDLEYQLENIQVLYSHEKVKMEGTISQQTKLIDFLQAKMDQPAKKKKVPLQYNELKLALEKEKARCAELEEALQKTRIELRSAREEAAHRKATDHPHPSTPATARQQIAMSAIVRSPEHQPSAMSLLAPPSSRRKESSTPEEFSRRLKERMHHNIPHRFNVGLNMRATKCAVCLDTVHFGRQASKCLECQVMCHPKCSTCLPATCGLPAEYATHFTEAFCRDKMNSPGLQTKEPSSSLHLEGWMKVPRNNKRGQQGWDRKYIVLEGSKVLIYDNEAREAGQRPVEEFELCLPDGDVSIHGAVGASELANTAKADVPYILKMESHPHTTCWPGRTLYLLAPSFPDKQRWVTALESVVAGGRVSREKAEADAKLLGNSLLKLEGDDRLDMNCTLPFSDQVVLVGTEEGLYALNVLKNSLTHVPGIGAVFQIYIIKDLEKLLMIAGEERALCLVDVKKVKQSLAQSHLPAQPDISPNIFEAVKGCHLFGAGKIENGLCICAAMPSKVVILRYNENLSKYCIRKEIETSEPCSCIHFTNYSILIGTNKFYEIDMKQYTLEEFLDKNDHSLAPAVFAASSNSFPVSIVQVNSAGQREEYLLCFHEFGVFVDSYGRRSRTDDLKWSRLPLAFAYREPYLFVTHFNSLEVIEIQARSSAGTPARAYLDIPNPRYLGPAISSGAIYLASSYQDKLRVICCKGNLVKESGTEHHRGPSTSRSSPNKRGPPTYNEHITKRVASSPAPPEGPSHPREPSTPHRYREGRTELRRDKSPGRPLEREKSPGRMLSTRRERSPGRLFEDSSRGRLPAGAVRTPLSQVNKVWDQSSV

SEQ ID NO: 12 - DNA, 5691 bp, Homo sapiens
atgaaagccatgccctggaactggacctgccttctctcccacctcctcatggtgggcatgggctcctccactttgctcacccggcagccagccccgctgtcccagaagcagcggtcatttgtcacattccgaggagagcccgccgagggtttcaatcacctggtggtggatgagaggacaggacacatttacttgggggccgtcaatcggatttacaagctctccagcgacctgaaggtcttggtgacgcatgagacagggccggacgaggacaaccccaagtgttacccaccccgcatcgtccagacctgcaatgagcccctgaccaccaccaacaatgtcaacaagatgctcctcatagactacaaggagaacaggctgattgcctgtgggagcctgtaccaaggcatctgcaagctgctgaggctggaggacctcttcaagctgggggagccttatcataagaaggagcactatctgtcaggtgtcaacgagagcggctcagtctttggagtgatcgtctcctacagcaacctggatgacaagctgttcattgccacggcagtggatgggaagcccgagtattttcccaccatctccagccggaaactgaccaagaactctgaggcggatggcatgttcgcgtacgtcttccatgatgagttcgtggcctcgatgattaagatcccttcggacaccttcaccatcatccctgactttgatatctactatgtctatggttttagcagtggcaactttgtctactttttgaccctccaacctgagatggtgtctccaccaggctccaccaccaaggagcaggtgtatacatccaagctcgtgaggctttgcaaggaggacacagccttcaactcctatgtagaggtgcccattggctgtgagcgcagtggggtggagtaccgcctgctgcaggctgcctacctgtccaaagcgggggccgtgcttggcaggacccttggagtccatccagatgatgacctgctcttcaccgtcttctccaagggccagaagcggaaaatgaaatccctggatgagtcggccctgtgcatcttcatcttgaagcagataaatgaccgcattaaggagcggctgcagtcttgttaccggggcgagggcacgctggacctggcctggctcaaggtgaaggacatcccctgcagcagtgcgctcttaaccattgacgataacttctgtggcctggacatgaatgctcccctgggagtgtccgacatggtgcgtggaattcccgtcttcacggaggacagggaccgcatgacgtctgtcatcgcatatgtctacaagaaccactctctggcctttgtgggcaccaaaagtggcaagctgaagaagatccgggtggatggacccaggggcaacgccctccagtatgagacggtgcaggtggtggaccccggcccagtcctccgggatatggccttctccaaggaccacgagcaactctacatcatgtcagagaggcagctcaccagagtccctgtggagtcctgtggtcagtatcagagctgcggcgagtgccttggctcaggcgacccccactgtggctggtgtgtgctgcacaacacgtgcacccggaaggagcggtgtgagcggtccaaggagccccgcaggtttgcctcggagatgaagcagtgtgtccggctgacggtccatcccaacaatatctccgtctctcagtacaacgtgctgctggtcctggagacgtacaatgtcccggagctgtcagctggcgtcaactgcacctttgaggacctgtcagagatggatgggctggtcgtgggcaatcagatccagtgctactcccctgcagccaaggaggtgccccggatcatcacagagaatggggaccaccatgtcgtacagcttcagctcaaatcaaaggagaccggcatgaccttcgccagcaccagctttgtcttctacaattgcagcgtccacaattcgtgcctgtcctgcgtggagagtccataccgctgccactggtgtaaataccggcatgtctgcacccatgaccccaagacctgctccttccaggaaggccgagtgaagctgcccgaggactgcccccagctgctgcgagtggacaagatcctggtgcccgtggaggtgatcaagcctatcacgctgaaggccaagaacctcccccagccccagtctgggcagcgtggctacgaatgcatcctcaacattcagggcagcgagcagcgagtgcccgccctgcgcttcaacagctccagcgtacagtgccagaacacctcttattcctatgaagggatggagatcaacaacctgcccgtggagttgacagtcgtgtggaatgggcacttcaacattgacaacccagctcagaataaagttcacctctacaagtgtggagccatgcgtgagagctgcgggctgtgcctcaaggctgacccagacttcgcatgtggctggtgccagggcccaggccagtgcaccctgcgccagcactgccctgcccaggagagccagtggctggagctgtctggtgccaaaagcaagtgcacaaacccccgcatcacagagataatcccggtgacaggcccccgggaagggggcaccaaggtcactatccgaggggagaacctgggcctggaatttcgcgacatcgcctcccatgtcaaggttgctggcgtggagtgcagccctttagtggatggttacatccctgcagaacagatcgtgtgtgagatgggggaggccaagcccagccagcatgcaggcttcgtggagatctgcgtggctgtgtgtcggcctgaattcatggcccggtcctcacagctctattacttcatgacactgactctctcagatctgaagcccagccgggggcccatgtccggagggacccaagtgaccatcacaggcaccaacctgaatgccggaagcaacgtggtggtgatgtttggaaagcagccctgtctcttccacaggcgatctccatcctacattgtctgcaacaccacatcctcagatgaggtgctagagatgaaggtgtcggtgcaggtggacagggccaagatccaccaggacctggtctttcagtatgtggaagaccccaccatcgtgcggattgagccagaatggagcattgtcagtggaaacacacccatcgccgtatgggggacccacctggacctcatacagaacccccagatccgtgccaagcatggagggaaggagcacatcaatatctgtgaggttctgaacgctactgagatgacctgtcaggcgcccgccctcgctctgggtcctgaccaccagtcagacctgaccgagaggcccgaggagtttggcttcatcctggacaacgtccagtccctgctcatcctcaacaagaccaacttcacctactatcccaacccggtgtttgaggcctttggtccctcaggaatcctggagctcaagcctggcacgcccatcatcctaaagggcaagaacctgatcccgcctgtggctgggggcaacgtgaagctgaactacactgtgctggttggggagaagccgtgcaccgtgaccgtgtcagatgtccagctgctctgcgagtcccccaacctcatcggcaggcacaaagtgatggcccgtgtcggtggcatggagtactccccggggatggtgtacattgccccggacagcccgctcagcctgcccgccatcgtcagcatcgcagtggctggcggcctcctcatcattttcatcgtggccgtgctcattgcctataaacgcaagtcccgcgaaagtgacctcacgctgaagcggctgcagatgcagatggacaacctggagtcccgtgtggccctggagtgcaaggaagcctttgccgagctgcagacggacatccatgagctgaccagtgacctggatggagccgggattccgttcctggactatagaacttacaccatgcgggtgctgttcccaggaattgaagaccaccctgtcctccgggaccttgaggtcccgggctaccggcaggagcgtgtggagaaaggcctgaagctcttcgcccagctcatcaacaacaaggtgttcctgctgtccttcatccgcacgcttgagtcccagcgtagcttctccatgcgcgaccgtggcaacgtggcctcactcatcatgaccgtgctgcagagcaagctggagtacgccactgatgtgctgaagcagctgctggccgacctcattgacaagaacctggagagcaagaaccaccctaagctgctgctcaggaggactgagtcagtggctgagaagatgctgaccaattggtttactttcctcctctacaagttcctcaaggagtgtgctggggagcccctcttctccctgttctgtgccatcaagcagcagatggagaagggccccattgacgccatcacgggcgaggcccgctactccttgagcgaggacaagctcatccgccagcagattgactacaaaaccctggtcctgagctgtgtcagcccagacaatgccaacagccccgaggtcccagtaaagatcctcaactgtgacaccatcactcaggtcaaggagaagattctggatgccatcttcaagaatgtgccttgctcccaccggcccaaagctgcagatatggatctggagtggcgacaaggaagtggggcaaggatgatcttgcaggatgaagacatcaccaccaagattgagaatgattggaagcgactgaacacactggcccactaccaggtgccagatggttccgtggtggcattagtgtccaagcaggtgacagcctataacgcagtgaacaactccaccgtctccaggacctcagcaagtaaatatgaaaacatgatccggtacacgggcagccccgacagcctccgctcacggacacctatgatcactcctgacctggagagtggagtcaagatgtggcacctagtgaagaaccacgagcacggagaccagaaggagggggaccgggggagcaagatggtgtctgaaatctacctgacccgactcctggccactaagggcacactgcagaagtttgtggatgacctctttgagaccatcttcagcacggcacaccgtggctctgccctgcccctggccatcaagtacatgtttgacttcctggatgagcaggctgataaacatggcattcatgacccgcacgtccgccatacctggaagagcaattgcctgcccctgaggttttgggtcaacatgatcaagaacccgcagtttgtgtttgacatccataagaacagcatcacagacgcctgcctctctgtggtggctcagaccttcatggactcttgctccacgtcagagcaccggctgggcaaggactcgccctccaacaagctgctgtatgccaaggacatccccagctacaagaattgggtggagaggtattactcagacatagggaagatgccagccatcagcgaccaagacatgaacgcatacctggctgagcagtcccggatgcacatgaatgagttcaacaccatgagtgcactctcagagatcttctcctatgtgggcaaatacagcgaggagatccttggacctctggaccacgatgaccagtgtgggaagcagaaactggcctacaaactagaacaagtcataaccctcatgagcttagacagctgaaataaa

SEQ ID NO: 13 - protein, 1896 aa, Homo sapiens
MKAMPWNWTCLLSHLLMVGMGSSTLLTRQPAPLSQKQRSFVTFRGEPAEGFNHLVVDERTGHIYLGAVNRIYKLSSDLKVLVTHETGPDEDNPKCYPPRIVQTCNEPLTTTNNVNKMLLIDYKENRLIACGSLYQGICKLLRLEDLFKLGEPYHKKEHYLSGVNESGSVFGVIVSYSNLDDKLFIATAVDGKPEYFPTISSRKLTKNSEADGMFAYVFHDEFVASMIKIPSDTFTIIPDFDIYYVYGFSSGNFVYFLTLQPEMVSPPGSTTKEQVYTSKLVRLCKEDTAFNSYVEVPIGCERSGVEYRLLQAAYLSKAGAVLGRTLGVHPDDDLLFTVFSKGQKRKMKSLDESALCIFILKQINDRIKERLQSCYRGEGTLDLAWLKVKDIPCSSALLTIDDNFCGLDMNAPLGVSDMVRGIPVFTEDRDRMTSVIAYVYKNHSLAFVGTKSGKLKKIRVDGPRGNALQYETVQVVDPGPVLRDMAFSKDHEQLYIMSERQLTRVPVESCGQYQSCGECLGSGDPHCGWCVLHNTCTRKERCERSKEPRRFASEMKQCVRLTVHPNNISVSQYNVLLVLETYNVPELSAGVNCTFEDLSEMDGLVVGNQIQCYSPAAKEVPRIITENGDHHVVQLQLKSKETGMTFASTSFVFYNCSVHNSCLSCVESPYRCHWCKYRHVCTHDPKTCSFQEGRVKLPEDCPQLLRVDKILVPVEVIKPITLKAKNLPQPQSGQRGYECILNIQGSEQRVPALRFNSSSVQCQNTSYSYEGMEINNLPVELTVVWNGHFNIDNPAQNKVHLYKCGAMRESCGLCLKADPDFACGWCQGPGQCTLRQHCPAQESQWLELSGAKSKCTNPRITEIIPVTGPREGGTKVTIRGENLGLEFRDIASHVKVAGVECSPLVDGYIPAEQIVCEMGEAKPSQHAGFVEICVAVCRPEFMARSSQLYYFMTLTLSDLKPSRGPMSGGTQVTITGTNLNAGSNVVVMFGKQPCLFHRRSPSYIVCNTTSSDEVLEMKVSVQVDRAKIHQDLVFQYVEDPTIVRIEPEWSIVSGNTPIAVWGTHLDLIQNPQIRAKHGGKEHINICEVLNATEMTCQAPALALGPDHQSDLTERPEEFGFILDNVQSLLILNKTNFTYYPNPVFEAFGPSGILELKPGTPIILKGKNLIPPVAGGNVKLNYTVLVGEKPCTVTVSDVQLLCESPNLIGRHKVMARVGGMEYSPGMVYIAPDSPLSLPAIVSIAVAGGLLIIFIVAVLIAYKRKSRESDLTLKRLQMQMDNLESRVALECKEAFAELQTDIHELTSDLDGAGIPFLDYRTYTMRVLFPGIEDHPVLRDLEVPGYRQERVEKGLKLFAQLINNKVFLLSFIRTLESQRSFSMRDRGNVASLIMTVLQSKLEYATDVLKQLLADLIDKNLESKNHPKLLLRRTESVAEKMLTNWFTFLLYKFLKECAGEPLFSLFCAIKQQMEKGPIDAITGEARYSLSEDKLIRQQIDYKTLVLSCVSPDNANSPEVPVKILNCDTITQVKEKILDAIFKNVPCSHRPKAADMDLEWRQGSGARMILQDEDITTKIENDWKRLNTLAHYQVPDGSVVALVSKQVTAYNAVNNSTVSRTSASKYENMIRYTGSPDSLRSRTPMITPDLESGVKMWHLVKNHEHGDQKEGDRGSKMVSEIYLTRLLATKGTLQKFVDDLFETIFSTAHRGSALPLAIKYMFDFLDEQADKHGIHDPHVRHTWKSNCLPLRFWVNMIKNPQFVFDIHKNSITDACLSVVAQTFMDSCSTSEHRLGKDSPSNKLLYAKDIPSYKNWVERYYSDIGKMPAISDQDMNAYLAEQSRMHMNEFNTMSALSEIFSYVGKYSEEILGPLDHDDQCGKQKLAYKLEQVITLMSLDSNK

SEQ ID NO: 14 - DNA, 1535 bp, Homo sapiens
cccgaaatgctgccgccaaggagcaacgacaccgcgtacccggggcagttagcgctataccagcagctggcgcaggggaatgccgtggggggctcggcgggggcaccgccactggggcccgtgcaggtggtcaccgcctgcctgctgaccctactcgtcatctggaccttgctgggcaacgtgctggtgtccgcagccatcgtgtggagccgccacctgcgcgccaagatgaccaacgtcttcatcgtgtctctacctgtgtcagacctcttcgtggcgctgctggtcatgtcctggaaggcagtcgccgaggtggccggttactggccctttgaagcgttctgcgacgtctgggtggccttcgacatcatgtgctccaccgcctccatcctgaacctgtgcgtcatcagcgtggcccgctactgggccatctccaggcccttccgctacgagcgcaagatgacccagcgcatggccttggtcatggtccgcccggcctggaccttgtccagcctcatctccttcattccggtccagctcaactggcacagggaccaggcggtctcttggggtgggctggacctgccaaacaacctggccaactggacgccctgggaggaggccgtttgggagcccgacgtgagggcagagaactgtgactccagcctgaatcgaacctacgccatcccttcctcgctcatcagcttctacatccccatggccatcatgatcgtgacctacacgcgcatctaccgcatcgcccaggtgcagatccgcaggatttcctccctggagagggccgcagagcacgtgcagagctgccggagcagcgcaggctgcacgcccgacaccagcctgcggttttccatcaagaaggagaccgaggttctcaagaccctgtcggtgatcatgggggtcttcgtgtgttgctggctgcccttcttcatccttaactgcatggttcctttctgcagtggacaccccaaaggccctccggccggcttcccctgcgtcagtgagaccacattcgatgtcttcatctggttctgctgggccaactcctcactcaacccagtccccagtcactatgccttcaacgccgacttccggaaggtgtttgcccagctgctggggtgcagccacgtctgctcccgcacgccggtggagacggtgaacatcagcaatgagctcatctcctacaaccaagacacggtcttccacaaggaaatcgcagctgcctacatccacatgatgcccaacgccattccccccggggaccgggaggtggacaacgatgaggaggaggagagtcctttcgatcgcatgtcccagatctatcagacatccccagatggtgaccatgttgcagagtctgtctgggagctggactgcgagggggagatttctttagacaaaataacacctttcaccccaaatggattccattaaactgcattaagaaaccccctcatggatctgcataaccacacagacattgacaagcatgcacacacaagcaaatacatggctttcca

SEQ ID NO: 15 - protein, 480 aa, Homo sapiens
MLPPRSNDTAYPGQLALYQQLAQGNAVGGSAGAPPLGPVQVVTACLLTLLVIWTLLGNVLVSAAIVWSRHLRAKMTNVFIVSLPVSDLFVALLVMSWKAVAEVAGYWPFEAFCDVWVAFDIMCSTASILNLCVISVARYWAISRPFRYERKMTQRMALVMVRPAWTLSSLISFIPVQLNWHRDQAVSWGGLDLPNNLANWTPWEEAVWEPDVRAENCDSSLNRTYAIPSSLISFYIPMAIMIVTYTRIYRIAQVQIRRISSLERAAEHVQSCRSSAGCTPDTSLRFSIKKETEVLKTLSVIMGVFVCCWLPFFILNCMVPFCSGHPKGPPAGFPCVSETTFDVFIWFCWANSSLNPVPSHYAFNADFRKVFAQLLGCSHVCSRTPVETVNISNELISYNQDTVFHKEIAAAYIHMMPNAIPPGDREVDNDEEEESPFDRMSQIYQTSPDGDHVAESVWELDCEGEISLDKITPFTPNGFH

SEQ ID NO: 16 - DNA, 2657 bp, Homo sapiens
gattcatgaagatgttgacaagactacaagttcttatgttagctttgttttcaaagggatttttagtctctttaggagatcacaactttatgaggagagaaattaaaatagaaggagaccttgttttagggggcttatttcctattaatgaaaaaggcactggaactgaagagtgtggacgaatcaatgaagacagaggtatccaacgcctggaggccatgttgtttgccattgatgaaatcaacaaagacaattacttgcttccaggagtgaagctgggggttcacattttggatacatgttcaagagacacctatgcattagagcagtcactggagtttgtcagagcatcgttgactaaagtggatgaagctgaatatatgtgtcctgatggatcatatgctattcaagaaaacatcccactactcattgcaggagtcattggcggttcgtacagcagtgtttccatacaggtagcaaacctgctgaggctcttccagatccctcagataagctacgcctccaccagtgccaaactcagcgacaaatcgcgctatgattattttgccaggaccgtgccccctgacttctaccaggccaaagccatggccgagatcttgcgctactttaactggacctatgtgtccactgttgcctctgaaggtgactatggggagacagggattgaggccttcgagcaggaagcaaggctacgcaacatctgcatcgccactgctgaaaaggtggggcgctccaacatccgcaagtcctacgacagcgtgatccgtgagctcctgcagaaacctaacgcgcgagttgtggtcctgttcatgcgcagtgatgactcacgagagttgatcgctgcagccagccgcgtgaatgcttccttcacctgggtggccagcgatggctggggtgcacaggagagcattgtcaagggcagtgagcacgtcgcctatggagccatcaccctggagctggcgtcccaccctgttcgtcagtttgatcgctacttccagagcctcaacccctacaacaatcatcgtaacccctggttccgagacttctgggagcagaagttccagtgcagcctccagaacaagagaaaccacagacagatttgtgacaagcacctggccattgacagcagcaactatgaacaagaatccaagatcatgtttgtggtgaatgcagtgtatgccatggcgcatgcgctgcacaaaatgcaacgcaccctctgtcccaacaccaccaagctctgtgatgcaatgaagatcctggatggaaagaagttgtacaaagattatttgctgaaaatcaacttccttgctccattcaacccaaataaaggagcagacagcattgtgaagtttgacacttacggagacgggatgggaagatacaacgtgttcaacttccagcatataggtggaaagtattcctacttaaaagttggccactgggcagaaactttatatctagatgtggactctattcattggtcccggaactcagtccccacttcccagtgcagtgatccctgtgcccccaatgaaatgaaaaacatgcagccaggagatgtttgctgctggatctgcatcccatgtgagccctatgaatacctggttgatgagttcacctgcatggattgtggccctggccagtggcccactgcagacctatctggatgctacaaccttccagaggattacatcaggtgggaagatgcctgggcaataggcccagtcactattgcctgcctgggttttatgtgtacatgcatagtcataactgtttttatcaagcacaacaacacacccttggtcaaagcatcaggccgagaactctgctacatcttgttatttggagttagcctgtcctattgcatgacattcttcttcattgctaagccatcgcctgtcatctgtgcattgcgccgacttgggcttgggacctcctttgccatctgttattcagctctcctgaccaagacaaactgcatcgctcgcatctttgatggggtcaagaatggcgctcagaggccaaaattcatcagccccagttctcaggtttttatctgcctgggtttgatactggtgcaaattgtgatggtgtctgtgtggcttatcttggagactccaggtactagaagatacaccctgccagagaagcgggaaacagtcatcctaaaatgcaatgtcaaagattccagcatgttgatctctctgacctatgacgtggttctggtgattctatgcactgtgtatgccttcaaaacaaggaagtgtcctgaaaacttcaatgaagccaagttcataggcttcaccatgtacaccacctgcatcatctggttggcattcctccctatattttatgtgacatcaagtgactacagagtacagacgacaacaatgtgcatctccgttagcttgagtggtttcgtggtcttgggctgtttgtttgcccccaaggtgcacattgtcctgttccaaccccagaagaatgtggtcacacacagacttcacctcaacaggttcagtgtcagtggaactgcgaccacatattctcaggcctctgcaagcacgtatgtgccaacggtgtgcaatgggcgggaagtcctcgactccaccacctcatctctgtgattgtgaattgca

SEQ ID NO: 17 - protein, 879 aa, Homo sapiens
MKMLTRLQVLMLALFSKGFLVSLGDHNFMRREIKIEGDLVLGGLFPINEKGTGTEECGRINEDRGIQRLEAMLFAIDEINKDNYLLPGVKLGVHILDTCSRDTYALEQSLEFVRASLTKVDEAEYMCPDGSYAIQENIPLLIAGVIGGSYSSVSIQVANLLRLFQIPQISYASTSAKLSDKSRYDYFARTVPPDFYQAKAMAEILRYFNWTYVSTVASEGDYGETGIEAFEQEARLRNICIATAEKVGRSNIRKSYDSVIRELLQKPNARVVVLFMRSDDSRELIAAASRVNASFTWVASDGWGAQESIVKGSEHVAYGAITLELASHPVRQFDRYFQSLNPYNNHRNPWFRDFWEQKFQCSLQNKRNHRQICDKHLAIDSSNYEQESKIMFVVNAVYAMAHALHKMQRTLCPNTTKLCDAMKILDGKKLYKDYLLKINFLAPFNPNKGADSIVKFDTYGDGMGRYNVFNFQHIGGKYSYLKVGHWAETLYLDVDSIHWSRNSVPTSQCSDPCAPNEMKNMQPGDVCCWICIPCEPYEYLVDEFTCMDCGPGQWPTADLSGCYNLPEDYIRWEDAWAIGPVTIACLGFMCTCIVITVFIKHNNTPLVKASGRELCYILLFGVSLSYCMTFFFIAKPSPVICALRRLGLGTSFAICYSALLTKTNCIARIFDGVKNGAQRPKFISPSSQVFICLGLILVQIVMVSVWLILETPGTRRYTLPEKRETVILKCNVKDSSMLISLTYDVVLVILCTVYAFKTRKCPENFNEAKFIGFTMYTTCIIWLAFLPIFYVTSSDYRVQTTTMCISVSLSGFVVLGCLFAPKVHIVLFQPQKNVVTHRLHLNRFSVSGTATTYSQASASTYVPTVCNGREVLDSTTSSL

SEQ ID NO: 18 - DNA, 1366 bp, Homo sapiens
atgggtctggccatggagcacggagggtcctacgctcgggcggggggcagctctcggggctgctggtattacctgcgctacttcttcctcttcgtctccctcatccaattcctcatcatcctggggctcgtgctcttcatggtctatggcaacgtgcacgtgagcacagagtccaacctgcaggccaccgagcgccgagccgagggcctatacagtcagctcctagggctcacggcctcccagtccaacttgaccaaggagctcaacttcaccacccgcgccaaggatgccatcatgcagatgtggctgaatgctcgccgcgacctggaccgcatcaatgccagcttccgccagtgccagggtgaccgggtaatctacacgaacaatcagaggtacatggctgccatcatcttgagtgagaagcaatgcagagatcaattcaaggacatgaacaagagctgcgatgccttgctcttcatgctgaatcagaaggtgaagacgctggaggtggagatagccaaggagaagaccatttgcactaaggataaggaaagcgtgctgctgaacaaacgcgtggcggaggaacagctggttgaatgcgtgaaaacccgggagctgcagcaccaagagcgccagctggccaaggagcaactgcaaaaggtgcaagccctctgcctgcccctggacaaggacaagtttgagatggaccttcgtaacctgtggagggactccattatcccacgcagcctggacaacctgggttacaacctctaccatcccctgggctcggaattggcctccatccgcagagcctgcgaccacatgcccagcctcatgagctccaaggtggaaggtcagtgccggagcctccgggcggatatcgaacgcgtggcccgcgagaactcagacctccaacgccagaagctggaagcccagcagggcctgcgggccagtcaggaggcgaaacagaaggtggagaaggaggctcaggcccgggaggccaagctccaagctgaatgctcccggcagacccagctagcgctggaggagaaggcggtgctgcggaaggaacgagacaacctggccaaggagctggaagagaagaagagggaggcggagcagctcaggatggagctggccatcagaaactcagccctggacacctgcatcaagaccaagtcgcagccgatgatgccagtgtcaaggcccatgggccctgtccccaacccccagcccatcgacccagctagcctggaggagttcaagaggaagatcctggagtcccagaggccccctgcaggcatccctgtagccccatccagtggctgaggaggctccggcactgacctaagggcgaatcccagca

SEQ ID NO: 19 - protein, 442 aa, Homo sapiens
MGLAMEHGGSYARAGGSSRGCWYYLRYFFLFVSLIQFLIILGLVLFMVYGNVHVSTESNLQATERRAEGLYSQLLGLTASQSNLTKELNFTTRAKDAIMQMWLNARRDLDRINASFRQCQGDRVIYTNNQRYMAAIILSEKQCRDQFKDMNKSCDALLFMLNQKVKTLEVEIAKEKTICTKDKESVLLNKRVAEEQLVECVKTRELQHQERQLAKEQLQKVQALCLPLDKDKFEMDLRNLWRDSIIPRSLDNLGYNLYHPLGSELASIRRACDHMPSLMSSKVEGQCRSLRADIERVARENSDLQRQKLEAQQGLRASQEAKQKVEKEAQAREAKLQAECSRQTQLALEEKAVLRKERDNLAKELEEKKREAEQLRMELAIRNSALDTCIKTKSQPMMPVSRPMGPVPNPQPIDPASLEEFKRKILESQRPPAGIPVAPSSG

SEQ ID NO: 20 - DNA, 1421 bp, Homo sapiens
gaattctaggtggtggtgagcagggacggtgcaccggacggcgggatcgagcaaatgggtctggccatggagtacggagggtcctacgctcgggcggggggcagctctcggggctgctggtattacctgcgctacttcttcctcttcgtctccctcatccaattcctcatcatcctggggctcgtgctcttcatggtctatggcgacgtgcacgtgagcacagagtccaacctgcaggccaccgagcgccgagccgagggcctatacagtcagctcctagggctcacggcctcccagtccaacttgaccaaggagctcaacttcaccacccgcgccaaggatgccatcatgcagatgtggctgaatgctcgccgcgacctggaccgcatcaatgccagcttccgccagtgccagggtgaccgggtcatctacacgaacaatcagaggtacatggctgccatcatcttgagtgagaagcaatgcagagatcaattcaaggacatgaacaagagctgcgatgccttgctcttcatgctgaatcagaaggtgaagacgctggaggtggagatagccaaggagaagaccatttgcactaaggataaggaaagcgtgctgctgaacaaacgcgtggcggaggaacagctggttgaatgcgtgaaaacccgggagctgcagcaccaagagcgccagctggccaaggagcaactgcaaaaggtgcaagccctctgcctgcccctggacaaggacaagtttgagatggaccttcgtaacctgtggagggactccattatcccacgcagcctggacaacctgggttacaacctctaccatcccctgggctcggaattggcctccatccgcagagcctgcgaccacatgcccagcctcatgagctccaaggtggaggagctggcccggagcctccgggcggatatcgaacgcgtggcccgcgagaactcagacctccaacgccagaagctggaagcccagcagggcctgcgggccagtcaggaggcgaaacagaaggtggagaaggaggctcaggcccgggaggccaagctccaagctgaatgctcccggcagacccagctagcgctggaggagaaggcggtgctgcggaaggaacgagacaacctggccaaggagctggaagagaagaagagggaggcggagcagctcaggatggagctggccatcagaaactcagccctggacacctgcatcaagaccaagtcgcagccgatgatgccagtgtcaaggcccatgggccctgtccccaacccccagcccatcgacccagctagcctggaggagttcaagaggaagatcctggagtcccagaggccccctgcaggcatccctgtagccccatccagtggctgaggaggctccaggcctgaggaccaagggatggcccgact

SEQ ID NO: 21 - protein, 442 aa, Homo sapiens
MGLAMEYGGSYARAGGSSRGCWYYLRYFFLFVSLIQFLIILGLVLFMVYGDVHVSTESNLQATERRAEGLYSQLLGLTASQSNLTKELNFTTRAKDAIMQMWLNARRDLDRINASFRQCQGDRVIYTNNQRYMAAIILSEKQCRDQFKDMNKSCDALLFMLNQKVKTLEVEIAKEKTICTKDKESVLLNKRVAEEQLVECVKTRELQHQERQLAKEQLQKVQALCLPLDKDKFEMDLRNLWRDSIIPRSLDNLGYNLYHPLGSELASIRRACDHMPSLMSSKVEELARSLRADIERVARENSDLQRQKLEAQQGLRASQEAKQKVEKEAQAREAKLQAECSRQTQLALEEKAVLRKERDNLAKELEEKKREAEQLRMELAIRNSALDTCIKTKSQPMMPVSRPMGPVPNPQPIDPASLEEFKRKILESQRPPAGIPVAPSSG

SEQ ID NO: 22 - DNA, 2024 bp, Homo sapiens
atgggtctggccatggagcacggagggtcctacgctcgggcggggggcagctctcggggctgctggtattacctgcgctacttcttcctcttcgtctccctcatccaattcctcatcatcctggggctcgtgctcttcatggtctatggcaacgtgcacgtgagcacagagtccaacctgcaggccaccgagcgccgagccgagggcctatacagtcagctcctagggctcacggcctcccagtccaacttgaccaaggagctcaacttcaccacccgcgccaaggatgccatcatgcagatgtggctgaatgctcgtcgcgacctggaccgcatcaatgccagcttccgccagtgccagggtgaccgggtcatctacacgaacaatcagaggtacatggctgccatcatcttgagtgagaagcaatgcagagatcaattcaaggacatgaacaagagctgcgatgccttgctcttcatgctgaatcagaaggtgaagacgctggaggtggagatagccaaggagaagaccatttgcactaaggataaggaaagcgtgctgctgaacaaacgcgtggcggaggaacagctggttgaatgcgtgaaaacccgggagctgcagcaccaagagcgccagctggccaaggagcaactgcaaagggtgcaagccctctgcctgcccctggacaaggacaagtttgagatggaccttcgtaacctgtggagggactccattatcccacgcagcctggacaacctgggttacaacctctaccatcccctgggctcggaattggcctccatccgcagagcctgcgaccacatgcccagcctcgtgagctccaaggtggaggagctggcccggagcctccgggcggatatcgaacgcgtggcccgcgagaactcagacctccaacgccagaagctggaagcccagcagggcctgcgggccagtcaggaggcgaaacagaaggtggagaaggaggctcaggcccgggaggccaagctccaagctgaatgctcccggcagacccagctagcgctggaggagaaggcggtgctgcggaaggaacgagacaacctggccaaggagctggaagagaagaagagggaggcggagcagctcaggatggagctggccatcagaaactcagccctggacacctgcatcaagaccaagtcgcagccgatgatgccagtgtcaaggcccatgggccctgtccccaacccccagcccatcgacccagctagcctggaggagttcaagaggaagatcctggagtcccagaggccccctgcaggcatccctgtagccccatccagtggctgaggaggctccaggcctgaggaccaagggatggcccgactcggcggtttgcggaggatgcagggatatgctcacagcgcccgacacaaccccctcccgccgcccccaaccacccagggccaccatcagacaactccctgcatgcaaacccctagtaccctctcacacccgcacccgcgcctcatgatccctcacccagagcacacggccgcggagatgacgtcacgcaagcaacggcgctgacgtcacatatcaccgtggtgatggcgtcacgtggccatgtagacgtcacgaagagatatagcgatggcgtcgtgcagatgcagcacgtcgcacacagacatggggaacttggcatgacgtcacaccgagatgcagcaacgacgtcacgggccatgtcgacgtcacacatattaatgtcacacagacgcggcgatggcatcacacagacggtgatgatgtcacacacagacacagtgacaacacacaccatgacaacgacacctatagatatggcaccaacatcacatgcacgcatgccctttcacacacactttctacccaattctcacctagtgtcacgttcccccgaccctggcacacgggccaaggtacccacaggatcccatcccctcccgcacagccctgggccccagcacctcccctcctccagcctcctggcctcccggtagtacacg

SEQ ID NO: 23 - protein, 442 aa, Homo sapiens
MGLAMEHGGSYARAGGSSRGCWYYLRYFFLFVSLIQFLIILGLVLFMVYGNVHVSTESNLQATERRAEGLYSQLLGLTASQSNLTKELNFTTRAKDAIMQMWLNARRDLDRINASFRQCQGDRVIYTNNQRYMAAIILSEKQCRDQFKDMNKSCDALLFMLNQKVKTLEVEIAKEKTICTKDKESVLLNKRVAEEQLVECVKTRELQHQERQLAKEQLQRVQALCLPLDKDKFEMDLRNLWRDSIIPRSLDNLGYNLYHPLGSELASIRRACDHMPSLVSSKVEELARSLRADIERVARENSDLQRQKLEAQQGLRASQEAKQKVEKEAQAREAKLQAECSRQTQLALEEKAVLRKERDNLAKELEEKKREAEQLRMELAIRNSALDTCIKTKSQPMMPVSRPMGPVPNPQPIDPASLEEFKRKILESQRPPAGIPVAPSSG

SEQ ID NO: 24 - DNA, 8640 bp, Homo sapiens
agctgatgatggccagggaccccaggggacgtggggccctgtggggtctggcccccaggagcaagacctctgatgatgctggtgtctgggagtgagcaccatgcccatcacccaggacaatgccgtgctgcacctgcccctcctctaccagtggctgcagaacagcctgcaggaaggtggggatgggccggagcagcggctctgccaggcggccatccagaagctgcaggagtacatccagctgaactttgctgtggatgagagtacggtcccacctgatcacagcccccccgaaatggagatctgtactgtgtacctcaccaaggagctgggggacacagagactgtgggcctgagttttgggaacatccctgttttcggggactatggtgaaaagcgcagggggggcaagaagaggaaaacccaccagggtcctgtgctggatgtgggctgcatctgggtgacagagctgaggaagaacagcccagcagggaagagtgggaaggtccgactgcgggatgagatcctctcactgaatgggcagctgatggttggagttgatgtcagtggggccagttacctggctgagcagtgctggaatggcggctttatctacctgatcatgctgcgtcgctttaagcacaaagcccactccacttataatggcaacagtagcaacagctctgaaccaggagaaacacctaccttggagctgggtgaccgaactgcgaaaaaggggaaacgaaccagaaagtttggggtcatctccaggcctcctgccaacaaggcccctgaagaatccaagggcagcgctggctgtgaggtgtccagtgaccccagcactgagctggagaacggcctggaccctgaacttggaaacggccatgtctttcagctagaaaatggcccagattctctcaaggaggtggctggaccccatctagagaggtcagaagtggacagagggacagagcatagaattccaaagacagatgctcctctgaccacaagcaatgacaaacgccgcttctcaaaaggtgggaagacggacttccaatcgagtgactgcctggcacggtccaaggaggaagttggccgaatatggaagatggagctgctcaaagaatcggatgggctgggaattcaggttagtggaggccgaggatcaaagcgctcacctcacgctatcgttgtcactcaagtgaaggaaggaggtgccgctcacaggctcagggatggcaggctgtccttaggagatgagctgctggtaatcaatggtcatttactggtcgggctctcccacgaggaagcagtggccattcttcgctccgccacgggaatggtgcagcttgtggtggccagcaaggtaggtgtgctttctgcatttcagatgcctgggacagatgaaccccaagatgtgtgcggtgctgaggaatccaaggggaacttggaaagtcccaaacagggcagcaataaaatcaagctcaagagtcgcctttcaggtaggtgggggctctacctgatgcagcctgtcgggggtgtacaccgccttgagtcagttgaagaatataacgagctgatggtgcggaatggggacccccggatccggatgttggaggtctcccgagatggccggaaacactccctcccgcagctgctggactcttccagtgcctcacaggaataccacattgtgaagaagtctacccgctccttaagcacgactcaggtggaatctccttggaggctcattcggccatccgtcatctcgatcattgggttgtacaaagaaaaaggcaagggccttggctttagtattgctggaggtcgagactgcattcgtggacagatggggatttttgtcaagaccatcttcccaaatggatcagctgcagaggacggaagacttaaagaaggtgatgaaatcctagatgtaaatggaataccaataaagggcttgacatttcaagaagccattcatacctttaagcaaatccggagtggattatttgttttaacggtacgcacaaagttggtgagccccagcctcacaccctgctcgacacccacacacatgagcagatccgcctccccgaacttcaataccagtgggggagcctcggcgggaggttccgatgaaggcagttcttcatccctgggtcggaagacccctgggcccaaggacaggatcgtcatggaagtaacactcaacaaagagccaagagttggattaggcattggtgcctgctgcttggctctggaaaacagtcctcctggcatctacattcacagccttgctccaggatcagtggccaagatggagagcaacctgtcgcggggatcaatcctggaagtgaactccgtcaacgtccgccatgctgctttaagcaaagtccacgccatcttgagtaaatgccctccaggacccgttcgccttgtcatcggccggcaccctaatccaaaggtgaatcaggtttccgagcaggaaatggatgaagtcatagcacgcagcacttatcaggagagcaaagaggccaattcctctcctggcttaggtactgtaatctcaatcggatgttttcttcttcaacaggactcccttatttctgaatctgaactctcccagtactttgcccacgatgtccctggccccttgtcagacttcatggtggccggttctgaggacgaggatcacccgggaagtggctgcagcacgtcggaggagggcagcctgcctcccagcacctccactcacaaggagcctggaaaacccagagccaacagcctcgtgactcttgggagccatcgggcttctgggctcttccacaagcaggtgacagttgccagacaagccagtctccccggaagcccacaggccctccgaaaccctctcctccgccagaggaaggtaggctgctacgatgccaacgatgccagtgatgaggaagagtttgacagagaaggggactgcatttcactcccaggggccctcccgggtcccatcaggcctctgtcagaggatgacccgaggcgtgtctcaatttcctcttccaagggcatggacgtccacaaccaagaggaacgaccccggaaaacactggtgagcaaggccatctcggcacctcttcttggtagctcagtggacttagaggagagtatcccagagggcatggtggatgctgcgtcctatgcagccaacctcacggactctgcagaggcccccaaggggagccctggaagctggtggaagaaggaactgtcaggatcaagtagcgcacccaaattggaatacacagtccgtacagacacccagagtccgacaaacactgggagccccagttccccccagcaaaaaagtgaaggcctgggctccaggcacagaccagtggccagggtaagcccccactgcaagagatccgaggctgaggccaagcccagtggctcacagacagtgaacctgactggcagagccaatgatccatgcgatctggactcgagagtccaggccacttctgtcaaagtgactgtcgctggctttcagccaggtggagctgtggagaaggaatctctgggaaagctgaccactggagatgcttgtgtctctaccagctgtgaactagccagtgctctgtcccatctggatgccagccacctcacagagaacctgcccaaagctgcatcagagctggggcaacaacccatgactgaactggacagctcctcggacctcatctcttccccagggaagaagggggccgctcatcctgaccccagcaagacctctgtagacacagggaaagtcagtcggccagagaatcccagccagcctgcatcgcccagggtcgccaagtgcaaggccaggtctccagtcaggctcccccatgagggcagcccctccccaggggagaaagcagcggctccccctgactacagcaagactcgatcagcatcggaaaccagcacaccccacaataccaggagggtggctgccctcaggggagcgggacctggagcagagggaatgacaccagctggtgctgtcctgccaggagaccccctcacatcccaggagcagagacagggagctccaggtaaccacagtaaggctctggaaatgacaggaatccatgcacctgaaagctcccaggagccttccctgctggagggagcagattctgtgtcctcaagggcaccgcaggccagcctctccatgctgccatccactgacaacaccaaagaagcatgtggccatgtctcggggcactgctgcccgggggggagtagagagagccctgtgacggacattgacagcttcatcaaggagctggatgcttctgcagcaaggtctccgtcttcccagacgggggacagtggctctcaggagggcagtgctcagggccacccaccagccggggctggaggtgggagctcctgccgtgccgaaccagtcccggggggccagacctcctccccgaggagggcctgggctgctggtgcccccgcctacccacaatgggcctcccagccttcggttttagattcaattaatcccgacaaacattttactgtgaacaaaaactttctgagcaactactctagaaattttagcagttttcatgaagacagcacctccctatcaggcctgggtgacagcacggagccgtctctgtcatccatgtatggcgatgctgaggattcttcttctgaccctgagtcactcactgaagccccacgagcttctgccagggacggctggtcccctcctcgttcccgtgtgtctttgcacaaggaagatccttcggagtcagaagaggaacagattgagatttgttccacacgtggctgccccaatccaccctcgagtcctgctcatcttcccacccaggctgccatctgtcctgcctcagccaaagttctgtcattaaaatacagcactccgagagagtcggtggccagtccccgtgagaaggtcgcctgcttgccaggctcatacacttcaggcccagactcttcccagccatcatcactcttggagatgagctctcaggagcatgaaactcatgcggacataagcacttcacagaaccacaggccctcgtgtgcagaagaaaccacagaagtcaccagcgctagctcagccatggaaaacagtccgctgtctaaagtagccaggcattttcacagtccgcccatcattctcagctcccccaacatggtaaatggcttggaacatgacctgctagatgacgaaaccctgaatcaatacgaaacaagcattaatgcagctgccagtctgtcctccttcagtgtggatgtccctaagaatggagaatctgttttggaaaacctccacatctctgaaagtcaagacctggatgacttgctacagaaaccaaaaatgatcgctaggaggcccatcatggcctggtttaaagaaataaataaacataaccaaggcacacatttgaggagcaaaaccgagaaggaacaacctctaatgcctgccagaagtcccgactccaagattcagatggtgagttcaagccaaaaaaagggcgttactgtgcctcatagccctcctcagccgaaaacaaacctggaaaataaggacctgtctaagaagagtccggcagaaatgcttctgactaatggtcagaaggcaaagtgtggtccgaagctgaagaggctcagcctcaagggcaaggccaaagtcaactctgaggcccctgctgcgaatgctgtgaaggctggggggacggaccacaggaaacccttgatctcaccccagacctcccacaaaacactttctaaggcagtgtcacagcggctccatgtagccgaccacgaggaccctgacagaaacaccacagctgcccccaggtccccccagtgtgtgctggaaagcaagccacctcttgccacctctgggccactgaaaccctcagtgtctgacacgagcatcaggacatttgtctcgcccctgacctctcccaagcctgttcctgagcaaggcatgtggagcaggttccacatggctgtcctctctgaacccgacagaggttgcccaaccacccctaaatctcctaagtgtagagcagagggcagggcgccccgtgctgactccgggccggtgagtccggcagcgtctaggaacggcatgtccgtggcagggaacagacagagtgagccgcgcctggccagccatgtggcagcagacacagcccaacccaggccgactggcgaaaaaggaggcaacataatggccagcgatcgcctcgaaagaacaaaccagctgaaaatcgtggagatttctgctgaagcagtgtcagagactgtatgtggtaacaagccagctgaaagcgacagacggggagggtgcttggcccagggcaactgtcaggagaagagtgaaatcaggctctatcgccaggtcgcagaatcatccacaagtcatccatcctcactcccatctcatgcctcccaggcagagcaggaaatgtcacgatcattcagcatggcaaaactggcgtcctcctcctcctcccttcaaacagccattagaaaggcagaatactcccagggaaaatcaagcctgatgtcagactcccgaggggtgcccagaaacagcattccagggggcccctcgggggaggaccatctctacttcaccccaaggccagcgaccaggacctactccatgccagcccagttctcaagccattttggacgggagggtcaccccccacacagcctgggtcgctctcgggacagccaggtccctgtgacaagcagtgttgtccccgaggcaaaggcatccagaggtggtcttcccagcctggctaatggacagggcatatatagtgtaaagccgctgctggacacatcgaggaatcttccagccacagatgaaggggatatcatttcagtccaggagacgagctgcctagtcacagacaaaatcaaagtcaccagacgacactactgctatgagcagaactggccccatgaatctacctcatttttctctgtgaagcagcggatcaagtcttttgagaacctggccaatgctgaccggcctgtagccaagtccggggcttccccatttttgtcggtgagctccaagcctcccattgggaggcggtcttccggcagcattgtttccgggagcctgggccacccaggtgacgcagcagcaaggttgttgagacgcagcttgagttcctgcagcgaaaaccaaagcgaagccggcaccctcctgccccagatggccaagtctccctcaatcatgacactgaccatctctcggcagaacccaccagagaccagtagcaagggctctgattcggaactaaagaaatcacttggtcctttgggaattcccaccccaacgatgaccctggcttctcctgttaagaggaacaagtcctcggtacgccacacgcagccctcgcccgtgtcccgctccaagctccaggagctgagagccttgagcatgcctgaccttgacaagctctgcagcgaggattactcagcagggccgagcgccgtgctcttcaaaactgagctggagatcacccccaggaggtcacctggccctcctgctggaggcgtttcgtgtcccgagaagggcgggaacagggcctgtccaggaggaagtggccctaaaaccagtgctgctgagacacccagttcagccagtgatacgggtgaagctgcccaggatctgccttttagaagaagctggtcagttaatttggatcaacttctagtctcagcgggggaccagcaaagattacagtctgttttatcgtcagtgggatcgaaatctaccatcctaactctcattcaggaagcgaaagcacaatcagagaatgaagaagatgtttgcttcatagtcttgaatagaaaagaaggctcaggtctgggattcagtgtggcaggagggacagatgtggagccaaaatcaatcacggtccacagggtgttttctcagggggcggcttctcaggaagggactatgaaccgaggggatttccttctgtcagtcaacggcgcctcactggctggcttagcccacgggaatgtcctgaaggttctgcaccaggcacagctgcacaaagatgccctcgtggtcatcaagaaagggatggatcagcccaggccctctgcccggcaggagcctcccacagccaatgggaagggtttgctgtccagaaagaccatccccctggagcctggcattgggagaagtgtggctgtacacgatgctctgtgtgttgaagtgctgaagacctcggctgggctgggactgagtctggatgggggaaaatcatcggtgacgggagatgggcccttggtcattaaaagagtgtacaaaggtggtgcggctgaacaagctggaataatagaagctggagatgaaattcttgctattaatgggaaacctctggttgggctcatgcactttgatgcctggaatattatgaagtctgtcccagaaggacctgtgcagttattaattagaaagcataggaattcttcatgaattttaacaagaatcattttctcagttctcttctttctttagcaaatcagagtgacttctttaaaccacaggttgttgaaatggccaacactggt

SEQ ID NO: 25 - protein, 2814 aa, Homo sapiens
MPITQDNAVLHLPLLYQWLQNSLQEGGDGPEQRLCQAAIQKLQEYIQLNFAVDESTVPPDHSPPEMEICTVYLTKELGDTETVGLSFGNIPVFGDYGEKRRGGKKRKTHQGPVLDVGCIWVTELRKNSPAGKSGKVRLRDEILSLNGQLMVGVDVSGASYLAEQCWNGGFIYLIMLRRFKHKAHSTYNGNSSNSSEPGETPTLELGDRTAKKGKRTRKFGVISRPPANKAPEESKGSAGCEVSSDPSTELENGLDPELGNGHVFQLENGPDSLKEVAGPHLERSEVDRGTEHRIPKTDAPLTTSNDKRRFSKGGKTDFQSSDCLARSKEEVGRIWKMELLKESDGLGIQVSGGRGSKRSPHAIVVTQVKEGGAAHRLRDGRLSLGDELLVINGHLLVGLSHEEAVAILRSATGMVQLVVASKVGVLSAFQMPGTDEPQDVCGAEESKGNLESPKQGSNKIKLKSRLSGRWGLYLMQPVGGVHRLESVEEYNELMVRNGDPRIRMLEVSRDGRKHSLPQLLDSSSASQEYHIVKKSTRSLSTTQVESPWRLIRPSVISIIGLYKEKGKGLGFSIAGGRDCIRGQMGIFVKTIFPNGSAAEDGRLKEGDEILDVNGIPIKGLTFQEAIHTFKQIRSGLFVLTVRTKLVSPSLTPCSTPTHMSRSASPNFNTSGGASAGGSDEGSSSSLGRKTPGPKDRIVMEVTLNKEPRVGLGIGACCLALENSPPGIYIHSLAPGSVAKMESNLSRGSILEVNSVNVRHAALSKVHAILSKCPPGPVRLVIGRHPNPKVNQVSEQEMDEVIARSTYQESKEANSSPGLGTVISIGCFLLQQDSLISESELSQYFAHDVPGPLSDFMVAGSEDEDHPGSGCSTSEEGSLPPSTSTHKEPGKPRANSLVTLGSHRASGLFHKQVTVARQASLPGSPQALRNPLLRQRKVGCYDANDASDEEEFDREGDCISLPGALPGPIRPLSEDDPRRVSISSSKGMDVHNQEERPRKTLVSKAISAPLLGSSVDLEESIPEGMVDAASYAANLTDSAEAPKGSPGSWWKKELSGSSSAPKLEYTVRTDTQSPTNTGSPSSPQQKSEGLGSRHRPVARVSPHCKRSEAEAKPSGSQTVNLTGRANDPCDLDSRVQATSVKVTVAGFQPGGAVEKESLGKLTTGDACVSTSCELASALSHLDASHLTENLPKAASELGQQPMTELDSSSDLISSPGKKGAAHPDPSKTSVDTGKVSRPENPSQPASPRVAKCKARSPVRLPHEGSPSPGEKAAAPPDYSKTRSASETSTPHNTRRVAALRGAGPGAEGMTPAGAVLPGDPLTSQEQRQGAPGNHSKALEMTGIHAPESSQEPSLLEGADSVSSRAPQASLSMLPSTDNTKEACGHVSGHCCPGGSRESPVTDIDSFIKELDASAARSPSSQTGDSGSQEGSAQGHPPAGAGGGSSCRAEPVPGGQTSSPRRAWAAGAPAYPQWASQPSVLDSINPDKHFTVNKNFLSNYSRNFSSFHEDSTSLSGLGDSTEPSLSSMYGDAEDSSSDPESLTEAPRASARDGWSPPRSRVSLHKEDPSESEEEQIEICSTRGCPNPPSSPAHLPTQAAICPASAKVLSLKYSTPRESVASPREKVACLPGSYTSGPDSSQPSSLLEMSSQEHETHADISTSQNHRPSCAEETTEVTSASSAMENSPLSKVARHFHSPPIILSSPNMVNGLEHDLLDDETLNQYETSINAAASLSSFSVDVPKNGESVLENLHISESQDLDDLLQKPKMIARRPIMAWFKEINKHNQGTHLRSKTEKEQPLMPARSPDSKIQMVSSSQKKGVTVPHSPPQPKTNLENKDLSKKSPAEMLLTNGQKAKCGPKLKRLSLKGKAKVNSEAPAANAVKAGGTDHRKPLISPQTSHKTLSKAVSQRLHVADHEDPDRNTTAAPRSPQCVLESKPPLATSGPLKPSVSDTSIRTFVSPLTSPKPVPEQGMWSRFHMAVLSEPDRGCPTTPKSPKCRAEGRAPRADSGPVSPAASRNGMSVAGNRQSEPRLASHVAADTAQPRPTGEKGGNIMASDRLERTNQLKIVEISAEAVSETVCGNKPAESDRRGGCLAQGNCQEKSEIRLYRQVAESSTSHPSSLPSHASQAEQEMSRSFSMAKLASSSSSLQTAIRKAEYSQGKSSLMSDSRGVPRNSIPGGPSGEDHLYFTPRPATRTYSMPAQFSSHFGREGHPPHSLGRSRDSQVPVTSSVVPEAKASRGGLPSLANGQGIYSVKPLLDTSRNLPATDEGDIISVQETSCLVTDKIKVTRRHYCYEQNWPHESTSFFSVKQRIKSFENLANADRPVAKSGASPFLSVSSKPPIGRRSSGSIVSGSLGHPGDAAARLLRRSLSSCSENQSEAGTLLPQMAKSPSIMTLTISRQNPPETSSKGSDSELKKSLGPLGIPTPTMTLASPVKRNKSSVRHTQPSPVSRSKLQELRALSMPDLDKLCSEDYSAGPSAVLFKTELEITPRRSPGPPAGGVSCPEKGGNRACPGGSGPKTSAAETPSSASDTGEAAQDLPFRRSWSVNLDQLLVSAGDQQRLQSVLSSVGSKSTILTLIQEAKAQSENEEDVCFIVLNRKEGSGLGFSVAGGTDVEPKSITVHRVFSQGAASQEGTMNRGDFLLSVNGASLAGLAHGNVLKVLHQAQLHKDALVVIKKGMDQPRPSARQEPPTANGKGLLSRKTIPLEPGIGRSVAVHDALCVEVLKTSAGLGLSLDGGKSSVTGDGPLVIKRVYKGGAAEQAGIIEAGDEILAINGKPLVGLMHFDAWNIMKSVPEGPVQLLIRKHRNSS

SEQ ID NO: 26 - DNA, 8640 bp, Homo sapiens
agctgatgatggccagggaccccaggggacgtggggccctgtggggtctggcccccaggagcaagacctctgatgatgctggtgtctgggagtgagcaccatgcccatcacccaggacaatgccgtgctgcacctgcccctcctctaccagtggctgcagaacagcctgcaggaaggtggggatgggccggagcagcggctctgccaggcggccatccagaagctgcaggagtacatccagctgaactttgctgtggatgagagtacggtcccacctgatcacagcccccccgaaatggagatctgtactgtgtacctcaccaaggagctgggggacacagagactgtgggcctgagttttgggaacatccctgttttcggggactatggtgaaaagcgcagggggggcaagaagaggaaaacccaccagggtcctgtgctggatgtgggctgcatctgggtgacagagctgaggaagaacagcccagcagggaagagtgggaaggtccgactgcgggatgagatcctctcactgaatgggcagctgatggttggagttgatgtcagtggggccagttacctggctgagcagtgctggaatggcggctttatctacctgatcatgctgcgtcgctttaagcacaaagcccactccacttataatggcaacagtagcaacagctctgaaccaggagaaacacctaccttggagctgggtgaccgaactgcgaaaaaggggaaacgaaccagaaagtttggggtcatctccaggcctcctgccaacaaggcccctgaagaatccaagggcagcgctggctgtgaggtgtccagtgaccccagcactgagctggagaacggcctggaccctgaacttggaaacggccatgtctttcagctagaaaatggcccagattctctcaaggaggtggctggaccccatctagagaggtcagaagtggacagagggacagagcatagaattccaaagacagatgctcctctgaccacaagcaatgacaaacgccgcttctcaaaaggtgggaagacggacttccaatcgagtgactgcctggcacggtccaaggaggaagttggccgaatatggaagatggagctgctcaaagaatcggatgggctgggaattcaggttagtggaggccgaggatcaaagcgctcacctcacgctatcgttgtcactcaagtgaaggaaggaggtgccgctcacaggctcagggatggcaggctgtccttaggagatgagctgctggtaatcaatggtcatttactggtcgggctctcccacgaggaagcagtggccattcttcgctccgccacgggaatggtgcagcttgtggtggccagcaaggtaggtgtgctttctgcatttcagatgcctgggacagatgaaccccaagatgtgtgcggtgctgaggaatccaaggggaacttggaaagtcccaaacagggcagcaataaaatcaagctcaagagtcgcctttcaggtaggtgggggctctacctgatgcagcctgtcgggggtgtacaccgccttgagtcagttgaagaatataacgagctgatggtgcggaatggggacccccggatccggatgttggaggtctcccgagatggccggaaacactccctcccgcagctgctggactcttccagtgcctcacaggaataccacattgtgaagaagtctacccgctccttaagcacgactcaggtggaatctcctcggaggctcattcggccatccgtcatctcgatcattgggttgtacaaagaaaaaggcaagggccttggctttagtattgctggaggtcgagactgcattcgtggacagatggggatttttgtcaagaccatcttcccaaatggatcagctgcagaggacggaagacttaaagaaggggatgaaatcctagatgtaaatggaataccaataaagggcttgacatttcaagaagccattcatacctttaagcaaatccggagtggattatttgttttaacggtacgcacaaagttggtgagccccagcctcacaccctgctcgacacccacacacatgagcagatccgcctccccgaacttcaataccagtgggggagcctcggcgggaggttccgatgaaggcagttcttcatccctgggtcggaagacccctgggcccaaggacaggatcgtcatggaagtaacactcaacaaagagccaagagttggattaggcattggtgcctgctgcttggctctggaaaacagtcctcctggcatctacattcacagccttgctccaggatcagtggccaagatggagagcaacctgagccgcggggatcaaatcctggaagtgaactccgtcaacgtccgccatgctgctttaagcaaagtccacgccatcttgagtaaatgccctccaggacccgttcgccttgtcatcggccggcaccctaatccaaaggtttccgagcaggaaatggatgaagtcatagcacgcagcacttatcaggagagcaaagaggccaattcctctcctggcttaggtacccccttgaagagtccctctcttgcaaaaaaggactcccttatttctgaatctgaactctcccagtactttgcccacgatgtccctggccccttgtcagacttcatggtggtcggttctgaggacgaggatcacccgggaagtggctgcagcacgtcggaggagggcagcctgcctcccagcacctccactcacaaggagcctggaaaacccagagccaacagcctcgtgactcttgggagccatcgggcttctgggctcttccacaagcaggtgacagttgccagacaagccagtctccccggaagcccacaggccctccgaaaccctctcctccgccagaggaaggtaggctgctacgatgccaacgatgccagtgatgaggaagagtttgacagagaaggggactgcatttcactcccaggggccctcccgggtcccatcaggcctctgtcagaggatgacccgaggcgtgtctcaatttcctcttccaagggcatggacgtccacaaccaagaggaacgaccccggaaaacactggtgagcaaggccatctcggcacctcttcttggtagctcagtggacttagaggagagtatcccagagggcatggtggatgctgcgtcctatgcagccaacctcacggactctgcagaggcccccaaggggagccctggaagctggtggaagaaggaactgtcaggatcaagtagcgcacccaaattggaatacacagtccgtacagacacccagagtccgacaaacactgggagccccagttccccccagcaaaaaagtgaaggcctgggctccaggcacagaccagtggccagggtaagcccccactgcaagagatccgaggctgaggccaagcccagtggctcacagacagtgaacctgactggcagagccaatgatccatgcgatctggactcgagagtccaggccacttctgtcaaagtgactgtcgctggctttcagccaggtggagctgtggagaaggaatctctgggaaagctgaccactggagatgcttgtgtctctaccagctgtgaactagccagtgctctgtcccatctggatgccagccacctcacagagaacctgcccaaagctgcatcagagctggggcaacaacccatgactgaactggacagctcctcggacctcatctcttccccagggaagaagggggccgctcatcctgaccccagcaagacctctgtagacacagggaaagtcagtcggccagagaatcccagccagcctgcatcgcccagggtcgccaagtgcaaggccaggtctccagtcaggctcccccatgagggcagcccctccccaggggagaaagcagcggctccccctgactacagcaagactcgatcagcatcggaaaccagcacaccccacaataccaggagggtggctgccctcaggggagcgggacctggagcagagggaatgacaccagctggtgctgtcctgccaggagaccccctcacatcccaggagcagagacagggagctccaggtaaccacagtaaggctctggaaatgacaggaatccatgcacctgaaagctcccaggagccttccctgctggagggagcagattctgtgtcctcaagggcaccgcaggccagcctctccatgctgccatccactgacaacaccaaagaagcatgtggccatgtctcggggcactgctgcccgggggggagtagagagagccctgtgacggacattgacagcttcatcaaggagctggatgcttctgcagcaaggtctccgtcttcccagacgggggacagtggctctcaggagggcagtgctcagggccacccaccagccggggctggaggtgggagctcctgccgtgccgaaccagtcccggggggccagacctcctccccgaggagggcctgggctgctggtgcccccgcctacccacaatgggcctcccagccttcggttttagattcaattaatcccgacaaacattttactgtgaacaaaaactttctgagcaactactctagaaattttagcagttttcatgaagacagcacctccctatcaggcctgggtgacagcacggagccgtctctgtcatccatgtatggcgatgctgaggattcttcttctgaccctgagtcactcactgaagccccacgagcttctgccagggacggctggtcccctcctcgttcccgtgtgtctttgcacaaggaagatccttcggagtcagaagaggaacagattgagatttgttccacacgtggctgccccaatccaccctcgagtcctgctcatcttcccacccaggctgccatctgtcctgcctcagccaaagttctgtcattaaaatacagcactccgagagagtcggtggccagtccccgtgagaaggtcgcctgcttgccaggctcatacacttcaggcccagactcttcccagccatcatcactcttggagatgagctctcaggagcatgaaactcatgcggacataagcacttcacagaaccacaggccctcgtgtgcagaagaaaccacagaagtcaccagcgctagctcagccatggaaaacagtccgctgtctaaagtagccaggcattttcacagtccgcccatcattctcagctcccccaacatggtaaatggcttggaacatgacctgctagatgacgaaaccctgaatcaatacgaaacaagcattaatgcagctgccagtctgtcctccttcagtgtggatgtccctaagaatggagaatctgttttggaaaacctccacatctctgaaagtcaagacctggatgacttgctacagaaaccaaaaatgatcgctaggaggcccatcatggcctggtttaaagaaataaataaacataaccaaggcacacatttgaggagcaaaaccgagaaggaacaacctctaatgcctgccagaagtcccgactccaagattcagatggtgagttcaagccaaaaaaagggcgttactgtgcctcatagccctcctcagccgaaaacaaacctggaaaataaggacctgtctaagaagagtccggcagaaatgcttctgactaatggtcagaaggcaaagtgtggtccgaagctgaagaggctcagcctcaagggcaaggccaaagtcaactctgaggcccctgctgcgaatgctgtgaaggctggggggacggaccacaggaaacccttgatctcaccccagacctcccacaaaacactttctaaggcagtgtcacagcggctccatgtagccgaccacgaggaccctgacagaaacaccacagctgcccccaggtccccccagtgtgtgctggaaagcaagccacctcttgccacctctgggccactgaaaccctcagtgtctgacacgagcatcaggacatttgtctcgcccctgacctctcccaagcctgttcctgagcaaggcatgtggagcaggttccacatggctgtcctctctgaacccgacagaggttgcccaaccacccctaaatctcctaagtgtagagcagagggcagggcgccccgtgctgactccgggccggtgagtccggcagcgtctaggaacggcatgtccgtggcagggaacagacagagtgagccgcgcctggccagccatgtggcagcagacacagcccaacccaggccgactggcgaaaaaggaggcaacataatggccagcgatcgcctcgaaagaacaaaccagctgaaaatcgtggagatttctgctgaagcagtgtcagagactgtatgtggtaacaagccagctgaaagcgacagacggggagggtgcttggcccagggcaactgtcaggagaagagtgaaatcaggctctatcgccaggtcgcagaatcatccacaagtcatccatcctcactcccatctcatgcctcccaggcagagcaggaaatgtcacgatcattcagcatggcaaaactggcgtcctcctcctcctcccttcaaacagccattagaaaggcagaatactcccagggaaaatcaagcctgatgtcagactcccgaggggtgcccagaaacagcattccagggggcccctcgggggaggaccatctctacttcaccccaaggccagcgaccaggacctactccatgccagcccagttctcaagccattttggacgggagggtcaccccccacacagcctgggtcgctctcgggacagccaggtccctgtgacaagcagtgttgtccccgaggcaaaggcatccagaggtggtcttcccagcctggctaatggacagggcatatatagtgtaaagccgctgctggacacatcgaggaatcttccagccacagatgaaggggatatcatttcagtccaggagacgagctgcctagtcacagacaaaatcaaagtcaccagacgacactactgctatgagcagaactggccccatgaatctacctcatttttctctgtgaagcagcggatcaagtcttttgagaacctggccaatgctgaccggcctgtagccaagtccggggcttccccatttttgtcggtgagctccaagcctcccattgggaggcggtcttccggcagcattgtttccgggagcctgggccacccaggtgacgcagcagcaaggttgttgagacgcagcttgagttcctgcagcgaaaaccaaagcgaagccggcaccctcctgccccagatggccaagtctccctcaatcatgacactgaccatctctcggcagaacccaccagagaccagtagcaagggctctgattcggaactaaagaaatcacttggtcctttgggaattcccaccccaacgatgaccctggcttctcctgttaagaggaacaagtcctcggtacgccacacgcagccctcgcccgtgtcccgctccaagctccaggagctgagagccttgagcatgcctgaccttgacaagctctgcagcgaggattactcagcagggccgagcgccgtgctcttcaaaactgagctggagatcacccccaggaggtcacctggccctcctgctggaggcgtttcgtgtcccgagaagggcgggaacagggcctgtccaggaggaagtggccctaaaaccagtgctgctgagacacccagttcagccagtgatacgggtgaagctgcccaggatctgccttttagaagaagctggtcagttaatttggatcaacttctagtctcagcgggggaccagcaaagattacagtctgttttatcgtcagtgggatcgaaatctaccatcctaactctcattcaggaagcgaaagcacaatcagagaatgaagaagatgtttgcttcatagtcttgaatagaaaagaaggctcaggtctgggattcagtgtggcaggagggacagatgtggagccaaaatcaatcacggtccacagggtgttttctcagggggcggcttctcaggaagggactatgaaccgaggggatttccttctgtcagtcaacggcgcctcactggctggcttagcccacgggaatgtcctgaaggttctgcaccaggcacagctgcacaaagatgccctcgtggtcatcaagaaagggatggatcagcccaggccctctgcccggcaggagcctcccacagccaatgggaagggtttgctgtccagaaagaccatccccctggagcctggcattgggagaagtgtggctgtacacgatgctctgtgtgttgaagtgctgaagacctcggctgggctgggactgagtctggatgggggaaaatcatcggtgacgggagatgggcccttggtcattaaaagagtgtacaaaggtggtgcggctgaacaagctggaataatagaagctggagatgaaattcttgctattaatgggaaacctctggttgggctcatgcactttgatgcctggaatattatgaagtctgtcccagaaggacctgtgcagttattaattagaaagcataggaattcttcatgaattttaacaagaatcattttctcagttctcttctttctttagcaaatcagagtgacttctttaaaccacaggttgttgaaatggccaacactggtacagacacg

SEQ ID NO: 27 - protein, 2811 aa, Homo sapiens
MPITQDNAVLHLPLLYQWLQNSLQEGGDGPEQRLCQAAIQKLQEYIQLNFAVDESTVPPDHSPPEMEICTVYLTKELGDTETVGLSFGNIPVFGDYGEKRRGGKKRKTHQGPVLDVGCIWVTELRKNSPAGKSGKVRLRDEILSLNGQLMVGVDVSGASYLAEQCWNGGFIYLIMLRRFKHKAHSTYNGNSSNSSEPGETPTLELGDRTAKKGKRTRKFGVISRPPANKAPEESKGSAGCEVSSDPSTELENGLDPELGNGHVFQLENGPDSLKEVAGPHLERSEVDRGTEHRIPKTDAPLTTSNDKRRFSKGGKTDFQSSDCLARSKEEVGRIWKMELLKESDGLGIQVSGGRGSKRSPHAIVVTQVKEGGAAHRLRDGRLSLGDELLVINGHLLVGLSHEEAVAILRSATGMVQLVVASKVGVLSAFQMPGTDEPQDVCGAEESKGNLESPKQGSNKIKLKSRLSGRWGLYLMQPVGGVHRLESVEEYNELMVRNGDPRIRMLEVSRDGRKHSLPQLLDSSSASQEYHIVKKSTRSLSTTQVESPRRLIRPSVISIIGLYKEKGKGLGFSIAGGRDCIRGQMGIFVKTIFPNGSAAEDGRLKEGDEILDVNGIPIKGLTFQEAIHTFKQIRSGLFVLTVRTKLVSPSLTPCSTPTHMSRSASPNFNTSGGASAGGSDEGSSSSLGRKTPGPKDRIVMEVTLNKEPRVGLGIGACCLALENSPPGIYIHSLAPGSVAKMESNLSRGDQILEVNSVNVRHAALSKVHAILSKCPPGPVRLVIGRHPNPKVSEQEMDEVIARSTYQESKEANSSPGLGTPLKSPSLAKKDSLISESELSQYFAHDVPGPLSDFMVVGSEDEDHPGSGCSTSEEGSLPPSTSTHKEPGKPRANSLVTLGSHRASGLFHKQVTVARQASLPGSPQALRNPLLRQRKVGCYDANDASDEEEFDREGDCISLPGALPGPIRPLSEDDPRRVSISSSKGMDVHNQEERPRKTLVSKAISAPLLGSSVDLEESIPEGMVDAASYAANLTDSAEAPKGSPGSWWKKELSGSSSAPKLEYTVRTDTQSPTNTGSPSSPQQKSEGLGSRHRPVARVSPHCKRSEAEAKPSGSQTVNLTGRANDPCDLDSRVQATSVKVTVAGFQPGGAVEKESLGKLTTGDACVSTSCELASALSHLDASHLTENLPKAASELGQQPMTELDSSSDLISSPGKKGAAHPDPSKTSVDTGKVSRPENPSQPASPRVAKCKARSPVRLPHEGSPSPGEKAAAPPDYSKTRSASETSTPHNTRRVAALRGAGPGAEGMTPAGAVLPGDPLTSQEQRQGAPGNHSKALEMTGIHAPESSQEPSLLEGADSVSSRAPQASLSMLPSTDNTKEACGHVSGHCCPGGSRESPVTDIDSFIKELDASAARSPSSQTGDSGSQEGSAQGHPPAGAGGGSSCRAEPVPGGQTSSPRRAWAAGAPAYPQWASQPSVLDSINPDKHFTVNKNFLSNYSRNFSSFHEDSTSLSGLGDSTEPSLSSMYGDAEDSSSDPESLTEAPRASARDGWSPPRSRVSLHKEDPSESEEEQIEICSTRGCPNPPSSPAHLPTQAAICPASAKVLSLKYSTPRESVASPREKVACLPGSYTSGPDSSQPSSLLEMSSQEHETHADISTSQNHRPSCAEETTEVTSASSAMENSPLSKVARHFHSPPIILSSPNMVNGLEHDLLDDETLNQYETSINAAASLSSFSVDVPKNGESVLENLHISESQDLDDLLQKPKMIARRPIMAWFKEINKHNQGTHLRSKTEKEQPLMPARSPDSKIQMVSSSQKKGVTVPHSPPQPKTNLENKDLSKKSPAEMLLTNGQKAKCGPKLKRLSLKGKAKVNSEAPAANAVKAGGTDHRKPLISPQTSHKTLSKAVSQRLHVADHEDPDRNTTAAPRSPQCVLESKPPLATSGPLKPSVSDTSIRTFVSPLTSPKPVPEQGMWSRFHMAVLSEPDRGCPTTPKSPKCRAEGRAPRADSGPVSPAASRNGMSVAGNRQSEPRLASHVAADTAQPRPTGEKGGNIMASDRLERTNQLKIVEISAEAVSETVCGNKPAESDRRGGCLAQGNCQEKSEIRLYRQVAESSTSHPSSLPSHASQAEQEMSRSFSMAKLASSSSSLQTAIRKAEYSQGKSSLMSDSRGVPRNSIPGGPSGEDHLYFTPRPATRTYSMPAQFSSHFGREGHPPHSLGRSRDSQVPVTSSVVPEAKASRGGLPSLANGQGIYSVKPLLDTSRNLPATDEGDIISVQETSCLVTDKIKVTRRHYCYEQNWPHESTSFFSVKQRIKSFENLANADRPVAKSGASPFLSVSSKPPIGRRSSGSIVSGSLGHPGDAAARLLRRSLSSCSENQSEAGTLLPQMAKSPSIMTLTISRQNPPETSSKGSDSELKKSLGPLGIPTPTMTLASPVKRNKSSVRHTQPSPVSRSKLQELRALSMPDLDKLCSEDYSAGPSAVLFKTELEITPRRSPGPPAGGVSCPEKGGNRACPGGSGPKTSAAETPSSASDTGEAAQDLPFRRSWSVNLDQLLVSAGDQQRLQSVLSSVGSKSTILTLIQEAKAQSENEEDVCFIVLNRKEGSGLGFSVAGGTDVEPKSITVHRVFSQGAASQEGTMNRGDFLLSVNGASLAGLAHGNVLKVLHQAQLHKDALVVIKKGMDQPRPSARQEPPTANGKGLLSRKTIPLEPGIGRSVAVHDALCVEVLKTSAGLGLSLDGGKSSVTGDGPLVIKRVYKGGAAEQAGIIEAGDEILAINGKPLVGLMHFDAWNIMKSVPEGPVQLLIRKHRNSS

SEQ ID NO: 28 - protein, 798 aa, Homo sapiens [sequence omitted]

SEQ ID NO: 29 - protein, 440 aa, Homo sapiens
MLINCEAKGIKMVSEISVPPSRPFQLSLLNNGLTMLHTNDFSGLTNAISIHLGFNNIADIEIGAFNGLGLLKQLHINHNSLEILKEDTFHGLENLEFLQADNNFITVIEPSAFSKLNRLKVLILNDNAIESLPPNIFRFVPLTHLDLRGNQLQTLPYVGFLEHIGRILDLQLEDNKWACNCDLLQLKTWLENMPPQSIIGDVVCNSPPFFKGSILSRLKKESICPTPPVYEEHEDPSGSLHLAATSSINDSRMSTKTTSILKLPTKAPGLIPYITKPSTQLPGPYCPIPCNCKVLSPSGLLIHCQERNIESLSDLRPPPQNPRKLILAGNIIHSLMKSDLVEYFTLEMLHLGNNRIEVLEEGSFMNLTRLQKLYLNGNHLTKLSKGMFLGLHNLEYLYLEYNAIKEILPGTFNPMPKLKVLYLNNTSSKFYHHIFFQGFL

SEQ ID NO: 30 - protein, 853 aa, Homo sapiens
YFSLFRSIQLFADCKKMFLWLFLILSALISSTNADSDISVEICNVCSCVSVENVLYVNCEKVSVYRPNQLKPPWSNFYHLNFQNNFLNILYPNTFLNFSHAVSLHLGNNKLQNIEGGAFLGLSALKQLHLNNNELKILRADTFLGIENLEYLQADYNLIKYIERGAFNKLHKLKVLILNDNLISFLPDNIFRFASLTHLDIRGNRIQKLPYIGVLEHIGRVVELQLEDNPWNCSCDLLPLKAWLENMPYNIYIGEAICETPSDLYGRLLKETNKQELCPMGTGSDFDVRILPPSQLENGYTTPNGHTTQTSLHRLVTKPPKTTNPSKISGIVAGKALSNRNLSQIVSYQTRVPPLTPCPAPCFCKTHPSDLGLSVNCQEKNIQSMSELIPKPLNAKKLHVNGNSIKDVDVSDFTDFEGLDLLHLGSNQITVIKGDVFHNLTNLRRLYLNGNQIERLYPEIFSGLHNLQYLYLEYNLIKEISAGTFDSMPNLQLLYLNNNLLKSLPVYIFSGAPLARLNLRNNKFMYLPVSGVLDQLQSLTQIDLEGNPWDCTCDLVALKLWVEKLSDGIVVKELKCETPVQFANIELKSLKNEILCPKLLNKPSAPFTSPAPAITFTTPLGPIRSPPGGPVPLSILILSILVVLILTVFVAFCLLVFVLRRNKKPTVKHEGLGNPDCGSMQLQLRKHDHKTNKKDGLSTEAFIPQTIEQMSKSHTCGLKESETGFMFSDPPGQKVVMRNVADKEKDLLHVDTRKRLSTIDELDELFPSRDSNVFIQNFLESKKEYNSIGVSGFEIRYPEKQPDKKSKKSLIGGNHSKIVVEQRKSEYFELKAKLQSSPDYLQVLEEQTALNKI

SEQ ID NO: 31 - protein, 314 aa, Homo sapiens
LQQWIQKLSKNTVTDDILCTSPGHLDKKELKALNSEILCPGLVNNPSMPTQTSYLMVTTPATTTNTADTILRSLTDAVPLSVLILGLLIMFITIVFCAAGIVVLVLHRRRRYKKKQVDEQMRDNSPVHLQYSMYGHKTTHHTTERPSASLYEQHMVSPMVHVYRSPSFGPKHLEEEEERNEKEGSDAKHLQRSLLEQENHSPLTGSNMKYKTTNQSTEFLSFQDASSLYRNILEKERELQQLGITEYLRKNIAQLQPDMEAHYPGAHEELKLMETLMYSRPRKVLVEQTKNEYFELKANLHAEPDYLEVLEQQT

SEQ ID NO: 32 - protein, 966 aa, Homo sapiens
RRGAQGGKMHTCCPPVTLEQDLHRKMHSWMLQTLAFAVTSLVLSCAETIDYYGEICDNACPCEEKDGILTVSCENRGIISLSEISPPRFPIYHLLLSGNLLNRLYPNEFVNYTGASILHLGSNVIQDIETGAFHGLRGLRRLHLNNNKLELLRDDTFLGLENLEYLQVDYNYISVIEPNAFGKLHLLQVLILNDNLLSSLPNNLFRFVPLTHLDLRGNRLKLLPYVGLLQHMDKVVELQLEENPWNCSCELISLKDWLDSISYSALVGDVVCETPFRLHGRDLDEVSKQELCPRRLISDYEMRPQTPLSTTGYLHTTPASVNSVATSSSAVYKPPLKPPKGTRQPNKPRVRPTSRQPSKDLGYSNYGPSIAYQTKSPVPLECPTACSCNLQISDLGLNVNCQERKIESIAELQPKPYNPKKMYLTENYIAVVRRTDFLEATGLDLLHLGNNRISMIQDRAFGDLTNLRRLYLNGNRIERLSPELFYGLQSLQYLFLQYNLIREIQSGTFDPVPNLQLLFLNNNLLQAMPSGVFSGLTLLRLNLRSNHFTSLPVSGVLDQLKSLIQIDLHDNPWDCTCDIVGMKLWVEQLKVGVLVDEVICKAPKKFAETDMRSIKSELLCPDYSDVVVSTPTPSSIQVPARTSAVTPAVRLNSTGAPASLGAGGGASSVPLSVLILSLLLVFIMSVFVAAGLFVLVMKRRKKNQSDHTSTNNSDVSSFNMQYSVYGGGGGTGGHPHAHVHHRGPALPKVKTPAGHVYEYIPHPLGHMCKNPIYRSREGNSVEDYKDLHELKVTYSSNHHLQQQQQPPPPPQQPQQQPPPQLQLQPGEEERRESHHLRSPAYSVSTIEPREDLLSPVQDADRFYRGILEPDKHCSTTPAGNSLPEYPKFPCSPAAYTFSPNYDLRRPHQYLHPGAGDSRLREPVLYSPPSAVFVEPNRNEYLELKAKLNVEPDYLEVLEKQTTFSQF

SEQ ID NO: 33 - protein, 1428 aa, Mus musculus
MVAVAVAASTEARLRGSTTATAAPAGRKGRQHRPCTATGAWRPGPRARLCLPRVLSRALPPPPLLPLLFSLLLLPLPREAEAAAVAAAVSGSAAAEAKECDRPCVNGGRCNPGTGQCVCPTGWVGEQCQHCGGRFRLTGSSGFVTDGPGNYKYKTKCTWLIEGYPNAVLRLRFNHFATECSWDHLYVYDGDSIYAPLIAAFSGLIVPERDGNETAPEVTVTSGYALLHFFSDAAYNLTGFNITYNFDMCPNNCSARGECKSSNSSSAVECECSENWKGESCDIPHCTDNCGFPHRGICNASDTRGCSCFPHWQGPGCSIPVPANQSFWTREEYSDLKLPRASHKAVVNGNIMWVVGGYMFNHSDYSMVLAYDLTSREWLPLNHSVNSVVVRYGHSLALHKDKIYMYGGKIDSTGNVTNELRVFHIHNESWVLLTPKAKDQYAVVGHSAHIVTLASGRVVMLVIFGHCPLYGYISVVQEYDLEKNTWSILHTQGALVQGGYGHSSAYDDRTKALYVHGGYKAFSANKYRLADDLYRYDVDTQMWTILKDSRFFRYLHTAVIVSGTMLVFGGNTHNDTSMSHGAKCFSSDFMAYDIACDRWSVLPRPELHHDVNRFGHSAVLYNSTMYVFGGFNSLLLSDVLVFTSEQCDAHRSEAACVAAGPGIRCLWDTQSSRCTSWELATEEQAEKLKSECFSKRTLDHDRCDQHTDCYSCTANTNDCHWCNDHCVPVNHSCTEGQISIAKYESCPKDNPMYYCNKKTSCRSCALDQNCQWEPRNQECIALPENICGNGWHLVGNSCLKITTAKENYDNAKLSCRNHNAFLASLTSQKKVELVLKQLRLMQSSQSMSKLTLTPWVGLRKINVSYWCWEDMSPFTNSLLQWMPSEPSDAGFCGILSEPSTRGLKAATCINPLNGSVCERPANHSAKQCRTPCALRTACGECTSSSSECMWCSNMKQCVDSNAYVASFPFGQCMEWYTMSSCPPENCSGYCTCSHCLEQPGCGWCTDPSNTGKGKCIEGSYKGPVKMPSQASAGNVYPQPLLNSSMCLEDSRYNWSFIHCPACQCNGHSKCINQSVCEKCEDLTTGKHCETCISGFYGDPTNGGKCQPCKCNGHASLCNTNTGKCFCTTKGVKGDECQLCKVENRYQGNPLKGTCYYTLLIDYQFTFSLSQGDDRYYTAINFVATPDEQNRDFDMFINASKKFNLNITWATSFPAGTQTGEEVPVVSKTNIKEYKDSFSNEKFDFRNHPNITFFVYVSNFTWPIKIQIAFSQHSNFMDLVQFFVTFFSCFLSLLLVAAVVWKIKQSCWASRRREQLLREMQQMASRPFASVNVALETDEEPPDLIGGSIKTVPKPIALEPCFGNKAAVLSVFVRLPRGLGGIPPPGQSGLAVASALVDISQQMPIVYKEKSGAVRNRKQQPPAQPGTCI

SEQ ID NO: 34 - protein, 1428 aa, Mus musculus
MVAVAAAAATEARLRGSTTTTAAPAGRKGRQHRPCTATGAWRPGPRARLCLPRVLSRALPPPPLLPLLFSLLLLPLPREAEAAAVAAAVSGSAAAEAKECDRPCVNGGRCNPGTGQCVCPTGWVGEQCQHCGGRFRLTGSSGFVTDGPGNYKYKTKCTWLIEGQPNRIMRLRFNHFATECSWDHLYVYDGDSIYAPLIAAFSGLIVPERDGNETAPEVTVTSGYALLHFFSDAAYNLTGFNITYNFDMCPNNCSGRGECKSSNSSSAVECECSENWKGESCDIPHCTDNCGFPHRGICNASDTRGCSCFPHWQGPGCSIPVPANQSFWTREEYSDLKLPRASHKAVVNGNIMWVVGGYMFNHSDYSMVLAYDLTSREWLPLNHSVNSVVVRYGHSLALHKDKIYMYGGKIDSTGNVTNELRVFHIHNESWVLLTPKAKDQYAVVGHSAHIVTLASGRVVMLVIFGHCPLYGYISVVQEYDLEKNTWSILHTQGALVQGGYGHSSVYDDRTKALYVHGGYKAFSANKYRLADDLYRYDVDTQMWTILKDSRFFRYLHTAVIVSGTMLVFGGNTHNDTSMSHGAKCFSSDFMAYDIACDRWSVLPRPELHHDVNRFGHSAVLYNSTMYVFGGFNSLLLSDVLVFTSEQCDAHRSEAACVAAGPGIRCLWDTQSSRCTSWELATEEQAEKLKSECFSKRTLDHDRCDQHTDCYSCTANTNDCHWCNDHCVPVNHSCTEGQISIAKYESCPKDNPMYYCNKKTSCRSCALDQNCQWEPRNQECIALPENICGNGWHLVGNSCLKITTAKENYDNAKLSCRNHNAFLASLTSQKKVEFVLKQLRLMQSSQSMSKLTLTPWVGLRKINVSYWCWEDMSPFTNSLLQWMPSEPSDAGFCGILSEPSTRGLKAATCINPLNGSVCERPANHSAKQCRTPCALRTACGECTSSSSECMWCSNMKQCVDSNAYVASFPFGQCMEWYTMSSCPPENCSGYCTCSHCLEQPGCGWCTDPSNTGKGKCIEGSYKGPVKMPSQASAGNVYPQPLLNSSMCLEDSRYNWSFIHCPACQCNGHSKCINQSICEKCEDLTTGKHCETCISGFYGDPTNGGKCQPCKCNGHASLCNTNTGKCFCTTKGVKGDECQLCEVENRYQGNPLKGTCYYTLLIDYQFTFSLSQEDDRYYTAINFVATPDEQNRDLDMFINASKNFNLNITWATSFPAGTQTGEEVPVVSKTNIKEYKDSFSNEKFDFRNHPNITFFVYVSNFTWPIKIQIAFSQHSNFMDLVQFFVTFFSCFLSLLLVAAVVWKIKQSCWASRRREQLLREMQQMASRPFASVNVALETDEEPPDLIGGSIKTVPKPIALEPCFGNKAAVLSVFVRLPRGLGGIPPPGQSGLAVASALVDISQQMPIVYKEKSGAVRNRKQQPPAQPGTCI

SEQ ID NO: 35 - protein, 1198 aa, Homo sapiens
MVAAAAATEARLRRRTAATAALAGRSGGPHCVNGGRCNPGTGQCVCPAGWVGEQCQHCGGRFRLTGSSGFVTDGPGNYKYKTKCTWLIEGQPNRIMRLRFNHFATECSWDHLYVYDGDSIYAPLVAAFSGLIVPERDGNETVPEVVATSGYALLHFFSDAAYNLTGFNITYSFDMCPNNCSGRGECKISNSSDTVECECSENWKGEACDIPHCTDNCGFPHRGICNSSDVRGCSCFSDWQGPGCSVPVPANQSFWTREEYSNLKLPRASHKAVVNGNIMWVVGGYMFNHSDYNMVLAYDLASREWLPLNRSVNNVVVRYGHSLALYKDKIYMYGGKIDSTGNVTNELRVFHIHNESWVLLTPKAKEQYAVVGHSAHIVTLKNGRVVMLVIFGHCPLYGYISNVQEYDLDKNTWSILHTQGALVQGGYGHSSVYDHRTRALYVHGGYKAFSANKYRLADDLYRYDVDTQMWTILKDSRFFRYLHTAVIVSGTMLVFGGNTHNDTSMSHGAKCFSSDFMAYDIACDRWSVLPRPDSTMMSTDLAIPAVLHNSTMYVFGGFNSLLLSDILVFTSEQCDAHRSEAACLAAGPGIRCVWNTGSSQCISWALATDEQEEKLKSECFSKRTLDHDRCDQHTDCYSCTANTNDCHWCNDHCVPRNHSCSEGQISIFRYENCPKDNPMYYCNKKTSCRSCALDQNCQWEPRNQECIALPENICGIGWHLVGNSCLKITTAKENYDNAKLFCRNHNALLASLTTQKKVEFVLKQLRIMQSSQSMSKLTLTPWVGLRKINVSYWCWEDMSPFTNSLLQWMPSEPSDAGFCGILSEPSTRGLKAATCINPLNGSVCERPANHSAKQCRTPCALRTACGDCTSGSSECMWCSNMKQCVDSNAYVASFPFGQCMEWYTMSTCPPENCSGYCTCSHCLEQPGCGWCTDPSNTGKGKCIEGSYKGPVKMPSQAPTGNFYPQPLLNSSMCLEDSRYNWSFIHCPACQCNGHSKCINQSICEKCENLTTGKHCETCISGFYGDPTNGGKCQPCKCNGHASLCNTNTGKCFCTTKGVKGDECQLCEVENRYQGNPLRGTCYYTLLIDYQFTFSLSQEDDRYYTAINFVATPDEQNRDLDMFINASKNFNLNITWAASFSAGTQAGEEMPVVSKTNIKEYKDSFSNEKFDFRNHPNITFFVYVSNFTWPIKIQVQTEQ

SEQ ID NO: 36 - protein, 1275 aa, Rattus norvegicus
MVAAAAAAEATEARLRGYTTATAAPAGWKERQHRPCAATGAWRPWPRAGLCLPRVLSRALSPPPLLPLLPLLFSLLLLPLPREAEAAAVAAAVSGSAAAEAKECDRPCVNGGRCNPGTGQCVCPTGWVGEQCQHCGGRFRLTGSSGFVTDGPGNYKYKTKCTWLIEGQPNKIMRLRFNHFATECSWDHLYVYDGDSIYAPLIAAFSGLIVPERDGNETAPEVTVTSGYALLHFFSDAAYNLTGFNITYNFDMCPNNCSGRGECKSSNSSSTVECECSENWKGESCDIPHCTDNCGFPHRGICNASDTRGCSCFPHWQGPGCSIPVPANQSFWTREEYSDLKLPRASHKAEVNGNIMWVVGGYMFNHSDYSMVLAYDLASREWLSLNHSVNSVVVRYGHSLALHKDKIYMYGGKIDSTGNVTNELRVFHIHNESWVLLTPKAKDQYAVVGHSAHIVTLSSGRVVMLVIFGHCPLYGYISVVQEYDLEKNTWSILQTQGALVQGGYGHSSVYDHRTKALYVHGGYKAFSANKYRLADDLYRYHVDTQMWTILKDSRFFRYLHTAVIVSGTMLVFGGNTHNDTSMSHGAKCFSSDFMAYDIACDRWSVLPRPELHHDVNRFGHSAVLHNSTMYVFGGFNSLLLSDVLVFTSEQCDAHRSEAACVAAGPGIRCLWDTQSSRCTSWELATEEQAEKLKSECFSKRTLDHDRCDQHTDCYSCTANTNDCHWCNDHCVPVNHSCTEGQISIAKYDNCPKDNPMYYCNKKTSCRSCALDQNCQWEPRNQECIALPENICGIGWHLVGNSCLKITTAKENYDNAKLSCRNHNAFLASLTSQKKVEFVLKQLRLMQSSQSTSKLTLTPWVGLRKINVSYWCWEDMSPFTNSLLQWMPSEPSDAGFCGILSEPSTRGLKAATCINPLNGSVCERPANHSAKQCRTPCALRTACGECTSSSSECMWCSNMKQCVDSNAYVASFPFGQCMEWYTMSSCPPENCSGYCTCSHCLEQPGCGWCTDPSNTGKGKCIEGSYKGPVKMPSHASTGNVYPQPLLNSSMCLEDSRYNWSFIHCPACQCNGHSKCINQSICEKCEDLTTGKHCETCISGFYGDPTNGGKCQPCKCNGHASLCNTNTGKCFCTTKGVKGEECQLCEVENRYQGNPLKGTCYYTLLIDYQFTFSLSQEDDRYYTAINFVATPDEQNRDLDMFINASKNFNLNITWATSFPAGTQTGEEVPVVSKTNIKEYKDSFSNEKFDFRNHPNITFFVYVSNFTWPIKIQVRVTS

SEQ ID NO: 37 - protein, 883 aa, Homo sapiens
MLEMNARSLQQKLETERELKQRLLEEQAKLQQQMDLQKNHIFRLTQGLQEALDRADLLKTERSDLEYQLENIQVLYSHEKVKMEGTISQQTKLIDFLQAKMDQPAKKKKVPLQYNELKLALEKEKARCAELEEALQKTRIELRSAREEAAHRKATDHPHPSTPATARQQIAMSAIVRSPEHQPSAMSLLAPPSSRRKESSTPEEFSRRLKERMHHNIPHRFNVGLNMRATKCAVCLDTVHFGRQASKCLECQVMCHPKCSTCLPATCGLPAEYATHFTEAFCRDKMNSPGLQTKEPSSSLHLEGWMKVPRNNKRGQQGWDRKYIVLEGSKVLIYDNEAREAGQRPVEEFELCLPDGDVSIHGAVGASELANTAKADVPYILKMESHPHTTCWPGRTLYLLAPSFPDKQRWVTALESVVAGGRVSREKAEADAKLLGNSLLKLEGDDRLDMNCTLPFSDQVVLVGTEEGLYALNVLKNSLTHVPGIGAVFQIYIIKDLEKLLMIAGEERALCLVDVKKVKQSLAQSHLPAQPDISPNIFEAVKGCHLFGAGKIENGLCICAAMPSKVVILRYNENLSKYCIRKEIETSEPCSCIHFTNYSILIGTNKFYEIDMKQYTLEEFLDKNDHSLAPAVFAASSNSFPVSIVQVNSAGQREEYLLCFHEFGVFVDSYGRRSRTDDLKWSRLPLAFAYREPYLFVTHFNSLEVIEIQARSSAGTPARAYLDIPNPRYLGPAISSGAIYLASSYQDKLRVICCKGNLVKESGTEHHRGPSTSRSSPNKRGPPTYNEHITKRVASSPAPPEGPSHPREPSTPHRYREGRTELRRDKSPGRPLEREKSPGRMLSTRRERSPGRLFEDSSRGRLPAGAVRTPLSQVNKVWDQSSV

SEQ ID NO: 38 - protein, 1286 aa, Homo sapiens
VLDNQIKKDLADKETLENMMQRHEEEAHEKGKILSEQKAMINAMDSKIRSLEQRIVELSEANKLAANSSLFTQRNMKAQEEMISELRQQKFYLETQAGKLEAQNRKLEEQLEKISHQDHSDKNRLLELETRLREVSLEHEEQKLELKRQLTELQLSLQERESQLTALQAARAALESQLRQAKTELEETTAEAEEEIQALTAHRDEIQRKFDALRNSCTVITDLEEQLNQLTEDNAELNNQNFYLSKQLDEASGANDEIVQLRSEVDHLRREITEREMQLTSQKQTMEALKTTCTMLEEQVMDLEALNDELLEKERQWEAWRSVLGDEKSQFECRVRELQRMLDTEKQSRARADQRITESRQVVELAVKEHKAEILALQQALKEQKLKAESLSDKLNDLEKKHAMLEMNARSLQQKLETERELKQRLLEEQAKLQQQMDLQKNHIFRLTQGLQEALDRADLLKTERSDLEYQLENIQVLYSHEKVKMEGTISQQTKLIDFLQAKMDQPAKKKKVPLQYNELKLALEKEKARCAELEEALQKTRIELRSAREEAAHRKATDHPHPSTPATARQQIAMSAIVRSPEHQPSAMSLLAPPSSRRKESSTPEEFSRRLKERMHHNIPHRFNVGLNMRATKCAVCLDTVHFGRQASKCLECQVMCHPKCSTCLPATCGLPAEYATHFTEAFCRDKMNSPGLQTKEPSSSLHLEGWMKVPRNNKRGQQGWDRKYIVLEGSKVLIYDNEAREAGQRPVEEFELCLPDGDVSIHGAVGASELANTAKADVPYILKMESHPHTTCWPGRTLYLLAPSFPDKQRWVTALESVVAGGRVSREKAEADAKLLGNSLLKLEGDDRLDMNCTLPFSDQVVLVGTEEGLYALNVLKNSLTHVPGIGAVFQIYIIKDLEKLLMIAGEERALCLVDVKKVKQSLAQSHLPAQPDISPNIFEAVKGCHLFGAGKIENGLCICAAMPSKVVILRYNENLSKYCIRKEIETSEPCSCIHFTNYSILIGTNKFYEIDMKQYTLEEFLDKNDHSLAPAVFAASSNSFPVSIVQVNSAGQREEYLLCFHEFGVFVDSYGRRSRTDDLKWSRLPLAFAYREPYLFVTHFNSLEVIEIQARSSAGTPARAYLDIPNPRYLGPAISSGAIYLASSYQDKLRVICCKGNLVKESGTEHHRGPSTSRSSPNKRGPPTYNEHITKRVASSPAPPEGPSHPREPSTPHRYREGRTELRRDKSPGRPLEREKSPGRMLSTRRERSPGRLFEDSSRGRLPAGAVRTPLSQVNKVWDQSSV

SEQ ID NO: 39 - protein, 940 aa, Homo sapiens
QSRARADQRITESRQVVELAVKEHKAEILALQQALKEQKLKAESLSDKLNDLEKKHAMLEMNARSLQQKLETERELKQRLLEEQAKLQQQMDLQKNHIFRLTQGLQEALDRADLLKTERSDLEYQLENIQVLYSHEKVKMEGTISQQTKLIDFLQAKMDQPAKKKKVPLQYNELKLALEKEKARCAELEEALQKTRIELRSAREEAAHRKATDHPHPSTPATARQQIAMSAIVRSPEHQPSAMSLLAPPSSRRKESSTPEEFSRRLKERMHHNIPHRFNVGLNMRATKCAVCLDTVHFGRQASKCLECQVMCHPKCSTCLPATCGLPAEYATHFTEAFCRDKMNSPGLQTKEPSSSLHLEGWMKVPRNNKRGQQGWDRKYIVLEGSKVLIYDNEAREAGQRPVEEFELCLPDGDVSIHGAVGASELANTAKADVPYILKMESHPHTTCWPGRTLYLLAPSFPDKQRWVTALESVVAGGRVSREKAEADAKLLGNSLLKLEGDDRLDMNCTLPFSDQVVLVGTEEGLYALNVLKNSLTHVPGIGAVFQIYIIKDLEKLLMIAGEERALCLVDVKKVKQSLAQSHLPAQPDISPNIFEAVKGCHLFGAGKIENGLCICAAMPSKVVILRYNENLSKYCIRKEIETSEPCSCIHFTNYSILIGTNKFYEIDMKQYTLEEFLDKNDHSLAPAVFAASSNSFPVSIVQVNSAGQREEYLLCFHEFGVFVDSYGRRSRTDDLKWSRLPLAFAYREPYLFVTHFNSLEVIEIQARSSAGTPARAYLDIPNPRYLGPAISSGAIYLASSYQDKLRVICCKGNLVKESGTEHHRGPSTSRSSPNKRGPPTYNEHITKRVASSPAPPEGPSHPREPSTPHRYREGRTELRRDKSPGRPLEREKSPGRMLSTRRERSPGRLFEDSSRGRLPAGAVRTPLSQVNKVWDQSSV

SEQ ID NO: 40 - protein, 1641 aa, Mus musculus
PFVPTLKSDDDTSNFDEPEKNSWVSSSVCQLSPSGFSGEELPFVGFSYSKALGYLGRSESVVSSLDSPAKVSSMEKKLLIKSKELQDSQDKCHKMEQEMTRLHRRVSEVEAVLSQKEVELKASETQRSLLEQDLATYITECSSLKRSLEQARMEVSQEDDKALQLLHDIREQSRKLQEIKEQEYQAQVEEMRLMMNQLEEDLVSARRRSDLYESELRESRLAAEEFKRKANECQHKLMKAKDQGKPEVGEYSKLEKINAEQQLKIQELQEKLEKAVKASTEATELLQNIRQAKERAERELEKLHNREDSSEGIKKKLVEAEELEEKHREAQVSAQHLEVHLKQKEQHYEEKIKVLDNQIKKDLADKESLENMMQRHEEEAHEKGKILSEQKAMINAMDSKIRSLEQRIVELSEANKLAANSSLFTQRNMKAQEEMISELRQQKFYLETQAGKLEAQNRKLEEQLEKISHQDHSDKSRLLELETRLREVSLEHEEQKLELKRQLTELQLSLQERESQLTALQAARAALESQLRQAKTELEETTAEAEEEIQALTAHRDEIQRKFDALRNSCTVITDLEEQLNQLTEDNAELNNQNFYLSKQLDEASGANDEIVQLRSEVDHLRREITEREMQLTSQKQTMEALKTTCTMLEEQVLDLEALNDELLEKERQWEAWRSVLGDEKSQFECRVRELQRMLDTEKQSRARADQRITESRQVVELAVKEHKAEILALQQALKEQKLKAESLSDKLNDLEKKHAMLEMNARSLQQKLETERELKQRLLEEQAKLQQQMDLQKNHIFRLTQGLQEALDRADLLKTERSDLEYQLENIQVLYSHEKVKMEGTISQQTKLIDFLQAKMDQPAKKKKVPLQYNELKLALEKEKARCAELEEALQKTRIELRSAREEAAHRKATDHPHPSTPATARQQIAMSAIVRSPEHQPSAMSLLAPPSSRRKESSTPEEFSRRLKERMHHNIPHRFNVGLNMRATKCAVCLDTVHFGRQASKCLECQVMCHPKCSTCLPATCGLPAEYATHFTEAFCRDKMNSPGLQSKEPGSSLHLEGWMKVPRNNKRGQQGWDRKYIVLEGSKVLIYDNEAREAGQRPVEEFELCLPDGDVSIHGAVGASELANTAKADVPYILKMESHPHTTCWPGRTLYLLAPSFPDKQRWVTALESVVAGGRVSREKAEADAKLLGNSLLKLEGDDRLDMNCTLPFSDQVVLVGTEEGLYALNVLKNSLTHIPGIGAVFQIYIIKDLEKLLMIAGEERALCLVDVKKVKQSLAQSHLPAQPDVSPNIFEAVKGCHLFAAGKIENSLCICAAMPSKVVILRYNDNLSKYCIRKEIETSEPCSCIHFTNYSILIGTNKFYEIDMKQYTLDEFLDKNDHSLAPAVFASSSNSFPVSIVQANSAGQREEYLLCFHEFGVFVDSYGRRSRTDDLKWSRLPLAFAYREPYLFVTHFNSLEVIEIQARSSLGSPARAYLEIPNPRYLGPAISSGAIYLASSYQDKLRVICCKGNLVKESGTEQHRVPSTSRSSPNKRGPPTYNEHITKRVASSPAPPEGPSHPREPSTPHRYRDREGRTELRRDKSPGRPLEREKSPGRMLSTRRERSPGRLFEDSSRGRLPAGAVRTPLSQVNKVWDQSSV

SEQ ID NO: 41 - protein, 1597 aa, Mus musculus
MLLGEEAMMEQEMTRLHRRVSEVEAVLSQKEVELKASETQRSLLEQDLATYITECSSLKRSLEQARMEVSQEDDKALQLLHDIREQSRKLQEIKEQEYQAQVEEMRLMMNQLEEDLVSARRRSDLYESELRESRLAAEEFKRKANECQHKLMKAKDQGKPEVGEYSKLEKINAEQQLKIQELQEKLEKAVKASTEATELLQNIRQAKERAERELEKLHNREDSSEGIKKKLVEAEERRHSLENKVKRLETMERRENRLKDDIQTKSEQIQQMADKILELEEKHREAQVSAQHLEVHLKQKEQHYEEKIKVLDNQIKKDLADKESLENMMQRHEEEAHEKGKILSEQKAMINAMDSKIRSLEQRIVELSEANKLAANSSLFTQRNMKAQEEMISELRQQKFYLETQAGKLEAQNRKLEEQLEKISHQDHSDKSRLLELETRLREVSLEHEEQKLELKRQLTELQLSLQERESQLTALQAARAALESQLRQAKTELEETTAEAEEEIQALTAHRDEIQRKFDALRNSCTVITDLEEQLNQLTEDNAELNNQNFYLSKQLDEASGANDEIVQLRSEVDHLRREITEREMQLTSQKQTMEALKTTCTMLEEQVLDLEALNDELLEKERQWEAWRSVLGDEKSQFECRVRELQRMLDTEKQSRARADQRITESRQVVELAVKEHKAEILALQQALKEQKLKAESLSDKLNDLEKKHAMLEMNARSLQQKLETERELKQRLLEEQAKLQQQMDLQKNHIFRLTQGLQEALDRADLLKTERSDLEYQLENIQVLYSHEKVKMEGTISQQTKLIDFLQAKMDQPAKKKKVPLQYNELKLALEKEKARCAELEEALQKTRIELRSAREEAAHRKATDHPHPSTPATARQQIAMSAIVRSPEHQPSAMSLLAPPSSRRKESSTPEEFSRRLKERMHHNIPHRFNVGLNMRATKCAVCLDTVHFGRQASKCLECQVMCHPKCSTCLPATCGLPAEYATHFTEAFCRDKMNSPGLQSKEPGSSLHLEGWMKVPRNNKRGQQGWDRKYIVLEGSKVLIYDNEAREAGQRPVEEFELCLPDGDVSIHGAVGASELANTAKADVPYILKMESHPHTTCWPGRTLYLLAPSFPDKQRWVTALESVVAGGRVSREKAEADAKLLGNSLLKLEGDDRLDMNCTLPFSDQVVLVGTEEGLYALNVLKNSLTHIPGIGAVFQIYIIKDLEKLLMIAGEERALCLVDVKKVKQSLAQSHLPAQPDVSPNIFEAVKGCHLFAAGKIENSLCICAAMPSKVVILRYNDNLSKYCIRKEIETSEPCSCIHFTNYSILIGTNKFYEIDMKQYTLDEFLDKNDHSLAPAVFASSSNSFPVSIVQANSAGQREEYLLCFHEFGVFVDSYGRRSRTDDLKWSRLPLAFAYREPYLFVTHFNSLEVIEIQARSSLGSPARAYLEIPNPRYLGPAISSGAIYLASSYQDKLRVICCKGNLVKESGTEQHRVPSTSRSSPNKRGPPTYNEHITKRVASSPAPPEGPSHPREPSTPHRYRDREGRTELRRDKSPGRPLEREKSPGRMLSTRRERSPGRLFEDSSRGRLPAGAVRTPLSQVNKVWDQSSV

SEQ ID NO: 42 - protein, 1871 aa, Homo sapiens
MPSVCLLLLLFLAVGGALGNRPFRAFVVTDTTLTHLAVHRVTGEVFVGAVNRVFKLAPNLTELRAHVTGPVEDNARCYPPPSMRVCAHRLAPVDNINKLLLIDYAARRLVACGSIWQGICQFLRLDDLFKLGEPHHRKEHYLSGAQEPDSMAGVIVEQGQGPSKLFVGTAVDGKSEYFPTLSSRKLISDEDSADMFSLVYQDEFVSSQIKIPSDTLSLYPAFDIYYIYGFVSASFVYFLTLQLDTQQTLLDTAGEKFFTSKIVRMCAGDSEFYSYVEFPIGCSWRGVEYRLVQSAHLAKPGLLLAQALGVPADEDVLFTIFSQGQKNRASPPRQTILCLFTLSNINAHIRRRIQSCYRGEGTLALPWLLNKELPCINTPMQINGNFCGLVLNQPLGGLHVIEGLPLLADSTDGMASVAAYTYRQHSVVFIGTRSGSLKKVRVDGFQDAHLYETVPVVDGSPILRDLLFSPDHRHIYLLSEKQVSQLPVETCEQYQSCAACLGSGDPHCGWCVLRHRCCREGACLGASAPHGFAEELSKCVQVRVRPNNVSVTSPGVQLTVTLHNVPDLSAGVSCAFEAAAENEAVLLPSGELLCPSPSLQELRALTRGHGATRTVRLQLLSKETGVRFAGADFVFYNCSVLQSCMSCVGSPYPCHWCKYRHTCTSRPHECSFQEGRVHSPEGCPEILPSGDLLIPVGVMQPLTLRAKNLPQPQSGQKNYECVVRVQGRQQRVPAVRFNSSSVQCQNASYSYEGDEHGDTELDFSVVWDGDFPIDKPPSFRALLYKCWAQRPSCGLCLKADPRFNCGWCISEHRCQLRTHCPAPKTNWMHLSQKGTRCSHPRITQIHPLVGPKEGGTRVTIVGDNLGLLSREVGLRVAGVRCNSIPAEYISAERIVCEMEESLVPSPPPGPVELCVGDCSADFRTQSEQVYSFVTPTFDQVSPSRGPASGGTRLTISGSSLDAGSRVTVTVRDSECQFVRRDAKAIVCISPLSTLGPSQAPITLAIDRANISSPGLIYTYTQDPTVTRLEPTWSIINGSTAITVSGTHLLTVQEPRVRAKYRGIETTNTCQVINDTAMLCKAPGIFLGRPQPRAQGEHPDEFGFLLDHVQTARSLNRSSFTYYPDPSFEPLGPSGVLDVKPGSHVVLKGKNLIPAAAGSSRLNYTVLIGGQPCSLTVSDTQLLCDSPSQTGRQPVMVLVGGLEFWLGTLHISAERALTLPAMMGLAAGGGLLLLAITAVLVAYKRKTQDADRTLKRLQLQMDNLESRVALECKEAFAELQTDINELTNHMDEVQIPFLDYRTYAVRVLFPGIEAHPVLKELDTPPNVEKALRLFGQLLHSRAFVLTFIHTLEAQSSFSMRDRGTVASLTMVALQSRLDYATGLLKQLLADLIEKNLESKNHPKLLLRRTESVAEKMLTNWFTFLLHKFLKECAGEPLFLLYCAIKQQMEKGPIDAITGEARYSLSEDKLIRQQIDYKTLTLHCVCPENEGSAQVPVKVLNCDSITQAKDKLLDTVYKGIPYSQRPKAEDMDLEWRQGRMTRIILQDEDVTTKIECDWKRLNSLAHYQVTDGSLVALVPKQVSAYNMANSFTFTRSLSRYESLLRTASSPDSLRSRAPMITPDQETGTKLWHLVKNHDHADHREGDRGSKMVSEIYLTRLLATKGTLQKFVDDLFETVFSTAHRGSALPLAIKYMFDFLDEQADQRQISDPDVRHTWKSNCLPLRFWVNVIKNPQFVFDIHKNSITDACLSVVAQTFMDSCSTSEHRLGKDSPSNKLLYAKDIPNYKSWVERYYRDIAKMASISDQDMDAYLVEQSRLHASDFSVLSALNELYFYVTKYRQEILTALDRDASCRKHKLRQKLEQIISLVSSDS

SEQ ID NO: 43 - protein, 1963 aa, Homo sapiens
GFGRLPDSELRAGRGASRRPQQPAAAEVDRAGTEGQTDVAELESCEGQPGKVEQMSTHRSRLLTAAPLSMEQRRPWPRALEVDSRSVVLLSVVWVLLAPPAAGMPQFSTFHSENRDWTFNHLTVHQGTGAVYVGAINRVYKLTGNLTIQVAHKTGPEEDNKSCYPPLIVQPCSEVLTLTNNVNKLLIIDYSENRLLACGSLYQGVCKLLRLDDLFILVEPSHKKEHYLSSVNKTGTMYGVIVRSEGEDGKLFIGTAVDGKQDYFPTLSSRKLPRDPESSAMLDYELHSDFVSSLIKIPSDTLALVSHFDIFYIYGFASGGFVYFLTVQPETPEGVAINSAGDLFYTSRIVRLCKDDPKFHSYVSLPFGCTRAGVEYRLLQAAYLAKPGDSLAQAFNITSQDDVLFAIFSKGQKQYHHPPDDSALCAFPIRAINLQIKGRLQSCYQGEGNLELNWLLGKDVQCTKAPVPIDDNFCGLDINQPLGGSTPVEGLTLYTTSRDRMTSVASYVYNGYSVVFVGTKSGKLKKIRADGPPHGGVQYEMVSVLKDGSPILRDMAFSIDQRYLYVMSERQVTRVPVESCEQYTTCGECLSSGDPHCGWCALHNMCSRRDKCQQAWEPNRFAASISQCVSLAVHPSSISVSEHSRLLSLVVSDAPDLSAGIACAFGNLTEVEGQVSGSQVICISPGPKDVPVIPLDQDWFGLELQLRSKETGKIFVSTEFKFYNCSAHQLCLSCVNSAFRCHWCKYRNLCTHDPTTCSFQEGRINISEDCPQLVPTEEILIPVGEVKPITLKARNLPQPQSGQRGYECVLNIQGAIHRVPALRFNSSSVQCQNSSYQYDGMDISNLAVDFAVVWNGNFIIDNPQDLKVHLYKCAAQRESCGLCLKADRKFECGWCSGERRCTLHQHCTSPSSPWLDWSSHNVKCSNPQITEILTVSGPPEGGTRVTIHGVNLGLDFSEIAHHVQVAGVPCTPLPGEYIIAEQIVCEMGHALVGTTSGPVRLCIGECKPEFMTKSHQQYTFVNPSVLSLNPIRGPESGGTMVTITGHYLGAGSSVAVYLGNQTCEFYGRSMSEIVCVSPPSSNGLGPVPVSVSVDRAHVDSNLQFEYIDDPRVQRIEPEWSIASGHTPLTITGFNLDVIQEPRIRVKFNGKESVNVCKVVNTTTLTCLAPSLTTDYRPGLDTVERPDEFGFVFNNVQSLLIYNDTKFIYYPNPTFELLSPTGVLDQKPGSPIILKGKNLCPPASGGAKLNYTVLIGETPCAVTVSETQLLCEPPNLTGQHKVMVHVGGMVFSPGSVSVISDSLLTLPAIVSIAAGGSLLLIIVIIVLIAYKRKSRENDLTLKRLQMQMDNLESRVALECKEAFAELQTDINELTSDLDRSGIPYLDYRTYAMRVLFPGIEDHPVLRELEVQGNGQQHVEKALKLFAQLINNKVFLLTFIRTLELQRSFSMRDRGNVASLIMTGLQGRLEYATDVLKQLLSDLIDKNLENKNHPKLLLRRTESVAEKMLTNWFAFLLHKFLKECAGEPLFMLYCAIKQQMEKGPIDAITGEARYSLSEDKLIRQQIEYKTLILNCVNPDNENSPEIPVKVLNCDTITQVKEKILDAVYKNVPYSQRPRAVDMDLEWRQGRIARVVLQDEDITTKIEGDWKRLNTLMHYQVSDRSVVALVPKQTSSYNIPASASISRTSISRYDSSFRYTGSPDSLRSRAPMITPDLESGVKVWHLVKNHDHGDQKEGDRGSKMVSEIYLTRLLATKGTLQKFVDDLFETLFSTVHRGSALPLAIKYMFDFLDEQADRHSIHDTDVRHTWKSNCLPLRFWVNVIKNPQFVFDIHKGSITDACLSVVAQTFMDSCSTSEHRLGKDSPSNKLLYAKDIPSYKSWVERYYADIAKLPAISDQDMNAYLAEQSRLHAVEFNMLSALNEIYSYVSKYSEELIGALEQDEQARRQRLAYKVEQLINAMSIES

SEQ ID NO: 44 - protein, 1905 aa, Xenopus laevis
MLLHAERPLPFHLWTFLVLLGSWIATGDGSPKDFRTFTGSDWSLTHLVVHNKTGEVYVGAINRIYKLSNNLTLLRTHVTGPVEDNEKCYPPPSVQSCPHGLITTNNVNKLLLIDYSDNRLIACGSASQGICQFLRLDDLFKLGEPHHRKEHYLSSVNESGTMSGVIIEVPNGQNKLFVGTPIDGKSEYFPTLSSRKLLGNEENAEMFGFVYQDEFVSSQLKIPSDTLSKFPTFDIYYVYSFSSEQFVYYLTLQLDTQLTSPDSTGEQFFTSKIVRLCVDDPKFYSYVEFPIGCMKDGVEYRLIQDAYLSKPGKRLAKELGISEREDILFTVFSQGQKNRIKPPKESVLCLFTLKKIKDKIKERIQSCYRGDGKLSLPWLLNKELGCINSPLQIDDNFCGQDFNQPLGGTVTIEGTPLFLDKEDGMTSVAAYDYRGHTVVFAGTRSGRVKKILVDLSASSSHLVQQYENVVVHEGNAILRDLVLSPDRQYIYAMTEKQVTRVPVESCEQYESCDTCLGSRDPHCGWCVLHNMCSRKDKCERADELHRFTSDQRQCVQLTVHPKNISVTVSEVPMVLQAWNVPDLSAGVNCSFEDFTEMEGRILDGKIYCTSPSAKEVIPITRGHGDKRVVKLYLKSKETGKKFASVDFVFYNCSVHQSCLSCVNGSFPCHWCKYRHVCTHNAADCSFQEGRVNMSEDCPQILPSSQIYIPVGVVKPITLTAKNLPQPQSGQRNYECIFHIPGSVTRVTALRFNSTSIQCQNTSYNYEGNDISDLPVNLSVVWNGHFVIDNPQNIQAHLYKCSALRESCGLCLKSDRRFECGWCVSEKKCTLRQNCPTLENPWMHASTANSRCTDPKITKLFPETGPRQGGTRLTITGENLGLRFEDIRFGVRVGHVMCVPVESEYISAEQIVCEINDAGRTRVHEAQVEVCVKDCSQDYRAISPKSFTFVLPSFNRVTPSRGPLSGGTWISIEGNYLNAGSDVSVAIGGRPCMFSWRTAKEIRCKTPQGPSTGKAEIQILINRATMNNSEVHYNYTEDPTVQKIEPEWSIASGGTPLIVTGMNLATIKEPKIRAKYGDVEKENNCTLYNDTTMVCLAPSVDNPLRSPPENGDRPDEIGFIMDNVHALLIVNTTSFLYYPDPVFEPLTASGNLELKPSSPLIIKGRNLIPAAPGNFRLNYTVLIGDTPCALTVSETQLLCESPNLTGQHKVTIKAGGFEYSPGTLQIYSDSLLTLPAIIGIGGGGGLLLLIIIIVLIAYKRKSRDADRTLKRLQLQMDNLESRVALECKEAFAELQTDIHELTNDLDGAGIPFLEYRTYAMRVLFPGIEDHPVLKEMEVQANVEKSLTLFGQLLTKKHFLLTFIRTLEAQRSFSMRDRGNVASLIMTALQGEMEYATGVLKQLLSDLIEKNLESKNHPKLLLRRTESVAEKMLTNWFTFLLYKFLKECAGEPLFMLHCAIKQQMEKGPIDAITGEARYSLSEDKLIRQQIDYKTLNPCADDVGLSDESCCRSPQTLNCVNPENENAPEIPVKVLNCDTITQVKEKLLDAVYKGVPYSQRPKAGDMDLEWRQGRMARIILQDEDVTTKIDNDWKRLNTLAHYQVTDGSSVALVPKQNSAYNISNSSTFTKSLSRYESMLRTASSPDSLRSRTPMITPDLESGTKLWHLVKNHDHLDQREGDRGSKMVSEIYLTRLLATKGTLQKFVDDLFETIFSTAHRGSALPLAIKYMFDFLDEQADKHQITDYDVRHTWKSNCLPLRFWVNVIKNPQFVFDIHKNSITDACLSVVAQTFMDSCSTSEHKLGKDSPSNKLLYAKDIPNYKSWVERYYADIAKMPVISDQDMSAYLAEQSRLHLSQFNSMSALHEIYSYITKYRDEILTALEKDEQARRQRLRSKLEQVIDTMAQSS

SEQ ID NO: 45 - protein, 813 aa, Homo sapiens
GTRVVCKVVNTTTLTCLAPSLTTDYRPGLDTVERPDEFGFVFNNVQSLLIYNDTKFIYYPNPTFELLSPTGVLDQKPGSPIILKGKNLCPPASGGAKLNYTVLIGETPCAVTVSETQLLCEPPNLTGQHKVMVHVGGMVFSPGSVSVISDSLLTLPAIVSIAAGGSLLLIIVIIVLIAYKRKSRENDLTLKRLQMQMDNLESRVALECKEAFAELQTDINELTSDLDRSGIPYLDYRTYAMRVLFPGIEDHPVLRELEVQGNGQQHVEKALKLFAQLINNKVFLLTFIRTLELQRSFSMRDRGNVASLIMTGLQGRLEYATDVLKQLLSDLIDKNLENKNHPKLLLRRTESVAEKMLTNWFAFLLHKFLKECAGEPLFMLYCAIKQQMEKGPIDAITGEARYSLSEDKLIRQQIEYKTLILNCVNPDNENSPEIPVKVLNCDTITQVKEKILDAVYKNVPYSQRPRAVDMDLEWRQGRIARVVLQDEDITTKIEGDWKRLNTLMHYQVSDRSVVALVPKQTSSYNIPASASISRTSISRYDSSFRYTGSPDSLRSRAPMITPDLESGVKVWHLVKNHDHGDQKEGDRGSKMVSEIYLTRLLATKGTLQKFVDDLFETLLSTVHRGSALPLAIKYMFDFLDEQADRHSIHDTDVRHTWKSNCLPLRFWVNVIKNPQFVFDIHKGSITDACLSVVAQTFMDSCSTSEHRLGKDSPSNKLLYAKDIPSYKSWVERYYADIAKLPAISDQDMNAYLAEQSRLHAVEFNMLSALNEIYSYVSKYSEELIGALEQDEQARRQRLAYKVEQLINAMSIES

SEQ ID NO: 46 - protein, 593 aa, Homo sapiens
VELTVVWNGHFNIDNPAQNKVHLYKCGAMRESCGLCLKADPDFACGWCQGPGQCTLRQHCPAQESQWLELSGAKSKCTNPRITEIIPVTGPREGGTKVTIRGENLGLEFRDIASHVKVAGVECSPLVDGYIPAEQIVCEMGEAKPSQHAGFVEICVAVCRPEFMARSSQLYYFMTLTLSDLKPSRGPMSGGTQVTITGTNLNAGSNVVVMFGKQPCLFHRRSPSYIVCNTTSSDEVLEMKVSVQVDRAKIHQDLVFQYVEDPTIVRIEPEWSIVSGNTPIAVWGTHLDLIQNPQIRAKHGGKEHINICEVLNATEMTCQAPALALGPDHQSDLTERPEEFGFILDNVQSLLILNKTNFTYYPNPVFEAFGPSGILELKPGTPIILKGKNLIPPVAGGNVKLNYTVLVGEKPCTVTVSDVQLLCESPNLIGRHKVMARVGGMEYSPGMVYIAPDSPLSLPAIVSIAVAGGLLIIFIVAVLIAYKRKSRESDLTLKRLQMQMDNLESRVALECKEGTEWPHAGGHVCVRVCICVCMHICVCVCICFIYKQAGWAAVGSAGGWRCVCLCECVCVHVCVCTSVCIYVSYTSKQAGQQ

SEQ ID NO: 47 - protein, 477 aa, Homo sapiens
MLPPGSNGTAYPGQFALYQQLAQGNAVGGSAGAPPLGPSQVVTACLLTLLIIWTLLGNVLVCAAIVRSRHLRANMTNVFIVSLAVSDLFVALLVMPWKAVAEVAGYWPFGAFCDVWVAFDIMCSTASILNLCVISVDRYWAISRPFRYKRKMTQRMALVMVGLAWTLSILISFIPVQLNWHRDQAASWGGLDLPNNLANWTPWEEDFWEPDVNAENCDSSLNRTYAISSSLISFYIPVAIMIVTYTRIYRIAQVQIRRISSLERAAEHAQSCRSSAACAPDTSLRASIKKETKVLKTLSVIMGVFVCCWLPFFILNCMVPFCSGHPEGPPAGFPCVSETTFDVFVWFGWANSSLNPVIYAFNADFQKVFAQLLGCSHFCSRTPVETVNISNELISYNQDIVFHKEIAAAYIHMMPNAVTPGNREVDNDEEEGPFDRMFQIYQTSPDGDPVAESVWELDCEGEISLDKITPFTPNGFH

SEQ ID NO: 48 - protein, 475 aa, Homo sapiens [sequence omitted]

SEQ ID NO: 49 - protein, 457 aa, Homo sapiens
MYQPFQHLDSDQVASWQSPEMLMNKSVSRESQRRKELVAGQIVTGSLLLLLIFWTLFGNILVCTAVMRFRHLRSRVTNIFIVSLAVSDLLVALLVMPWKAVAEVAGHWPFGAFCDIWVAFDIMCSTASILNLCVISVDRYWAISSPFRYERKMTQRVALLMISTAWALSVLISFIPVQLSWHKSETEDHLLSNHSTGNCDSSLNRTYAISSSLISFYIPVAIMIVTYTRIYRIAQIQIKRISTLERAAEHAQSCRSNRVDSCSRHHQTSLRTSIKKETKVLKTLSIIMGVFVCCWLPFFILNCMVPFCDRSPGHPQAGLPCVSETTFDIFVWFGWANSSLNPIIYAFNADFRKVFSSLLGCGHWCSTTPVETVNISNELISYNQDTLFHKDIVTAYVNMIPNVVDCIDDNEDAFDHMSQISQTSANNELATDSMCELDSEVDISLHKITPSMSNGIH

SEQ ID NO: 50 - protein, 486 aa, Gallus gallus
MLRGGRSPLPPPAGPPGGARGQAGAGAAQVAAGSLLALLILWTLFGNVLVCAAIVRYRHLRSKVTNIFIVSLAVSDLLVAVLVMPWKAVAEVAGYWPFGAFQNVWVAFDIMCSTASILNLCVISVDRYWAISSPFRYERKMTQRLALVMIGVAWALSVLISFIPVQLNWHRGGDAATAAAAGDIEDGFDTGWEAAGAFTTWAEDMSTTWVALAAMTPSEGTSGSNNTVPGPSESCDSSLNRTYAISSSLISFYIPVAIMIVTYTRIYRIAQVQIRRISSLERAAEHAQSCRCNHVDCHHHTSLKSSIRKETKVLKTLSIIMGVFVCCWLPFFILNCMVPFCESPPSDPRAGLPCVSETTFNIFVWFGWANSSLNPIIYAFNADFRKVFSNLLGCGQFCSSTPVETVNISNELISYHQDTFHKEIVTAYVNMIPNVVDCEENREDPFDRMSQISPDPEVATDSVCELDCEGEISLGKITPFTPNGLH

SEQ ID NO: 51 - protein, 458 aa, Anguilla anguilla
MGSPAKYLSVHETQSVPFFIGEIMWNTSESAEKTDGKKELIVRTVTGCLLSLLILWTLLGNILVCSAVLKFRHLRTKVTNIFIVSLAVSDLFVAVLVMPWKAVAEVAGYWPFGPFCNIWVAFDIMCSTASILNLCIISVDRYWAISSPFRYERKMTQRVAFVMISVTWTLSVLISFIPVQLNWHKASDEEVWINGTSFGEKSENCDSSLNREYAISSSLISFYIPVAIMIVTYTRIYRIAQIQIRRISSLERAAEHAQSCRTNRLECQHHNTLKTSIKRETKVFKTLSVIMGVFVCCWLPFFILNCIVPFCDRPPTDHTAGLPCVSDTTFDVFVWFGWTNSSLNPIIYAFNADFRKAFASLLGCRNFCSRTPVETVNISNELVSYNQDTLFHKEIVTAYVNMIPNVVDCIDDNEDTFDRISQFSHNNEIATDSVCDLDDCEADICLDRLAPFTPNGLH

SEQ ID NO: 52 - protein, 879 aa, Rattus norvegicus
MKMLTRLQILMLALFSKGFLLSLGDHNFMRREIKIEGDLVLGGLFPINEKGTGTEECGRINEDRGIQRLEAMLFAIDEINKDNYLLPGVKLGVHILDTCSRDTYALEQSLEFVRASLTKVDEAEYMCPDGSYAIQENIPLLIAGVIGGSYSSVSIQVANLLRLFQIPQISYASTSAKLSDKSRYDYFARTVPPDFYQAKAMAEILRFFNWTYVSTVASEGDYGETGIEAFEQEARLRNICIATAEKVGRSNIRKSYDSVIRELLQKPNARVVVLFMRSDDSRELIAAANRVNASFTWVASDGWGAQESIVKGSEHVAYGAITLELASHPVRQFDRYFQSLNPYNNHRNPWFRDFWEQKFQCSLQNKRNHRQVCDKHLAIDSSNYEQESKIMFVVNAVYAMAHALHKMQRTLCPNTTKLCDAMKILDGKKLYKEYLLKINFTAPFNPNKGADSIVKFDTFGDGMGRYNVFNLQQTGGKYSYLKVGHWAETLSLDVDSIHWSRNSVPTSQCSDPCAPNEMKNMQPGDVCCWICIPCEPYEYLVDEFTCMDCGPGQWPTADLSGCYNLPEDYIKWEDAWAIGPVTIACLGFLCTCIVITVFIKHNNTPLVKASGRELCYILLFGVSLSYCMTFFFIAKPSPVICALRRLGLGTSFAICYSALLTKTNCIARIFDGVKNGAQRPKFISPSSQVFICLGLILVQIVMVSVWLILETPGTRRYTLPEKRETVILKCNVKDSSMLISLTYDVVLVILCTVYAFKTRKCPENFNEAKFIGFTMYTTCIIWLAFLPIFYVTSSDYRVQTTTMCISVSLSGFVVLGCLFAPKVHIVLFQPQKNVVTHRLHLNRFSVSGTATTYSQSSASTYVPTVCNGREVLDSTTSSL

SEQ ID NO: 53 - protein, 879 aa, Mus musculus
MKMLTRLQVLMLALFSKGFLVSLGDHNFMRREIKIEGDLVLGGLFPINEKGTGTEECGRINEDRGIQRLEAMLFAIDEINKDNYLLPGVKLGVHILDTCSRDTYALEQSLEFVRASLTKVDEAEYMCPDGSYAIQENIPLLIAGVIGGSYSSVSIQVANLLRLFQIPQISYASTSAKLSDKSRYDYFARTVPPDFYQAKAMAEILRYFNWTYVSTVASEGDYGETGIEAFEQEARLRNICIATAEKVGRSNIRKSYDSVIRELLQKPNARVVVLFMRSDDSRELIAAASRVNASFTWVASDGWGAQESIVKGSEHVAYGAITLELASHPVRQFDRYFQSLNPYNNHRNPWFRDFWEQKFQCSLQNKRNHRQICDKHLAIDSSNYEQESKIMFVVNAVYAMAHALHKMQRTLCPNTTKLCDAMKILDGKKLYKDYLLKINFTAPFNPNKGADSIVKFDTYGDGMGRYNVFNFQHIGGKYSYLKVGHWAETLYLDVDSIHWSRNSVPTSQCSDPCAPNEMKNMQPGDVCCWICIPCEPYEYLVDEFTCMDCGPGQWPTADLSGCYNLPEDYIRWEDAWAIGPVTIACLGFMCTCIVITVFIKHNNTPLVKASGRELCYILLFGVSLSYCMTFFFIAKPSPVICALRRLGLGTSFAICYSALLTKTNCIARIFDGVKNGAQRPKFISPSSQVFICLGLILVQIVMVSVWLILETPGTRRYTLPEKRETVILKCNVKDSSMLISLTYDVVLVILCTVYAFKTRKCPENFNEAKFIGFTMYTTCIIWLAFLPIFYVTSSDYRVQTTTMCISVSLSGFVVLGCLFAPKVHIVLFQPQKNVVTHRLHLNRFSVSGTATTYSQSSASTYVPTVCNGREVLDSTTSSL

SEQ ID NO: 54 - protein, 879 aa, Mus musculus
MKMLTRLQVLMLALFSKGFLVSLGDHNFMRREIKIEGDLVLGGLFPINEKGTGTEECRGINEDRGIQRLEAMLFAIDEINKDNYLLPGVKLGVHILDTCSRDTYALEQSLEFVRASLTKVDEAEYMCPDGSYAIQENIPLLIAGVIGGSYSSVSIQVANLLRLFQIPQISYASTSAKLSDKSRYDYFARTVPPDFYQAKAMAEILRYFNWTYVSTVASEGDYGETGIEAFEQEARLRNICIATAEKVGRSNIRKSYDSVIRELLQKPNARVVVLFMRSDDSRELIAAASRVNASFTWVASDGWGAQESIVKGSEHVAYGAITLELASHPVRQFDRYFQSLNPYNNHRNPWFRDFWEQKFQCSLQNKRNHRQICDKHLAIDSSNYEQESKIMFVVNAVYAMAHALHKMQRTLCPNTTKLCDAMKILDGKKLYKDYLLKINFTAPFNPNKGADSIVKFDTYGDGMGRYNVFNFQHIGGKYSYLKVGHWAETLYLDVDSIHWSRNSVPTSQCSDPCAPNEMKNMQPGDVCCWICIPCEPYEYLVDEFTCMDCGPGQWPTADLSGCYNLPEDYIRWEDAWAIGPVTIACLGFMCTCIVITVFIKHNNTPLVKASGRELCYILLFGVSLSYCMTFFFIAKPSPVICALRRLGLGTSFAICYSALLTKTNCIARIFDGVKNGAQRPKFISPSSQVFICLGLILVQIVMVSVWLILETPGTRRYTLPEKRETVILKCNVKDSSMLISLTYDVVLVILCTVYAFKTRKCPENFNEAKFIGFTMYTTCIIWLAFLPIFYVTSSDYRVQTTTMCISVSLSGFVVLGCLFAPKVHIVLFQPQKNVVTHRLHLNRFSVSGTATTYSQSSASTYVPTVCNGREVLDSTTSSL

SEQ ID NO: 55 - protein, 442 aa, Homo sapiens [sequence omitted]

SEQ ID NO: 56 - protein, 442 aa, Homo sapiens
MGLAMEHGGSYARAGGSSRGCWYYLRYFFLFVSLIQFLIILGLVLFMVYGNVHVSTESNLQATERRAEGLYSQLLGLTASQSNLTKELNFTTRAKDAIMQMWLNARRDLDRINASFRQCQGDRVIYTNNQRYMAAIILSEKQCRDQFKDMNKSCDALLFMLNQKVKTLEVEIAKEKTICTKDKESVLLNKRVAEEQLVECVKTRELQHQERQLAKEQLQKVQALCLPLDKDKFEMDLRNLWRDSIIPRSLDNLGYNLYHPLGSELASIRRACDHMPSLMSSKVEELARSLRADIERVARENSDLQRQKLEAQQGLRASQEAKQKVEKEAQAREAKLQAECSRQTQLALEEKAVLRKERDNLAKELEEKKREAEQLRMELAIRNSALDTCIKTKSQPMMPVSRPMGPVPNPQPIDPASLEEFKRKILESQRPPAGIPVAPSSG

SEQ ID NO: 57 - protein, 438 aa, Rattus norvegicus
MGLSMDRSPYSRTGDRDRGCWYYLRYFFLFVSLIQFLIILGLVLFMIYGNVHATTESSLRATEIRADNLYSQVVGLSAAQANLSKQLNISTLVKDTVMQQLLTTRREVERINASFRQCQGDLITYINYNRFIAAIILSEKQCQEQLKEGNKTCEALLFKLGEKVKTLEMEVVKEKAVCSKDKDSLLAGKRQAEMQQEACGKAREQQKQDQQVTEEQLRKVQSLCLPLDQEKFQADVLNVWRDSLVYRSLDNIGYHYSLMPEFSSLRRTCESLPGIMTTKVEELARGLRAGIERVTRENGELRRQKLELERAIQGEREARTRAGTEAQARETQLRTECARQTQLALEEKAALRTQRDDLERQLEARKRELEQLRTEVDVRISALDTCVKAKSLPAIQPRLPGPPPNPPPIDPASLEEFKKRILESQRPPLVNPAVPPSG

SEQ ID NO: 58 - protein, 438 aa, Mus musculus
MGLSMDRSPYARTGDQQRGCWYYLRYFFLFVSLIQFLIILGLVLFMIYGNVHATTESSLRATEIRADSLYSQVVGLSASQANLSKQLNISLLVKETVMQQLLTTRREMERINASFRQCQGDLITYINYNRFIAAIILSEKQCQEQLKEVNKTCEALLFKLGEKVKTLEMEVAKEKAVCSKDKESLLAGKRQTEEQLEACGKARERQQQEQQVTEENLRKVQSLCIPLDQEKFQADVLSAWRDSLIYRTLETLPYHYQLMPEYASLRRTCESLPGIMTTKIEELARGLRAGIERVTRENAELRRQKLELERAAQAAQEARARAGTEAQARETQLRAECARQTQLALEEKAALRAQRDNLERELEARKRELEQLRTEVDVRISALDTCVKAKSLPAVPPRVSGPPPNPPPIDPASLEEFKKRILESQRLPVVNPAAQPSG

SEQ ID NO: 59 - protein, 438 aa, Mus musculus
MGLSMDRSPYARTGDQQRGCWYYLRYFFLFVSLIQFLIILGLVLFMIYGNVHATTESSLRATEIRADSLYSQVVGLSASQANLSKQLNISLLVKETVMQQLLTTRREMERINASFRQCQGDLITYINYNRFIAAIILSEKQCQEQLKEVNKTCEALLFKLGEKVKTLEMEVAKEKAVCSKDKESLLAGKRQAEEQLEACGKARERQQQEQQVTEENLRKVQSLCIPLDQEKFQADVLSAWRDSLIYRTLETLPYHYQLMPEYASLRRTCESLPGIMPPKIEEMARGVRAGIERVTRENAELRRQKLELERAAQRAQEARARAGTEAQARETQLRAECARQTQLALEEKAALRAQRDNLERELEARKRELEQLRTEVDVRISALDTCVKAKSLPAVPPRVSGPPPNPPPIDPASLEEFKKRILESQRLPVVNPAAQPSG

SEQ ID NO: 60 - protein, 1788 aa, Rattus norvegicus
MLRPMQPTSRTLQRPPRGALEAGGRRNCQDQVAHPNWNTQSVQTPRVRRTLGAPVPPSRKVKAWAPGTDQWPGVSPHCKRSEAEAKPSGSQTVNLTGRANDPCDLDSRVQATSVKVTVAGFQPGGAVEKLCQESLGKLTTGDACVSTSCELASALSHLDASHLTENLPKAASELGQQPMTSSDLISSPGKKGAAHPDPSKTSVDTGQVSRPENPSQPASPRVTKCKARSPVRLPHEGSPSPGEKAAAPPDYSKTRSASETSTPHNTRRVAALRGAGPGAEGMTPAGAVLPGDPLTSQEQRQGAPGNHSKALEMTGIHAPESSQEPSLLEGADSVSSRAPQASLSMLPSTDNTKEACGHVSGHCCPGGSRESPVTDIDSFIKELDASAARSPSSQTGDSGSQEGSAQGHPPAGAGGGSSCRAEPVPGGQTSSPRRAWAAGAPAYPQWASQPSVLDSINPDKHFTVNKNFLSNYSRNFSSFHEDSTSLSGLGDSTEPSLSSMYGDAEDSSSDPESLTEAPRASARDGWSPPRSRVSLHKEDPSESEEEQIEICSTRGCPNPPSSPAHLPTQAAICPASAKVLSLKYSTPRESVASPREKAACLPGSYTSGPDSSQPSSLLEMSSQEHETHADISTSQNHRPSCAEETTEVTSASSAMENSPLSKVARHFHSPPIILSSPNMVNGLEHDLLDDETLNQYETSINAAASLSSFSVDVPKNGESVLENLHISESQDLDDLLQKPKMIARRPIMAWFKEINKHNQGTHLRSKTEKEQPLMPARSPDSKIQMVSSSQKKGVTVPHSPPQPKTNLENKDLSKKSPAEMLLTNGQKAKCGPKLKRLSLKGKAKVNSEAPAANAVKAGGTDHRKPLISPQTSHKTLSKAVSQRLHVADHEDPDRNTTAAPRSPQCVLESKPPLATSGPLKPSVSDTSIRTFVSPLTSPKPVPEQGMWSRFHMAVLSEPDRGCPTTPKSPKCRAEGRAPRADSGPVSPAASRNGMSVAGNRQSEPRLASHVAADTAQPRPTGEKGGNIMASDRLERTNQLKIVEISAEAVSETVCGNKPAESDRRGGCLAQGNCQEKSEIRLYRQVAESSTSHPSSLPSHASQAEQEMSRSFSMAKLASSSSSLQTAIRKAEYSQGKSSLMSDSRGVPRNSIPGGPSGEDHLYFTPRPATRTYSMPAQFSSHFGREGHPPHSLGRSRDSQVPVTSSVVPEAKASRGGLPSLANGQGIYSVKPLLDTSRNLPATDEGDIISVQETSCLVTDKIKVTRRHYCYEQNWPHESTSFFSVKQRIKSFENLANADRPVAKSGASPFLSVSSKPPIGRRSSGSIVSGSLGHPGDAAARLLRRSLSSCSENQSEAGTLLPQMAKSPSIMTLTISRQNPPETSSKGSDSELKKSLGPLGIPTPTMTLASPVKRNKSSVRHTQPSPVSRSKLQELRALSMPDLDKLCSEDYSAGPSAVLFKTELEITPRRSPGPPAGGVSCPEKGGNRACPGGSGPKTSAAETPSSASDTGEAAQDLPFRRSWSVKLDQLLVSAGDQQRLQSVLSSVGSKSTILTLIQEAKAQSENEEDVCFIVLNRKEGSGLGFSVAGGTDVEPKSITVHRVFSQGAASQEGTMNRGDFLLSVNGASLAGLAHGNVLKVLHQAQLHKDALVVIKKGMDQPRPSARQEPPTANGKGLLSRKTIPLEPGIGRSVAVHDALCVEVLKTSAGLGLSLDGGKSSVTGDGPLVIKRVYKGGAAEQAGIIEAGDEILAINGKPLVGLMHFDAWNIMKSVPEGPVQLLIRKHRNSS

SEQ ID NO: 61 - protein, 1608 aa, Homo sapiens
SSDLISSPGKKGAAHPDPSKTSVDTGKVSRPENPSQPASPRVAKCKARSPVRLPHEGSPSPGEKAAAPPDYSKTRSASETSTPHNTRRVAALRGAGPGAEGMTPAGAVLPGDPLTSQEQRQGAPGNHSKALEMTGIHAPESSQEPSLLEGADSVSSRAPQASLSMLPSTDNTKEACGHVSGHCCPGGSRESPVTDIDSFIKELDASAARSPSSQTGDSGSQEGSAQGHPPAGAGGGSSCRAEPVPGGQTSSPRRAWAAGAPAYPQWASQPSVLDSINPDKHFTVNKNFLSNYSRNFSSFHEDSTSLSGLGDSTEPSLSSMYGDAEDSSSDPESLTEAPRASARDGWSPPRSRVSLHKEDPSESEEEQIEICSTRGCPNPPSSPAHLPTQAAICPASAKVLSLKYSTPRESVASPREKVACLPGSYTSGPDSSQPSSLLEMSSQEHETHADISTSQNHRPSCAEETTEVTSASSAMENSPLSKVARHFHSPPIILSSPNMVNGLEHDLLDDETLNQYETSINAAASLSSFSVDVPKNGESVLENLHISESQDLDDLLQKPKMIARRPIMAWFKEINKHNQGTHLRSKTEKEQPLMPARSPDSKIQMVSSSQKKGVTVPHSPPQPKTNLENKDLSKKSPAEMLLTNGQKAKCGPKLKRLSLKGKAKVNSEAPAANAVKAGGTDHRKPLISPQTSHKTLSKAVSQRLHVADHEDPDRNTTAAPRSPQCVLESKPPLATSGPLKPSVSDTSIRTFVSPLTSPKPVPEQGMWSRFHMAVLSEPDRGCPTTPKSPKCRAEGRAPRADSGPVSPAASRNGMSVAGNRQSEPRLASHVAADTAQPRPTGEKGGNIMASDRLERTNQLKIVEISAEAVSETVCGNKPAESDRRGGCLAQGNCQEKSEIRLYRQVAESSTSHPSSLPSHASQAEQEMSRSFSMAKLASSSSSLQTAIRKAEYSQGKSSLMSDSRGVPRNSIPGGPSGEDHLYFTPRPATRTYSMPAQFSSHFGREGHPPHSLGRSRDSQVPVTSSVVPEAKASRGGLPSLANGQGIYSVKPLLDTSRNLPATDEGDIISVQETSCLVTDKIKVTRRHYCYEQNWPHESTSFFSVKQRIKSFENLANADRPVAKSGASPFLSVSSKPPIGRRSSGSIVSGSLGHPGDAAARLLRRSLSSCSENQSEAGTLLPQMAKSPSIMTLTISRQNPPETSSKGSDSELKKSLGPLGIPTPTMTLASPVKRNKSSVRHTQPSPVSRSKLQELRALSMPDLDKLCSEDYSAGPSAVLFKTELEITPRRSPGPPAGGVSCPEKGGNRACPGGSGPKTSAAETPSSASDTGEAAQDLPFRRSWSVNLDQLLVSAGDQQRLQSVLSSVGSKSTILTLIQEAKAQSENEEDVCFIVLNRKEGSGLGFSVAGGTDVEPKSITVHRVFSQGAASQEGTMNRGDFLLSVNGASLAGLAHGNVLKVLHQAQLHKDALVVIKKGMDQPRPSARQEPPTANGKGLLSRKTIPLEPGIGRSVAVHDALCVEVLKTSAGLGLSLDGGKSSVTGDGPLVIKRVYKGGAAEQAGIIEAGDEILAINGKPLVGLMHFDAWNIMKSVPEGPVQLLIRKHRNSS

SEQ ID NO: 62 - protein, 2766 aa, Rattus norvegicus
MPITQDNALLHLPLLYEWLQNSLREGGDSPEQRLCQAAIQKLQEYIQLNLAVDESTVPPDHSPPEMEICTVYLTKQLGDTETVGLSFGNIPVFGDYGEKRRGGKKRKTHQGPVLDVGCIWVTELRKNSPAGKSGKVRLRDEILSLNGQLMVGVDVTGASYLAEQCWNGGFIYLIMLRRFKQKAHVTYNGNSGNSSEPGETPTLELGDQTSKKGKRTRKFGVISRPSISKTPEDSKSSSGCDTADDPNSELENGADPELGNGHAFELENGPHSLKDVAGPHLERSEADSEVELRVPKTEAPLSDSNDKRRFSKTGKTDFQSSDCLAREEVGRIWKMELLKESDGLGIQVSGGRGSKRSPHAIVVTQVKEGGAAHRDGRLSLGDELLVINGHLLVGLSHEEAVAILRSATGMVQLVVASKMPGSEESQDVGSSEESKGNLESPKQGNCKTKLKSRLSGGVHRLESVEEYNELMVRNGDPRIRMLEVSRDGRKHSLPQLLDSTGTSQEYHIVKKSTRSLSTTHVESPWRLIRPSVISIIGLYKEKGKGLGFSIAGGRDCIRGQMGIFVKTIFPNGSAAEDGRLKEGDEILDVNGIPIKGLTFQEAIHTFKQIRSGLFVLTVRTKLLSPSLTPCSTPTHMSRSSSPSFNTNSGGTPAGGGQEEGGSSSLGRKAPGPKDRIVMEVTLNKEPRVGLGIGACCLALENSPPGIYIHSLAPGSVAKMESNLSRGDQILEVNSVNVRHAALSKVHAILSKCPPGPVRLVIGRHPNPKVSEQEMDEVIARSTYQESREANSSPGLGTPLKSPSLAKKDSLLSESELSQYFVHDGQGSLSDFVVAGSEDEDHPGSGYETSEDGSLLPVPSAHKARANSLVTLGSQRTSGLLHKQVTVARQASLPGSPQVLRNPLLRQRRVRCYDSNGGSDDEDFDGEGDCISLPGVLPGPGKPLVEDDTRPALTTSSKSIDVNKQEERLQKPLVSKACSVPLLGSSLDSEHSILNGAGGTPPKVASLPGSGETPKNGPRGSGRKEMSGSRSSPKLEYRVPTDTQSPRSPENHTSPPQKSENLVSRHKPVARISPHYKRSDAEEAPGGTANGPCAQDLKVQASPVKDPVTSRQPGGTAEKELRGNPTPGDSSVPTNCGPASTPCHPNIGLPTENPQGAAPECGPHPGTGWDGSSEHLCSPGKSREVHPDSSETPTVAEQVHQPESLSQPVSPRTSEPESQGISKMKPPSQRCVSPREKASTPPDSSRAWAAPGDSSPSTRRIAVPMSTGAAPATAIPQASLVSQERSRGLSGPSKGLGTKELCIPKSLKDGALLEDTAPASGKMSHASSPSGPVATERTLSGSPENPVTDIDNFIEEASEARLSQSPQKADCRAHGDTFESQPPGGAGSSSSHHAQMVRSDQTSSPRKTGGTGSPPPQQWALQPSVLDSIHPDKHLAVNKTFLNNYSRNFSNFHEDSISLSGPGGSSEPSPSSMYGNAEDSSSDPESLAEDPGAAARNNWSPPLSPESSPKEGSSESEDERIEICSTDGCPGTPVTAPPPTQVALCPVLPVQQRAVCKPVGDICERACFVPGASRTSIPDSSQPFSFLDVSSEEPETWASINASQNHMPVCTEGIMDVTSTSSNMGDSQSSQMTRHCRNAPFVLGNPDMVNDLGRDLLDEGAPKEGAAAASVMRSVFALGAEGPKNGEAVLADLHIAERGNLEDLLQKPKTISRRPILTWFKEINKDSQGSHLRSTSEKEQSSMLALGPGSKANMVNTGHRKGVTVPKSPPSRQKSQENKDLPPKSPVETLGNCQKPKCSPKLKRLNSKGKASPEVPVAISTKGSRNDHRKTLPSPQASHKMFSKAVSHRLHIADQEEPKNTAGDTPKPPQCVPESKPPQAALGSLRTSASDTSIRTFTSPLTSPKLLPEQGANSRFHMAVYLESDTSCPTTSRSPRSGPEGKAPHANSGSASPPASRASLALAGIRQSKQFTPGRADLLVSEATQPQGICEKGAEKKVSDPPQRTNQLKIVEISSERVPKNACGDRPPESDRKGGFLTQNNCQEKSAIRLRQSEESSPEHTPFPPSQASQVEREIRWSFSMAKPATSSSSSLQLPAKLPESFQGKSSQMPASVGVPKNGVPIGLAGEESPYFTPRPATRTYSMPAQFSSHFGREGPSPHSPSHSPQDPQVPAMGGKLSEKTAKGVTNGQGVYSVKPLLETSKNLSPVDGRDVSADPETSCLIPDKVKVTRRQYCCEQSWPHESTSFFSVKQRIKSFENLANSDRPTAKCATSPFLSVSSKPPINRRSSGSIPSGSPSDMTSRSLRRSLSSCSESQSEASSLLPQMTKSPSSMTLTVSRQNPPDTSNKGPSPDPKKSLVPVGIPTSTVSPASPSKRNKSSVRHAQPSPVSRSKLQERRTLSMPDLDKLCNGEDDSASPGAVLFKTQLEITPRRSKGSQATSPAGSPARGHADFNGSTFLSCPMNGGTRAYTKGNSPPASEPAIATGSREEGESVWATPSGKSWSVSLDRLLASVGNQQRLQGILSLVGSKSPILTLIQEAKAQSETKEDICFIVLNKKEGSGLGFSVAGGADVEPKSVMVHRVFSQGVASQEGTVSRGDFLLSVNGTSLAGLAHSEVTKVLHQAELHKHALMIIKKGNDQPGPSFKQEPPSANGKGPFPRRTLPLEPGAGRNGAAHDALCVEVLKTSAGLGLSLDGGKSSVSGEGPLVIKRVYKGGAAERAGTIEAGDEILAINGKPLVGLVHFDAWNIMKSVPEGPVQLVIRKHRDS

SEQ ID NO: 63 - protein, 2641 aa, Homo sapiens [sequence omitted]

SEQ ID NO: 64 - protein, 364 aa, Mus musculus
MLRRFKQKAHLTYNGNSGNSSEPGETPTLELGDQTSKKGKRTRKFGVISRPAIIKAPEDSKSNSGCDTADDPSSELENGTDSELGNGHAFELENGPNSLKDVAGPHLERSEADREAELRVPKTEAPLSDSNDKRRFSKTGKTNFQSSDSLAREEVGRIWEMELLKESDGLGIQVSGGRGSKRSPHAIVVTQVKEGGAAHRDGRLSLGDELLVINGHLLVGLSHEEAVAILRSATGMVQLVVASKMLGSEESQDVGSSEESKGNNLESPKQGNSKMKLKSRLSGGVHRLESVEEYNELMVRNGDPRIRMLEVSRDGRKHSLPQLLDSTGTSQEYHIVKKSTRSLSTTHVESPWRLIRPSVISIIG

SEQ ID NO: 65 - protein, 51 aa, Artificial Sequence
  Description of Artificial Sequence LRRCT Consensus Sequence
NPFICDCELRWLLRWLQANRHLQDPVDLRCASPESLRGPLLLLLPSSFKCP

SEQ ID NO: 66 - protein, 51 aa, Artificial Sequence
  Description of Artificial Sequence LRRCT Consensus Sequence
NPFICDCELRWLLRWLREPRRLEDPEDLRCASPESLRGPLLELLPSDFSCP

SEQ ID NO: 67 - protein, 124 aa, Artificial Sequence
  Description of Artificial Sequence CLECT Consensus Sequence
CPSGWVSYPGGKCYKFSTEKKTWADAQAFCQSLGAHLASIHSEEENDFLLSLLKNSNSDYYWIGLSRPDSNGSWQWSDGSGPVDYSNWAPGEPGGSGNCVVLSTSGGGKWNDVSCTSKLPFICE

SEQ ID NO: 68 - protein, 114 aa, Artificial Sequence
  Description of Artificial Sequence CUB Consensus Sequence
CGGTLTASSGTITSPNYPNSYPNNLNCVWTISAPPGYRIELKFTDFDLESSDNCTYDYVEIYDGPSTSSPLLGRFCGSELPPPIISSSSNSMTVTFVSDSSVQKRGFSARYSAV

SEQ ID NO: 69 - protein, 110 aa, Artificial Sequence
  Description of Artificial Sequence CUB Consensus Sequence
CGGVLTESSGSISSPNYPNDYPPNKECVWTIRAPPGYRVELTFQDFDLEDHTGCRYDYVEIRDGDGSSSPLLGKFCGSGPPEDIVSSSNRMTIKFVSDASVSKRGFKATY

SEQ ID NO: 70 - protein, 107 aa, Artificial Sequence
  Description of Artificial Sequence Lectin C-type domain Consensus Sequence
ESKTWAEAQAACQKLGGGLVSIQSAEEQDFLTSLTKASNSYAWIGLTDINTEGTWVWTDGSPVNYTNWAPGEPNNRGNKEDCVEIYTDGNKWNDEPCGSKLPYVCEF

SEQ ID NO: 71 - protein, 304 aa, Artificial Sequence
  Description of Artificial Sequence CNH domain Consensus Sequence
YTAKCNHPITCDAKNLLVGTEEGLYVLNRSDQGGTLEKIISRRSVTQIWVLEENNVLLMISGKKPYLYAHPLSGLREKDALGSARLVIRKNVWVKIEDVKGCHLFAVVNGKRILFLCAALPSSVQLLQWYNPLKKFKLFKSKFLKKLIVPVPLFVLLTSSSFELPKICIGVDKNGFDVVQFHQTSLVSKEDLSLPNLNEETSKKPIPVIQVPQSDDELLLCFDEFGVFVNLQGMRRSRKPILTWEFMPEYFAYHEPYLLAFHSNGIEIRSIETGELLQELADREARKIRVLGSSDRKILVSSSP

SEQ ID NO: 72 - protein, 256 aa, Artificial Sequence
  Description of Artificial Sequence Serine/Threoniune protein kinase Consensus Sequence
YELLEVLGKGAFGKVYLARDKKTGKLVAIKVIKKEKLKKKKRERILREIKILKKLDHPNIVKLYDVFEDDDKLYLVMEYCEGGDLFDLLKKRGRLSEDEARFYARQILSALEYLHSQGIIHRDLKPENILLDSDGHVKLADFGLAKQLDSGGTLLTTFVGTPEYMAPEVLLGKGYGKAVDIWSLGVILYELLTGKPPFPGDDQLLALFKKIGKPPPPFPPPEWKISPEAKDLIKKLLVKDPEKRLTAEEALEHPFF

SEQ ID NO: 73 - protein, 301 aa, Artificial Sequence
  Description of Artificial Sequence CNH domain Consensus Sequence
NTAKWNHPITCDAKILLVGTEEGLYVLNISDQHGTLEKLIGRRSVTQIWVLEENNVLLMISGKKPQLYSHPLSALTEKDALGSARLVIRKNVLTKIPDVKGCHLCAVVNGKRILFLCHALQSSVVLLQWYNPLKKFKLFKSKFLFPLISPVPVFVELVSSSFELPGICIGSDKNGGDVVQFHQSLVSKEDLSLPFLSEETSSKPISVVQVPADELLLCYDEFGVFVNLYGMRRSRNPILHWEFMPESFAYHSPYLLAFHDNGIEIRSIKTGELLQELADRKTRKIRLLGSSDRKILLSSSP

SEQ ID NO: 74 - protein, 256 aa, Artificial Sequence
  Description of Artificial Sequence Protein kinase domain Consensus Sequence
YELGEKLGSGAFGKVYKGKHKDTGEIVAIKILKKRSLSEKKKRFLREIQILRRLSHPNIVRLLGVFEEDDHLYLVMEYMEGGDLFDYLRRNGLLLSEKEAKKIALQILRGLEYLHSRGIVHRDLKPENILLDENGTVKIADFGLARKLESSSYEKLTTFVGTPEYMAPEVLEGRGYSSKVDVWSLGVILYELLTGKLPFPGIDPLEELFRIKERPRLRLPLPPNCSEELKDLIKKCLNKDPEKRPTAKEILNHPWF

SEQ ID NO: 75 - protein, 258 aa, Artificial Sequence
  Description of Artificial Sequence Tyrosine Kinase Consensus Sequence
LTLGKKLGEGAFGEVYKGTLKGKGGVEVEVAVKTLKEDASEQQIEEFLREARLMRKLDHPNIVKLLGVCTEEEPLMIVMEYMEGGDLLDYLRKNRPKELSLSDLLSFALQIARGMEYLESKNFVHRDLAARNCLVGENKTVKIADFGLARDLYDDDYYRKKKSPRLPIRWMAPESLKDGKFTSKSDVWSFGVLLWEIFTLGESPYPGMSNEEVLEYLKKGYRLPQPPNCPDEIYDLMLQCWAEDPEDRPTFSELVERL

SEQ ID NO: 76 - protein, 430 aa, Artificial Sequence
  Description of Artificial Sequence Sema domain Consensus Sequence
LQNLLLDEDNGTLYVGARNRLYVLSLNLISEAEVKTGPVLSSPDCEECVSKGKDPPTDCVNFIRLLLDYNADHLLVCGTNAFQPVCRLINLGNLDRLEVGRESGRGRCPFDPQHNSTAVLVDGELYVGTVADFSGSDPAIYRSLSVRRLKGTSGPSLRTVLYDSRWLNEPNFVYAFESGDFVYFFFRETAVEDENCGKAVVSRVARVCKNDVGGPRSLSKKWTSFLKARLECSVPGEFPFYFNELQAAFLLPAGSESDDVLYGVFSTSSNPIPGSAVCAFSLSDINAVFNEPFKECETGNSQWLPYPRGLVPFPRPGTCPNTPLSSKDLPDDVLNFIKTHPLMDEVVQPLTGRPLFVKTDSNYLLTSIAVDRVRTDGGNYTVLFLGTSDGRILKVVLSRSSSSSESVVLEEISVFDPGSPVSDLVLSPKK

SEQ ID NO: 77 - protein, 433 aa, Artificial Sequence
  Description of Artificial Sequence Sema domain Consensus Sequence
FVTLLLDEDRGRLYVGARNRVYVLNLEDLSEVLNLKTGWPGSCETCEECNMKGKSPLTECTNFIRVLQAYNDTHLYVCGTNAFQPVCTLINLGDLFSLDVDNEEDGCGDCPYDPLGNTTSVLVQGGELYSGTVIDFSGRDPSIRRLLGSHDGLRTEFHDSKWLNLPNFVDSYPIHYVHSFSDDKVYFFFRETAVEDSNCKTIHSRVARVCKNDPGGRSYLELNKWTTFLKARLNCSIPGEGTPFYFNELQAAFVLPTGADTDPVLYGVFTTSSNSSAGSAVCAFSMSDINQVFEGPFKHQSPNSKWLPYRGKVPQPRPGQCPNASGLNLPDDTLNFIRCHPLMDEVVPPLHNVPLFVGQSGNYRLTSIAVDRVRAGDGQIYTVLFLGTDDGRVLKQVVLSRSSSASYLVVVLEESLVFPDGEPVQRMVISSKN

SEQ ID NO: 78 - protein, 85 aa, Artificial Sequence
  Description of Artificial Sequence TIG domain Consensus Sequence
PVITSISPSSGPLSGGTEITITGSNLGSGEDIKVTFGGTECDVVSQEASQIVCKTPPYANGGPQPVTVSLDGGGLSSSPVTFTYV

SEQ ID NO: 79 - protein, 85 aa, Artificial Sequence; identical to SEQ ID NO: 78
  Description of Artificial Sequence TIG Consensus Sequence

SEQ ID NO: 80 - protein, 85 aa, Artificial Sequence; identical to SEQ ID NO: 78
  Description of Artificial Sequence TIG Consensus Sequence

SEQ ID NO: 81 - protein, 47 aa, Artificial Sequence
  Description of Artificial Sequence PSI domain Consensus Sequence
RCSAYTSCSECLLARDPYCAWCSSQGRCTSGERCDSLRQNWSSGQCP

SEQ ID NO: 82 - protein, 93 aa, Artificial Sequence
  Description of Artificial Sequence IPT Consensus Sequence
DPVITRISPNSGPLSGGTRITLCGKNLDSISVVFVEVGVGEVPCTFLPSDVSQTAIVCKTPPYHNIPGSVPVRVEVGLRNGGVPGEPSPFTYV

SEQ ID NO: 83 - protein, 254 aa, Artificial Sequence
  Description of Artificial Sequence 7 transmembrane receptor Consensus Sequence
GNLLVILVILRTKKLRTPTNIFLLNLAVADLLFLLTLPPWALYYLVGGDWVFGDALCKLVGALFVVNGYASILLLTAISIDRYLAIVHPLRYRRIRTPRRAKVLILLVWVLALLLSLPPLLFSWLRTVEEGNTTVCLIDFPEESVKRSYVLLSTLVGFVLPLLVILVCYTRILRTLRKRARSQRSLKRRSSSERKAAKMLLVVVVVFVLCWLPYHIVLLLDSLCLLSIWRVLPTALLITLWLAYVNSCLNPIIY

SEQ ID NO: 84 - protein, 402 aa, Artificial Sequence
  Description of Artificial Sequence ANF receptor Consensus Sequence
GLINYAVRGITRLEAMLGAFDRINADPALLPGLALGLAILDINSLRNVALEQSFTFVYGLLIKCDCSSVRCAGGDLALTHGVAGVIGPSCSSSAIQVANLASLLNIPMISYGSTAPELSDKTRYPTFSRTIPSDAFQGLAMVDIFKHFNWNYVSVVYSDGTYGEEGCEAFIEALRARGGCIALSVKIGEFDRGDEEDFDKLLRELKRRARVVVMCGHGETLRELLEAALRLGLTGEDYVFISDDLFNKSLPAEPGAPGAIELANASMLRFAYYFVLVLTLNNPRNPWFLEFWKENFICALQDFLTLEPYEQEGKAGFVYDAVYLYAHALHNTTLALGGSWVDGEKLVQHLRNLTFEGVTGPVTFDENGDRDGDYVLLDTQNTETGQLKVTGTYDGVGKWTEP

SEQ ID NO: 85 - protein, 256 aa, Artificial Sequence
  Description of Artificial Sequence 7 transmembrane receptor (metabotrobic E family) Consensus Sequence
LGIVLVALAVLGIVLTLFVLVVFVKHRDTPIVKASNRELSYLLLIGLILCYLCSFLFIGKPSETSCILRRILFGLGFTLCYSALLAKTNRVLRIFRAKKPGSGKPKFISPWAQVLIVLILVLIQVIICVIWLVVEPPRPTIDIYSEKEKIILECNKGSMVAFVVVLGYDGLLAVLCTFLAFLTRNLPENFNEAKFIGFSMLTFCIVWVAFIPIYLSTPGKVQVAVEIFSILASSTVLLGCLFVPKCYIILFRPEKN

SEQ ID NO: 86 - protein, 86 aa, Artificial Sequence
  Description of Artificial Sequence PDZ domain Consensus Sequence
EPRLVELEKGGGGLGFSLVGGKDSGDGGVVVSSVVPGSPAAKAGLKPGDVILEVNGTSVEGLTHLEAVDLLKEAGGKVTLTVLRGG

SEQ ID NO: 87 - DNA, 25 bp, Artificial Sequence
  Description of Artificial Sequence NOV1c Primer 1
tcatcacatgacaacatgaagctgt

SEQ ID NO: 88 - DNA, 26 bp, Artificial Sequence
  Description of Artificial Sequence NOV1c Primer 2
gaaagccctcaaactctccatctatg

SEQ ID NO: 89 - DNA, 22 bp, Artificial Sequence
  Description of Artificial Sequence NOV7a Primer 1
ccaatctctgatgccctgcgat

SEQ ID NO: 90 - DNA, 20 bp, Artificial Sequence
  Description of Artificial Sequence NOV7a Primer 2
aggtcagtgccggagcctcc

SEQ ID NO: 91 - DNA, 19 bp, Artificial Sequence
  Description of Artificial Sequence Ag273b Forward
cggcttgacgatgcttcac

SEQ ID NO: 92 - DNA, 32 bp, Artificial Sequence
  Description of Artificial Sequence Ag273b Probe
tgacttttctgggcttaccaatgctatttcaa

SEQ ID NO: 93 - DNA, 27 bp, Artificial Sequence
  Description of Artificial Sequence Ag273b Reverse
gcacctatctcaatatctgcaatattg

SEQ ID NO: 94 - DNA, 20 bp, Artificial Sequence
  Description of Artificial Sequence Ag1094 Forward
atggactggaaaacctggaa

SEQ ID NO: 95 - DNA, 29 bp, Artificial Sequence
  Description of Artificial Sequence Ag1094 Probe
tcctgcaagcagataacaattttatcaca

SEQ ID NO: 96 - DNA, 20 bp, Artificial Sequence
  Description of Artificial Sequence Ag1094 Reverse
tgctaaaggcacttggttca

SEQ ID NO: 97 - DNA, 21 bp, Artificial Sequence
  Description of Artificial Sequence Ag2100 Forward
agatccctggaacagaggatt

SEQ ID NO: 98 - DNA, 26 bp, Artificial Sequence
  Description of Artificial Sequence Ag2100 Probe
tgtctgaagccaataaacttgcagca

SEQ ID NO: 99 - DNA, 21 bp, Artificial Sequence
  Description of Artificial Sequence Ag2100 Reverse
ccttcatgttcctttgggtaa

SEQ ID NO: 100 - DNA, 22 bp, Artificial Sequence
  Description of Artificial Sequence Ag217 Forward
atctgtgctgaggcatgttcct

SEQ ID NO: 101 - DNA, 23 bp, Artificial Sequence
  Description of Artificial Sequence Ag217 Probe
atcctcctccctccccggctctc

SEQ ID NO: 102 - DNA, 19 bp, Artificial Sequence
  Description of Artificial Sequence Ag217 Reverse
ctgcatggctggtgtgatg

SEQ ID NO: 103 - DNA, 22 bp, Artificial Sequence
  Description of Artificial Sequence Ag850 Forward
cctttcttctcttcctcctcaa

SEQ ID NO: 104 - DNA, 23 bp, Artificial Sequence
  Description of Artificial Sequence Ag850 Probe
cacctggcgagtgctcctctctg

SEQ ID NO: 105 - DNA, 19 bp, Artificial Sequence
  Description of Artificial Sequence Ag850 Reverse
ggtggatggcgttgtagag

SEQ ID NO: 106 - DNA, 22 bp, Artificial Sequence
  Description of Artificial Sequence Ag1469 Forward
cgtacgtcttccatgatgagtt

SEQ ID NO: 107 - DNA, 26 bp, Artificial Sequence
  Description of Artificial Sequence Ag1469 Probe
cgtggcctcgatgattaagatccctt

SEQ ID NO: 108 - DNA, 21 bp, Artificial Sequence
  Description of Artificial Sequence Ag1469 Reverse
aagtcagggatgatggtgaag

SEQ ID NO: 109 - DNA, 20 bp, Artificial Sequence
  Description of Artificial Sequence Ag2976 Forward
accccaaatggattccatta

SEQ ID NO: 110 - DNA, 26 bp, Artificial Sequence
  Description of Artificial Sequence Ag2976 Probe
ccctcatggatctgcataaccacaca

SEQ ID NO: 111 - DNA, 20 bp, Artificial Sequence
  Description of Artificial Sequence Ag2976 Reverse
cttgtgtgtgcatgcttgtc

SEQ ID NO: 112 - DNA, 22 bp, Artificial Sequence
  Description of Artificial Sequence Ag760 Forward
caccatgacaacgacacctata

SEQ ID NO: 113 - DNA, 25 bp, Artificial Sequence
  Description of Artificial Sequence Ag760 Probe
atatggcaccaacatcacatgcacg

SEQ ID NO: 114 - DNA, 22 bp, Artificial Sequence
  Description of Artificial Sequence Ag760 Reverse
tgggtagaaagtgtgtgtgaaa

SEQ ID NO: 115 - DNA, 22 bp, Artificial Sequence
  Description of Artificial Sequence Ag1537 Forward
aaggagctggaagagaagaaga

SEQ ID NO: 116 - DNA, 26 bp, Artificial Sequence
  Description of Artificial Sequence Ag1537 Probe
atcagaaactcagccctggacacctg

SEQ ID NO: 117 - DNA, 19 bp, Artificial Sequence
  Description of Artificial Sequence Ag1537 Reverse
gctgcgacttggtcttgat

SEQ ID NO: 118 - DNA, 22 bp, Artificial Sequence
  Description of Artificial Sequence Ag147 Forward
tgaagacagcacctccctatca

SEQ ID NO: 119 - DNA, 21 bp, Artificial Sequence
  Description of Artificial Sequence Ag147 Probe
cggctccgtgctgtcacccag

SEQ ID NO: 120 - DNA, 22 bp, Artificial Sequence
  Description of Artificial Sequence Ag147 Reverse
aagaatcctcagcatcgccata

SEQ ID NO: 121 - DNA, 22 bp, Artificial Sequence
  Description of Artificial Sequence Ag718 Forward
agaaggaatctctgggaaagct

SEQ ID NO: 122 - DNA, 27 bp, Artificial Sequence
  Description of Artificial Sequence Ag718 Probe
ccactggagatgcttgtgtctctacca

SEQ ID NO: 123 - DNA, 22 bp, Artificial Sequence
  Description of Artificial Sequence Ag718 Reverse
gacagagcactggctagttcac

SEQ ID NO: 124 - DNA, 22 bp, Artificial Sequence
  Description of Artificial Sequence Ag3681/Ag4085 Forward
gaatcatccacaagtcatccat

SEQ ID NO: 125 - DNA, 25 bp, Artificial Sequence
  Description of Artificial Sequence Ag3681/Ag4085 Probe
ctcactcccatctcatgcctcccag

SEQ ID NO: 126 - DNA, 20 bp, Artificial Sequence
  Description of Artificial Sequence Ag3681/Ag4085 Reverse
catgctgaatgatcgtgaca

SEQ ID NO: 127 - protein, 1011 aa, Homo sapiens
ISNSSDTVECECSENWKGEACDIPHCTDNCGFPHRGICNSSDVRGCSCFSDWQGPGCSVPVPANQSFWTREEYSNLKLPRASHKAVVNGNIMWVVGGYMFNHSDYNMVLAYDLASREWLPLNRSVNNVVVRYGHSLALYKDKIYMYGGKIDSTGNVTNELRVFHIHNESWVLLTPKAKEQYAVVGHSAHIVTLKNGRVVMLVIFGHCPLYGYISNVQEYDLDKNTWSILHTQGALVQGGYGHSSVYDHRTRALYVHGGYKAFSANKYRLADDLYRYDVDTQMWTILKDSRFFRYLHTAVIVSGTMLVFGGNTHNDTSMSHGAKCFSSDFMAYDIACDRWSVLPRPDLHHDVNRFGHSAVLHNSTMYVFGGFNSLLLSDILVFTSEQCDAHRSEAACLAAGPGIRCVWNTGSSQCISWALATDEQEEKLKSECFSKRTLDHDRCDQHTDCYSCTANTNDCHWCNDHCVPRNHSCSEGQISIFRYENCPKDNPMYYCNKKTSCRSCALDQNCQWEPRNQECIALPENICGIGWHLVGNSCLKITTAKENYDNAKLFCRNHNALLASLTTQKKVEFVLKQLRIMQSSQSMSKLTLTPWVGLRKINVSYWCWEDMSPFTNSLLQWMPSEPSDAGFCGILSEPSTRGLKAATCINPLNGSVCERPANHSAKQCRTPCALRTACGDCTSGSSECMWCSNMKQCVDSNAYVASFPFGQCMEWYTMSTCPPENCSGYCTCSHCLEQPGCGWCTDPSNTGKGKCIEGSYKGPVKMPSQAPTGNFYPQPLLNSSMCLEDSRYNWSFIHCPACQCNGHSKCINQSICEKCENLTTGKHCETCISGFYGDPTNGGKCQPCKCNGHASLCNTNTGKCFCTTKGVKGDECQLCEVENRYQGNPLRGTCYYTLLIDYQFTFSLSQEDDRYYTAINFVATPDEQNRDLDMFINASKNFNLNITWAASFSAGTQAGEEMPVVSKTNIKEYKDSFSNEKFDFRNHPNITFFVYVSNFTWPIKIQVQTEQ